SEQUENCE LISTING (12 sequences)

SEQ ID NO: 1 - protein, 711 aa, synthetic construct
MHHHHHHHHHMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSGLEVLFQGPMENFQKVEKIGEGTYGVVYKARNKLTGEVVALKKIRLDTETEGVPSTAIREISLLKELNHPNIVKLLDVIHTENKLYLVFEFLHQDLKKFMDASALTGIPLPLIKSYLFQLLQGLAFCHSHRVLHRDLKPQNLLINTEGAIKLADFGLARAFGVPVRTYTHEVVTLWYRAPEILLGCKYYSTAVDIWSLGCIFAEMVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRLGGSGGGGSGLNDIFEAQKIEWHE

SEQ ID NO: 2 - protein, 801 aa, synthetic construct
MHHHHHHHHHMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSENLYFQGGSGMPRERRERDAKERDTMKEDGGAEFSARSRKRKANVTVFLQDPDEEMAKIDRTARDQCGSQPWDNNAVCADPCSLIPTPDKEDDDRVYPNSTCKPRIIAPSRGSPLPVLSWANREEVWKIMLNKEKTYLRDQHFLEQHPLLQPKMRAILLDWLMEVCEVYKLHRETFYLAQDFFDRYMATQENVVKTLLQLIGISSLFIAAKLEEIYPPKLHQFAYVTDGACSGDEILTMELMIMKALKWRLSPLTIVSWLNVYMQVAYLNDLHEVLLPQYPQQIFIQIAELLDLCVLDVDCLEFPYGILAASALYHFSSSELMQKVSGYQWCDIENCVKWMVPFAMVIRETGSSKLKHFRGVADEDAHNIQTHRDSLDLLDKARAKKAMLSEQNRASPLPSGLLTPPQSGKKQSSGPEMA

SEQ ID NO: 3 - protein, 709 aa, synthetic construct
MHHHHHHHHHMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSENLYFQGGMEDYTKIEKIGEGTYGVVYKGRHKTTGQVVAMKKIRLESEEEGVPSTAIREISLLKELRHPNIVSLQDVLMQDSRLYLIFEFLSMDLKKYLDSIPPGQYMDSSLVKSYLYQILQGIVFCHSRRVLHRDLKPQNLLIDDKGTIKLADFGLARAFGIPIRVYTHEVVTLWYRSPEVLLGSARYSTPVDIWSIGTIFAELATKKPLFHGDSEIDQLFRIFRALGTPNNEVWPEVESLQDYKNTFPKWKPGSLASHVKNLDENGLDLLSKMLIYDPAKRISGKMALNHPYFNDLDNQIKKMGGSGGGGSGLNDIFEAQKIEWHE

SEQ ID NO: 4 - protein, 822 aa, synthetic construct
MHHHHHHHHHMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSENLYFQGGMALRVTRNSKINAENKAKINMAGAKRVPTAPAATSKPGLRPRTALGDIGNKVSEQLQAKMPMKKEAKPSATGKVIDKKLPKPLEKVPMLVPVPVSEPVPEPEPEPEPEPVKEEKLSPEPILVDTASPSPMETSGCAPAEEDLCQAFSDVILAVNDVDAEDGADPNLCSEYVKDIYAYLRQLEEEQAVRPKYLLGREVTGNMRAILIDWLVQVQMKFRLLQETMYMTVSIIDRFMQNNCVPKKMLQLVGVTAMFIASKYEEMYPPEIGDFAFVTDNTYTKHQIRQMEMKILRALNFGLGRPLPLHFLRRASKIGEVDVEQHTLAKYLMELTMLDYDMVHFPPSQIAAGAFCLALKILDNGEWTPTLQHYLSYTEESLLPVMQHLAKNVVMVNQGLTKHMTVKNKYATSKHAKISTLPQLNSALVQDLAKAVAKV

SEQ ID NO: 5 - protein, 717 aa, synthetic construct
MHHHHHHHHHMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSENLYFQGGSGMATSRYEPVAEIGVGAYGTVYKARDPHSGHFVALKSVRVPNGGGGGGGLPISTVREVALLRRLEAFEHPNVVRLMDVCATSRTDREIKVTLVFEHVDQDLRTYLDKAPPPGLPAETIKDLMRQFLRGLDFLHANCIVHRDLKPENILVTSGGTVKLADFGLARIYSYQMALTPVVVTLWYRAPEVLLQSTYATPVDMWSVGCIFAEMFRRKPLFCGNSEADQLGKIFDLIGLPPEDDWPRDVSLPRGAFPPRGPRPVQSVVPEMEESGAQLLLEMLTFNPHKRISAFRALQHSYLHKDEGNPEGGSGGGGSGLNDIFEAQKIEWHE

SEQ ID NO: 6 - protein, 686 aa, synthetic construct
MHHHHHHHHHMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSENLYFQGGSGMEHQLLCCEVETIRRAYPDANLLNDRVLRAMLKAEETCAPSVSYFKCVQKEVLPSMRKIVATWMLEVCEEQKCEEEVFPLAMNYLDRFLSLEPVKKSRLQLLGATCMFVASKMKETIPLTAEKLCIYTDNSIRPEELLQMELLLVNKLKWNLAAMTPHDFIEHFLSKMPEAEENKQIIRKHAQTFVALCATDVKFISNPPSMVAAGSVVAAVQGLNLRSPNNFLSYYRLTRFLSRVIKCDPDCLRACQEQIEALLESSLRQAQQNMDPKAAEEEEEEEEEVDLACTPTDVRDVDI

SEQ ID NO: 7 - protein, 746 aa, synthetic construct
MHHHHHHHHHMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSMMGSSSSGLEVLFQGPMEKDGLCRADQQYECVAEIGEGAYGKVFKARDLKNGGRFVALKRVRVQTGEEGMPLSTIREVAVLRHLETFEHPNVVRLFDVCTVSRTDRETKLTLVFEHVDQDLTTYLDKVPEPGVPTETIKDMMFQLLRGLDFLHSHRVVHRDLKPQNILVTSSGQIKLADFGLARIYSFQMALTSVVVTLWYRAPEVLLQSSYATPVDLWSVGCIFAEMFRRKPLFRGSSDVDQLGKILDVIGLPGEEDWPRDVALPRQAFHSKSAQPIEKFVTDIDELGKDLLLKCLTFNPAKRISAYSALSHPYFQDLERCKENLDSHLPPSQNTSELNTAGGSGGGGSGLNDIFEAQKIEWHE

SEQ ID NO: 8 - protein, 682 aa, synthetic construct
MHHHHHHHHHMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSLEVLQGPGSMELLCCEGTRHAPRAGPDPRLLGDQRVLQSLLRLEERYVPRASYFQCVQREIKPHMRKMLAYWMLEVCEEQRCEEEVFPLAMNYLDRYLSCVPTRKAQLQLLGAVCMLLASKLRETTPLTIEKLCIYTDHAVSPRQLRDWEVLVLGKLKWDLAAVIAHDFLAFILHRLSLPRDRQALVKKHAQTFLALCATDYTFAMYPPSMIATGSIGAAVQGLGACSMSGDELTELLAGITGTEVDCLRACQEQIEAALRESLREASQTSSSPAPKAPRGSSSQGPSQTSTPTDVTAIHL

SEQ ID NO: 9 - protein, 564 aa, synthetic construct
MHHHHHHHHHMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSENLYFQGGSTRPPTLSPIPHIPRSPYKFPSSPLRIPGGNIYISPLKSPYKISEGLPTPTKMTPRSRILVSIGESFGTSEKFQKINQMVCNSDRVLKRSAEGSNPPKPLKKLRFDIEGSDEADGSKHLPGESKFQQKLAEMTSTRTRMQKQKMNDSMDTSNGGSGGGGSGLNDIFEAQKIEWHE

SEQ ID NO: 10 - protein, 15 aa, synthetic construct
GLNDIFEAQKIEWHE

SEQ ID NO: 11 - protein, 8 aa, synthetic construct
GGSGGGGS

SEQ ID NO: 12 - protein, 23 aa, synthetic construct
GGSGGGGSGLNDIFEAQKIEWHE

(19) United States
(12) Patent Application Publication
(10) Pub. No.: US 2024/0360137 A1
(43) Pub. Date: Oct. 31, 2024

(54) CYCLIN-DEPENDENT KINASE (CDK2) INHIBITORS

(71) Applicant: Novartis AG, Basel (CH)

(72) Inventors: Karin BRINER, Cambridge, MA (US); Xin CHEN, Lexington, MA (US); Sajan JOSEPH, Sudbury, MA (US); Vivek RAUNIYAR, Cambridge, MA (US); Frédéric ZECRI, Brookline, MA (US)

(21) Appl. No.: 18/442,235

(22) Filed: Feb. 15, 2024

Related U.S. Application Data
(60) Provisional application No. 63/580,277, filed on Sep. 1, 2023.
(60) Provisional application No. 63/485,709, filed on Feb. 17, 2023.

(51) Int. Cl. C07D 487/04; A61K 31/519; C07D 519/00

(52) U.S. Cl. CPC C07D 487/04; A61K 31/519; C07D 519/00

(57) ABSTRACT

The invention relates to compounds which inhibit CDK2 (Cyclin-Dependent Kinase 2 or Cell Division protein Kinase 2), and to processes for the preparation of said compounds, pharmaceutical compositions comprising said compounds, and use of said compounds in the treatment of conditions, diseases and disorders mediated by CDK2.

### FIELD OF THE INVENTION

The invention relates to compounds which inhibit CDK2 (Cyclin-Dependent Kinase 2 or Cell Division protein Kinase 2), and to processes for the preparation of said compounds, pharmaceutical compositions comprising said compounds, and use of said compounds in the treatment of conditions, diseases and disorders mediated by CDK2.

### SEQUENCE LISTING

The instant application contains a Sequence Listing, which has been submitted electronically in XML format and is hereby incorporated by reference in its entirety. Said XML copy, created on Sep. 1, 2023 is named PAT059456-WO-PCT_ST26 SQL.xml and is 17,780 bytes in size.

### BACKGROUND OF THE INVENTION

CDK2 is a serine/threonine kinase that regulates the mammalian cell cycle. After binding to cyclin-E1, cyclin-E2, cyclin-A1 or cyclin-A2, the activated CDK2-cyclin complex phosphorylates a variety of cellular substrates (Chi et al 2020). For example, it phosphorylates and inactivates the retinoblastoma protein (pRB), thus de-represses the E2F family of transcription factors that promotes expression of cell cycle related genes (Sherr & Roberts 1999, 2004). As such, CDK2 propels the cell from the G1 phase into S phase of the cell cycle, when nuclear DNA is replicated in preparation of mitosis. CDK2 and its activity are often dysregulated in human cancers (Corsino et al 2008, Ying et al 2018, Nie et al 2019). One such dysregulation mechanism involves, but not limited to, the amplification or overexpression of cyclin-E1, which leads to hyperactivation of CDK2 and promotes cellular proliferation (Schraml et al. 2003). Depending on lineage, dysregulation of CDK2/E occurs in up to 40% of human cancers (cbioportal.org), including but not limited to uterine carcinosarcoma (˜40%) ovarian (˜20%), gastric (˜12%), esophageal (˜10%), endometrial (˜10%), and breast cancers (˜10%). Cyclin-E1 amplification and overexpression is also associated with poor prognosis across cancers (Nakayama et al 2010, Zhao et al 2019). In many of these cancers, besides radiation and chemotherapy, there is no targeted therapy available to patients. Thus, the development of a targeted agent against CDK2 would potentially benefit cancer patients.
Chi Y., Carter J. H, Swanger J., Mazin A. V., Moritz R. L, and Clurman B. E. (2020). A novel landscape of nuclear human CDk2 substrates revelaed by in situ phosphorylation. Sci. Adv 6, eaaz9899
Sherr C J, Roberts J M. (1999). CDK inhibitors: positive and negative regulators of G1-phase progression. Genes Dev 13: 1501-1512.
Sherr C J, Roberts J M. (2004). Living with or without cyclins and cyclin-dependent kinases. Genes Dev 18: 2699-2711.
Corsino P E, Davis B J, Norgaard P H, Parker N N, Law M, Dunn W and Law B K. Mammary tumors initiated by constitutive Cdk2 activation contain an invasive basal-like component. Neoplasia 2008; 10: 1240-1252
Ying M, Shao X, Jing H, Liu Y, Qi X, Cao J, et al. Ubiquitin-dependent degradation of CDK2 drives the therapeutic differentiation of AML by targeting PRDX2. Blood J Am Soc Hematol. 2018; 131(24):2698-711
Nie L, Wei Y, Zhang F, Hsu Y—H, Chan L-C, Xia W, et al. CDK2-mediated site-specific phosphorylation of EZH2 drives and maintains triplenegative breast cancer. Nat Commun. 2019; 10(1):1-15
Schraml P, Bucher C, Bissig H, et al. Cyclin E overexpression and amplification in human tumours. J Pathol. 2003; 200(3):375-383.
Nakayama N, Nakayama K, Shamima Y, Ishikawa M, Katagiri A, lida K, et al. Gene amplification CCNE1 is related to poor survival and potential therapeutic target in ovarian cancer. Cancer 2010:NA-NA.
Guo Q, Lin X, Ye L, et al. Comparative efficacy of Cdk4/6 inhibitors plus aromatase inhibitors versus fulvestrant for the first-line treatment of hormone receptor-positive advanced breast cancer: a network meta-analysis. Target Oncol 2019; 14:139-48
Gao J J, Cheng J, Bloomquist E, et al. Cdk4/6 inhibitor treatment for patients with hormone receptor-positive, HER2-negative, advanced or metastatic breast cancer: a US food and drug administration pooled analysis. Lancet Oncol 2020; 21:250-60

WO2020/168178 is directed to various CDK2 inhibitors structurally related to those disclosed herein. For example, compounds such as

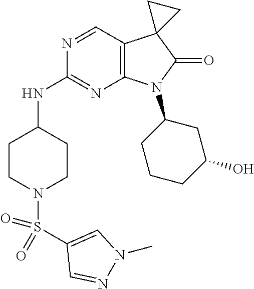 [SMILES: Cn1cc(S(=O)(=O)N2CCC(Nc3ncc4c(n3)N([C@@H]3CCC[C@@H](O)C3)C(=O)C43CC3)CC2)cn1]

### (Example 12).

WO2023/278326 (Blueprint) discloses compounds such as

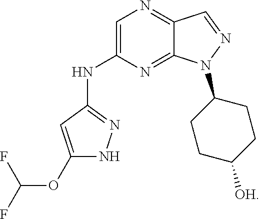 [SMILES: O[C@H]1CC[C@H](n2ncc3ncc(Nc4cc(OC(F)F)[nH]n4)nc32)CC1]

Notably, the Blueprint compounds bind to the CDK2 ATP-binding site in a very different manner to the Incyte compounds, as shown in FIG. 1 and the associated brief description.

### SUMMARY OF INVENTION

The applicant has surprisingly found that compounds of formula (I) as described herein, which include a(n optionally substituted) 5 membered heteroaryl in place of the bulkier

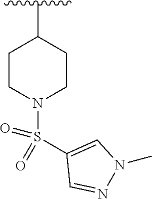 [SMILES: *C1CCN(S(=O)(=O)c2cnn(C)c2)CC1]

motif of

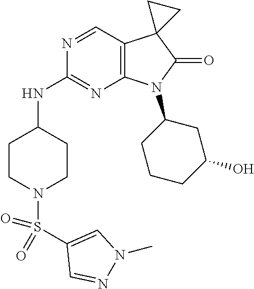 [SMILES: Cn1cc(S(=O)(=O)N2CCC(Nc3ncc4c(n3)N([C@@H]3CCC[C@@H](O)C3)C(=O)C43CC3)CC2)cn1]

(Incyte) provide a novel vector for potency and selectivity over other kinases along with optimal in-vivo PK properties.

Thus, according to a first aspect of the invention, there is hereby provided a compound according to formula (I):

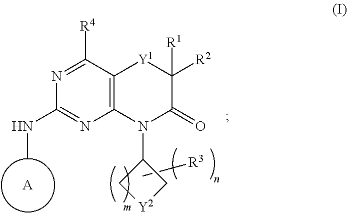

wherein:
Y¹ is a bond or CH₂ (e.g. Y¹ is a bond);
Y² is a bond, O, NR⁵ or CR⁶R⁷ (e.g. Y² is a bond);
R¹ and R² are each independently selected from the group consisting of H, halo, C₁-C₆alkyl and C₁-C₆haloalkyl, or R¹ and R² join together to form C₃-C₄cycloalkyl or C₃-C₄cyclohaloalkyl (e.g. R¹ and R² join together to form C₃-C₄cycloalkyl, e.g. R¹ and R² join together to form C₃cycloalkyl);
each R³ is independently selected from the group consisting of hydroxyl, halo, C₁-C₆alkyl and C₁-C₆haloalkyl;
R⁴ is selected from the group consisting of H, halo, C₁-C₆alkyl and C₁-C₆haloalkyl (e.g. R⁴ is H);
R⁵ is selected from the group consisting of H, C₁-C₆alkyl, C(═O)—C₁-C₆alkyl or C(═O)—O—C₁-C₆alkyl;
R⁶ and R⁷ join together to form, together with the carbon atom to which they are mutually attached, a C₃-C₆cycloalkyl or a 3-6 membered heterocyclyl comprising 1-3 heteroatoms independently selected from the group consisting of O, N and S, wherein said C₃-C₆cycloalkyl or 3-6 membered heterocyclyl is substituted with 0-3 substituents R⁸;
each R⁸ is independently selected from the group consisting of C₁-C₆alkyl, C(═O)C₁-C₆alkyl, halo, C₁-C₆haloalkyl, S—C₁-C₆alkyl, SO—C₁-C₆alkyl, SO₂—C₁-C₆alkyl, cyano, hydroxyl, or wherein two R⁸ substituents on the same ring atom join together to form ═O.
n is 0 to 3 (e.g. n is 1, e.g. n is 1 and R³ is hydroxyl);
m is 1 to 5 (e.g. m is 4);

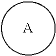

is a 5 membered heteroaryl comprising 1 to 3 heteroatoms independently selected from N, O and S (e.g. 2 heteroatoms independently selected from N, O and S, e.g. 2 heteroatoms which are both N), said 5 membered heteroaryl being substituted with 0 to 3 substituents R^{A}.
each R^{A} is independently *L¹-X¹, wherein * indicates the point of attachment to

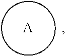

each L¹ is independently selected from bond, O, S, SO, SO₂, C≡C, C(═O), *C(═O)—O**, C₁-C₆alkylene, C₁-C₆haloalkylene, *O—C₁-C₆alkylene**, *O—C₁-C₆haloalkylene**, *O—C₁-C₆hydroxyalkylene**, C₁-C₆alkylene-O—C₁-C₆alkylene, *O—C₃-C₆cycloalkylene**, *O-3-6 membered heterocyclylene**, C₁-C₆hydroxyalkylene, C₃-C₆cycloalkylene, 3-6 membered heterocyclylene (e.g. comprising 1 heteroatom which is O), O—C₁-C₆alkylene-O, *O—C₁-C₆alkylene-O—C₃-C₆cycloalkylene** and *O-C₁-C₆alkylene-O-3-6 membered heterocyclylene**, wherein * indicates the point of attachment to

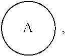

and ** indicates the point of attachment to X¹;
and each X¹ is independently selected from H, halo, cyano, hydroxyl, C₁-C₆alkyl, C(═O)—C₁-C₆alkyl, C₁-C₆haloalkyl, C₃-C₆cycloalkyl substituted by 0-3 R³ groups, O—C₁-C₆alkyl, S—C₁-C₆alkyl, S(O)—C₁-C₆alkyl, S(O)₂—C₁-C₆alkyl, N(C₁-C₆alkyl)₂, C(═O)N(C₁-C₆alkyl)₂, C₁-C₆hydroxyalkyl, 3-6 membered heterocyclyl (e.g. comprising 1 or 2 heteroatoms independently selected from O, N and S) substituted by 0-3 R³ groups, 5-10 membered heteroaryl (e.g. 5, 6, 9 or 10 membered heteroaryl) comprising 1-4 heteroatoms independently selected from O, N and S substituted by 0-3 R³ groups, 5-10 membered partially saturated heterocyclyl comprising 1 to 4 heteroatoms independently selected from the group consisting of O, N and S substituted by 0-3 R³ groups, 7-10 membered spiroheterocyclyl comprising 1 or 2 heteroatoms independently selected from O, N and S substituted by 0-3 R³ groups and C₇-C₁₀spirocycloalkyl substituted by 0-3 R³ groups;
or
two R^{A} substituents located on adjacent ring atoms join together to form with said adjacent ring atoms a 4 to 6 membered heterocyclyl comprising 1 to 3 heteroatoms independently selected from N, O and S, with the proviso that at least one heteroatom is N;
or a pharmaceutically acceptable salt and/or tautomer thereof.

According to a second aspect of the invention, there is hereby provided a compound selected from any one of the Examples or any one of the synthetic intermediates, or a pharmaceutically acceptable salt and/or tautomer thereof.

According to a third aspect of the invention, there is hereby provided a pharmaceutical composition comprising the compound or pharmaceutically acceptable salt and/or tautomer thereof according to the first or the second aspect of the invention and one or more pharmaceutically acceptable carriers.

According to a fourth aspect of the invention, there is hereby provided a combination comprising the compound or pharmaceutically acceptable salt and/or tautomer thereof according to the first or the second aspect of the invention, and one or more therapeutically active agents.

According to a fifth aspect of the invention, there is hereby provided a method of modulating CDK2 activity in a subject comprising administering to the subject a therapeutically effective amount of the compound or pharmaceutically acceptable salt and/or tautomer thereof according to the first or the second aspect of the invention.

According to a sixth aspect of the invention, there is hereby provided a method of treating cancer comprising administering to a subject in need thereof a therapeutically effective amount of the compound or pharmaceutically acceptable salt and/or tautomer thereof according to the first or the second aspect of the invention.

According to a seventh aspect of the invention, there is hereby provided a compound or pharmaceutically acceptable salt and/or tautomer thereof according to the first or the second aspect of the invention for use as a medicament.

According to a eighth aspect of the invention, there is hereby provided a compound or pharmaceutically acceptable salt and/or tautomer thereof according to the first or the second aspect of the invention for use in the treatment of cancer.

According to a ninth aspect of the invention, there is hereby provided use of the compound or pharmaceutically acceptable salt and/or tautomer thereof according to the first or the second aspect of the invention in the treatment of cancer.

According to a tenth aspect of the invention, there is hereby provided use of the compound or pharmaceutically acceptable salt and/or tautomer thereof according to the first or the second aspect of the invention in the manufacture of a medicament for the treatment of cancer.

### BRIEF DESCRIPTION OF THE DRAWINGS

FIG. 1 shows (top) the putative binding modes of

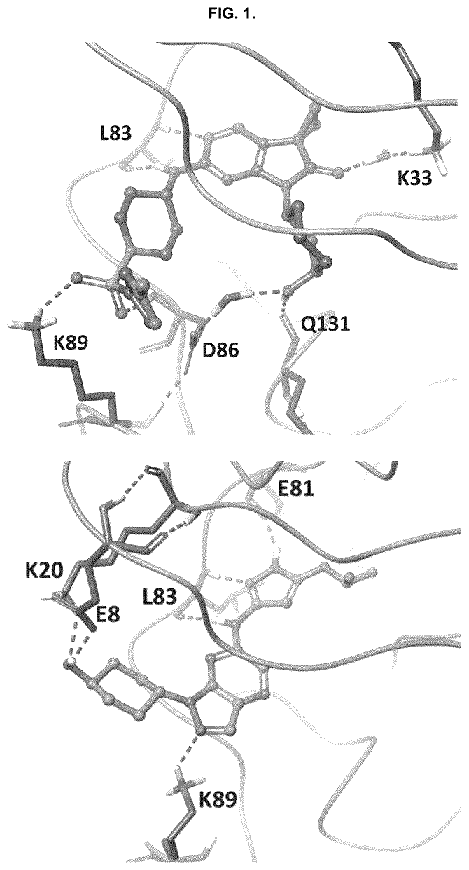

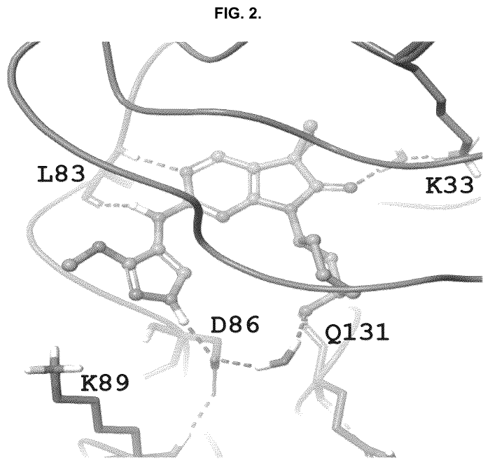

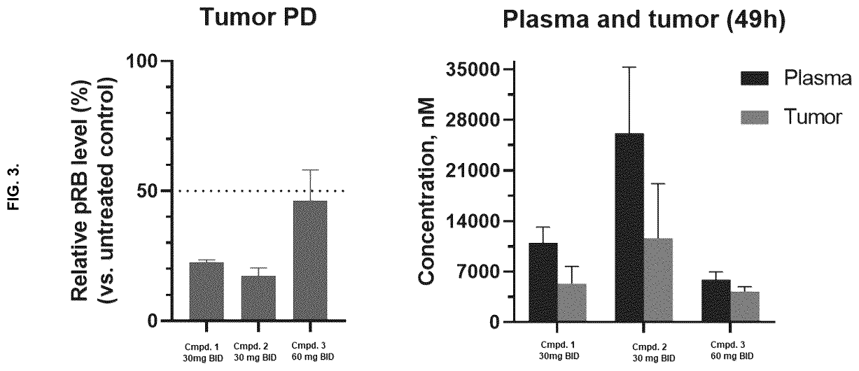

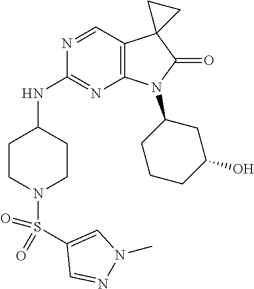 [SMILES: Cn1cc(S(=O)(=O)N2CCC(Nc3ncc4c(n3)N([C@@H]3CCC[C@@H](O)C3)C(=O)C43CC3)CC2)cn1]

(Incyte) and (bottom)

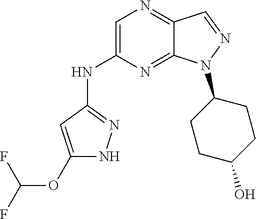 [SMILES: O[C@H]1CC[C@H](n2ncc3ncc(Nc4cc(OC(F)F)[nH]n4)nc32)CC1]

(Blueprint) to the CDK2 ATP-binding site. These putative binding modes were modeled by the molecular modeling software package Maestro® (Schrodinger, LLC).

The Incyte compound forms two hydrogen bonds with the hinge region of CDK2 (L83). It also forms a hydrogen bond with the catalytic K33 in the rear. In contrast, the blueprint compound forms three hydrogen bonds with the hinge region of CDK2 (L83 and E81), and it doesn't form any polar interactions in the rear, including at K33. Instead, it forms two different hydrogen bonds with residues at the front (K₂O and E8). In conclusion, the binding modes of the Incyte and Blueprint compounds to the CDK2 ATP-binding site are very different to one another.

FIG. 2 shows the putative binding mode of a compound according to formula (I). The binding is broadly similar to

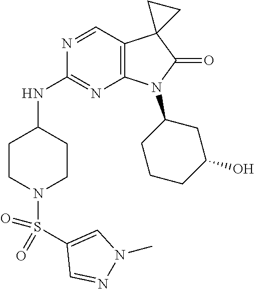 [SMILES: Cn1cc(S(=O)(=O)N2CCC(Nc3ncc4c(n3)N([C@@H]3CCC[C@@H](O)C3)C(=O)C43CC3)CC2)cn1]

(Incyte), but very different to that of

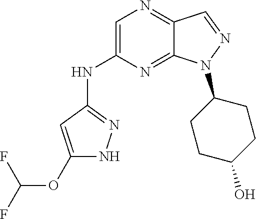 [SMILES: O[C@H]1CC[C@H](n2ncc3ncc(Nc4cc(OC(F)F)[nH]n4)nc32)CC1]

(Blueprint). These putative binding modes were modeled by the molecular modeling software package Maestro® (Schrodinger, LLC).

FIG. 3 shows the impact of 3 days of oral BID dosing with indicated compounds on PD marker (RB phosphorylation) in the Cyclin E amplified OVCAR3 xenograft model. Data is shown as percentage of untreated animals.

Compound 1=

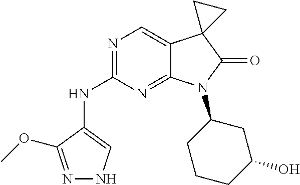 [SMILES: COc1n[nH]cc1Nc1ncc2c(n1)N([C@@H]1CCC[C@@H](O)C1)C(=O)C21CC1]

Compound 2=

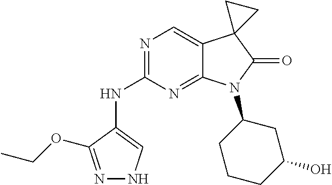 [SMILES: CCOc1n[nH]cc1Nc1ncc2c(n1)N([C@@H]1CCC[C@@H](O)C1)C(=O)C21CC1]

Compound 3=

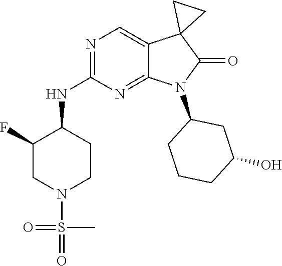 [SMILES: CS(=O)(=O)N1CC[C@H](Nc2ncc3c(n2)N([C@@H]2CCC[C@@H](O)C2)C(=O)C32CC2)[C@H](F)C1]

### DETAILED DESCRIPTION OF THE INVENTION

The invention therefore, in a first aspect, provides a compound according to formula (I):

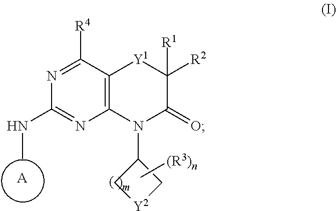

wherein:
Y¹ is a bond or CH₂ (e.g. Y¹ is a bond);
Y² is a bond, O, NR⁵ or CR⁶R⁷ (e.g. Y² is a bond);
R¹ and R² are each independently selected from the group consisting of H, halo, C₁-C₆alkyl and C₁-C₆haloalkyl, or R¹ and R² join together to form C₃-C₄cycloalkyl or C₃-C₄cyclohaloalkyl (e.g. R¹ and R² join together to form C₃-C₄cycloalkyl, e.g. R¹ and R² join together to form C₃cycloalkyl);
each R³ is independently selected from the group consisting of hydroxyl, halo, C₁-C₆alkyl and C₁-C₆haloalkyl;
R⁴ is selected from the group consisting of H, halo, C₁-C₆alkyl and C₁-C₆haloalkyl (e.g. R⁴ is H);
R⁵ is selected from the group consisting of H, C₁-C₆alkyl, C(═O)—C₁-C₆alkyl or C(═O)—O—C₁-C₆alkyl;
R⁶ and R⁷ join together to form, together with the carbon atom to which they are mutually attached, a C₃-C₆cycloalkyl or a 3-6 membered heterocyclyl comprising 1-3 heteroatoms independently selected from the group consisting of O, N and S, wherein said C₃-C₆cycloalkyl or 3-6 membered heterocyclyl is substituted with 0-3 substituents R⁸;
each R⁸ is independently selected from the group consisting of C₁-C₆alkyl, C(═O)C₁-C₆alkyl, halo, C₁-C₆haloalkyl, S—C₁-C₆alkyl, SO—C₁-C₆alkyl, SO₂—C₁-C₆alkyl, cyano, hydroxyl, or wherein two R⁸ substituents on the same ring atom join together to form ═O.
n is 0 to 3 (e.g. n is 1, e.g. n is 1 and R³ is hydroxyl);
m is 1 to 5 (e.g. m is 4);

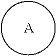

is a 5 membered heteroaryl comprising 1 to 3 heteroatoms independently selected from N, O and S (e.g. 2 heteroatoms independently selected from N, O and S, e.g. 2 heteroatoms which are both N), said 5 membered heteroaryl being substituted with 0 to 3 substituents R^{A}.
each R^{A} is independently *L¹-X¹, wherein * indicates the point of attachment to

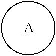

each L¹ is independently selected from bond, O, S, SO, SO₂, C≡C, C(═O), *C(═O)—O**, C₁-C₆alkylene, C₁-C₆haloalkylene, *O—C₁-C₆alkylene**, *O—C₁-C₆haloalkylene**, *O—C₁-C₆hydroxyalkylene**, C₁-C₆alkylene-O—C₁-C₆alkylene, *O—C₃-C₆cycloalkylene**, *O-3-6 membered heterocyclylene**, C₁-C₆hydroxyalkylene, C₃-C₆cycloalkylene, 3-6 membered heterocyclylene (e.g. comprising 1 heteroatom which is O), O—C₁-C₆alkylene-O, *O—C₁-C₆alkylene-O—C₃-C₆cycloalkylene** and *O—C₁-C₆alkylene-O-3-6 membered heterocyclylene**, wherein * indicates the point of attachment to

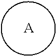

and ** indicates the point of attachment to X¹;
and each X¹ is independently selected from H, halo, cyano, hydroxyl, C₁-C₆alkyl, C(═O)—C₁-C₆alkyl, C₁-C₆haloalkyl, C₃-C₆cycloalkyl substituted by 0-3 R³ groups, O—C₁-C₆alkyl, S—C₁-C₆alkyl, S(O)—C₁-C₆alkyl, S(O)₂—C₁-C₆alkyl, N(C₁-C₆alkyl)₂, C(═O)N(C₁-C₆alkyl)₂, C₁-C₆hydroxyalkyl, 3-6 membered heterocyclyl (e.g. comprising 1 or 2 heteroatoms independently selected from O, N and S) substituted by 0-3 R³ groups, 5-10 membered heteroaryl (e.g. 5, 6, 9 or 10 membered heteroaryl) comprising 1-4 heteroatoms independently selected from O, N and S substituted by 0-3 R³ groups, 5-10 membered partially saturated heterocyclyl comprising 1 to 4 heteroatoms independently selected from the group consisting of O, N and S substituted by 0-3 R³ groups, 7-10 membered spiroheterocyclyl comprising 1 or 2 heteroatoms independently selected from O, N and S substituted by 0-3 R³ groups and C₇-C₁₀spirocycloalkyl substituted by 0-3 R³ groups; or
two R^{A} substituents located on adjacent ring atoms join together to form with said adjacent ring atoms a 4 to 6 membered heterocyclyl comprising 1 to 3 heteroatoms independently selected from N, O and S, with the proviso that at least one heteroatom is N;
or a pharmaceutically acceptable salt and/or tautomer thereof.

In relation to the L¹ groups defined above, it should be understood that for certain groups such as C₁-C₆alkylene and O—C₁-C₆alkylene-O* and ** are undefined because the substituent is symmetrical i.e. *C₁-C₆alkylene** is the same as **C₁-C₆alkylene*.

In an embodiment, Y¹ is a bond.

In an alternative (less favored) embodiment, Y¹ is CH₂ and R² is H.

In an embodiment, Y² is a bond.

In an embodiment, m is 4.

In an embodiment, n is 1 to 3, e.g. n is 1.

In an embodiment, at least one R³ is OH.

In an embodiment, the compound of formula (I) is a compound of formula (Ia):

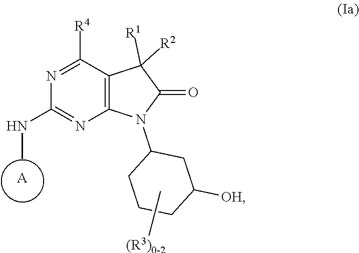

wherein R¹, R², R³, R⁴ and

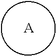

are as defined above.

In an embodiment, the compound of formula (I) is a compound of formula (Ib):

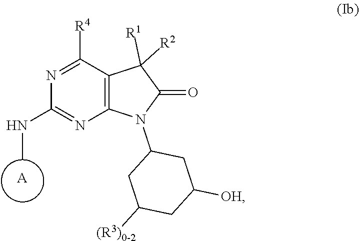

wherein R¹, R², R³, R⁴ and

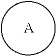

are as defined above.

In an embodiment, the compound of formula (I) is a compound of formula (Ic):

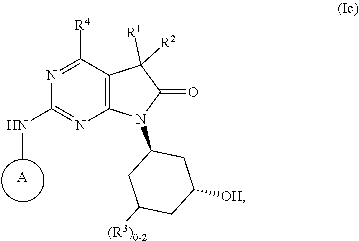

wherein R¹, R², R³, R⁴ and

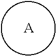

are as defined above.

In an embodiment, the compound of formula (I) is a compound of formula (Id):

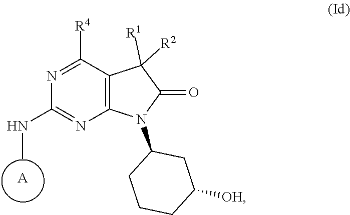

wherein R¹, R², R⁴ and

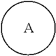

are as defined above.

In an embodiment, R¹ and R² join together to form C₃-C₄cycloalkyl or C₃-C₄cyclohaloalkyl.

In an embodiment, R¹ and R² join together to form C₃-C₄cycloalkyl.

In an embodiment, R¹ and R² join together to form C₃cycloalkyl.

In an embodiment, R⁴ is H.

In an embodiment, R¹ and R² join together to form C₃-C₄cycloalkyl, and R⁴ is H.

In an embodiment, R¹ and R² join together to form C₃cycloalkyl, and R⁴ is H.

In an embodiment, m is 4 and R¹ and R² join together to form C₃-C₄cycloalkyl.

In an embodiment, m is 4 and R¹ and R² join together to form C₃cycloalkyl.

In an embodiment, m is 4 and R⁴ is H.

In an embodiment, m is 4, R¹ and R² join together to form C₃-C₄cycloalkyl and R⁴ is H.

In an embodiment, m is 4, R¹ and R² join together to form C₃cycloalkyl and R⁴ is H.

In an embodiment, Y¹ is a bond, R¹ and R² join together to form C₃-C₄cycloalkyl, and R⁴ is H.

In an embodiment, Y¹ is a bond, R¹ and R² join together to form C₃cycloalkyl, and R⁴ is H.

In an embodiment, Y¹ is a bond, m is 4 and R¹ and R² join together to form C₃-C₄cycloalkyl.

In an embodiment, Y¹ is a bond, m is 4 and R¹ and R² join together to form C₃cycloalkyl.

In an embodiment, Y¹ is a bond, m is 4 and R⁴ is H.

In an embodiment, Y¹ is a bond, m is 4, R¹ and R² join together to form C₃-C₄cycloalkyl and R⁴ is H.

In an embodiment, Y¹ is a bond, m is 4, R¹ and R² join together to form C₃cycloalkyl and R⁴ is H.

In an embodiment, Y² is a bond, R¹ and R² join together to form C₃-C₄cycloalkyl, and R⁴ is H.

In an embodiment, Y² is a bond, R¹ and R² join together to form C₃cycloalkyl, and R⁴ is H.

In an embodiment, Y² is a bond, m is 4 and R¹ and R² join together to form C₃-C₄cycloalkyl.

In an embodiment, Y² is a bond, m is 4 and R¹ and R² join together to form C₃cycloalkyl.

In an embodiment, Y² is a bond, m is 4 and R⁴ is H.

In an embodiment, Y² is a bond, m is 4, R¹ and R² join together to form C₃-C₄cycloalkyl and R⁴ is H.

In an embodiment, Y² is a bond, m is 4, R¹ and R² join together to form C₃cycloalkyl and R⁴ is H.

In an embodiment, Y¹ is a bond, Y² is a bond, R¹ and R² join together to form C₃-C₄cycloalkyl, and R⁴ is H.

In an embodiment, Y¹ is a bond, Y² is a bond, R¹ and R² join together to form C₃cycloalkyl, and R⁴ is H.

In an embodiment, Y¹ is a bond, Y² is a bond, m is 4 and R¹ and R² join together to form C₃-C₄cycloalkyl.

In an embodiment, Y¹ is a bond, Y² is a bond, m is 4 and R¹ and R² join together to form C₃cycloalkyl.

In an embodiment, Y¹ is a bond, Y² is a bond, m is 4 and R⁴ is H.

In an embodiment, Y¹ is a bond, Y² is a bond, m is 4, R¹ and R² join together to form C₃-C₄cycloalkyl and R⁴ is H.

In an embodiment, Y¹ is a bond, Y² is a bond, m is 4, R¹ and R² join together to form C₃cycloalkyl and R⁴ is H.

In an embodiment,

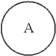

is a 5 membered heteroaryl comprising 2 heteroatoms independently selected from N, O and S, said 5 membered heteroaryl being substituted with 0 to 3 substituents R^{A}, wherein R^{A} is as defined above.

In an embodiment,

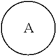

is a 5 membered heteroaryl comprising 2 heteroatoms independently selected from N, O and S, said 5 membered heteroaryl being substituted with 0 to 3 substituents R^{A}, wherein R^{A} is as defined above, Y¹ is a bond, Y² is a bond, m is 4, R¹ and R² join together to form C₃cycloalkyl and R⁴ is H.

In an embodiment,

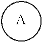

is a 5 membered heteroaryl comprising 2 heteroatoms independently selected from N and O, said 5 membered heteroaryl being substituted with 0 to 3 substituents R^{A}, wherein R^{A} is as defined above.

In an embodiment,

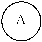

is a 5 membered heteroaryl comprising 2 heteroatoms independently selected from N and O, said 5 membered heteroaryl being substituted with 0 to 3 substituents R^{A}, wherein R^{A} is as defined above, Y¹ is a bond, Y² is a bond, m is 4, R¹ and R² join together to form C₃cycloalkyl and R⁴ is H.

In an embodiment,

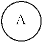

is a 5 membered heteroaryl comprising 1 to 3 heteroatoms which are each N, said 5 membered heteroaryl being substituted with 0 to 3 substituents R^{A}, wherein R^{A} is as defined above.

In an embodiment,

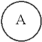

is a 5 membered heteroaryl comprising 1 to 3 heteroatoms which are each N, said 5 membered heteroaryl being substituted with 0 to 3 substituents R^{A}, wherein R^{A} is as defined above, Y¹ is a bond, Y² is a bond, m is 4, R¹ and R² join together to form C₃cycloalkyl and R⁴ is H.

In an embodiment,

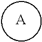

is a 5 membered heteroaryl comprising 2 heteroatoms which are each N, said 5 membered heteroaryl being substituted with 0 to 3 substituents R^{A}, wherein R^{A} is as defined above.

In an embodiment,

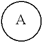

is a 5 membered heteroaryl comprising 2 heteroatoms which are each N, said 5 membered heteroaryl being substituted with 0 to 3 substituents R^{A}, wherein R^{A} is as defined above, Y¹ is a bond, Y² is a bond, m is 4, R¹ and R² join together to form C₃cycloalkyl and R⁴ is H.

In an embodiment,

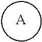

is a 5 membered heteroaryl comprising 2 heteroatoms which are each N, said 5 membered heteroaryl being substituted with 0 to 2 substituents R^{A}, wherein R^{A} is as defined above.

In an embodiment,

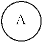

is a 5 membered heteroaryl comprising 2 heteroatoms which are each N, said 5 membered heteroaryl being substituted with 0 to 2 substituents R^{A}, wherein R^{A} is as defined above, Y¹ is a bond, Y² is a bond, m is 4, R¹ and R² join together to form C₃cycloalkyl and R⁴ is H.

In an embodiment,

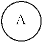

is a 5 membered heteroaryl comprising 2 heteroatoms which are each N, said 5 membered heteroaryl being substituted with 0 or 1 substituent R^{A}, wherein R^{A} is *L¹-X¹, and L¹ and X¹ are as defined above.

In an embodiment,

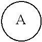

is a 5 membered heteroaryl comprising 2 heteroatoms which are each N, said 5 membered heteroaryl being substituted with 0 or 1 substituent R^{A}, wherein R^{A} is *L¹-X¹, and L¹ and X¹ are as defined above, Y¹ is a bond, Y² is a bond, m is 4, R¹ and R² join together to form C₃cycloalkyl and R⁴ is H.

In an embodiment,

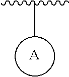

is selected from the group consisting of:

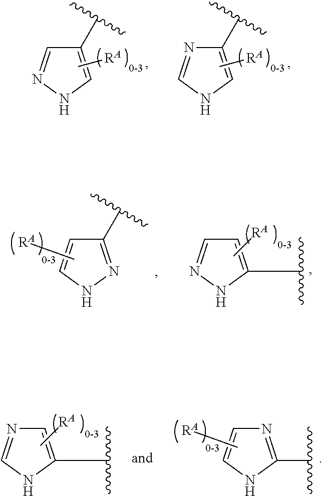

In an embodiment,

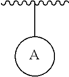

is selected from the group consisting of:

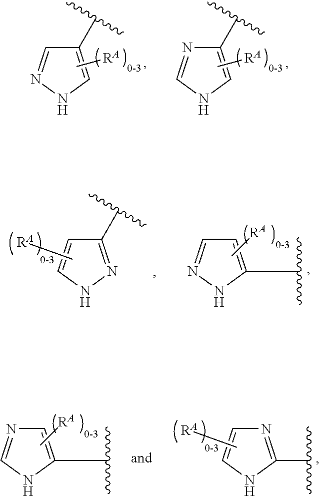

Y¹ is a bond, Y² is a bond, m is 4, R¹ and R² join together to form C₃cycloalkyl and R⁴ is H.

In an embodiment,

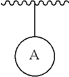

is selected from the group consisting of

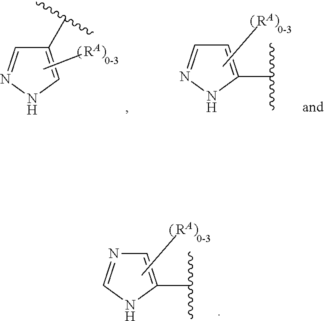

In an embodiment,

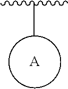

is selected from the group consisting of

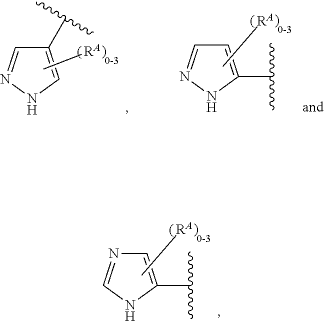

Y¹ is a bond, Y² is a bond, m is 4, R¹ and R² join together to form C₃cycloalkyl and R⁴ is H.

In an embodiment,

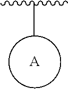

is selected from the group consisting of:

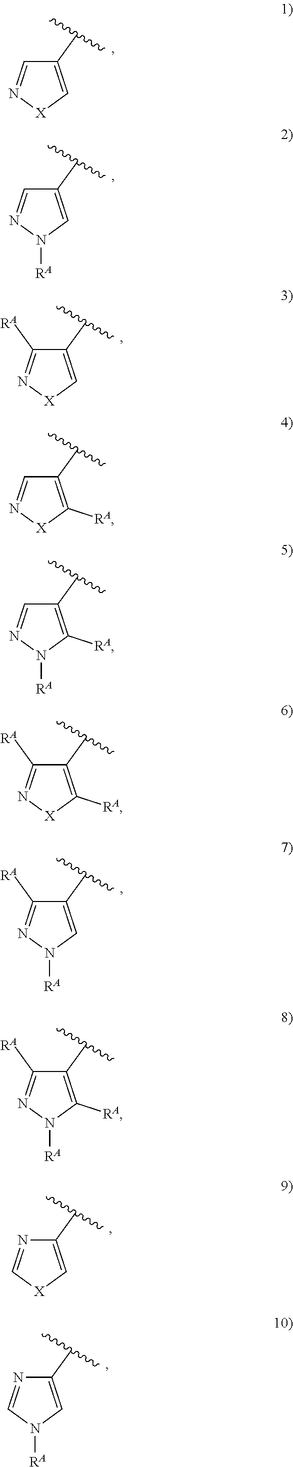

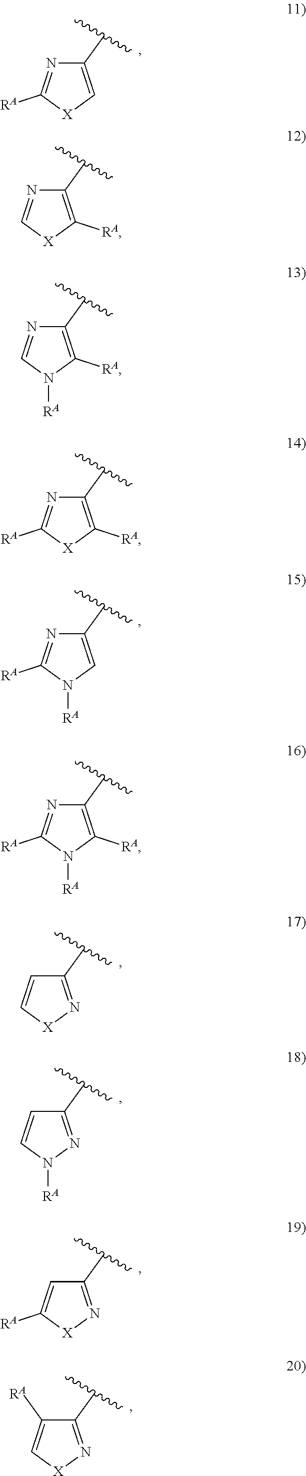

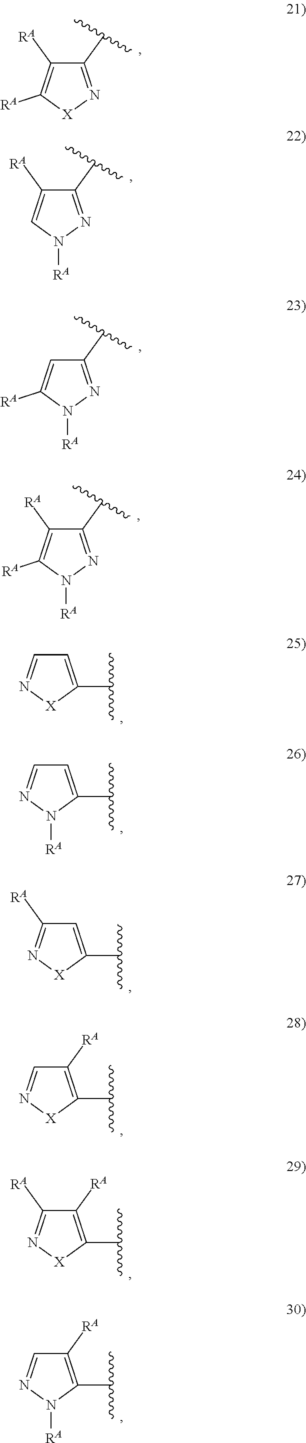

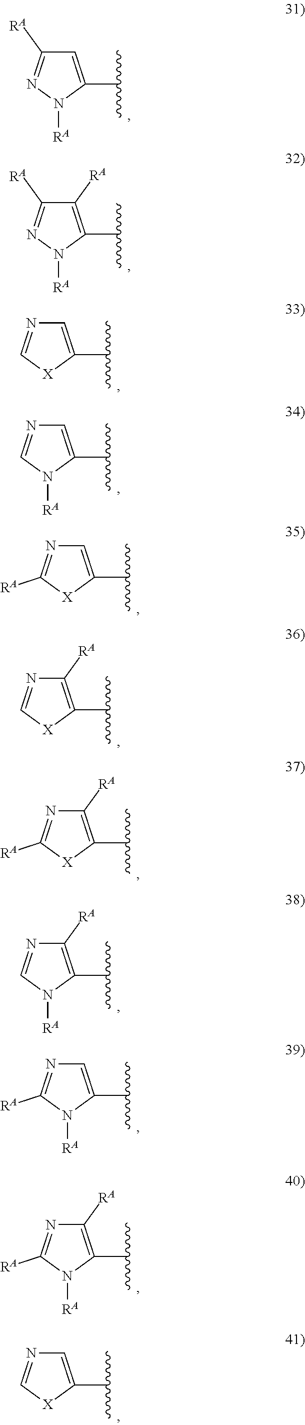

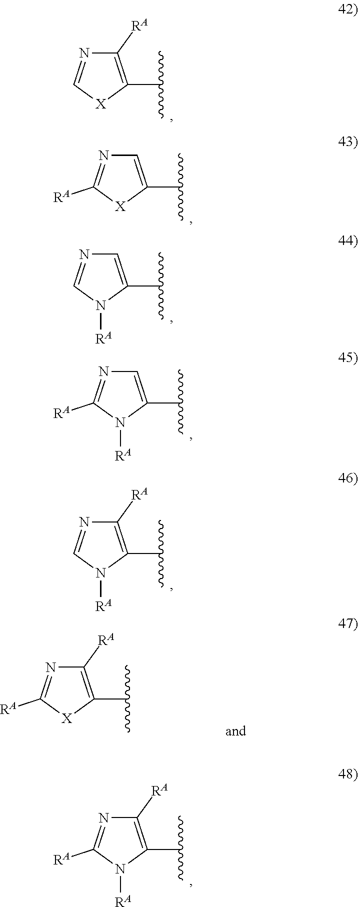

and

X is selected from O, NH and S (e.g. X is NH).

In an embodiment,

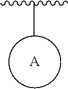

is selected from the group consisting of:

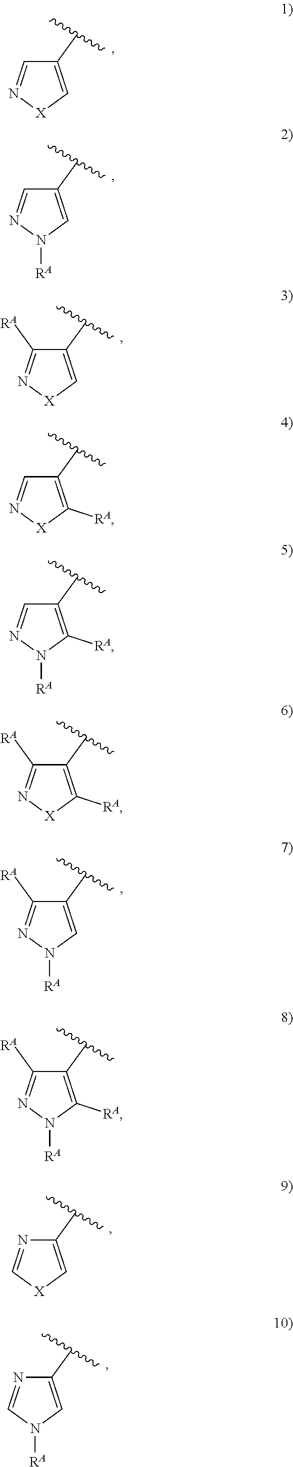

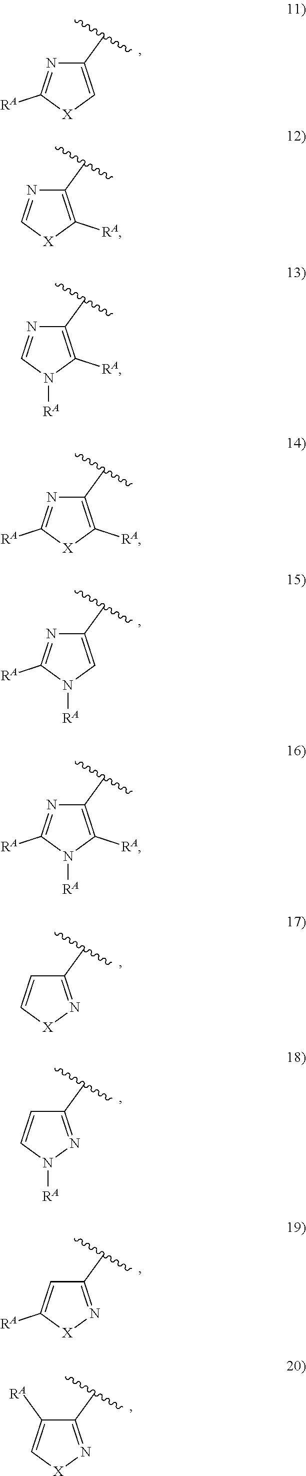

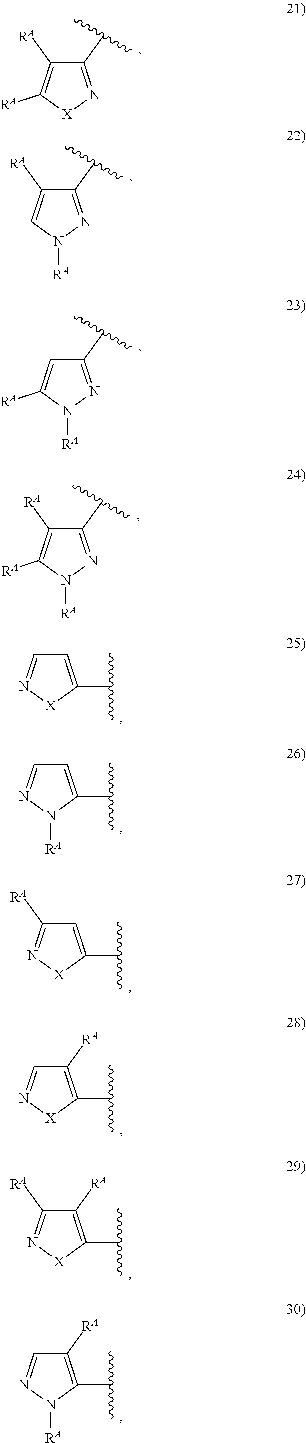

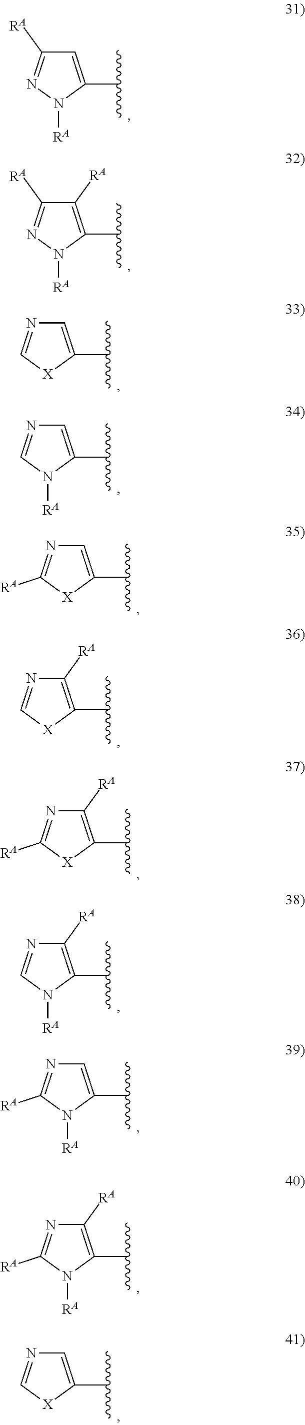

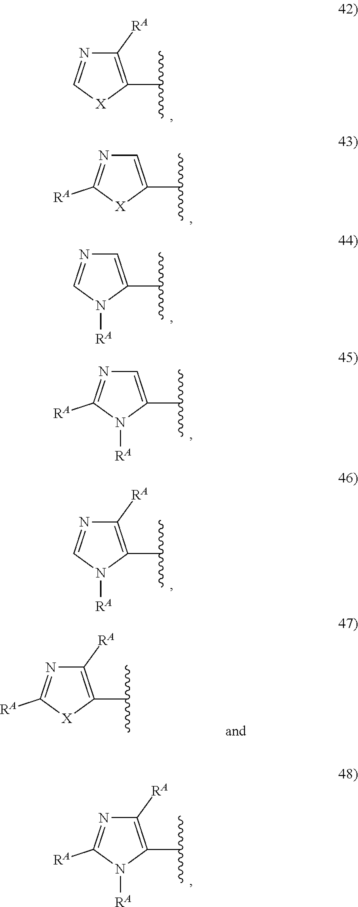

and

X is selected from O, NH and S (e.g. X is NH), Y¹ is a bond, Y² is a bond, m is 4, R¹ and R² join together to form C₃cycloalkyl and R⁴ is H.

In an embodiment,

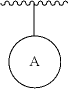

is selected from the group consisting of:

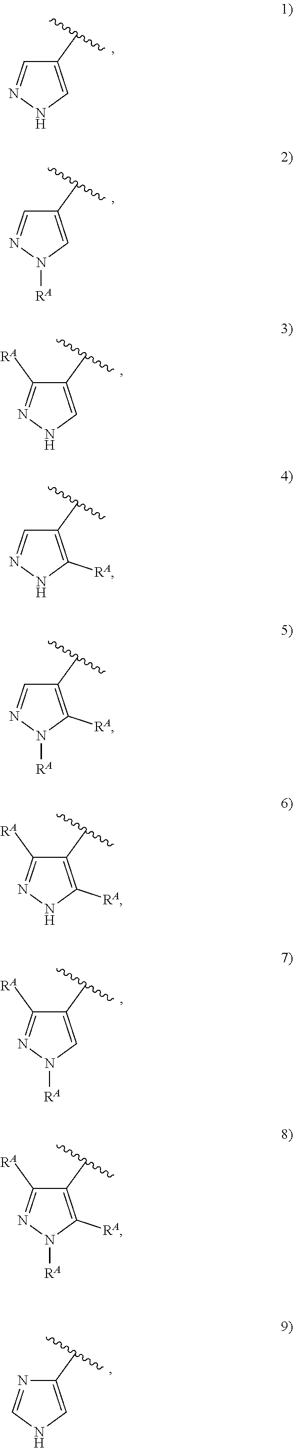

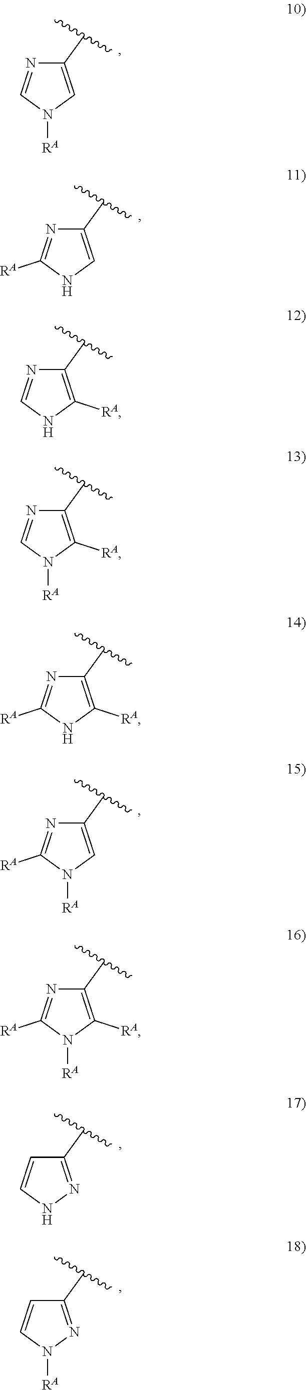

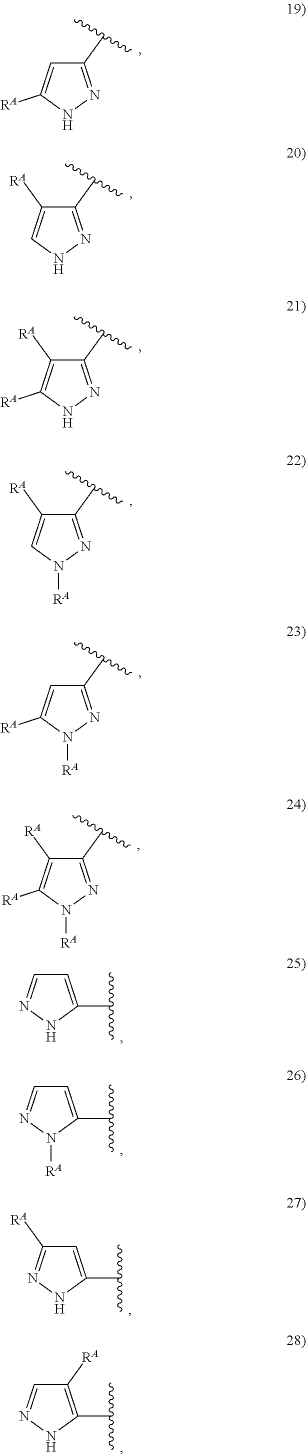

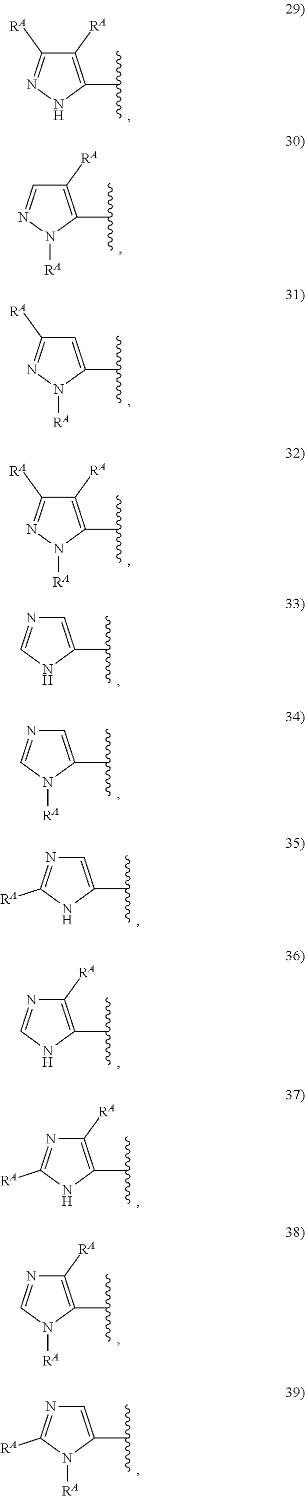

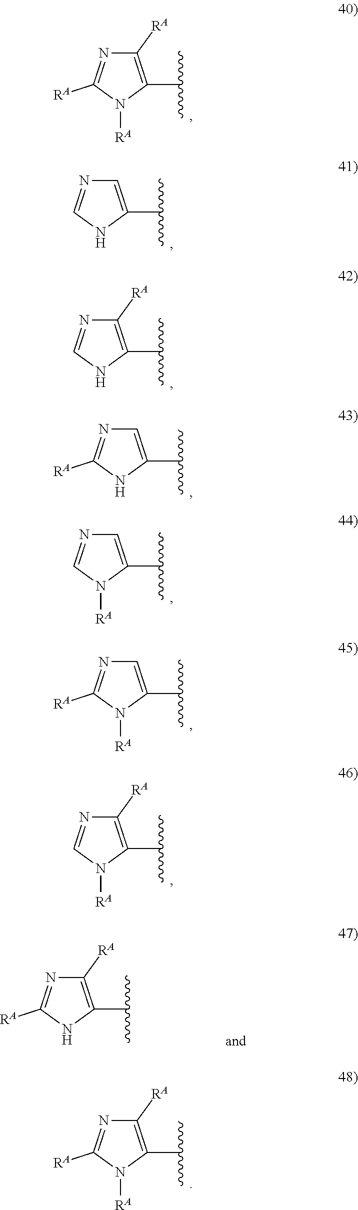

In an embodiment,

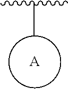

is selected from the group consisting of:

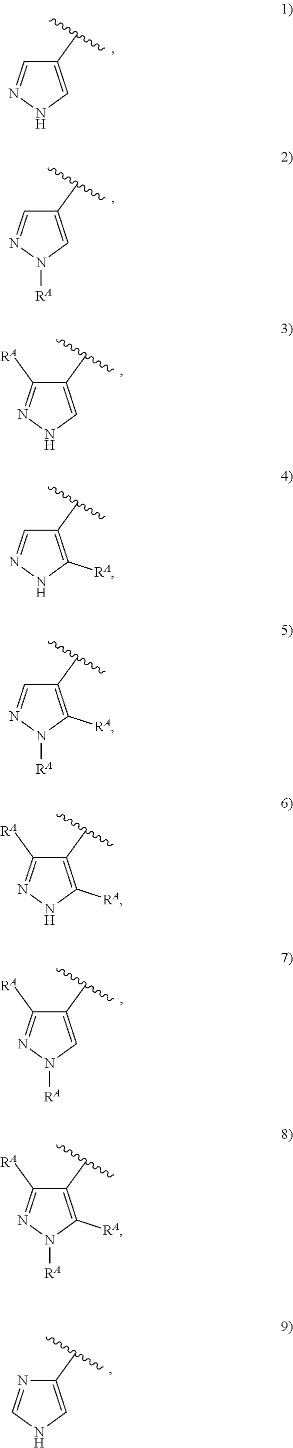

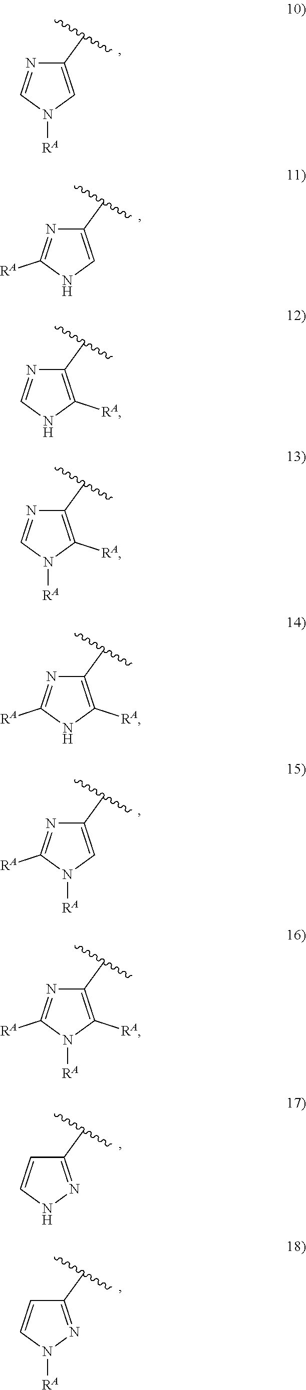

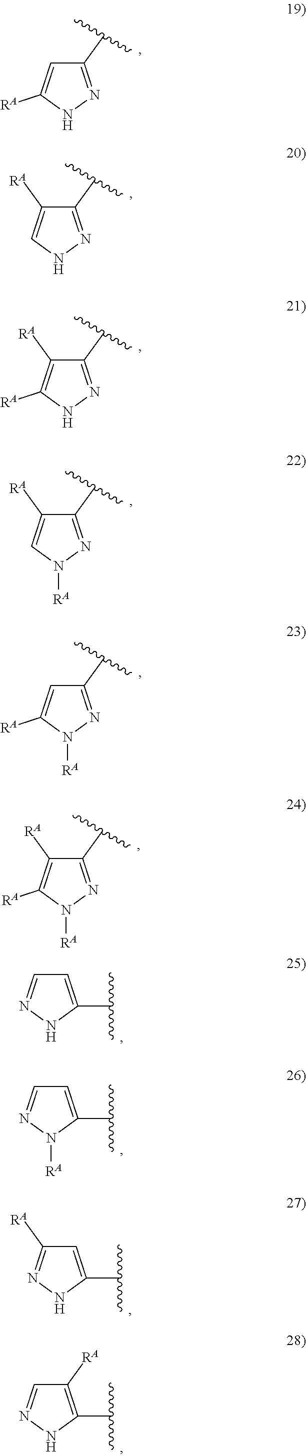

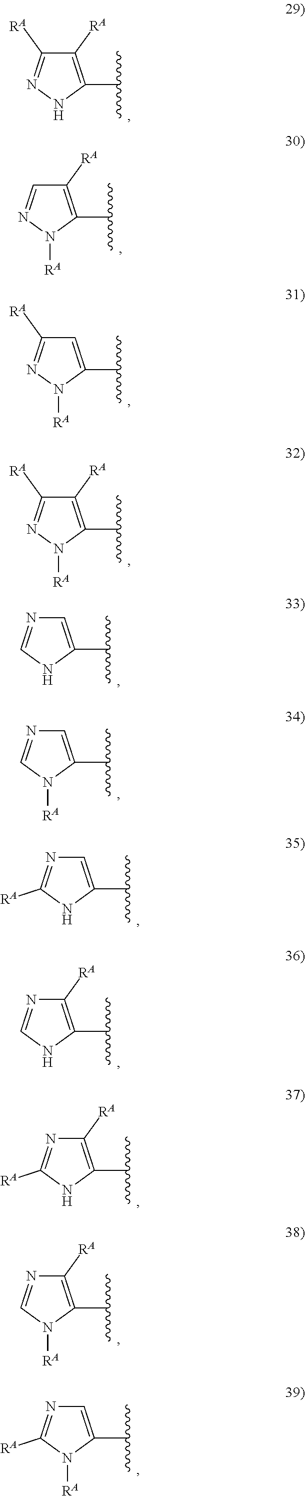

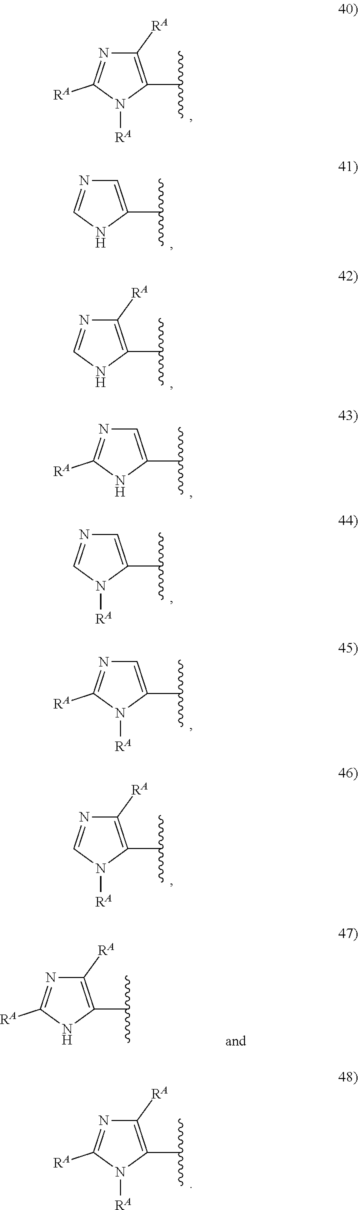

Y¹ is a bond, Y² is a bond, m is 4, R¹ and R² join together to form C₃cycloalkyl and R⁴ is H.

In an embodiment,

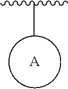

is selected from the group consisting of:

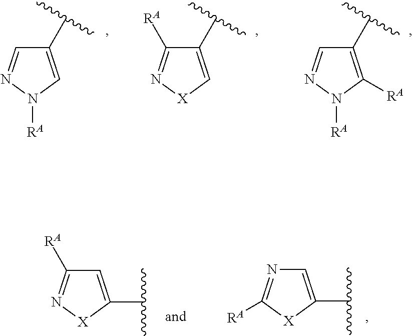

and X is selected from O, NH and S.

In an embodiment,

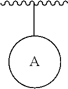

is selected from the group consisting of:

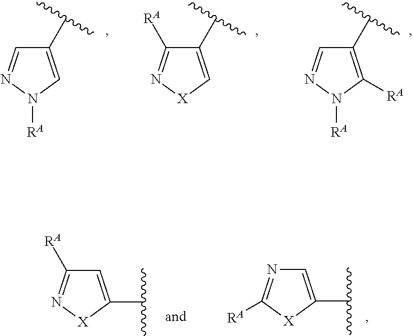

and X is selected from O, NH and S, Y¹ is a bond, Y² is a bond, m is 4, R¹ and R² join together to form C₃cycloalkyl and R⁴ is H.

In an embodiment,

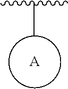

is selected from the group consisting of:

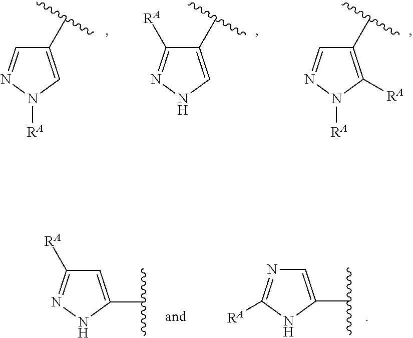

In an embodiment,

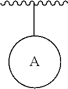

is selected from the group consisting of:

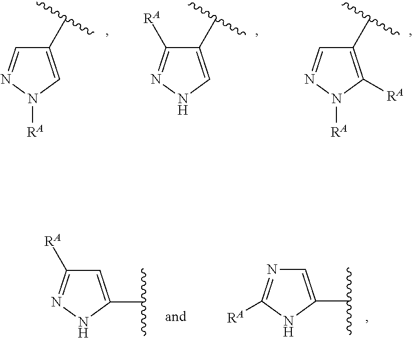

Y¹ is a bond, Y² is a bond, m is 4, R¹ and R² join together to form C₃cycloalkyl and R⁴ is H.

In an embodiment, i)

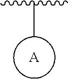

is selected from the group consisting of:

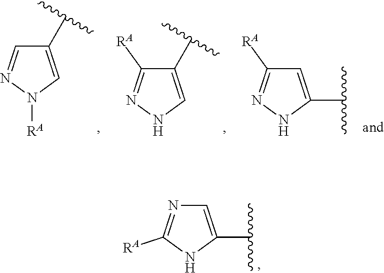

(e.g.

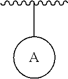

is

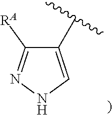

and R^{A} is selected from
a) halo,
b) cyano,
c) C₁-C₆alkyl,
d) C₁-C₆haloalkyl,
e) C₁-C₆hydroxyalkyl,
f) O—C₁-C₆alkyl,
g) C(═O)—O—C₁-C₆alkyl,
h) C₁-C₆alkylene-O—C₁-C₆alkyl,
i) O—C₁-C₆alkylene-O—C₁-C₆alkyl,
j) C₃-C₆cycloalkyl substituted by 0-3 R³ groups,
k) 5-10 membered heteroaryl comprising 1-4 heteroatoms independently selected from N, O and S substituted by 0-3 R³ groups,
l) 5-10 membered partially saturated heterocyclyl comprising 1-4 heteroatoms independently selected from N, O and S substituted by 0-3 R³ groups,
m) O—C₃-C₆cycloalkyl substituted by 0-3 R³ groups,
n) C≡C-5-10 membered heteroaryl comprising 1-4 heteroatoms independently selected from N, O and S substituted by 0-3 R³ groups,
o) S-5-10 membered heteroaryl comprising 1-4 heteroatoms independently selected from N, O and S substituted by 0-3 R³ groups,
p) C₁-C₆alkylene-5-10 membered heteroaryl comprising 1-4 heteroatoms independently selected from N, O and S substituted by 0-3 R³ groups,
q) O—C₁-C₆haloalkyl,
r) O—C₁-C₆alkylene-N(C₁-C₆alkyl)₂,
s) O—C₁-C₆hydroxyalkylene-O—C₁-C₆alkyl,
t) O—C₁-C₆alkylene-C₃-C₆cycloalkyl substituted by 0-3 R³ groups,
u) O—C₁-C₆alkylene-3-6 membered heterocyclyl comprising 1 or 2 heteroatoms independently selected from O, N and S substituted by 0-3 R³ groups,
v) O—C₁-C₆alkylene-C(═O)—N(C₁-C₆alkyl)₂,
w) C₁-C₆alkylene-5-10 membered partially saturated heterocyclyl comprising 1-4 heteroatoms independently selected from N, O and S substituted by 0-3 R³ groups,
x) O—C₁-C₆alkylene-7-10 membered spiroheterocyclyl comprising 1 or 2 heteroatoms independently selected from O, N and S substituted by 0-3 R³ groups,
y) O—C₁-C₆alkylene-S(O)₂—C₁-C₆alkyl,
z) O—C₁-C₆hydroxyalkyl,
aa) O—C₁-C₆alkylene-5-10 membered heteroaryl comprising 1-4 heteroatoms independently selected from N, O and S substituted by 0-3 R³ groups,
bb) O-5-10 membered heteroaryl comprising 1-4 heteroatoms independently selected from N, O and S substituted by 0-3 R³ groups,
cc) O-3-6 membered heterocyclyl comprising 1 or 2 heteroatoms independently selected from O, N and S substituted by 0-3 R³ groups,
dd) C≡C—C₃-C₆cycloalkyl substituted by 0-3 R³ groups,
ee) S—C₁-C₆haloalkyl,
ff) O—C₁-C₆alkylene-O-3-6 membered heterocyclyl comprising 1 or 2 heteroatoms independently selected from O, N and S substituted by 0-3 R³ groups
gg) and 3 to 6 membered heterocyclyl comprising 1 or 2 heteroatoms selected from N, O and S substituted by 0-3 R³ groups; or
ii)

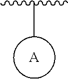

is

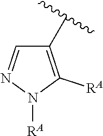

and the R^{A} substituents join together to form, with the ring atoms to which they are attached, a 4 to 6 membered heterocyclyl comprising 1 to 3 heteroatoms independently selected from N, O and S.

In an embodiment, i)

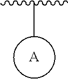

is selected from the group consisting of:

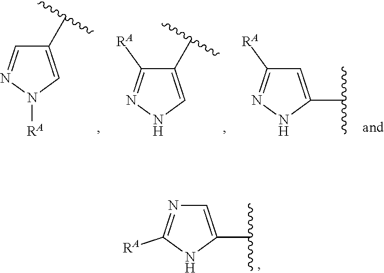

(e.g.

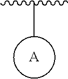

is

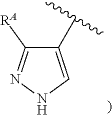

and R^{A} is selected from
a) halo,
b) cyano,
c) C₁-C₆alkyl,
d) C₁-C₆haloalkyl,
e) C₁-C₆hydroxyalkyl,
f) O—C₁-C₆alkyl,
g) C(═O)—O—C₁-C₆alkyl,
h) C₁-C₆alkylene-O—C₁-C₆alkyl,
i) O—C₁-C₆alkylene-O—C₁-C₆alkyl,
j) C₃-C₆cycloalkyl substituted by 0-3 R³ groups,
k) 5-10 membered heteroaryl comprising 1-4 heteroatoms independently selected from N, O and S substituted by 0-3 R³ groups,
l) 5-10 membered partially saturated heterocyclyl comprising 1-4 heteroatoms independently selected from N, O and S substituted by 0-3 R³ groups,
m) O—C₃-C₆cycloalkyl substituted by 0-3 R³ groups,
n) C≡C-5-10 membered heteroaryl comprising 1-4 heteroatoms independently selected from N, O and S substituted by 0-3 R³ groups,
o) S-5-10 membered heteroaryl comprising 1-4 heteroatoms independently selected from N, O and S substituted by 0-3 R³ groups,
p) C₁-C₆alkylene-5-10 membered heteroaryl comprising 1-4 heteroatoms independently selected from N, O and S substituted by 0-3 R³ groups,
q) O—C₁-C₆haloalkyl,
r) O—C₁-C₆alkylene-N(C₁-C₆alkyl)₂,
s) O—C₁-C₆hydroxyalkylene-O—C₁-C₆alkyl,
t) O—C₁-C₆alkylene-C₃-C₆cycloalkyl substituted by 0-3 R³ groups,
u) O—C₁-C₆alkylene-3-6 membered heterocyclyl comprising 1 or 2 heteroatoms independently selected from O, N and S substituted by 0-3 R³ groups,
v) O—C₁-C₆alkylene-C(═O)—N(C₁-C₆alkyl)₂,
w) C₁-C₆alkylene-5-10 membered partially saturated heterocyclyl comprising 1-4 heteroatoms independently selected from N, O and S substituted by 0-3 R³ groups,
x) O—C₁-C₆alkylene-7-10 membered spiroheterocyclyl comprising 1 or 2 heteroatoms independently selected from O, N and S substituted by 0-3 R³ groups,
y) O—C₁-C₆alkylene-S(O)₂—C₁-C₆alkyl,
z) O—C₁-C₆hydroxyalkyl,
aa) O—C₁-C₆alkylene-5-10 membered heteroaryl comprising 1-4 heteroatoms independently selected from N, O and S substituted by 0-3 R³ groups,
bb) O-5-10 membered heteroaryl comprising 1-4 heteroatoms independently selected from N, O and S substituted by 0-3 R³ groups,
cc) O-3-6 membered heterocyclyl comprising 1 or 2 heteroatoms independently selected from O, N and S substituted by 0-3 R³ groups,
dd) C≡C—C₃-C₆cycloalkyl substituted by 0-3 R³ groups,
ee) S—C₁-C₆haloalkyl,
ff) O—C₁-C₆alkylene-O-3-6 membered heterocyclyl comprising 1 or 2 heteroatoms independently selected from O, N and S substituted by 0-3 R³ groups
gg) and 3 to 6 membered heterocyclyl comprising 1 or 2 heteroatoms selected from N, O and S substituted by 0-3 R³ groups; or
ii)

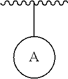

is

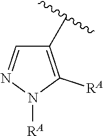

and the R^{A} substituents join together to form with the ring atoms to which they are attached, a 4 to 6 membered heterocyclyl comprising 1 to 3 heteroatoms independently selected from N, O and S, Y¹ is a bond, Y² is a bond, m is 4, R¹ and R² join together to form C₃cycloalkyl and R⁴ is H.

In an embodiment, each R³ is independently selected from the group consisting of halo (e.g. fluoro), C₁-C₆alkyl (e.g. methyl), hydroxyl, cyano, S(O₂)—C₁-C₆alkyl (e.g. S(O₂)CH₃), C(═O)—C₁-C₆alkyl (e.g. C(═O)CH₃), O—C₁-C₆alkyl (e.g. OCH₃) and C₁-C₆haloalkyl (e.g. C₁haloalkyl, e.g. CHF₂), or wherein two R³ substituents on the same ring atom join together to form ═O.

In an embodiment,

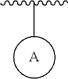

is selected from the group consisting of:

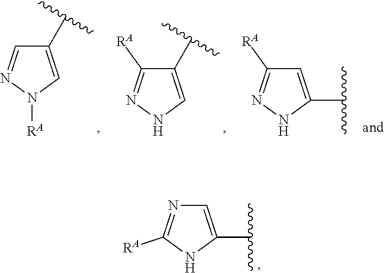

and R^{A} is selected from halo, cyano, C₁-C₆alkyl, C₁-C₆haloalkyl, C₁-C₆hydroxyalkyl, O—C₁-C₆alkyl, C(═O)—O—C₁-C₆alkyl, C₁-C₆alkylene-O—C₁-C₆alkyl, O—C₁-C₆alkylene-O—C₁-C₆alkyl, C₃-C₆cycloalkyl and 3 to 6 membered heterocyclyl comprising 1 or 2 heteroatoms selected from N, O and S; or
ii)

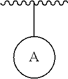

is

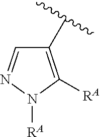

and the R^{A} substituents join together to form, with the ring atoms to which they are attached, a 4 to 6 membered heterocyclyl comprising 1 to 3 heteroatoms independently selected from N, O and S.

In an embodiment,

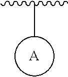

is selected from the group consisting of:

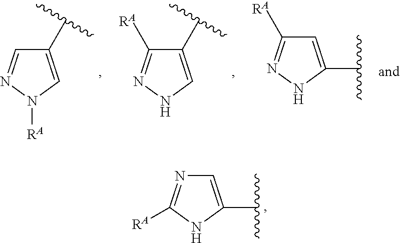

and R^{A} is selected from halo, cyano, C₁-C₆alkyl, C₁-C₆haloalkyl, C₁-C₆hydroxyalkyl, O—C₁-C₆alkyl, C(═O)—O—C₁-C₆alkyl, C₁-C₆alkylene-O—C₁-C₆alkyl, O—C₁-C₆alkylene-O—C₁-C₆alkyl, C₃-C₆cycloalkyl and 3 to 6 membered heterocyclyl comprising 1 or 2 heteroatoms selected from N, O and S; or
ii)

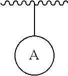

is

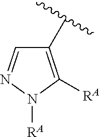

and the R^{A} substituents join together to form, with the ring atoms to which they are attached, a 4 to 6 membered heterocyclyl comprising 1 to 3 heteroatoms independently selected from N, O and S, Y¹ is a bond, Y² is a bond, m is 4, R¹ and R² join together to form C₃cycloalkyl and R⁴ is H.

In an embodiment,

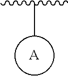

is

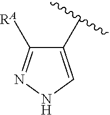

In an embodiment,

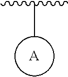

is

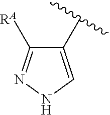

Y¹ is a bond, Y² is a bond, m is 4, R¹ and R² join together to form C₃cycloalkyl and R⁴ is H.

In an embodiment,

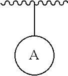

is

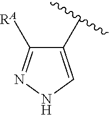

and R^{A} is CH₃ or OCH₃.

In an embodiment,

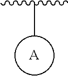

is

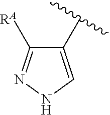

and R^{A} is CH₃, OCH₃ or OCH₂CH₃.

In an embodiment,

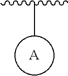

is

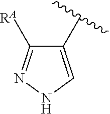

and R^{A} is CH₃ or OCH₃, Y¹ is a bond, Y² is a bond, m is 4, R¹ and R² join together to form C₃cycloalkyl and R⁴ is H.

In an embodiment,

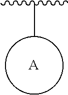

is

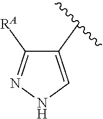

and R^{A} is CH₃, OCH₃ or OCH₂CH₃, Y¹ is a bond, Y² is a bond, m is 4, R¹ and R² join together to form C₃cycloalkyl and R⁴ is H.

In an embodiment, each L¹ is independently selected from bond, O, C(═O), *C(═O)—O**, C₁-C₆alkylene, C₁-C₆haloalkylene, *O—C₁-C₆alkylene**, C₁-C₆alkylene-O—C₁-C₆alkylene, C₁-C₆hydroxyalkylene, C₃-C₆cycloalkylene, 3-6 membered heterocyclylene (e.g. comprising 1 heteroatom which is O) and O—C₁-C₆alkylene-O, wherein * indicates the point of attachment to

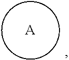

and ** indicates the point of attachment to X¹;
and each X¹ is independently selected from H, halo, cyano, hydroxyl, C₁-C₆alkyl, C₁-C₆haloalkyl, C₃-C₆cycloalkyl, O—C₁-C₆alkyl, C₁-C₆hydroxyalkyl and 3-6 membered heterocyclyl (e.g. comprising 1 heteroatom that is O).

In an embodiment, the compound of formula (I) is a compound of formula (Id-I):

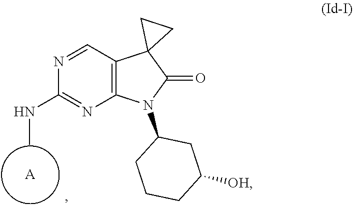

wherein

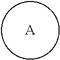

is as described in any one of the embodiments, and each L¹ is independently selected from bond, O, C(═O), *C(═O)—O**, C₁-C₆alkylene, C₁-C₆haloalkylene, *O—C₁-C₆alkylene**, C₁-C₆alkylene-O—C₁-C₆alkylene, C₁-C₆hydroxyalkylene, C₃-C₆cycloalkylene, 3-6 membered heterocyclylene (e.g. comprising 1 heteroatom which is O) and O—C₁-C₆alkylene-O, wherein * indicates the point of attachment to

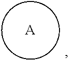

and ** indicates the point of attachment to X¹;
and each X¹ is independently selected from H, halo, cyano, hydroxyl, C₁-C₆alkyl, C₁-C₆haloalkyl, C₃-C₆cycloalkyl, O—C₁-C₆alkyl, C₁-C₆hydroxyalkyl and 3-6 membered heterocyclyl (e.g. comprising 1 heteroatom that is O).

In an embodiment, each L¹ is independently selected from bond, O, C(═O), *C(═O)—O**, C₁-C₂alkylene, C₁haloalkylene, *O—C₁-C₂alkylene**, C₁-C₂alkylene-O—C₁alkylene, C₁-hydroxyalkylene, C₃cycloalkylene, 5-6 membered heterocyclylene comprising 1 heteroatom which is O and O—C₂alkylene-O, wherein * indicates the point of attachment to

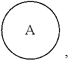

and ** indicates the point of attachment to X¹.

In an embodiment, the compound of formula (I) is a compound of formula (Id-I):

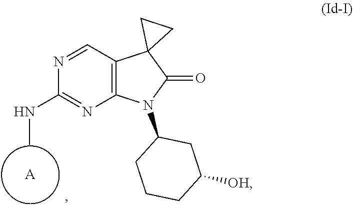

wherein

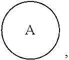

is as described in any one of the embodiments, and each L¹ is independently selected from bond, O, C(═O), *C(═O)—O**, C₁-C₂alkylene, C₁haloalkylene, *O—C₁-C₂alkylene**, C₁-C₂alkylene-O—C₁alkylene, C₁-hydroxyalkylene, C₃cycloalkylene, 5-6 membered heterocyclylene comprising 1 heteroatom which is O and O—C₂alkylene-O, wherein * indicates the point of attachment to

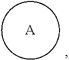

and ** indicates the point of attachment to X¹.

In an embodiment, each X¹ is independently selected from H, halo, cyano, hydroxyl, C₁-C₂alkyl (e.g. C₁alkyl), C₁haloalkyl, C₃cycloalkyl, O—C₁-C₂alkyl, C₁hydroxyalkyl and 5-6 membered heterocyclyl comprising 1 heteroatom that is O.

In an embodiment, the compound of formula (I) is a compound of formula (Id-I):

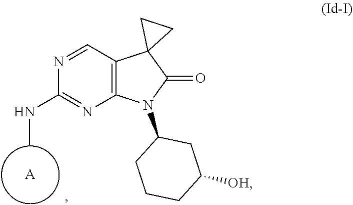

wherein

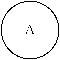

is as described in any one of the embodiments, and each X¹ is independently selected from H, halo, cyano, hydroxyl, C₁-C₂alkyl (e.g. C₁alkyl), C₁haloalkyl, C₃cycloalkyl, O—C₁-C₂alkyl, C₁hydroxyalkyl and 5-6 membered heterocyclyl comprising 1 heteroatom that is O.

In an embodiment, the compound of formula (I) is a compound of formula (Id-I):

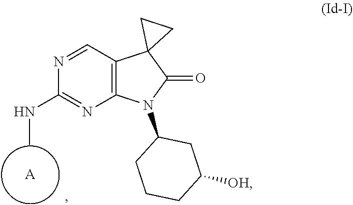

wherein

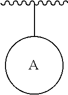

is selected from the group consisting of

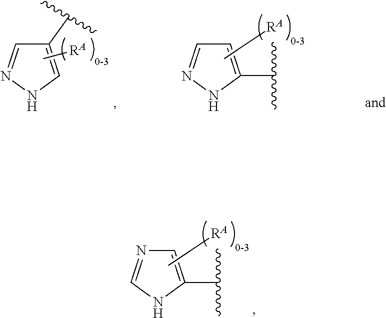

and R^{A} is selected from the list consisting of C₁-C₆alkyl, C₁-C₆alkylene-O—C₁-C₆alkyl, C₁-C₆haloalkyl, C(═O)—O—C₁-C₆alkyl, C₁-C₆hydroxyalkyl, 3-6 membered heteroatom comprising 1 heteroatom that is O, halo, O—C₁-C₆alkyl, C₃-C₆cycloalkyl, cyano and O—C₁-C₆alkylene-O—C₁-C₆alkyl.

In an embodiment, the compound of formula (I) is a compound of formula (Id-I):

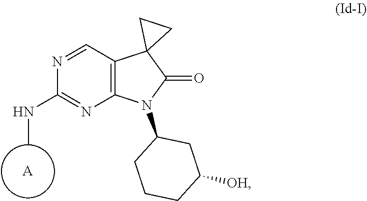

wherein

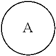

is as described in any one of the embodiments, and R^{A} is selected from the list consisting of C₆-C₆alkyl, C₁-C₆alkylene-O—C₁-C₆alkyl, C₁-C₆haloalkyl, C(═O)—O—C₁-C₆alkyl, C₁-C₆hydroxyalkyl, 3-6 membered heteroatom comprising 1 heteroatom that is O, halo, O—C₁-C₆alkyl, C₃-C₆cycloalkyl, cyano and O—C₁-C₆alkylene-O—C₁-C₆alkyl.

In an embodiment, the compound of formula (I) is a compound of formula (Id-I):

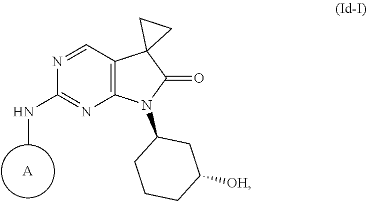

wherein

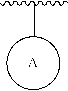

is selected from the group consisting of

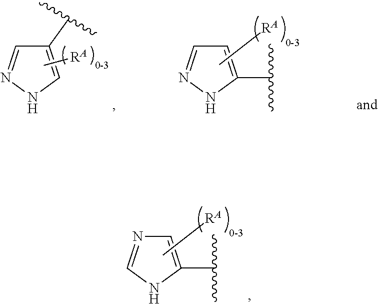

and R^{A} is selected from the list consisting of OH₃, CH₂CH₂OCH₃, CHF₂, C(═O)—O—CH₃, CH₂OH, 5 membered heteroatom comprising 1 heteroatom that is O, 6 membered heteroatom comprising 1 heteroatom that is O, CH₂OCH₃, 5° C. H₃, Cl, C₃cycloalkyl, OF₃, cyano, OCH₂CH₂OCH₃ and OCH₂CH₃.

In an embodiment, the compound of formula (I) is a compound of formula (Id-I):

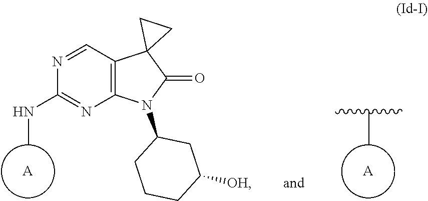

is

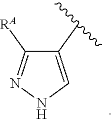

In an embodiment, the compound of formula (I) is a compound of formula (Id-I):

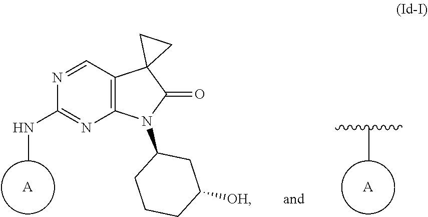

is

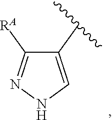

and R^{A} is selected from the list consisting of CH₃ and OCH₃.

In an embodiment, the compound of formula (I) is a compound of formula (Id-I):

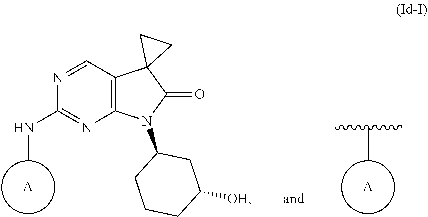

is

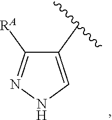

and R^{A} is selected from the list consisting of CH₃, OCH₃ and OCH₂CH₃.

In an embodiment, R¹ and R² join together to form C₃-C₄cycloalkyl or C₃-C₄cyclohaloalkyl, and

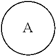

is a 5 membered heteroaryl selected from the group consisting of

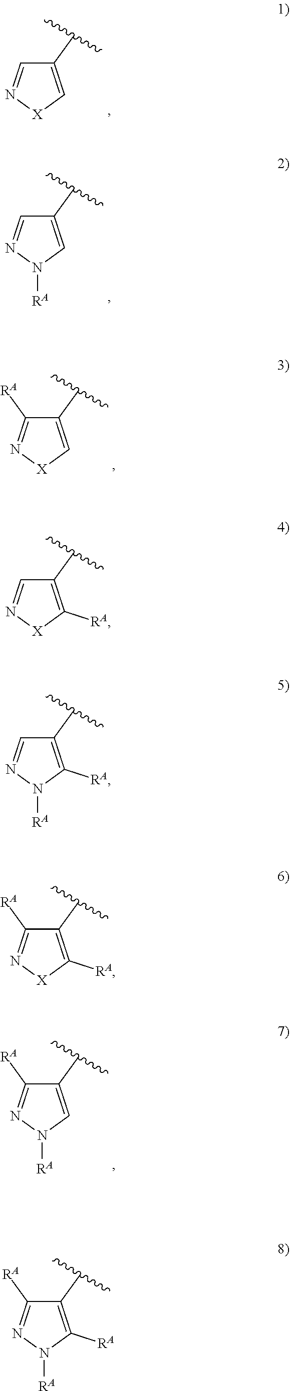

wherein X is selected from O, NH and S (e.g. X is NH).

In an embodiment, R¹ and R² join together to form C₃-C₄cycloalkyl, and

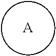

is a 5 membered heteroaryl selected from the group consisting of

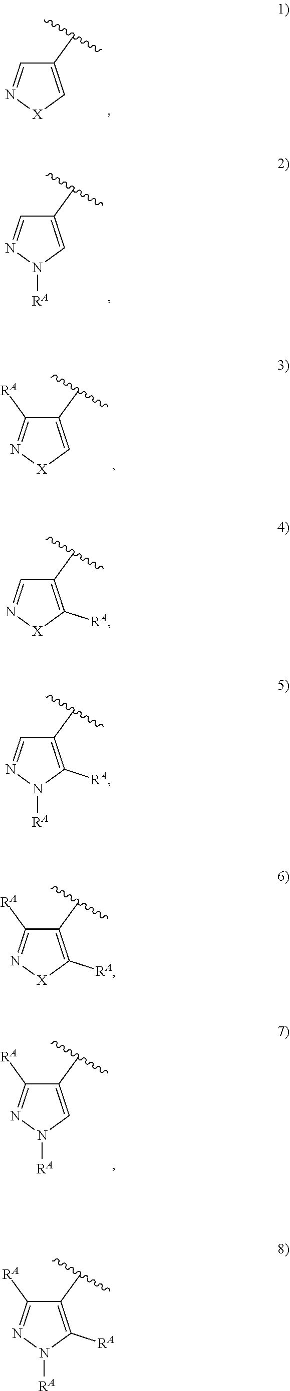

wherein X is selected from O, NH and S (e.g. X is NH).

In an embodiment, R¹ and R² join together to form C₃cycloalkyl, and

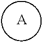

is a 5 membered heteroaryl selected from the group consisting of

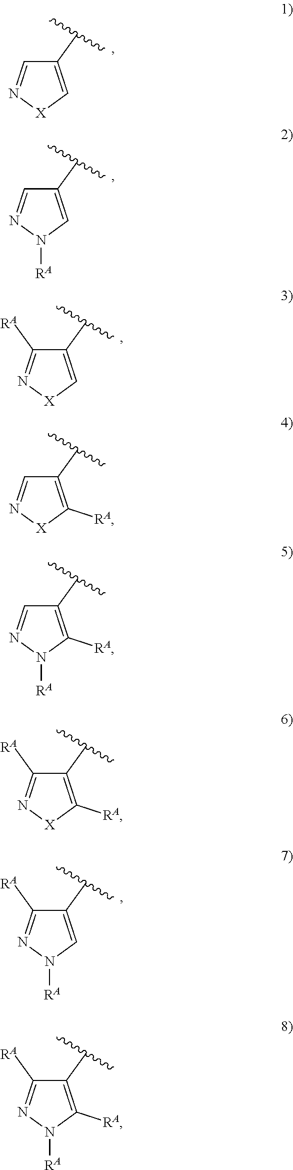

wherein X is selected from O, NH and S (e.g. X is NH).

In an embodiment, R⁴ is H, and

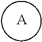

is a 5 membered heteroaryl selected from the group consisting of

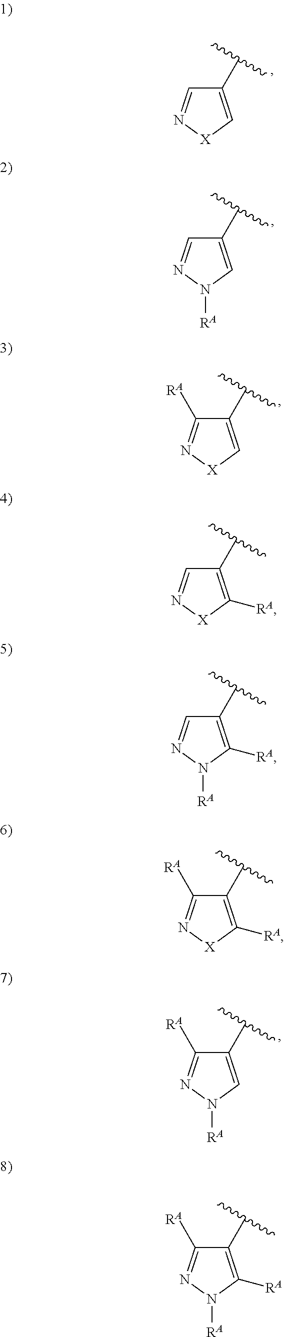

wherein X is selected from O, NH and S (e.g. X is NH).

In an embodiment, R¹ and R² join together to form C₃-C₄cycloalkyl, and R⁴ is H, and

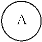

is a 5 membered heteroaryl selected from the group consisting of

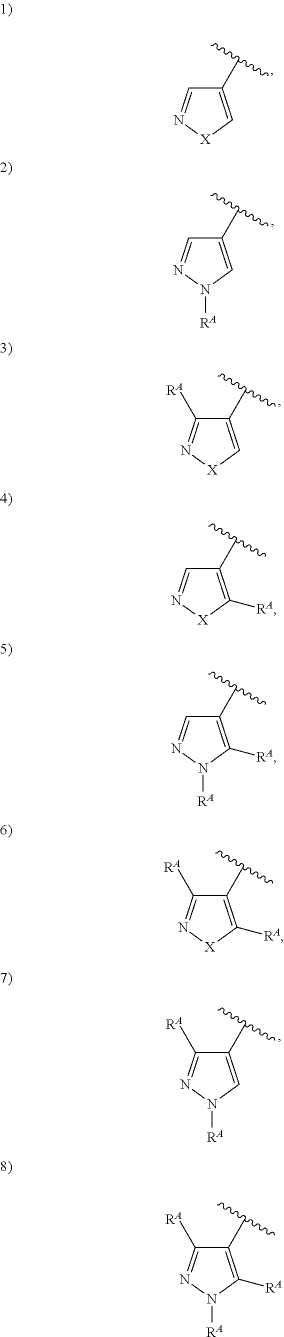

wherein X is selected from O, NH and S (e.g. X is NH).

In an embodiment, R¹ and R² join together to form C₃cycloalkyl, and R⁴ is H, and

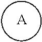

is a 5 membered heteroaryl selected from the group consisting of

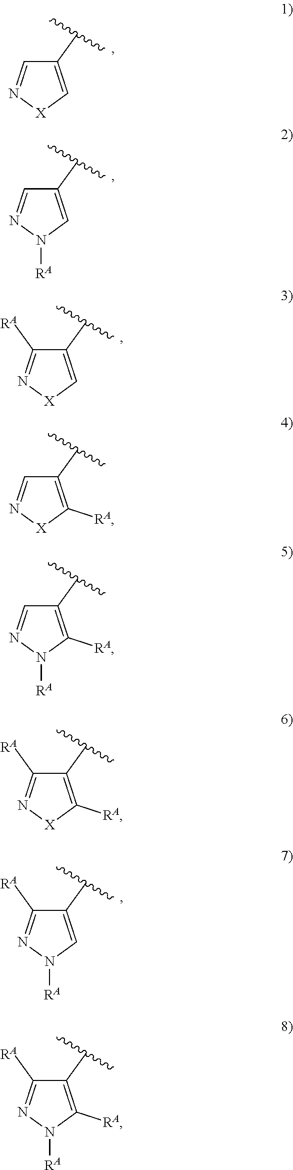

wherein X is selected from O, NH and S (e.g. X is NH).

In an embodiment, m is 4 and R¹ and R² join together to form C₃-C₄cycloalkyl, and

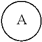

is a 5 membered heteroaryl selected from the group consisting of

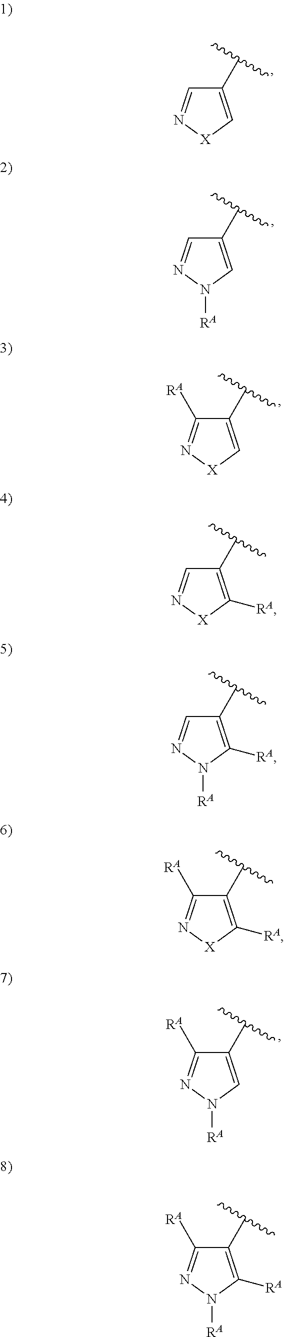

wherein X is selected from O, NH and S (e.g. X is NH).

In an embodiment, m is 4 and R¹ and R² join together to form C₃cycloalkyl, and

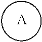

is a 5 membered heteroaryl selected from the group consisting of

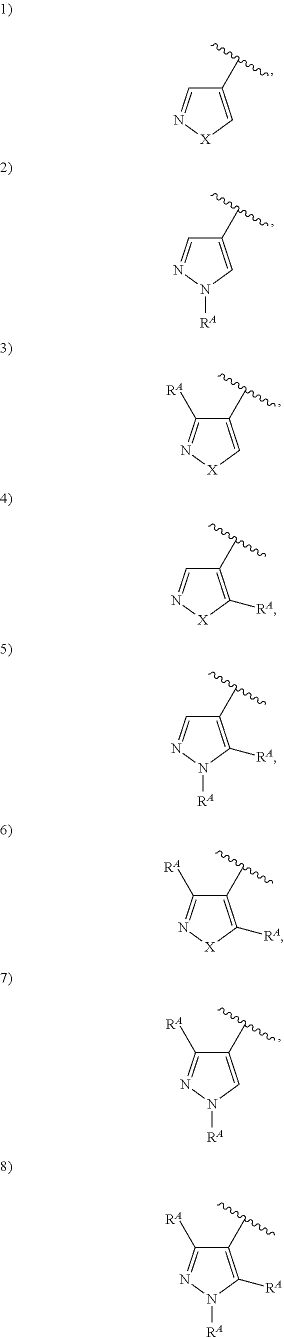

wherein X is selected from O, NH and S (e.g. X is NH).

In an embodiment, m is 4 and R⁴ is H, and

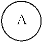

is a 5 membered heteroaryl selected from the group consisting of

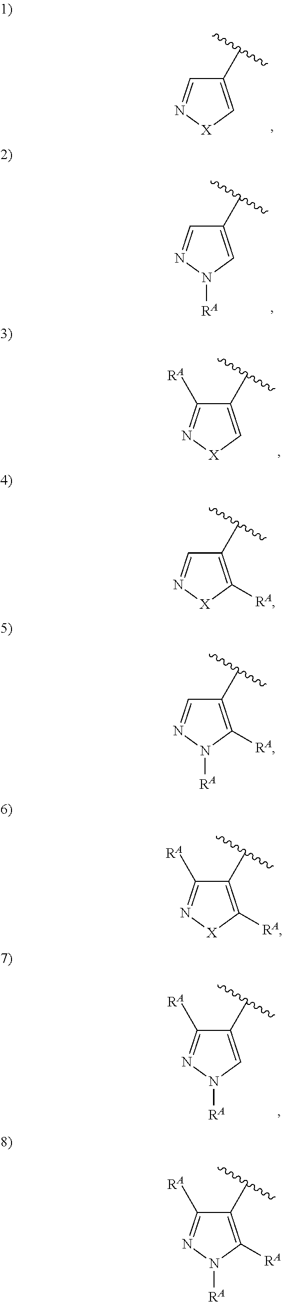

wherein X is selected from O, NH and S (e.g. X is NH).

In an embodiment, m is 4, R¹ and R² join together to form C₃-C₄cycloalkyl and R⁴ is H, and

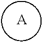

is a 5 membered heteroaryl selected from the group consisting of

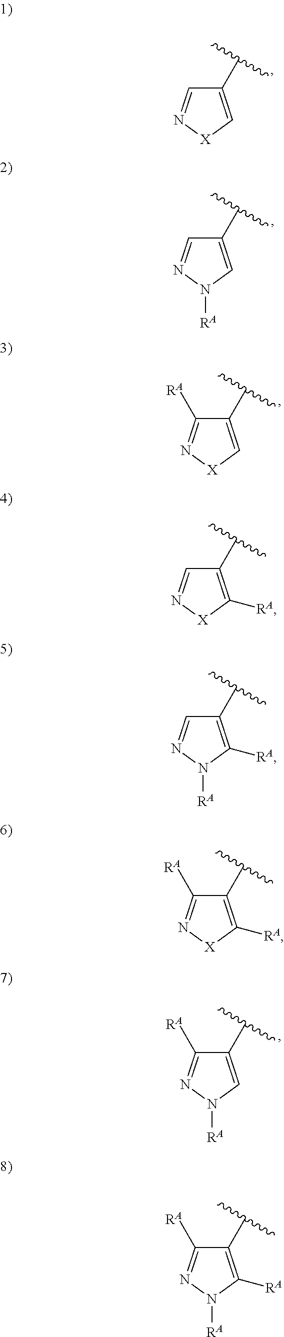

wherein X is selected from O, NH and S (e.g. X is NH).

In an embodiment, m is 4, R¹ and R² join together to form C₃cycloalkyl and R⁴ is H, and

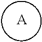

is a 5 membered heteroaryl selected from the group consisting of

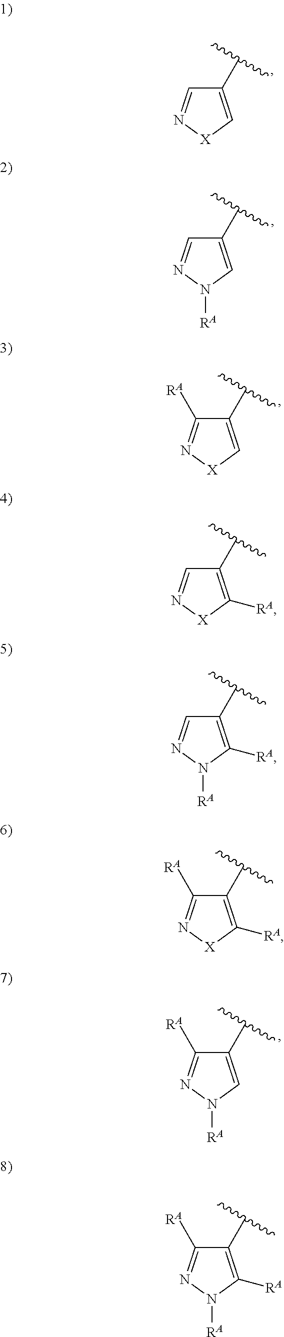

wherein X is selected from O, NH and S (e.g. X is NH).

In an embodiment, Y¹ is a bond, R¹ and R² join together to form C₃-C₄cycloalkyl, and R⁴ is H, and

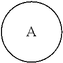

is a 5 membered heteroaryl selected from the group consisting of

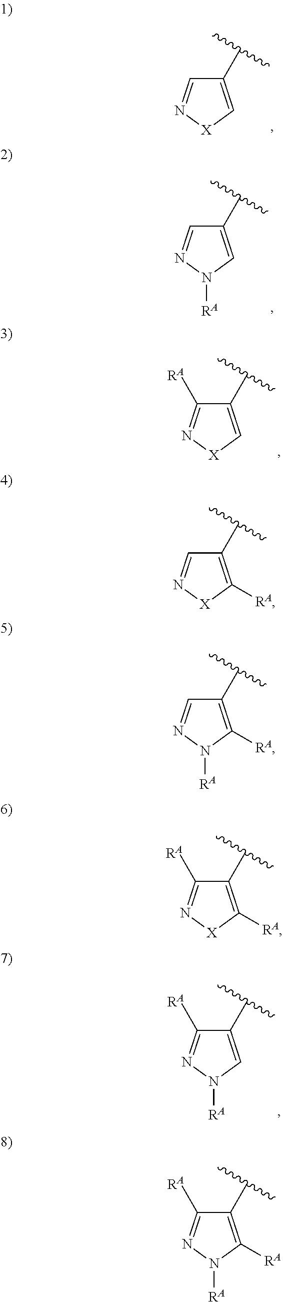

wherein X is selected from O, NH and S (e.g. X is NH).

In an embodiment, Y¹ is a bond, R¹ and R² join together to form C₃cycloalkyl, and R⁴ is H, and

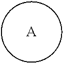

is a 5 membered heteroaryl selected from the group consisting of

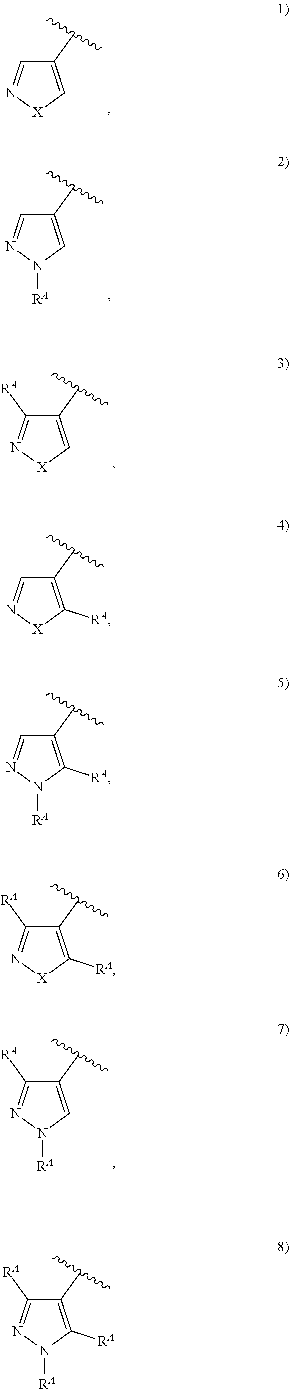

wherein X is selected from O, NH and S (e.g. X is NH).

In an embodiment, Y¹ is a bond, m is 4 and R¹ and R² join together to form C₃-C₄cycloalkyl, and

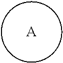

is a 5 membered heteroaryl selected from the group consisting of

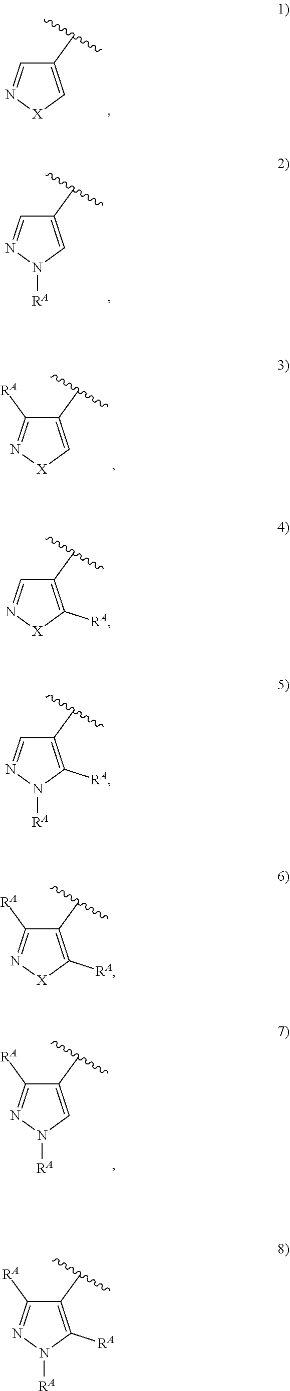

wherein X is selected from O, NH and S (e.g. X is NH).

In an embodiment, Y¹ is a bond, m is 4 and R¹ and R² join together to form C₃cycloalkyl, and

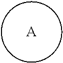

is a 5 membered heteroaryl selected from the group consisting of

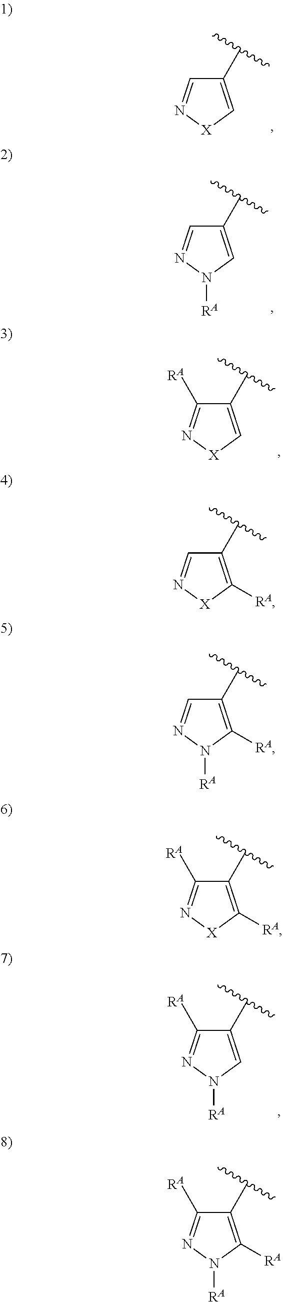

wherein X is selected from O, NH and S (e.g. X is NH).

In an embodiment, Y¹ is a bond, m is 4 and R⁴ is H, and

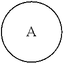

is a 5 membered heteroaryl selected from the group consisting of

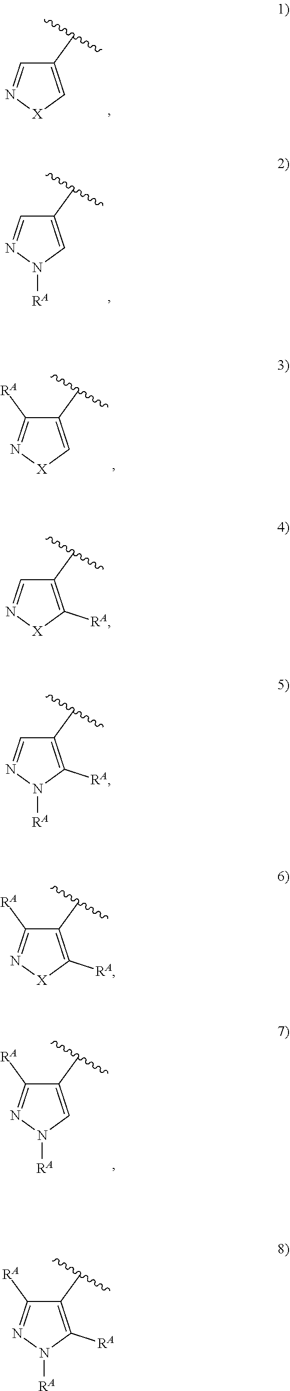

wherein X is selected from O, NH and S (e.g. X is NH).

In an embodiment, Y¹ is a bond, m is 4, R¹ and R² join together to form C₃-C₄cycloalkyl and R⁴ is H, and

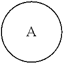

is a 5 membered heteroaryl selected from the group consisting of

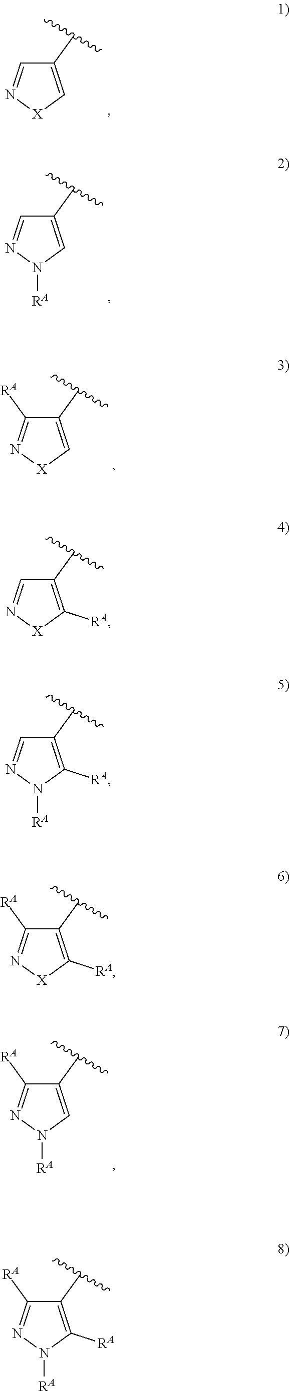

wherein X is selected from O, NH and S (e.g. X is NH).

In an embodiment, Y¹ is a bond, m is 4, R¹ and R² join together to form C₃cycloalkyl and R⁴ is H, and

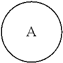

is a 5 membered heteroaryl selected from the group consisting of

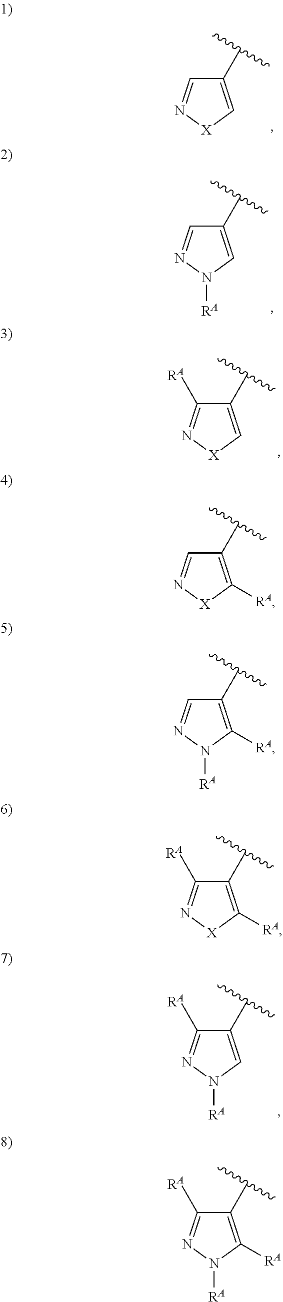

wherein X is selected from O, NH and S (e.g. X is NH).

In an embodiment, Y² is a bond, R¹ and R² join together to form C₃-C₄cycloalkyl, and R⁴ is H, and

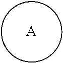

is a 5 membered heteroaryl selected from the group consisting of

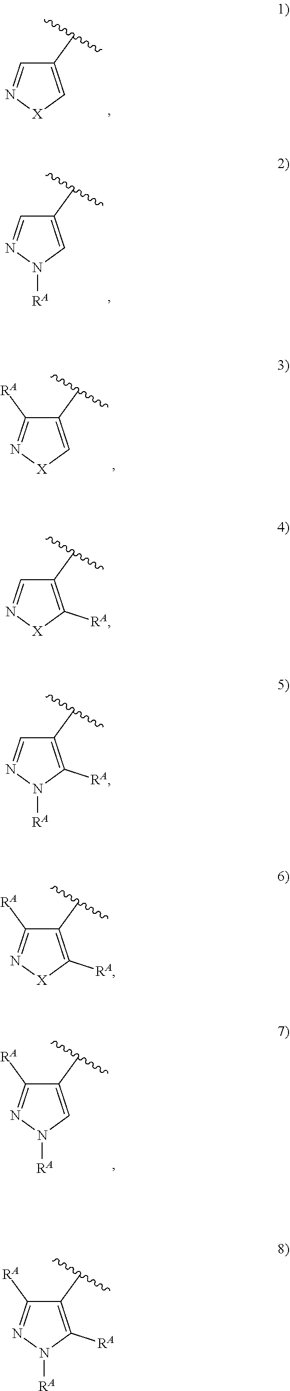

wherein X is selected from O, NH and S (e.g. X is NH).

In an embodiment, Y² is a bond, R¹ and R² join together to form C₃cycloalkyl, and R⁴ is H, and

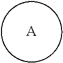

is a 5 membered heteroaryl selected from the group consisting of

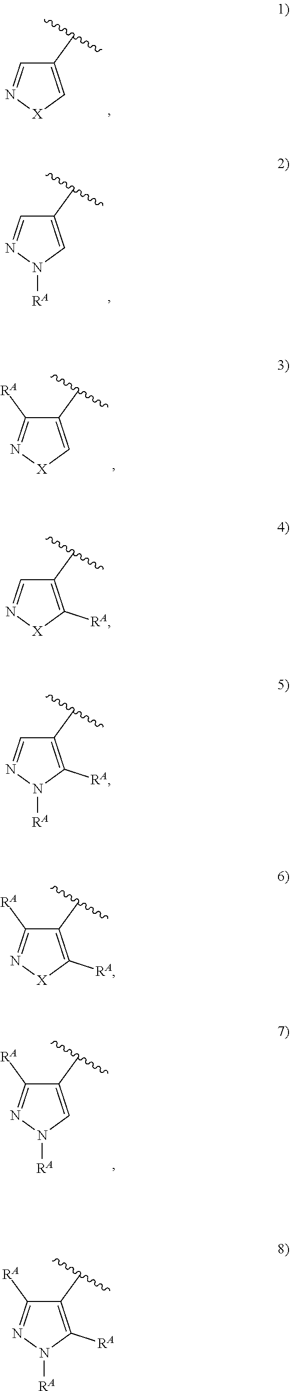

wherein X is selected from O, NH and S (e.g. X is NH).

In an embodiment, Y² is a bond, m is 4 and R¹ and R² join together to form C₃-C₄cycloalkyl, and

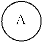

is a 5 membered heteroaryl selected from the group consisting of

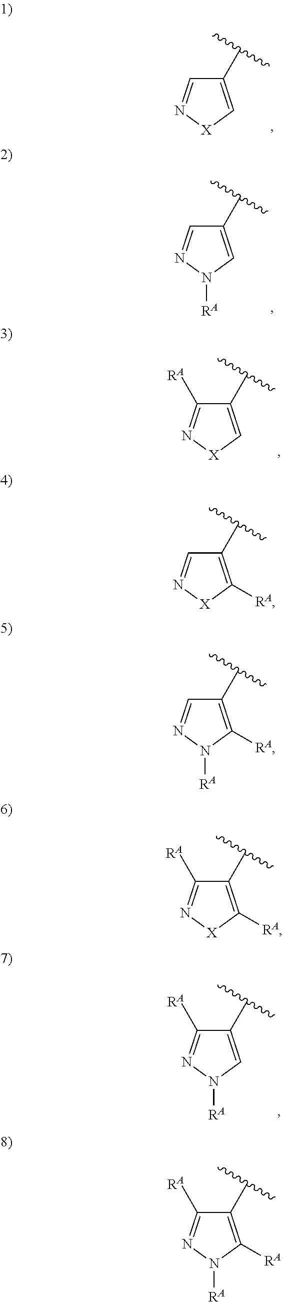

wherein X is selected from O, NH and S (e.g. X is NH).

In an embodiment, Y² is a bond, m is 4 and R¹ and R² join together to form C₃cycloalkyl, and

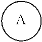

is a 5 membered heteroaryl selected from the group consisting of

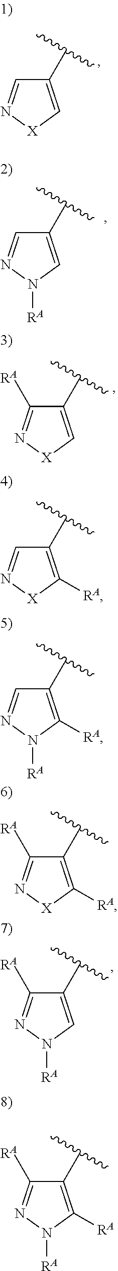

wherein X is selected from O, NH and S (e.g. X is NH).

In an embodiment, Y² is a bond, m is 4 and R⁴ is H, and

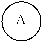

is a 5 membered heteroaryl selected from the group consisting of

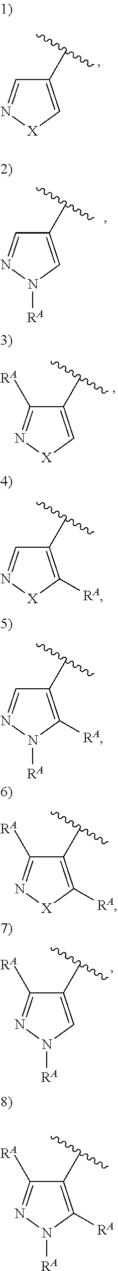

wherein X is selected from O, NH and S (e.g. X is NH).

In an embodiment, Y² is a bond, m is 4, R¹ and R² join together to form C₃-C₄cycloalkyl and R⁴ is H, and

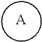

is a 5 membered heteroaryl selected from the group consisting of

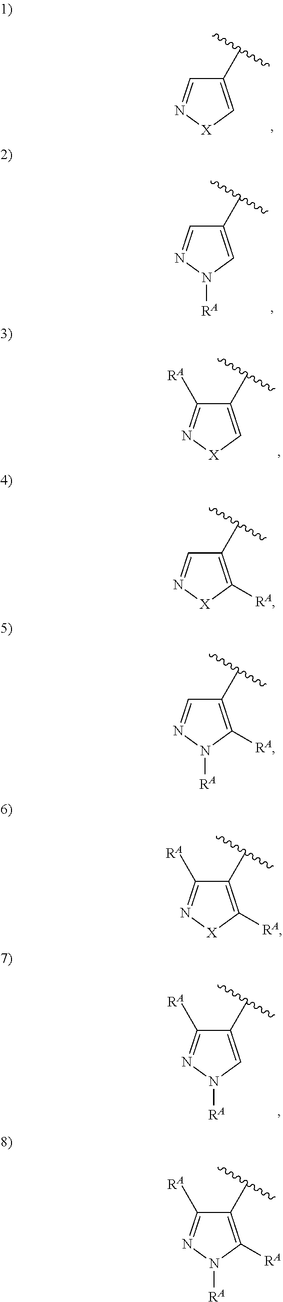

wherein X is selected from O, NH and S (e.g. X is NH).

In an embodiment, Y² is a bond, m is 4, R¹ and R² join together to form C₃cycloalkyl and R⁴ is and

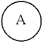

is a 5 membered heteroaryl selected from the group consisting of

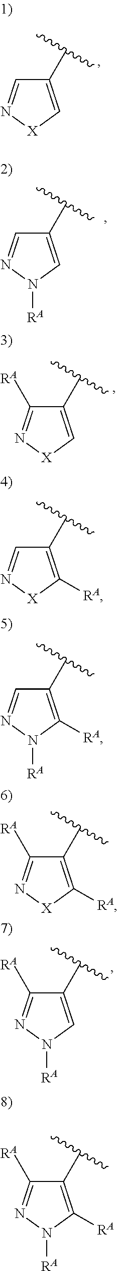

wherein X is selected from O, NH and S (e.g. X is NH).

In an embodiment, Y¹ is a bond, Y² is a bond, R¹ and R² join together to form C₃-C₄cycloalkyl, and R⁴ is H, and

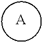

is a 5 membered heteroaryl selected from the group consisting of

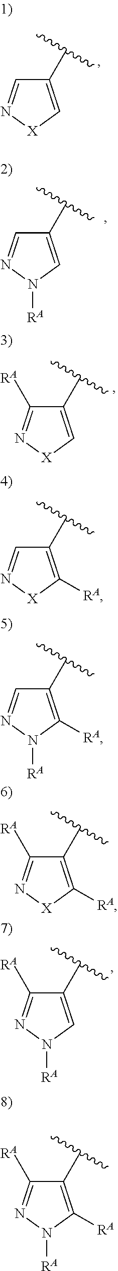

wherein X is selected from O, NH and S (e.g. X is NH).

In an embodiment, Y¹ is a bond, Y² is a bond, R¹ and R² join together to form C₃cycloalkyl, and R⁴ is H, and

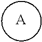

is a 5 membered heteroaryl selected from the group consisting of

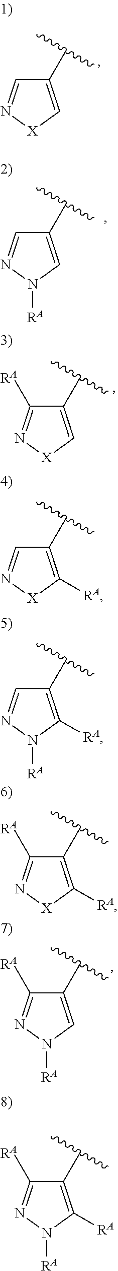

wherein X is selected from O, NH and S (e.g. X is NH).

In an embodiment, Y¹ is a bond, Y² is a bond, m is 4 and R¹ and R² join together to form C₃-C₄cycloalkyl, and

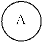

is a 5 membered heteroaryl selected from the group consisting of

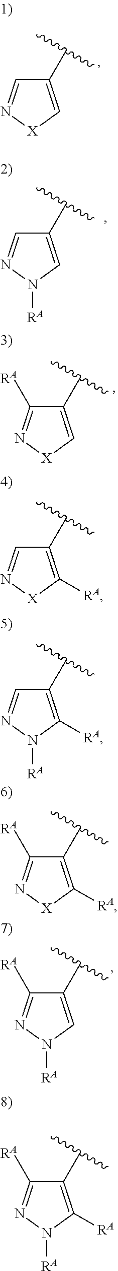

wherein X is selected from O, NH and S (e.g. X is NH).

In an embodiment, Y¹ is a bond, Y² is a bond, m is 4 and R¹ and R² join together to form C₃cycloalkyl, and

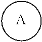

is a 5 membered heteroaryl selected from the group consisting of 1)

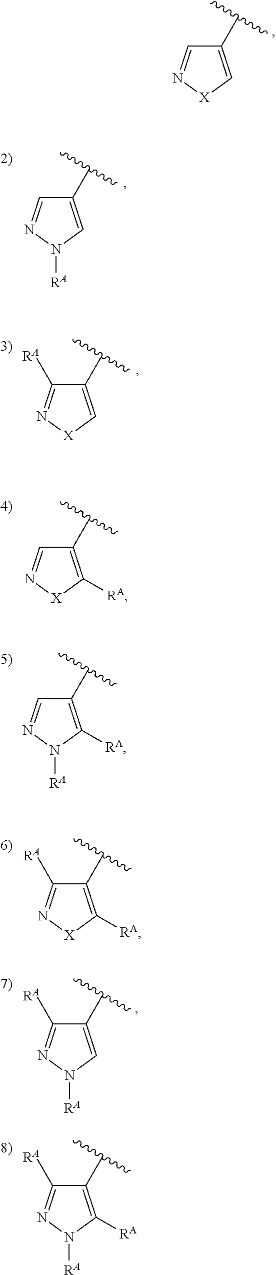

wherein X is selected from O, NH and S (e.g. X is NH).

In an embodiment, Y¹ is a bond, Y² is a bond, m is 4 and R⁴ is H, and

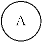

is a 5 membered heteroaryl selected from the group consisting of

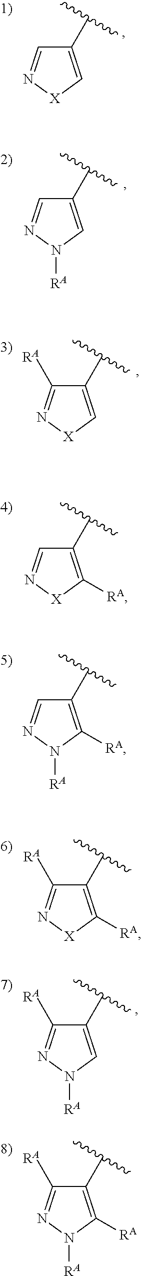

wherein X is selected from O, NH and S (e.g. X is NH).

In an embodiment, Y¹ is a bond, Y² is a bond, m is 4, R¹ and R² join together to form C₃-C₄cycloalkyl and R⁴ is H, and

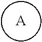

is a 5 membered heteroaryl selected from the group consisting of

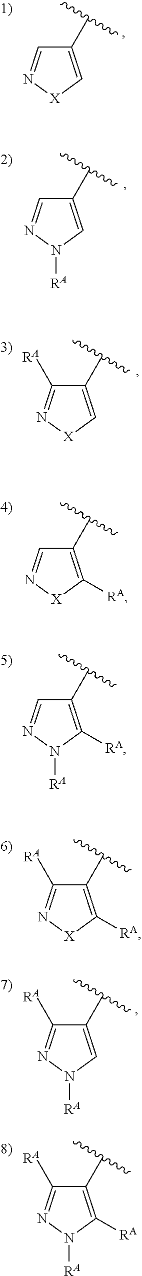

wherein X is selected from O, NH and S (e.g. X is NH).

In an embodiment, Y¹ is a bond, Y² is a bond, m is 4, R¹ and R² join together to form C₃cycloalkyl and R⁴ is H, and

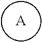

is a 5 membered heteroaryl selected from the group consisting of

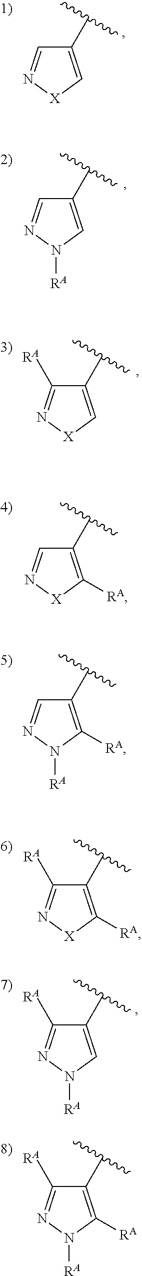

wherein X is selected from O, NH and S (e.g. X is NH).

In an embodiment, R¹ and R² join together to form C₃-C₄cycloalkyl or C₃-C₄cyclohaloalkyl, and

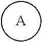

is selected from the group consisting of

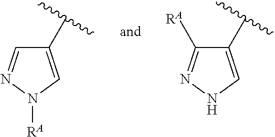

(e.g.

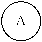

is

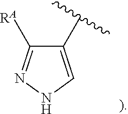

In an embodiment, R¹ and R² join together to form C₃-C₄cycloalkyl, and

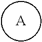

is selected from the group consisting of

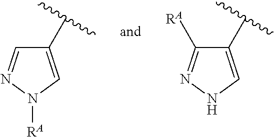

(e.g.

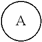

is

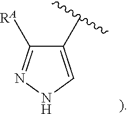

In an embodiment, R¹ and R² join together to form C₃cycloalkyl, and

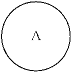

is selected from the group consisting of

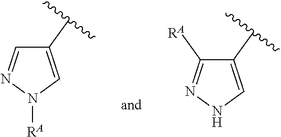

(e.g.

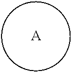

is

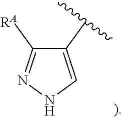

In an embodiment, R⁴ is H, and

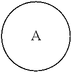

is selected from the group consisting of

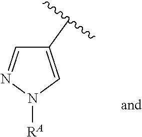

(e.g.

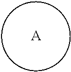

is

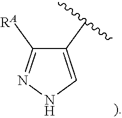

In an embodiment, R¹ and R² join together to form C₃-C₄cycloalkyl, and R⁴ is H, and

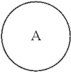

is selected from the group consisting of

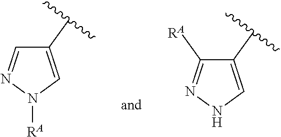

(e.g.

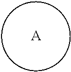

is

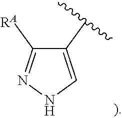

In an embodiment, R¹ and R² join together to form C₃cycloalkyl, and R⁴ is H, and

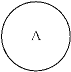

is selected from the group consisting of

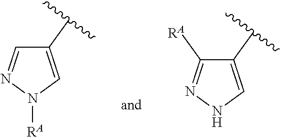

(e.g.

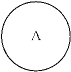

is

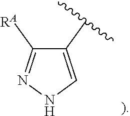

In an embodiment, m is 4 and R¹ and R² join together to form C₃-C₄cycloalkyl, and

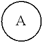

is selected from the group consisting of

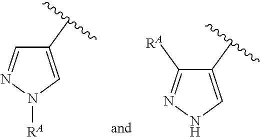

(e.g.

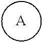

is

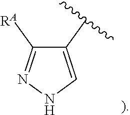

In an embodiment, m is 4 and R¹ and R² join together to form C₃cycloalkyl, and

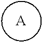

is selected from the group consisting of

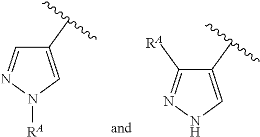

### and H (e.g.

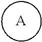

is

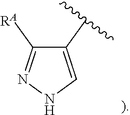

In an embodiment, m is 4 and R⁴ is H, and

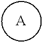

is selected from the group consisting of

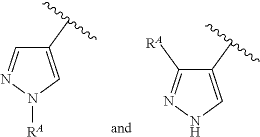

(e.g.

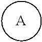

is

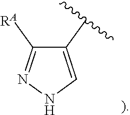

In an embodiment, m is 4, R¹ and R² join together to form C₃-C₄cycloalkyl and R⁴ is H, and

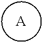

is selected from the group consisting of

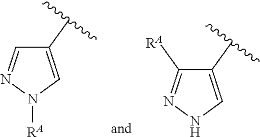

(e.g.

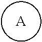

is

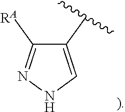

In an embodiment, m is 4, R¹ and R² join together to form C₃cycloalkyl and R⁴ is H, and

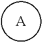

is selected from the group consisting of

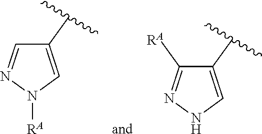

(e.g.

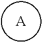

is

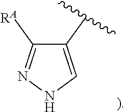

In an embodiment, Y¹ is a bond, R¹ and R² join together to form C₃-C₄cycloalkyl, and R⁴ is H, and

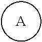

is selected from the group consisting of

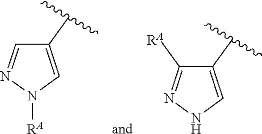

(e.g.

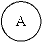

is

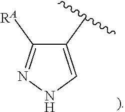

In an embodiment, Y¹ is a bond, R¹ and R² join together to form C₃cycloalkyl, and R⁴ is H, and

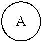

is selected from the group consisting of

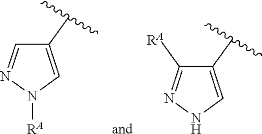

(e.g.

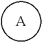

is

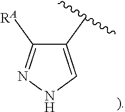

In an embodiment, Y¹ is a bond, m is 4 and R¹ and R² join together to form C₃-C₄cycloalkyl, and

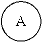

is selected from the group consisting of

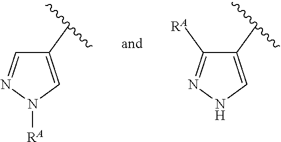

(e.g.

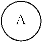

is

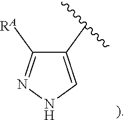

In an embodiment, Y¹ is a bond, m is 4 and R¹ and R² join together to form C₃cycloalkyl, and

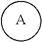

is selected from the group consisting of

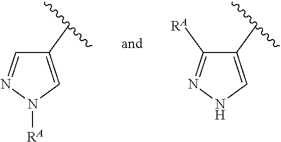

(e.g.

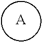

is

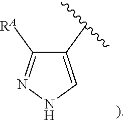

In an embodiment, Y¹ is a bond, m is 4 and R⁴ is H, and

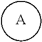

is selected from the group consisting of

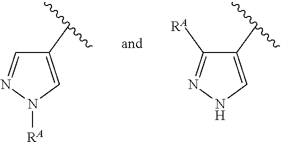

(e.g.

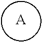

is

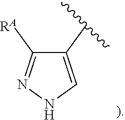

In an embodiment, Y¹ is a bond, m is 4, R¹ and R² join together to form C₃-C₄cycloalkyl and R⁴ is H, and

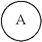

is selected from the group consisting of

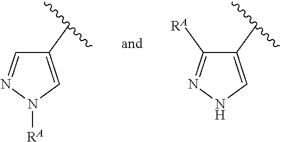

(e.g.

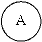

is

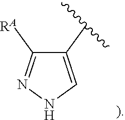

In an embodiment, Y¹ is a bond, m is 4, R¹ and R² join together to form C₃cycloalkyl and R⁴ is H, and

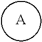

is selected from the group consisting of

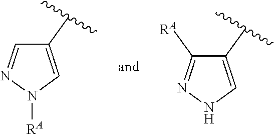

(e.g.

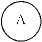

is

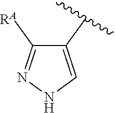

In an embodiment, Y² is a bond, R¹ and R² join together to form C₃-C₄cycloalkyl, and R⁴ is H, and

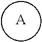

is selected from the group consisting of

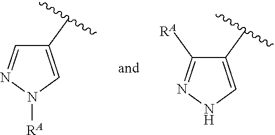

(e.g.

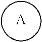

is

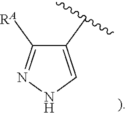

In an embodiment, Y² is a bond, R¹ and R² join together to form C₃cycloalkyl, and R⁴ is H, and

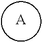

is selected from the group consisting of

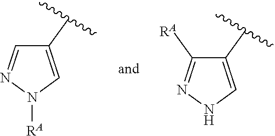

(e.g.

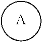

is

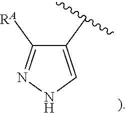

In an embodiment, Y² is a bond, m is 4 and R¹ and R² join together to form C₃-C₄cycloalkyl, and

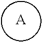

is selected from the group consisting of

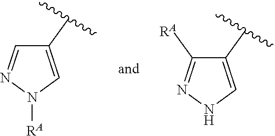

(e.g.

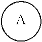

is

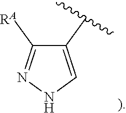

In an embodiment, Y² is a bond, m is 4 and R¹ and R² join together to form C₃cycloalkyl, and

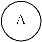

is selected from the group consisting of

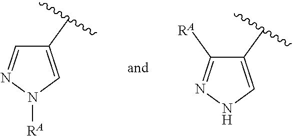

(e.g.

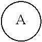

is

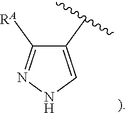

In an embodiment, Y² is a bond, m is 4 and R⁴ is H, and

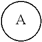

is selected from the group consisting of

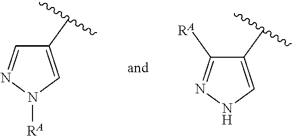

(e.g.,

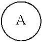

is

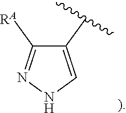

In an embodiment, Y² is a bond, m is 4, R¹ and R² join together to form C₃-C₄cycloalkyl and R⁴ is H, and

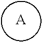

is selected from the group consisting of

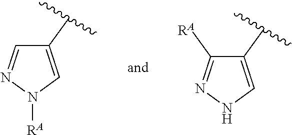

(e.g.

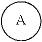

is

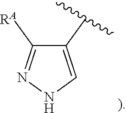

In an embodiment, Y² is a bond, m is 4, R¹ and R² join together to form C₃cycloalkyl and R⁴ is H, and

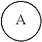

is selected from the group consisting of

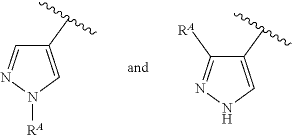

(e.g.

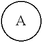

is

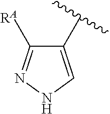

In an embodiment, Y¹ is a bond, Y² is a bond, R¹ and R² join together to form C₃-C₄cycloalkyl, and R⁴ is H, and

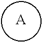

is selected from the group consisting of

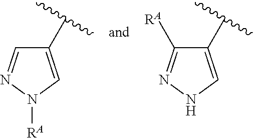

(e.g.

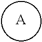

is

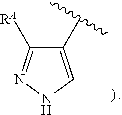

In an embodiment, Y¹ is a bond, Y² is a bond, R¹ and R² join together to form C₃cycloalkyl, and R⁴ is H, and

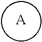

is selected from the group consisting of

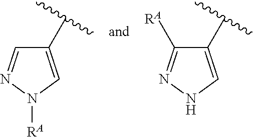

(e.g.

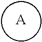

is

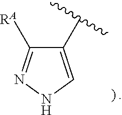

In an embodiment, Y¹ is a bond, Y² is a bond, m is 4 and R¹ and R² join together to form C₃-C₄cycloalkyl, and

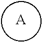

is selected from the group consisting of

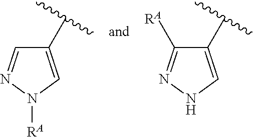

(e.g.

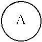

is

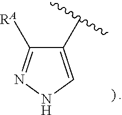

In an embodiment, Y¹ is a bond, Y² is a bond, m is 4 and R¹ and R² join together to form C₃cycloalkyl, and

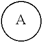

is selected from the group consisting of

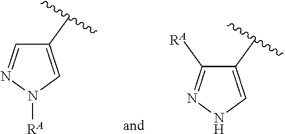

(e.g.

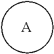

is

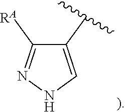

In an embodiment, Y¹ is a bond, Y² is a bond, m is 4 and R⁴ is H, and

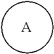

is selected from the group consisting of

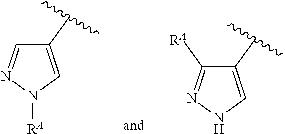

(e.g.

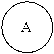

is

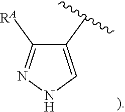

In an embodiment, Y¹ is a bond, Y² is a bond, m is 4, R¹ and R² join together to form C₃-C₄cycloalkyl and R⁴ is H, and

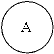

is selected from the group consisting of

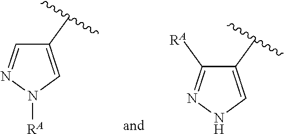

(e.g.

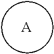

is

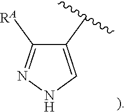

In an embodiment, Y¹ is a bond, Y² is a bond, m is 4, R¹ and R² join together to form C₃cycloalkyl and R⁴ is H, and

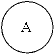

is selected from the group consisting of

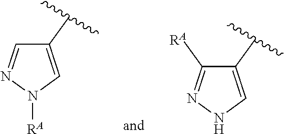

(e.g.

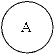

is

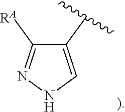

In an embodiment, the compound of formula (I) is a compound of formula (Id-I):

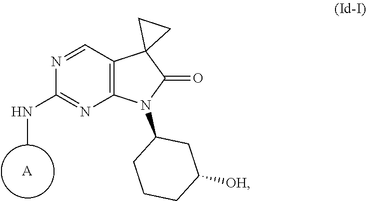

and

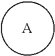

is a 5 membered heteroaryl selected from the group consisting of

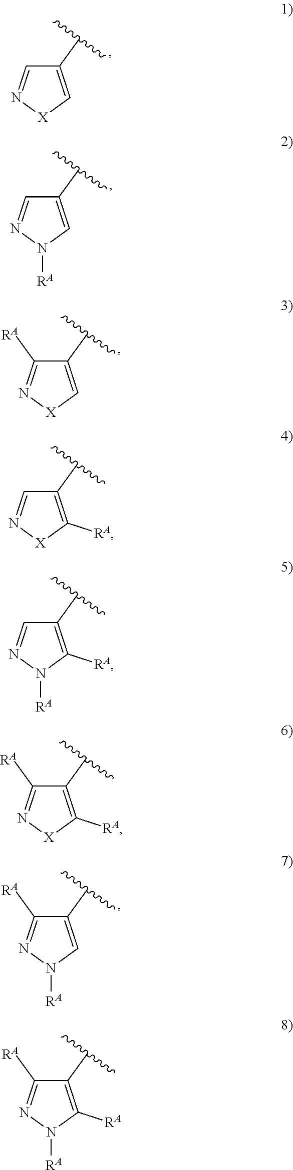

wherein X is selected from O, NH and S (e.g. X is NH).

In an embodiment, the compound of formula (I) is a compound of formula (Id-I):

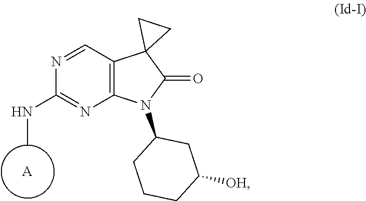

and

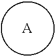

is selected from the group consisting of

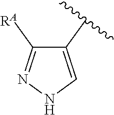

(e.g.

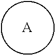

is

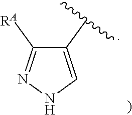

According to a second aspect of the invention, there is hereby provided a compound selected from the group consisting of:
1) 7′-((1R,3R)-3-hydroxycyclohexyl)-2′-((1-methyl-1H-pyrazol-4-yl)amino)spiro[cyclopropane-1,5′-pyrrolo[2,3-d]pyrimidin]-6′(7′H)-one;
2) 7′-((1R,3R)-3-hydroxycyclohexyl)-2′-((3-methyl-1H-pyrazol-4-yl)amino)spiro[cyclopropane-1,5′-pyrrolo[2,3-d]pyrimidin]-6′(7′H)-one;
3) 2′-((3-(difluoromethyl)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
4) methyl 4-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)-1H-pyrazole-3-carboxylate;
5) 7′-((1R,3R)-3-hydroxycyclohexyl)-2′-((3-methyl-1H-pyrazol-5-yl)amino)spiro[cyclopropane-1,5′-pyrrolo[2,3-d]pyrimidin]-6′(7′H)-one;
6) 7′-((1R,3R)-3-hydroxycyclohexyl)-2′-((3-(hydroxymethyl)-1H-pyrazol-4-yl)amino)spiro[cyclopropane-1,5′-pyrrolo[2,3-d]pyrimidin]-6′(7′H)-one;
7) 7′-((1R,5R)-5-hydroxy-3,3-dimethylcyclohexyl)-2′-((3-methyl-1H-pyrazol-4-yl)amino)spiro[cyclopropane-1,5′-pyrrolo[2,3-d]pyrimidin]-6′(7′H)-one;
8) 7′-((1R,3R)-3-hydroxycyclohexyl)-2′-((3-(tetrahydrofuran-3-yl)-1H-pyrazol-4-yl)amino)spiro[cyclopropane-1,5′-pyrrolo[2,3-d]pyrimidin]-6′(7′H)-one;
9) 7′-((1R,3R)-3-hydroxycyclohexyl)-2′-((4,5,6,7-tetrahydropyrazolo[1,5-a]pyrazin-3-yl)amino)spiro[cyclopropane-1,5′-pyrrolo[2,3-d]pyrimidin]-6′(7′H)-one;
10) 7′-((1R,3R)-3-hydroxycyclohexyl)-2′-((3-(tetrahydro-2H-pyran-2-yl)-1H-pyrazol-4-yl)amino)spiro[cyclopropane-1,5′-pyrrolo[2,3-d]pyrimidin]-6′(7′H)-one;
11) 7′-((1R,3R)-3-hydroxycyclohexyl)-2′-((2-(methoxymethyl)-1H-imidazol-5-yl)amino)spiro[cyclopropane-1,5′-pyrrolo[2,3-d]pyrimidin]-6′(7′H)-one;
12) 7′-((1R,3R)-3-hydroxycyclohexyl)-2′-((3-methoxy-1H-pyrazol-4-yl)amino)spiro[cyclopropane-1,5′-pyrrolo[2,3-d]pyrimidin]-6′(7′H)-one;
13) 2′-((3-chloro-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
14) 2′-((3-cyclopropyl-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
15) 7′-((1R,3R)-3-hydroxycyclohexyl)-2′-((3-(trifluoromethyl)-1H-pyrazol-4-yl)amino)spiro[cyclopropane-1,5′-pyrrolo[2,3-d]pyrimidin]-6′(7′H)-one;
16) 7′-((1R,3R)-3-hydroxycyclohexyl)-2′-((1-(2-methoxyethyl)-1H-pyrazol-4-yl)amino)spiro[cyclopropane-1,5′-pyrrolo[2,3-d]pyrimidin]-6′(7′H)-one;
17) 7′-(3-hydroxycycloheptyl)-2′-((3-methyl-1H-pyrazol-4-yl)amino)spiro[cyclopropane-1,5′-pyrrolo[2,3-d]pyrimidin]-6′(7′H)-one;
18) 7′-((1R,3R)-3-hydroxycycloheptyl)-2′-((3-methyl-1H-pyrazol-4-yl)amino)spiro[cyclopropane-1,5′-pyrrolo[2,3-d]pyrimidin]-6′(7′H)-one;
19) 2′-((3-chloro-1H-pyrazol-4-yl)amino)-7′-((1R,3R)-3-hydroxycycloheptyl)spiro[cyclopropane-1,5′-pyrrolo[2,3-d]pyrimidin]-6′(7′H)-one;
20) 4-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)-1H-pyrazole-3-carbonitrile;
21) 7′-((1R,3R)-3-hydroxycyclohexyl)-2′-((3-(methoxymethyl)-1H-pyrazol-4-yl)amino)spiro[cyclopropane-1,5′-pyrrolo[2,3-d]pyrimidin]-6′(7′H)-one;
22) 7′-((1R,3R)-3-hydroxycyclohexyl)-2′-((3-(2-methoxyethoxy)-1H-pyrazol-4-yl)amino)spiro[cyclopropane-1,5′-pyrrolo[2,3-d]pyrimidin]-6′(7′H)-one;
23) 2′-((3-ethoxy-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;

or a pharmaceutically acceptable salt and/or tautomer thereof.

In an embodiment, the compound is

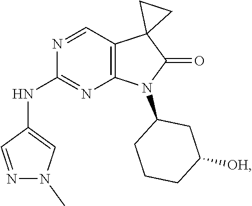 [SMILES: Cn1cc(Nc2ncc3c(n2)N([C@@H]2CCC[C@@H](O)C2)C(=O)C32CC2)cn1]

or a pharmaceutically acceptable salt and/or tautomer thereof.

In an embodiment, the compound is

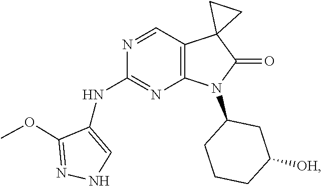 [SMILES: COc1n[nH]cc1Nc1ncc2c(n1)N([C@@H]1CCC[C@@H](O)C1)C(=O)C21CC1]

or a pharmaceutically acceptable salt and/or tautomer thereof.

In an embodiment, the compound is

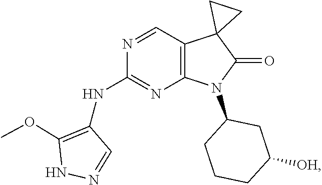 [SMILES: COc1[nH]ncc1Nc1ncc2c(n1)N([C@@H]1CCC[C@@H](O)C1)C(=O)C21CC1]

or a pharmaceutically acceptable salt and/or tautomer thereof.

In an embodiment, the compound is

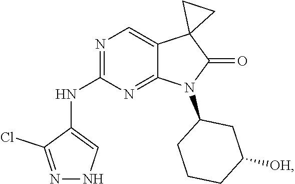 [SMILES: O=C1N([C@@H]2CCC[C@@H](O)C2)c2nc(Nc3c[nH]nc3Cl)ncc2C12CC2]

or a pharmaceutically acceptable salt and/or tautomer thereof.

In an embodiment, the compound is

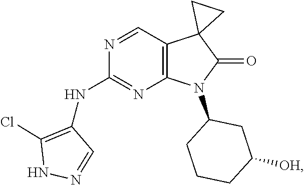 [SMILES: O=C1N([C@@H]2CCC[C@@H](O)C2)c2nc(Nc3cn[nH]c3Cl)ncc2C12CC2]

or a pharmaceutically acceptable salt and/or tautomer thereof.

In an embodiment, the compound is

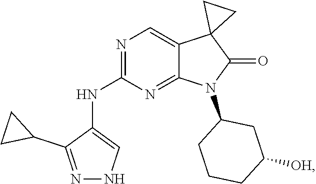 [SMILES: O=C1N([C@@H]2CCC[C@@H](O)C2)c2nc(Nc3c[nH]nc3C3CC3)ncc2C12CC2]

or a pharmaceutically acceptable salt and/or tautomer thereof.

In an embodiment, the compound is

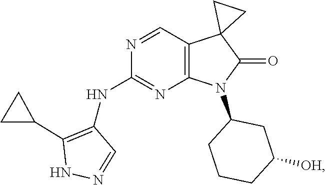 [SMILES: O=C1N([C@@H]2CCC[C@@H](O)C2)c2nc(Nc3cn[nH]c3C3CC3)ncc2C12CC2]

or a pharmaceutically acceptable salt and/or tautomer thereof.

In an embodiment, the compound is

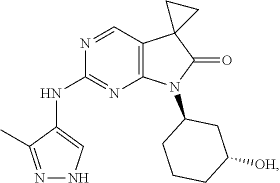 [SMILES: Cc1n[nH]cc1Nc1ncc2c(n1)N([C@@H]1CCC[C@@H](O)C1)C(=O)C21CC1]

or a pharmaceutically acceptable salt and/or tautomer thereof.

In an embodiment, the compound is

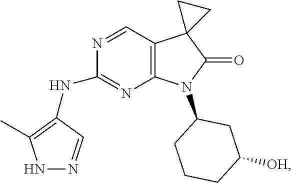 [SMILES: Cc1[nH]ncc1Nc1ncc2c(n1)N([C@@H]1CCC[C@@H](O)C1)C(=O)C21CC1]

or a pharmaceutically acceptable salt and/or tautomer thereof.

In an embodiment, the compound is

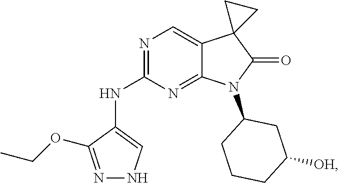 [SMILES: CCOc1n[nH]cc1Nc1ncc2c(n1)N([C@@H]1CCC[C@@H](O)C1)C(=O)C21CC1]

or a pharmaceutically acceptable salt and/or tautomer thereof.

In an embodiment, the compound is

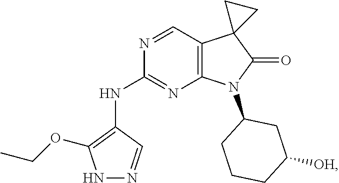 [SMILES: CCOc1[nH]ncc1Nc1ncc2c(n1)N([C@@H]1CCC[C@@H](O)C1)C(=O)C21CC1]

or a pharmaceutically acceptable salt and/or tautomer thereof.

According to a third aspect of the invention there is hereby provided a pharmaceutical composition comprising the compound or pharmaceutically acceptable salt and/or tautomer thereof according to the first of the second aspect of the invention and one or more pharmaceutically acceptable carriers.

According to a fourth aspect of the invention there is hereby provided a combination comprising the compound or pharmaceutically acceptable salt and/or tautomer thereof according to the first or the second aspect of the invention, and one or more therapeutically active agents.

According to a fifth aspect of the invention there is hereby provided a method of modulating CDK2 activity in a subject comprising administering to the subject a therapeutically effective amount of the compound or pharmaceutically acceptable salt and/or tautomer thereof according to the first or the second aspect of the invention.

According to a sixth aspect of the invention there is hereby provided a method of treating cancer comprising administering to a subject in need thereof a therapeutically effective amount of the compound or pharmaceutically acceptable salt and/or tautomer thereof according to the first or the second aspect of the invention.

According to a seventh aspect of the invention there is hereby provided a compound or pharmaceutically acceptable salt and/or tautomer thereof according to the first or the second aspect of the invention for use as a medicament.

According to an eighth aspect of the invention there is hereby provided a compound or pharmaceutically acceptable salt and/or tautomer thereof according to the first or the second aspect of the invention for use in the treatment of cancer.

According to a ninth aspect of the invention there is hereby provided use of the compound or pharmaceutically acceptable salt and/or tautomer thereof according to the first or the second aspect of the invention in the treatment of cancer.

According to a tenth aspect of the invention there is hereby provided use of the compound or pharmaceutically acceptable salt and/or tautomer thereof according to the first or the second aspect of the invention in the manufacture of a medicament for the treatment of cancer.

In an embodiment (of the sixth, eighth, ninth or tenth aspects of the invention), the cancer is selected from ovarian cancer, gastric cancer, uterine cancer, breast cancer (e.g. ER+ breast cancer, e.g. ER+/Her2− breast cancer), lung cancer and endometrial cancer.

In an embodiment (of the sixth, eighth, ninth or tenth aspects of the invention), the cancer is a cyclin E amplified cancer.

The invention therefore provides the following numbered embodiments. It will be recognized that features specified in each embodiment may be combined with other specified features to provide further embodiments of the present invention.

Embodiment 1. A compound according to formula (I),

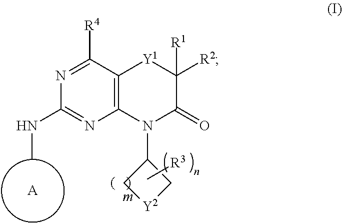

wherein:
Y¹ is a bond or CH₂ (e.g. Y¹ is a bond);
Y² is a bond, O, NR⁵ or CR⁶R⁷ (e.g. Y² is a bond);
R¹ and R² are each independently selected from the group consisting of H, halo, C₁-C₆alkyl and C₁-C₆haloalkyl, or R¹ and R² join together to form C₃-C₄cycloalkyl or C₃-C₄cyclohaloalkyl (e.g. R¹ and R² join together to form C₃-C₄cycloalkyl, e.g. R¹ and R² join together to form C₃cycloalkyl);
each R³ is independently selected from the group consisting of hydroxyl, halo, C₁-C₆alkyl and C₁-C₆haloalkyl;
R⁴ is selected from the group consisting of H, halo, C₁-C₆alkyl and C₁-C₆haloalkyl (e.g. R⁴ is H);
R⁵ is selected from the group consisting of H, C₁-C₆alkyl, C(═O)—C₁-C₆alkyl or C(═O)—O—C₁-C₆alkyl;
R⁶ and R⁷ join together to form, together with the carbon atom to which they are mutually attached, a C₃-C₆cycloalkyl or a 3-6 membered heterocyclyl comprising 1-3 heteroatoms independently selected from the group consisting of O, N and S, wherein said C₃-C₆cycloalkyl or 3-6 membered heterocyclyl is substituted with 0-3 substituents R⁸;
each R⁸ is independently selected from the group consisting of C₁-C₆alkyl, C(═O)C₁-C₆alkyl, halo, C₁-C₆haloalkyl, S—C₁-C₆alkyl, SO—C₁-C₆alkyl, SO₂—C₁-C₆alkyl, cyano, hydroxyl, or wherein two R⁸ substituents on the same ring atom join together to form ═O.
n is 0 to 3 (e.g. n is 1, e.g. n is 1 and R³ is hydroxyl);
m is 1 to 5 (e.g. m is 4);

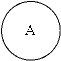

is a 5 membered heteroaryl comprising 1 to 3 heteroatoms independently selected from N, O and S (e.g. 2 heteroatoms independently selected from N, O and S, e.g. 2 heteroatoms which are both N), said 5 membered heteroaryl being substituted with 0 to 3 substituents R^{A};
each R^{A} is independently *L¹-X¹, wherein * indicates the point of attachment to

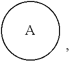

each L¹ is independently selected from bond, O, S, SO, SO₂, C≡C, C(═O), *C(═O)—O**, C₁-C₆alkylene, C₁-C₆haloalkylene, *O—C₁-C₆alkylene**, *O—C₁-C₆haloalkylene**, *O—C₁-C₆hydroxyalkylene**, C₁-C₆alkylene-O—C₁-C₆alkylene, *O—C₃-C₆cycloalkylene**, *O-3-6 membered heterocyclylene**, C₁-C₆hydroxyalkylene, C₃-C₆cycloalkylene, 3-6 membered heterocyclylene (e.g. comprising 1 heteroatom which is O), O—C₁-C₆alkylene-O, *O—C₁-C₆alkylene-O—C₃-C₆cycloalkylene** and *O—C₁-C₆alkylene-O-3-6 membered heterocyclylene**, wherein * indicates the point of attachment to

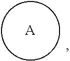

and ** indicates the point of attachment to X¹;
and each X¹ is independently selected from H, halo, cyano, hydroxyl, C₁-C₆alkyl, C(═O)—C₁-C₆alkyl, C₁-C₆haloalkyl, C₃-C₆cycloalkyl substituted by 0-3 R⁸ groups, O—C₁-C₆alkyl, S—C₁-C₆alkyl, S(O)—C₁-C₆alkyl, S(O)₂—C₁-C₆alkyl, N(C₁-C₆alkyl)₂, C(═O)N(C₁-C₆alkyl)₂, C₁-C₆hydroxyalkyl, 3-6 membered heterocyclyl (e.g. comprising 1 or 2 heteroatoms independently selected from O, N and S) substituted by 0-3 R⁸ groups, 5-10 membered heteroaryl (e.g. 5, 6, 9 or 10 membered heteroaryl) comprising 1-4 heteroatoms independently selected from O, N and S substituted by 0-3 R⁸ groups, 5-10 membered partially saturated heterocyclyl comprising 1 to 4 heteroatoms independently selected from the group consisting of O, N and S substituted by 0-3 R⁸ groups, 7-10 membered spiroheterocyclyl comprising 1 or 2 heteroatoms independently selected from O, N and S substituted by 0-3 R⁸ groups and C₇-C₁₀spirocycloalkyl substituted by 0-3 R⁸ groups; or
two R^{A} substituents located on adjacent ring atoms join together to form with said adjacent ring atoms a 4 to 6 membered heterocyclyl comprising 1 to 3 heteroatoms independently selected from N, O and S, with the proviso that at least one heteroatom is N;
or a pharmaceutically acceptable salt and/or tautomer thereof.

Embodiment 2. The compound or pharmaceutically acceptable salt and/or tautomer thereof according to Embodiment 1, wherein Y¹ is a bond.

Embodiment 3. The compound or pharmaceutically acceptable salt and/or tautomer thereof according to Embodiment 1 or Embodiment 2, wherein Y² is a bond.

Embodiment 4. The compound or pharmaceutically acceptable salt and/or tautomer thereof according to any one of the preceding Embodiments, wherein m is 4.

Embodiment 5. The compound or pharmaceutically acceptable salt and/or tautomer thereof according to any one of the preceding Embodiments, wherein n is 1 to 3, e.g. n is 1.

Embodiment 6. The compound or pharmaceutically acceptable salt and/or tautomer thereof according to Embodiment 5, wherein at least one R³ is OH.

Embodiment 7. The compound or pharmaceutically acceptable salt and/or tautomer thereof according to Embodiment 1, wherein the compound of formula (I) is a compound of formula (Ia):

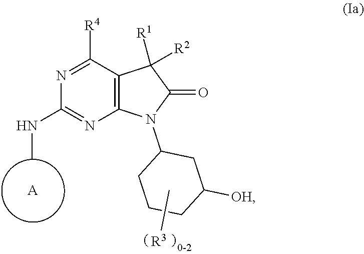

wherein R¹, R², R³, R⁴ and

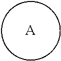

are as defined in Embodiment 1.

Embodiment 7a. The compound or pharmaceutically acceptable salt and/or tautomer thereof according to Embodiment 7, wherein the compound of formula (Ia) is a compound of formula (Ia-I):

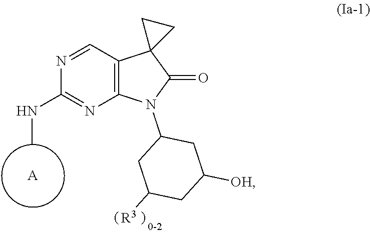

wherein R³ and

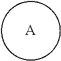

are as defined in Embodiment 1.

Embodiment 8. The compound or pharmaceutically acceptable salt and/or tautomer thereof according to Embodiment 1, wherein the compound of formula (I) is a compound of formula (Ib):

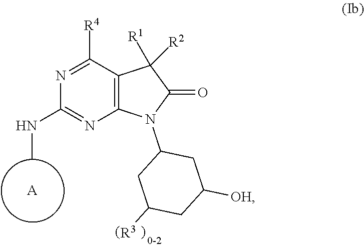

wherein R¹, R², R³, R⁴ and

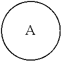

are as defined in Embodiment 1.

Embodiment 8a. The compound or pharmaceutically acceptable salt and/or tautomer thereof according to Embodiment 8, wherein the compound of formula (Ib) is a compound of formula (Ib-I):

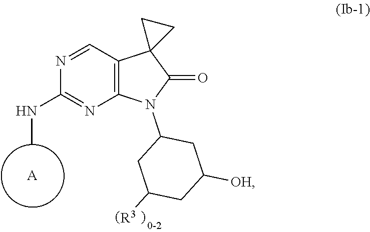

wherein R³ and

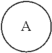

are as defined in Embodiment 1.

Embodiment 9. The compound or pharmaceutically acceptable salt and/or tautomer thereof according Embodiment 1, wherein the compound of formula (I) is a compound of formula (Ic):

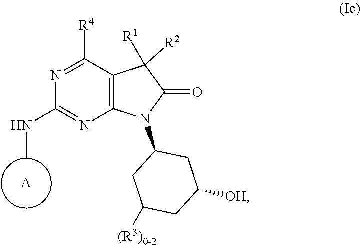

wherein R¹, R², R³, R⁴ and

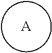

are as defined in Embodiment 1.

Embodiment 9a. The compound or pharmaceutically acceptable salt and/or tautomer thereof according to Embodiment 9, wherein the compound of formula (Ic) is a compound of formula (Ic-I):

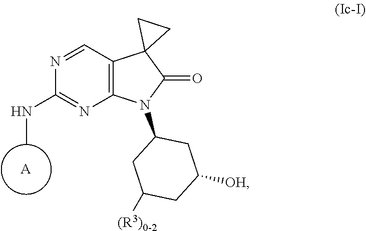

wherein R³ and

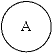

are as defined in Embodiment 1.

Embodiment 10. The compound or pharmaceutically acceptable salt and/or tautomer thereof according to Embodiment 1, wherein the compound of formula (I) is a compound of formula (Id):

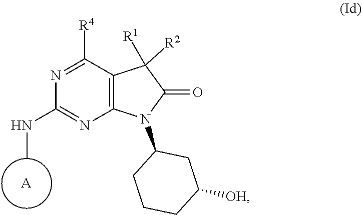

wherein R¹, R², R⁴ and

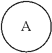

are as defined in Embodiment 1.

Embodiment 10a. The compound or pharmaceutically acceptable salt and/or tautomer thereof according to Embodiment 10, wherein the compound of formula (Id) is a compound of formula (Id-I):

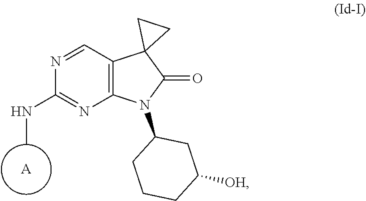

wherein

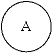

is as defined in Embodiment 1.

Embodiment 11. The compound or pharmaceutically acceptable salt and/or tautomer thereof according to any one of the preceding Embodiments, wherein R¹ and R² join together to form C₃-C₄cycloalkyl or C₃-C₄cyclohaloalkyl.

Embodiment 12. The compound or pharmaceutically acceptable salt and/or tautomer thereof according to Embodiment 11, wherein R¹ and R² join together to form C₃-C₄cycloalkyl.

Embodiment 13. The compound or pharmaceutically acceptable salt and/or tautomer thereof according to Embodiment 12, wherein R¹ and R² join together to form C₃cycloalkyl.

Embodiment 14. The compound or pharmaceutically acceptable salt and/or tautomer thereof according to any one of the preceding Embodiments, wherein R⁴ is H.

Embodiment 15. The compound or pharmaceutically acceptable salt and/or tautomer thereof according to any one of the preceding Embodiments, wherein

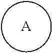

is a 5 membered heteroaryl comprising 2 heteroatoms independently selected from N, O and S, said 5 membered heteroaryl being substituted with 0 to 3 substituents R^{A}, wherein R^{A} is as defined in any one of the preceding Embodiments.

Embodiment 16. The compound or pharmaceutically acceptable salt and/or tautomer thereof according to Embodiment 15, wherein

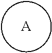

is a 5 membered heteroaryl comprising 2 heteroatoms independently selected from N and O, said 5 membered heteroaryl being substituted with 0 to 3 substituents R^{A}, wherein R^{A} is as defined in any one of the preceding Embodiments.

Embodiment 17. The compound or pharmaceutically acceptable salt and/or tautomer thereof according to any one of Embodiments 1 to 14, wherein

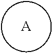

is a 5 membered heteroaryl comprising 1 to 3 heteroatoms which are each N, said 5 membered heteroaryl being substituted with 0 to 3 substituents R^{A}, wherein R^{A} is as defined in any one of the preceding Embodiments.

Embodiment 18. The compound or pharmaceutically acceptable salt and/or tautomer thereof according to any one of the preceding Embodiments, wherein

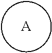

is a 5 membered heteroaryl comprising 2 heteroatoms which are each N, said 5 membered heteroaryl being substituted with 0 to 3 substituents R^{A}, wherein R^{A} is as defined in any one of the preceding Embodiments.

Embodiment 19. The compound or pharmaceutically acceptable salt and/or tautomer thereof according to any one of the preceding Embodiments, wherein

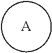

is a 5 membered heteroaryl comprising 2 heteroatoms which are each N, said 5 membered heteroaryl being substituted with 0 to 2 substituents R^{A}, wherein R^{A} is as defined in any one of the preceding Embodiments.

Embodiment 20. The compound or pharmaceutically acceptable salt and/or tautomer thereof according to any one of the preceding Embodiments, wherein

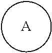

is a 5 membered heteroaryl comprising 2 heteroatoms which are each N, said 5 membered heteroaryl being substituted with 0 or 1 substituent R^{A}, wherein R^{A} is *L¹-X¹, and L¹ and X¹ are as defined in any one of the preceding Embodiments.

Embodiment 21. The compound or pharmaceutically acceptable salt and/or tautomer thereof according to any one of Embodiments 1 to 14, wherein

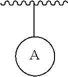

is selected from the group consisting of:

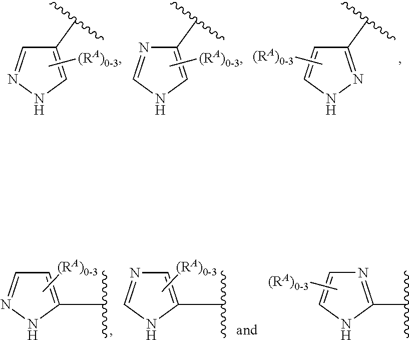

Embodiment 22. The compound or pharmaceutically acceptable salt and/or tautomer thereof according to any one of Embodiments 1 to 14 and 21, wherein

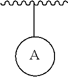

is selected from the group consisting of

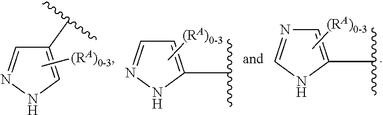

Embodiment 23. The compound or pharmaceutically acceptable salt and/or tautomer thereof according to any one of Embodiments 1 to 14, wherein

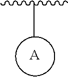

is selected from the group consisting of:

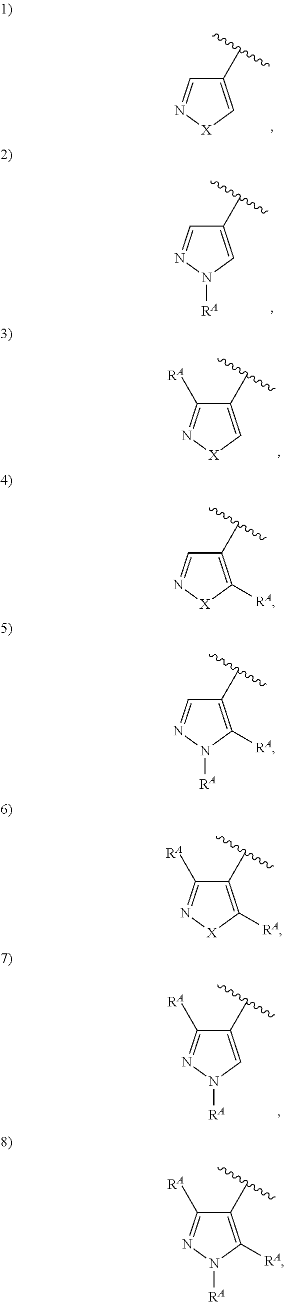

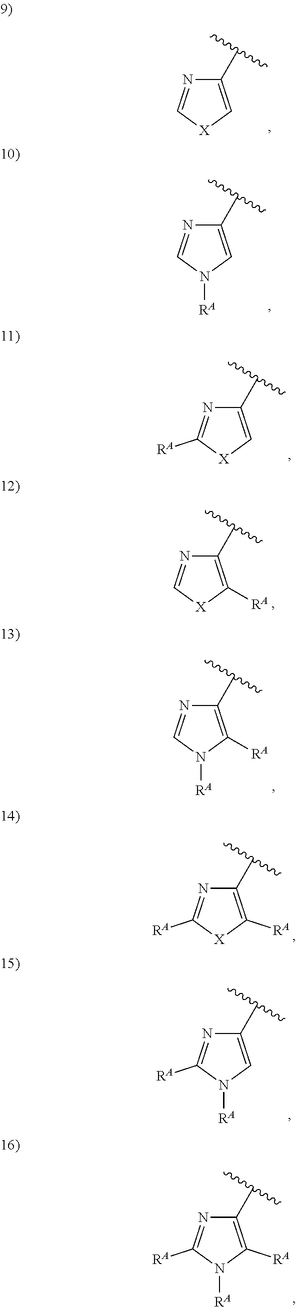

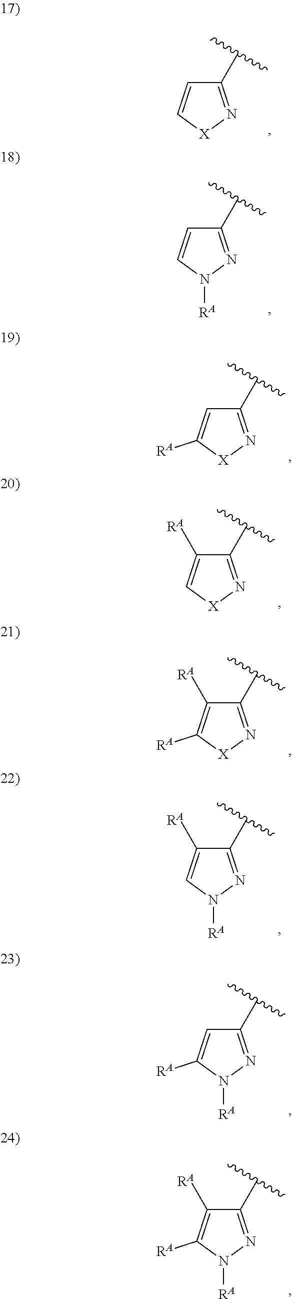

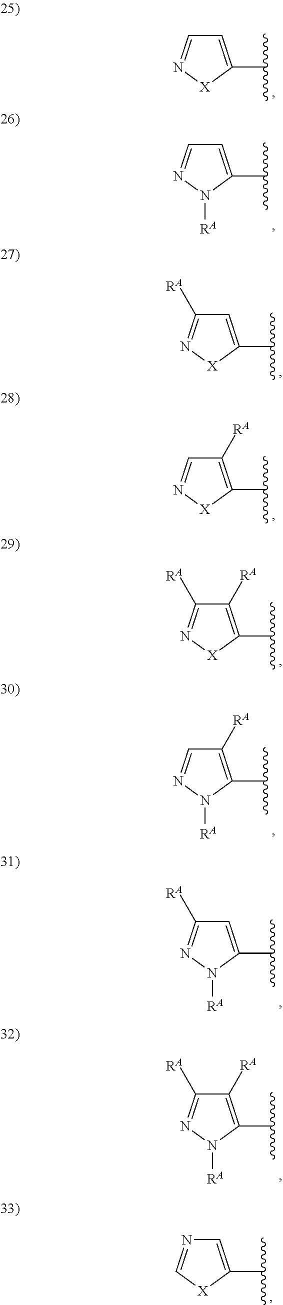

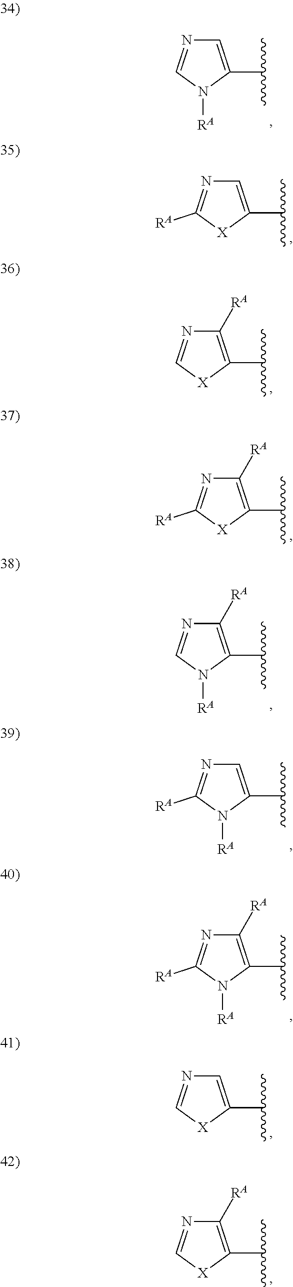

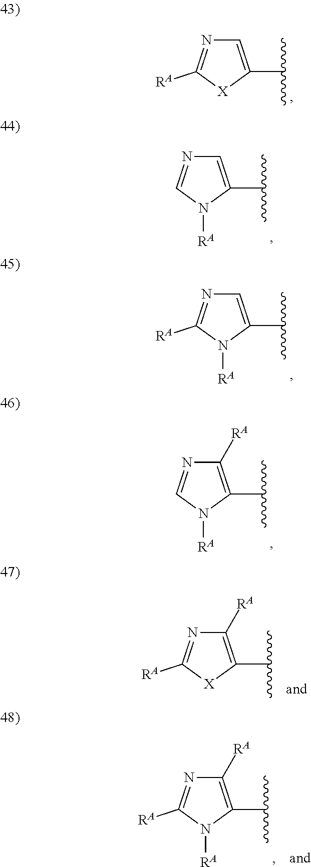

wherein X is selected from O, NH and S (e.g. X is NH).

Embodiment 24. The compound or pharmaceutically acceptable salt and/or tautomer thereof according to any one of Embodiments 1 to 14 and 22, wherein

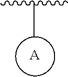

is selected from the group consisting of:

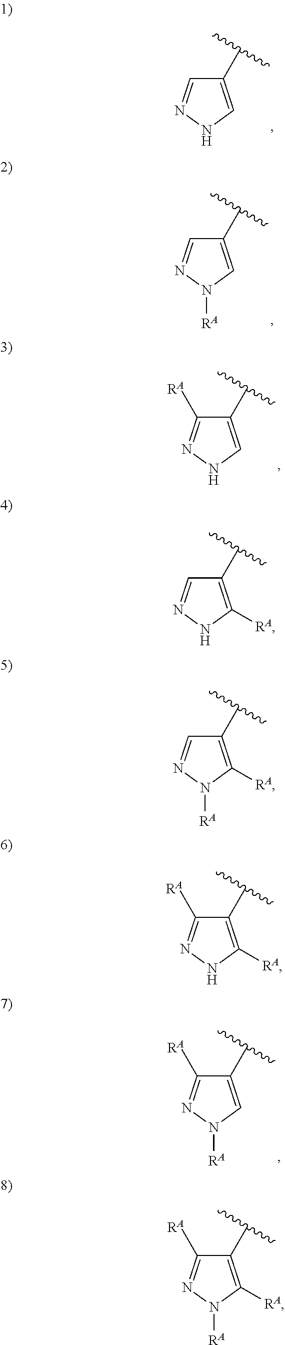

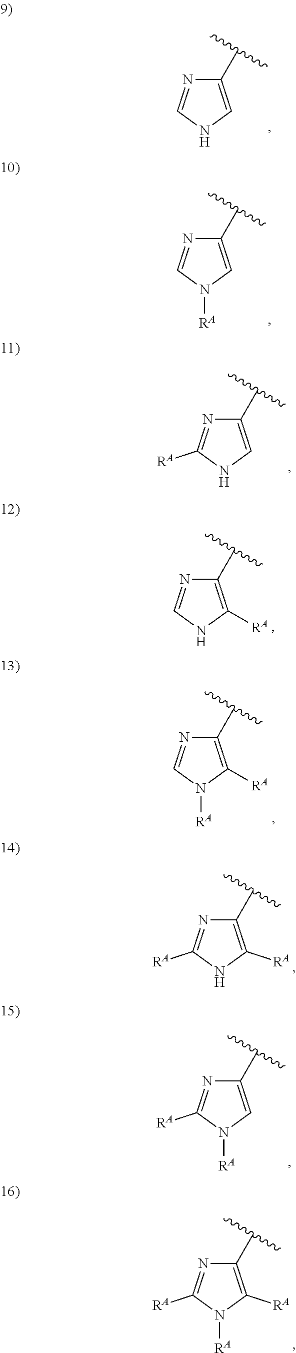

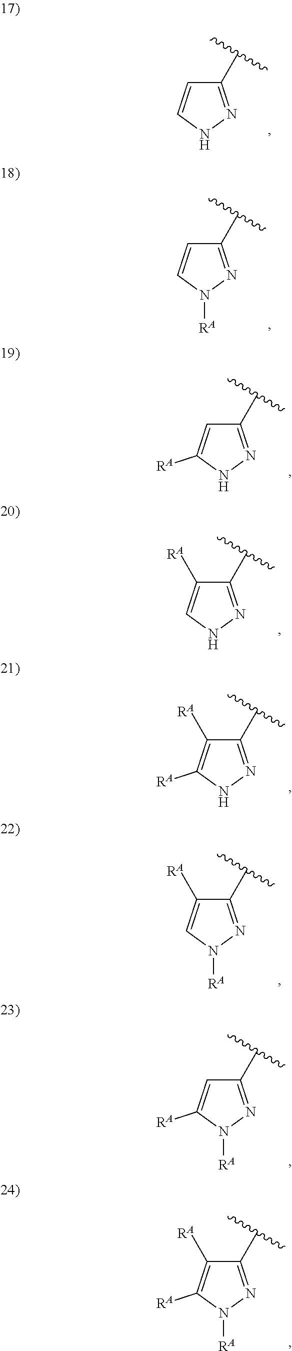

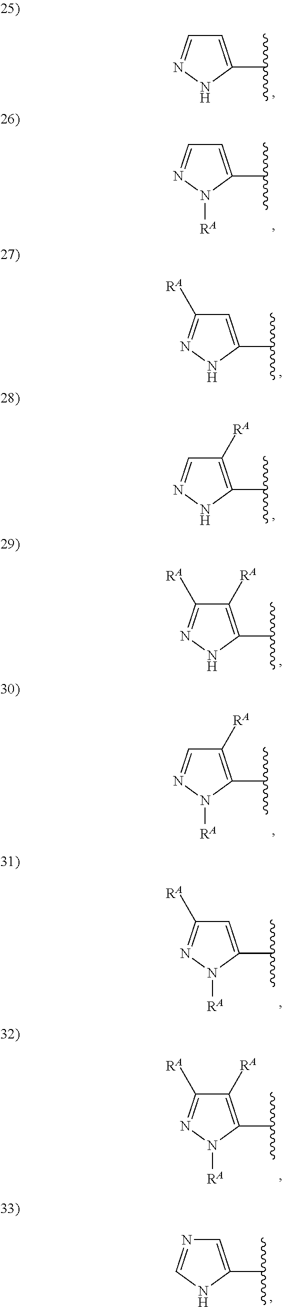

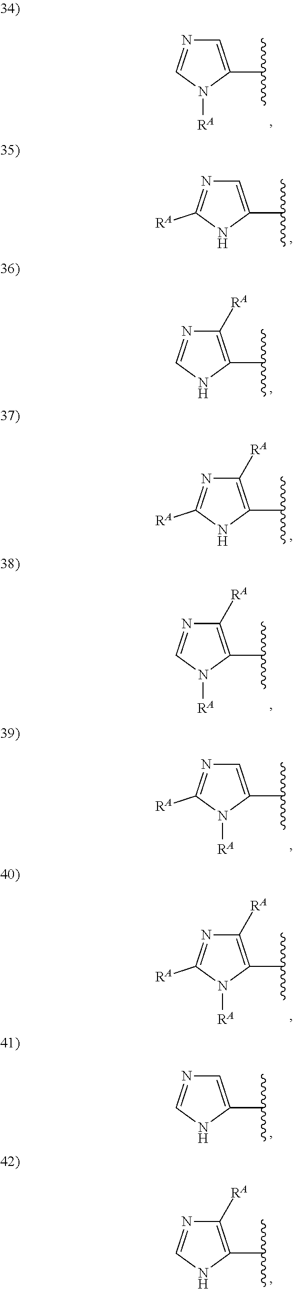

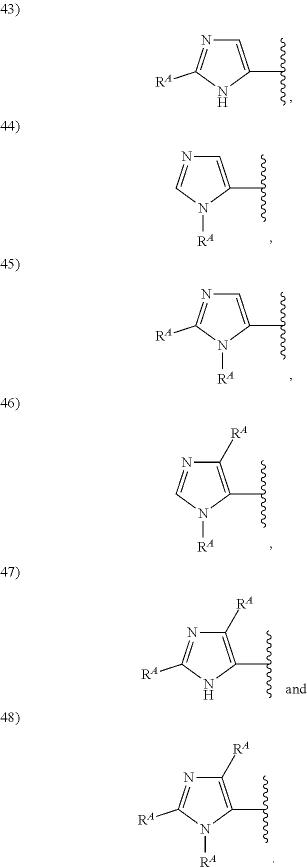

Embodiment 25. The compound or pharmaceutically acceptable salt and/or tautomer thereof according to any one of Embodiments 1 to 14 and 23, wherein

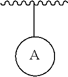

is selected from the group consisting of:

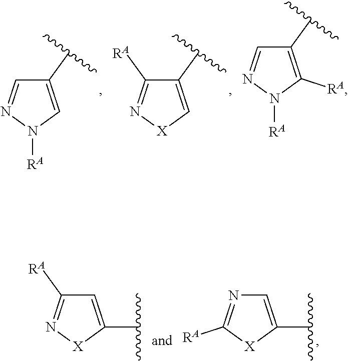

and wherein X is selected from O, NH and S.

Embodiment 26. The compound or pharmaceutically acceptable salt and/or tautomer thereof according to any one of Embodiments 1 to 14 and 25, wherein

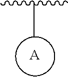

is selected from the group consisting of:

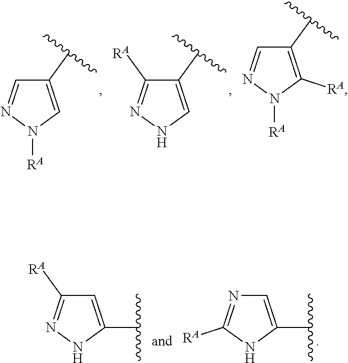

Embodiment 27. The compound or pharmaceutically acceptable salt and/or tautomer thereof according to any one of Embodiments 1 to 14 and 26, wherein:
i)

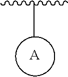

is selected from the group consisting of:

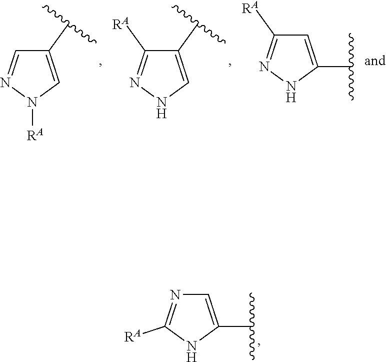

(e.g.

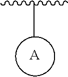

is

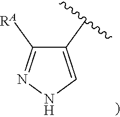

and R^{A} is selected from
a) halo,
b) cyano,
c) C₁-C₆alkyl,
d) C₁-C₆haloalkyl,
e) C₁-C₆hydroxyalkyl,
f) O—C₁-C₆alkyl,
g) C(═O)—O—C₁-C₆alkyl,
h) C₁-C₆alkylene-O—C₁-C₆alkyl,
i) O—C₁-C₆alkylene-O—C₁-C₆alkyl,
j) C₃-C₆cycloalkyl substituted by 0-3 R⁸ groups,
k) 5-10 membered heteroaryl comprising 1-4 heteroatoms independently selected from N, O and S substituted by 0-3 R⁸ groups,
l) 5-10 membered partially saturated heterocyclyl comprising 1-4 heteroatoms independently selected from N, O and S substituted by 0-3 R⁸ groups,
m) O—C₃-C₆cycloalkyl substituted by 0-3 R⁸ groups,
n) C≡C-5-10 membered heteroaryl comprising 1-4 heteroatoms independently selected from N, O and S substituted by 0-3 R⁸ groups,
o) S-5-10 membered heteroaryl comprising 1-4 heteroatoms independently selected from N, O and S substituted by 0-3 R⁸ groups,
p) C₁-C₆alkylene-5-10 membered heteroaryl comprising 1-4 heteroatoms independently selected from N, O and S substituted by 0-3 R⁸ groups,
q) O—C₁-C₆haloalkyl,
r) O—C₁-C₆alkylene-N(C₁-C₆alkyl)₂,
s) O—C₁-C₆hydroxyalkylene-O—C₁-C₆alkyl,
t) O—C₁-C₆alkylene-C₃-C₆cycloalkyl substituted by 0-3 R⁸ groups,
u) O—C₁-C₆alkylene-3-6 membered heterocyclyl comprising 1 or 2 heteroatoms independently selected from O, N and S substituted by 0-3 R⁸ groups,
v) O—C₁-C₆alkylene-C(═O)—N(C₁-C₆alkyl)₂,
w) C₁-C₆alkylene-5-10 membered partially saturated heterocyclyl comprising 1-4 heteroatoms independently selected from N, O and S substituted by 0-3 R⁸ groups,
x) O—C₁-C₆alkylene-7-10 membered spiroheterocyclyl comprising 1 or 2 heteroatoms independently selected from O, N and S substituted by 0-3 R⁸ groups,
y) O—C₁-C₆alkylene-S(O)₂—C₁-C₆alkyl,
z) O—C₁-C₆hydroxyalkyl,
aa) O—C₁-C₆alkylene-5-10 membered heteroaryl comprising 1-4 heteroatoms independently selected from N, O and S substituted by 0-3 R⁸ groups,
bb) O-5-10 membered heteroaryl comprising 1-4 heteroatoms independently selected from N, O and S substituted by 0-3 R⁸ groups,
cc) O-3-6 membered heterocyclyl comprising 1 or 2 heteroatoms independently selected from O, N and S substituted by 0-3 R⁸ groups,
dd) C≡C—C₃-C₆cycloalkyl substituted by 0-3 R⁸ groups,
ee) S—C₁-C₆haloalkyl,
ff) O—C₁-C₆alkylene-O-3-6 membered heterocyclyl comprising 1 or 2 heteroatoms independently selected from O, N and S substituted by 0-3 R⁸ groups
gg) and 3 to 6 membered heterocyclyl comprising 1 or 2 heteroatoms selected from N, O and S substituted by 0-3 R³ groups; or
ii)

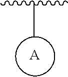

is

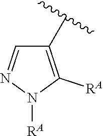

and the R^{A} substituents join together to form, with the ring atoms to which they are attached, a 4 to 6 membered heterocyclyl comprising 1 to 3 heteroatoms independently selected from N, O and S.

Embodiment 27a. The compound or pharmaceutically acceptable salt and/or tautomer thereof according to any one of Embodiments 1 to 14 and 26, wherein

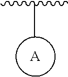

is selected from the group consisting of:
a) O—C₁-C₆haloalkylene-O—C₁-C₆alkyl;
b) hydroxyl;
c) S—C₁-C₆alkyl;
d) C₁-C₆alkylene-S—C₁-C₆alkyl; and
e) C≡C—C₁-C₆alkyl.

Embodiment 28. The compound or pharmaceutically acceptable salt and/or tautomer thereof according to Embodiment 27, wherein each R³ is independently selected from the group consisting of halo (e.g. fluoro), C₁-C₆alkyl (e.g. methyl), hydroxyl, cyano, S(O₂)—C₁-C₆alkyl (e.g. S(O₂)CH₃), C(═O)—C₁-C₆alkyl (e.g. C(═O)CH₃), O—C₁-C₆alkyl (e.g. OCH₃) and C₁-C₆haloalkyl (e.g.

C₁haloalkyl, e.g. CHF₂), or wherein two R³ substituents on the same ring atom join together to form ═O.

Embodiment 29. The compound or pharmaceutically acceptable salt and/or tautomer thereof according to Embodiment 27, wherein:
i)

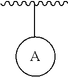

is selected from the group consisting of:

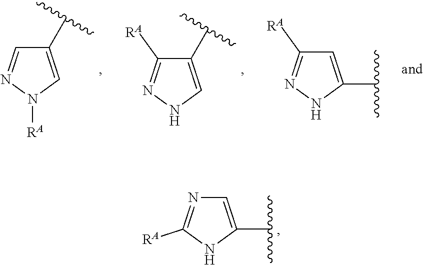

R^{A} is selected from halo, cyano, C₁-C₆alkyl, C₁-C₆haloalkyl, C₁-C₆hydroxyalkyl, O—C₁-C₆alkyl, C(═O)—O—C₁-C₆alkyl, C₁-C₆alkylene-O—C₁-C₆alkyl, O—C₁-C₆alkylene-O—C₁-C₆alkyl, C₃-C₆cycloalkyl and 3 to 6 membered heterocyclyl comprising 1 or 2 heteroatoms selected from N, O and S; or
ii)

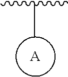

is

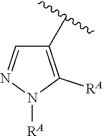

and the R^{A} substituents join together to form, with the ring atoms to which they are attached, a 4 to 6 membered heterocyclyl comprising 1 to 3 heteroatoms independently selected from N, O and S.

Embodiment 29a. The compound or pharmaceutically acceptable salt and/or tautomer thereof according to any one of Embodiments 1 to 14, wherein

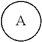

is a 5 membered heteroaryl selected from the group consisting of

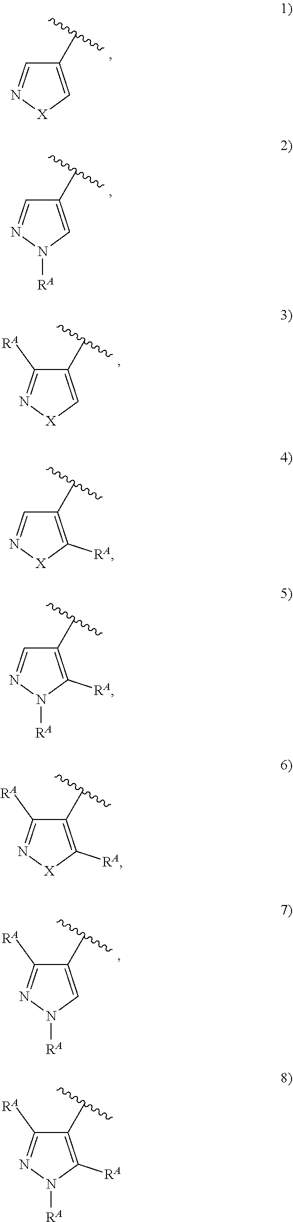

wherein X is selected from O, NH and S (e.g. X is NH).

Embodiment 29b. The compound or pharmaceutically acceptable salt and/or tautomer thereof according to any one of Embodiments 1 to 14, wherein

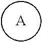

is selected from the group consisting of

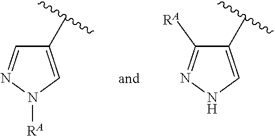

(e.g.

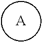

is

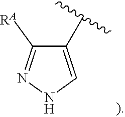

Embodiment 30. The compound or pharmaceutically acceptable salt and/or tautomer thereof according to any one of Embodiments 1 to 14, 28 and 29, wherein

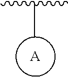

is

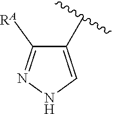

and R^{A} is CH₃, OCH₂CH₃ or OCH₃.

Embodiment 31. The compound or pharmaceutically acceptable salt and/or tautomer thereof according to any one of Embodiments 1 to 27, wherein each L¹ is independently selected from bond, O, C(═O), *C(═O)—O**, C₁-C₆alkylene, C₁-C₆haloalkylene, *O—C₁-C₆alkylene**, C₁-C₆alkylene-O—C₁-C₆alkylene, C₁-C₆hydroxyalkylene, C₃-C₆cycloalkylene, 3-6 membered heterocyclylene (e.g. comprising 1 heteroatom which is O) and O—C₁-C₆alkylene-O, wherein * indicates the point of attachment to

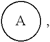

and ** indicates the point of attachment to X¹;
and each X¹ is independently selected from H, halo, cyano, hydroxyl, C₁-C₆alkyl, C₁-C₆haloalkyl, C₃-C₆cycloalkyl, O—C₁-C₆alkyl, C₁-C₆hydroxyalkyl and 3-6 membered heterocyclyl (e.g. comprising 1 heteroatom that is O).

Embodiment 31a. The compound or pharmaceutically acceptable salt and/or tautomer thereof according to Embodiment 31, wherein each L¹ is independently selected from bond, O, C(═O), *C(═O)—O**, C₁-C₂alkylene, C₁haloalkylene, *O—C₁-C₂alkylene**, C₁-C₂alkylene-O—C₁alkylene, C₁-hydroxyalkylene, C₃cycloalkylene, 5-6 membered heterocyclylene comprising 1 heteroatom which is 0 and O—C₂alkylene-O, wherein * indicates the point of attachment to

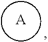

and ** indicates the point of attachment to X¹.

Embodiment 31b. The compound or pharmaceutically acceptable salt and/or tautomer thereof according to Embodiment 31 or Embodiment 31a, wherein each X¹ is independently selected from H, halo, cyano, hydroxyl, C₁-C₂alkyl (e.g. C₁alkyl), C₁haloalkyl, C₃cycloalkyl, O—C₁-C₂alkyl, C₁hydroxyalkyl and 5-6 membered heterocyclyl comprising 1 heteroatom that is O.

Embodiment 32. The compound or pharmaceutically acceptable salt and/or tautomer thereof according to any one of Embodiments 1 to 27 and 31, wherein R^{A} is selected from the list consisting of C₁-C₆alkyl, C₁-C₆alkylene-O—C₁-C₆alkyl, C₁-C₆haloalkyl, C(═O)—O—C₁-C₆alkyl, C₁-C₆hydroxyalkyl, 3-6 membered heteroatom comprising 1 heteroatom that is O, halo, O—C₁-C₆alkyl, C₃-C₆cycloalkyl, cyano and O—C₁-C₆alkylene-O—C₁-C₆alkyl.

Embodiment 32a. The compound or pharmaceutically acceptable salt and/or tautomer thereof according to Embodiment 32, wherein R^{A} is selected from the list consisting of CH₃, CH₂CH₂OCH₃, OCH₂CH₃, CHF₂, C(═O)—O—CH₃, CH₂OH, 5 membered heteroatom comprising 1 heteroatom that is O, 6 membered heteroatom comprising 1 heteroatom that is O, CH₂OCH₃, OCH₃, Cl, C₃cycloalkyl, CF₃, cyano, OCH₂CH₂OCH₃ and OCH₂CH₃.

Embodiment 33. A compound selected from any one of
1) 7′-((1R,3R)-3-hydroxycyclohexyl)-2′-((1-methyl-1H-pyrazol-4-yl)amino)spiro[cyclopropane-1,5′-pyrrolo[2,3-d]pyrimidin]-6′(7′H)-one;
2) 7′-((1R,3R)-3-hydroxycyclohexyl)-2′-((3-methyl-1H-pyrazol-4-yl)amino)spiro[cyclopropane-1,5′-pyrrolo[2,3-d]pyrimidin]-6′(7′H)-one;
3) 2′-((3-(difluoromethyl)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
4) methyl 4-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)-1H-pyrazole-3-carboxylate;
5) 7′-((1R,3R)-3-hydroxycyclohexyl)-2′-((3-methyl-1H-pyrazol-5-yl)amino)spiro[cyclopropane-1,5′-pyrrolo[2,3-d]pyrimidin]-6′(7′H)-one;
6) 7′-((1R,3R)-3-hydroxycyclohexyl)-2′-((3-(hydroxymethyl)-1H-pyrazol-4-yl)amino)spiro[cyclopropane-1,5′-pyrrolo[2,3-d]pyrimidin]-6′(7′H)-one;
7) 7′-((1R,5R)-5-hydroxy-3,3-dimethylcyclohexyl)-2′-((3-methyl-1H-pyrazol-4-yl)amino)spiro[cyclopropane-1,5′-pyrrolo[2,3-d]pyrimidin]-6′(7′H)-one;
8) 7′-((1R,3R)-3-hydroxycyclohexyl)-2′-((3-(tetrahydrofuran-3-yl)-1H-pyrazol-4-yl)amino)spiro[cyclopropane-1,5′-pyrrolo[2,3-d]pyrimidin]-6′(7′H)-one;
9) 7′-((1R,3R)-3-hydroxycyclohexyl)-2′-((4,5,6,7-tetrahydropyrazolo[1,5-a]pyrazin-3-yl)amino)spiro[cyclopropane-1,5′-pyrrolo[2,3-d]pyrimidin]-6′(7′H)-one;
10) 7′-((1R,3R)-3-hydroxycyclohexyl)-2′-((3-(tetrahydro-2H-pyran-2-yl)-1H-pyrazol-4-yl)amino)spiro[cyclopropane-1,5′-pyrrolo[2,3-d]pyrimidin]-6′(7′H)-one;
11) 7′-((1R,3R)-3-hydroxycyclohexyl)-2′-((2-(methoxymethyl)-1H-imidazol-5-yl)amino)spiro[cyclopropane-1,5′-pyrrolo[2,3-d]pyrimidin]-6′(7′H)-one;
12) 7′-((1R,3R)-3-hydroxycyclohexyl)-2′-((3-methoxy-1H-pyrazol-4-yl)amino)spiro[cyclopropane-1,5′-pyrrolo[2,3-d]pyrimidin]-6′(7′H)-one;
13) 2′-((3-chloro-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
14) 2′-((3-cyclopropyl-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
15) 7′-((1R,3R)-3-hydroxycyclohexyl)-2′-((3-(trifluoromethyl)-1H-pyrazol-4-yl)amino)spiro[cyclopropane-1,5′-pyrrolo[2,3-d]pyrimidin]-6′(7′H)-one;
16) 7′-((1R,3R)-3-hydroxycyclohexyl)-2′-((1-(2-methoxyethyl)-1H-pyrazol-4-yl)amino)spiro[cyclopropane-1,5′-pyrrolo[2,3-d]pyrimidin]-6′(7′H)-one;
17) 7′-(3-hydroxycycloheptyl)-2′-((3-methyl-1H-pyrazol-4-yl)amino)spiro[cyclopropane-1,5′-pyrrolo[2,3-d]pyrimidin]-6′(7′H)-one;
18) 7′-((1R,3R)-3-hydroxycycloheptyl)-2′-((3-methyl-1H-pyrazol-4-yl)amino)spiro[cyclopropane-1,5′-pyrrolo[2,3-d]pyrimidin]-6′(7′H)-one;
19) 2′-((3-chloro-1H-pyrazol-4-yl)amino)-7′-((1R,3R)-3-hydroxycycloheptyl)spiro[cyclopropane-1,5′-pyrrolo[2,3-d]pyrimidin]-6′(7′H)-one;
20) 4-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)-1H-pyrazole-3-carbonitrile;
21) 7′-((1R,3R)-3-hydroxycyclohexyl)-2′-((3-(methoxymethyl)-1H-pyrazol-4-yl)amino)spiro[cyclopropane-1,5′-pyrrolo[2,3-d]pyrimidin]-6′(7′H)-one;
22) 7′-((1R,3R)-3-hydroxycyclohexyl)-2′-((3-(2-methoxyethoxy)-1H-pyrazol-4-yl)amino)spiro[cyclopropane-1,5′-pyrrolo[2,3-d]pyrimidin]-6′(7′H)-one;
23) 2′-((3-ethoxy-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;

or a pharmaceutically acceptable salt and/or tautomer thereof.

Embodiment 33a. A compound selected from any one of:
24) 2′-((3-(((3R,4R)-4-fluorotetrahydrofuran-3-yl)oxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
25) 2′-((3-(((3S,4S)-4-fluorotetrahydrofuran-3-yl)oxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
26) 2′-((3-(((3R,4S)-4-fluorotetrahydrofuran-3-yl)oxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
27) 2′-((3-(((3S,4R)-4-fluorotetrahydrofuran-3-yl)oxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
28) 7′-((1R,3R)-3-hydroxycyclohexyl)-2′-((3-(pyrazolo[1,5-a]pyridin-3-yl)-1H-pyrazol-4-yl)amino)spiro[cyclopropane-1,5′-pyrrolo[2,3-d]pyrimidin]-6′(7′H)-one;
29) 2′-((3-(3,3-difluorocyclobutoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
30) 2′-((3-(3,3-difluorocyclobutoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
31) 2′-((5′-(difluoromethyl)-1′-methyl-1H,1′H-[3,4′-bipyrazol]-4-yl)amino)-7′-((1R,3R)-3-hydroxycyclohexyl)spiro[cyclopropane-1,5′-pyrrolo[2,3-d]pyrimidin]-6′(7′H)-one;
32) 2′-((3-((S)-2,2-difluorocyclopropyl)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
33) 2′-((3-((S)-2,2-difluorocyclopropyl)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
34) 2′-((1′-(difluoromethyl)-1H,1′H-[3,4′-bipyrazol]-4-yl)amino)-7′-((1R,3R)-3-hydroxycyclohexyl)spiro[cyclopropane-1,5′-pyrrolo[2,3-d]pyrimidin]-6′(7′H)-one;
35) 7′-((1R,3R)-3-hydroxycyclohexyl)-2′-((3-((1-methyl-1H-pyrazol-4-yl)oxy)-1H-pyrazol-4-yl)amino)spiro[cyclopropane-1,5′-pyrrolo[2,3-d]pyrimidin]-6′(7′H)-one;
36) 7′-((1R,3R)-3-hydroxycyclohexyl)-2′-((3-(pyridin-3-ylethynyl)-1H-pyrazol-4-yl)amino)spiro[cyclopropane-1,5′-pyrrolo[2,3-d]pyrimidin]-6′(7′H)-one;
37) 2′-((1′,5′-dimethyl-1H,1′H-[3,4′-bipyrazol]-4-yl)amino)-7′-((1R,3R)-3-hydroxycyclohexyl)spiro[cyclopropane-1,5′-pyrrolo[2,3-d]pyrimidin]-6′(7′H)-one;
38) 2′-((3-(cyclopropylmethoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
39) 2′-((3-(2-fluoroethoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
40) 2′-((3-cyclobutoxy-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
41) 2′-((3-(2,2-difluoroethoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
42) 7′-((1R,3R)-3-hydroxycyclohexyl)-2′-((3-(2-hydroxyethoxy)-1H-pyrazol-4-yl)amino)spiro[cyclopropane-1,5′-pyrrolo[2,3-d]pyrimidin]-6′(7′H)-one;
43) 7′-((1R,3R)-3-hydroxycyclohexyl)-2′-((3-((1-methyl-1H-pyrazol-3-yl)methoxy)-1H-pyrazol-4-yl)amino)spiro[cyclopropane-1,5′-pyrrolo[2,3-d]pyrimidin]-6′(7′H)-one;
44) 2′-((3-(2,2-difluoro-3-methoxypropoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
45) 2′-((3-((1-fluorocyclopropyl)methoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
46) 7′-((1R,3R)-3-hydroxycyclohexyl)-2′-((3-((1-(2,2,2-trifluoroethyl)azetidin-3-yl)oxy)-1H-pyrazol-4-yl)amino)spiro[cyclopropane-1,5′-pyrrolo[2,3-d]pyrimidin]-6′(7′H)-one;
47) 2′-((3-(5,6-dihydro-4H-pyrrolo[1,2-b]pyrazol-3-yl)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
48) 2′-((3-((3-fluorooxetan-3-yl)methoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
49) 7′-((1R,3R)-3-hydroxycyclohexyl)-2′-((3-(methoxy-d3)-1H-pyrazol-4-yl)amino)spiro[cyclopropane-1,5′-pyrrolo[2,3-d]pyrimidin]-6′(7′H)-one;
50) 2′-((3-(((3R,4R)-4-fluoro-1-methylpyrrolidin-3-yl)oxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
51) 2′-((3-(((3S,4S)-4-fluoro-1-methylpyrrolidin-3-yl)oxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
52) 7′-((1R,3R)-3-hydroxycyclohexyl)-2′-((3-(methylthio)-1H-pyrazol-4-yl)amino)spiro[cyclopropane-1,5′-pyrrolo[2,3-d]pyrimidin]-6′(7′H)-one;
53) 2′-((3-((2-acetyl-2-azaspiro[3.3]heptan-6-yl)methoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
54) 2′-((3-((2-oxaspiro[3.3]heptan-6-yl)methoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
55) 2′-((3-cyclopropoxy-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
56) 7′-((1R,3R)-3-hydroxycyclohexyl)-2′-((3-(2,2,2-trifluoroethoxy)-1H-pyrazol-4-yl)amino)spiro[cyclopropane-1,5′-pyrrolo[2,3-d]pyrimidin]-6′(7′H)-one;
57) 2′-((5′-chloro-1′-methyl-1H,1′H-[3,4′-bipyrazol]-4-yl)amino)-7′-((1R,3R)-3-hydroxycyclohexyl)spiro[cyclopropane-1,5′-pyrrolo[2,3-d]pyrimidin]-6′(7′H)-one;
58) 7′-((1R,3R)-3-hydroxycyclohexyl)-2′-((3-(prop-1-yn-1-yl)-1H-pyrazol-4-yl)amino)spiro[cyclopropane-1,5′-pyrrolo[2,3-d]pyrimidin]-6′(7′H)-one;
59) 7′-((1R,3R)-3-hydroxycyclohexyl)-2′-((3-(pyridin-4-yl)-1H-pyrazol-4-yl)amino)spiro[cyclopropane-1,5′-pyrrolo[2,3-d]pyrimidin]-6′(7′H)-one;
60) 7′-((1R,3R)-3-hydroxycyclohexyl)-2′-((3-(pyridin-2-yl)-1H-pyrazol-4-yl)amino)spiro[cyclopropane-1,5′-pyrrolo[2,3-d]pyrimidin]-6′(7′H)-one;
61) 7′-((1R,3R)-3-hydroxycyclohexyl)-2′-((3-(pyridin-3-yl)-1H-pyrazol-4-yl)amino)spiro[cyclopropane-1,5′-pyrrolo[2,3-d]pyrimidin]-6′(7′H)-one;
62) 2′-((1′-(difluoromethyl)-5′-methyl-1H,1′H-[3,4′-bipyrazol]-4-yl)amino)-7′-((1R,3R)-3-hydroxycyclohexyl)spiro[cyclopropane-1,5′-pyrrolo[2,3-d]pyrimidin]-6′(7′H)-one;
63) 7′-((1R,3R)-3-hydroxycyclohexyl)-2′-((3-(((R)-tetrahydrofuran-3-yl)oxy)-1H-pyrazol-4-yl)amino)spiro[cyclopropane-1,5′-pyrrolo[2,3-d]pyrimidin]-6′(7′H)-one;
64) 7′-((1R,3R)-3-hydroxycyclohexyl)-2′-((3-(pyrazolo[1,5-a]pyrazin-3-yl)-1H-pyrazol-4-yl)amino)spiro[cyclopropane-1,5′-pyrrolo[2,3-d]pyrimidin]-6′(7′H)-one;
65) 2′-((3-(((1r,3R)-3-hydroxy-3-methylcyclobutyl)methoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
66) 2′-((3-(((R)-1,4-dioxan-2-yl)methoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
67) 1-(((4-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)-1H-pyrazol-3-yl)oxy)methyl)cyclopropane-1-carbonitrile;
68) 7′-((1R,3R)-3-hydroxycyclohexyl)-2′-((3-((methylthio)methyl)-1H-pyrazol-4-yl)amino)spiro[cyclopropane-1,5′-pyrrolo[2,3-d]pyrimidin]-6′(7′H)-one;
69) 2′-((3-((S)-2,2-difluorocyclopropoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
70) 2′-((3-((R)-2,2-difluorocyclopropoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
71) 7′-((1R,3R)-3-hydroxycyclohexyl)-2′-((3-(((R)-tetrahydrofuran-3-yl)methoxy)-1H-pyrazol-4-yl)amino)spiro[cyclopropane-1,5′-pyrrolo[2,3-d]pyrimidin]-6′(7′H)-one;
72) 7′-((1R,3R)-3-hydroxycyclohexyl)-2′-((3-(((S)-tetrahydrofuran-3-yl)methoxy)-1H-pyrazol-4-yl)amino)spiro[cyclopropane-1,5′-pyrrolo[2,3-d]pyrimidin]-6′(7′H)-one;
73) 7′-((1R,3R)-3-hydroxycyclohexyl)-2′-((3-(4,5,6,7-tetrahydropyrazolo[1,5-a]pyridin-3-yl)-1H-pyrazol-4-yl)amino)spiro[cyclopropane-1,5′-pyrrolo[2,3-d]pyrimidin]-6′(7′H)-one;
74) 7′-((1R,3R)-3-hydroxycyclohexyl)-2′-((3-((tetrahydro-2H-pyran-4-yl)oxy)-1H-pyrazol-4-yl)amino)spiro[cyclopropane-1,5′-pyrrolo[2,3-d]pyrimidin]-6′(7′H)-one;
75) 2′-((3-((-4-fluorotetrahydrofuran-3-yl)methoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
76) 2′-((3-((4,4-difluorotetrahydrofuran-3-yl)methoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
77) 2′-((3-hydroxy-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
78) 7′-((1R,3R)-3-hydroxycyclohexyl)-2′-((3-(((S)-tetrahydrofuran-3-yl)oxy)-1H-pyrazol-4-yl)amino)spiro[cyclopropane-1,5′-pyrrolo[2,3-d]pyrimidin]-6′(7′H)-one;
79) 7′-((1R,3R)-3-hydroxycyclohexyl)-2′-((1′-methyl-1H,1′H-[3,3′-bipyrazol]-4-yl)amino)spiro[cyclopropane-1,5′-pyrrolo[2,3-d]pyrimidin]-6′(7′H)-one;
80) 7′-((1R,3R)-3-hydroxycyclohexyl)-2′-((1′-methyl-1H,1′H-[3,4′-bipyrazol]-4-yl)amino)spiro[cyclopropane-1,5′-pyrrolo[2,3-d]pyrimidin]-6′(7′H)-one;
81) 7′-((1R,3R)-3-hydroxycyclohexyl)-2′-((3-(pyrazolo[1,5-a]pyrimidin-3-yl)-1H-pyrazol-4-yl)amino)spiro[cyclopropane-1,5′-pyrrolo[2,3-d]pyrimidin]-6′(7′H)-one;
82) 7′-((1R,3R)-3-hydroxycyclohexyl)-2′-((3-((1r,3R)-3-methoxycyclobutoxy)-1H-pyrazol-4-yl)amino)spiro[cyclopropane-1,5′-pyrrolo[2,3-d]pyrimidin]-6′(7′H)-one;
83) 2′-((3-(1,3,4-oxadiazol-2-yl)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
84) 7′-((1R,3R)-3-hydroxycyclohexyl)-2′-((1′-methyl-5′-(trifluoromethyl)-1H,1′H-[3,4′-bipyrazol]-4-yl)amino)spiro[cyclopropane-1,5′-pyrrolo[2,3-d]pyrimidin]-6′(7′H)-one;
85) 2′-((3-(1-(fluoromethyl)cyclopropoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;

or a pharmaceutically acceptable salt and/or tautomer thereof.

Embodiment 34. The compound according to Embodiment 1, wherein the compound is

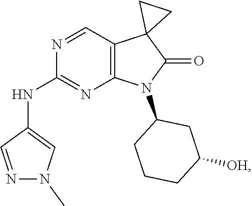 [SMILES: Cn1cc(Nc2ncc3c(n2)N([C@@H]2CCC[C@@H](O)C2)C(=O)C32CC2)cn1]

or a pharmaceutically acceptable salt and/or tautomer thereof.

Embodiment 35. The compound according to Embodiment 1, wherein the compound is

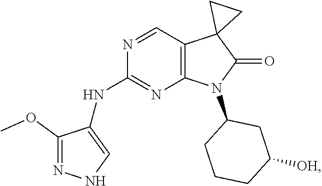 [SMILES: COc1n[nH]cc1Nc1ncc2c(n1)N([C@@H]1CCC[C@@H](O)C1)C(=O)C21CC1]

or a pharmaceutically acceptable salt and/or tautomer thereof.

Embodiment 35a. The compound according to Embodiment 1, wherein the compound is

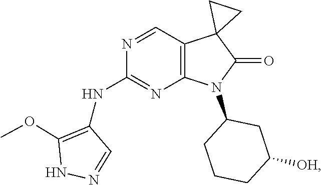 [SMILES: COc1[nH]ncc1Nc1ncc2c(n1)N([C@@H]1CCC[C@@H](O)C1)C(=O)C21CC1]

or a pharmaceutically acceptable salt and/or tautomer thereof.

Embodiment 36. The compound according to Embodiment 1, wherein the compound is

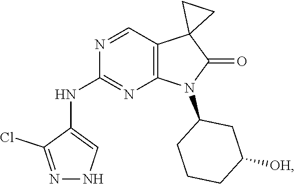 [SMILES: O=C1N([C@@H]2CCC[C@@H](O)C2)c2nc(Nc3c[nH]nc3Cl)ncc2C12CC2]

or a pharmaceutically acceptable salt and/or tautomer thereof.

Embodiment 36a. The compound according to Embodiment 1, wherein the compound is

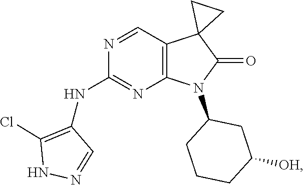 [SMILES: O=C1N([C@@H]2CCC[C@@H](O)C2)c2nc(Nc3cn[nH]c3Cl)ncc2C12CC2]

or a pharmaceutically acceptable salt and/or tautomer thereof.

Embodiment 37. The compound according to Embodiment 1, wherein the compound is

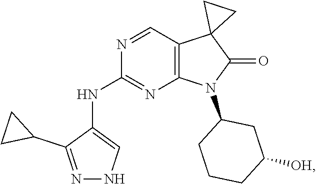 [SMILES: O=C1N([C@@H]2CCC[C@@H](O)C2)c2nc(Nc3c[nH]nc3C3CC3)ncc2C12CC2]

or a pharmaceutically acceptable salt and/or tautomer thereof.

Embodiment 37a. The compound according to Embodiment 1, wherein the compound is

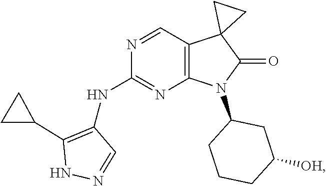 [SMILES: O=C1N([C@@H]2CCC[C@@H](O)C2)c2nc(Nc3cn[nH]c3C3CC3)ncc2C12CC2]

or a pharmaceutically acceptable salt and/or tautomer thereof.

Embodiment 38. The compound according to Embodiment 1, wherein the compound is

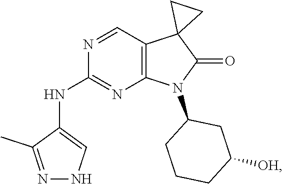 [SMILES: Cc1n[nH]cc1Nc1ncc2c(n1)N([C@@H]1CCC[C@@H](O)C1)C(=O)C21CC1]

or a pharmaceutically acceptable salt and/or tautomer thereof.

Embodiment 38a. The compound according to Embodiment 1, wherein the compound is

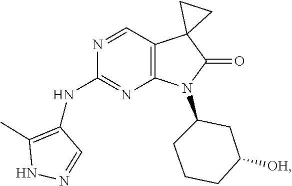 [SMILES: Cc1[nH]ncc1Nc1ncc2c(n1)N([C@@H]1CCC[C@@H](O)C1)C(=O)C21CC1]

or a pharmaceutically acceptable salt and/or tautomer thereof.

Embodiment 39. The compound according to Embodiment 1, wherein the compound is

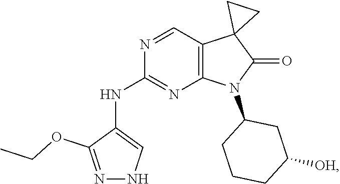 [SMILES: CCOc1n[nH]cc1Nc1ncc2c(n1)N([C@@H]1CCC[C@@H](O)C1)C(=O)C21CC1]

or a pharmaceutically acceptable salt and/or tautomer thereof.

Embodiment 39a. The compound according to Embodiment 1, wherein the compound is

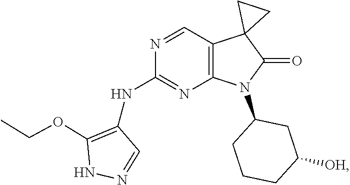 [SMILES: CCOc1[nH]ncc1Nc1ncc2c(n1)N([C@@H]1CCC[C@@H](O)C1)C(=O)C21CC1]

or a pharmaceutically acceptable salt and/or tautomer thereof.

Embodiment 40. A pharmaceutical composition comprising the compound or pharmaceutically acceptable salt and/or tautomer thereof according to any one of the preceding Embodiments and one or more pharmaceutically acceptable carriers.

Embodiment 41. A combination comprising the compound or pharmaceutically acceptable salt and/or tautomer thereof according to any one of Embodiments 1 to 39a, and one or more therapeutically active agents.

Embodiment 42. A method of modulating CDK2 activity in a subject comprising administering to the subject a therapeutically effective amount of the compound or pharmaceutically acceptable salt and/or tautomer thereof according to any one of Embodiments 1 to 39a.

Embodiment 43. A method of treating cancer comprising administering to a subject in need thereof a therapeutically effective amount of the compound or pharmaceutically acceptable salt and/or tautomer thereof according to any one of Embodiments 1 to 39a.

Embodiment 44. A compound or pharmaceutically acceptable salt and/or tautomer thereof according to any one of Embodiments 1 to 39a for use as a medicament.

Embodiment 45. A compound or pharmaceutically acceptable salt and/or tautomer thereof according to any one of Embodiments 1 to 39a for use in the treatment of cancer.

Embodiment 46. Use of the compound or pharmaceutically acceptable salt and/or tautomer thereof according to any one of Embodiments 1 to 39a in the treatment of cancer.

Embodiment 47. Use of the compound or pharmaceutically acceptable salt and/or tautomer thereof according to any one of Embodiments 1 to 39a in the manufacture of a medicament for the treatment of cancer.

Embodiment 48. The method according to Embodiment 43, the compound for use according to Embodiment 45, or the use according to Embodiment 46 or Embodiment 47, wherein the cancer is selected from ovarian cancer, gastric cancer, uterine cancer, breast cancer (e.g. ER+ breast cancer, e.g. ER+/Her2− breast cancer), lung cancer and endometrial cancer.

Embodiment 49. The method according to Embodiment 43 or Embodiment 48, the compound for use according to Embodiment 45 or Embodiment 48, or the use according to any one of Embodiments 46 to 48, wherein the cancer is a cyclin E amplified cancer.

Further Embodiments of the Invention are defined below in Embodiments A to LL:

Embodiment A. A compound according to formula (I),

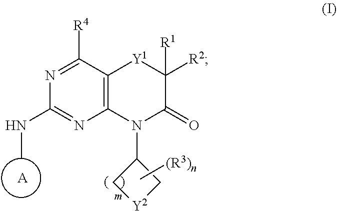

wherein:
Y¹ is a bond or CH₂ (e.g. Y¹ is a bond);
Y² is a bond, O, NR⁵ or CR⁶R⁷ (e.g. Y² is a bond);
R¹ and R² are each independently selected from the group consisting of H, halo, C₁-C₆alkyl and C₁-C₆haloalkyl, or R¹ and R² join together to form C₃-C₄cycloalkyl or C₃-C₄cyclohaloalkyl (e.g. R¹ and R² join together to form C₃-C₄cycloalkyl, e.g. R¹ and R² join together to form C₃cycloalkyl);
each R³ is independently selected from the group consisting of hydroxyl, halo, C₁-C₆alkyl and C₁-C₆haloalkyl;
R⁴ is selected from the group consisting of H, halo, C₁-C₆alkyl and C₁-C₆haloalkyl (e.g. R⁴ is H);
R⁵ is selected from the group consisting of H, C₁-C₆alkyl, C(═O)—C₁-C₆alkyl or C(═O)—O—C₁-C₆alkyl;
R⁶ and R⁷ join together to form, together with the carbon atom to which they are mutually attached, a C₃-C₆cycloalkyl or a 3-6 membered heterocyclyl comprising 1-3 heteroatoms independently selected from the group consisting of O, N and S, wherein said C₃-C₆cycloalkyl or 3-6 membered heterocyclyl is substituted with 0-3 substituents R⁸;
each R⁸ is independently selected from the group consisting of C₁-C₆alkyl, C(═O)C₁-C₆alkyl, halo, C₁-C₆haloalkyl, S—C₁-C₆alkyl, SO—C₁-C₆alkyl, SO₂—C₁-C₆alkyl, cyano, hydroxyl, or wherein two R⁸ substituents on the same ring atom join together to form ═O.
n is 0 to 3 (e.g. n is 1, e.g. n is 1 and R³ is hydroxyl);
m is 1 to 5 (e.g. m is 4);

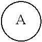

is a 5 membered heteroaryl selected from the group consisting of

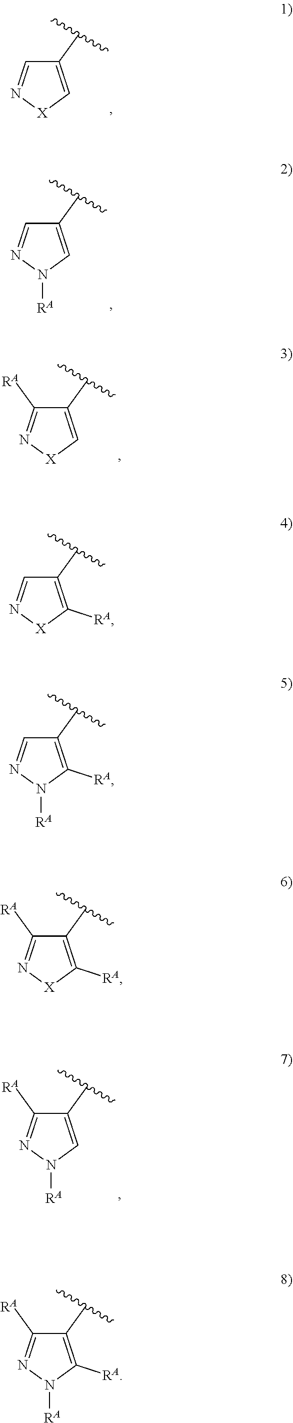

wherein X is selected from O, NH and S;
each R^{A} is independently *L¹-X¹, wherein * indicates the point of attachment to

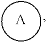

each L¹ is independently selected from bond, O, S, SO, SO₂, C≡C, C(═O), *C(═O)—O**, C₁-C₆alkylene, C₁-C₆haloalkylene, *O—C₁-C₆alkylene**, *O—C₁-C₆haloalkylene**, *O—C₁-C₆hydroxyalkylene**, C₁-C₆alkylene-O—C₁-C₆alkylene, *O—C₃-C₆cycloalkylene**, *O-3-6 membered heterocyclylene**, C₁-C₆hydroxyalkylene, C₃-C₆cycloalkylene, 3-6 membered heterocyclylene (e.g. comprising 1 heteroatom which is O), O—C₁-C₆alkylene-O, *O—C₁-C₆alkylene-O—C₃-C₆cycloalkylene** and *O-C₁-C₆alkylene-O-3-6 membered heterocyclylene**, wherein * indicates the point of attachment to

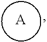

and ** indicates the point of attachment to X¹;
and each X¹ is independently selected from H, halo, cyano, hydroxyl, C₁-C₆alkyl, C(═O)—C₁-C₆alkyl, C₁-C₆haloalkyl, C₃-C₆cycloalkyl substituted by 0-3 R⁸ groups, O—C₁-C₆alkyl, S—C₁-C₆alkyl, S(O)—C₁-C₆alkyl, S(O)₂—C₁-C₆alkyl, N(C₁-C₆alkyl)₂, C(═O)N(C₁-C₆alkyl)₂, C₁-C₆hydroxyalkyl, 3-6 membered heterocyclyl (e.g. comprising 1 or 2 heteroatoms independently selected from O, N and S) substituted by 0-3 R⁸ groups, 5-10 membered heteroaryl (e.g. 5, 6, 9 or 10 membered heteroaryl) comprising 1-4 heteroatoms independently selected from O, N and S substituted by 0-3 R⁸ groups, 5-10 membered partially saturated heterocyclyl comprising 1 to 4 heteroatoms independently selected from the group consisting of O, N and S substituted by 0-3 R⁸ groups, 7-10 membered spiroheterocyclyl comprising 1 or 2 heteroatoms independently selected from O, N and S substituted by 0-3 R⁸ groups and C₇-C₁₀spirocycloalkyl substituted by 0-3 R⁸ groups;
or
two R^{A} substituents located on adjacent ring atoms join together to form with said adjacent ring atoms a 4 to 6 membered heterocyclyl comprising 1 to 3 heteroatoms independently selected from N, O and S, with the proviso that at least one heteroatom is N;
or a pharmaceutically acceptable salt and/or tautomer thereof.

Embodiment B. The compound or pharmaceutically acceptable salt and/or tautomer thereof according to Embodiment A, wherein Y¹ is a bond.

Embodiment C. The compound or pharmaceutically acceptable salt and/or tautomer thereof according to Embodiment A or Embodiment B, wherein Y² is a bond.

Embodiment D. The compound or pharmaceutically acceptable salt and/or tautomer thereof according to any one of Embodiments A to C, wherein m is 4.

Embodiment E. The compound or pharmaceutically acceptable salt and/or tautomer thereof according to any one of Embodiments A to D, wherein n is 1 to 3, e.g. n is 1.

Embodiment F. The compound or pharmaceutically acceptable salt and/or tautomer thereof according to Embodiment E, wherein at least one R³ is OH.

Embodiment G. The compound or pharmaceutically acceptable salt and/or tautomer thereof according to Embodiment A, wherein the compound of formula (I) is a compound of formula (Ia):

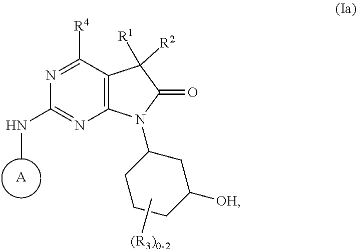

wherein R¹, R², R³, R⁴ and

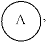

are as defined in Embodiment A.

Embodiment Ga. The compound or pharmaceutically acceptable salt and/or tautomer thereof according to Embodiment G, wherein the compound of formula (Ia) is a compound of formula (Ia-I):

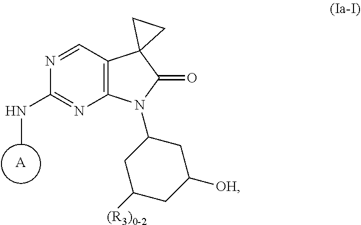

wherein R³ and

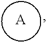

are as defined in Embodiment A.

Embodiment H. The compound or pharmaceutically acceptable salt and/or tautomer thereof according to Embodiment A, wherein the compound of formula (I) is a compound of formula (Ib):

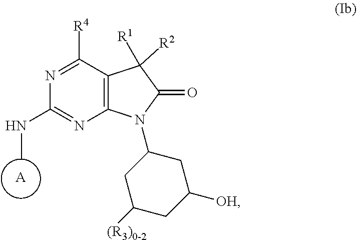

wherein R¹, R², R³, R⁴ and

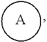

are as defined in Embodiment A.

Embodiment Ha. The compound or pharmaceutically acceptable salt and/or tautomer thereof according to Embodiment H, wherein the compound of formula (Ib) is a compound of formula (Ib-1):

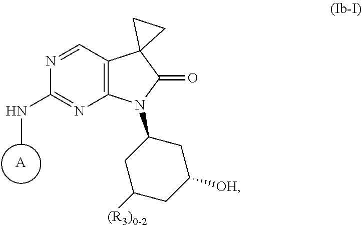

wherein R³ and

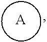

are as defined in Embodiment A.

Embodiment I. The compound or pharmaceutically acceptable salt and/or tautomer thereof according Embodiment A, wherein the compound of formula (I) is a compound of formula (Ic):

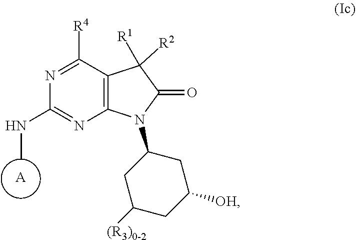

wherein R¹, R², R³, R⁴ and

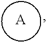

are as defined in Embodiment A.

Embodiment Ia. The compound or pharmaceutically acceptable salt and/or tautomer thereof according to Embodiment I, wherein the compound of formula (Ic) is a compound of formula (Ic-I):

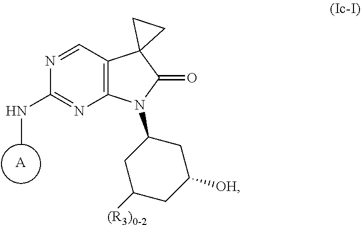

wherein R³ and

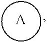

are as defined in Embodiment A.

Embodiment J. The compound or pharmaceutically acceptable salt and/or tautomer thereof according to Embodiment 1, wherein the compound of formula (I) is a compound of formula (Id):

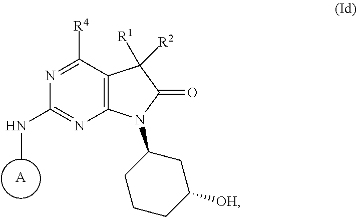

wherein R¹, R², R⁴ and

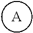

are as defined in Embodiment A.

Embodiment Ja. The compound or pharmaceutically acceptable salt and/or tautomer thereof according to Embodiment J, wherein the compound of formula (Id) is a compound of formula (Id-I):

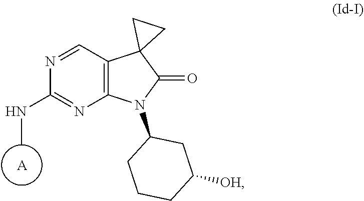

wherein

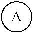

is as defined in Embodiment A.

Embodiment K. The compound or pharmaceutically acceptable salt and/or tautomer thereof according to any one of Embodiments A to Ja, wherein R¹ and R² join together to form C₃-C₄cycloalkyl or C₃-C₄cyclohaloalkyl.

Embodiment L. The compound or pharmaceutically acceptable salt and/or tautomer thereof according to Embodiment K, wherein R¹ and R² join together to form C₃-C₄cycloalkyl.

Embodiment M. The compound or pharmaceutically acceptable salt and/or tautomer thereof according to Embodiment L, wherein R¹ and R² join together to form C₃cycloalkyl.

Embodiment N. The compound or pharmaceutically acceptable salt and/or tautomer thereof according to any one of Embodiments A to M, wherein R⁴ is H.

Embodiment O. The compound or pharmaceutically acceptable salt and/or tautomer thereof according to any one of Embodiments A to N, wherein X is NH.

Embodiment P. The compound or pharmaceutically acceptable salt and/or tautomer thereof according to any one of Embodiments A to O, wherein

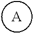

is selected from the group consisting of

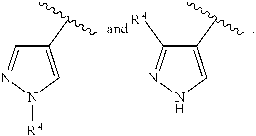

Embodiment Q. The compound or pharmaceutically acceptable salt and/or tautomer thereof according to Embodiment P, wherein

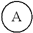

is

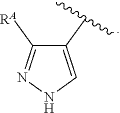

Embodiment R. The compound or pharmaceutically acceptable salt and/or tautomer thereof according to Embodiment Q, wherein R^{A} is selected from
a) halo,
b) cyano,
c) C₁-C₆alkyl,
d) C₁-C₆haloalkyl,
e) C₁-C₆hydroxyalkyl,
f) O—C₁-C₆alkyl,
g) C(═O)—O—C₁-C₆alkyl,
h) C₁-C₆alkylene-O—C₁-C₆alkyl,
i) O—C₁-C₆alkylene-O—C₁-C₆alkyl,
j) C₃-C₆cycloalkyl substituted by 0-3 R⁸ groups,
k) 5-10 membered heteroaryl comprising 1-4 heteroatoms independently selected from N, O and S substituted by 0-3 R⁸ groups,
l) 5-10 membered partially saturated heterocyclyl comprising 1-4 heteroatoms independently selected from N, O and S substituted by 0-3 R⁸ groups,
m) O—C₃-C₆cycloalkyl substituted by 0-3 R⁸ groups,
n) C≡C-5-10 membered heteroaryl comprising 1-4 heteroatoms independently selected from N, O and S substituted by 0-3 R⁸ groups,
o) S-5-10 membered heteroaryl comprising 1-4 heteroatoms independently selected from N, O and S substituted by 0-3 R⁸ groups,
p) C₁-C₆alkylene-5-10 membered heteroaryl comprising 1-4 heteroatoms independently selected from N, O and S substituted by 0-3 R⁸ groups,
q) O—C₁-C₆haloalkyl,
r) O—C₁-C₆alkylene-N(C₁-C₆alkyl)₂,
s) O—C₁-C₆hydroxyalkylene-O—C₁-C₆alkyl,
t) O—C₁-C₆alkylene-C₃-C₆cycloalkyl substituted by 0-3 R⁸ groups,
u) O—C₁-C₆alkylene-3-6 membered heterocyclyl comprising 1 or 2 heteroatoms independently selected from O, N and S substituted by 0-3 R⁸ groups,
v) O—C₁-C₆alkylene-C(═O)—N(C₁-C₆alkyl)₂,
w) C₁-C₆alkylene-5-10 membered partially saturated heterocyclyl comprising 1-4 heteroatoms independently selected from N, O and S substituted by 0-3 R⁸ groups,
x) O—C₁-C₆alkylene-7-10 membered spiroheterocyclyl comprising 1 or 2 heteroatoms independently selected from O, N and S substituted by 0-3 R⁸ groups,
y) O—C₁-C₆alkylene-S(O)₂—C₁-C₆alkyl,
z) O—C₁-C₆hydroxyalkyl,
aa) O—C₁-C₆alkylene-5-10 membered heteroaryl comprising 1-4 heteroatoms independently selected from N, O and S substituted by 0-3 R⁸ groups,
bb) O-5-10 membered heteroaryl comprising 1-4 heteroatoms independently selected from N, O and S substituted by 0-3 R⁸ groups,
cc) O-3-6 membered heterocyclyl comprising 1 or 2 heteroatoms independently selected from O, N and S substituted by 0-3 R⁸ groups,
dd) C≡C—C₃-C₆cycloalkyl substituted by 0-3 R⁸ groups,
ee) S—C₁-C₆haloalkyl,
ff) O—C₁-C₆alkylene-O-3-6 membered heterocyclyl comprising 1 or 2 heteroatoms independently selected from O, N and S substituted by 0-3 R⁸ groups
gg) and 3 to 6 membered heterocyclyl comprising 1 or 2 heteroatoms selected from N, O and S substituted by 0-3 R⁸ groups.

Embodiment 5. The compound or pharmaceutically acceptable salt and/or tautomer thereof according to Embodiment R, wherein each R⁸ is independently selected from the group consisting of halo (e.g. fluoro), C₁-C₆alkyl (e.g. methyl), hydroxyl, cyano, S(O₂)—C₁-C₆alkyl (e.g. S(O₂)CH₃), C(═O)—C₁-C₆alkyl (e.g. C(═O)CH₃), O—C₁-C₆alkyl (e.g. OCH₃) and C₁-C₆haloalkyl (e.g. C₁haloalkyl, e.g. CHF₂), or wherein two R⁸ substituents on the same ring atom join together to form ═O.

Embodiment T. The compound or pharmaceutically acceptable salt and/or tautomer thereof according to any one of Embodiments A to S, wherein

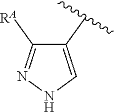

and R^{A} is CH₃, OCH₃ or OCH₂CH₃.

Embodiment U. The compound or pharmaceutically acceptable salt and/or tautomer thereof according to any one of Embodiments A to R, wherein each L¹ is independently selected from bond, O, C(═O), *C(═O)—O**, C₁-C₆alkylene, C₁-C₆haloalkylene, *O—C₁-C₆alkylene**, C₁-C₆alkylene-O—C₁-C₆alkylene, C₁-C₆hydroxyalkylene, C₃-C₆cycloalkylene, 3-6 membered heterocyclylene (e.g. comprising 1 heteroatom which is O) and O—C₁-C₆alkylene-O, wherein * indicates the point of attachment to

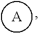

and ** indicates the point of attachment to X¹;
and each X¹ is independently selected from H, halo, cyano, hydroxyl, C₁-C₆alkyl, C₁-C₆haloalkyl, C₃-C₆cycloalkyl, O—C₁-C₆alkyl, C₁-C₆hydroxyalkyl and 3-6 membered heterocyclyl (e.g. comprising 1 heteroatom that is O).

Embodiment Ua. The compound or pharmaceutically acceptable salt and/or tautomer thereof according to Embodiment U, wherein each L¹ is independently selected from bond, O, C(═O), *C(═O)—O**, C₁-C₂alkylene, C₁haloalkylene, *O—C₁-C₂alkylene**, C₁-C₂alkylene-O—C₁alkylene, C₁-hydroxyalkylene, C₃cycloalkylene, 5-6 membered heterocyclylene comprising 1 heteroatom which is O and O—C₂alkylene-O, wherein * indicates the point of attachment to

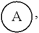

and ** indicates the point of attachment to X¹.

Embodiment Ub. The compound or pharmaceutically acceptable salt and/or tautomer thereof according to Embodiment U or Embodiment Ua, wherein each X¹ is independently selected from H, halo, cyano, hydroxyl, C₁-C₂alkyl (e.g. C₁alkyl), C₁haloalkyl, C₃cycloalkyl, O—C₁-C₂alkyl, C₁hydroxyalkyl and 5-6 membered heterocyclyl comprising 1 heteroatom that is O.

Embodiment V. The compound or pharmaceutically acceptable salt and/or tautomer thereof according to any one of Embodiments A to R or Embodiment U, wherein R^{A} is selected from the list consisting of C₁-C₆alkyl, C₁-C₆alkylene-O—C₁-C₆alkyl, C₁-C₆haloalkyl, C(═O)—O—C₁-C₆alkyl, C₁-C₆hydroxyalkyl, 3-6 membered heteroatom comprising 1 heteroatom that is O, halo, O—C₁-C₆alkyl, C₃-C₆cycloalkyl, cyano and O—C₁-C₆alkylene-O—C₁-C₆alkyl.

Embodiment Va. The compound or pharmaceutically acceptable salt and/or tautomer thereof according to Embodiment V, wherein R^{A} is selected from the list consisting of CH₃, OCH₂CH₃, CH₂CH₂OCH₃, CHF₂, C(═O)—O—CH₃, CH₂OH, 5 membered heteroatom comprising 1 heteroatom that is O, 6 membered heteroatom comprising 1 heteroatom that is O, CH₂OCH₃, OCH₃, Cl, C₃cycloalkyl, CF₃, cyano, OCH₂CH₂OCH₃ and OCH₂CH₃.

Embodiment W. A compound selected from any one of:
1) 7′-((1R,3R)-3-hydroxycyclohexyl)-2′-((3-methyl-1H-pyrazol-4-yl)amino)spiro[cyclopropane-1,5′-pyrrolo[2,3-d]pyrimidin]-6′(7′H)-one;
2) 7′-((1R,3R)-3-hydroxycyclohexyl)-2′-((1-(2-methoxyethyl)-1H-pyrazol-4-yl)amino)spiro[cyclopropane-1,5′-pyrrolo[2,3-d]pyrimidin]-6′(7′H)-one;
3) 2′-((3-(difluoromethyl)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
4) methyl 4-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)-1H-pyrazole-3-carboxylate;
5) 7′-((1R,3R)-3-hydroxycyclohexyl)-2′-((3-(hydroxymethyl)-1H-pyrazol-4-yl)amino)spiro[cyclopropane-1,5′-pyrrolo[2,3-d]pyrimidin]-6′(7′H)-one;
6) 7′-((1R,5R)-5-hydroxy-3,3-dimethylcyclohexyl)-2′-((3-methyl-1H-pyrazol-4-yl)amino)spiro[cyclopropane-1,5′-pyrrolo[2,3-d]pyrimidin]-6′(7′H)-one;
7) 7′-((1R,3R)-3-hydroxycyclohexyl)-2′-((3-(tetrahydrofuran-3-yl)-1H-pyrazol-4-yl)amino)spiro[cyclopropane-1,5′-pyrrolo[2,3-d]pyrimidin]-6′(7′H)-one;
8) 7′-((1R,3R)-3-hydroxycyclohexyl)-2′-((4,5,6,7-tetrahydropyrazolo[1,5-a]pyrazin-3-yl)amino)spiro[cyclopropane-1,5′-pyrrolo[2,3-d]pyrimidin]-6′(7′H)-one;
9) 7′-((1R,3R)-3-hydroxycyclohexyl)-2′-((3-(tetrahydro-2H-pyran-2-yl)-1H-pyrazol-4-yl)amino)spiro[cyclopropane-1,5′-pyrrolo[2,3-d]pyrimidin]-6′(7′H)-one;
10) 7′-((1R,3R)-3-hydroxycyclohexyl)-2′-((3-methoxy-1H-pyrazol-4-yl)amino)spiro[cyclopropane-1,5′-pyrrolo[2,3-d]pyrimidin]-6′(7′H)-one;
11) 2′-((3-chloro-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
12) 2′-((3-cyclopropyl-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
13) 7′-((1R,3R)-3-hydroxycyclohexyl)-2′-((3-(trifluoromethyl)-1H-pyrazol-4-yl)amino)spiro[cyclopropane-1,5′-pyrrolo[2,3-d]pyrimidin]-6′(7′H)-one;
14) 4-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)-1H-pyrazole-3-carbonitrile;
15) 7′-((1R,3R)-3-hydroxycyclohexyl)-2′-((3-(methoxymethyl)-1H-pyrazol-4-yl)amino)spiro[cyclopropane-1,5′-pyrrolo[2,3-d]pyrimidin]-6′(7′H)-one;
16) 7′-((1R,3R)-3-hydroxycycloheptyl)-2′-((3-methyl-1H-pyrazol-4-yl)amino)spiro[cyclopropane-1,5′-pyrrolo[2,3-d]pyrimidin]-6′(7′H)-one;
17) 7′-((1R,3R)-3-hydroxycycloheptyl)-2′-((3-methoxy-1H-pyrazol-4-yl)amino)spiro[cyclopropane-1,5′-pyrrolo[2,3-d]pyrimidin]-6′(7′H)-one;
18) 2′-((3-chloro-1H-pyrazol-4-yl)amino)-7′-((1R,3R)-3-hydroxycycloheptyl)spiro[cyclopropane-1,5′-pyrrolo[2,3-d]pyrimidin]-6′(7′H)-one;
19) 7′-((1R,3R)-3-hydroxycyclohexyl)-2′-((3-(2-methoxyethoxy)-1H-pyrazol-4-yl)amino)spiro[cyclopropane-1,5′-pyrrolo[2,3-d]pyrimidin]-6′(7′H)-one; and
20) 2′-((3-ethoxy-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;

or a pharmaceutically acceptable salt and/or tautomer thereof.

Embodiment X. The compound according to Embodiment A, wherein the compound is

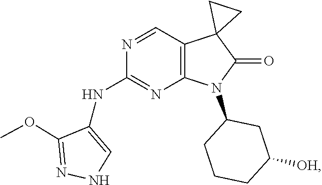 [SMILES: COc1n[nH]cc1Nc1ncc2c(n1)N([C@@H]1CCC[C@@H](O)C1)C(=O)C21CC1]

or a pharmaceutically acceptable salt and/or tautomer thereof.

Embodiment Y. The compound according to Embodiment A, wherein the compound is

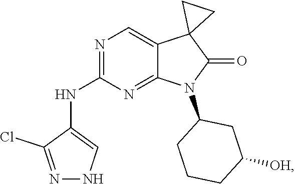 [SMILES: O=C1N([C@@H]2CCC[C@@H](O)C2)c2nc(Nc3c[nH]nc3Cl)ncc2C12CC2]

or a pharmaceutically acceptable salt and/or tautomer thereof.

Embodiment Z. The compound according to Embodiment A, wherein the compound is

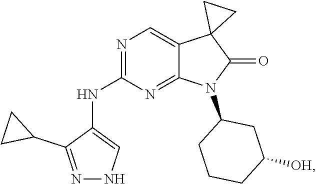 [SMILES: O=C1N([C@@H]2CCC[C@@H](O)C2)c2nc(Nc3c[nH]nc3C3CC3)ncc2C12CC2]

or a pharmaceutically acceptable salt and/or tautomer thereof.

Embodiment AA. The compound according to Embodiment A, wherein the compound is

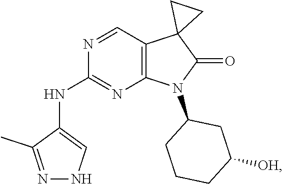 [SMILES: Cc1n[nH]cc1Nc1ncc2c(n1)N([C@@H]1CCC[C@@H](O)C1)C(=O)C21CC1]

or a pharmaceutically acceptable salt and/or tautomer thereof.

Embodiment BB. The compound according to Embodiment A, wherein the compound is

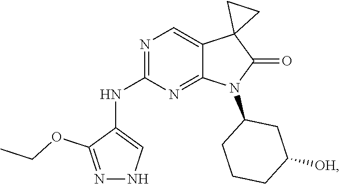 [SMILES: CCOc1n[nH]cc1Nc1ncc2c(n1)N([C@@H]1CCC[C@@H](O)C1)C(=O)C21CC1]

or a pharmaceutically acceptable salt and/or tautomer thereof.

Embodiment CC. A pharmaceutical composition comprising the compound or pharmaceutically acceptable salt and/or tautomer thereof according to any one of Embodiments A to BB, and one or more pharmaceutically acceptable carriers.

Embodiment DD. A combination comprising the compound or pharmaceutically acceptable salt and/or tautomer thereof according to any one of Embodiments A to BB, and one or more therapeutically active agents.

Embodiment EE. A method of modulating CDK2 activity in a subject comprising administering to the subject a therapeutically effective amount of the compound or pharmaceutically acceptable salt and/or tautomer thereof according to any one of Embodiments A to BB.

Embodiment FF. A method of treating cancer comprising administering to a subject in need thereof a therapeutically effective amount of the compound or pharmaceutically acceptable salt and/or tautomer thereof according to any one of Embodiments A to BB.

Embodiment GG. A compound or pharmaceutically acceptable salt and/or tautomer thereof according to any one of Embodiments A to BB for use as a medicament.

Embodiment HH. A compound or pharmaceutically acceptable salt and/or tautomer thereof according to any one of Embodiments A to BB for use in the treatment of cancer.

Embodiment II. Use of the compound or pharmaceutically acceptable salt and/or tautomer thereof according to any one of Embodiments A to BB in the treatment of cancer.

Embodiment JJ. Use of the compound or pharmaceutically acceptable salt and/or tautomer thereof according to any one of Embodiments A to BB in the manufacture of a medicament for the treatment of cancer.

Embodiment KK. The method according to Embodiment FF, the compound for use according to Embodiment HH, or the use according to Embodiment II or Embodiment JJ, wherein the cancer is selected from ovarian cancer, gastric cancer, uterine cancer, breast cancer (e.g. ER+ breast cancer, e.g. ER+/Her2− breast cancer), lung cancer and endometrial cancer.

Embodiment LL. The method according to Embodiment FF or Embodiment KK, the compound for use according to Embodiment HH or Embodiment KK, or the use according to any one of Embodiments II to KK, wherein the cancer is a cyclin E amplified cancer.

### Definitions

For the purpose of interpreting this specification, the following definitions will apply unless specified otherwise and when appropriate, terms used in the singular will also include the plural and vice versa. It must be noted that as used herein and in the appended claims, the singular forms “a”, “an” and “the” include the plural unless the context clearly dictates otherwise. Thus, for example, reference to “the compound” includes reference to one or more compounds, and so forth.

As used herein, the term “substituent” refers to a radical group which replaces a hydrogen atom in a given molecule. In groups such as

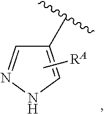

a hydrogen atom is (necessarily) shown explicitly in the structure on a heteroatom. The substituent (in this case, R^{A}) can be replace any hydrogen atom, including that explicitly shown. In other words,

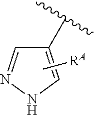

### can be H

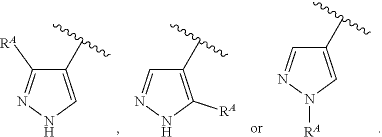

As used herein, the term “alkyl” refers to a straight or branched hydrocarbon chain radical consisting solely of carbon and hydrogen atoms, containing no unsaturation, and which is attached to the rest of the molecule by a single bond. For instance, C₁-C₆alkyl contains from 1 to 6 carbon atoms. Examples of C₁-C₆alkyl include, but are not limited to, methyl (Me), ethyl (Et), n-propyl, 1-methylethyl (iso-propyl), n-butyl, t-butyl, pentyl, isopentyl, neopentyl, hexyl, 2-methylpentyl, 3-methylpentyl, 2,3-dimethylbutyl and 2,2-dimethylbutyl.

As used herein, the term “halogen”, “halo”, “hal”, etc. refers to fluorine, chlorine, bromine or iodine. Halogen-substituted groups and moieties, such as alkyl substituted by halogen (haloalkyl) can be mono-, poly- or per-halogenated.

As used herein, the term “haloalkyl” refers to an alkyl radical as defined herein, wherein one or more of the hydrogen atoms of said alkyl has been replaced with a halogen atom. Particularly said one or more halogen atom(s) are each fluorine atom(s), in which case the “haloalkyl” is a “fluoroalkyl”. For instance, C₁-C₆haloalkyl contains from 1 to 6 carbon atoms (and 1 or more halogen atoms).

As used herein, the term “hydroxyalkyl” refers to an alkyl radical as defined herein, wherein one or more of the hydrogen atoms of said alkyl has been replaced with a hydroxyl group. For instance, C₁-C₆hydroxyalkyl contains from 1 to 6 carbon atoms (and 1 or more hydroxyl groups).

As used herein, the term “alkylene” refers to a straight-chain or branched divalent radical of an alkyl group. For instance, “C₁-C₄alkylene” contains from 1 to 4 carbon atoms e.g., —CH₂—, —CH₂CH₂—, —CH₂CH₂CH₂—, —CH(CH₃)₂—, —CH₂CH(CH₃)CH₂—.

Likewise, as used herein, the term “haloalkylene” refers to a straight-chain or branched divalent radical of a haloalkyl group.

As used herein, the term “cycloalkyl” refers to a saturated carbocyclic ring radical. C₃-C₆cycloalkyl for instance, is any such ring radical containing 3 to 6 carbon atoms, and is particularly monocyclic i.e. cyclobutyl, cyclopentyl and cyclohexyl. However, the cycloalkyl (e.g. C₃-C₆cycloalkyl) can also be a fused

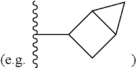 [SMILES: *C1CC2CC12]

or bridged

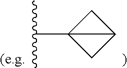 [SMILES: *C12CC1C2]

bicyclic ring system.

The term “cycloalkylene” refers to a divalent radical of a cycloalkyl group.

As used herein, the term “cyclohaloalkyl” refers to a cycloalkyl radical as defined herein, wherein one or more of the hydrogen atoms of said cycloalkyl has been replaced with a halogen atom. Particularly said one or more halogen atom(s) are each fluorine atom(s), in which case the “cyclohaloalkyl” is a “cyclofluoroalkyl”. As with cycloalkyls, a cyclohaloalkyl can be a fused or bridged bicyclic ring system.

As used herein, the term “heterocyclyl”, “heterocycle”, “heterocyclic” etc. refers to a heterocyclic radical that is saturated or partially unsaturated but not aromatic, and can be a monocyclic or a polycyclic ring, including a fused or bridged bicyclic ring system. Particularly, however, the heterocyclyl is a monocyclic ring. A heterocyclyl contains at least one non-carbon atom as a ring member, typically nitrogen, oxygen or sulfur unless otherwise specified, the remaining ring atoms therefore being carbon. Preferably the number of heteroatoms in the heterocyclyl is from 1 to 3, wherein each heteroatom is independently selected from nitrogen, oxygen and sulfur. Where a heterocyclyl contains S as a heteroatom, the S can be in the form of S, SO or SO₂ (in other words, the oxygen atoms bonded to the sulphur do not constitute substitutions). For example, the term “4-6 membered heterocyclyl comprising 1 heteroatom selected from the group consisting of O, N and S” refers to a ring radical containing 4 to 6 ring atoms comprising 1 heteroatom (either O, N, or S [the latter including S, SO and SO₂]), with the remaining ring atoms being carbon.

The term “heterocyclylene” refers to a divalent radical of a heterocyclyl group.

As used herein, the term “O-alkyl” refers to an alkyl radical as defined herein, which is attached to the rest of the molecule via an O linker. An example is OCH₃.

As used herein, the term “O-haloalkyl” refers to a haloalkyl radical as defined herein, which is attached to the rest of the molecule via an O linker. An example is OCF₃.

As used herein, the term “alkylene-O-alkyl” refers to an alkyl radical as defined herein, wherein one of the hydrogen atoms of said alkyl radical has been replaced with —O-alkylene- (attached to the alkyl radical through the oxygen). An example is “C₁alkylene-O—C₁-alkyl”, i.e. —CH₂—O—CH₃.

The term “5-10 membered heteroaryl” is a monocyclic or bicyclic aromatic ring radical containing 5 to 10 ring atoms (e.g. 9 or 10 ring atoms in a bicyclic aromatic ring radical) which, unless otherwise stated, comprises 1, 2, 3 or 4 heteroatoms independently selected from nitrogen, oxygen and sulfur in the ring radical.

The term “5 membered heteroaryl” is a monocyclic aromatic ring radical which, unless otherwise stated, comprises 1, 2 or 3 heteroatoms (preferably 2) individually selected from nitrogen, oxygen and sulfur (which can be in the form of S, SO or SO₂, particularly S) in the ring radical. Preferably, at least one of the heteroatoms in the 5 membered heteroaryl is nitrogen. Preferably, there are two heteroatoms in the 5 membered heteroaryl, at least one of which is nitrogen. Most preferably, there are two heteroatoms in the 5 membered heteroaryl, both of which are nitrogen.

As used herein, the term “partially saturated heterocyclyl” is intended to include partially saturated monocyclic, bicyclic or polycyclic heterocyclyls containing one or more heteroatoms selected 0, N, and S (S where present may be in the form of S, SO or SO₂). Representative examples are imidazolinyl, indolinyl, dihydrobenzofuranyl, dihydrobenzothienyl, dihydrobenzopyranyl, dihydropyridooxazinyl, dihydrobenzodioxinyl (e.g., 2,3-dihydrobenzo[b][1,4]dioxinyl), benzodioxolyl (e.g., benzo[d][1,3]dioxole), dihydrobenzooxazinyl (e.g., 3,4-dihydro-2H-benzo[b][1,4]oxazine), tetrahydroindazolyl, tetrahydrobenzimidazolyl, tetrahydroimidazo[4,5-c]pyridyl, tetrahydroquinolinyl, tetrahydroisoquinolinyl, tetrahydroquinoxalinyl, and the like. The term “5- to 10-membered partially saturated heterocyclyl” is to be construed accordingly.

As used herein, the term “spiroheterocyclyl”, refers to ring system comprising a first carbocyclic or heterocyclic ring comprising from 3 to 6 ring atoms wherein two of the substituents on a carbon ring atom in said first carbocyclic or heterocyclic ring join together to form a second carbocyclic or heterocyclic ring comprising from 3 to 6 ring atoms, with the proviso that at least one of the first and second rings is a heterocyclic ring comprising one or more heteroatoms selected from the group consisting of O, N and S (the latter can be in the form of S, SO or SO₂), particularly selected from the group consisting of 0 and N. Particularly, the spiroheterocyclyl is saturated. The term 7-9 membered spiroheterocyclyl, as used herein means that the total number of ring atoms in the first carbocyclic or heterocyclic ring and the second carbocyclic or heterocyclic ring is from 7 to 9.

For instance, the spiroheterocyclyl

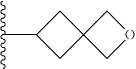 [SMILES: *C1CC2(COC2)C1]

is a 7 membered spiroheterocyclyl, as there are 7 ring atoms present. As will be appreciated by the skilled person, a “spiroheterocyclyl” is a mono-radical, whereas a “spiroheterocyclylene” is a di-radical (analogous to alkyl and alkylene).

As used herein, the term “spirocycloalkyl” refers to a ring system comprising a first carbocyclic ring comprising from 3 to 6 ring carbon atoms, wherein two of the substituents on a carbon ring atom in said first carbocyclic ring join together to form a second carbocyclic ring comprising from 3 to 6 ring carbon atoms. Particularly, the spirocycloalkyl is saturated. The term 6-8 membered spirocycloalkyl, as used herein means that the total number of carbon ring atoms in the first carbocyclic ring and the second carbocyclic ring is from 7 to 8. As will be appreciated by the skilled person a “spirocycloalkylene” is a di-radical equivalent to a “spirocycloalkyl”.

The term “two R^{A} substituents located on adjacent ring atoms join together to form with said adjacent ring atoms a 4 to 6 membered heterocyclyl” as used herein refers to (taking the example of

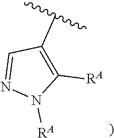

heterocycles such as

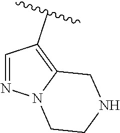 [SMILES: *c1cnn2c1CNCC2]

in this example, a 6 membered heterocycle containing 2 heteroatoms which are both N).

Depending on the choice of the starting materials and procedures, the compounds can be present in the form of one of the possible stereoisomers or as mixtures thereof, for example as pure optical isomers, or as stereoisomer mixtures, such as racemates and diastereoisomer mixtures, depending on the number of asymmetric carbon atoms. The present invention is meant to include all such possible stereoisomers, including racemic mixtures, diasteriomeric mixtures and optically pure forms. Optically active (R)- and (S)-stereoisomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included. Many compounds of the invention exhibit tautomerism. For instance,

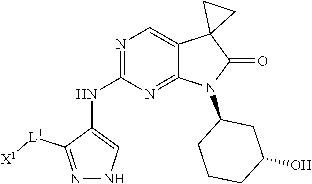

can tautomerize to

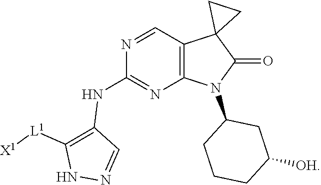

All tautomeric forms are within the scope of the claimed invention.

As used herein, the terms “salt” or “salts” refers to an acid addition or base addition salt of a compound of the present invention. “Salts” include in particular “pharmaceutical acceptable salts”.

The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto. When both a basic group and an acid group are present in the same molecule, the compounds of the present invention may also form internal salts, e.g., zwitterionic molecules.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper;
particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In another aspect, the present invention provides compounds of the present invention in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form.

In another aspect, the present invention provides compounds of the present invention in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form.

In another aspect, the present invention provides compounds according to any one of embodiments 1 to 39a, in sodium, potassium, ammonium, calcium, magnesium, iron, silver, zinc, copper, isopropylamine, benzathine, cholinate, diethanolamine, diethylamine, lysine, meglumine, piperazine or tromethamine salt form.

Any formula given herein is also intended to represent unlabelled forms as well as isotopically labelled forms of the compounds. Isotopically labelled compounds have structures depicted by the formulae given herein except that one or more atoms are replaced by an atom having a selected atomic mass or mass number. Isotopes that can be incorporated into compounds of the invention include, for example, isotopes of hydrogen.

Further, incorporation of certain isotopes, particularly deuterium (i.e., ²H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of a compound of the present invention. The concentration of deuterium, may be defined by the isotopic enrichment factor. The term “isotopic enrichment factor” as used herein means the ratio between the isotopic abundance and the natural abundance of a specified isotope. If a substituent in a compound of this invention is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into compounds of the invention include isotopes of hydrogen, carbon, nitrogen, oxygen, phosphorous, fluorine, and chlorine, such as ³H, ¹¹C, ¹³C, ¹⁴C, ¹⁵N, ¹⁸F, ³¹P, ³²P, ³⁵S, ³⁶Cl, ¹²³I, ¹²⁴I, ¹²⁵I respectively. Accordingly it should be understood that the invention includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of the present invention can generally be prepared by conventional techniques known to those skilled in the art or by processes analogous to those described in the accompanying Examples and Preparations using an appropriate isotopically-labeled reagenta in place of the non-labeled reagent previously employed.

### Pharmaceutical Composition

As used herein, the term “pharmaceutical composition” refers to a compound of the invention, or a pharmaceutically acceptable salt and/or tautomer thereof, together with at least one pharmaceutically acceptable carrier, in a form suitable for oral or parenteral administration.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22^{nd} Ed. Pharmaceutical Press, 2013, pp. 1049-1070).

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviate, inhibit, prevent and/or ameliorate a condition, or a disorder or a disease (i) mediated by CDK2, or (ii) associated with CDK2 activity, or (iii) characterized by activity (normal or abnormal) of CDK2; or (2) reduce or inhibit the activity of CDK2; or (3) reduce or inhibit the expression of CDK2. In another non-limiting embodiment, the term “a therapeutically effective amount” refers to the amount of the compound of the present invention that, when administered to a cell, or a tissue, or a non-cellular biological material, or a medium, is effective in at least partially reducing or inhibiting the activity of CDK2; or at least partially reducing or inhibiting the expression of CDK2.

As used herein, the term “subject” refers to primates (e.g., humans, male or female), dogs, rabbits, guinea pigs, pigs, rats and mice. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent”, “preventing” or “prevention” of any disease or disorder refers to the prophylactic treatment of the disease or disorder; or delaying the onset or progression of the disease or disorder.

As used herein, a subject is “in need of” a treatment if such subject would benefit biologically, medically, or in quality of life from such treatment.

As used herein, the term “a”, “an”, “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible stereoisomers, rotamers, atropisomers, tautomers or mixtures thereof, for example, as substantially pure geometric (cis or trans) stereoisomers, diastereomers, optical isomers (antipodes), racemates or mixtures thereof.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of compounds of the present invention or of intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid.

Racemic compounds of the present invention or racemic intermediates can also be resolved by chiral chromatography, e.g., high pressure liquid chromatography (HPLC) using a chiral adsorbent.

All methods described herein can be performed in any suitable order unless otherwise indicated herein or otherwise clearly contradicted by context. The use of any and all examples, or exemplary language (e.g. “such as”) provided herein is intended merely to better illustrate the invention and does not pose a limitation on the scope of the invention otherwise claimed.

The compounds of the present application can be prepared by those skilled in the art of organic synthesis using commercially available starting materials, compounds known in the literature, or from readily prepared intermediates, by employing standard synthetic methods and procedures either known to those skilled in the art, or which will be apparent to the skilled chemist in light of the teachings herein.

The compounds of Formula (I) may be prepared by methods as set forth in the following synthetic reaction schemes. In the schemes described below, it is well understood that protecting groups for sensitive or reactive groups are employed where necessary in accordance with general principles of chemistry. Protecting groups are manipulated according to standard methods of organic synthesis as described for example in Protective Groups in Organic Synthesis, 3rd edition, John Wiley & Sons: New York, 1999 or Protecting Groups, 3rd edition, Thieme, Stuttgart, 2004. Protective groups are removed at a convenient stage of the compound synthesis using methods that are readily apparent to those skilled in the art.

Those skilled in the art will recognize if a stereocentre exists in the compounds disclosed herein. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

Compounds of the present disclosure can be synthesized by following the steps outlined in Scheme 1. Starting materials are either commercially available or made by known procedures in the reported literature or as illustrated.

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure material.

Compounds of the invention and intermediates can also be converted into each other according to methods generally known to those skilled in the art.

In another aspect, the present invention provides a pharmaceutical composition comprising a compound of the present invention, or a pharmaceutically acceptable salt and/or tautomer thereof, and a pharmaceutically acceptable carrier. In a further embodiment, the composition comprises at least two pharmaceutically acceptable carriers, such as those described herein. The pharmaceutical composition can be formulated for particular routes of administration such as oral administration, parenteral administration (e.g. by injection, infusion, transdermal or topical administration), and rectal administration. Topical administration may also pertain to inhalation or intranasal application. The pharmaceutical compositions of the present invention can be made up in a solid form (including, without limitation, capsules, tablets, pills, granules, powders or suppositories), or in a liquid form (including, without limitation, solutions, suspensions or emulsions). Tablets may be either film coated or enteric coated according to methods known in the art. Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and
e) absorbents, colorants, flavors and sweeteners.

### METHODS OF USE OF THE INVENTION

The compounds of formula (I), in free form or in pharmaceutically acceptable salt form and/or tautomeric form, exhibit valuable pharmacological properties, for example CDK2 modulating properties, for example as indicated in in vitro tests as provided in the next sections, and are therefore indicated for therapy or for use as research chemicals, e.g. as tool compounds.

Compounds of the invention may be useful in the treatment, or prevention of cancer. In an embodiment, the cancer is selected from ovarian cancer, gastric cancer, uterine cancer, breast cancer (e.g. ER+ breast cancer, e.g. ER+/Her2− breast cancer), lung cancer and endometrial cancer.

Thus, as a further aspect, the present invention provides the use of a compound of formula (I), (in particular according to any one of embodiments 1 to 39a), or a pharmaceutically acceptable salt and/or tautomer thereof, in therapy. In a further embodiment, the therapy is treatment of a disease, disorder or condition which may be treated by inhibition of CDK2. In another embodiment, the cancer is selected from ovarian cancer, gastric cancer, uterine cancer, breast cancer (e.g. ER+ breast cancer, e.g. ER+/Her2− breast cancer), lung cancer and endometrial cancer.

Thus, as a further aspect, the present invention provides a compound of formula (I), (in particular according to any one of embodiments 1 to 39a), or a pharmaceutically acceptable salt and/or tautomer thereof, for use in therapy. In a further embodiment, the therapy is selected from a disease which may be treated by inhibition of CDK2. In another embodiment, the cancer is selected from ovarian cancer, gastric cancer, uterine cancer, breast cancer (e.g. ER+ breast cancer, e.g. ER+/Her2− breast cancer), lung cancer and endometrial cancer.

In another aspect, the invention provides a method of treating, or preventing a disease which is treated by inhibiting CDK2 comprising administration of a therapeutically effective amount of a compound of any one of formula (I) (in particular according to any one of embodiments 1 to 39a), or a pharmaceutically acceptable salt and/or tautomer thereof. In a further embodiment, the cancer is selected from ovarian cancer, gastric cancer, uterine cancer, breast cancer (e.g. ER+ breast cancer, e.g. ER+/Her2− breast cancer), lung cancer and endometrial cancer.

Thus, as a further aspect, the present invention provides the use of a compound of any one of formula (I) (in particular according to any one of embodiments 1 to 39a), or a pharmaceutically acceptable salt and/or tautomer thereof, for the manufacture of a medicament. In a further embodiment, the medicament is for treatment, or prevention of a disease, which may be treated by inhibition of CDK2. In another embodiment, the cancer is selected from ovarian cancer, gastric cancer, uterine cancer, breast cancer (e.g. ER+ breast cancer, e.g. ER+/Her2− breast cancer), lung cancer and endometrial cancer.

The pharmaceutical composition or combination of the present invention may, for example, be in unit dosage of about 1-1000 mg of active ingredient(s) for a subject of about 50-70 kg. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

### Combination Product and Combination Therapy of the Invention

“Combination” refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals, especially where these time intervals allow for the combination partners to have a cooperative, e.g. synergistic effect. The single components may be packaged in a kit or separately. One or both of the components (e.g. powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g. a compound of the present invention and a combination partner, are both administered to a patient simultaneously in the form of a single entity or dosage. The term “non-fixed combination” means that the therapeutic agents, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agents.

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow for the combination partners to have a cooperative, e.g. synergistic effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g. tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

The compounds of the present invention may be administered either simultaneously with, or before, or after, one or more other therapeutic agent. The compounds of the present invention may be administered separately, by the same or different route of administration, or together in the same pharmaceutical composition as the other agents. A therapeutic agent is, for example, a chemical compound, peptide, antibody, antibody fragment or nucleic acid, which is therapeutically active or enhances the therapeutic activity when administered to a patient in combination with a compound of the invention.

Thus, in another aspect, the invention provides a combination, in particular a pharmaceutical combination, comprising (e.g. a therapeutically effective amount of) a compound of formula (I) in particular according to any one of embodiments 1 to 39a), or a pharmaceutically acceptable salt and/or tautomer thereof, and one or more other therapeutically active agents.

In one embodiment, the invention provides a product comprising a compound of formula (I) in particular according to any one of embodiments 1 to 39a), or a pharmaceutically acceptable salt and/or tautomer thereof, and at least one other therapeutic agent as a combined preparation for simultaneous, separate or sequential use in therapy.

In one embodiment, the therapy is the treatment, or prevention of a disease or condition mediated by CDK2. Products provided as a combined preparation include a composition comprising a compound of formula (I) a pharmaceutically acceptable salt and/or tautomer thereof, and the other therapeutic agent(s) together in the same pharmaceutical composition, or a compound of formula (I) or a pharmaceutically acceptable salt and/or tautomer thereof, and the other therapeutic agent(s) in separate form, e.g. in the form of a kit.

In one embodiment, the invention provides a pharmaceutical combination comprising a compound of formula (I) (in particular according to any one of embodiments 1 to 39a), or a pharmaceutically acceptable salt and/or tautomer thereof, and another therapeutic agent(s). Optionally, the pharmaceutical combination may comprise a pharmaceutically acceptable carrier, as described above.

In one embodiment, the invention provides a kit comprising two or more separate pharmaceutical compositions, at least one of which contains a compound of formula (I) (in particular according to any one of embodiments 1 to 39a), or a pharmaceutically acceptable salt and/or tautomer thereof.

In one embodiment, the kit comprises means for separately retaining said compositions, such as a container, divided bottle, or divided foil packet. An example of such a kit is a blister pack, as typically used for the packaging of tablets, capsules and the like.

### EXAMPLES

The disclosure is further illustrated by the following examples and synthetic methods, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

The compounds of the present invention can be produced by organic synthesis methods known to one of ordinary skill in the art as shown in the following examples. All starting materials, building blocks, reagents, acids, bases, dehydrating agents, solvents, and catalysts utilized to synthesise the compounds of the present invention are either commercially available or can be produced by organic synthesis methods known to one of ordinary skill in the art. In all of the methods it is understood that protecting groups for sensitive or reactive groups may be employed where necessary in accordance with general principles of chemistry. Protecting groups are manipulated according to standard methods of organic synthesis (T. W. Green and P. G. M. Wuts (2014) Protective Groups in Organic Synthesis, 5th edition, John Wiley & Sons). These groups are removed at a convenient stage of the compound synthesis using methods that are readily apparent to those skilled in the art. Unless otherwise noted, reagents and solvents were used as received from commercial suppliers.

The chemical names were generated using ChemDraw Professional v19.1.2.36 from PerkinElmer.

Temperatures are given in degrees Celsius. As used herein, unless specified otherwise, the term “room temperature” or “ambient temperature” means a temperature of from 15° C. to 30° C., such as from 20° C. to 30° C., such as from 20° C. to 25° C. If not mentioned otherwise, all evaporations are performed under reduced pressure, typically between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesize the compounds of the present invention are either commercially available or can be produced by organic synthesis methods known to one of ordinary skill in the art.

In the following general methods, Y¹, Y², m, n, R¹, R², R³, R⁴ and

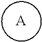

are as previously defined in the above embodiments, or limited to designations in the Schemes. PG is a suitable protecting group. Unless otherwise stated, starting materials are either commercially available or are prepared by known methods.

### List of Abbreviations

DIPEA (N,N-diisopropylethylamie)
Boc (tert-butyl carbamate)
IPA (isopropyl alcohol)
NBS (N-bromosuccinimde)
TEA (Triethylamine)
AcOH (Acetic Acid)
DMF (N,N-dimethylformamide)
NaH (Sodium hydride)
ACN (Acetonitrile)
THE (Tetrahydrofuran)
TBDMSCl (tertbutyldimethylsilyl chloride)
tBuOH (tert-butyl alcohol)
SFC (Supercritical fluid chromatography)
SEM (2-(Trimethylsilyl)ethoxymethyl)
MeOH (Methanol)
DIBAL-H (Diisobutylaluminum hydride)
m-CPBA (meta-chloroperoxybenzoic acid)
NaOtBu (Sodium tert-butoxide)
NaOAc (Sodium acetate)
NaBH₄ (Sodium borohydride)
NH₄Cl (ammonium chloride)
MeI (Methyliodide)
Cs₂CO₃ (Cesium Carbonate)
EtOH (Ethanol)
DMS (Dimethyl sulfate)
AC₂O (Acetic anhydride)
Zn (Zinc)

### General Synthetic Schemes

The examples were prepared as shown in Scheme 1 (5 membered rings) or Scheme 2 (6 membered rings).

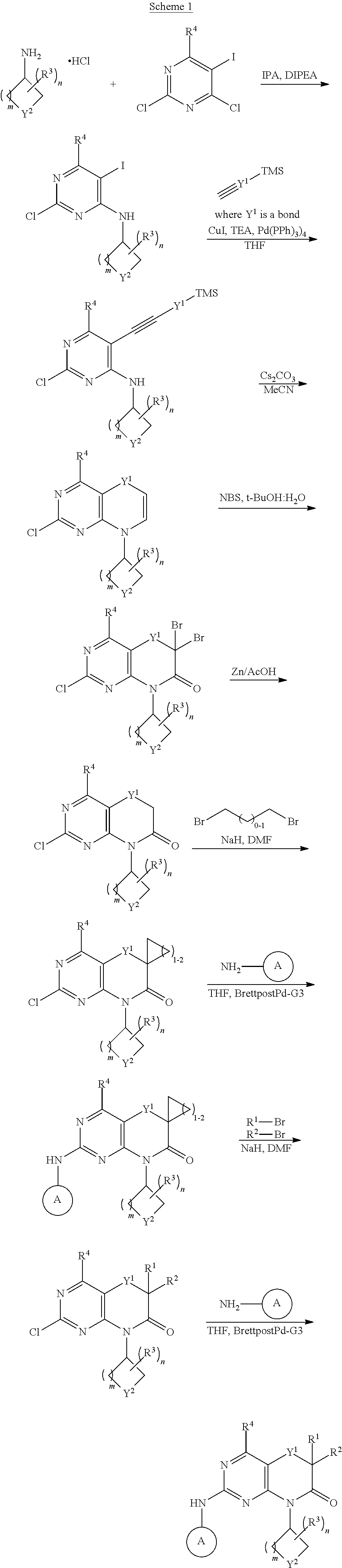

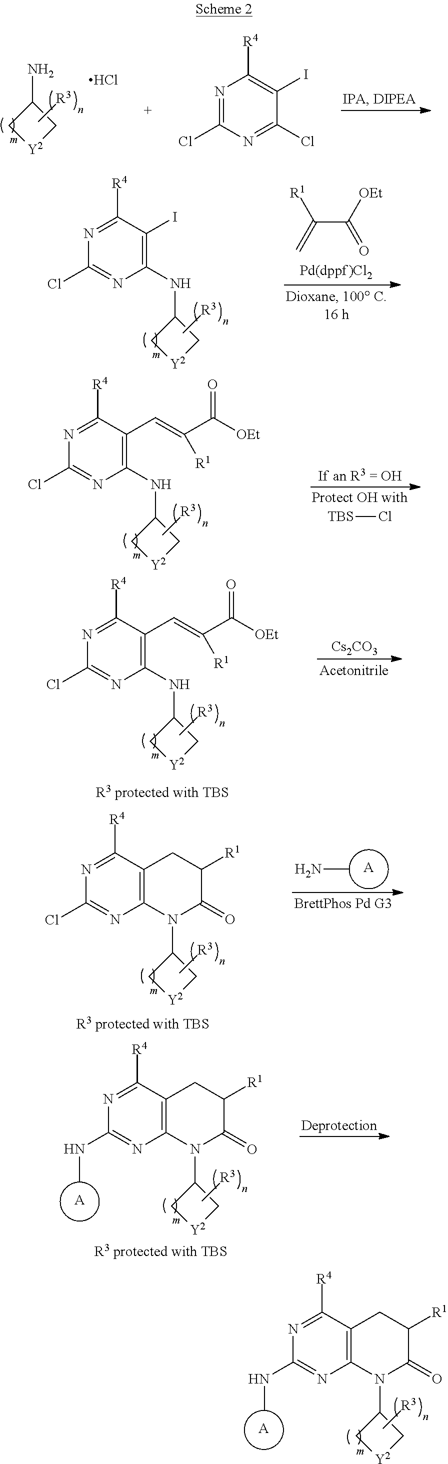

### Intermediate 1

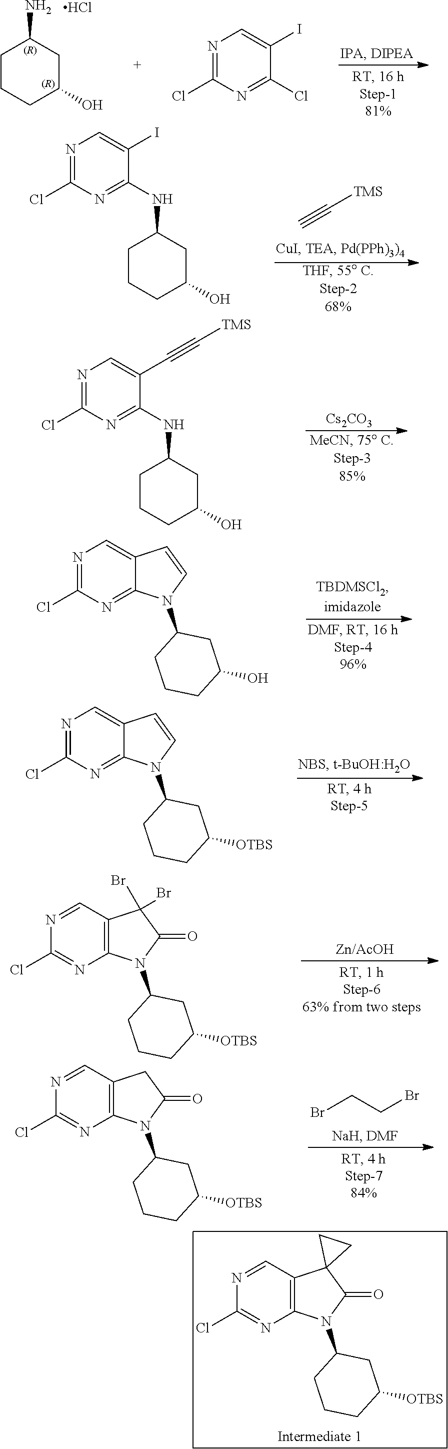

### Experimental Details of Intermediate 1

Step-1: Synthesis of (1R,3R)-3-((2-chloro-5-iodopyrimidin-4-yl)amino)cyclohexan-1-ol: To a stirred solution of (1R,3R)-3-aminocyclohexan-1-ol hydrochloride (60 g, 397.3 mmol) and DIPEA (183 mL, 993.25 mmol) in isopropanol (300 ml), was added 2,4-dichloro-5-iodopyrimidine (108 g, 397.3 mmol) at room temperature and stirred for 16 h. The progress of the reaction was monitored by TLC and LCMS.

After completion of the reaction, the solvent was removed under reduced pressure to give the crude product. The crude compound was purified by column chromatography by using silica gel (100-200 mesh), eluting with 0-40% ethyl acetate in pet-ether to give (1R,3R)-3-((2-chloro-5-iodopyrimidin-4-yl)amino)cyclohexan-1-ol (113 g, 81% yield, regio-isomeric mixture; 82% and 15% isomers) as pale yellow solid. LC-MS m/z [M+H]+=353.98, ¹H NMR (400 MHz, DMSO-d₆) δ=8.28-8.40 (m, 1H), 6.58 (d, J=8.4 Hz, 1H), 4.50-4.51 (m, 1H), 4.25-4.30 (m, 1H), 3.90-4.20 (m, 1H), 1.71-1.80 (m, 4H), 1.20-1.70 (m, 5H).

Step-2: Synthesis of (1R,3R)-3-((2-chloro-5-((trimethylsilyl)ethynyl)pyrimidin-4-yl)amino)cyclohexan-1-ol: To a degassed solution of (1R,3R)-3-((2-chloro-5-iodopyrimidin-4-yl)amino)cyclohexan-1-ol (56.5 g, 160.5 mmol; 82% and 15% isomers), CuI (3.04 g, 16.0 mmol), triethyl amine (115 mL, 800 mmol) and Pd(PPh₃)₄ (1.84 g, 1.60 mmol) in dry THE (560 mL), was added trimethylsilyl acetylene (22.7 mL, 160.5 mmol) and heated at 55° C. for 4 h. The progress of the reaction was monitored by TLC & LCMS. After completion of the reaction, the reaction mass was filtered through plug of celite, the filtrate was concentrated under reduced pressure to give the crude product. The crude compound was purified by column chromatography using silica gel (100-200 mesh), eluting with 0-20% ethyl acetate in pet-ether to give (1R,3R)-3-((2-chloro-5-((trimethylsilyl)ethynyl)pyrimidin-4-yl)amino)cyclohexan-1-ol (35 g, 68%) as pale yellow solid. LC-MS m/z [M+H]+=325.66, ¹H NMR (400 MHz, DMSO-d₆) δ=8.16 (s, 1H), 6.48 (d, J=8.0 Hz, 1H), 4.55 (d, J=2.80 Hz, 1H), 4.25-4.40 (m, 1H), 3.90-4.0 (m, 1H), 1.61-1.85 (m, 4H), 1.30-1.60 (m, 4H), 0.26 (s, 9H).

Step-3: Synthesis of (1R,3R)-3-(2-chloro-7H-pyrrolo[2,3-d] pyrimidin-7-yl) cyclohexan-1-ol: To a stirred solution of (1R,3R)-3-((2-chloro-5-((trimethylsilyl)ethynyl)pyrimidin-4-yl)amino)cyclohexan-1-ol (44 g, 136.2 mmol) in acetonitrile (2,200 mL), was added Cs₂CO₃ (22.1 g, 68.1 mmol) and heated at 75° C. for 6 h. Progress of the reaction was monitored by TLC and LCMS. After completion of the reaction, the reaction mass was filtered, the filtrate was concentrated under reduced pressure to give crude product. The crude compound was purified by column chromatography using silica gel (100-200 mesh), eluting with 0-50% ethyl acetate in pet-ether to give (1R,3R)-3-(2-chloro-7H-pyrrolo[2,3-d] pyrimidin-7-yl) cyclohexan-1-ol (29 g, 85% yield) as pale yellow solid; LC-MS m/z [M+H]+=252.17, ¹H NMR (400 MHz, DMSO-d₆) δ=8.90 (s, 1H), 7.82 (d, J=3.6 Hz, 1H), 6.70 (d, J=3.6 Hz, 1H), 4.75 (d, J=2.8 Hz, 1H), 4.15 (d, J=2.4 Hz, 1H), 2.01-2.11 (m, 1H), 1.40-1.99 (m, 7H).

Step-4: Synthesis of 7-((1R,3R)-3-((tert-butyldimethylsilyl) oxy)cyclohexyl)-2-chloro-7H-pyrrolo[2,3-d]pyrimidine: To a stirred solution of (1R,3R)-3-(2-chloro-7H-pyrrolo[2,3-d] pyrimidin-7-yl) cyclohexan-1-ol (20 g, 79.68 mmol) and imidazole (10.8 g, 159.3 mmol) in DMF (200 mL), was added TBDMS-Cl (18 g, 159.3 mmol) at room temperature and stirred for 16 h. Progress of the reaction was monitored by TLC and LCMS. After, 16 h, the reaction mass was quenched with ice-cold water (50 mL) and extracted with ethyl acetate (3×100 mL). The combined organic layers were washed with ice-cold water (2×60 mL). The organic layer was dried over Na₂SO₄, concentrated under reduced pressure. The crude compound was purified by column chromatography using silica gel (100-200 mesh), eluting with 0-50% ethyl acetate in pet-ether to give 7-((1R,3R)-3-((tert-butyldimethylsilyl) oxy)cyclohexyl)-2-chloro-7H-pyrrolo[2,3-d]pyrimidine (29 g, 96% yield) as brown gummy liquid. LC-MS m/z [M+H]⁺=366.24, ¹H NMR (400 MHz, DMSO-d₆) δ=8.88 (s, 1H), 7.79 (d, J=3.6 Hz, 1H), 6.68 (d, J=3.6 Hz, 1H), 4.95-5.10 (m, 1H), 4.27 (s, 1H), 1.96-2.05 (m, 1H), 1.80-1.95 (m, 4H), 1.64 (d, J=8.0 Hz, 2H), 1.45-1.55 (m, 1H), 0.90-0.98 (m, 9H), 0.01-0.50 (m, 6H),

Step-5: Synthesis of 5,5-dibromo-7-((1R,3R)-3-((tert-butyldimethylsilyl) oxy)cyclohexyl)-2-chloro-5,7-dihydro-6H-pyrrolo[2,3-d]pyrimidin-6-one: To a stirred solution of 7-((1R,3R)-3-((tert-butyldimethylsilyl) oxy)cyclohexyl)-2-chloro-7H-pyrrolo[2,3-d]pyrimidine (50 g, 136.9 mmol) in t-BuOH:H₂O (600 mL, 4:1), was added NBS (73 g, 410.9 mmol) at room temperature and stirred for 4 h. Progress of the reaction was monitored by TLC & LCMS. After completion of the reaction. The reaction mass was quenched with water (100 mL), extracted with MTBE (3×300 mL). The organic layer was dried over Na₂SO₄, concentrated under reduced pressure to get the crude 5,5-dibromo-7-((1R,3R)-3-((tert-butyldimethylsilyl) oxy)cyclohexyl)-2-chloro-5,7-dihydro-6H-pyrrolo[2,3-d]pyrimidin-6-one (73 g crude) as pale yellow semi-solid. The crude product was used as such for next step without further purification. LC-MS m/z [M+H]+=540.21

Step-6: Synthesis of 7-((1R,3R)-3-((tert-butyldimethylsilyl)oxy)cyclohexyl)-2-chloro-5,7-dihydro-6H-pyrrolo[2,3-d]pyrimidin-6-one: To a stirred solution of 5,5-dibromo-7-((1R,3R)-3-((tert-butyldimethylsilyl) oxy)cyclohexyl)-2-chloro-5,7-dihydro-6H-pyrrolo[2,3-d]pyrimidin-6-one (73 g crude, 135.4 mmol) in AcOH (730 mL), was added Zn dust (44 g, 677.1 mmol) at room temperature and stirred for 1 h. Progress of the reaction was monitored by TLC & LCMS. After completion of the reaction, the reaction mass was filtered through celite pad, the filtrate was diluted with ethyl acetate (500 mL) and washed with water (100 mL), the organic layer was dried over Na₂SO₄, concentrated under reduced pressure to get the crude product. The crude product was purified by using column chromatography (silica gel, 100-200 mesh), eluting with 0-30% ethyl acetate:pet ether to give 7-((1R,3R)-3-((tert-butyldimethylsilyl)oxy)cyclohexyl)-2-chloro-5,7-dihydro-6H-pyrrolo[2,3-d]pyrimidin-6-one (33 g, 63% yield after 2 steps) as brown gummy liquid. LC-MS m/z [M+H]+=382.29, ¹H NMR (400 MHz, DMSO-d₆) δ=8.25 (s, 1H), 4.58-4.68 (m, 1H), 4.26 (s, 1H), 3.63 (s, 2H), 2.30-2.40 (m, 1H), 2.10-2.21 (m, 1H), 1.55-1.80 (m, 5H), 1.45-1.50 (m, 1H), 0.91 (s, 9H), 0.01-0.10 (m, 6H).

Step-7: Synthesis of 7′-((1R,3R)-3-((tert-butyldimethylsilyl)oxy)cyclohexyl)-2′-chlorospiro[cyclopropane-1,5′-pyrrolo[2,3-d]pyrimidin]-6′(7′H)-one: To a stirred solution of 7-((1R,3R)-3-((tert-butyldimethylsilyl)oxy)cyclohexyl)-2-chloro-5,7-dihydro-6H-pyrrolo[2,3-d]pyrimidin-6-one (11 g, 28.87 mmol) and dibromoethane (7.4 mL, 86.61 mmol) in dry DMF (110 mL), was added NaH (60%, 3.4 g, 86.61 mmol) portion wise at room temperature and stirred for 4 h. Progress of the reaction was monitored by TLC & LCMS. After completion of the reaction, the reaction was quenched sat. NH₄Cl, extracted with MTBE (3×100 mL). The organic layer was dried over Na₂SO₄, concentrated under reduced pressure to get the crude product. The crude product was purified by column chromatography (silica gel, 100-200 mesh), eluting with 0-15% ethyl acetate in pet ether to give 7′-((1R,3R)-3-((tert-butyldimethylsilyl)oxy)cyclohexyl)-2′-chlorospiro[cyclopropane-1,5′-pyrrolo[2,3-d]pyrimidin]-6′(7′H)-one (10 g, 85% yield) as dark brown gummy liquid. LC-MS m/z [M+H]⁺=408.31, ¹H NMR (400 MHz, DMSO-d₆) δ=8.16 (s, 1H), 4.65-4.75 (m, 1H), 4.26 (s, 1H), 3.58-3.62 (m, 1H), 2.30-2.42 (m, 1H), 2.15-2.25 (m, 1H), 1.81-1.91 (m, 2H), 1.55-1.80 (m, 7H), 1.55-1.80 (m, 7H), 0.91 (s, 9H), 1.39-1.49 (m, 1H), 0.04 (s, 6H).

### Intermediate 2

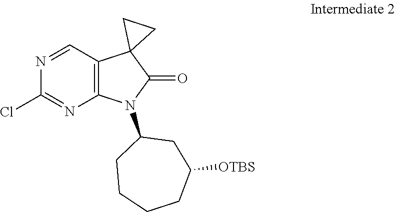 [SMILES: CC(C)(C)[Si](C)(C)O[C@@H]1CCCC[C@@H](N2C(=O)C3(CC3)c3cnc(Cl)nc32)C1]

Intermediate 2 was made using the method to generate intermediate 1, but starting from

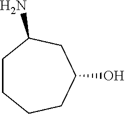

instead of

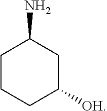

### Example 1

### Synthesis of

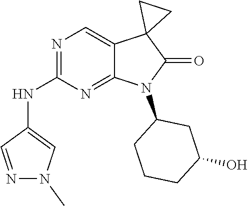 [SMILES: Cn1cc(Nc2ncc3c(n2)N([C@@H]2CCC[C@@H](O)C2)C(=O)C32CC2)cn1]

(7′-((1R,3R)-3-hydroxycyclohexyl)-2′-((1-methyl-1H-pyrazol-4-yl)amino)spiro[cyclopropane-1,5′-pyrrolo[2,3-d]pyrimidin]-6′(7′H)-one).

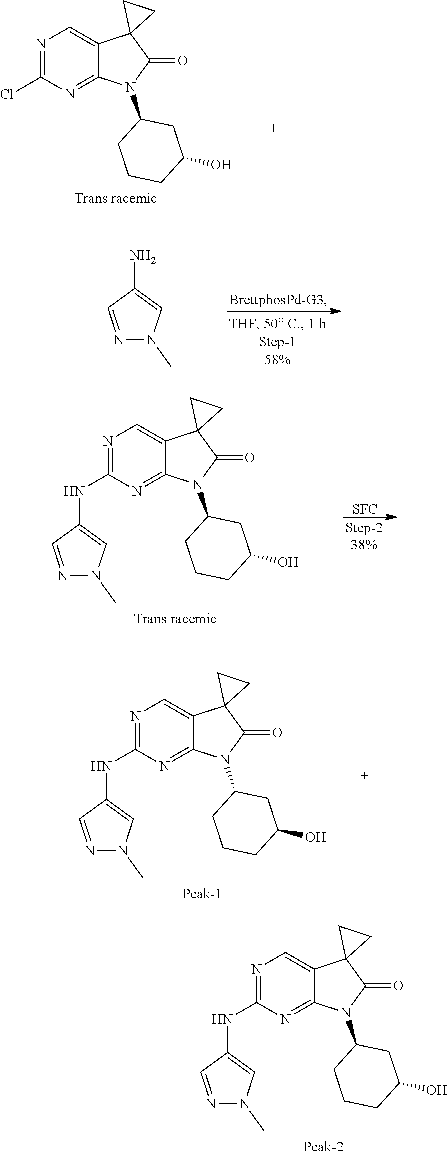

### Step-1: Synthesis of 7′-((1R,3R)-3-((tert-butyldimethylsilyl)oxy)cyclohexyl)-2′-((1-methyl-1H-pyrazol-4-yl)amino)spiro[cyclopropane-1,5′-pyrrolo[2,3-d]pyrimidin]-6′(7′H)-one (compound-2)

Procedure: To a degassed solution of 7′-3-((tert-butyldimethylsilyl)oxy)cyclohexyl)-2′-chlorospiro[cyclopropane-1,5′-pyrrolo[2,3-d]pyrimidin]-6′(7′H)-one (trans-racemic) (0.5 g, 1.70 mmol), 1-methyl-1H-pyrazol-4-amine (0.25 g, 2.55 mmol) and NaOtBu (0.24 g, 2.55 mmol) in dry THE (10 ml), was added Brettphospd-G3 (0.15 g, 0.17 mmol) under argon atmosphere and stirred at 50° C. for 1 h. The progress of the reaction was monitored by TLC. After completion of reaction, the reaction mass was filtered through celite pad, the filtrate was concentrated under reduced pressure to afford crude compound. The crude was purified by column chromatography (silica gel, 230-400 mesh), eluting with 0-5% methanol in DCM to give 7′-3-hydroxycyclohexyl)-2′-((1-methyl-1H-pyrazol-4-yl)amino)spiro[cyclopropane-1,5′-pyrrolo[2,3-d]pyrimidin]-6′(7′H)-one (trans-racemic) (0.35 g, 58% yield) as brown solid. LC-MS m/z [M+H]+=355.26, ¹H NMR (400 MHz, DMSO-d₆) δ=9.35 (s, 1H), 7.87 (s, 1H), 7.86 (s, 1H), 7.48 (s, 1H), 4.65-4.78 (m, 1H), 4.60 (s, 1H), 4.13 (s, 1H), 3.79 (s, 3H), 2.20-2.35 (m, 1H), 1.52-1.85 (m, 7H), 1.35-1.50 (m, 3H).

Step-2: SFC separation 7′-3-hydroxycyclohexyl)-2′-((1-methyl-1H-pyrazol-4-yl)amino)spiro[cyclopropane-1,5′-pyrrolo[2,3-d]pyrimidin]-6′(7′H)-one (trans-racemic) (0.35 g, 0.98 mmol) was subjected to SFC separation in methanol as co-solvent. After completion of the separation, the solvent of peak-2 was distilled off to give 7′-((1R,3R)-3-hydroxycyclohexyl)-2′-((1-methyl-1H-pyrazol-4-yl)amino)spiro[cyclopropane-1,5′-pyrrolo[2,3-d]pyrimidin]-6′(7′H)-one (0.136 g, 38%) as white solid. LC-MS m/z [M+H]+=355.30, ¹H NMR (400 MHz, DMSO-d₆) δ=9.35 (s, 1H), 7.87 (s, 1H), 7.85 (s, 1H), 7.48 (s, 1H), 4.55-4.80 (m, 2H), 4.13 (s, 1H), 3.79 (s, 3H), 2.40-2.50 (m, 1H), 2.20-2.35 (m, 1H), 1.51-1.85 (m, 7H), 1.35-1.50 (m, 3H).

### Example 2

### Synthesis of

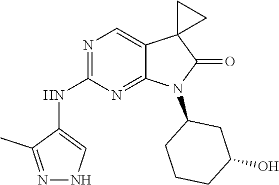 [SMILES: Cc1n[nH]cc1Nc1ncc2c(n1)N([C@@H]1CCC[C@@H](O)C1)C(=O)C21CC1]

(7′-((1R,3R)-3-hydroxycyclohexyl)-2′-((3-methyl-1H-pyrazol-4-yl)amino)spiro[cyclopropane-1,5′-pyrrolo[2,3-d]pyrimidin]-6′(7′H)-one).

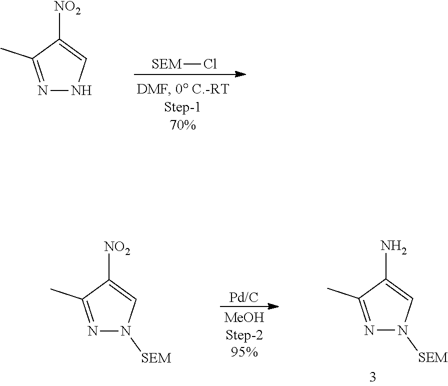

3-methyl-1-((2-(trimethylsilyl)ethoxy)methyl)-1H-pyrazol-4-amine: To a stirred solution of 3-methyl-4-nitro-1H-pyrazole (1 g, 7.86 mmol) and Cs₂CO₃ (5.12 g, 15.75 mmol) in DMF (10 ml), was added SEM-Cl (2.09 mL, 11.80 mmol) at 0° C. and then stirred at room temperature for 2 h. The progress of the reaction was monitored by TLC. After completion of reaction, the reaction mass was quenched with ice-cold water and extracted into ethyl acetate (2×100 mL). The combined organic layers were dried over anhydrous Na₂SO₄ and concentrated under reduced pressure to afford crude compound. The crude was purified by column chromatography (silica gel, 100-200 mesh), eluting with 0-10% ethyl acetate in pet ether to give 3-methyl-4-nitro-1-((2-(trimethylsilyl)ethoxy)methyl)-1H-pyrazole (1.43 g, 70% yield) as colorless liquid. ¹H NMR (400 MHz, DMSO-d₆) δ=8.03 (s, 1H), 8.32 (s, 1H), 5.58 (s, 1H), 5.44 (s, 2H), 3.58-3.68 (m, 4H), 2.70 (s, 2H), 2.49 (s, 3H), 0.85-0.93 (m, 4H), 0.0-0.04 (m, 9H).

Step-2: Synthesis of 53-methyl-1-((2-(trimethylsilyl)ethoxy)methyl)-1H-pyrazol-4-amine: To a stirred solution of 3-methyl-4-nitro-1-((2-(trimethylsilyl)ethoxy)methyl)-1H-pyrazole (1.43 g, 5.55 mmol) in MeOH (15 ml), was added Pd/C (0.2 g) at room temperature and stirred at hydrogen balloon pressure for 6 h. The progress of the reaction was monitored by TLC and LCMS. After completion of reaction, the reaction mass was filtered through celite pad, the filtrate was concentrated under reduced pressure and chased with toluene to afford crude 3-methyl-1-((2-(trimethylsilyl)ethoxy)methyl)-1H-pyrazol-4-amine (1.2 g, 95% yield) as pale red solid. The crude product was used as such for next step without further purification. ¹H NMR (400 MHz, DMSO-d₆) δ=6.90-7.038 (m, 1H), 5.21 (s, 1H), 5.11 (s, 1H), 3.68 (s, 2H), 3.38-3.45 (m, 2H), 1.90-2.10 (m, 3H), 0.79 (t, J=8.4 Hz, 2H), −0.05-0.04 (m, 9H).

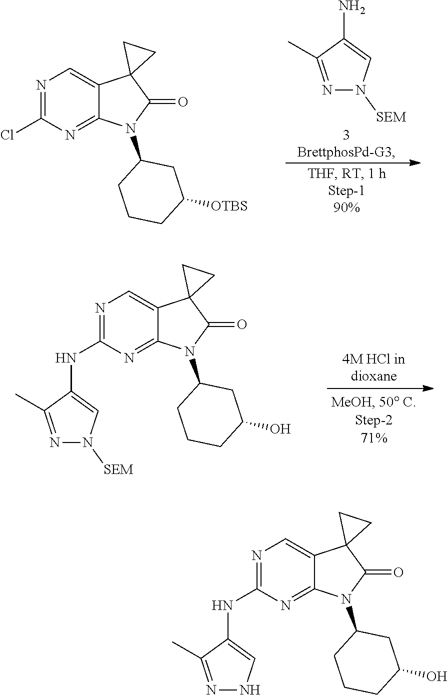

Step1: 7′-((1R,3R)-3-((tert-butyldimethylsilyl)oxy)cyclohexyl)-2′-((3-methyl-1-((2-(trimethylsilyl)ethoxy)methyl)-1H-pyrazol-4-yl)amino)spiro[cyclopropane-1,5′-pyrrolo[2,3-d]pyrimidin]-6′(7′H)-one: To a degassed solution of 7′-((1R,3R)-3-((tert-butyldimethylsilyl)oxy)cyclohexyl)-2′-chlorospiro[cyclopropane-1,5′-pyrrolo[2,3-d]pyrimidin]-6′(7′H)-one (30 g, 73.52 mmol) and 3-methy-1-((2-(trimethylsilyl)ethoxy)methyl)-1H-pyrazol-4-amine (18.2 g, 80.87 mmol) in dry THE (450 mL), was added NaOtBu (10.8 g, 110.28 mmol) and BrettphospdG3 (2.0 g, 2.2 mmol), reaction mass was degassed with argon for 5 min and the reaction mass was stirred at room temperature for 1 h. Progress of the reaction was monitored by TLC and LCMS. After completion of reaction, the reaction mass was filtered, the filtrate was concentrated under reduced pressure to give crude compound. The crude compound was purified by Sepa-Bean using silica gel (230-400 mesh), eluting with 0-30% ethyl acetate in pet-ether to give 7′-((1R,3R)-3-((tert-butyldimethylsilyl)oxy)cyclohexyl)-2′-((3-methyl-1-((2-(trimethylsilyl)ethoxy)methyl)-1H-pyrazol-4-yl)amino)spiro[cyclopropane-1,5′-pyrrolo[2,3-d]pyrimidin]-6′(7′H)-one. The obtained 7′-((1R,3R)-3-((tert-butyldimethylsilyl)oxy)cyclohexyl)-2′-((3-methyl-1-((2-(trimethylsilyl)ethoxy)methyl)-1H-pyrazol-4-yl)amino)spiro[cyclopropane-1,5′-pyrrolo[2,3-d]pyrimidin]-6′(7′H)-one was dissolved in DCM and stirred with Si-Thiourea (Palladium scavenger) for 3 hrs and filtered through celite pad. The filtrate was concentrated under reduced pressure to provide target compound as brown colour gummy solid (40 g, 90% yield).

$$LC - MS m / z {: [ M + H ]}^{+} = 599.77$$

Step2: 7′-((1R,3R)-3-hydroxycyclohexyl)-2′-((3-methyl-1H-pyrazol-4-yl)amino)spiro[cyclopropane-1,5′-pyrrolo[2,3-d]pyrimidin]-6′(7′H)-one: To a stirred solution of 7′-((1R,3R)-3-((tert-butyldimethylsilyl)oxy)cyclohexyl)-2′-((3-methyl-1-((2-(trimethylsilyl)ethoxy)methyl)-1H-pyrazol-4-yl)amino)spiro[cyclopropane-1,5′-pyrrolo[2,3-d]pyrimidin]-6′(7′H)-one (50 g, 83.48 mmol) in methanol (500 mL), was added 4M HCl in dioxane (250 mL) at room temperature and stirred at 50° C. for 4 h. Progress of the reaction was monitored by TLC & LCMS. After completion of the reaction, reaction mass was concentrated under reduced pressure. The crude product was basified with aq. NaHCO₃ and extracted with ethyl acetate (3×200 mL). The organic layer was dried over Na₂SO₄, concentrated under reduced pressure to get the crude compound. The crude product was washed with DCM:MTBE (10V, 1:4) and filtered. The obtained solid was dissolved in 50% MeCN in water and concentrated under reduced pressure to give 7-((1R,3R)-3-hydroxycyclohexyl)-2′-((3-methyl-1H-pyrazol-4-yl)amino)spiro[cyclopropane-1,5′-pyrrolo[2,3-d]pyrimidin]-6′(7′H)-one as off white solid (21 g, 71% yield). LC-MS m/z: [M+H]⁺=355.2 (96.47%)¹H NMR (400 MHz, DMSO-d₆) δ=12.27-12.18 (m, 1H), 8.59-8.54 (m, 1H), 7.81-7.55 (m, 2H), 4.66 (br s, 1H), 4.54 (br s, 1H), 4.08 (br s, 1H), 2.49 (s, 3H), 2.25-2.14 (m, 4H), 1.79-1.35 (m, 8H).

### Example 3

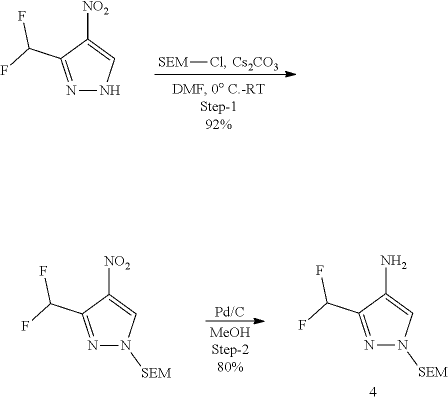

Step-1: Synthesis of 3-(difluoromethyl)-4-nitro-1-((2-(trimethylsilyl)ethoxy)methyl)-1H-pyrazole: To a stirred solution of 3-(difluoromethyl)-4-nitro-1H-pyrazole (1.5 g, 9.21 mmol) and Cs₂CO₃ (5.9 g, 18.42 mmol) in DMF (15 mL), was added SEM-Cl (2.5 mL, 13.81 mmol) at 0° C. and then stirred at room temperature for 2 h. The progress of the reaction was monitored by TLC and LCMS. After completion of reaction, reaction mass was quenched with ice-cold water and extracted with ethyl acetate (2×30 mL). The organic layer was washed with ice-cold water (2×10 mL), dried over Na₂SO₄ and concentrated under reduced pressure to give the crude product. The crude compound was purified by Sepa-Bean using silica gel (230-400 mesh), eluting with 0-10% ethyl acetate in pet-ether to give 3-(difluoromethyl)-4-nitro-1-((2-(trimethylsilyl)ethoxy)methyl)-1H-pyrazole (2.5 g, 92% yield) as colourless liquid. ¹H NMR (400 MHz, DMSO-d₆) δ=9.26 (s, 1H), 8.53 (s, 1H), 7.22-7.75 (m, 3H), 5.56 (s, 4H), 3.60-3.68 (m, 7H), 0.80-0.93 (m, 8H), 0.01-0.03 (m, 6H).

Step-2: Synthesis of 3-(difluoromethyl)-1-((2-(trimethylsilyl)ethoxy)methyl)-1H-pyrazol-4-amine (Intermediate-4): To a parr-shaker containing 3-(difluoromethyl)-4-nitro-1-((2-(trimethylsilyl)ethoxy)methyl)-1H-pyrazole (2.5 g, 8.53 mmol) in methanol (25 mL), was added Pd/C (10%, 0.25 g) and stirred at room temperature at 80 psi hydrogen pressure for 2 h. Progress of the reaction was monitored by TLC. After completion of reaction, reaction mass was filtered through plug of celite, the filtrate was concentrated under reduced pressure to give the crude product. The crude compound was purified by Sepa-Bean using silica gel (230-400 mesh), eluting with 0-20% ethyl acetate in pet-ether to give 3-(difluoromethyl)-1-((2-(trimethylsilyl)ethoxy)methyl)-1H-pyrazol-4-amine (1.8 g, 80% yield) as pale orange liquid. ¹H NMR (400 MHz, DMSO-d₆) δ=6.78-7.30 (m, 3H), 5.25-5.35 (m, 2H), 4.38 (br-s, 2H), 3.40-3.52 (m, 3H), 0.75-0.85 (m, 3H), −0.09-0.10 (m, 11H).

Coupling to Intermediate 1 and deprotection of SEM group to form

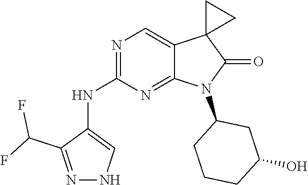 [SMILES: O=C1N([C@@H]2CCC[C@@H](O)C2)c2nc(Nc3c[nH]nc3C(F)F)ncc2C12CC2]

as 2′-((3-(difluoromethyl)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one per the method described in Example 2.

### Example 4

### Synthesis of

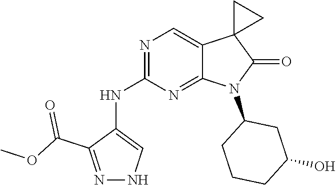 [SMILES: COC(=O)c1n[nH]cc1Nc1ncc2c(n1)N([C@@H]1CCC[C@@H](O)C1)C(=O)C21CC1]

(methyl 4-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)-1H-pyrazole-3-carboxylate).

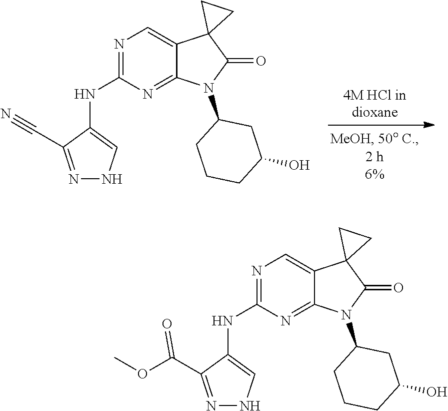 [SMILES: N#Cc1n[nH]cc1Nc1ncc2c(n1)N([C@@H]1CCC[C@@H](O)C1)C(=O)C21CC1 ; COC(=O)c1n[nH]cc1Nc1ncc2c(n1)N([C@@H]1CCC[C@@H](O)C1)C(=O)C21CC1]

Methyl 4-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)-1H-pyrazole-3-carboxylate: To a stirring solution of 4-((7′-((1R,3R)-3-hydroxycyclohexyl)-6′-oxo-6′,7-dihydrospiro[cyclopropane-1,5′-pyrrolo[2,3-d]pyrimidin]-2′-yl)amino)-1H-pyrazole-3-carbonitrile (0.25 g, 0.65 mmol) in methanol (2.5 ml), was added 4M HCl in 1,4-dioxne (1.5 mL) at 0° C. and then stirred at 50° C. for 2 h. The progress of the reaction was monitored by TLC & LCMS. After completion of reaction, the reaction mass was concentrated under reduced pressure, basified with sat. NaHCO₃ and extracted with ethyl acetate (3×10 mL). The organic layer was dried over Na₂SO₄ and concentrated under reduced pressure to afford crude compound. The crude was purified by Prep. HPLC (column: YMC triart (250*19 mm) 5u), Buffer A: 10 MM ABC Buffer B: acetonitrile, mobile phase conditions (% of B): 0/20,2/20,15/60,20/60,20.1/100,24/100,24.1/20,28/20 flow-19 mL APMS-022 to give methyl 4-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)-1H-pyrazole-3-carboxylate (15 mg, 6% yield) as off white solid.

LC-MS m/z [M+H]+=399.29, ¹H NMR (400 MHz, DMSO-d₆) δ=13.54 (br-s, 1H), 8.57 (br-s, 1H), 8.27 (s, 1H), 7.98 (s, 1H), 4.65-4.78 (m, 1H), 4.65 (d, J=2.4 Hz, 1H), 4.13 (s, 1H), 3.88 (s, 3H), 2.42-2.52 (m, 1H), 2.20-2.35 (m, 1H), 1.55-1.82 (m, 7H), 1.38-1.55 (m, 3H).

### Example 5

### Synthesis of

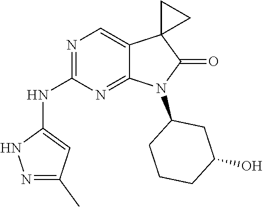 [SMILES: Cc1cc(Nc2ncc3c(n2)N([C@@H]2CCC[C@@H](O)C2)C(=O)C32CC2)[nH]n1]

(7′-((1R,3R)-3-hydroxycyclohexyl)-2′-((3-methyl-1H-pyrazol-5-yl)amino)spiro[cyclopropane-1,5′-pyrrolo[2,3-d]pyrimidin]-6′(7′H)-one).

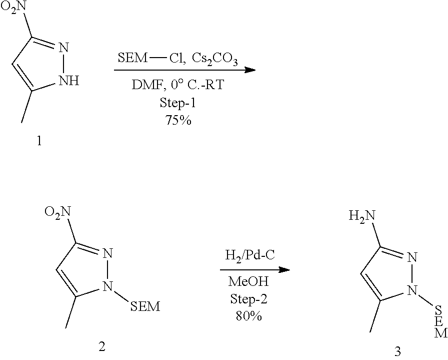

5-methyl-3-nitro-1-((2-(trimethylsilyl)ethoxy)methyl)-1H-pyrazole: To a stirred solution of 5-methyl-3-nitro-1H-pyrazole (2 g, 15.73 mmol) and Cs₂CO₃ (10.25 g, 31.47 mmol) in DMF (20 ml), was added SEM-Cl (4.14 mL, 23.60 mmol) at 0° C. and then stirred at room temperature for 2 h. The progress of the reaction was monitored by TLC. After completion of reaction, the reaction mass was quenched with ice-cold water and extracted into ethyl acetate (2×100 mL). The combined organic layers were dried over anhydrous Na₂SO₄ and concentrated under reduced pressure to afford crude compound. The crude was purified by column chromatography (silica gel, 100-200 mesh), eluting with 0-10% ethyl acetate in pet ether to give 5-methyl-3-nitro-1-((2-(trimethylsilyl)ethoxy)methyl)-1H-pyrazole (3 g, 75% yield) as pale yellow liquid. ¹H NMR (400 MHz, CDCl₃) δ=6.92 (s, 1H), 5.81 (s, 2H), 3.62 (dd, J=7.2, 8.8 Hz, 2H), 2.33 (s, 3H), 0.91 (t, 3H, J=8.0 Hz, 2H), 0.01 (s, 9H). 5-methyl-1-((2-(trimethylsilyl)ethoxy)methyl)-1H-pyrazol-3-amine: To a stirred solution of 5-methyl-3-nitro-1-((2-(trimethylsilyl)ethoxy)methyl)-1H-pyrazole (3 g, 11.65 mmol) in MeOH (30 ml), was added Pd/C (0.43 g) at room temperature and stirred at 80 psi H₂ pressure for 2 h. The progress of the reaction was monitored by TLC and LCMS. After completion of reaction, the reaction mass was filtered through celite pad, the filtrate was concentrated under reduced pressure and chased with toluene to afford crude 5-methyl-1-((2-(trimethylsilyl)ethoxy)methyl)-1H-pyrazol-3-amine (2.1 g, 80.7% yield) as pale red liquid. The crude product was used as such for next step without further purification. LC-MS m/z [M+H]+=228.42.

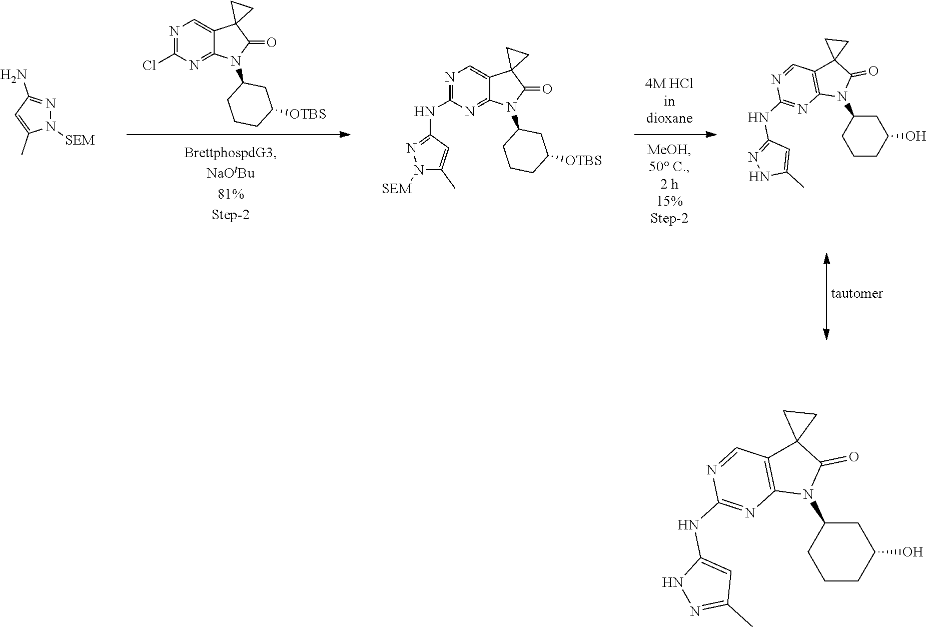 [SMILES: Cc1cc(N)nn1COCC[Si](C)(C)C ; CC(C)(C)[Si](C)(C)O[C@@H]1CCC[C@@H](N2C(=O)C3(CC3)c3cnc(Cl)nc32)C1 ; Cc1cc(Nc2ncc3c(n2)N([C@@H]2CCC[C@@H](O[Si](C)(C)C(C)(C)C)C2)C(=O)C32CC2)nn1COCC[Si](C)(C)C ; Cc1cc(Nc2ncc3c(n2)N([C@@H]2CCC[C@@H](O)C2)C(=O)C32CC2)n[nH]1 ; Cc1cc(Nc2ncc3c(n2)N([C@@H]2CCC[C@@H](O)C2)C(=O)C32CC2)[nH]n1]

Synthesis of rac7′-((1R,3R)-3-((tert-butyldimethylsilyl)oxy)cyclohexyl)-2′-((5-methyl-1-((2-(trimethylsilyl)ethoxy)methyl)-1H-pyrazol-3-yl)amino)spiro[cyclopropane-1,5′-pyrrolo[2,3-d]pyrimidin]-6′(7′H)-one: To a degassed solution of 7′-((1R,3R)-3-((tert-butyldimethylsilyl)oxy)cyclohexyl)-2′-chlorospiro[cyclopropane-1,5′-pyrrolo[2,3-d]pyrimidin]-6′(7′H)-one (0.5 g, 1.22 mmol), 5-methyl-1-((2-(trimethylsilyl)ethoxy)methyl)-1H-pyrazol-3-amine (0.33 g, 1.47 mmol) and NaOtBu (0.17 g, 1.83 mmol) in dry THE (10 ml), was added Brettphospd-G3 (0.1 g, 0.12 mmol) at room temperature and stirred for 2 h. The progress of the reaction was monitored by TLC. After completion of reaction, the reaction mass was filtered through celite pad, the filtrate was concentrated under reduced pressure to afford crude compound. The crude was purified by column chromatography (silica gel, 100-200 mesh), eluting with 0-30% ethyl acetate in pet ether to give 7′-((1R,3R)-3-((tert-butyldimethylsilyl)oxy)cyclohexyl)-2′-((5-methyl-1-((2-(trimethylsilyl)ethoxy)methyl)-1H-pyrazol-3-yl)amino)spiro[cyclopropane-1,5′-pyrrolo[2,3-d]pyrimidin]-6′(7′H)-one (0.57 g, 81% yield) as pale brown solid. LC-MS m/z [M+H]+=599.5 (21%+22%, positional isomers). 7′-((1R,3R)-3-hydroxycyclohexyl)-2′-((5-methyl-1H-pyrazol-3-yl)amino)spiro[cyclopropane-1,5′-pyrrolo[2,3-d]pyrimidin]-6′(7′H)-one: To a stirring solution of 7′-((1R,3R)-3-((tert-butyldimethylsilyl)oxy)cyclohexyl)-2′-((5-methyl-1-((2-(trimethylsilyl)ethoxy)methyl)-1H-pyrazol-3-yl)amino)spiro[cyclopropane-1,5′-pyrrolo[2,3-d]pyrimidin]-6′(7′H)-one (0.57 g, 0.95 mmol, 69% purity) in methanol (5 ml), was added 4M HCl in 1,4-dioxne (2.85 mL) at 0° C. and then stirred at 50° C. for 2 h. The progress of the reaction was monitored by TLC & LCMS. After completion of reaction, the reaction mass was concentrated under reduced pressure, basified with sat. NaHCO₃ and extracted with ethyl acetate (3×10 mL). The organic layer was dried over Na₂SO₄ and concentrated under reduced pressure to afford crude compound. The crude was purified by Prep. HPLC (column XB 19*150), Buffer A: ABC Buffer B: Acetonitrile, mobile phase conditions (% of B): 0/20,2/20,8/30,11/30 flow-30 ml/min dac to give 7-((1R,3R)-3-hydroxycyclohexyl)-2′-((5-methyl-1H-pyrazol-3-yl)amino)spiro[cyclopropane-1,5′-pyrrolo[2,3-d]pyrimidin]-6′(7′H)-one (50 mg, 15% yield) as off white solid. LC-MS m/z [M+H]+=355.30 (97.54%), ¹H NMR (400 MHz, DMSO-d₆) δ=11.79 (br-s, 1H), 9.45 (br-s, 1H), 7.86 (s, 1H), 6.34 (br-s, 1H), 4.63-4.73 (m, 1H), 4.57 (s, 1H), 4.11 (s, 1H), 2.50-2.60 (m, 1H), 2.25-2.38 (m, 1H), 2.19 (s, 3H), 1.51-1.85 (m, 7H), 1.35-1.50 (m, 3H).

### Example 6

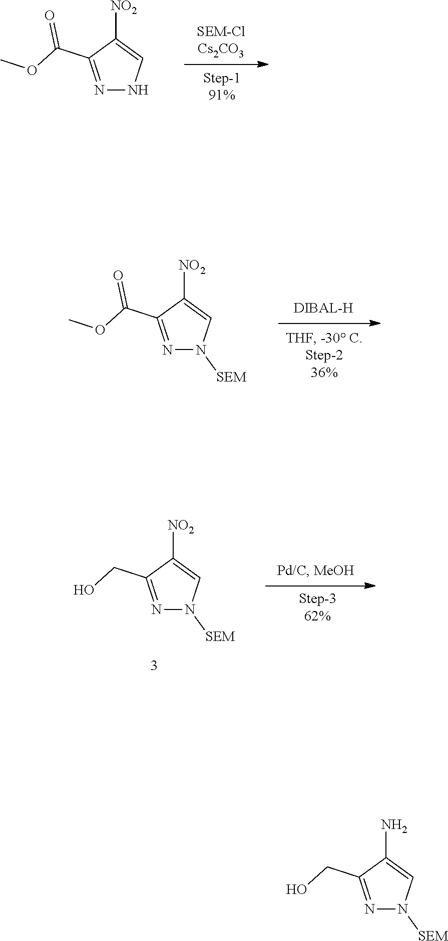 [SMILES: COC(=O)c1n[nH]cc1[N+](=O)[O-] ; COC(=O)c1nn(COCC[Si](C)(C)C)cc1[N+](=O)[O-] ; C[Si](C)(C)CCOCn1cc([N+](=O)[O-])c(CO)n1 ; C[Si](C)(C)CCOCn1cc(N)c(CO)n1]

Methyl 4-nitro-1-((2-(trimethylsilyl)ethoxy)methyl)-1H-pyrazole-3-carboxylate: To a stirred solution of methyl 4-nitro-1H-pyrazole-3-carboxylate (2 g, 11.69 mmol) and Cs₂CO₃ (7.6 g, 23.39 mmol) in DMF (20 mL), was added SEM-Cl (3.2 mL, 17.54 mmol) at 0° C. and then stirred at room temperature for 2 h. The progress of the reaction was monitored by TLC and LCMS. After completion of reaction, reaction mass was quenched with ice-cold water and extracted with ethyl acetate (2×30 mL). The organic layer was washed with ice-cold water (2×10 mL), dried over Na₂SO₄ and concentrated under reduced pressure to give the crude product. The crude compound was purified by Sepa-Bean using silica gel (230-400 mesh), eluting with 0-30% ethyl acetate in pet-ether to give methyl 4-nitro-1-((2-(trimethylsilyl)ethoxy)methyl)-1H-pyrazole-3-carboxylate (3.2 g, 91% yield) as colourless liquid. ¹H NMR (400 MHz, DMSO-d₆) δ=9.18 (s, 1H), 8.46 (s, 1H), 5.60 (s, 2H), 5.51 (s, 2H), 3.97 (s, 3H), 3.89 (s, 3H), 3.50-3.64 (m, 4H), 0.80-0.90 (m, 4H), −0.05-0.01 (m, 9H).

(4-nitro-1-((2-(trimethylsilyl)ethoxy)methyl)-1H-pyrazol-3-yl)methanol: To a stirred solution of methyl 4-nitro-1-((2-(trimethylsilyl)ethoxy)methyl)-1H-pyrazole-3-carboxylate (1.5 g, 4.97 mmol) in dry THE (15 ml) was added DIBAH (14.93 ml, 14.93 mmol) drop wise at −30° C., then reaction mass was raised to room temperature and stirred for 5 h. The progress of the reaction was monitored TLC & LCMS. After completion of the reaction, quenched with 1N HCl and extracted with ethyl acetate (3×30 mL). The organic layer was dried over Na₂SO₄, concentrated to give crude compound. The crude compound was purified by Sepa-Bean using silica gel (230-400 mesh), eluting with 0-50% ethyl acetate in pet-ether to give (4-nitro-1-((2-(trimethylsilyl)ethoxy)methyl)-1H-pyrazol-3-yl)methanol (0.5 g, 36% yield) as pale brown liquid. ¹H NMR (400 MHz, CDCl₃) δ=8.32 (s, 1H), 5.41 (s, 2H), 4.93 (d, J=5.6 Hz, 2H), 3.64 (t, J=8.4 Hz, 2H), 2.83 (br-s, 1H), 0.95 (t, J=8.4 Hz, 2H), −0.08-0.01 (m, 9H).

4-amino-1-((2-(trimethylsilyl)ethoxy)methyl)-1H-pyrazol-3-yl)methanol: To a stirring solution of (4-nitro-1-((2-(trimethylsilyl)ethoxy)methyl)-1H-pyrazol-3-yl)methanol (0.46 g, 8.53 mmol) in methanol (10 mL), was added Pd/C (10%, 0.1 g) and stirred at room temperature under hydrogen balloon pressure for 2 h. Progress of the reaction was monitored by TLC. After completion of reaction, reaction mass was filtered through plug of celite, the filtrate was concentrated under reduced pressure to give the crude 4-amino-1-((2-(trimethylsilyl)ethoxy)methyl)-1H-pyrazol-3-yl)methanol (0.25 g, 62% yield) as pale green liquid. The crude product used as such without further purification. ¹H NMR (400 MHz, CDCl₃) δ=7.14 (s, 1H), 5.27 (s, 2H), 4.74 (s, 2H), 3.50-3.60 (m, 2H), 0.88-1.0 (m, 2H), −0.01-0.05 (m, 9H).

Coupling to Intermediate 1 and deprotection of SEM group to form 7′-((1R,3R)-3-hydroxycyclohexyl)-2′-((3-(hydroxymethyl)-1H-pyrazol-4-yl)amino)spiro[cyclopropane-1,5′-pyrrolo[2,3-d]pyrimidin]-6′(7′H)-one

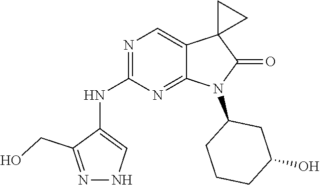 [SMILES: O=C1N([C@@H]2CCC[C@@H](O)C2)c2nc(Nc3c[nH]nc3CO)ncc2C12CC2]

as per the method described in Example 2.

### Example 7

### Synthesis of

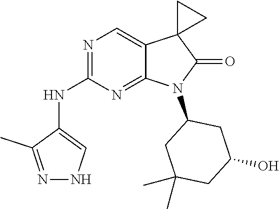 [SMILES: Cc1n[nH]cc1Nc1ncc2c(n1)N([C@H]1C[C@H](O)CC(C)(C)C1)C(=O)C21CC1]

(7′-((1R,5R)-5-hydroxy-3,3-dimethylcyclohexyl)-2′-((3-methyl-1H-pyrazol-4-yl)amino)spiro[cyclopropane-1,5′-pyrrolo[2,3-d]pyrimidin]-6′(7′H)-one).

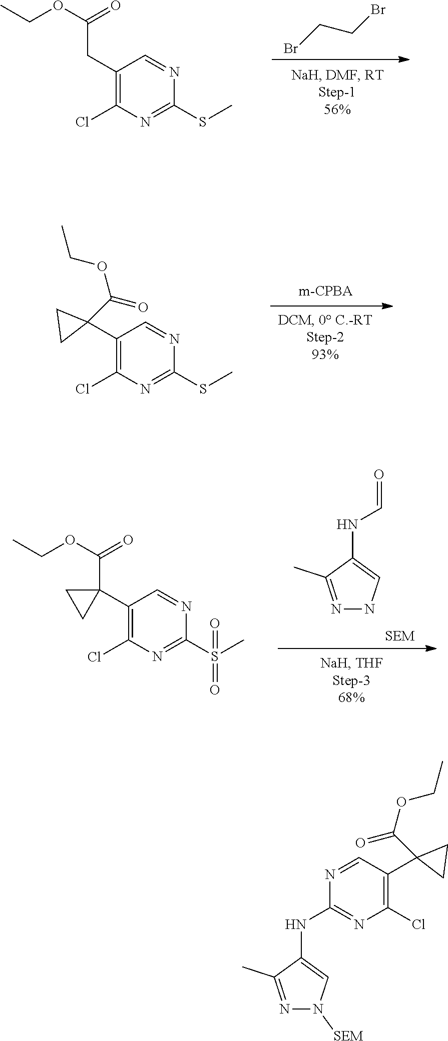

Ethyl 1-(4-chloro-2-(methylthio)pyrimidin-5-yl)cyclopropane-1-carboxylate: To a stirring solution of (commercially sourced) ethyl 2-(4-chloro-2-(methylthio)pyrimidin-5-yl)acetate (9 g, 36.48 mmol) and dibromoethane (15.7 mL, 182.40 mmol) in dry DMF, was added NaH (60%, 7.2 g, 182.4 mmol) portion wise at room temperature and stirred for 2 h. The progress of the reaction was monitored by TLC & LCMS. After completion of reaction, the reaction mass was quenched with ice-cold water, extracted with ethyl acetate (2×30 mL). The combined organic layers were washed with ice-cold water (2×10 mL) and brine solution (10 mL). The organic layer was dried over Na₂SO₄, concentrated under reduced pressure to afford crude compound. The crude was purified by flash column chromatography (230-400 g silica gel), eluting with 0-10% ethyl acetate in pet ether to give ethyl 1-(4-chloro-2-(methylthio)pyrimidin-5-yl)cyclopropane-1-carboxylate (5.6 g, 56% yield) as pale yellow liquid. LC-MS m/z [M+H]+=279.03, ¹H NMR (400 MHz, DMSO-d₆) δ=8.62 (s, 1H), 4.05 (q, J=6.8 Hz, 2H), 2.54 (s, 3H), 1.55-1.60 (m, 2H), 1.32-1.38 (m, 2H), 1.1 (t, J=6.8 Hz, 3H).

Ethyl 1-(4-chloro-2-(methylsulfonyl)pyrimidin-5-yl)cyclopropane-1-carboxylate; To a stirring solution of ethyl 1-(4-chloro-2-(methylthio)pyrimidin-5-yl)cyclopropane-1-carboxylate (5.6 g, 20.53) in DCM (84 ml), was added m-CPBA (15.18 g, 61.59 mmol) portion wise at 0° C. and then stirred at room temperature for 2 h. The progress of the reaction was monitored by TLC & LCMS. After completion of reaction, the reaction mass was quenched with sat. NaHCO₃, extracted with DCM (2×100 mL), dried over Na₂SO₄, concentrated under reduced pressure to afford crude compound. The crude was purified by flash column chromatography (230-400 g silica gel), eluting with 0-20% ethyl acetate in pet ether to give Ethyl 1-(4-chloro-2-(methylsulfonyl)pyrimidin-5-yl)cyclopropane-1-carboxylate (5.8 g, 93% yield) as white solid. LC-MS m/z [M+H]+=305.09, ¹H NMR (400 MHz, DMSO-d₆) δ=9.10 (s, 1H), 4.08 (q, J=6.8 Hz, 2H), 3.46 (s, 3H), 1.65-1.70 (m, 2H), 1.45-1.52 (m, 2H), 1.1 (t, J=6.8 Hz, 3H).

Ethyl1-(4-chloro-2-((3-methyl-1-((2-(trimethylsilyl)ethoxy)methyl)-1H-pyrazol-4-yl)amino)pyrimidin-5-yl)cyclopropane-1-carboxylate: To a stirring solution of Ethyl 1-(4-chloro-2-(methylsulfonyl)pyrimidin-5-yl)cyclopropane-1-carboxylate (6.5 g, 21.42 mmol) and N-(3-methyl-1-((2-(trimethylsilyl)ethoxy)methyl)-1H-pyrazol-4-yl)formamide (5.47 g, 21.42 mmol) in dry THE (65 mL), was added NaH (60%, 1.02 g, 25.71 mmol) portion wise at room temperature and heated at 50° C. for 4 h. The progress of the reaction was monitored for by TLC & LCMS. After completion of reaction, the reaction mass was quenched with ice-cold water, extracted with ethyl acetate (2×100 mL). The combined organic layers were dried over Na₂SO₄, concentrated under reduced pressure to afford crude compound. The crude was purified by flash column chromatography (230-400 g silica gel), eluting with 0-20% ethyl acetate in pet ether to give ethyl 1-(4-chloro-2-((3-methyl-1-((2-(trimethylsilyl)ethoxy)methyl)-1H-pyrazol-4-yl)amino)pyrimidin-5-yl)cyclopropane-1-carboxylate (7 g, 68% yield) as brown gummy liquid. LC-MS m/z [M+H]+=452.34, ¹H NMR (400 MHz, DMSO-d₆) δ=9.36 (s, 1H), 8.36 (s, 1H), 8.01 (s, 1H), 5.32 (s, 2H), 4.08 (q, J=7.2 Hz, 2H), 3.55 (t, J=8.0 Hz, 2H), 2.17 (s, 3H), 1.52-1.62 (m, 2H), 1.25-1.35 (m, 2H), 1.14 (t, J=7.2 Hz, 3H), 0.86 (t, J=8.0 Hz, 3H), 0.01-0.0 (s, 9H).

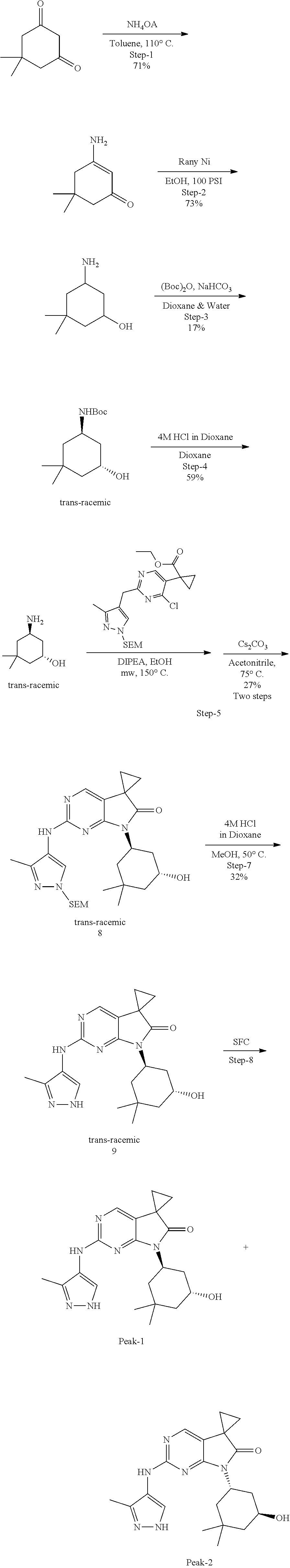

3-amino-5,5-dimethylcyclohex-2-en-1-one: In to a two neck round bottom flask equipped with dean-stark apparatus containing 5,5-dimethylcyclohexane-1,3-dione (5 g, 35.66 mmol) in toluene (100 ml), were added acetic acid (0.9 mL, 16.05 mmol) and NH₄OAc (5.5 g, 71.32 mmol), stirred at 110° C. for 12 h. The progress of the reaction was monitored by TLC and LCMS. After completion of reaction, the reaction mass was quenched with sat. Na₂CO₃ (10 mL) and 1N NaOH (10 mL) and extracted with DCM (10×20 mL). The organic layer was dried over Na₂SO₄, concentrated under reduced pressure to afford crude 3-amino-5,5-dimethylcyclohex-2-en-1-one (3.5 g, 71% yield) as pale yellow solid. The crude product was used as such for next step without further purification. ¹H NMR (400 MHz, DMSO-d₆) δ=6.71 (br-s, 2H), 4.89 (s, 1H), 2.12 (s, 2H), 1.90 (s, 2H), 0.96 (s, 6H). 5-amino-3,3-dimethylcyclohexan-1-ol: To a stirred solution of 3-amino-5,5-dimethylcyclohex-2-en-1-one (4 g, 28.23 mmol) in ethanol (40 ml), was added 20% NaOH (1.2 mL) and Raney-Ni (3.6 g) and stirred at 100 psi hydrogen pressure for 48 h. The progress of the reaction was monitored by TLC and LCMS. After completion of reaction, the reaction mass was filtered through celite pad, the filtrate was concentrated under reduced pressure and chased with toluene to afford crude 5-amino-3,3-dimethylcyclohexan-1-ol (3 g, 73% yield) as brown liquid. The crude product was used as such for next step without further purification. ¹H NMR (400 MHz, CDCl3) δ=4.35-4.52 (brs, 1H), 4.20-4.28 (m, 1H), 3.68-3.74 (m, 2H), 3.20-3.30 (m, 1H), 2.17 (d, J=8.4 Hz, 4H), 1.88-2.0 (m, 2H), 1.20-1.70 (m, 22H).

Tert-butyl ((1R,5R)-5-hydroxy-3,3-dimethylcyclohexyl) carbamate: To a stirred solution of tert-butyl ((1R,5R)-5-hydroxy-3,3-dimethylcyclohexyl) carbamate (10 g, 69.81 mmol) in dioxane (50 ml), and water (50 mL), were added NaHCO₃ (5.8 g, 69.81 mmol) and (Boc)₂O (19.2 mL, 83.78 mmol) and stirred at room temperature 2 h. The progress of the reaction was monitored by TLC and LCMS. After completion of reaction, the reaction mass was quenched with sat. NaCl and extracted with DCM (3×100 mL). The organic layer was dried over Na₂SO₄, concentrated under reduced pressure to afford crude compound. The crude was purified by column chromatography (silica gel, 100-200 mesh), eluting with 0-40% ethyl acetate in pet ether to give tert-butyl ((1R,5R)-5-hydroxy-3,3-dimethylcyclohexyl) carbamate (3.5 g, 17% yield) as pale yellow liquid. ¹H NMR (400 MHz, CDCl3) δ=4.38 (br-s, 1H), 4.19 (s, 1H), 3.98 (br-s, 1H), 1.92-2.02 (m, 1H), 1.68-1.75 (m, 2H), 1.50-1.55 (m, 2H), 1.40-1.49 (m, 10H), 1.30-1.40 (m, 2H), 1.15 (s, 3H), 1.02-1.12 (m, 1H), 0.90-0.98 (m, 4H).

(1R,5R)-5-amino-3,3-dimethylcyclohexan-1-ol hydrochloride: To a stirred solution of compound-4A (3 g, 12.34 mmol) in 1,4-dioxane (15 ml), was 4M HCl in dioxane (15 mL) at room temperature and stirred 2 h. The progress of the reaction was monitored by TLC and LCMS. After completion of reaction, the solvent was removed under reduced pressure, triturated with diethyl ether, decanted and dried under reduced pressure to afford crude (1R,5R)-5-amino-3,3-dimethylcyclohexan-1-ol hydrochloride (1.6 g, 59% yield) as off white solid. The crude product was used as such for next step without further purification. LC-MS m/z [M+H]+=144.29.

Rac7′-((1R,5R)-5-hydroxy-3,3-dimethylcyclohexyl)-2′-((3-methyl-1-((2-(trimethylsilyl)ethoxy)methyl)-1H-pyrazol-4-yl)amino)spiro[cyclopropane-1,5′-pyrrolo[2,3-d]pyrimidin]-6′(7′H)-one: To a stirred solution of (1R,5R)-5-amino-3,3-dimethylcyclohexan-1-ol hydrochloride (0.6 g, 3.32 mmol) and DIPEA (1.2 mL, 6.65 mmol) in ethanol (20 mL), was added ethyl 1-(4-chloro-2-((3-methyl-1-((2-(trimethylsilyl)ethoxy)methyl)-1H-pyrazol-4-yl)amino)pyrimidin-5-yl)cyclopropane-1-carboxylate (1 g, 2.21 mmol) and irradiated under microwave radiation at 150° C. for 4 h.

After completion of reaction, the solvent was removed under reduced pressure to give crude SnAr adduct, which was dissolved in acetonitrile (10 mL) and added Cs₂CO₃ (0.29 g, 0.89 mmol) and heated at 75° C. for 6 h. Progress of the reaction was monitored by LCMS. After completion of reaction, the reaction mass was filtered, the filtrate was concentrated to give crude compound. The crude product was purified by column chromatography (silica gel, 100-200 mesh), eluting with 0-70% ethyl acetate in pet ether to give rac7′-((1R,5R)-5-hydroxy-3,3-dimethylcyclohexyl)-2′-((3-methyl-1-((2-(trimethylsilyl)ethoxy)methyl)-1H-pyrazol-4-yl)amino)spiro[cyclopropane-1,5′-pyrrolo[2,3-d]pyrimidin]-6′(7′H)-one (0.3 g, 27% yield after 2 steps) as pale yellow liquid. LC-MS m/z [M+H]+=513.47.

Rac7′-((1R,5R)-5-hydroxy-3,3-dimethylcyclohexyl)-2′-((3-methyl-1H-pyrazol-4-yl)amino)spiro[cyclopropane-1,5′-pyrrolo[2,3-d]pyrimidin]-6′(7′H)-one: To a stirred solution of rac7′-((1R,5R)-5-hydroxy-3,3-dimethylcyclohexyl)-2′-((3-methyl-1-((2-(trimethylsilyl)ethoxy)methyl)-1H-pyrazol-4-yl)amino)spiro[cyclopropane-1,5′-pyrrolo[2,3-d]pyrimidin]-6′(7′H)-one (0.3 g, 0.58 mmol) in MeOH (1.5 ml), was added 4M HCl in dioxane (1.5 mL) and heated at 50° C. for 3 h. The progress of the reaction was monitored by TLC and LCMS. After completion of reaction, the reaction mass concentrated, basified with aq. NaHCO₃ and extracted with ethyl acetate, the organic layer was dried over Na₂SO₄, concentrated under reduced pressure to afford crude compound. The crude was purified by Prep. HPLC (Buffer A: 10 mm abc, Buffer B:—ACN solubility:—ACN-THF-water mobile phase conditions (% of B):—0/30,2/30,10/55,15/55,15.01/100,18/100,18.01/30,21/30 flow-10 ml/min anl-mcl-prep-014 column: XB c-18(19*250 mm) Sum buffer) to give trans-rac7′-((1R,5R)-5-hydroxy-3,3-dimethylcyclohexyl)-2′-((3-methyl-1H-pyrazol-4-yl)amino)spiro[cyclopropane-1,5′-pyrrolo[2,3-d]pyrimidin]-6′(7′H)-one (70 mg, 32% yield) as off white solid. LC-MS m/z [M+H]+=383.43

7′-((1R,5R)-5-hydroxy-3,3-dimethylcyclohexyl)-2′-((3-methyl-1H-pyrazol-4-yl)amino)spiro[cyclopropane-1,5′-pyrrolo[2,3-d]pyrimidin]-6′(7′H)-one: trans-racemic 7′-((1R,5R)-5-hydroxy-3,3-dimethylcyclohexyl)-2′-((3-methyl-1H-pyrazol-4-yl)amino)spiro[cyclopropane-1,5′-pyrrolo[2,3-d]pyrimidin]-6′(7′H)-one (70 mg) was subjected to SFC separation in methanol as co-solvent. After completion of the separation, the solvent of peak-1 was distilled off to give 7′-((1R,5R)-5-hydroxy-3,3-dimethylcyclohexyl)-2′-((3-methyl-1H-pyrazol-4-yl)amino)spiro[cyclopropane-1,5′-pyrrolo[2,3-d]pyrimidin]-6′(7′H)-one (22 mg) as off white solid. LC-MS m/z [M+H]+=383.43, ¹H NMR (400 MHz, DMSO-d₆) δ=12.15 (br-s, 1H), 8.55 (s, 1H), 7.82 (s, 1H), 7.62 (s, 1H), 4.82 (t, J=12.4 Hz, 1H), 4.54 (s, 1H), 4.10 (s, 1H), 2.38-2.48 (m, 1H), 2.10-2.35 (m, 4H), 1.40-1.70 (m, 6H), 1.20-1.38 (m, 2H), 1.23 (s, 3H), 0.85 (s, 3H).

### Example 8

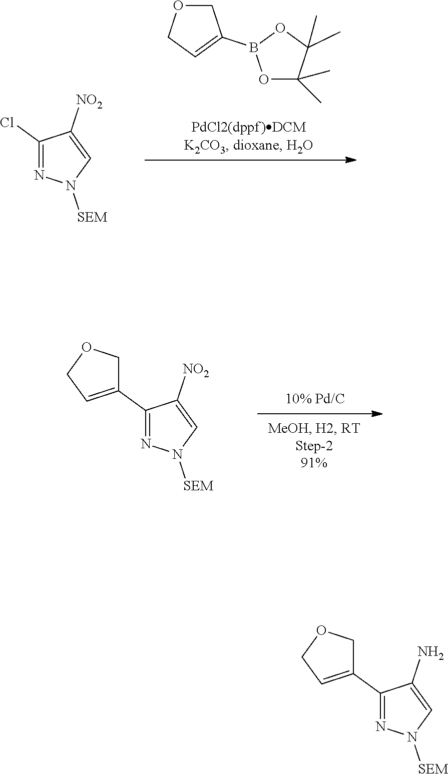 [SMILES: C[Si](C)(C)CCOCn1cc([N+](=O)[O-])c(Cl)n1 ; CC1(C)OB(C2=CCOC2)OC1(C)C ; C[Si](C)(C)CCOCn1cc([N+](=O)[O-])c(C2=CCOC2)n1 ; C[Si](C)(C)CCOCn1cc(N)c(C2=CCOC2)n1]

Procedure: To a degassed solution of 3-chloro-4-nitro-1-((2-(trimethylsilyl)ethoxy)methyl)-1H-pyrazole (0.8 g, 2.88 mmol) and 2-(2,5-dihydrofuran-3-yl)-4,4,5,5-tetramethyl-1,3,2-dioxaborolane (0.85 g, 4.32 mmol) in dioxane (6 mL) and H₂O (2 ml) was added K₂CO₃ (1 g, 7.20 mmol) and PdCl₂(dppf)·DCM (0.235 g 0.288 mmol), the reaction mass was closed under argon atmosphere and stirred at 100° C. for 16 h. The progress of the reaction was monitored by TLC and LCMS. After completion of the reaction, the reaction mass was filtered; the filtrate was concentrated under reduced pressure to give crude compound. The crude compound was purified by Sepa-Bean using silica gel (230-400 mesh), eluting with 0-20% ethyl acetate in pet-ether to give 3-(2,5-dihydrofuran-3-yl)-4-nitro-1-((2 (trimethylsilyl)ethoxy)methyl)-1H-pyrazole. (0.6 g, 60.7% yield) as yellow solid. LC-MS m/z: [M+H]+=312.35 ¹H NMR (400 MHz, CHLOROFORM-d) δ=8.30-8.15 (m, 1H), 7.15-7.06 (m, 1H), 5.46-5.36 (m, 2H), 5.06-4.84 (m, 4H), 3.73-3.58 (m, 2H), 1.01-0.82 (m, 2H), 0.07-−0.09 (m, 11H)

3-(tetrahydrofuran-3-yl)-1-((2-(trimethylsilyl)ethoxy)methyl)-1H-pyrazol-4-amine: To a parr-shaker containing 3-(2,5-dihydrofuran-3-yl)-4-nitro-1-((2 (trimethylsilyl)ethoxy)methyl)-1H-pyrazole (600 mg, 1.93 mmol) in methanol (6 mL), was added Pd/C (10%, 0.180 g) and stirred at room temperature at 80 psi hydrogen pressure for 2 h. The progress of the reaction was monitored by TLC. After completion of reaction, reaction mass was filtered through plug of celite, the filtrate was concentrated under reduced pressure to give the crude product 3-(tetrahydrofuran-3-yl)-1-((2-(trimethylsilyl)ethoxy)methyl)-1H-pyrazol-4-amine (0.5 g crude, 91% yield) as brown oil. LC-MS m/z: [M+H]+=284.48 ¹H NMR (400 MHz, DMSO-d₆) δ=7.12-6.96 (m, 1H), 5.17-5.08 (m, 2H), 4.03-3.35 (m, 12H), 2.21-1.96 (m, 3H), 1.79-1.71 (m, 1H), 0.86-0.70 (m, 3H), 0.06-−0.17 (m, 12H).

Coupling to Intermediate 1 and deprotection of SEM group to form 7′-((1R,3R)-3-hydroxycyclohexyl)-2′-((3-(tetrahydrofuran-3-yl)-1H-pyrazol-4-yl)amino)spiro[cyclopropane-1,5′-pyrrolo[2,3-d]pyrimidin]-6′(7′H)-one

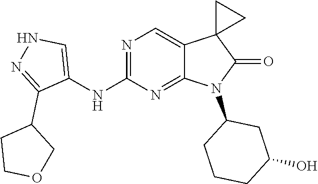 [SMILES: O=C1N([C@@H]2CCC[C@@H](O)C2)c2nc(Nc3c[nH]nc3C3CCOC3)ncc2C12CC2]

as per the method described in Example 2.

### Example 9

### Synthesis of

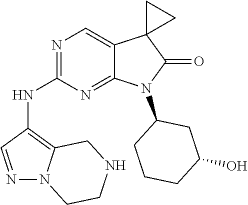 [SMILES: O=C1N([C@@H]2CCC[C@@H](O)C2)c2nc(Nc3cnn4c3CNCC4)ncc2C12CC2]

(7′-((1R,3R)-3-hydroxycyclohexyl)-2′-((4,5,6,7-tetrahydropyrazolo[1,5-a]pyrazin-3-yl)amino)spiro[cyclopropane-1,5′-pyrrolo[2,3-d]pyrimidin]-6′(7′H)-one).

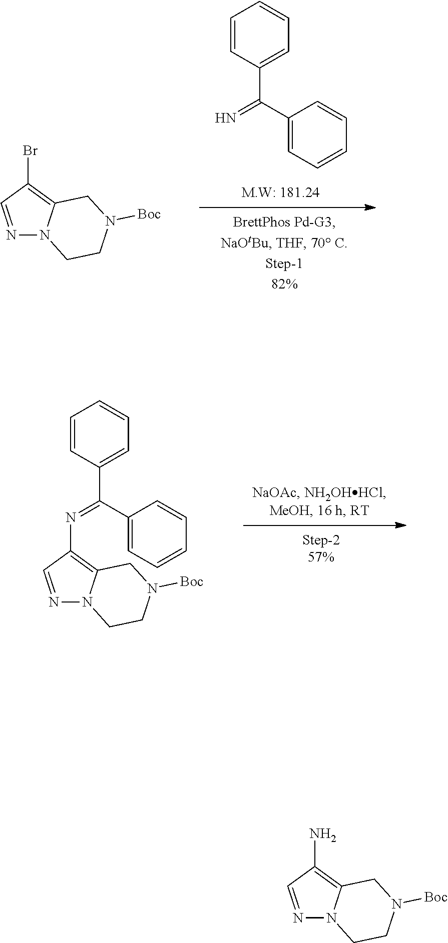 [SMILES: CC(C)(C)OC(=O)N1CCn2ncc(Br)c2C1 ; N=C(c1ccccc1)c1ccccc1 ; CC(C)(C)OC(=O)N1CCn2ncc(N=C(c3ccccc3)c3ccccc3)c2C1 ; CC(C)(C)OC(=O)N1CCn2ncc(N)c2C1]

### Step-1: Synthesis of tert-butyl 3-((diphenylmethylene)amino)-6,7-dihydropyrazolo[1,5-a]pyrazine-5(4H)-carboxylate (Compound-3)

Tert-butyl 3-((diphenylmethylene)amino)-6,7-dihydropyrazolo[1,5-a]pyrazine-5(4H)-carboxylate: To a stirred solution of tert-butyl 3-bromo-6,7-dihydropyrazolo[1,5-a]pyrazine-5(4H)-carboxylate (0.5 g, 1.65 mmol), diphenylmethanimine (0.447 g, 2.47 mmol) in THE (5 mL) was added NaOt-Bu (0.32 g, 3.31 mmol). The reaction mixture was purged by N₂ gas for 15 min and then was added BrettPhosPdG₃ (0.15 g, 0.165 mmol), purged for 2 min. The resulting reaction mixture was irradiated in microwave at 70° C. for 1 h. The progress of the reaction monitored by TLC and LCMS. After completion of reaction, reaction mass was passed through celite pad and the pad was washed with 10% MeOH in DCM (10 mL). The filtrate was washed with brine (10 mL), dried over Na₂SO₄ and concentrated under reduced pressure to give the crude product. The crude compound was purified by Sepa-Bean using silica gel (230-400 mesh), eluting with 0-30% ethyl acetate in pet-ether to give tert-butyl 3-((diphenylmethylene)amino)-6,7-dihydropyrazolo[1,5-a]pyrazine-5(4H)-carboxylate (0.6 g, 82% yield) as yellow solid. LC-MS m/z: [M+H]⁺=403.53 (91%)¹H NMR (400 MHz, CHLOROFORM-d) δ=7.76-7.74 (m, 2H), 7.50-7.22 (m, 8H), 6.02 (s, 1H), 4.79 (s, 2H), 4.07-3.8 (m, 4H), 1.57-1.49 (m, 9H).

Tert-butyl 3-amino-6,7-dihydropyrazolo[1,5-a]pyrazine-5(4H)-carboxylate: To a stirred solution of tert-butyl 3-((diphenylmethylene)amino)-6,7-dihydropyrazolo[1,5-a]pyrazine-5(4H)-carboxylate (0.6 g, 1.49 mmol) in methanol (6 mL), was added NaOAc (0.37 g, 4.47 mmol), followed by NH₂OH·HCl (0.42 g, 5.96 mmol) and the resulting reaction mixture was stirred at room temperature for 16 h. The progress of the reaction was monitored by TLC and LCMS. After completion of reaction, reaction mass was filtered and the filtrate was evaporated under reduced pressure to afford crude. The crude compound was purified by Sepa-Bean using silica gel (230-400 mesh), eluting with 0-10% MeOH in DCM to give tert-butyl 3-amino-6,7-dihydropyrazolo[1,5-a]pyrazine-5(4H)-carboxylate (0.27 g, 57% yield) as brown oil. LC-MS m/z: [M+H]⁺=239.13 ¹H NMR (400 MHz, DMSO-d₆) δ=6.920 (s, 1H), 4.41 (s, 2H), 3.94-3.92 (m, 2H), 3.78-3.75 (m, 2H), 3.17 (s, 2H), 1.906 (s, 3H), 1.44 (s, 9H).

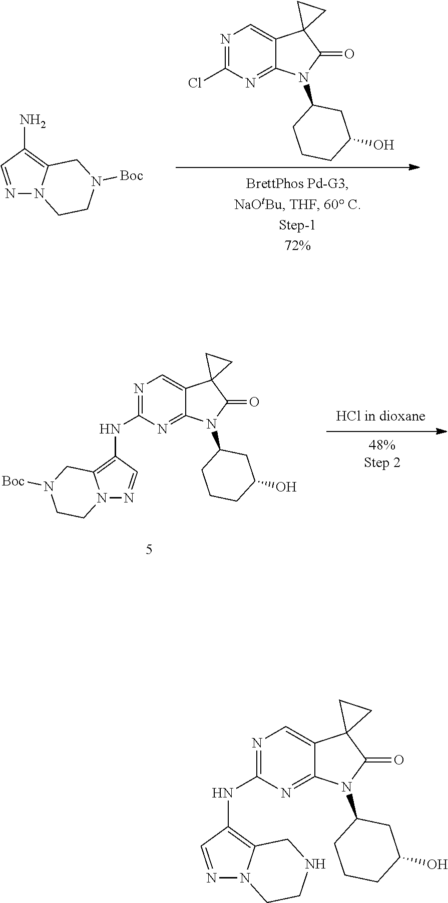 [SMILES: CC(C)(C)OC(=O)N1CCn2ncc(N)c2C1 ; O=C1N([C@@H]2CCC[C@@H](O)C2)c2nc(Cl)ncc2C12CC2 ; CC(C)(C)OC(=O)N1CCn2ncc(Nc3ncc4c(n3)N([C@@H]3CCC[C@@H](O)C3)C(=O)C43CC3)c2C1 ; O=C1N([C@@H]2CCC[C@@H](O)C2)c2nc(Nc3cnn4c3CNCC4)ncc2C12CC2]

Tert-butyl 3-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)-6,7-dihydropyrazolo[1,5-a]pyrazine-5(4H)-carboxylate: To a degassed solution of 2′-chloro-7′-((1R,3R)-3-hydroxycyclohexyl)spiro[cyclopropane-1,5′-pyrrolo[2,3-d]pyrimidin]-6′(7′H)-one (0.17 g, 0.578 mmol) and tert-butyl 3-amino-6,7-dihydropyrazolo[1,5-a]pyrazine-5(4H)-carboxylate (0.2 g, 0.868 mmol) in dry THE (2 mL), was added NaOt-Bu (85 mg, 0.868 mmol) and BrettphosPdG3 (53 mg, 0.057 mmol), the reaction mass was closed under argon atmosphere and irradiated in microwave at 60° C. for 1 h. The progress of the reaction was monitored by TLC and LCMS. After completion of the reaction, the reaction mass was filtered through celite pad; the filtrate was concentrated under reduced pressure to give crude compound. The crude compound was purified by Sepa-Bean using silica gel (230-400 mesh), eluting with 0-5% MeOH in DCM to give tert-butyl 3-((7′-((1R,3R)-3-hydroxycyclohexyl)-6′-oxo-6′,7-dihydrospiro[cyclopropane-1,5′-pyrrolo[2,3-d]pyrimidin]-2′-yl)amino)-6,7-dihydropyrazolo[1,5-a]pyrazine-5(4H)-carboxylate (0.2 g, 72% yield, 89% purity) as brown solid. LC-MS m/z: [M+H]⁺=496.36.

### 7′-((1R,3R)-3-hydroxycyclohexyl)-2′-((3-(tetrahydrofuran-3-yl)-1H-pyrazol-4-yl) amino) spiro[cyclopropane-1,5′-pyrrolo[2,3-d]pyrimidin]-6′(7′H)-one (SO-EE-PYZF)

To a stirred solution of trans-racemic-tert-butyl 3-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)-6,7-dihydropyrazolo[1,5-a]pyrazine-5(4H)-carboxylate (0.2 g, 0.4 mmol) in methanol (2 mL), was added 4M HCl in dioxane (2 mL) at 0° C. and stirred at RT for 2 h. Progress of the reaction was monitored by TLC & LCMS. After completion of reaction, reaction mass was diluted with ethyl acetate and concentrated under reduced pressure. The crude product was submitted for prep HPLC purification. COLUMN:—XB BUFFER A: ABC BUFFER B: ACETONITRILE, MOBILE PHASE CONDITIONS (% OF B):—0/10,2/10,10/30 FLOW-20ML ANL-MCL5;PREP-017 Diluent: ACN+THF+WATER Temperature:—Ambient The pure fractions obtained were evaporated under reduced pressure, lyophilized to give (78.7 mg, 48% yield) as white solid. LC-MS m/z: [M+H]⁺=396.22 (96.27%) ¹H NMR (400 MHz, DMSO-d₆) δ=8.707 (s, 1H), 7.80 (s, 1H), 7.53 (s, 1H), 4.75-4.58 (m, 2H), 4.12-4.03 (m, 1H), 3.92 (t, J=5.4 Hz, 2H), 3.85-3.76 (m, 2H), 3.13-3.01 (m, 2H), 2.46-2.30 (m, 1H), 2.29-2.13 (m, 1H), 1.763-1.516 (m, 10H).

### Example 10

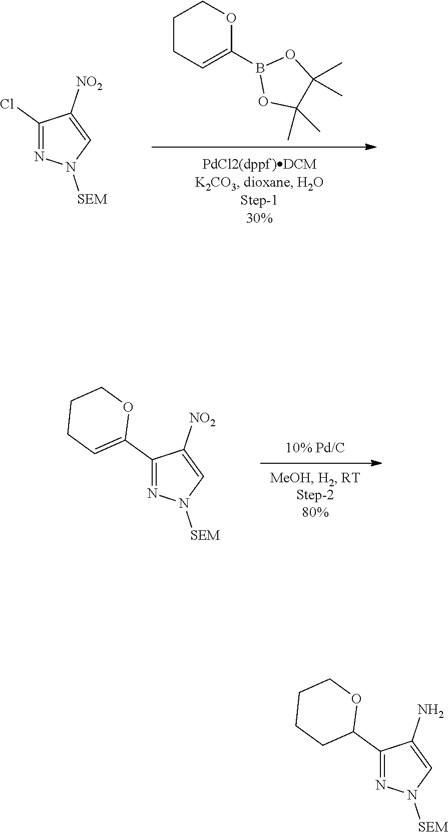 [SMILES: C[Si](C)(C)CCOCn1cc([N+](=O)[O-])c(Cl)n1 ; CC1(C)OB(C2=CCCCO2)OC1(C)C ; C[Si](C)(C)CCOCn1cc([N+](=O)[O-])c(C2=CCCCO2)n1 ; C[Si](C)(C)CCOCn1cc(N)c(C2CCCCO2)n1]

Procedure: To a degassed solution of 3-chloro-4-nitro-1-((2-(trimethylsilyl)ethoxy)methyl)-1H-pyrazole (1.0 g, 3.61 mmol) and 2-(3,4-dihydro-2H-pyran-6-yl)-4,4,5,5-tetramethyl-1,3,2-dioxaborolane (1.1 g, 5.41 mmol) in dioxane (6 mL) and H₂O (2 ml) was added K₂CO₃ (1.49 g, 10.83 mmol) and PdCl₂(dppf)·DCM (0.294 g 0.361 mmol), the reaction mass was closed under argon atmosphere and stirred at 100° C. for 16 h. The progress of the reaction was monitored by TLC and LCMS. After completion of the reaction, the reaction mass was filtered; the filtrate was concentrated under reduced pressure to give crude compound. The crude compound was purified by Sepa-Bean using silica gel (230-400 mesh), eluting with 0-20% ethyl acetate in pet-ether to give 3-(3,4-dihydro-2H-pyran-6-yl)-4-nitro-1-((2-(trimethylsilyl)ethoxy)methyl)-1H-pyrazole (0.36 g, 30% yield) as pale yellow oil. LC-MS m/z: [M+H]⁺=326.43

### Step-2: Synthesis of 3-(tetrahydro-2H-pyran-2-yl)-1-((2-(trimethylsilyl)ethoxy)methyl)-1H-pyrazol-4-amine (Compound-4)

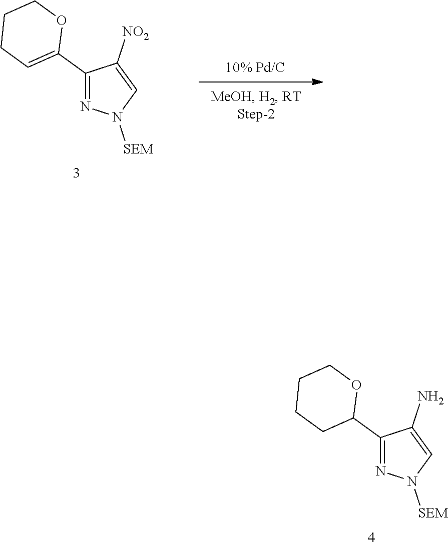 [SMILES: C[Si](C)(C)CCOCn1cc([N+](=O)[O-])c(C2=CCCCO2)n1 ; C[Si](C)(C)CCOCn1cc(N)c(C2CCCCO2)n1]

3-(tetrahydro-2H-pyran-2-yl)-1-((2-(trimethylsilyl)ethoxy)methyl)-1H-pyrazol-4-amine: To a parr-shaker containing 3-(3,4-dihydro-2H-pyran-6-yl)-4-nitro-1-((2-(trimethylsilyl)ethoxy)methyl)-1H-pyrazole (0.360 g, 1.107 mmol) in methanol (7 mL), was added Pd/C (10%, 0.15 g) and stirred at room temperature at 80 psi hydrogen pressure for 2 h. The progress of the reaction was monitored by TLC. After completion of reaction, reaction mass was filtered through plug of celite, the filtrate was concentrated under reduced pressure to give the crude product 3-(tetrahydro-2H-pyran-2-yl)-1-((2-(trimethylsilyl)ethoxy)methyl)-1H-pyrazol-4-amine (0.265 g crude, 80% yield) as pale yellow oil. LC-MS m/z: [M+H]⁺=298.19

Coupling to Intermediate 1 and deprotection of SEM group to form 7′-((1R,3R)-3-hydroxycyclohexyl)-2′-((3-(tetrahydro-2H-pyran-2-yl)-1H-pyrazol-4-yl)amino)spiro[cyclopropane-1,5′-pyrrolo[2,3-d]pyrimidin]-6′(7′H)-one

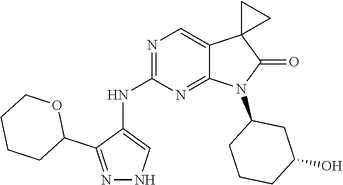 [SMILES: O=C1N([C@@H]2CCC[C@@H](O)C2)c2nc(Nc3c[nH]nc3C3CCCCO3)ncc2C12CC2]

as per the method described in Example 2.

### Example 11

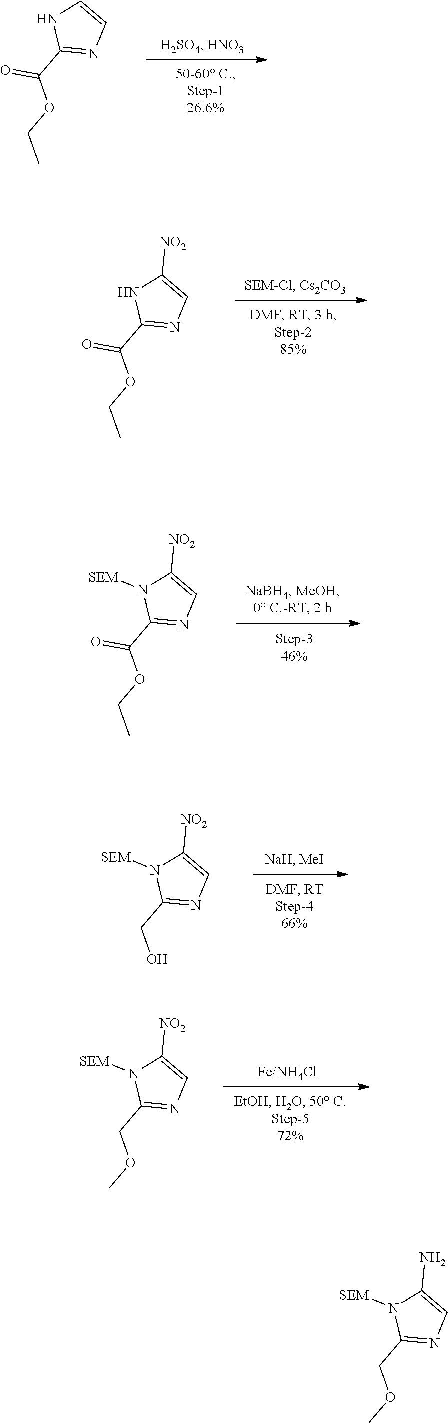

Ethyl 5-nitro-1H-imidazole-2-carboxylate: To a stirred solution of ethyl 1H-imidazole-2-carboxylate (8 g, 57.14 mmol) in H₂SO₄ (35 ml) was added HNO₃ (35 mL) dropwise at 0° C. and then stirred at 50-60° C. for 3 h. The progress of the reaction was monitored by TLC. After completion of reaction, reaction mass was quenched with ice-cold water and extracted with ethyl acetate (2×30 mL). The organic layer was washed with ice/water mixture, the precipitated crystals are collected by suction filtration and washed with 1500 ml of ice-water to give Ethyl 5-nitro-1H-imidazole-2-carboxylate (2.8 g, 26.6% yield) as white solid. LC-MS m/z: [M+H]⁺=186.27 (98%).

Ethyl 5-nitro-1-((2-(trimethylsilyl)ethoxy)methyl)-1H-imidazole-2-carboxylate: To a stirred solution of Ethyl 5-nitro-1H-imidazole-2-carboxylate (2.4 g, 12.96 mmol) and Cs₂CO₃ (8.42 g, 25.92 mmol) in DMF (24 mL), was added SEM-Cl (3.44 mL, 19.44 mmol) at 0° C. and then stirred at room temperature for 4 h. The progress of the reaction was monitored by TLC and LCMS. After completion of reaction, reaction mass was quenched with ice-cold water and extracted with ethyl acetate (2×200 mL). The organic layer was washed with ice-cold water (2×200 mL), dried over Na₂SO₄ and concentrated under reduced pressure to give the crude product. The crude compound was purified by Sepa-Bean using silica gel (230-400 mesh), eluting with 0-10% ethyl acetate in pet-ether to give Ethyl 5-nitro-1-((2-(trimethylsilyl)ethoxy)methyl)-1H-imidazole-2-carboxylate (3.5 g, 85% yield) as pale yellow liquid. ¹H NMR (400 MHz, DMSO-d₆) δ=8.046 (s, 1H), 5.827 (m, 2H), 4.482-4.428 (m, 2H), 3.665-3.623 (m, 2H), 2.945-2.872 (s, 1H), 1.448-1.413 (t, J=7.1 Hz, 3H), 0.993-0.951 (m, 2H), 0.013-−0.13 (s, 9H)

(5-nitro-1-((2-(trimethylsilyl)ethoxy)methyl)-1H-imidazol-2-yl)methanol: To a stirred solution of Ethyl 5-nitro-1-((2-(trimethylsilyl)ethoxy)methyl)-1H-imidazole-2-carboxylate (3.5 g, 11.09 mmol) in methanol (35 ml) was added NaBH₄ (0.839 g 22.194 mmol). Then stirred the reaction mixture for 2 h at RT, reaction progress monitored by TLC and LC-MS, after completion of reaction, reaction mass was quenched with sat. NH₄Cl solution and extracted with ethyl acetate (2×200 mL). The organic layer was washed with ice-cold water (2×200 mL), dried over Na₂SO₄ and concentrated under reduced pressure to give the crude product. The crude compound was purified by Sepa-Bean using silica gel (230-400 mesh), eluting with 10-30% ethyl acetate in pet-ether to give (5-nitro-1-((2-(trimethylsilyl)ethoxy)methyl)-1H-imidazol-2-yl)methanol (1.4 g, 46% yield) as pale yellow liquid. LC-MS m/z: [M+H]⁺=274.37 (89%)¹H NMR (400 MHz, CHLOROFORM-d) δ=7.863 (s, 1H), 5.592-5.458 (s, 2H), 4.855-4.765 (d, 2H), 3.633-3.592 (m, 2H), 2.793 (br s, 2H), 0.994-0.953 (m, 2H), 0.029 (m, 9H)

2-(methoxymethyl)-5-nitro-1-((2-(trimethylsilyl)ethoxy)methyl)-1H-imidazole: To a stirred solution of (5-nitro-1-((2-(trimethylsilyl)ethoxy)methyl)-1H-imidazol-2-yl)methanol (1.4 g, 5.12 mmol) in THE (14 mL), was added NaH (409 mg 10.24 mmol) and MeI (1.09 g 7.68 mmol) at 0° C. and stirred at RT for 1 h. The progress of the reaction was monitored by TLC & LCMS. After completion of reaction, reaction mass was quenched with sat. NH₄Cl solution and extracted with ethyl acetate (2×200 mL). The organic layer was washed with ice-cold water (2×200 mL), dried over Na₂SO₄ and concentrated under reduced pressure to give the crude product. The crude compound was purified by Sepa-Bean using silica gel (230-400 mesh), eluting with 10-20% ethyl acetate in pet-ether to give 2-(methoxymethyl)-5-nitro-1-((2-(trimethylsilyl)ethoxy)methyl)-1H-imidazole (0.630 g, 66% yield) as pale yellow liquid. LC-MS m/z: [M+H]⁺=288.23 (96%)¹H NMR (400 MHz, CHLOROFORM-d) δ=7.874 (s, 1H), 5.420 (s, 2H), 4.601 (s, 2H), 3.589-3.548 (m, 2H), 3.376 (s, 3H), 0.967-0.926 (m, 2H), 0.08-−0.006 (s, 9H)

2-(methoxymethyl)-1-((2-(trimethylsilyl)ethoxy)methyl)-1H-imidazol-5-amine: To a round-bottom flask containing 2-(methoxymethyl)-5-nitro-1-((2-(trimethylsilyl)ethoxy)methyl)-1H-imidazole (400 mg 1.393 mmol) in ethanol (4 ml) and water (4 ml), was added Fe (770.3 mg, 13.93 mmol) and NH₄Cl (749.4 mg 13.93 mmol) then stirred at 50° C. for 2 h. The progress of the reaction was monitored by TLC. The reaction mass was filtered through celite pad and washed with 10% MeOH in DCM (200 ml); the filtrate concentrated under reduced pressure to give the crude product, the crude product was diluted with DCM (50 ml) and filtered through glass funnel, the filtrate concentrated under reduced pressure to give 2-(methoxymethyl)-1-((2-(trimethylsilyl)ethoxy)methyl)-1H-imidazol-5-amine as brown colour oil (260 mg crude, 72% yield). The crude compound was directly used for next step LC-MS m/z: [M+H]⁺=258.53

Coupling to Intermediate 1 and deprotection of SEM group to form

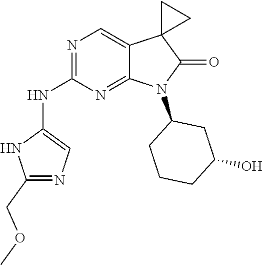 [SMILES: COCc1ncc(Nc2ncc3c(n2)N([C@@H]2CCC[C@@H](O)C2)C(=O)C32CC2)[nH]1]

(7′-((1R,3R)-3-hydroxycyclohexyl)-2′-((2-(methoxymethyl)-1H-imidazol-5-yl)amino)spiro[cyclopropane-1,5′-pyrrolo[2,3-d]pyrimidin]-6′(7′H)-one) as per the method described in Example 2.

### Example 12

Synthesis of

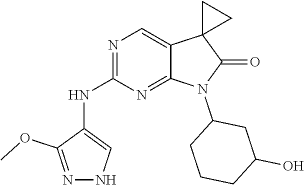 [SMILES: COc1n[nH]cc1Nc1ncc2c(n1)N(C1CCCC(O)C1)C(=O)C21CC1]

(7′-(3-hydroxycyclohexyl)-2′-((3-methoxy-1H-pyrazol-4-yl)amino)spiro[cyclopropane-1,5′-pyrrolo[2,3-d]pyrimidin]-6′(7′H)-one).

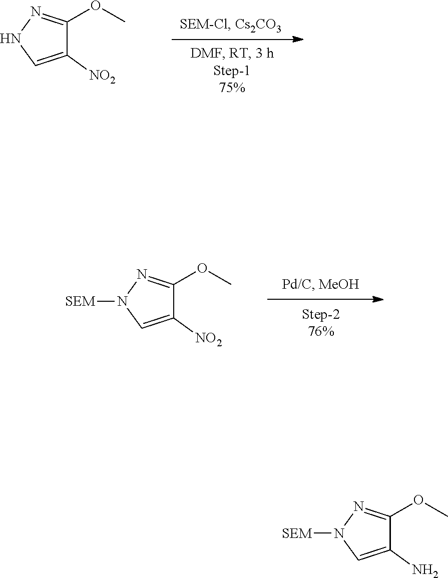

3-methoxy-4-nitro-1-((2-(trimethylsilyl)ethoxy)methyl)-1H-pyrazole: To a stirred solution of 3-methoxy-4-nitro-1H-pyrazole (5 g, 34.96 mmol) and Cs₂CO₃ (22.7 g, 69.92 mmol) in DMF (50 mL), was added SEM-Cl (9.3 mL, 52.44 mmol) at 0° C. and then stirred at room temperature for 3 h. The progress of the reaction was monitored by TLC and LCMS. After completion of reaction, reaction mass was quenched with ice-cold water and extracted with ethyl acetate (2×200 mL). The organic layer was washed with ice-cold water (2×500 mL), dried over Na₂SO₄ and concentrated under reduced pressure to give the crude product. The crude compound was purified by Sepa-Bean using silica gel (230-400 mesh), eluting with 0-10% ethyl acetate in pet-ether to give 3-methoxy-4-nitro-1-((2-(trimethylsilyl)ethoxy)methyl)-1H-pyrazole (7.2 g, 75% yield) as colourless liquid. LC-MS m/z: [M+H]⁺=274.37 (78%)¹H NMR (400 MHz, CHLOROFORM-d) δ=8.14 (s, 1H), 5.32 (s, 2H), 4.04 (s, 3H), 3.67-3.61 (m, 2H), 1.01-0.85 (m, 2H), 0.22-−0.18 (m, 9H).

3-methoxy-1-((2-(trimethylsilyl)ethoxy)methyl)-1H-pyrazol-4-amine: To a parr-shaker containing 3-methoxy-4-nitro-1-((2-(trimethylsilyl)ethoxy)methyl)-1H-pyrazole (7.2 g, 28.5 mmol) in methanol (72 mL), was added Pd/C (10%, 0.72 g) and stirred at room temperature at 80 psi hydrogen pressure for 1 h. The progress of the reaction was monitored by TLC. After completion of reaction, reaction mass was filtered through plug of celite and washed with 10% MeOH in DCM, the filtrates were concentrated under reduced pressure to give the crude product 3-methoxy-1-((2-(trimethylsilyl)ethoxy)methyl)-1H-pyrazol-4-amine (4.9 g, 76% yield) as brown liquid. LC-MS m/z: [M+H]⁺=243.21 (90%)¹H NMR (400 MHz, CHLOROFORM-d) δ=7.04 (s, 1H), 5.16-5.09 (m, 2H), 3.97-3.88 (m, 3H), 3.56-3.43 (m, 2H), 2.81-2.59 (m, 2H), 0.99-0.78 (m, 2H), 0.12-−0.05 (m, 9H).

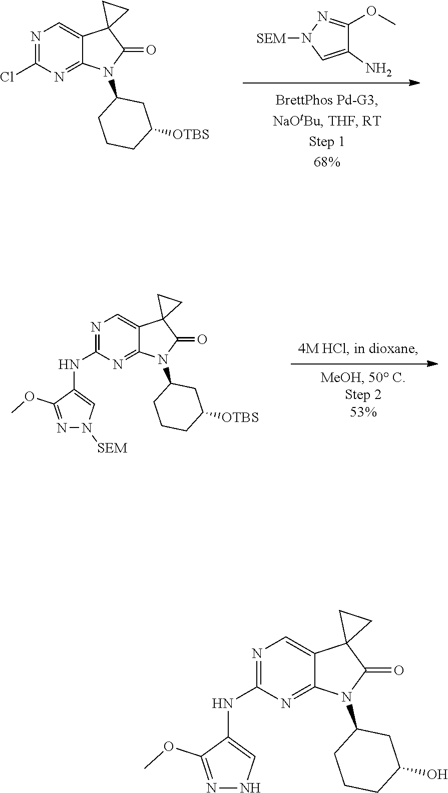 [SMILES: CC(C)(C)[Si](C)(C)O[C@@H]1CCC[C@@H](N2C(=O)C3(CC3)c3cnc(Cl)nc32)C1 ; COc1nn(COCC[Si](C)(C)C)cc1N ; COc1nn(COCC[Si](C)(C)C)cc1Nc1ncc2c(n1)N([C@@H]1CCC[C@@H](O[Si](C)(C)C(C)(C)C)C1)C(=O)C21CC1 ; COc1n[nH]cc1Nc1ncc2c(n1)N([C@@H]1CCC[C@@H](O)C1)C(=O)C21CC1]

Step 1: 7′-((1R,3R)-3-((tert-butyldimethylsilyl)oxy)cyclohexyl)-2′-((3-methoxy-1-((2-(trimethylsilyl)ethoxy)methyl)-1H-pyrazol-4-yl)amino)spiro[cyclopropane-1,5′-pyrrolo[2,3-d]pyrimidin]-6′(7′H)-one: To a degassed solution of 7′-((1R,3R)-3-((tert-butyldimethylsilyl)oxy)cyclohexyl)-2′-((3-methoxy-1-((2-(trimethylsilyl)ethoxy)methyl)-1H-pyrazol-4-yl)amino)spiro[cyclopropane-1,5′-pyrrolo[2,3-d]pyrimidin]-6′(7′H)-one (10 g, 24.5 mmol) and 3-methoxy-1-((2-(trimethylsilyl)ethoxy)methyl)-1H-pyrazol-4-amine (8.9 g, 36.7 mmol) in dry THE (200 mL), was added NaOt-Bu (3.5 g, 36.75 mmol) and BrettphosPdG3 (2.21 g, 2.45 mmol), the reaction mass was closed under argon atmosphere and stirred at room temperature for 4 h. The progress of the reaction was monitored by TLC and LCMS. After completion of the reaction, the reaction mass was filtered; the filtrate was concentrated under reduced pressure to give crude compound. The crude compound was purified by Sepa-Bean using silica gel (230-400 mesh), eluting with 0-30% ethyl acetate in pet-ether to give 7′-((1R,3R)-3-((tert-butyldimethylsilyl)oxy)cyclohexyl)-2′-((3-methoxy-1-((2-(trimethylsilyl)ethoxy)methyl)-1H-pyrazol-4-yl)amino)spiro[cyclopropane-1,5′-pyrrolo[2,3-d]pyrimidin]-6′(7′H)-one. The obtained was dissolved in DCM and stirred with Si-Thiourea (Palladium scavenger) for 3 h and filtered through celite pad. The filtrate was concentrated under reduced pressure to give 7′-((1R,3R)-3-((tert-butyldimethylsilyl)oxy)cyclohexyl)-2′-((3-methoxy-1-((2-(trimethylsilyl)ethoxy)methyl)-1H-pyrazol-4-yl)amino)spiro[cyclopropane-1,5′-pyrrolo[2,3-d]pyrimidin]-6′(7′H)-one (10.2 g, 68% yield) as brown gummy solid. LC-MS m/z: [M+H]⁺=615.43; ¹H NMR (400 MHz, CHLOROFORM-d) δ=7.97 (s, 1H), 7.63 (s, 1H), 6.65-6.53 (m, 1H), 5.40-5.11 (m, 2H), 4.05-3.98 (m, 3H), 3.64-3.49 (m, 2H), 2.67-2.25 (m, 1H), 1.89-1.21 (m, 9H), 1.03-0.78 (m, 9H), 0.02-−0.05 (m, 9H).

Step-2: 7′-((1R,3R)-3-hydroxycyclohexyl)-2′-((3-methoxy-1H-pyrazol-4-yl)amino)spiro[cyclopropane-1,5′-pyrrolo[2,3-d]pyrimidin]-6′(7′H)-one: To a stirred solution of 7-((1R,3R)-3-((tert-butyldimethylsilyl)oxy)cyclohexyl)-2′-((3-methoxy-1-((2-(trimethylsilyl)ethoxy)methyl)-1H-pyrazol-4-yl)amino)spiro[cyclopropane-1,5′-pyrrolo[2,3-d]pyrimidin]-6′(7′H)-one (10.2 g, 16.5 mmol) in methanol (102 mL), was added 4M HCl in dioxane (153 mL) at 0° C. and stirred at 50° C. for 16 h. The progress of the reaction was monitored by TLC & LCMS. After completion of reaction, reaction mass was diluted with ethyl acetate and concentrated under reduced pressure. The crude product was basified with aq. NaHCO₃ and extracted with ethyl acetate (3×200 mL). The organic layer was dried over Na₂SO₄, concentrated under reduced pressure to get the crude compound. The crude product was purified by trituration with 5% MeOH in DCM to give 7′-((1R,3R)-3-hydroxycyclohexyl)-2′-((3-methoxy-1H-pyrazol-4-yl)amino)spiro[cyclopropane-1,5′-pyrrolo[2,3-d]pyrimidin]-6′(7′H)-one (3.3 g, 53% yield) as off white solid.

LC-MS m/z: [M+H]⁺=371.38, ¹H NMR (400 MHz, DMSO-d₆) δ=11.71-11.51 (s, 1H), 8.19 (s, 1H), 7.78 (s, 1H), 7.647-7.643 (d, 1H), 4.649-4.619 (m, 1H), 4.52 (d, J=2.8 Hz, 1H), 4.06 (br s, 1H), 3.78 (s, 3H), 2.35-2.10 (m, 1H), 1.83-1.19 (m, 10H).

### Example 13

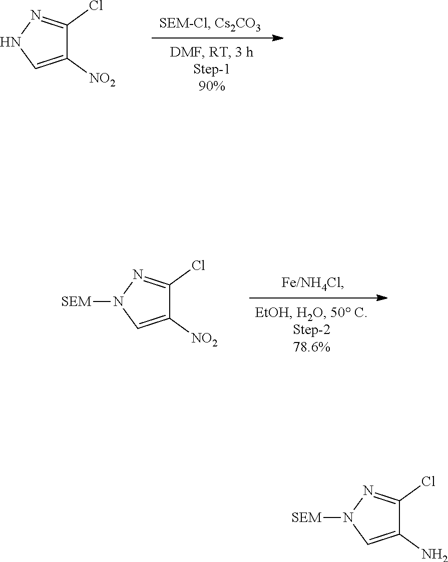 [SMILES: O=[N+]([O-])c1c[nH]nc1Cl ; C[Si](C)(C)CCOCn1cc([N+](=O)[O-])c(Cl)n1 ; C[Si](C)(C)CCOCn1cc(N)c(Cl)n1]

3-chloro-4-nitro-1-((2-(trimethylsilyl)ethoxy)methyl)-1H-pyrazole: To a stirred solution of 3-chloro-4-nitro-1H-pyrazole (3.0 g 20.4 mmol) and Cs₂CO₃ (13.26 g, 40.8 mmol) in DMF (30 ml), was added SEM-Cl (5.4 ml 30.61 mmol) at 0° C. and then stirred at room temperature for 2 h. The progress of the reaction was monitored by TLC and LCMS. The reaction was quenched with ice-cold water (100 mL) and extracted with ethyl acetate (2×200 mL). The organic layer was washed with ice-cold water (2×100 mL), dried over Na₂SO₄ and concentrated under reduced pressure to give the crude product. The crude compound was purified by Sepa-Bean using silica gel (230-400 mesh), eluting with 0-10% ethyl acetate in pet-ether to give 3-chloro-4-nitro-1-((2-(trimethylsilyl)ethoxy)methyl)-1H-pyrazole as pale yellow oil (5.2 g, 90% yield). ¹H NMR (400 MHz, CHLOROFORM-d) δ=8.33-8.19 (m, 1H), 5.53 (s, 1H), 5.52-5.39 (m, 2H), 3.67-3.63 (m, 2H), 0.97-0.91 (m, 2H), 0.02-−0.01 (m, 9H)

3-chloro-1-((2-(trimethylsilyl)ethoxy)methyl)-1H-pyrazol-4-amine: To a stirred solution of 3-chloro-4-nitro-1-((2-(trimethylsilyl)ethoxy)methyl)-1H-pyrazole (2.5 g 9.02 mmol) in ethanol (10V) and water (10V) was added NH₄Cl (4.78 g, 90.25 mmol) followed by Fe (5.03 g, 90.25 mmol) and the reaction mixture was stirred at 50° C. for 1 h. The progress of the reaction was monitored by TLC. The reaction mass was filtered through plug of celite and washed with 10% MeOH in DCM; the filtrate was washed with water (50 mL) and extracted with 10% MeOH in DCM concentrated under reduced pressure to give 3-chloro-1-((2-(trimethylsilyl)ethoxy)methyl)-1H-pyrazol-4-amine as brown gummy solid (2.0 g, 78.66% yield). LC-MS m/z: [M+H]⁺=248.11 (87%)¹H NMR (400 MHz, DMSO-d₆) δ=7.40-7.20 (m, 1H), 5.31-5.27 (m, 2H), 5.22 (s, 1H), 4.14 (br s, 2H), 3.54-3.48 (m, 2H), 0.91-0.82 (m, 2H), 0.08-−0.13 (m, 9H)

Coupling to Intermediate 1 and deprotection of SEM group to form 2′-((3-chloro-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

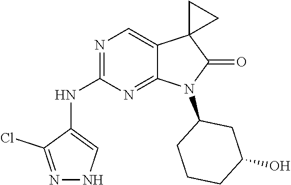 [SMILES: O=C1N([C@@H]2CCC[C@@H](O)C2)c2nc(Nc3c[nH]nc3Cl)ncc2C12CC2]

as per the method described in Example 2.

### Example 14

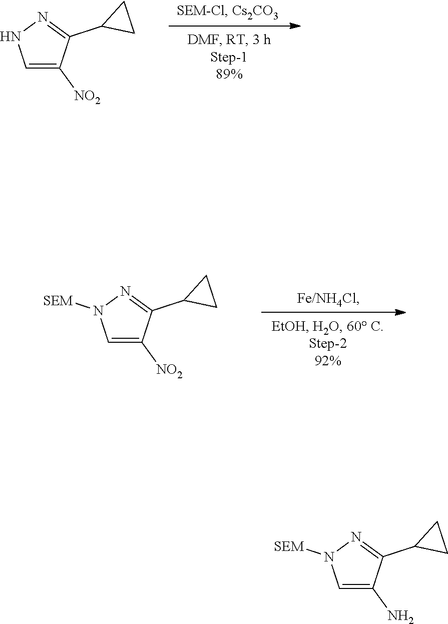 [SMILES: O=[N+]([O-])c1c[nH]nc1C1CC1 ; C[Si](C)(C)CCOCn1cc([N+](=O)[O-])c(C2CC2)n1 ; C[Si](C)(C)CCOCn1cc(N)c(C2CC2)n1]

3-cyclopropyl-4-nitro-1-((2-(trimethylsilyl)ethoxy)methyl)-1H-pyrazole: To a stirred solution of 3-cyclopropyl-4-nitro-1H-pyrazole (3.0 g 19.6 mmol) and Cs₂CO₃ (12.74 g 39.2 mmol) in DMF (30 ml), was added SEM-Cl (5.1 ml 29.4 mmol) at 0° C. and then stirred at room temperature for 2 h. The progress of the reaction was monitored by TLC and LCMS. The reaction was quenched with ice-cold water and extracted with ethyl acetate (2×200 mL). The organic layer was washed with ice-cold water (2×100 mL), dried over Na₂SO₄ and concentrated under reduced pressure to give the crude product. The crude compound was purified by Sepa-Bean using silica gel (230-400 mesh), eluting with 0-10% ethyl acetate in pet-ether to give 3-cyclopropyl-4-nitro-1-((2-(trimethylsilyl)ethoxy)methyl)-1H-pyrazole as colourless liquid (4.9 g, 89% yield). LC-MS m/z: was not ionizing, ¹H NMR (400 MHz, CDCl3, 298 K) δ (ppm)=8.23-8.06 (m, 1H), 5.55-5.30 (m, 2H), 3.68-3.57 (m, 2H), 2.64-2.02 (m, 1H), 1.24-1.03 (m, 4H), 1.02-0.92 (m, 2H), 0.05 (s, 9H)

3-cyclopropyl-1-((2-(trimethylsilyl)ethoxy)methyl)-1H-pyrazol-4-amine: To a Parr-shaker containing 3-cyclopropyl-4-nitro-1-((2-(trimethylsilyl)ethoxy)methyl)-1H-pyrazole (2.0 g 7.067 mmol) in Ethanol (20 ml) and water (20 ml), was added Fe (1.97 g 35.3 mmol) and NH₄Cl (1.88 g 35.3 mmol) then stirred at 60° C. for 2 h. The progress of the reaction was monitored by TLC. The reaction mass was filtered through celite pad and washed with 10% MeOH in DCM (200 ml); the filtrate was washed with water (50 ml) and extracted with 10% MeOH in DCM The organic layers was concentrated under reduced pressure to give the crude product 3-cyclopropyl-1-((2-(trimethylsilyl)ethoxy)methyl)-1H-pyrazol-4-amine as brown colour oil (1.65 g, 92% yield). The crude compound was directly used for next step LC-MS m/z: [M+H]⁺=254.21 ¹H NMR (400 MHz, DMSO-d₆) δ=7.12-6.93 (m, 1H), 5.35-5.15 (m, 2H), 4.072 (brs, 2H), 3.52-3.41 (m, 2H), 1.78-1.61 (m, 1H), 0.82-0.75 (m, 4H), 0.67-0.64 (m, 2H), 0.01-−0.03 (s, 9H).

Coupling to Intermediate 1 and deprotection of SEM group to form 2′-((3-cyclopropyl-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

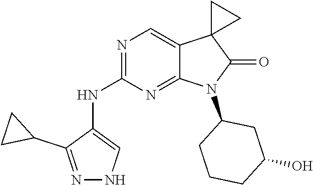 [SMILES: O=C1N([C@@H]2CCC[C@@H](O)C2)c2nc(Nc3c[nH]nc3C3CC3)ncc2C12CC2]

as per the method described in Example 2.

### Example 15

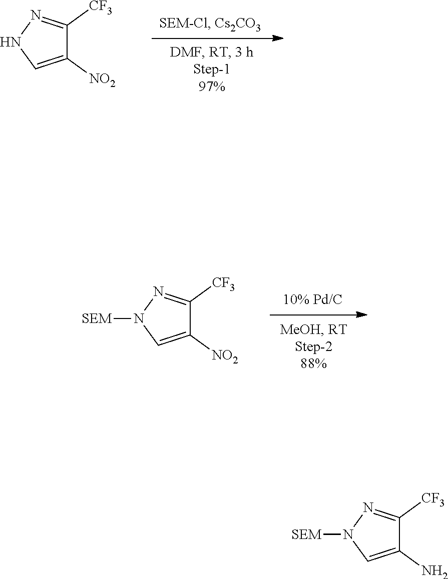 [SMILES: O=[N+]([O-])c1c[nH]nc1C(F)(F)F ; C[Si](C)(C)CCOCn1cc([N+](=O)[O-])c(C(F)(F)F)n1 ; C[Si](C)(C)CCOCn1cc(N)c(C(F)(F)F)n1]

4-nitro-3-(trifluoromethyl)-1-((2-(trimethylsilyl)ethoxy)methyl)-1H-pyrazole: To a stirred solution of 4-nitro-3-(trifluoromethyl)-1H-pyrazole (3.0 g 16.56 mmol) and Cs₂CO₃ (10.7 g 33.13 mmol) in DMF (30 ml), was added SEM-Cl (4.4 ml 24.85 mmol) at 0° C. and then stirred at room temperature for 3 h. The progress of the reaction was monitored by TLC and LCMS. The reaction was quenched with ice-cold water and extracted with ethyl acetate (2×200 mL). The organic layer was washed with ice-cold water (2×100 mL), dried over Na₂SO₄ and concentrated under reduced pressure to give the crude product. The crude compound was purified by Sepa-Bean using silica gel (230-400 mesh), eluting with 0-10% ethyl acetate in pet-ether to give 4-nitro-3-(trifluoromethyl)-1-((2-(trimethylsilyl)ethoxy)methyl)-1H-pyrazole as a colour less liquid (5.0 g, 97% yield). LC-MS m/z: was not ionizing, ¹H NMR (400 MHz, DMSO-d₆) δ=9.36-8.61 (m, 1H), 5.74-5.56 (m, 2H), 3.65-3.61 (m, 2H), 0.89-0.84 (2H), 0.05-0.01 (m, 9H)

3-(trifluoromethyl)-1-((2-(trimethylsilyl)ethoxy)methyl)-1H-pyrazol-4-amine: To a Parr-shaker containing 4-nitro-3-(trifluoromethyl)-1-((2-(trimethylsilyl)ethoxy)methyl)-1H-pyrazole (3.0 g 9.64 mmol) in methanol (30 mL), was added Pd/C (10%, 0.3 g) and the reaction mixture was stirred at room temperature with 80 psi hydrogen pressure for 2 h. The progress of the reaction was monitored by TLC. The reaction mass was filtered through celite pad and washed with 10% MeOH in DCM (200 mL); the filtrate was concentrated under reduced pressure to give the crude product 3-(trifluoromethyl)-1-((2-(trimethylsilyl)ethoxy)methyl)-1H-pyrazol-4-amine as a pale yellow liquid (2.4 g (crude), 88% yield). This crude compound was directly used for next step. LC-MS m/z: [M+H]⁺=282.18 (92%), ¹H NMR (400 MHz, DMSO-d₆) δ=7.33-7.14 (m, 1H), 5.32-5.29 (m, 2H), 4.69-4.31 (m, 2H), 3.54-3.42 (m, 2H), 0.83-0.79 (m, 2H), 0.0-−0.065 (m, 9H).

Coupling to Intermediate 1 and deprotection of SEM group to form 7′-((1R,3R)-3-hydroxycyclohexyl)-2′-((3-(trifluoromethyl)-1H-pyrazol-4-yl)amino)spiro[cyclopropane-1,5′-pyrrolo[2,3-d]pyrimidin]-6′(7′H)-one

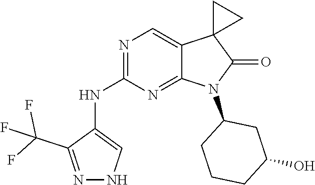 [SMILES: O=C1N([C@@H]2CCC[C@@H](O)C2)c2nc(Nc3c[nH]nc3C(F)(F)F)ncc2C12CC2]

as per the method described in Example 2.

### Example 16

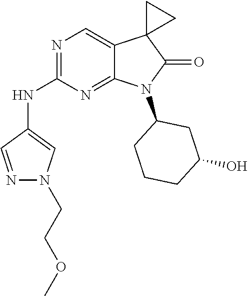 [SMILES: COCCn1cc(Nc2ncc3c(n2)N([C@@H]2CCC[C@@H](O)C2)C(=O)C32CC2)cn1]

(7′-((1R,3R)-3-hydroxycyclohexyl)-2′-((1-(2-methoxyethyl)-1H-pyrazol-4-yl)amino)spiro[cyclopropane-1,5′-pyrrolo[2,3-d]pyrimidin]-6′(7′H)-one) was made using the method of Example 1, using

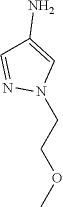

(commercially available) instead of

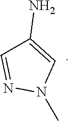

### Example 17

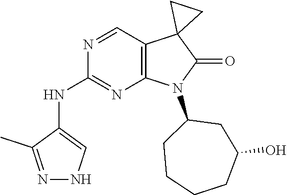 [SMILES: Cc1n[nH]cc1Nc1ncc2c(n1)N([C@@H]1CCCC[C@@H](O)C1)C(=O)C21CC1]

(7′-(3-hydroxycycloheptyl)-2′-((3-methyl-1H-pyrazol-4-yl)amino)spiro[cyclopropane-1,5′-pyrrolo[2,3-d]pyrimidin]-6′(7′H)-one) was made using the method of Example 2, but using Intermediate 2 rather than Intermediate 1.

### Example 17 Alternative Synthesis

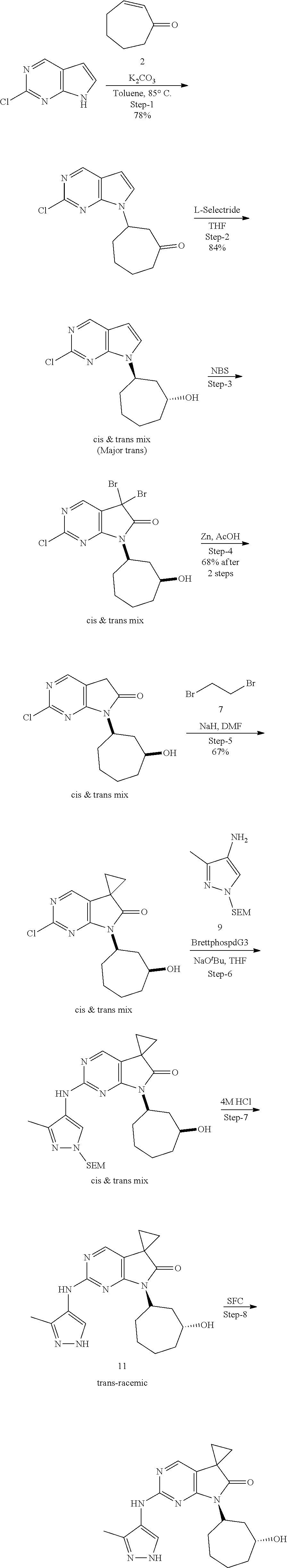

3-(2-chloro-7H-pyrrolo[2,3-d]pyrimidin-7-yl)cycloheptan-1-one: To a stirred solution of 2-chloro-7H-pyrrolo[2,3-d]pyrimidine (10 g, 65.11 mmol) and K₂CO₃ (13.5 g, 97.69 mmol) in toluene (100 ml), was added cyclohept-2-en-1-one (21.4 g, 195.40 mmol) and heated at 8500 for 16 h. The progress of the reaction was monitored by TLC and LCMS. After completion of the reaction, the reaction mass was filtered through plug of celite, the filtrate was concentrated under reduced pressure to give the crude product. The crude compound was purified by column chromatography using silica gel (100-200 mesh), eluting with 0-40% ethyl acetate in pet-ether to give 3-(2-chloro-7H-pyrrolo[2,3-d]pyrimidin-7-yl)cycloheptan-1-one (13.3 g, 78%) as pale yellow solid. LC-MS m/z [M+H]+=264.31, ¹H NMR (400 MHz, CDCl₃) δ=8.79 (m, 1H), 7.23 (d, J=3.6 Hz, 1H), 6.59 (d, J=3.6 Hz, 1H), 5.05-5.15 (m, 1H), 3.22 (dd, J=11.2, 15.0 Hz, 1H), 2.85-2.92 (m, 1H), 2.62-2.72 (m, 1H), 2.02-2.30 (m, 4H), 1.70-1.82 (m, 2H).

3-(2-chloro-7H-pyrrolo[2,3-d]pyrimidin-7-yl)cycloheptan-1-ol (racemate, diasteromeric mixture): To a stirred solution of 3-(2-chloro-7H-pyrrolo[2,3-d]pyrimidin-7-yl)cycloheptan-1-one (10 g, 38.02 mmol) in dry THE (100 mL), was added L-selectride (57 mL, 57.03 mmol) at −78° C. and stirred at same temperature for 2 h. The progress of the reaction was monitored by TLC & LCMS. After completion of the reaction, the reaction mass was quenched with sat. NH₄Cl (50 mL) and extracted with ethyl acetate (3×50 mL). The combined organic layers were dried over Na₂SO₄, concentrated under reduced pressure. The crude compound was purified by column chromatography using silica gel (100-200 mesh), eluting with 0-50% ethyl acetate in pet-ether to give 3-(2-chloro-7H-pyrrolo[2,3-d]pyrimidin-7-yl)cycloheptan-1-ol (64% trans) & (15% cis) (8.5 g, 84% yield as mixture of cis-trans isomers) as pale yellow solid. LC-MS m/z [M+H]+=266.39

5,5-dibromo-2-chloro-7-(3-hydroxycycloheptyl)-5,7-dihydro-6H-pyrrolo[2,3-d]pyrimidin-6-one (racemate, diasteromeric mixture): To a stirred solution of 3-(2-chloro-7H-pyrrolo[2,3-d]pyrimidin-7-yl)cycloheptan-1-ol (8.5 g, 23.28 mmol) in tert-butanol (85 mL) and water (17 mL), was added NBS (12.4 g, 69.86 mmol) at room temperature and stirred for 3 h. Progress of the reaction was monitored by TLC and LCMS. After completion of the reaction, the reaction mass was diluted with water (50 mL) and extracted with ethyl acetate (3×75 mL), the combined organic layers were dried over Na₂SO₄ and concentrated under reduced pressure to give crude 5,5-dibromo-2-chloro-7-(3-hydroxycycloheptyl)-5,7-dihydro-6H-pyrrolo[2,3-d]pyrimidin-6-one (14.5 g crude, mixture of cis-trans isomers) as brown gummy liquid. The crude compound used as such for next step without further purification.

2-chloro-7-(3-hydroxycycloheptyl)-5,7-dihydro-6H-pyrrolo[2,3-d]pyrimidin-6-one (racemate, diasteromeric mixture): To a stirred solution of 5,5-dibromo-2-chloro-7-(3-hydroxycycloheptyl)-5,7-dihydro-6H-pyrrolo[2,3-d]pyrimidin-6-one (14 g crude, 31.85 mmol) in AcOH (140 mL) was added Zinc-dust (10 g, 159.27 mmol) at room temperature and stirred for 1 h. Progress of the reaction was monitored by TLC and LCMS. After completion of the reaction, the reaction mass was filtered through celite-pad, filtrate was concentrated under reduced pressure. The crude was diluted with ethyl acetate (300 mL) and washed with water (2×50 mL). The organic layer was dried over Na₂SO₄, concentrated under reduced pressure. The crude compound was purified by column chromatography using silica gel (230-400 mesh), eluting with 0-50% ethyl acetate in pet-ether to give 2-chloro-7-(3-hydroxycycloheptyl)-5,7-dihydro-6H-pyrrolo[2,3-d]pyrimidin-6-one (77% cis) and (10% trans) (4.5 g, 68% yield after two steps) as brown colour solid. LC-MS m/z [M+H]+=282.33

2′-chloro-7′-(3-hydroxycycloheptyl)spiro[cyclopropane-1,5′-pyrrolo[2,3-d]pyrimidin]-6′(7′H)-one (Racemate, diastereomeric mixture): To a stirred solution of 2-chloro-7-(3-hydroxycycloheptyl)-5,7-dihydro-6H-pyrrolo[2,3-d]pyrimidin-6-one (3 g, 10.64 mmol) and dibromoethane (4.6 mL, 53.24 mmol) in dry DMF (30 mL), was added NaH (60%, 2.1 g, 53.24 mmol) portion wise at room temperature and stirred for 3 h. Progress of the reaction was monitored by TLC & LCMS. After completion of the reaction, the reaction was quenched with ice-cold water (10 mL), extracted with ethyl acetate (2×30 mL). The organic layer was washed with ice-cold water (2×10 mL), dried over Na₂SO₄, concentrated under reduced pressure to get the crude product. The crude product was purified by column chromatography (silica gel, 230-400 mesh), eluting with 0-70% ethyl acetate in pet ether to give 2′-chloro-7′-(3-hydroxycycloheptyl)spiro[cyclopropane-1,5′-pyrrolo[2,3-d]pyrimidin]-6′(7′H)-one (79% trans) & (13% cis) (2.2 g, 67% yield) as pale brown solid. LC-MS m/z [M+H]⁺=308.21, ¹H NMR (400 MHz, CDCl₃) δ=7.78-7.84 (m, 1H), 4.79-4.89 (m, 1H), 4.29 (s, 1H), 2.62-2.72 (m, 1H), 2.27-2.42 (m, 1H), 1.93-2.08 (m, 2H), 1.70-1.90 (m, 7H), 1.55-1.70 (m, 3H), 1.42-1.58 (m, 1H).

7′-(3-hydroxycycloheptyl)-2′-((3-methyl-1-((2-(trimethylsilyl)ethoxy)methyl)-1H-pyrazol-4-yl)amino)spiro[cyclopropane-1,5′-pyrrolo[2,3-d]pyrimidin]-6′(7′H)-one (Racemate, diastereomeric mixture): To a degassed solution of 2′-chloro-7′-(3-hydroxycycloheptyl)spiro[cyclopropane-1,5′-pyrrolo[2,3-d]pyrimidin]-6′(7′H)-one (0.35 g, 1.14 mmol) and 3-methyl-1-((2-(trimethylsilyl)ethoxy)methyl)-1H-pyrazol-4-amine (0.31 g, 1.36 mmol) in dry THE (4 mL), was added NaOtBu (0.16 g, 1.71 mmol) and BrettphosPdG3 (0.10 g, 0.11 mmol), the reaction mass was closed under argon atmosphere and stirred at room temperature for 2 h. Progress of the reaction was monitored by TLC and LCMS. After completion of the reaction, the reaction mass was filtered; the filtrate was concentrated under reduced pressure to give crude compound. The crude compound was purified by Sepa-Bean using silica gel (230-400 mesh), eluting with 0-70% ethyl acetate in pet-ether to give 7′-(3-hydroxycycloheptyl)-2′-((3-methyl-1-((2-(trimethylsilyl)ethoxy)methyl)-1H-pyrazol-4-yl)amino)spiro[cyclopropane-1,5′-pyrrolo[2,3-d]pyrimidin]-6′(7′H)-one (0.35 g, 53% yield) as pale yellow liquid. LC-MS m/z [M+H]⁺=499.67 & 499.57.

7′-(3-hydroxycycloheptyl)-2′-((3-methyl-1H-pyrazol-4-yl)amino)spiro[cyclopropane-1,5′-pyrrolo[2,3-d]pyrimidin]-6′(7′H)-one (trans-racemic): To a stirred solution of 7′-(3-hydroxycycloheptyl)-2′-((3-methyl-1-((2-(trimethylsilyl)ethoxy)methyl)-1H-pyrazol-4-yl)amino)spiro[cyclopropane-1,5′-pyrrolo[2,3-d]pyrimidin]-6′(7′H)-one (0.3 g, 0.60 mmol) in methanol (1.5 mL), was added 4M HCl in dioxane (1.5 mL) at 0° C. and stirred at 50° C. for 3 h. Progress of the reaction was monitored by TLC & LCMS. After completion of reaction, reaction mass was concentrated under reduced pressure. The crude product was basified with aq. NaHCO₃ and extracted with 5% MeOH in DCM (3×15 mL). The organic layer was dried over Na₂SO₄, concentrated under reduced pressure to get the crude compound. The crude product was purified by Prep. HPLC (Column: YMC trait C₁₈ Buffer A: 10 mm ABC Buffer B:—ACN solubility:—ACN-THF-water mobile phase conditions (% of B):—0/35,2/50,12/60,12.01/100,16/100,16.01/35,20/35 flow-20/min) to give 7′-(3-hydroxycycloheptyl)-2′-((3-methyl-1H-pyrazol-4-yl)amino)spiro[cyclopropane-1,5′-pyrrolo[2,3-d]pyrimidin]-6′(7′H)-one (peak-2, 40 mg, trans-isomer) as off white solid. LC-MS m/z [M+H]+=369.43, ¹H NMR (400 MHz, DMSO-d₆) δ=11.50-12.80 (br-s, 1H), 8.54 (br-s, 1H), 7.80 (s, 1H), 7.69 (s, 1H), 4.68-4.80 (m, 1H), 4.49 (s, 1H), 3.98 (s, 1H), 2.50-2.62 (m, 1H), 2.10-2.20 (m, 3H), 2.20-2.28 (m, 1H), 1.60-1.80 (m, 6H), 1.55-1.64 (m, 3H), 1.40-1.54 (m, 3H), 1.30-1.40 (m, 1H).

7′-((1R,3R)-3-hydroxycycloheptyl)-2′-((3-methyl-1H-pyrazol-4-yl)amino)spiro[cyclopropane-1,5′-pyrrolo[2,3-d]pyrimidin]-6′(7′H)-one: Trans-racemic 7-(3-hydroxycycloheptyl)-2′-((3-methyl-1H-pyrazol-4-yl)amino)spiro[cyclopropane-1,5′-pyrrolo[2,3-d]pyrimidin]-6′(7′H)-one (30 mg) was subjected to SFC separation (Chiralpak IC, 65% CO₂, 35% (30 mM methanolic ammonia in methanol as co-solvent). After completion of the separation, the solvent of peak-1 was distilled off to give 7′-((1R,3R)-3-hydroxycycloheptyl)-2′-((3-methyl-1H-pyrazol-4-yl)amino)spiro[cyclopropane-1,5′-pyrrolo[2,3-d]pyrimidin]-6′(7′H)-one (0.136 g, 38%) as white solid. LC-MS m/z [M+H]+=369.40, ¹H NMR (400 MHz, DMSO-d₆) δ=12.09 (br-s, 1H), 8.54 (s, 1H), 7.80 (s, 1H), 7.69 (s, 1H), 4.68-4.78 (m, 1H), 4.49 (s, 1H), 3.98 (s, 1H), 2.50-2.60 (m, 1H), 2.10-2.25 (m, 4H), 1.63-1.82 (m, 5H), 1.56-1.62 (m, 3H), 1.41-1.55 (m, 3H), 1.30-1.50 (m, 1H).

### Example 18

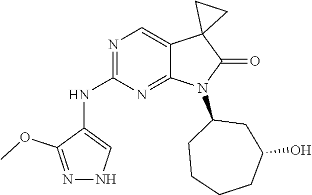 [SMILES: COc1n[nH]cc1Nc1ncc2c(n1)N([C@@H]1CCCC[C@@H](O)C1)C(=O)C21CC1]

(7′-((1R,3R)-3-hydroxycycloheptyl)-2′-((3-methoxy-1H-pyrazol-4-yl)amino)spiro[cyclopropane-1,5′-pyrrolo[2,3-d]pyrimidin]-6′(7′H)-one) was made using the method of Example 12, but using Intermediate 2 rather than Intermediate 1.

Alternatively, the route of Example 17 alternative synthesis can be used, but using

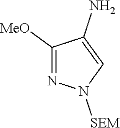 [SMILES: COc1nn(COCC[Si](C)(C)C)cc1N]

instead of

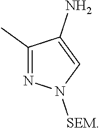

### Example 19

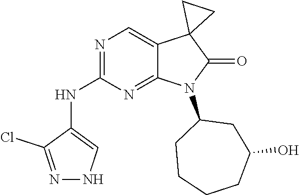 [SMILES: O=C1N([C@@H]2CCCC[C@@H](O)C2)c2nc(Nc3c[nH]nc3Cl)ncc2C12CC2]

(2′-((3-chloro-1H-pyrazol-4-yl)amino)-7′-((1R,3R)-3-hydroxycycloheptyl)spiro[cyclopropane-1,5′-pyrrolo[2,3-d]pyrimidin]-6′(7′H)-one) was made using the method of Example 13, but using Intermediate 2 rather than Intermediate 1.

Alternatively, the route of Example 17 alternative synthesis can be used, but using

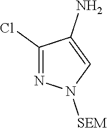 [SMILES: C[Si](C)(C)CCOCn1cc(N)c(Cl)n1]

instead of

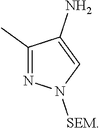

### Example 20

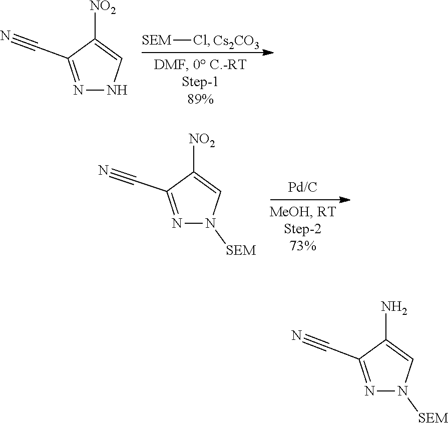

4-nitro-1-((2-(trimethylsilyl)ethoxy)methyl)-1H-pyrazole-3-carbonitrile: To a stirred solution of 4-nitro-1H-pyrazole-3-carbonitrile (3 g, 21.70 mmol) and Cs₂CO₃ (14.1 g, 43.40 mmol) in DMF (150 mL), was added SEM-Cl (5.7 mL, 32.60 mmol) at 0° C. and then stirred at room temperature for 3 h. The progress of the reaction was monitored by TLC. After completion of the reaction, the reaction was quenched with ice-cold water and extracted with ethyl acetate (2×50 mL). The organic layer was washed with ice-cold water (2×10 mL), dried over Na₂SO₄ and concentrated under reduced pressure to give the crude product. The crude compound was purified by Sepa-Bean using silica gel (230-400 mesh), eluting with 0-10% ethyl acetate in pet-ether to give 4-nitro-1-((2-(trimethylsilyl)ethoxy)methyl)-1H-pyrazole-3-carbonitrile as pale yellow liquid (5.2 g, 89% yield). ¹H NMR (400 MHz, CDCl₃) δ=8.40 (s, 1H), 5.52 (s, 2H), 3.62-3.72 (m, 2H), 0.92-1.0 (m, 2H), 0.00-0.05 (m, 9H).

4-amino-1-((2-(trimethylsilyl)ethoxy)methyl)-1H-pyrazole-3-carbonitrile: To a Parr-shaker containing 4-nitro-1-((2-(trimethylsilyl)ethoxy)methyl)-1H-pyrazole-3-carbonitrile (2 g, 7.46 mmol) in methanol (20 mL), was added Pd/C (0.2 g) and the reaction mass was stirred at room temperature with 80 psi hydrogen pressure for 2 h. Progress of the reaction was monitored by TLC. After completion of the reaction, the reaction mass was filtered through plug of celite, the filtrate was concentrated under reduced pressure to give the crude product. The crude compound was purified by Sepa-Bean using silica gel (230-400 mesh), eluting with 0-20% ethyl acetate in pet-ether to give 4-amino-1-((2-(trimethylsilyl)ethoxy)methyl)-1H-pyrazole-3-carbonitrile as pale brown sloid (1.3 g, 73% yield). LC-MS m/z [M+H]⁺=239.48, ¹H NMR (400 MHz, DMSO-d₆) δ=7.35 (s, 1H), 5.31 (s, 2H), 4.82 (s, 2H), 4.50 (t, J=8.0 Hz, 2H), 0.82 (t, J=8.0 Hz, 2H), −0.08-0.0 (m, 9H).

Coupling to Intermediate 1 and deprotection of SEM group to form 4-((7′-((1R,3R)-3-hydroxycyclohexyl)-6′-oxo-6′,7-dihydrospiro[cyclopropane-1,5′-pyrrolo[2,3-d]pyrimidin]-2′-yl)amino)-1H-pyrazole-3-carbonitrile

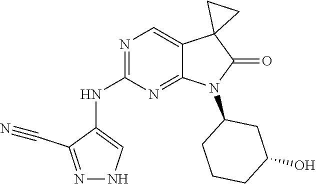 [SMILES: N#Cc1n[nH]cc1Nc1ncc2c(n1)N([C@@H]1CCC[C@@H](O)C1)C(=O)C21CC1]

as per the method described in Example 2.

### Example 21

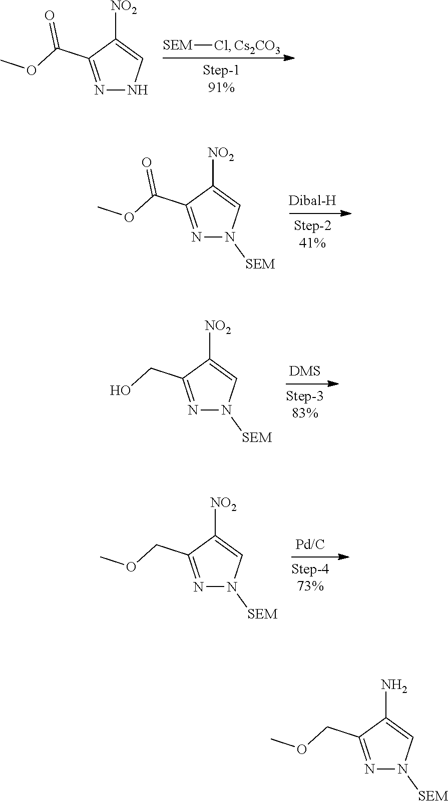

Methyl 4-nitro-1-((2-(trimethylsilyl)ethoxy)methyl)-1H-pyrazole-3-carboxylate: To a stirred solution of methyl 4-nitro-1H-pyrazole-3-carboxylate (2 g, 11.69 mmol) and Cs₂CO₃ (7.6 g, 23.39 mmol) in DMF (20 mL), was added SEM-Cl (3.2 mL, 17.54 mmol) at 0° C. and then stirred at room temperature for 2 h. The progress of the reaction was monitored by TLC and LCMS. After completion of reaction, reaction mass was quenched with ice-cold water and extracted with ethyl acetate (2×30 mL). The organic layer was washed with ice-cold water (2×10 mL), dried over Na₂SO₄ and concentrated under reduced pressure to give the crude product. The crude compound was purified by Sepa-Bean using silica gel (230-400 mesh), eluting with 0-30% ethyl acetate in pet-ether to give methyl 4-nitro-1-((2-(trimethylsilyl)ethoxy)methyl)-1H-pyrazole-3-carboxylate (3.2 g, 91% yield) as colourless liquid. ¹H NMR (400 MHz, DMSO-d₆) δ=9.18 (s, 1H), 8.46 (s, 1H), 5.60 (s, 2H), 5.51 (s, 2H), 3.97 (s, 3H), 3.89 (s, 3H), 3.50-3.64 (m, 4H), 0.80-0.90 (m, 4H), −0.05-0.01 (m, 9H).

(4-nitro-1-((2-(trimethylsilyl)ethoxy)methyl)-1H-pyrazol-3-yl)methanol: To a stirred solution of methyl 4-nitro-1-((2-(trimethylsilyl)ethoxy)methyl)-1H-pyrazole-3-carboxylate (1.2 g, 3.98 mmol) in dry THE (12 ml) was added Dibal-H (12 ml, 15.80 mmol) drop wise at −30° C., then reaction mass was raised to room temperature and stirred for 3 h. The progress of the reaction was monitored TLC & LCMS. After completion of the reaction, quenched with 1N HCl and extracted with ethyl acetate (3×30 mL). The organic layer was dried over Na₂SO₄, concentrated to give crude compound. The crude compound was purified by Sepa-Bean using silica gel (230-400 mesh), eluting with 0-50% ethyl acetate in pet-ether to give (4-nitro-1-((2-(trimethylsilyl)ethoxy)methyl)-1H-pyrazol-3-yl)methanol (0.45 g, 41% yield) as pale orange liquid. ¹H NMR (400 MHz, CDCl₃) δ=8.32 (s, 1H), 5.42 (s, 2H), 4.93 (d, J=6.8 Hz, 2H), 3.60-3.68 (m, 2H), 2.82 (t, J=6.8 Hz, 1H), 0.92-0.98 (m, 2H), −0.06-0.05 (m, 9H).

3-(methoxymethyl)-4-nitro-1-((2-(trimethylsilyl)ethoxy)methyl)-1H-pyrazole: To a stirred solution of (4-nitro-1-((2-(trimethylsilyl)ethoxy)methyl)-1H-pyrazol-3-yl)methanol (0.4 g, 1.46 mmol) in dry THE (15 ml) was added NaH (60%, 0.09 g, 3.65 mmol) at 0° C. and stirred for 30 min. After, dimethyl sulfate (0.16 mL, 1.75 mmol) was added and stirred at 50° C. for 4 h. The progress of the reaction was monitored TLC & LCMS.

After completion of the reaction, quenched with water and extracted with ethyl acetate (3×30 mL). The organic layer was dried over Na₂SO₄, concentrated to give crude compound. The crude compound was purified by Sepa-Bean using silica gel (230-400 mesh), eluting with 0-30% ethyl acetate in pet-ether to give 3-(methoxymethyl)-4-nitro-1-((2-(trimethylsilyl)ethoxy)methyl)-1H-pyrazole (0.35 g, 83% yield) as pale brown liquid. ¹H NMR (400 MHz, CDCl₃) δ=8.33 (s, 1H), 5.44 (s, 2H), 4.81 (s, 2H), 3.64 (t, J=8.0 Hz, 2H), 3.51 (s, 3H), 0.94 (t, J=8.0 Hz, 2H), −0.09-0.02 (m, 9H).

3-(methoxymethyl)-1-((2-(trimethylsilyl)ethoxy)methyl)-1H-pyrazol-4-amine: To a stirring solution of 3-(methoxymethyl)-4-nitro-1-((2-(trimethylsilyl)ethoxy)methyl)-1H-pyrazole (0.35 g, 1.21 mmol) in methanol (6 mL), was added Pd/C (10%, 0.1 g) and stirred at room temperature under hydrogen balloon pressure for 2 h. Progress of the reaction was monitored by TLC. After completion of reaction, reaction mass was filtered through plug of celite, the filtrate was concentrated under reduced pressure to give the crude 3-(methoxymethyl)-1-((2-(trimethylsilyl)ethoxy)methyl)-1H-pyrazol-4-amine (0.23 g, 73% yield) as pale green liquid. The crude product used as such without further purification. ¹H NMR (400 MHz, CDCl₃) δ=7.14 (s, 1H), 5.29 (s, 2H), 4.55 (s, 2H), 3.53 (t, J=8.0 Hz, 2H), 3.92 (s, 3H), 0.92 (t, J=8.0 Hz, 2H), −0.01-0.08 (m, 9H).

Coupling to Intermediate 1 and deprotection of SEM group to form 7′-((1R,3R)-3-hydroxycyclohexyl)-2′-((3-(methoxymethyl)-1H-pyrazol-4-yl)amino)spiro[cyclopropane-1,5′-pyrrolo[2,3-d]pyrimidin]-6′(7′H)-one

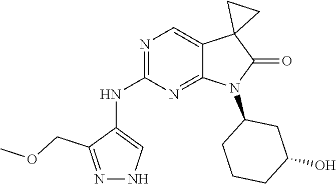 [SMILES: COCc1n[nH]cc1Nc1ncc2c(n1)N([C@@H]1CCC[C@@H](O)C1)C(=O)C21CC1]

as per the method described in Example 2.

### Example 22

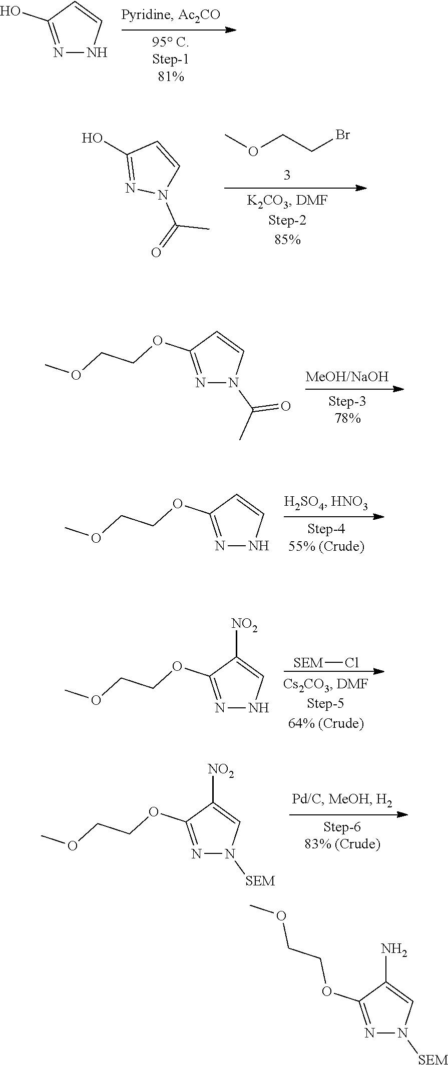

1-(3-hydroxy-1H-pyrazol-1-yl)ethan-1-one: To a stirred solution of 1H-pyrazol-3-ol (5 g, 59.5 mmol) in pyridine (26 mL) was added Ac₂O (5.9 ml, 62.05 mmol) in 9 mL of pyridine at 95° C. over 30 min. The reaction mass was stirred under argon atmosphere at 95° C. for 3.5 hs. Progress of the reaction was monitored by TLC and LCMS. After completion of the reaction, the reaction mass was concentrated under reduced pressure to give crude compound. The crude compound was purified by trituration with methanol (15 mL) and filtered through Buchner funnel and washed with methanol and dried to give 1-(3-hydroxy-1H-pyrazol-1-yl)ethan-1-one (6.1 g, 81% yield) as off white solid. LC-MS m/z: [M+H]⁺=127.00 ¹H NMR (400 MHz, DMSO-d₆) δ=10.956 (s, 1H), 8.132-8.125 (d, J=2.8 HZ, 1H), 6.012-6.005 (d, J=2.8 HZ, 1H), 2.507 (s, 3H).

1-(3-(2-methoxyethoxy)-1H-pyrazol-1-yl)ethan-1-one: To a degassed solution of 1-(3-hydroxy-1H-pyrazol-1-yl)ethan-1-one (2 g, 15.87 mmol) and 1-bromo-2-methoxyethane (2.28 g, 16.66 mmol) in DMF (20 mL), was added K₂CO₃ (3.28 g, 23.8 mmol), the reaction mass was stirred under argon atmosphere at 85° C. for 2 h. Progress of the reaction was monitored by TLC and LCMS. After completion of the reaction, the reaction mass was quenched with ice-cold water and extracted with ethyl acetate (2×200 mL). The organic layer was washed with ice-cold water (2×500 mL), dried over Na₂SO₄ and concentrated under reduced pressure to give the crude product. The crude compound was purified by Sepa-Bean using silica gel (230-400 mesh), eluting with 10-30% ethyl acetate in pet-ether to give 1-(3-(2-methoxyethoxy)-1H-pyrazol-1-yl)ethan-1-one (2.5 g, 85% yield) as pale yellow liquid. LC-MS m/z: [M+H]+=185.31 ¹H NMR (400 MHz, CDCl3) δ=8.054-8.046 (d, J=3.3 HZ, 1H), 6.007-6.000 (d, J=3.3 HZ, 1H), 4.414 (m, 2H), 3.757-3.727 (m, 2H), 3.44 (s, 3H), 2.594 (s, 3H).

3-(2-methoxyethoxy)-1H-pyrazole: To a degassed solution of 1-(3-(2-methoxyethoxy)-1H-pyrazol-1-yl)ethan-1-one (2.5 g, 13.58 mmol) in MeOH (17.5 ml), was added 8M NaOH (6.8 ml, 135.8 mmol) solution, the reaction mass was closed under argon atmosphere and stirred at 50° C. for 1 h. Progress of the reaction was monitored by TLC and LCMS. After completion of the reaction, the reaction mass was quenched with ice-cold water and extracted with ethyl acetate (2×200 mL). The organic layer was washed with ice-cold water (2×500 mL), dried over Na₂SO₄ and concentrated under reduced pressure to give the crude 3-(2-methoxyethoxy)-1H-pyrazole (1.5 g crude, 78% yield) as pale yellow solid. LC-MS m/z: [M+H]⁺=143.21 (82% purity)¹H NMR (400 MHz, CDCL3) δ=9.169 (br s, 1H), 7.357-7.351 (d, J=2.4 HZ, 1H), 5.772-5.766 (d, J=2.4 HZ, 1H), 4.344-4.321 (m, 2H), 3.752-3.728 (m, 2H), 3.441 (s, 3H).

3-(2-methoxyethoxy)-4-nitro-1H-pyrazole: To a solution of 3-(2-methoxyethoxy)-1H-pyrazole (1.5 g, 10.56 mmol) in H₂SO₄ (6 mL, 4 V), was added HNO₃ (1.5 mL, 1V), and the reaction mass was stirred under argon atmosphere at 50° C. for 3 h. Progress of the reaction was monitored by TLC and LCMS. After completion of the reaction, the reaction mass was quenched with ice-cold water and extracted with ethyl acetate (2×200 mL). The organic layer was washed with ice-cold water (2×500 mL), dried over Na₂SO₄ and concentrated under reduced pressure to give the crude 3-(2-methoxyethoxy)-4-nitro-1H-pyrazole (1.1 g crude, 55% yield) as yellow solid. LC-MS m/z: [M+H]⁺=188.32 (76%)

3-(2-methoxyethoxy)-4-nitro-1-((2-(trimethylsilyl)ethoxy)methyl)-1H-pyrazole: To a stirred solution of 3-(2-methoxyethoxy)-4-nitro-1H-pyrazole (1.1 g, 5.88 mmol) and Cs₂CO₃ (3.44 g, 10.58 mmol) in DMF (11 mL), was added SEM-Cl (1.55 mL, 8.82 mmol) at 0° C. and then stirred at room temperature for 3 h. The progress of the reaction was monitored by TLC and LCMS. After completion of reaction, reaction mass was quenched with ice-cold water and extracted with ethyl acetate (2×200 mL). The organic layer was washed with ice-cold water (2×500 mL), dried over Na₂SO₄ and concentrated under reduced pressure to give the crude product. The crude compound was purified by Sepa-Bean using silica gel (230-400 mesh), eluting with 10-40% ethyl acetate in pet-ether to give 3-(2-methoxyethoxy)-4-nitro-1-((2-(trimethylsilyl)ethoxy)methyl)-1H-pyrazole (1.2 g, 64% yield) as Pale yellow colour liquid. LC-MS m/z: [M+H]⁺=318.48

3-(2-methoxyethoxy)-1-((2-(trimethylsilyl)ethoxy)methyl)-1H-pyrazol-4-amine: To a parr-shaker containing 3-(2-methoxyethoxy)-4-nitro-1-((2-(trimethylsilyl)ethoxy)methyl)-1H-pyrazole (1.2 g, 3.78 mmol) in methanol (24 mL), was added Pd/C (10%, 120 mg) and stirred at room temperature at 80 psi hydrogen pressure for 1 h. Progress of the reaction was monitored by TLC. After completion of reaction, reaction mass was filtered through celite pad and washed with 10% MeOH in DCM, the filtrates were concentrated under reduced pressure to give the crude product 3-(2-methoxyethoxy)-1-((2-(trimethylsilyl)ethoxy)methyl)-1H-pyrazol-4-amine (900 mg crude, 83% yield) as brown liquid. LC-MS m/z: [M+H]⁺=288.08

Coupling to Intermediate 1 and deprotection of SEM group to form 7′-((1R,3R)-3-hydroxycyclohexyl)-2′-((3-(2-methoxyethoxy)-1H-pyrazol-4-yl)amino)spiro[cyclopropane-1,5′-pyrrolo[2,3-d]pyrimidin]-6′(7′H)-one

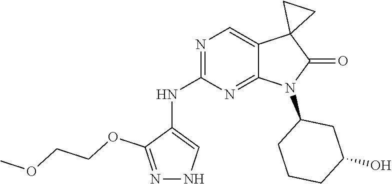 [SMILES: COCCOc1n[nH]cc1Nc1ncc2c(n1)N([C@@H]1CCC[C@@H](O)C1)C(=O)C21CC1]

as per the method described in Example 2.

### Example 23

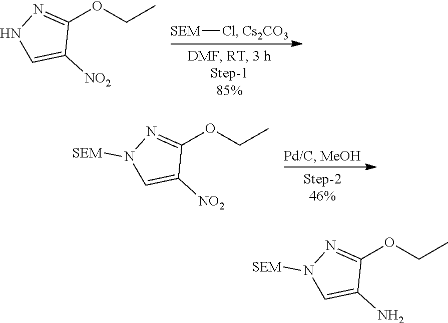

3-ethoxy-4-nitro-1-((2-(trimethylsilyl)ethoxy)methyl)-1H-pyrazole: To a stirred solution of 3-ethoxy-4-nitro-1H-pyrazole (900 mg, 5.73 mmol) and Cs₂CO₃ (3.7 g, 11.46 mmol) in DMF (9 mL), was added SEM-Cl (1.51 mL, 8.59 mmol) at 0° C. and then stirred at room temperature for 4 h. The progress of the reaction was monitored by TLC and LCMS. After completion of reaction, reaction mass was quenched with ice-cold water and extracted with ethyl acetate (2×200 mL). The organic layer was washed with ice-cold water (2×500 mL), dried over Na₂SO₄ and concentrated under reduced pressure to give the crude product. The crude compound was purified by Sepa-Bean using silica gel (230-400 mesh), eluting with 0-10% ethyl acetate in pet-ether to give 3-ethoxy-4-nitro-1-((2-(trimethylsilyl)ethoxy)methyl)-1H-pyrazole (1.4 g, 85% yield) as Pale yellow colour liquid.

LC-MS m/z: not ionizing (78%)¹H NMR (400 MHz, CHLOROFORM-d) δ=8.136 (s, 1H), 5.245 (s, 2H), 4.410-4.392 (m, 2H), 3.365-3.609 (m, 2H), 1.485-1.450 (t, J=6.8 HZ, 3H), 0.961-0.919 (m, 2H), 0.036-0.011 (s, 9H).

3-ethoxy-1-((2-(trimethylsilyl)ethoxy)methyl)-1H-pyrazol-4-amine: To a parr-shaker containing 3-ethoxy-4-nitro-1-((2-(trimethylsilyl)ethoxy)methyl)-1H-pyrazole (500 g, 1.74 mmol) in methanol (5 mL), was added Pd/C (10%, 50 mg) and stirred at room temperature at 80 psi hydrogen pressure for 1 h. Progress of the reaction was monitored by TLC. After completion of reaction, reaction mass was filtered through celite pad and washed with 10% MeOH in DCM, the filtrates were concentrated under reduced pressure to give the crude product 3-ethoxy-1-((2-(trimethylsilyl)ethoxy)methyl)-1H-pyrazol-4-amine (210 mg, 46% yield) as brown liquid. LC-MS m/z: [M+H]⁺=258.45

Coupling to Intermediate 1 and deprotection of SEM group to form 2′-((3-ethoxy-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

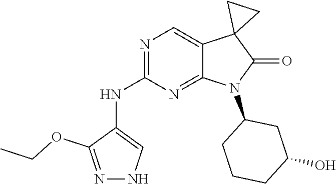 [SMILES: CCOc1n[nH]cc1Nc1ncc2c(n1)N([C@@H]1CCC[C@@H](O)C1)C(=O)C21CC1]

as per the method described in Example 2.

### Example 24

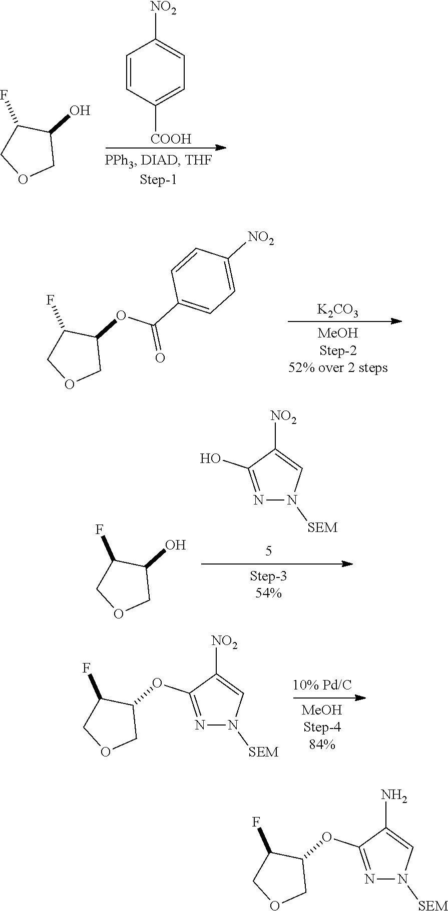

(Cis racemate)-4-fluorotetrahydrofuran-3-yl 4-nitrobenzoate: To a stirred solution of PPh₃ (11.53 g, 35.34 mmol) and DIAD (7.1 g, 35.34 mmol) in THE (100 mL) at RT was added a mixture of (Trans-racemate)-4-fluorotetrahydrofuran-3-ol (2.5 g, 23.56 mmol) and 4-nitrobenzoic acid (5.90 g, 35.34 mmol) dissolved in THE (10 mL). The reaction mixture was stirred at RT for 16 h. The progress of the reaction was monitored by TLC and LCMS. After completion of the reaction, the reaction mixture was evaporated under vacuum to afford crude compound. The crude compound was purified by Sepa-Bean using silica gel (100-200 mesh), by eluting with 15-20% ethyl acetate in pet-ether and evaporated to give (Cis-racemate)-4-fluorotetrahydrofuran-3-yl 4-nitrobenzoate (5.0 g, 83% yield) as an Off-white solid. ¹H NMR (400 MHz, CDCl₃) δ=8.29-8.33 (m, 2H), 8.24-8.27 (m, 2H), 4.80-5.10 (m, 2H), 4.01-4.25 (m, 4H).

(Cis racemate)-4-fluorotetrahydrofuran-3-ol: To a stirred solution of (Cis racemate)-4-fluorotetrahydrofuran-3-yl 4-nitrobenzoate (5.0 g, 19.59 mmol) in MeOH (250 mL) at RT was added K₂CO₃ (13.54 g, 97.96 mmol). The reaction mixture was stirred at RT under argon atmosphere for 2 h. The progress of the reaction was monitored by TLC. After completion of the reaction, the reaction mixture was filtered and filtrate was evaporated under reduced pressure to give crude compound. The crude compound was purified by Sepa-Bean using silica gel (100-200 mesh), eluting with 50% ethyl acetate in pet-ether to give (Cis-racemate)-4-fluorotetrahydrofuran-3-ol. (1.3 g, 52% yield over 2 steps) as a Pale brown syrup. ¹H NMR (400 MHz, CDCl₃) δ=4.95-5.10 (m, 1H), 4.28-4.41 (m, 1H), 3.95-4.11 (m, 4H), 2.237 (br-s, 1H).

(Trans-racemate) 3-((-4-fluorotetrahydrofuran-3-yl) oxy)-4-nitro-1-((2-(trimethylsilyl) ethoxy) methyl)-1H-pyrazole: To a stirring solution of 4-nitro-1-((2-(trimethylsilyl)ethoxy)methyl)-1H-pyrazol-3-ol (300 mg, 1.15 mmol), (Cis-Racemate))-4-fluorotetrahydrofuran-3-olin Toluene (6 mL) was added CMBP at RT. The reaction mixture was subjected to heat at 100° C. in a microwave for 1 h. The progress of the reaction was monitored by TLC. After completion of reaction, reaction mixture was evaporated under reduced pressure to give the crude compound. The crude compound was purified by Sepa-Bean using silica gel (100-200 mesh), eluting with 25% ethyl acetate in pet-ether to give Trans-racemate 3-((-4-fluorotetrahydrofuran-3-yl)oxy)-4-nitro-1-((2-(trimethylsilyl)ethoxy)methyl)-1H-pyrazole (220 mg, 54% yield) as a Pale yellow solid. LC-MS m/z [M+H]⁺=348.45

Trans-racemate 3-((-4-fluorotetrahydrofuran-3-yl) oxy)-1-((2-(trimethylsilyl) ethoxy) methyl)-1H-pyrazol-4-amine: To a stirred solution of Trans-racemate 3-((-4-fluorotetrahydrofuran-3-yl)oxy)-4-nitro-1-((2-(trimethylsilyl)ethoxy)methyl)-1H-pyrazole (1.3 g, 3.47 mmol) in Methanol (26 mL), was added 10% Pd/C (40% w/w, 520 mg) at RT and stirred under hydrogen balloon pressure for 4 h. The progress of the reaction was monitored by TLC and LCMS. After completion of reaction, reaction mass was filtered through plug of celite, the filtrate was evaporated under reduced pressure to give Trans-racemate 3-((-4-fluorotetrahydrofuran-3-yl) oxy)-1-((2-(trimethylsilyl) ethoxy) methyl)-1H-pyrazol-4-amine (1.0 g, 84% yield) as a Pale pink syrup. The crude product used as such without further purification. LC-MS m/z [M+H]⁺=318.17

As per the method described in Example 2, coupling to Intermediate 1 and deprotection of SEM followed by chiral SFC provided for Peak 1 2′-((3-(((3R,4R)-4-fluorotetrahydrofuran-3-yl)oxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

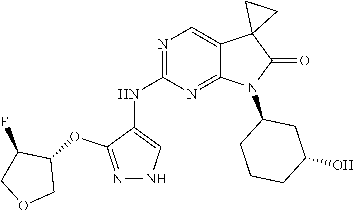 [SMILES: O=C1N([C@@H]2CCC[C@@H](O)C2)c2nc(Nc3c[nH]nc3O[C@@H]3COC[C@H]3F)ncc2C12CC2]

### Example 25

Peak 2 from SFC in example 24 resulted in 2′-((3-(((3S,4S)-4-fluorotetrahydrofuran-3-yl)oxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

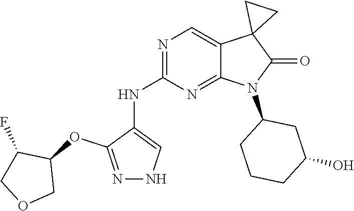 [SMILES: O=C1N([C@@H]2CCC[C@@H](O)C2)c2nc(Nc3c[nH]nc3O[C@H]3COC[C@@H]3F)ncc2C12CC2]

### Example 26

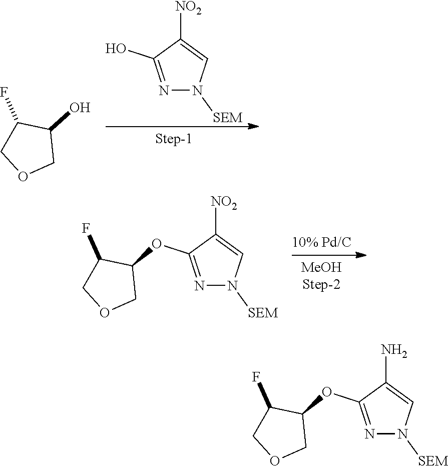 [SMILES: O[C@H]1COC[C@@H]1F ; C[Si](C)(C)CCOCn1cc([N+](=O)[O-])c(O)n1 ; C[Si](C)(C)CCOCn1cc([N+](=O)[O-])c(O[C@H]2COC[C@H]2F)n1 ; C[Si](C)(C)CCOCn1cc(N)c(O[C@H]2COC[C@H]2F)n1]

Synthesis of Cis-Racemate 3-((-4-fluorotetrahydrofuran-3-yl) oxy)-4-nitro-1-((2-(trimethylsilyl) ethoxy) methyl)-1H-pyrazole: To a stirred solution of 4-nitro-1-((2-(trimethylsilyl)ethoxy)methyl)-1H-pyrazol-3-ol (1.0 g, 3.85 mmol), (trans-racemate)-4-fluorotetrahydrofuran-3-ol (0.613 g, 5.78 mmol) and (Tributylphosphoranylidene) acetonitrile (1.86 g, 7.72 mmol) in Toluene (15 mL) at RT. The reaction mixture was subjected to microwave irradiation at 100° C. for 3 h. The progress of the reaction was monitored by TLC. After completion of the reaction, the reaction was evaporated under reduced pressure to give the crude product. The crude compound was purified by Sepa-Bean using silica gel (100-200 mesh), eluting with 30-40% ethyl acetate in pet-ether to give Cis-Racemate 3-((-4-fluorotetrahydrofuran-3-yl)oxy)-1-((2-(trimethylsilyl)ethoxy)methyl)-1H-pyrazol-4-amine (900 mg, 27% yield) as a Pale brown syrup. LC-MS m/z [M+H]⁺=348.19.

Cis-Racemate 3-((-4-fluorotetrahydrofuran-3-yl)oxy)-4-nitro-1-((2-(trimethylsilyl)ethoxy)methyl)-1H-pyrazole: To a stirred solution of Cis-Racemate 3-(-4-fluorotetrahydrofuran-3-yl)oxy)-1-((2-(trimethylsilyl)ethoxy)methyl)-1H-pyrazol-4-amine (0.9 g, 2.59 mmol) in MeOH (9.0 mL) was added 10% Pd/C (40% w/w, 0.36 g) and stirred at RT under hydrogen balloon pressure for 2 h. The progress of the reaction was monitored by TLC. After completion of reaction, reaction mass was filtered through plug of celite, the filtrate was evaporated under reduced pressure to give the crude compound. The crude compound was purified by Sepa-Bean using silica gel (100-200 mesh), eluting with 40-50% ethyl acetate in pet-ether to give Cis-Racemate 3-((-4-fluorotetrahydrofuran-3-yl)oxy)-4-nitro-1-((2-(trimethylsilyl)ethoxy)methyl)-1H-pyrazole (0.3 g) as a Pale brown syrup. LC-MS m/z [M+H]⁺=318.52 As per the method described in Example 2, coupling to Intermediate 1 and deprotection of SEM followed by chiral SFC provided for Peak 1 2′-((3-(((3R,4S)-4-fluorotetrahydrofuran-3-yl)oxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

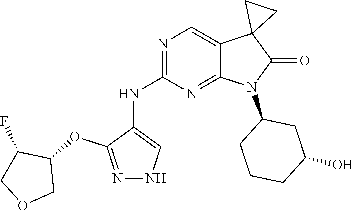 [SMILES: O=C1N([C@@H]2CCC[C@@H](O)C2)c2nc(Nc3c[nH]nc3O[C@@H]3COC[C@@H]3F)ncc2C12CC2]

### Example 27

Peak 2 from SFC in example 26 provided for 2′-((3-(((3S,4R)-4-fluorotetrahydrofuran-3-yl)oxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

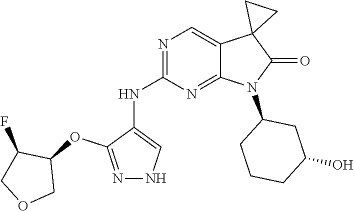 [SMILES: O=C1N([C@@H]2CCC[C@@H](O)C2)c2nc(Nc3c[nH]nc3O[C@H]3COC[C@H]3F)ncc2C12CC2]

### Example 28

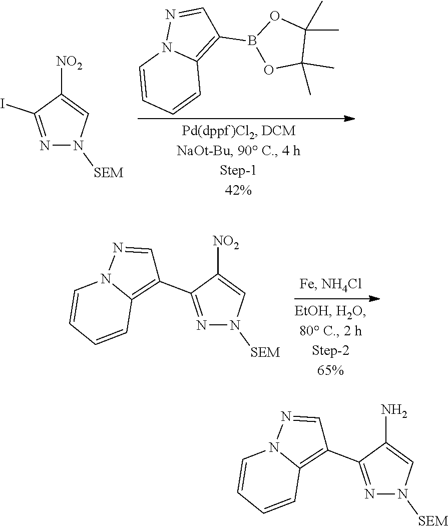 [SMILES: C[Si](C)(C)CCOCn1cc([N+](=O)[O-])c(I)n1 ; CC1(C)OB(c2cnn3ccccc23)OC1(C)C ; C[Si](C)(C)CCOCn1cc([N+](=O)[O-])c(-c2cnn3ccccc23)n1 ; C[Si](C)(C)CCOCn1cc(N)c(-c2cnn3ccccc23)n1]

3-(4-nitro-1-((2-(trimethylsilyl) ethoxy) methyl)-1H-pyrazol-3-yl) pyrazolo[1,5-a] pyridine: To a stirred solution of 3-iodo-4-nitro-1-((2-(trimethylsilyl)ethoxy)methyl)-1H-pyrazole (1.1 g, 2.97 mmol), 3-(4,4,5,5-tetramethyl-1,3,2-dioxaborolan-2-yl)pyrazolo[1,5-a]pyridine (0.729 g, 2.97 mmol) in Toluene:H₂O (20 mL:2 mL) at RT was added NaOt-Bu (0.715 g, 7.42 mmol). The reaction mixture was degassed with argon for 2 min, Pd(dppf)Cl₂·DCM (0.243 g, 0.297 mmol) was added and then stirred at 90° C. for 4 h. The progress of the reaction was monitored by TLC and LCMS. After completion of the reaction, the reaction mass was concentrated under reduced pressure to give crude compound. The crude compound was purified by Sepa-Bean using silica gel (230-400 mesh), eluting with 20-30% ethyl acetate in pet-ether to give 3-(4-nitro-1-((2-(trimethylsilyl)ethoxy)methyl)-1H-pyrazol-3-yl)pyrazolo[1,5-a]pyridine (0.45 g, 42% yield) as a Pale yellow solid. LC-MS m/z [M+H]⁺=360.16

3-(pyrazolo[1,5-a]pyridin-3-yl)-1-((2-(trimethylsilyl)ethoxy)methyl)-1H-pyrazol-4-amine: To a stirring solution of 3-(4-nitro-1-((2-(trimethylsilyl)ethoxy)methyl)-1H-pyrazol-3-yl)pyrazolo[1,5-a]pyridine (350 mg, 0.974 mmol) in Ethanol:H₂O (3.5 mL: 0.7 mL) was added Fe powder (0.272 g, 4.87 mmol), NH₄Cl (0.26 g 0.487 mmol) at RT. The reaction mixture was stirred at 80° C. for 2 h. The progress of the reaction was monitored by TLC and LCMS. After completion of reaction, reaction mass was filtered through plug of celite, the filtrate was concentrated under reduced pressure to give the crude compound. The crude compound was purified by Sepa-Bean using silica gel (100-200 mesh), eluting with 40-45% ethyl acetate in pet-ether pure fractions to give 3-(pyrazolo[1,5-a]pyridin-3-yl)-1-((2-(trimethylsilyl)ethoxy)methyl)-1H-pyrazol-4-amine (0.21 g, 65% yield) as a Pale brown solid. LC-MS m/z [M+H]⁺=330.21

As per the method described in Example 2, coupling to Intermediate 1 and deprotection of SEM provided for 7′-((1R,3R)-3-hydroxycyclohexyl)-2′-((3-(pyrazolo[1,5-a]pyridin-3-yl)-1H-pyrazol-4-yl)amino)spiro[cyclopropane-1,5′-pyrrolo[2,3-d]pyrimidin]-6′(7′H)-one

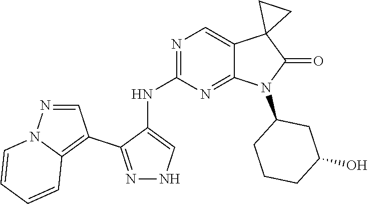 [SMILES: O=C1N([C@@H]2CCC[C@@H](O)C2)c2nc(Nc3c[nH]nc3-c3cnn4ccccc34)ncc2C12CC2]

### Example 29

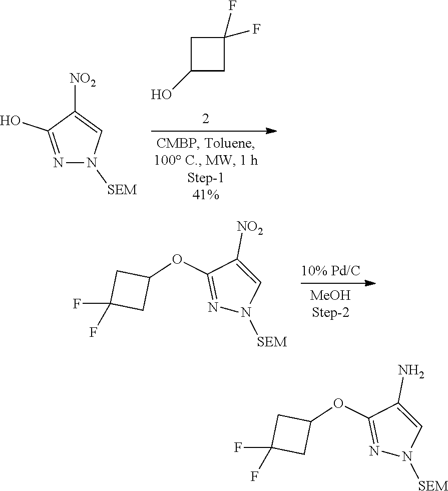 [SMILES: C[Si](C)(C)CCOCn1cc([N+](=O)[O-])c(O)n1 ; OC1CC(F)(F)C1 ; C[Si](C)(C)CCOCn1cc([N+](=O)[O-])c(OC2CC(F)(F)C2)n1 ; C[Si](C)(C)CCOCn1cc(N)c(OC2CC(F)(F)C2)n1]

3-(3,3-difluorocyclobutoxy)-4-nitro-1-((2-(trimethylsilyl)ethoxy)methyl)-1H-pyrazole: To a stirring solution of 4-nitro-1-((2-(trimethylsilyl)ethoxy)methyl)-1H-pyrazol-3-ol (5.0 g, 19.3 mmol), 3,3-difluorocyclobutan-1-ol (2.08 g, 19.3 mmol) in Toluene (50 mL) was added CMBP at RT. The reaction mixture was subjected to heat at 100° C. in a microwave for 1 h. The progress of the reaction was monitored by TLC. After completion of reaction, reaction mixture was evaporated under reduced pressure to give the crude compound. The crude compound was purified by Sepa-Bean using silica gel (100-200 mesh), eluting with 0-10% ethyl acetate in pet-ether to give 3-(3,3-difluorocyclobutoxy)-4-nitro-1-((2-(trimethylsilyl)ethoxy)methyl)-1H-pyrazole (2.8 g, 41% yield) as a Pale brown liquid. ¹H NMR (400 MHz, CDCl₃) δ=8.15 (s, 1H), 5.24 (s, 2H), 4.97-5.00 (m, 1H), 3.60-3.65 (m, 2H), 3.06-3.16 (m, 2H), 2.80-2.93 (m, 2H), 0.91-0.98 (m, 2H), 0.07 (s, 9H).

3-(3,3-difluorocyclobutoxy)-1-((2-(trimethylsilyl)ethoxy)methyl)-1H-pyrazol-4-amine: To a stirring solution of 3-(3,3-difluorocyclobutoxy)-4-nitro-1-((2-(trimethylsilyl)ethoxy)methyl)-1H-pyrazole (2.8 g, 8.022 mmol) in Methanol (28 mL), was added 1% Pd/C (10%, 280 mg) and stirred at RT under hydrogen balloon pressure for 2 h. The progress of the reaction was monitored by TLC. After completion of reaction, reaction mass was filtered through plug of celite, the filtrate was concentrated under reduced pressure to give the 3-(3,3-difluorocyclobutoxy)-1-((2-(trimethylsilyl)ethoxy)methyl)-1H-pyrazol-4-amine (2.5 g) as a Pale brown liquid, proceeded to next step without further purification. ¹H NMR (400 MHz, CDCl₃) δ=7.001 (s, 1H), 5.107 (s, 2H), 3.46-3.51 (m, 2H), 3.05-3.06 (m, 2H), 2.71-2.77 (m, 4H), 0.85-0.90 (m, 2H), 0.001 (s, 9H).

As per the method described in Example 2, coupling to Intermediate 1 and deprotection of SEM provided for 2′-((3-(3,3-difluorocyclobutoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

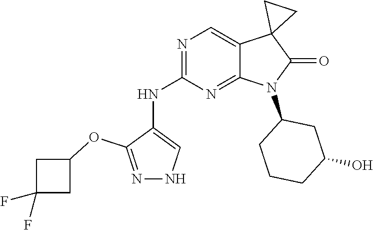 [SMILES: O=C1N([C@@H]2CCC[C@@H](O)C2)c2nc(Nc3c[nH]nc3OC3CC(F)(F)C3)ncc2C12CC2]

### Example 30

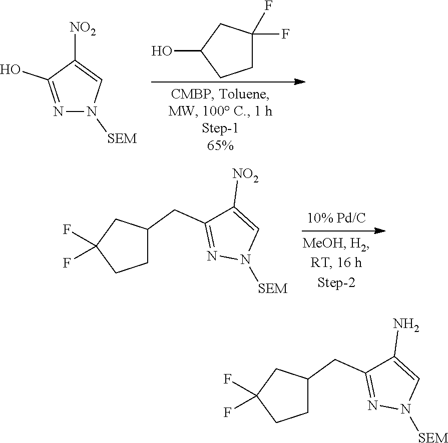 [SMILES: C[Si](C)(C)CCOCn1cc([N+](=O)[O-])c(O)n1 ; OC1CCC(F)(F)C1 ; C[Si](C)(C)CCOCn1cc([N+](=O)[O-])c(CC2CCC(F)(F)C2)n1 ; C[Si](C)(C)CCOCn1cc(N)c(CC2CCC(F)(F)C2)n1]

3-((3,3-difluorocyclopentyl)oxy)-4-nitro-1-((2-(trimethylsilyl)ethoxy)methyl)-1H-pyrazole: To a stirred solution of 4-nitro-1-((2-(trimethylsilyl)ethoxy)methyl)-1H-pyrazol-3-ol (1.000 g, 3.856 mmol), 3,3-difluorocyclopentan-1-ol (470.8 mg, 3.856 mmol) in Toluene (10.00 mL) was added 2-(tributyl-15-phosphaneylidene) acetonitrile (CMBP) (1.861 g, 7.712 mmol). The reaction was subjected to heating at 100° C. for 1 h in micro-wave. The progress of the reaction was monitored by TLC and LCMS. The reaction mass was evaporated under reduced pressure to give crude compound, which was purified by column using silica gel (100-200 mesh), compound was eluted with 30-35% ethyl acetate in pet-ether to give 3-((3,3-difluorocyclopentyl)oxy)-4-nitro-1-((2-(trimethylsilyl)ethoxy)methyl)-1H-pyrazole as a Colour less liquid (920 mg, 65% yield). ¹H NMR (400 MHz, CDCl₃) δ=8.147 (s, 1H), 5.20-5.30 (m, 3H), 3.60-3.68 (m, 2H), 2.58-2.61 (m, 1H), 2.10-2.50 (m, 5H), 0.90-0.95 (m, 2H), 0.002 (s, 9H).

3-((3,3-difluorocyclopentyl) oxy)-1-((2-(trimethylsilyl) ethoxy) methyl)-1H-pyrazol-4-amine: To a stirred solution 3-((3,3-difluorocyclopentyl)oxy)-4-nitro-1-((2-(trimethylsilyl)ethoxy)methyl)-1H-pyrazole (920.0 mg, 1.0, 2.531 mmol) in MeOH (10.00 mL) was added 10% Pd/C (360.0 mg, 40% w/w). The reaction mixture was stirred under H₂ atmosphere (90 psi) at RT for 2 h. The progress of the reaction was monitored by TLC and LCMS. After completion of the reaction, reaction mass was filtered through celite pad. The filtrate was evaporated under reduced pressure to give 3-((3,3-difluorocyclopentyl) oxy)-1-((2-(trimethylsilyl) ethoxy) methyl)-1H-pyrazol-4-amine as a Brown color liquid (820 mg). LC-MS m/z [M+H]⁺=334.13

As per the method described in Example 2, coupling to Intermediate 1 and deprotection of SEM provided for 2′-((3-(3,3-difluorocyclobutoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

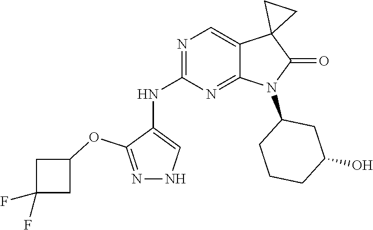 [SMILES: O=C1N([C@@H]2CCC[C@@H](O)C2)c2nc(Nc3c[nH]nc3OC3CC(F)(F)C3)ncc2C12CC2]

### Example 31

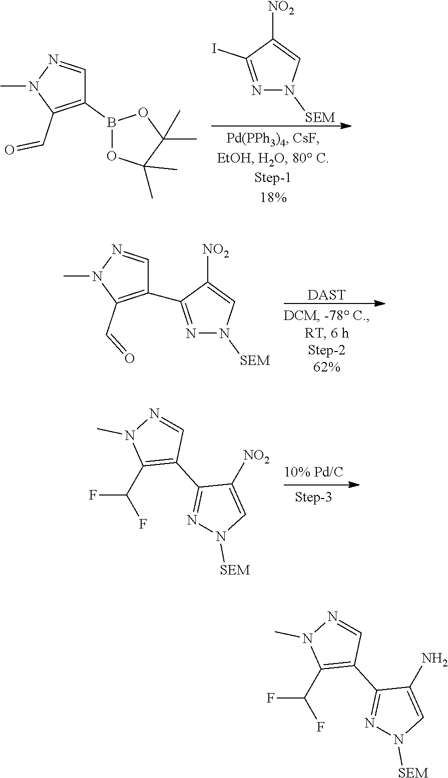

1′-methyl-4-nitro-1-((2-(trimethylsilyl) ethoxy) methyl)-1H,1′H-[3,4′-bipyrazole]-5′-carbaldehyde: To a degassed solution of 1-methyl-4-(4,4,5,5-tetramethyl-1,3,2-dioxaborolan-2-yl)-1H-pyrazole-5-carbaldehyde (1.0 g, 4.23 mmol), 3-iodo-4-nitro-1-((2-(trimethylsilyl)ethoxy)methyl)-1H-pyrazole (1.56 g, 4.23 mmol) in EtOH (4.0 mL), H₂O (1.0 mL) was added CsF (1.28 g, 8.47 mmol) at RT. To reaction mixture Pd(PPh₃)₄ (342 mg, 0.296 mmol) was added and stirred under argon atmosphere at 80° C. for 16 h. The progress of the reaction was monitored by TLC and LCMS. After completion of the reaction, the reaction mass filtered on celite bed and filtrate was evaporated under reduced pressure to give the crude product. The crude compound was purified by Sepa-Bean using silica gel (230-400 mesh), eluting with 10-20% ethyl acetate in pet-ether to give 1′-methyl-4-nitro-1-((2-(trimethylsilyl)ethoxy)methyl)-1H,1′H-[3,4′-bipyrazole]-5′-carbaldehyde (270 mg, 18% yield) as a Pale brown liquid. LC-MS m/z [M+H]⁺=352.54.

5′-(difluoromethyl)-1′-methyl-4-nitro-1-((2-(trimethylsilyl)ethoxy)methyl)-1H,1′H-3,4′-bipyrazole: To a stirred solution of 1′-methyl-4-nitro-1-((2-(trimethylsilyl)ethoxy)methyl)-1H,1′H-[3,4′-bipyrazole]-5′-carbaldehyde (0.42 g, 1.19 mmol) in DCM (8.4 mL) at −78° C. was added DAST (0. ml, 0.47 mmol) drop wise. The reaction mixture was raised to RT and stirred for 6 h. The progress of the reaction was monitored by TLC. After completion of reaction, reaction mass quenched with saturated NaHCO₃ solution, extracted with DCM and evaporated under reduced pressure to give the crude compound. The crude compound was purified by Sepa-Bean using silica gel (230-400 mesh), eluting with 10-20% ethyl acetate in pet-ether to give 5′-(difluoromethyl)-1′-methyl-4-nitro-1-((2-(trimethylsilyl)ethoxy)methyl)-1H,1′H-3,4′-bipyrazole (280 mg, 62% yield) as a Brown liquid. LC-MS m/z [M+H]⁺=374.53.

5′-(difluoromethyl)-1′-methyl-1-((2-(trimethylsilyl)ethoxy)methyl)-1H,1′H-[3,4′-bipyrazol]-4-amine: To a stirring solution of 5′-(difluoromethyl)-1′-methyl-4-nitro-1-((2-(trimethylsilyl)ethoxy)methyl)-1H,1′H-3,4′-bipyrazole (0.28 g, 0.749 mmol) in MeOH (10 mL), was added 10% Pd/C (40% w/w, 112 mg) and stirred at RT under hydrogen balloon pressure for 5 h. The progress of the reaction was monitored by TLC. After completion of reaction, reaction mass was filtered through plug of celite, filtrate was evaporated under reduced pressure to give 5′-(difluoromethyl)-1′-methyl-1-((2-(trimethylsilyl)ethoxy)methyl)-1H,1′H-[3,4′-bipyrazol]-4-amine (0.230 g) as a Pale brown syrup, which was used to next step without further purification. LC-MS m/z [M+H]⁺=344.25

As per the method described in Example 2, coupling to Intermediate 1 and deprotection of SEM provided for 2′-((5′-(difluoromethyl)-1′-methyl-1H,1′H-[3,4′-bipyrazol]-4-yl)amino)-7′-((1R,3R)-3-hydroxycyclohexyl)spiro[cyclopropane-1,5′-pyrrolo[2,3-d]pyrimidin]-6′(7′H)-one

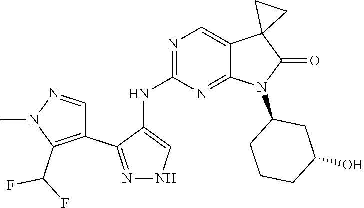 [SMILES: Cn1ncc(-c2n[nH]cc2Nc2ncc3c(n2)N([C@@H]2CCC[C@@H](O)C2)C(=O)C32CC2)c1C(F)F]

### Example 32

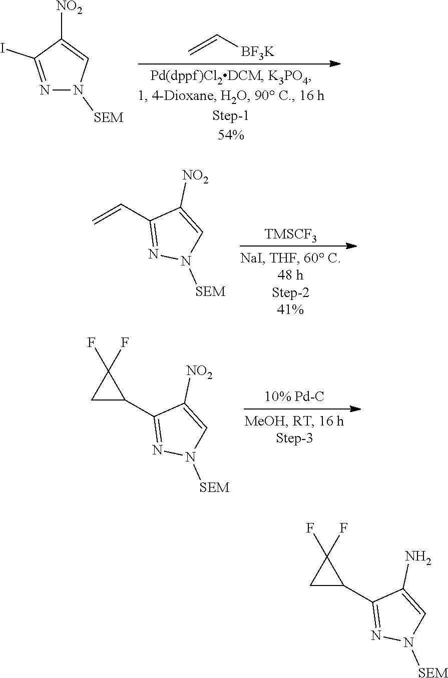

4-nitro-1-((2-(trimethylsilyl)ethoxy)methyl)-3-vinyl-1H-pyrazole: To a stirred solution of 3-iodo-4-nitro-1-((2-(trimethylsilyl)ethoxy)methyl)-1H-pyrazole (5.000 g, 13.54 mmol), Potassium vinyl-trifluoroborate (2.177 g, 16.25 mmol) in 1,4-Dioxane (90.00 mL) was added K₃PO₄ (7.186 g, 33.85 mmol) and H₂O (10 mL) and purged the reaction mass with argon for 5 min. Pd(dppf)Cl₂·DCM (1.106 g, 1.354 mmol) was added and purged the reaction mass with argon for 5 min. The reaction mixture was heated to 90° C. and stirred for 16 h. The progress of the reaction was monitored by LC-MS. After completion of the reaction, reaction mixture was filtered through celite pad and filtrate was evaporated under reduced pressure to give crude product. The crude compound was purified by Sepa-Bean using silica gel (230-400 mesh), eluting with 0-20% ethyl acetate in pet-ether to give 4-nitro-1-((2-(trimethylsilyl)ethoxy)methyl)-3-vinyl-1H-pyrazole as a Pale yellow liquid (2.0 g, 54% yield). ¹H NMR (400 MHz, CDCl₃) δ=8.12 (s, 1H), 7.11-7.18 (m, 1H), 6.24-6.28 (m, 1H), 5.93-5.97 (m, 1H), 5.47 (s, 2H), 3.70-3.74 (m, 2H), 0.91-0.95 (m, 2H), 0.01 (s, 9H).

3-(2,2-difluorocyclopropyl)-4-nitro-1-((2-(trimethylsilyl)ethoxy)methyl)-1H-pyrazole: To a stirred solution of 4-nitro-1-((2-(trimethylsilyl)ethoxy)methyl)-3-vinyl-1H-pyrazole (2.000 g, 7.424 mmol) in THE (40.00 mL) at 0° C. was added NaI (71.27 mg, 2.970 mmol), followed by TMSCF₃ (22.3 mL, 148.5 mmol). The reaction was stirred at 60° C. for 48 h. The progress of the reaction was monitored by TLC and LCMS. After completion of the reaction, the reaction mass was quenched water (30), extracted the compound with ethyl acetate (2×40 mL). The organic layer was dried over Na₂SO₄, evaporated under reduced pressure to give crude compound. The crude compound was purified by Sepa-Bean using silica gel (230-400 mesh), eluting with 15-30% ethyl acetate in pet-ether to give 3-(2,2-difluorocyclopropyl)-4-nitro-1-((2-(trimethylsilyl)ethoxy)methyl)-1H-pyrazole as an Off-white solid (1.1 g, 41% yield). ¹H NMR (400 MHz, CDCl₃) δ=8.12 (s, 1H), 5.42-5.66 (m, 2H), 3.61-3.66 (m, 2H), 2.82-2.88 (m, 1H), 2.17-2.18 (m, 1H), 2.02-2.06 (m, 1H), 0.87-0.95 (m, 2H), 0.008 (s, 9H).

3-(2,2-difluorocyclopropyl)-1-((2-(trimethylsilyl)ethoxy)methyl)-1H-pyrazol-4-amine: To a stirred solution of 3-(2,2-difluorocyclopropyl)-4-nitro-1-((2-(trimethylsilyl)ethoxy)methyl)-1H-pyrazole (1.000 g, 3.131 mmol) in MeOH (30.00 mL) was added 10% Pd—C (400.0 mg, 40% w/w). The reaction mixture was stirred under H₂ (90 psi) at room temperature for 16 h. The progress of the reaction was monitored by TLC and LCMS. After completion of the reaction, reaction mixture was filtered through celite bed, filtrate was evaporated under reduced pressure to give 3-(2,2-difluorocyclopropyl)-1-((2-(trimethylsilyl)ethoxy)methyl)-1H-pyrazol-4-amine as an Off-white solid (900 mg), forwarded to next step with out further purification. LC-MS m/z [M+H]⁺=290.38

As per the method described in Example 2, coupling to Intermediate 1 and deprotection of SEM followed by chiral SFC provided for peak 1 provided for

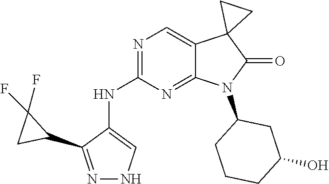

2′-((3-((S)-2,2-difluorocyclopropyl)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

### Example 33

Peak 2 from SFC in example 32 provided for

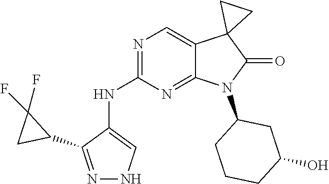

2′-((3-((S)-2,2-difluorocyclopropyl)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

### Example 34

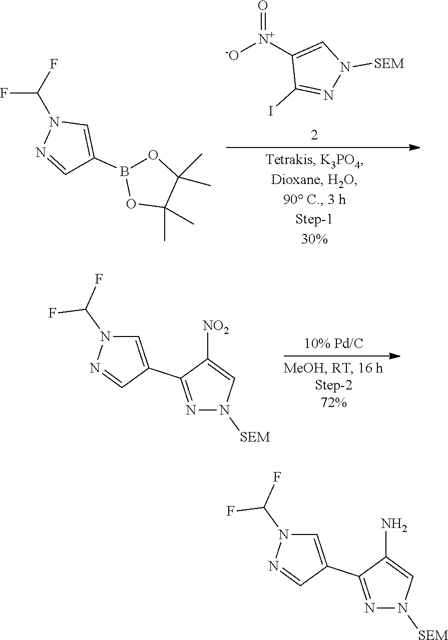

1′-(difluoromethyl)-4-nitro-1-((2-(trimethylsilyl)ethoxy)methyl)-1H,1′H-3,4′-bipyrazole: To a stirred solution of 1-(difluoromethyl)-4-(4,4,5,5-tetramethyl-1,3,2-dioxaborolan-2-yl)-1H-pyrazole (1.0 g, 4.09 mmol) in 1,4-Dioxane (10.00 mL) was added 3-iodo-4-nitro-1-((2-(trimethylsilyl)ethoxy)methyl)-1H-pyrazole (1.96 g, 5.327 mmol), K₃PO₄ (2.17 g, 10.24 mmol), H₂O (2.0 mL) and degassed with the Nitrogen for 5 min. Tetrakis (473.5 mg, 409.8 mmol) was added to the reaction mixture and again degassed with nitrogen for 3 min at RT and then stirred at 90° C. for 3 h. The progress of the reaction was monitored by TLC and LCMS. After completion of reaction, the mixture was filtered through celite bed, then filtrate was concentrated under reduced pressure to get crude compound. The crude was purified by column using 100-200 silica gel, compound was eluted with 50% ethyl acetate in pet-ether to give 1′-(difluoromethyl)-4-nitro-1-((2-(trimethylsilyl)ethoxy)methyl)-1H,1′H-3,4′-bipyrazole as an Off-white solid (440 mg, 30% yield). LC-MS m/z [M+H]+=360.40

1′-(difluoromethyl)-1-((2-(trimethylsilyl)ethoxy)methyl)-1H,1′H-[3,4′-bipyrazol]-4-amine: To a stirred solution of 1′-(difluoromethyl)-4-nitro-1-((2-(trimethylsilyl)ethoxy)methyl)-1H,1′H-3,4′-bipyrazole (440 mg, 1.224 mmol) in MeOH (10 mL) was added 10% Pd/C (130.3 mg). The reaction mixture was stirred under H₂ (70 psi) at room temperature for 16 h. The progress of the reaction was monitored by TLC and LCMS. After completion of the reaction, reaction mixture was filtered through celite bed. The filtrate was concentrated under reduced pressure to get crude compound, which was purified by column using 100-200 silica gel compound was eluted with 60% ethyl acetate in pet-ether to give 1′-(difluoromethyl)-1-((2-(trimethylsilyl)ethoxy)methyl)-1H,1′H-[3,4′-bipyrazol]-4-amine as a brown colour solid (290 mg, 72% yield). LC-MS m/z [M+H]⁺=330.31.

As per the method described in Example 2, coupling to Intermediate 1 and deprotection of SEM provided for

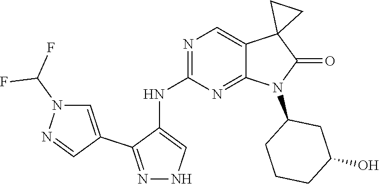 [SMILES: O=C1N([C@@H]2CCC[C@@H](O)C2)c2nc(Nc3c[nH]nc3-c3cnn(C(F)F)c3)ncc2C12CC2]

2′-((1′-(difluoromethyl)-1H,1′H-[3,4′-bipyrazol]-4-yl)amino)-7′-((1R,3R)-3-hydroxycyclohexyl)spiro[cyclopropane-1,5′-pyrrolo[2,3-d]pyrimidin]-6′(7′H)-one

### Example 35

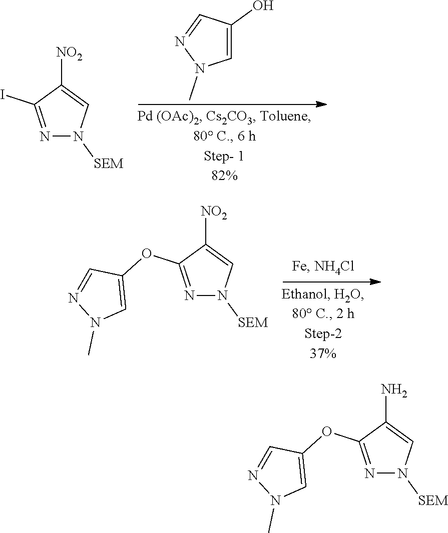

3-((1-methyl-1H-pyrazol-4-yl)oxy)-4-nitro-1-((2-(trimethylsilyl)ethoxy)methyl)-1H-pyrazole: To a stirred solution of 3-iodo-4-nitro-1-((2-(trimethylsilyl)ethoxy)methyl)-1H-pyrazole (1 g, 2.708 mmol) in Toluene (10 mL) was added 1-methyl-1H-pyrazol-4-ol (1.79 g, 2.709 mmol), Cs₂CO₃ (1.324 g, 4.06 mmol) and degassed with the Nitrogen for 5 min. To this 5-(di(adamantan-1-yl) phosphaneyl)-1′,3′,5′-triphenyl-1′H-1,4′-bipyrazole (35.90 mg, 54.17 μmol), Hexakis(acetate) tripalladium(II) (18.24 mg, 27.08 μmol) was added and stirred at 80° C. for 16 h. The progress of the reaction was monitored by TLC and LCMS. The reaction mixture was evaporated under reduced pressure to get crude compound. The crude was purified by column chromatography using 100-200 silica gel, compound was eluted with 40% ethyl acetate in pet-ether to give 3-((1-methyl-1H-pyrazol-4-yl)oxy)-4-nitro-1-((2-(trimethylsilyl)ethoxy)methyl)-1H-pyrazole as an Off-white solid (700 mg, 76% yield). LC-MS m/z [M+H]⁺=340.37 (82%).

3-((1-methyl-1H-pyrazol-4-yl)oxy)-1-((2-(trimethylsilyl)ethoxy)methyl)-1H-pyrazol-4-amine: To a stirred solution of 3-((1-methyl-1H-pyrazol-4-yl)oxy)-4-nitro-1-((2-(trimethylsilyl)ethoxy)methyl)-1H-pyrazole (600 mg, 1.768 mmol) in EtOH (10 mL), H₂O (2.5 mL) at 0° C. was added NH₄Cl (472 mg, 8.838 mmol), Fe (493.6 mg, 8.838 mmol). The reaction mixture was stirred at 80° C. for 2 h. The progress of the reaction was monitored by TLC and LCMS. After completion of the reaction, filtered through a plug of ciliate bed, filtrate was evaporated under reduced pressure to give crude compound, which was purified by column chromatography using 100-200 silica gel, compound eluted with 80% ethyl acetate in pet-ether to give 3-((1-methyl-1H-pyrazol-4-yl)oxy)-1-((2-(trimethylsilyl)ethoxy)methyl)-1H-pyrazol-4-amine as a Brown liquid (200 mg, 37%). LC-MS m/z [M+H]⁺=310.18

As per the method described in Example 2, coupling to Intermediate 1 and deprotection of SEM provided for

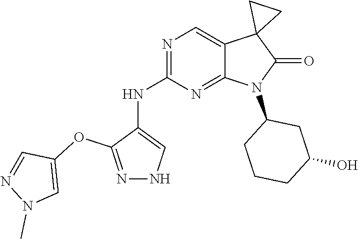 [SMILES: Cn1cc(Oc2n[nH]cc2Nc2ncc3c(n2)N([C@@H]2CCC[C@@H](O)C2)C(=O)C32CC2)cn1]

7′-((1R,3R)-3-hydroxycyclohexyl)-2′-((3-((1-methyl-1H-pyrazol-4-yl)oxy)-1H-pyrazol-4-yl)amino)spiro[cyclopropane-1,5′-pyrrolo[2,3-d]pyrimidin]-6′(7′H)-one

### Example 36

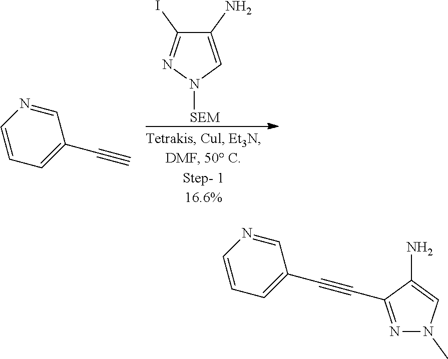

3-(pyridin-3-ylethynyl)-1-((2-(trimethylsilyl)ethoxy)methyl)-1H-pyrazol-4-amine: To a stirred solution of 3-iodo-1-((2-(trimethylsilyl)ethoxy)methyl)-1H-pyrazol-4-amine (600 mg, 1.76 mmol), 3-ethynylpyridine (273 mg, 2.65 mmol) in DMF (5.0 mL) was added Et₃N (6.0 mL), followed by CuI (67.37 mg, 0.36 mmol) and was degassed with the Nitrogen for 5 min. To this Pd(PPh₃)₂Cl₂ (124.1 mg, 6.9 μmol) was added and again degassed with nitrogen for 3 min. The reaction mixture was stirred at 50° C. for 8 h. The progress of the reaction was monitored by TLC and LCMS. After completion of the reaction, the reaction mixture was filtered on celite pad, washed with ethyl acetate. The organic layer was washed with the brine solution and dried over Na₂SO₄ and concentrated under reduced pressure to give the crude product. The crude compound was purified by Sepa-Bean using silica gel (230-400 mesh), eluting with 50-80% ethyl acetate in pet-ether to give 3-(pyridin-3-ylethynyl)-1-((2-(trimethylsilyl)ethoxy)methyl)-1H-pyrazol-4-amine as an Pink gum Liquid (300 mg, 16.6% yield). LC-MS m/z [M+H]⁺=314.46.

As per the method described in Example 2, coupling to Intermediate 1 and deprotection of SEM provided for

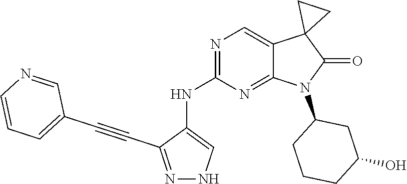 [SMILES: O=C1N([C@@H]2CCC[C@@H](O)C2)c2nc(Nc3c[nH]nc3C#Cc3cccnc3)ncc2C12CC2]

7′-((1R,3R)-3-hydroxycyclohexyl)-2′-((3-(pyridin-3-ylethynyl)-1H-pyrazol-4-yl)amino)spiro[cyclopropane-1,5′-pyrrolo[2,3-d]pyrimidin]-6′(7′H)-one

### Example 37

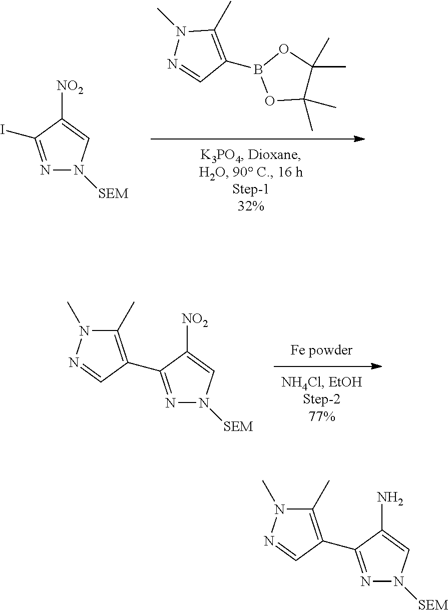

1′,5′-dimethyl-4-nitro-1-((2-(trimethylsilyl)ethoxy)methyl)-1H,1′H-3,4′-bipyrazole: To a stirred solution of 3-iodo-4-nitro-1-((2-(trimethylsilyl)ethoxy)methyl)-1H-pyrazole (500 mg, 1.35 mmol) and 1,5-dimethyl-4-(4,4,5,5-tetramethyl-1,3,2-dioxaborolan-2-yl)-1H-pyrazole (360 mg, 1.62 mmol), Potassium phosphate, tribasic (718.6 mg, 3.385 mmol) in 1,4-Dioxane (9.000 mL) and water (1.000 L) was degassed with the Nitrogen for 5 min. 1,1′Bis(diphenylphosphino)ferrocene] dichloropalladium (II) Complex with Dichloromethane (110.6 mg, 135.4 μmol) was added to the reaction mixture and again degassed with nitrogen for 3 min. The reaction mixture was stirred at 90° C. for 16 h. After completion of the reaction, the reaction mixture was filtered on celite pad, washed with ethyl acetate. The organic layer was washed with the brine solution and dried over Na₂SO₄ and concentrated under reduced pressure to give the crude product. The crude compound was purified by Sepa-Bean using silica gel (230-400 mesh), eluting with 20-30% ethyl acetate in pet-ether to give 1′,5′-dimethyl-4-nitro-1-((2-(trimethylsilyl)ethoxy)methyl)-1H,1′H-3,4′-bipyrazole as an Pale yellow Liquid (250 mg, 32% yield). LC-MS m/z [M+H]⁺=338.45

1′,5′-dimethyl-1-((2-(trimethylsilyl)ethoxy)methyl)-1H,1′H-[3,4′-bipyrazol]-4-amine: To a stirred solution of 1′,5′-dimethyl-4-nitro-1-((2-(trimethylsilyl)ethoxy)methyl)-1H,1′H-3,4′-bipyrazole (250 mg, 0.74 mmol) in Ethanol (2.500 mL) and water (2.500 mL), was added NH₄Cl (198 mg, 3.70 μmol), Fe powder (207 mg, 3.70 mmol) at RT. The mixture was heated to 60° C. and stirred for 4 h. The progress of the reaction was monitored by LC-MS. After completion of reaction, reaction mass was concentrated under reduced pressure to get the crude compound. The crude compound was purified by Sepa-Bean using silica gel (230-400 mesh), eluting with 20-30% ethyl acetate in pet-ether to give 1′,5′-dimethyl-1-((2-(trimethylsilyl)ethoxy)methyl)-1H,1′H-[3,4′-bipyrazol]-4-amine as an Pink colour Liquid (170 mg, 77% yield). LC-MS m/z [M+H]⁺=308.2

As per the method described in Example 2, coupling to Intermediate 1 and deprotection of SEM provided for

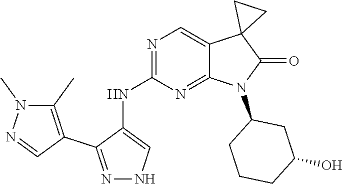 [SMILES: Cc1c(-c2n[nH]cc2Nc2ncc3c(n2)N([C@@H]2CCC[C@@H](O)C2)C(=O)C32CC2)cnn1C]

2′-((1′,5′-dimethyl-1H,1′H-[3,4′-bipyrazol]-4-yl)amino)-7′-((1R,3R)-3-hydroxycyclohexyl)spiro[cyclopropane-1,5′-pyrrolo[2,3-d]pyrimidin]-6′(7′H)-one

### Example 38

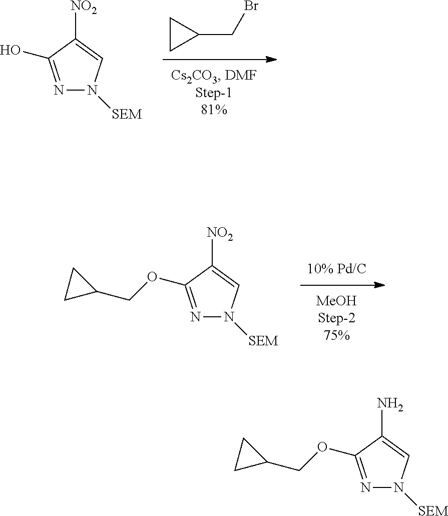 [SMILES: C[Si](C)(C)CCOCn1cc([N+](=O)[O-])c(O)n1 ; BrCC1CC1 ; C[Si](C)(C)CCOCn1cc([N+](=O)[O-])c(OCC2CC2)n1 ; C[Si](C)(C)CCOCn1cc(N)c(OCC2CC2)n1]

3-(cyclopropylmethoxy)-4-nitro-1-((2-(trimethylsilyl)ethoxy)methyl)-1H-pyrazole: To a stirred solution of 4-nitro-1-((2-(trimethylsilyl)ethoxy)methyl)-1H-pyrazol-3-ol (1.0 g, 3.85 mmol) and (bromomethyl)cyclopropane (1.04 g, 7.712 mmol) in DMF (10 mL) at RT was added Cs₂CO₃ (3.141 g, 9.640 mmol) and stirred for 3 h. The progress of the reaction was monitored by TLC and LCMS. After completion of the reaction, quenched with ice water (30 mL) and extracted with ethyl acetate (2×50 mL). The organic layer was dried over Na₂SO₄ and concentrated under reduced pressure to give the crude product. The crude compound was purified by Sepa-Bean using silica gel (230-400 mesh), eluting with 5-10% ethyl acetate in pet-ether to give 3-(cyclopropylmethoxy)-4-nitro-1-((2-(trimethylsilyl)ethoxy)methyl)-1H-pyrazole as a Pale yellow liquid (1.1 g, 81% yield). LC-MS m/z [M+H]⁺=314.42

3-(cyclopropylmethoxy)-1-((2-(trimethylsilyl)ethoxy)methyl)-1H-pyrazol-4-amine: To a stirred solution of 3-(cyclopropylmethoxy)-4-nitro-1-((2-(trimethylsilyl)ethoxy)methyl)-1H-pyrazole (1 g, 3.19 mmol) in Methanol (30 ml) was added 10% Pd/C (300 mg) at room temperature and stirred the reaction mixture under H2 atmosphere in Parr shaker for 5 h. The Progress of the reaction was monitored by TLC. After, completion of the reaction, the reaction mass was filtered through plug of celite, the filtrate was evaporated under reduced pressure to give 3-(cyclopropylmethoxy)-1-((2-(trimethylsilyl)ethoxy)methyl)-1H-pyrazol-4-amine as a Pink liquid (0.9 g, 75% yield). LC-MS m/z [M+H]+=284.46

As per the method described in Example 2, coupling to Intermediate 1 and deprotection of SEM provided for

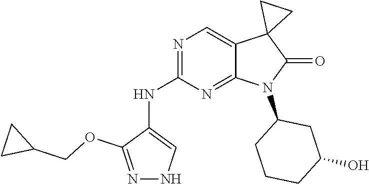 [SMILES: O=C1N([C@@H]2CCC[C@@H](O)C2)c2nc(Nc3c[nH]nc3OCC3CC3)ncc2C12CC2]

2′-((3-(cyclopropylmethoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

### Example 39

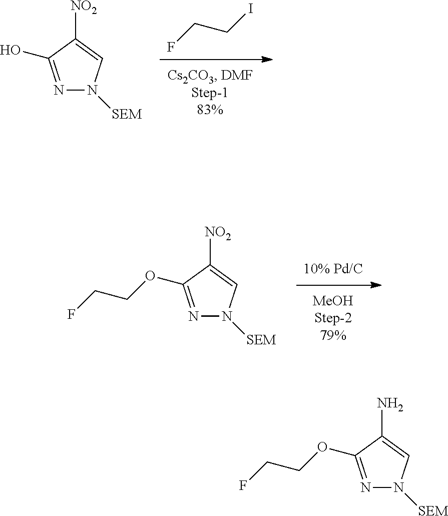

3-(2-fluoroethoxy)-4-nitro-1-((2-(trimethylsilyl)ethoxy)methyl)-1H-pyrazole: To a stirred solution of 4-nitro-1-((2-(trimethylsilyl)ethoxy)methyl)-1H-pyrazol-3-ol (15.0 g, 57.83 mmol) and 1-fluoro-2-iodoethane (20.12 g, 115.68 mmol) in DMF (150 mL), was added Cs₂CO₃ (47.113 g, 144.60 mmol) at RT and stirred for 6 h. The progress of the reaction was monitored by TLC and LCMS. After completion of the reaction, quenched with ice water (100 mL) and extracted with ethyl acetate (3×50 ml). The organic layer was dried over anhydrous Na₂SO₄, filtered and concentrated under reduced pressure to give the crude product. The crude compound was purified by Sepa-Bean using silica gel (230-400 mesh), eluting with 5-10% ethyl acetate in pet-ether to give 3-(2-fluoroethoxy)-4-nitro-1-((2-(trimethylsilyl)ethoxy)methyl)-1H-pyrazole (15 g, 83.61% yield) as an Off-white solid. ¹H NMR (400 MHz, CDCl₃) δ=8.16 (s, 1H), 5.25 (s, 2H), 4.78-4.82 (m, 2H), 4.75-4.77 (m, 1H), 4.61-4.65 (m, 1H), 4.55-4.58 (m, 1H), 3.60-3.65 (m, 2H), 0.92-0.97 (m, 2H), 0.011 (s, 9H).

3-(2-fluoroethoxy)-1-((2-(trimethylsilyl)ethoxy)methyl)-1H-pyrazol-4-amine: To a stirred solution of 3-(2-fluoroethoxy)-4-nitro-1-((2-(trimethylsilyl)ethoxy)methyl)-1H-pyrazole (15 g, 49.119 mmol) in MeOH (150 ml) was added 10% Pd/C (3.0 g, 20% w/w) at RT. The reaction mixture was stirred under H₂ atmosphere in Parr shaker for 5 h. The Progress of the reaction was monitored by TLC. After, completion of the reaction, the reaction mass was filtered through plug of celite, filtrate was concentrated under reduced pressure to give 3-(2-fluoroethoxy)-1-((2-(trimethylsilyl)ethoxy)methyl)-1H-pyrazol-4-amine as a Pink liquid (12.5 g, 79.7% yield). ¹H NMR (400 MHz, CDCl₃) δ=7.03 (s, 1H), 5.13 (s, 2H), 4.68-4.82 (m, 2H), 4.42-4.51 (m, 2H), 3.49-3.53 (m, 2H), 2.77 (br-s, 2H), 0.88-0.92 (m, 2H), 0.00 (s, 9H).

As per the method described in Example 2, coupling to Intermediate 1 and deprotection of SEM provided for

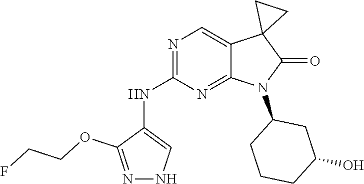 [SMILES: O=C1N([C@@H]2CCC[C@@H](O)C2)c2nc(Nc3c[nH]nc3OCCF)ncc2C12CC2]

2′-((3-(2-fluoroethoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one.

### Example 40

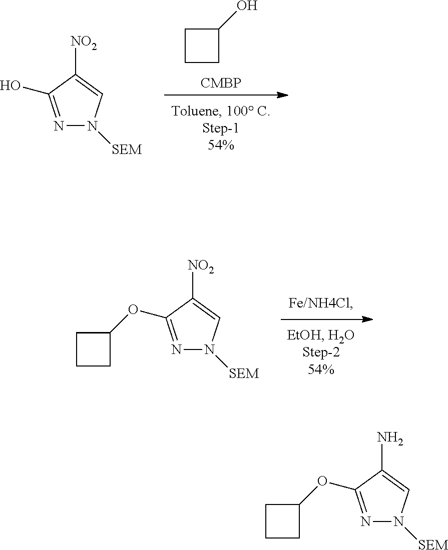

3-cyclobutoxy-4-nitro-1-((2-(trimethylsilyl)ethoxy)methyl)-1H-pyrazole: To a stirred solution of 4-nitro-1-((2-(trimethylsilyl)ethoxy)methyl)-1H-pyrazol-3-ol (600.0 mg, 2.31 mmol) was taken in toluene (6 mL) was added CMBP (1.1 g, 1.23 mL, 4.62 mmol) and cyclobutanol (250.2 mg, 3.47 mmol), Then stirred the reaction mixture for 4 hours at 100° C., Reaction progress was monitored by LC-MS. LC-MS showed desired product formation. Reaction mixture was quenched with water and extracted with ethyl acetate (2×100 ml), combined the organic layers and dried over sodium sulphate filtered and concentrated under reduced vacuum pressure to give crude product. The crude material was absorbed onto a plug of silica gel and purified by sepabean chromatography through a Redi-Sep pre-packed silica gel column (12 g), eluting with a gradient of 0-30% EtOAc in pet ether, collected the pure fractions and concentrated under reduced vacuum pressure to give compound-3 (520.0 mg, 54%, 76% Purity). LC-MS m/z [M+H]+=314.45

3-cyclobutoxy-1-((2-(trimethylsilyl)ethoxy)methyl)-1H-pyrazol-4-amine: To s stirred solution of 3-cyclobutoxy-4-nitro-1-((2-(trimethylsilyl)ethoxy) methyl)-1H-pyrazole (100.0 mg, 0.31 mmol) was taken in Ethanol (1 mL) and water (1 mL) was added ammonium chloride (85.33 mg, 1.59 mmol) and iron (89.09 mg, 1.59 mmol), Then stirred the reaction mixture for 4 hour at 60° C., Reaction progress was monitored by LC-MS & TLC. LC-MS showed desired product formation, TLC showed completion of starting material. Reaction mixture was filtered through celite pad & washed with 10% MeoH in DCM (250 ml), separated the organic layers and concentrated under reduced vacuum pressure to give crude product. this crude product again dried over sodium sulphate filtered and washed with DCM (50 ml) concentrated under reduced vacuum pressure to give crude product 3-cyclobutoxy-1-((2-(trimethylsilyl)ethoxy)methyl)-1H-pyrazol-4-amine (85.00 mg, 54%) as pale brown oil. LC-MS m/z [M+H]⁺=284.33

As per the method described in Example 2, coupling to Intermediate 1 and deprotection of SEM provided for

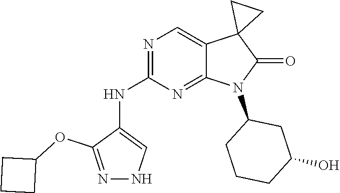 [SMILES: O=C1N([C@@H]2CCC[C@@H](O)C2)c2nc(Nc3c[nH]nc3OC3CCC3)ncc2C12CC2]

2′-((3-cyclobutoxy-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

### Example 41

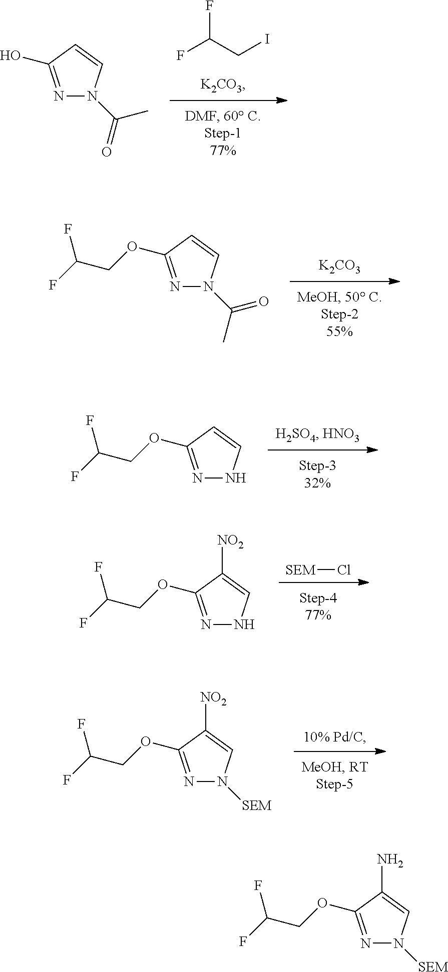

1-(3-hydroxy-1H-pyrazol-1-yl)ethan-1-one: To a degassed solution of 1-(3-hydroxy-1H-pyrazol-1-yl)ethan-1-one (3 g, 23.8 mmol) and 1,1-difluoro-2-iodoethane (6.8 g, 35.71 mmol) in DMF (30 mL), was added Cs₂CO₃ (19.3 g, 59.5 mmol), the reaction mass was closed under argon atmosphere and stirred at 60° C. for 2 h. Progress of the reaction was monitored by TLC and LCMS. After completion of the reaction, the reaction mass was quenched with ice-cold water and extracted with ethyl acetate (2×200 mL). The organic layer was washed with ice-cold water (2×500 mL), dried over Na₂SO₄ and concentrated under reduced pressure to give the crude product. The crude compound was purified by Sepa-Bean using silica gel (230-400 mesh), eluting with 10-30% ethyl acetate in pet-ether to give 1-(3-(2,2-difluoroethoxy)-1H-pyrazol-1-yl)ethan-1-one. (3.5 g, 77% yield) as pale yellow liquid. LC-MS m/z [M+H]+=190.98

3-(2,2-difluoroethoxy)-1H-pyrazole: To a degassed solution of 1-(3-(2,2-difluoroethoxy)-1H-pyrazol-1-yl)ethan-1-one (4.2 g, 22.1 mmol) was taken in MeOH (42 ml), was added K₂CO₃ (4.57, 33.15 mmol), the reaction mass was closed under argon atmosphere and stirred at 50° C. for 1 h. Progress of the reaction was monitored by TLC and LCMS. After completion of the reaction, the reaction mass was filtered through Buchner funnel and washed with 10% MeOH in DCM (200 ml), the filtrates were concentrated under vacuum pressure to give the crude product. 3-(2,2-difluoroethoxy)-1H-pyrazole (3.1 g crude) as pale yellow solid. LC-MS m/z [M+H]+=149.13

3-(2,2-difluoroethoxy)-4-nitro-1H-pyrazole: To a degassed solution of 3-(2,2-difluoroethoxy)-1H-pyrazole (3.5 g, 23.6 mmol) was taken in H₂SO₄ (14 ml 4 vol), was added HNO₃ (3.5 ml, 1 vol), the reaction mass was closed under argon atmosphere and stirred at 50° C. for 3 h. Progress of the reaction was monitored by TLC and LCMS. After completion of the reaction, the reaction mass was quenched with ice-cold water and extracted with ethyl acetate (2×200 mL). The organic layer was washed with ice-cold water (2×500 mL), dried over Na₂SO₄ and concentrated under reduced pressure to give the crude product 3-(2,2-difluoroethoxy)-4-nitro-1H-pyrazole (1.5 g crude, 32% yield) as yellow solid. LC-MS m/z [M+H]+=194.20

3-(2,2-difluoroethoxy)-4-nitro-1-((2-(trimethylsilyl)ethoxy)methyl)-1H-pyrazole: To a stirred solution of 3-(2,2-difluoroethoxy)-4-nitro-1H-pyrazole (1 g, 5.18 mmol) and Cs₂CO₃ (3.36 g, 10.36 mmol) in DMF (10 mL), was added SEM-Cl (1.37 mL, 87.77 mmol) at 0° C. and then stirred at room temperature for 3 h. The progress of the reaction was monitored by TLC and LCMS. After completion of reaction, reaction mass was quenched with ice-cold, water and extracted with ethyl acetate (2×200 mL). The organic layer was washed with ice-cold water (2×500 mL), dried over Na₂SO₄ and concentrated under reduced pressure to give the crude product. The crude compound was purified by Sepa-Bean using silica gel (230-400 mesh), eluting with 10-40% ethyl acetate in pet-ether to give 3-(2,2-difluoroethoxy)-4-nitro-1-((2-(trimethylsilyl)ethoxy)methyl)-1H-pyrazole (1.3 g, 77% yield) as pale yellow colour liquid. LC-MS m/z [M+H]+=324.34

3-(2,2-difluoroethoxy)-1-((2-(trimethylsilyl)ethoxy)methyl)-1H-pyrazol-4-amine: To a parr-shaker containing 3-(2,2-difluoroethoxy)-4-nitro-1-((2-(trimethylsilyl)ethoxy)methyl)-1H-pyrazole (500 mg, 1.547 mmol) in methanol (10 mL), was added Pd/C (10%, 50 mg) and stirred at room temperature at 80 psi hydrogen pressure for 1 h. Progress of the reaction was monitored by TLC. After completion of reaction, reaction mass was filtered through celite pad and washed with 10% MeOH in DCM, the filtrates were concentrated under reduced pressure to give the crude product 3-(2,2-difluoroethoxy)-1-((2-(trimethylsilyl)ethoxy)methyl)-1H-pyrazol-4-amine (350 mg crude) as brown liquid. LC-MS m/z: [M+H]⁺=295.37

As per the method described in Example 2, coupling to Intermediate 1 and deprotection of SEM provided for

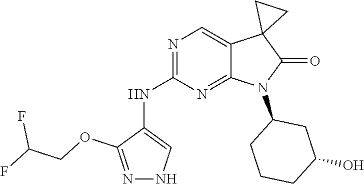 [SMILES: O=C1N([C@@H]2CCC[C@@H](O)C2)c2nc(Nc3c[nH]nc3OCC(F)F)ncc2C12CC2]

2′-((3-(2,2-difluoroethoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

### Example 42

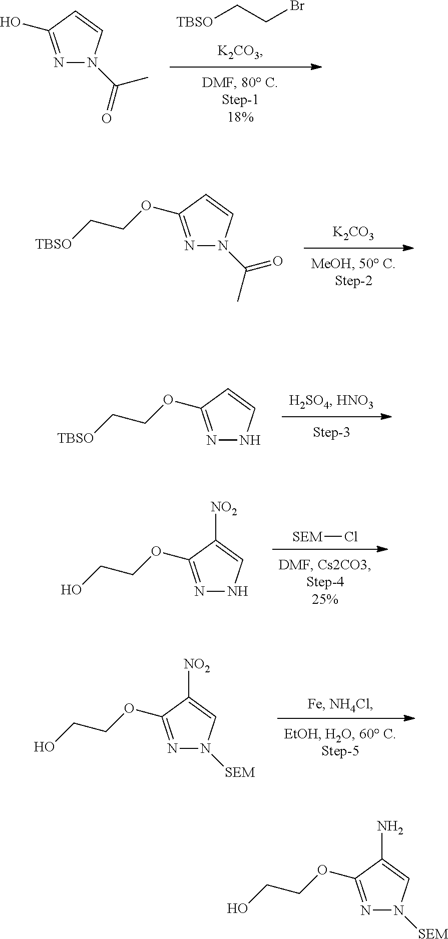

1-(3-(2-((tert-butyldimethylsilyl)oxy)ethoxy)-1H-pyrazol-1-yl)ethan-1-one: To a degassed solution of 1-(3-hydroxy-1H-pyrazol-1-yl)ethan-1-one (6 g, 47.6 mmol) and (2-bromoethoxy)(tert-butyl)dimethylsilane (12.46 g, 52.3 mmol) in DMF (30 mL), was added K₂CO₃ (9.8 g, 71.4 mmol), the reaction mass was closed under argon atmosphere and stirred at 80° C. for 4 h. Progress of the reaction was monitored by TLC and LCMS. After completion of the reaction, the reaction mass was quenched with ice-cold water and extracted with ethyl acetate (2×200 mL). The organic layer was washed with ice-cold water (2×200 mL), dried over Na₂SO₄ and concentrated under reduced pressure to give the crude product. The crude compound was purified by Sepa-Bean using silica gel (230-400 mesh), eluting with 10-30% ethyl acetate in pet-ether to give 1-(3-(2-((tert-butyldimethylsilyl)oxy)ethoxy)-1H-pyrazol-1-yl)ethan-1-one. (2.5 g, 18% yield) as pale yellow liquid. LC-MS m/z [M+H]+=285.27

3-(2-((tert-butyldimethylsilyl)oxy)ethoxy)-1H-pyrazole: To a degassed solution of 1-(3-(2-((tert-butyldimethylsilyl)oxy)ethoxy)-1H-pyrazol-1-yl)ethan-1-one (2.2 g, 7.746 mmol) was taken in MeOH (22 ml), was added K₂CO₃ (11.6 g, 11.61 mmol), the reaction mass was closed under argon atmosphere and stirred at 50° C. for 1 h. Progress of the reaction was monitored by TLC and LCMS. After completion of the reaction, the reaction mass was filtered through Buchner funnel and washed with 10% MeOH in DCM (200 ml) the filtrates were concentrated under vacuum pressure to give the crude product 3-(2-((tert-butyldimethylsilyl)oxy)ethoxy)-1H-pyrazole (1.55 g crude) as pale yellow solid. LC-MS m/z [M+H]+=243.42

2-((4-nitro-1H-pyrazol-3-yl)oxy)ethan-1-ol: To a degassed solution of 3-(2-((tert-butyldimethylsilyl)oxy)ethoxy)-1H-pyrazole (600 mg, 2.479 mmol) was taken in H₂SO₄ (2.4 ml 4 vol), was added HNO₃ (0.6 ml, 1 vol), the reaction mass was closed under argon atmosphere and stirred at 50° C. for 3 h. Progress of the reaction was monitored by TLC and LCMS. After completion of the reaction, the reaction mass was quenched with ice-cold water and extracted with ethyl acetate (2×50 mL). The organic layer was washed with ice-cold water (2×100 mL), dried over Na₂SO₄ and concentrated under reduced pressure to give the crude product 2-((4-nitro-1H-pyrazol-3-yl)oxy)ethan-1-ol (400 mg crude) as pale yellow liquid. LC-MS m/z [M+H]+=174.03

2-((4-nitro-1-((2-(trimethylsilyl)ethoxy)methyl)-1H-pyrazol-3-yl)oxy)ethan-1-ol: To a stirred solution of 2-((4-nitro-1H-pyrazol-3-yl)oxy)ethan-1-ol (400 mg, 2.312 mmol) and Cs₂CO₃ (1.502 g, 4.624 mmol) in DMF (10 mL), was added SEM-Cl (0.61 mL, 3.468 mmol) at 0° C. and then stirred at room temperature for 16 h. The progress of the reaction was monitored by TLC and LCMS. After completion of reaction, reaction mass was quenched with ice-cold water and extracted with ethyl acetate (2×100 mL). The organic layer was washed with ice-cold water (2×200 mL), dried over Na₂SO₄ and concentrated under reduced pressure to give the crude product. The crude compound was purified by Sepa-Bean using silica gel (230-400 mesh), eluting with 10-40% ethyl acetate in pet-ether to give 2-((4-nitro-1-((2-(trimethylsilyl)ethoxy)methyl)-1H-pyrazol-3-yl)oxy)ethan-1-ol (180 mg, 25% yield) as Pale yellow colour liquid. LC-MS m/z [M+H]+=304.12

2-((4-amino-1-((2-(trimethylsilyl)ethoxy)methyl)-1H-pyrazol-3-yl)oxy)ethan-1-ol: To a stirred solution of Compound-7 (180 mg 0.594 mmol) in ethanol (10V) and water (10V) was added NH₄Cl (317.1 mg, 5.94 mmol) followed by Fe (331.4 g, 5.94 mmol) and the reaction mixture was stirred at 60° C. for 1 h. The progress of the reaction was monitored by TLC. The reaction mass was filtered through plug of celite and washed with 10% MeOH in DCM; the filtrate was washed with water (50 mL) and extracted with 10% MeOH in DCM concentrated under reduced pressure to give Compound-8 as brown gummy liquid (169 mg, crude). LC-MS m/z [M+H]+=274.19

As per the method described in Example 2, coupling to Intermediate 1 and deprotection of SEM provided for

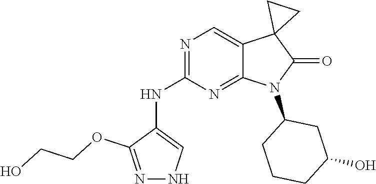 [SMILES: O=C1N([C@@H]2CCC[C@@H](O)C2)c2nc(Nc3c[nH]nc3OCCO)ncc2C12CC2]

7′-((1R,3R)-3-hydroxycyclohexyl)-2′-((3-(2-hydroxyethoxy)-1H-pyrazol-4-yl)amino)spiro[cyclopropane-1,5′-pyrrolo[2,3-d]pyrimidin]-6′(7′H)-one

### Example 43

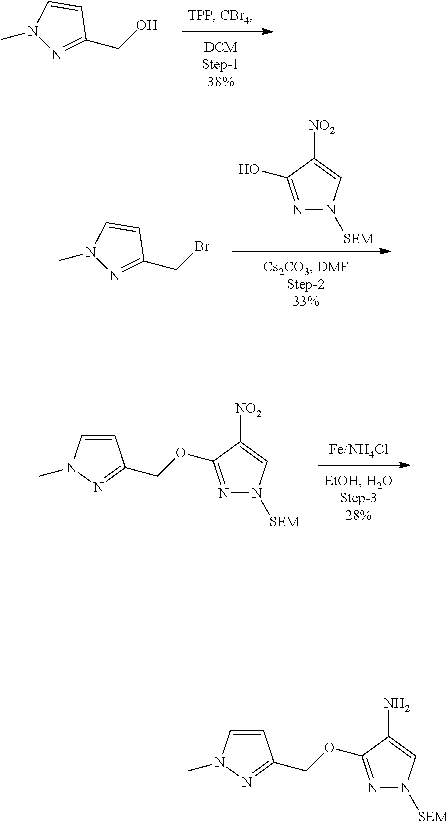

4-nitro-1-((2-(trimethylsilyl)ethoxy)methyl)-1H-pyrazol-3-ol: To stirred solution of (1-methylpyrazol-3-yl) methanol (1.000 g, 1 Eq, 8.918 mmol) in DCM (100.0 mL) was added Triphenylphosphine (3.977 g, 3.359 mL, 15.16 mmol) and carbontetrabromide (5.324 g, 1.68 mL, 16.05 mmol), Then stirred the reaction mixture for 3 hour at RT, Reaction progress was monitored by LC-MS and TLC. LC-MS showed desired product formation. Reaction mixture was quenched with water (25 ml) and extracted with ethyl acetate (2×50 ml), combined the organic layers and dried over sodium sulphate filtered and concentrated under reduced vacuum pressure to give crude product. The crude material was absorbed onto a plug of silica gel and purified by sepabean chromatography through a Redi-Sep pre-packed silica gel column (24 g), eluting with a gradient of 15-30% EtOAc in petether, collected the pure fractions and concentrated under reduced vacuum pressure to give 4-nitro-1-((2-(trimethylsilyl)ethoxy)methyl)-1H-pyrazol-3-ol (900.0 mg, 38.91%) as a yellow oil. LC-MS m/z [M+H]⁺=176.91

3-((1-methyl-1H-pyrazol-3-yl)methoxy)-4-nitro-1-((2-(trimethylsilyl)ethoxy)methyl)-1H-pyrazole: To a stirred solution of 4-nitro-1-((2-(trimethylsilyl) ethoxy) methyl)-1H-pyrazol-3-ol (600.0 mg, 2.314 mmol) in DMF (3.000 mL) was added Cesium carbonate (1.885 g, 462.8 μL, 5.784 mmol) and 3-(bromomethyl)-1-methyl-1H-pyrazole (688.4 mg, 3.933 mmol) Then stirred the reaction mixture for 3 hour at RT. Reaction progress was monitored by LC-MS. LC-MS showed desired product formation. Reaction mixture was quenched with cold water (100 ml) and extracted with ethyl acetate (2×100 ml), combined the organic layers and dried over sodium sulphate filtered and concentrated under reduced vacuum pressure to give 3-((1-methyl-1H-pyrazol-3-yl) methoxy)-4-nitro-1-((2-(trimethylsilyl)ethoxy)methyl)-1H-pyrazole (550.0 mg, 33.64%) as brown gummy solid. LC-MS m/z [M+H]⁺=353.47.

3-((1-methyl-1H-pyrazol-3-yl)methoxy)-1-((2-(trimethylsilyl)ethoxy)methyl)-1H-pyrazol-4-amine: To stirred solution of 3-((1-methyl-1H-pyrazol-3-yl)methoxy)-4-nitro-1-((2-(trimethylsilyl)ethoxy) methyl)-1H-pyrazole (500.0 mg, 1.415 mmol) in Ethanol (5.000 mL) and water (5.000 mL) and was added Ammonium chloride (378.3 mg, 262.9 μL 7.073 mmol) and Iron (395.0 mg, 50.3 μL, 7.073 mmol) Then stirred the reaction mixture for 3 hour at 50° C. Reaction progress was monitored by LC-MS and TLC. LC-MS showed desired product formation. Reaction mixture was filtered through celite pad washed with 10% MEOH in DCM and filtrate is collected and concentrated under reduced vacuum pressure to give crude product. The crude material was washed with DCM to remove ammonium chloride which remains as solid and desired compound is soluble in DCM is collected and concentrated under reduced vacuum pressure to give 3-((1-methyl-1H-pyrazol-3-yl)methoxy)-1-((2-(trimethylsilyl)ethoxy)methyl)-1H-pyrazol-4-amine (300.0 mg, 28.38%) as brown gummy solid. LC-MS m/z [M+H]⁺=323.47

As per the method described in Example 2, coupling to Intermediate 1 and deprotection of SEM provided for

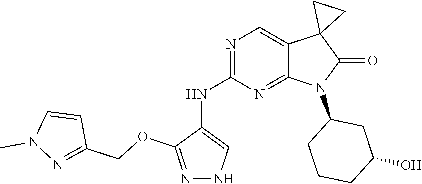 [SMILES: Cn1ccc(COc2n[nH]cc2Nc2ncc3c(n2)N([C@@H]2CCC[C@@H](O)C2)C(=O)C32CC2)n1]

7′-((1R,3R)-3-hydroxycyclohexyl)-2′-((3-((1-methyl-1H-pyrazol-3-yl)methoxy)-1H-pyrazol-4-yl)amino)spiro[cyclopropane-1,5′-pyrrolo[2,3-d]pyrimidin]-6′(7′H)-one

### Example 44

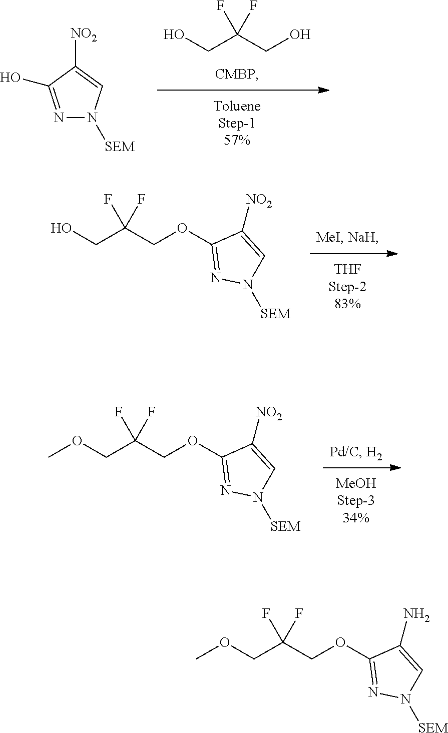 [SMILES: C[Si](C)(C)CCOCn1cc([N+](=O)[O-])c(O)n1 ; OCC(F)(F)CO ; C[Si](C)(C)CCOCn1cc([N+](=O)[O-])c(OCC(F)(F)CO)n1 ; COCC(F)(F)COc1nn(COCC[Si](C)(C)C)cc1[N+](=O)[O-] ; "Pd/C, H2" ; COCC(F)(F)COc1nn(COCC[Si](C)(C)C)cc1N]

2,2-difluoro-3-((4-nitro-1-((2-(trimethylsilyl)ethoxy)methyl)-1H-pyrazol-3-yl)oxy)propan-1-ol: To a stirred solution of 4-nitro-1-((2-(trimethylsilyl)ethoxy)methyl)-1H-pyrazol-3-ol (300.0 mg, 1.157 mmol) in Toluene (9.000 mL), CMBP (558.4 mg, 612 μL, 2.314 mmol) and 2,2-Difluoropropane-1,3-diol (194.5 mg, 1.735 mmol) is added, then stirred the reaction mixture for 16 hour at 100° C. Reaction progress was monitored by TLC and LC-MS. TLC and LC-MS shows desired product formation. Reaction mixture was quenched with water (50 ml) and extracted with ethyl acetate (2×100 ml), combined the organic layers and dried over sodium sulphate filtered and concentrated under reduced vacuum pressure to give crude product. The crude material was absorbed onto a plug of silica gel and purified by sepabean chromatography through a Redi-Sep pre-packed silica gel column (12 g), eluting with a gradient of 21.2% EtOAc in pet ether, collected the pure fractions and concentrated under reduced vacuum pressure to give 2,2-difluoro-3-((4-nitro-1-((2-(trimethylsilyl)ethoxy)methyl)-1H-pyrazol-3-yl)oxy)propan-1-ol (300.0 mg, 57%) as light brown liquid. LC-MS m/z [M+H]+=354.38

3-(2,2-difluoro-3-methoxypropoxy)-4-nitro-1-((2-(trimethylsilyl)ethoxy)methyl)-1H-pyrazole: To stirred solution of 2,2-difluoro-3-((4-nitro-1-((2-(trimethylsilyl)ethoxy)methyl)-1H-pyrazol-3-yl)oxy)propan-1-ol (250.00 mg, 707.41 μmol) in THE (3.250 mL) and Sodium hydride (43.91 mg, 1.0611 mmol) is added at 0° C. and after 15 minutes Methyl iodide (200.82 mg, 1.4148 mmol) is added, Then stirred the reaction mixture for 1 hour at RT, Reaction progress was monitored by TLC and LC-MS. LC-MS showed desired product formation. Reaction mixture was quenched with ice water (100 ml) and extracted with ethyl acetate (2×100 ml), combined the organic layers and dried over sodium sulphate filtered and concentrated under reduced vacuum pressure to give crude product. The crude material was absorbed onto a plug of silica gel and purified by sepabean chromatography through a Redi-Sep pre-packed silica gel column (12 g), eluting with a gradient of 20-22% EtOAc in pet ether, collected the pure fractions and concentrated under reduced vacuum pressure to give 3-(2,2-difluoro-3-methoxypropoxy)-4-nitro-1-((2-(trimethylsilyl)ethoxy)methyl)-1H-pyrazole (240.0 mg, 83%) as light brown gummy liquid. LC-MS m/z [M+H]⁺=368.42

3-(2,2-difluoro-3-methoxypropoxy)-1-((2-(trimethylsilyl)ethoxy)methyl)-1H-pyrazol-4-amine: To stirred solution of 3-(2,2-difluoro-3-methoxypropoxy)-4-nitro-1-((2-(trimethylsilyl)ethoxy)methyl)-1H-pyrazole (240.0 mg, 653.2 μmol) was taken in Methanol (2.500 mL) and Pd/c (69.51 mg, 65.32 μmol) was added and H₂ gas is passed through bladder at 5 psi pressure, then stirred the reaction mixture for 4 hours at RT Reaction progress was monitored by LC-MS and TLC. LC-MS showed desired product formation. Reaction mixture was filtered through celite pad and washed with 10% MEOH in DCM and filtrate is collected and concentrated under reduced vacuum pressure to give 3-(2,2-difluoro-3-methoxypropoxy)-1-((2-(trimethylsilyl)ethoxy)methyl)-1H-pyrazol-4-amine (150.0 mg, 34%). as brown gummy solid. LC-MS m/z [M+H]+=338.42

As per the method described in Example 2, coupling to Intermediate 1 and deprotection of SEM provided for

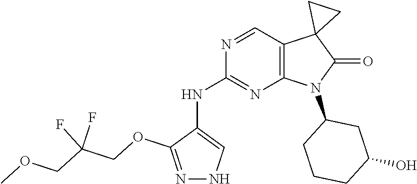 [SMILES: COCC(F)(F)COc1n[nH]cc1Nc1ncc2c(n1)N([C@@H]1CCC[C@@H](O)C1)C(=O)C21CC1]

2′-((3-(2,2-difluoro-3-methoxypropoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

### Example 45

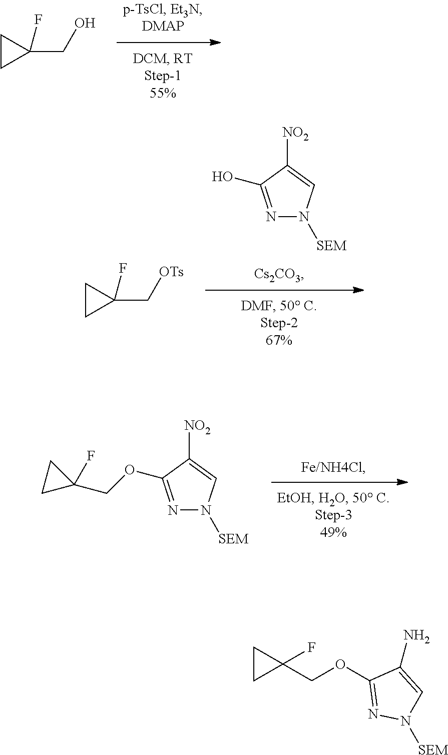 [SMILES: OCC1(F)CC1 ; C[Si](C)(C)CCOCn1cc([N+](=O)[O-])c(O)n1 ; Cc1ccc(S(=O)(=O)OCC2(F)CC2)cc1 ; C[Si](C)(C)CCOCn1cc([N+](=O)[O-])c(OCC2(F)CC2)n1 ; "Fe/NH4Cl," ; C[Si](C)(C)CCOCn1cc(N)c(OCC2(F)CC2)n1]

(1-fluorocyclopropyl)methyl 4-methylbenzenesulfonate: To a stirred solution of (1-fluorocyclopropyl)methanol (1.000 g, 11.10 mmol) in DCM (10.000 mL), Triethylamine (7.73 mL, 55.49 mmol) was added and DMAP (271.2 mg, 2.220 mmol), p-Toluenesulfonyl chloride (3.174 g, 16.65 mmol) is added at 0° C., then stirred the reaction mixture for 3 hours at RT, Reaction progress was monitored by LC-MS and TLC. TLC showed completion of SM-1. (No LC-MS ionization.) Reaction mixture was quenched with NaHCO₃ solution (100 ml) and extracted with ethyl acetate (2×100 ml), combined the organic layers and dried over sodium sulphate filtered and concentrated under reduced vacuum pressure to give crude product. The crude material was absorbed onto a plug of silica gel and purified by sepabean chromatography through a Redi-Sep pre-packed silica gel column (12 g), eluting with a gradient of 0-30% EtOAc in pet ether, collected the pure fractions and concentrated under reduced vacuum pressure to give (1-fluorocyclopropyl)methyl 4-methylbenzenesulfonate (1.500 g, 55.33%) as white solid. LC-MS m/z [M+H]+=245.38

3-((1-fluorocyclopropyl)methoxy)-4-nitro-1-((2-(trimethylsilyl)ethoxy)methyl)-1H-pyrazole: To a stirred solution of 4-nitro-1-((2-(trimethylsilyl)ethoxy)methyl)-1H-pyrazol-3-ol (1.000 g, 3.856 mmol) in DMF (5.500 mL) was added Cesium carbonate (3.141 g, 9.640 mmol) and (1-fluorocyclopropyl)methyl 4-methylbenzenesulfonate (1.413 g, 5.784 mmol), then stirred the reaction mixture for 2 hour at 50° C., Reaction progress was monitored by LC-MS and TLC. LC-MS showed desired product formation. Reaction mixture was quenched with cold water (100 ml) and extracted with ethyl acetate (2×100 ml), combined the organic layers and dried over sodium sulphate filtered and concentrated under reduced vacuum pressure to give crude product. The crude material was absorbed onto a plug of silica gel and purified by sepabean chromatography through a Redi-Sep pre-packed silica gel column (24 g), eluting with a gradient of 0-30% EtOAc in pet ether, collected the pure fractions and concentrated under reduced vacuum pressure to give 3-((1-fluorocyclopropyl)methoxy)-4-nitro-1-((2-(trimethylsilyl)ethoxy)methyl)-1H-pyrazole (1.000 g, 67%) as colourless gummy liquid. LC-MS m/z [M+H]⁺=332.2

3-((1-fluorocyclopropyl)methoxy)-1-((2-(trimethylsilyl)ethoxy)methyl)-1H-pyrazol-4-amine: To a stirred solution of 3-((1-fluorocyclopropyl)methoxy)-4-nitro-1-((2-(trimethylsilyl)ethoxy)methyl)-1H-pyrazole (700.0 mg, 2.112 mmol) in Ethanol (7.000 mL) and Water (7.000 mL) and Ammonium chloride (564.9 mg, 10.56 mmol) and Iron (589.8 mg, 10.56 mmol) Then stirred the reaction mixture for 3 hour at 50° C. Reaction progress was monitored by LC-MS and TLC. LC-MS and TLC showed desired product formation. Reaction mixture was filtered through celite pad and washed with 10% MEOH in DCM and filtrate is collected and concentrated under reduced vacuum pressure to give crude product. The crude material was washed with DCM to remove ammonium chloride which remains as solid and desired compound is soluble in DCM is collected and concentrated under reduced vacuum pressure to give 3-((1-fluorocyclopropyl)methoxy)-1-((2-(trimethylsilyl)ethoxy)methyl)-1H-pyrazol-4-amine (600.0 mg, 49%) as brown gummy solid. LC-MS m/z [M+H]⁺=302.2

As per the method described in Example 2, coupling to Intermediate 1 and deprotection of SEM provided for

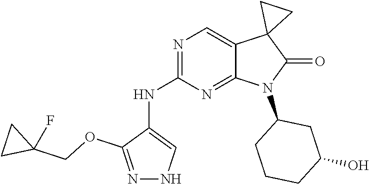 [SMILES: O=C1N([C@@H]2CCC[C@@H](O)C2)c2nc(Nc3c[nH]nc3OCC3(F)CC3)ncc2C12CC2]

2′-((3-((1-fluorocyclopropyl)methoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

### Example 46

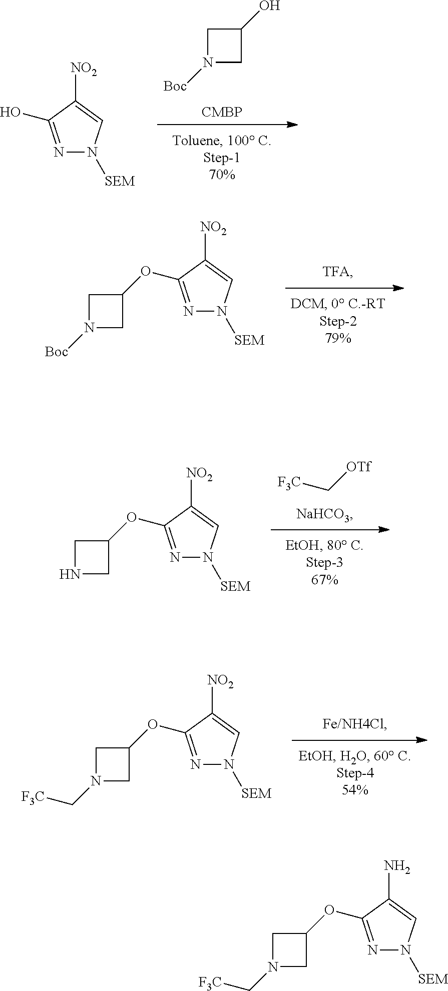

tert-butyl 3-((4-nitro-1-((2-(trimethylsilyl)ethoxy)methyl)-1H-pyrazol-3-yl)oxy)azetidine-1-carboxylate: To a stirred solution of 4-nitro-1-((2-(trimethylsilyl)ethoxy)methyl)-1H-pyrazol-3-ol (500.0 mg, 1.928 mmol) in Toluene (5.000 mL), CMBP (930.7 mg, 1.02 mL, 3.856 mmol) and tert-butyl 3-hydroxyazetidine-1-carboxylate (500.9 mg, 2.892 mmol) is added, Then stirred the reaction mixture for 4 hour at 100° C. Reaction progress was monitored by, TLC and LC-MS. TLC and LC-MS shows no proper ionization but TLC Shows desired product formation. Reaction mixture was quenched with NaHCO₃ (100 ml) and extracted with ethyl acetate (2×100 ml), combined the organic layers and dried over sodium sulphate filtered and concentrated under reduced vacuum pressure to give crude product. The crude material was absorbed onto a plug of silica gel and purified by sepabean chromatography through a Redi-Sep pre-packed silica gel column (12 g), eluting with a gradient of 0-30% EtOAc in pet ether, collected the pure fractions and concentrated under reduced vacuum pressure to give tert-butyl 3-((4-nitro-1-((2-(trimethylsilyl)ethoxy)methyl)-1H-pyrazol-3-yl)oxy)azetidine-1-carboxylate (700.0 mg, 70%) as light brown liquid. LC-MS m/z [M+H]⁺=414.53

3-(azetidin-3-yloxy)-4-nitro-1-((2-(trimethylsilyl)ethoxy)methyl)-1H-pyrazole: To a stirred solution of tert-butyl 3-((4-nitro-1-((2-(trimethylsilyl)ethoxy)methyl)-1H-pyrazol-3-yl)oxy)azetidine-1-carboxylate (700.00 mg, 1.68 mmol) in DCM (7.000 mL) was added TFA (1.400 mL) and then stirred the reaction mixture for 1 hour at RT, Reaction progress was monitored by LC-MS. LC-MS showed desired product formation. Reaction mixture was quenched with Cold sat. NAHCO3 solution (100 ml) and extracted with 10% MeOH IN DCM (2×100 ml), combined the organic layers and dried over sodium sulphate filtered and concentrated under reduced vacuum pressure to give 3-(azetidin-3-yloxy)-4-nitro-1-((2-(trimethylsilyl)ethoxy)methyl)-1H-pyrazole (440.0 mg, 79%) as colorless gummy solid. LC-MS m/z [M+H]+=314.53

4-nitro-3-((1-(2,2,2-trifluoroethyl)azetidin-3-yl)oxy)-1-((2-(trimethylsilyl)ethoxy)methyl)-1H-pyrazole: To a stirred solution of 3-(azetidin-3-yloxy)-4-nitro-1-((2-(trimethylsilyl)ethoxy)methyl)-1H-pyrazole (440.00 mg, 1 Eq, 1.3994 mmol) in EtOH (8.800 mL) was Sodium bicarbonate (470.3 mg, 218 μL, 4 Eq, 5.5976 mmol) and 2,2,2-trifluoroethyl trifluoromethanesulfonate (649.60 mg, 2 Eq, 2.7988 mmol) was added at 00C, Then stirred the reaction mixture for 16 hour at 80° C., Reaction progress was monitored by TLC AND LC-MS. LC-MS showed desired product formation. Reaction mixture was quenched with water (25 ml) and extracted with DCM (1×100 ml), combined the organic layers and dried over sodium sulphate filtered and concentrated under reduced vacuum pressure to give 4-nitro-3-((1-(2,2,2-trifluoroethyl)azetidin-3-yl)oxy)-1-((2-(trimethylsilyl)ethoxy)methyl)-1H-pyrazole (400.0 mg, 67%) as brown gummy solid. LC-MS m/z [M+H]⁺=396.44

3-((1-(2,2,2-trifluoroethyl)azetidin-3-yl)oxy)-1-((2-(trimethylsilyl)ethoxy)methyl)-1H-pyrazol-4-amine: To stirred solution of 4-nitro-3-(oxetan-3-ylmethoxy)-1-((2-(trimethylsilyl)ethoxy)methyl)-1H-pyrazole (450.0 mg, 1.366 mmol) as taken in Ethanol (4.500 mL) and Water (4.500 mL) and was added Ammonium chloride (365.3 mg, 6.830 mmol) and Iron (381.5 mg, 6.830 mmol) Then stirred the reaction mixture for 3 hour at 50° C. Reaction progress was monitored by LC-MS and TLC. LC-MS showed desired product formation. Reaction mixture was filtered through celite pad and washed with 10% MEOH in DCM and filtrate is collected and concentrated under reduced vacuum pressure to give crude. The crude material was washed with DCM to remove ammonium chloride which remains as solid and desired compound is soluble in DCM is collected and concentrated under reduced vacuum pressure to give 3-((1-(2,2,2-trifluoroethyl)azetidin-3-yl)oxy)-1-((2-(trimethylsilyl)ethoxy)methyl)-1H-pyrazol-4-amine (330.0 mg, 54%) as light pink solid. LC-MS m/z [M+H]⁺=366.53

As per the method described in Example 2, coupling to Intermediate 1 and deprotection of SEM provided for

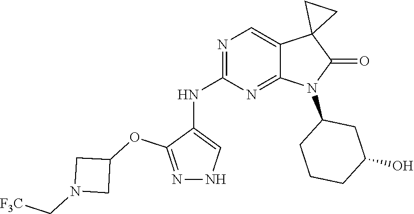 [SMILES: O=C1N([C@@H]2CCC[C@@H](O)C2)c2nc(Nc3c[nH]nc3OC3CN(CC(F)(F)F)C3)ncc2C12CC2]

7′-((1R,3R)-3-hydroxycyclohexyl)-2′-((3-((1-(2,2,2-trifluoroethyl)azetidin-3-yl)oxy)-1H-pyrazol-4-yl)amino)spiro[cyclopropane-1,5′-pyrrolo[2,3-d]pyrimidin]-6′(7′H)-one

### Example 47

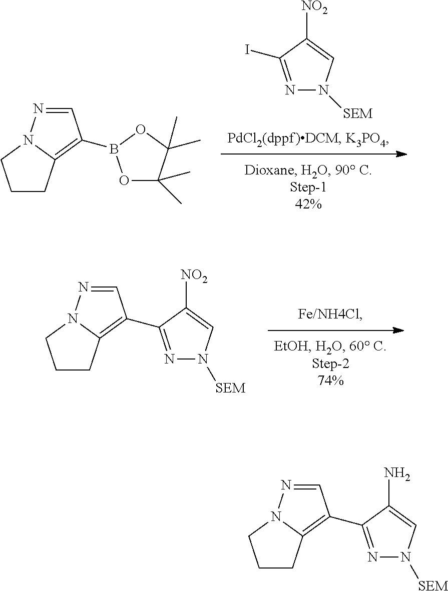

3-(4-nitro-1-((2-(trimethylsilyl)ethoxy)methyl)-1H-pyrazol-3-yl)-5,6-dihydro-4H-pyrrolo[1,2-b]pyrazole: To stirred solution of 3-iodo-4-nitro-1-((2-(trimethylsilyl)ethoxy)methyl)-1H-pyrazole (500.00 mg, 1.3542 mmol) in 1,4-Dioxane (4.500 mL), Water (0.500 mL) and degassed with nitrogen and Potassium phosphate, tribasic (718.59 mg, 3.3854 mmol) and [1,1-Bis(diphenylphosphino)ferrocene]dichloropalladium(II) Complex With Dichloromethane (110.59 mg, 135.42 μmol), 3-(4,4,5,5-tetramethyl-1,3,2-dioxaborolan-2-yl)-5,6-dihydro-4H-pyrrolo[1,2-b]pyrazole (475.54 mg, 2.0313 mmol), Then stirred the reaction mixture for 16 hours at 90° C. Reaction progress was monitored by LC-MS. LC-MS showed desired product formation. Reaction mixture was filtered through celite pad and washed with 10% MEOH in DCM and filtrate is collected and concentrated under reduced vacuum pressure to give crude product. The crude material was absorbed onto a plug of silica gel and purified by sepabean chromatography through a Redi-Sep pre-packed silica gel column (12 g), eluting with a gradient of 0-30% EtOAc in pet ether, collected the pure fractions and concentrated under reduced vacuum pressure to give 3-(4-nitro-1-((2-(trimethylsilyl)ethoxy)methyl)-1H-pyrazol-3-yl)-5,6-dihydro-4H-pyrrolo[1,2-b]pyrazole (250.0 mg, 42%) as brown gummy liquid. LC-MS m/z [M+H]+=350.24

3-(5,6-dihydro-4H-pyrrolo[1,2-b]pyrazol-3-yl)-1-((2-(trimethylsilyl)ethoxy)methyl)-1H-pyrazol-4-amine: To stirred solution of 3-(4-nitro-1-((2-(trimethylsilyl)ethoxy)methyl)-1H-pyrazol-3-yl)-5,6-dihydro-4H-pyrrolo[1,2-b]pyrazole (250.0 mg, 715.4 μmol) was taken in Ethanol (2.500 mL) and Water (1.250 mL) and Ammonium chloride (191.3 mg, 3.577 mmol) and Iron (199.8 mg, 3.577 mmol) was added. Then stirred the reaction mixture for 3 hour at 50° C. Reaction progress was monitored by LC-MS and TLC. LC-MS showed desired product formation. Reaction mixture was filtered through celite pad and washed with 10% MEOH in DCM and filtrate is collected and concentrated under reduced vacuum pressure to give crude product. The crude material was washed with DCM to remove ammonium chloride which remains as solid and desired compound is soluble in DCM is collected and concentrated under reduced vacuum pressure to give 3-(5,6-dihydro-4H-pyrrolo[1,2-b]pyrazol-3-yl)-1-((2-(trimethylsilyl)ethoxy)methyl)-1H-pyrazol-4-amine (170.0 mg, 74.38%) as brown gummy solid. LC-MS m/z [M+H]⁺=320.22

As per the method described in Example 2, coupling to Intermediate 1 and deprotection of SEM provided for

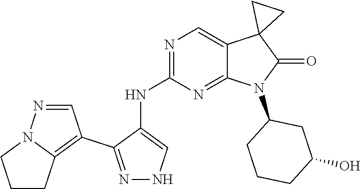 [SMILES: O=C1N([C@@H]2CCC[C@@H](O)C2)c2nc(Nc3c[nH]nc3-c3cnn4c3CCC4)ncc2C12CC2]

2′-((3-(5,6-dihydro-4H-pyrrolo[1,2-b]pyrazol-3-yl)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

### Example 48

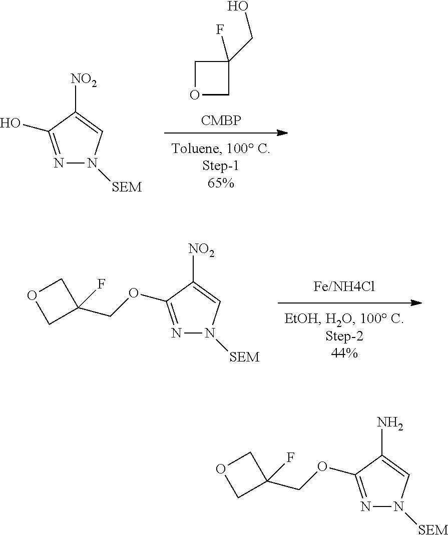

3-((3-fluorooxetan-3-yl)methoxy)-4-nitro-1-((2-(trimethylsilyl)ethoxy)methyl)-1H-pyrazole: To the stirred solution of 4-nitro-1-((2-(trimethylsilyl)ethoxy)methyl)-1H-pyrazol-3-ol (500.0 mg, 1.928 mmol) in Toluene (15.00 mL), CMBP (930.7 mg, 1.02 mL, 3.856 mmol) and (3-fluorooxetan-3-yl)methanol (306.8 mg, 2.892 mmol) is added, Then stirred the reaction mixture for 5 hours at 100° C. Reaction progress was monitored by TLC and LC-MS. TLC and LC-MS shows desired product formation. Reaction mixture was quenched with NaHCO₃ (25 ml) and extracted with ethyl acetate (2×50 ml), combined the organic layers and dried over sodium sulphate filtered and concentrated under reduced vacuum pressure to give crude product. The crude material was absorbed onto a plug of silica gel and purified by sepabean chromatography through a Redi-Sep pre-packed silica gel column (12 g), eluting with a gradient of 21.2% EtOAc in pet ether, collected the pure fractions and concentrated under reduced vacuum pressure to give 3-((3-fluorooxetan-3-yl)methoxy)-4-nitro-1-((2-(trimethylsilyl)ethoxy)methyl)-1H-pyrazole (550.0 mg, 65%) as light yellow gummy solid. LC-MS m/z [M+H]+=348.42

3-((3-fluorooxetan-3-yl)methoxy)-1-((2-(trimethylsilyl)ethoxy)methyl)-1H-pyrazol-4-amine: To s stirred solution of 3-((3-fluorooxetan-3-yl)methoxy)-4-nitro-1-((2-(trimethylsilyl)ethoxy)methyl)-1H-pyrazole (550.0 mg, 1.583 mmol) in EtOH (5.500 mL) and Water (5.500 mL) and Ammonium chloride (423.4 mg, 7.915 mmol) and Iron (442.1 mg, 7.915 mmol) was added. Then stirred the reaction mixture for 3 hours at 50° C. Reaction progress was monitored by LC-MS and TLC. LC-MS showed desired product formation. Reaction mixture was filtered through celite pad and washed with 10% MEOH in DCM and filtrate is collected and concentrated under reduced vacuum pressure to give crude product. The crude material was washed with DCM to remove ammonium chloride which remains as solid and desired compound is soluble in DCM is collected and concentrated under reduced vacuum pressure to give 3-((3-fluorooxetan-3-yl)methoxy)-1-((2-(trimethylsilyl)ethoxy)methyl)-1H-pyrazol-4-amine (400.0 mg, 44%) as brown gummy solid. LC-MS m/z [M+H]+=318.42

As per the method described in Example 2, coupling to Intermediate 1 and deprotection of SEM provided for

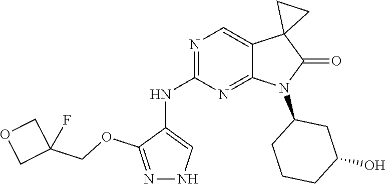 [SMILES: O=C1N([C@@H]2CCC[C@@H](O)C2)c2nc(Nc3c[nH]nc3OCC3(F)COC3)ncc2C12CC2]

2′-((3-((3-fluorooxetan-3-yl)methoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

### Example 49

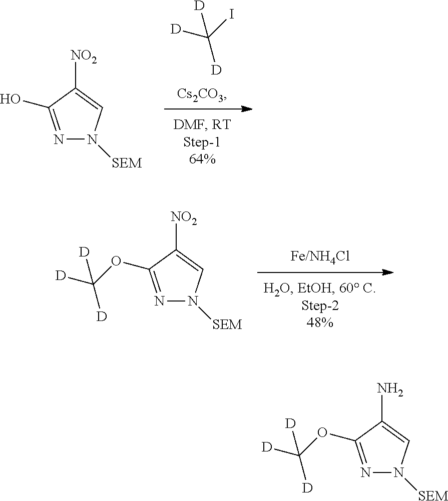

3-(methoxy-d3)-4-nitro-1-((2-(trimethylsilyl)ethoxy)methyl)-1H-pyrazole: To a stirred solution of 4-nitro-1-((2-(trimethylsilyl)ethoxy)methyl)-1H-pyrazol-3-ol (600.0 mg, 2.314 mmol) in DMF (6.000 mL) was added Cesium carbonate (1.885 g, 5.784 mmol) and iodomethane-d3 (670.7 mg, 4.627 mmol), Then stirred the reaction mixture for 3 hours at RT. Reaction progress was monitored by LC-MS. LC-MS showed desired product formation. Reaction mixture was quenched with cold water (100 ml) and filtered through Buchner funnel and washed with DCM to afford 3-(methoxy-d3)-4-nitro-1-((2-(trimethylsilyl)ethoxy)methyl)-1H-pyrazole (550.0 mg, 64.93%) as brown solid. LC-MS m/z [M+H]+=277.38

3-(methoxy-d3)-1-((2-(trimethylsilyl)ethoxy)methyl)-1H-pyrazol-4-amine: To a stirred solution of 3-(methoxy-d3)-4-nitro-1-((2-(trimethylsilyl)ethoxy)methyl)-1H-pyrazole (500.0 mg, 1.809 mmol) in Ethanol (5.000 mL) and Water (5.000 mL) and was added Ammonium chloride (483.8 mg, 9.046 mmol) and Iron (505.2 mg, 9.046 mmol). Then stirred the reaction mixture for 3 hours at 50° C. Reaction progress was monitored by LC-MS and TLC. LC-MS showed desired product formation. Reaction mixture was filtered through celite pad washed with 10% MEOH in DCM and filtrate is collected and concentrated under reduced vacuum pressure to give crude product. The crude material was washed with DCM to remove ammonium chloride which remains as solid and desired compound is soluble in DCM is collected and concentrated under reduced vacuum pressure to give. 3-(methoxy-d3)-1-((2-(trimethylsilyl)ethoxy)methyl)-1H-pyrazol-4-amine (300.0 mg, 43.29%) as brown gummy solid. LC-MS m/z [M+H]+=247.38

As per the method described in Example 2, coupling to Intermediate 1 and deprotection of SEM provided for

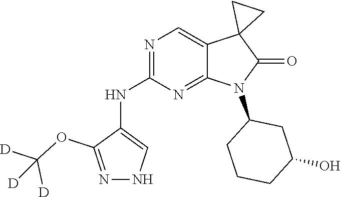 [SMILES: [2H]C([2H])([2H])Oc1n[nH]cc1Nc1ncc2c(n1)N([C@@H]1CCC[C@@H](O)C1)C(=O)C21CC1]

7′-((1R,3R)-3-hydroxycyclohexyl)-2′-((3-(methoxy-d3)-1H-pyrazol-4-yl)amino)spiro[cyclopropane-1,5′-pyrrolo[2,3-d]pyrimidin]-6′(7′H)-one

### Example 50

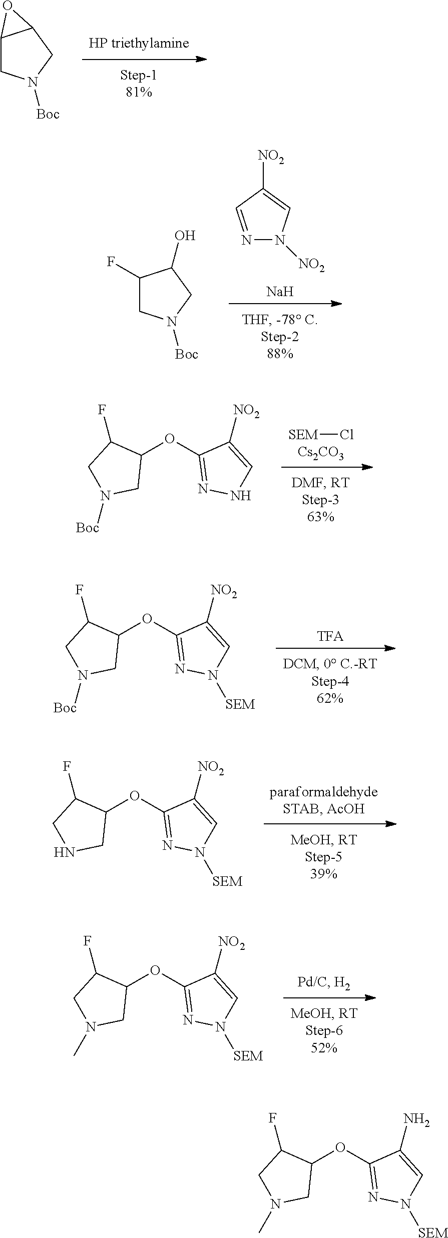

tert-butyl 3-fluoro-4-hydroxypyrrolidine-1-carboxylate: To stirred solution of tert-butyl 6-oxa-3-aza-bicyclo[3.1.0]hexane-3-carboxylate (4.000 g, 21.60 mmol) in Triethyl ammonium Fluoride (5.222 g, 5.3 mL, 32.39 mmol), Then stirred the reaction mixture for 16 hour at 120° C. Reaction progress was monitored by TLC. TLC showed desired product formation. Reaction mixture was quenched with Sat. NAHCO3 solution (125 ml) and stirred for 1 hr. and extracted with 20% IPA in DCM (2×200 mL), combined the organic layers and dried over sodium sulphate filtered and concentrated under reduced vacuum pressure to give tert-butyl 3-fluoro-4-hydroxypyrrolidine-1-carboxylate (3.600 g, 81.22%) as black gummy liquid. LC-MS m/z [M+H]+=414.53

tert-butyl 3-fluoro-4-((4-nitro-1H-pyrazol-3-yl)oxy)pyrrolidine-1-carboxylate: To stirred solution of tert-butyl 3-fluoro-4-hydroxypyrrolidine-1-carboxylate (3.500 g, 17.05 mmol) in THE (30 ml) and Sodium hydride (818.6 mg, 0.68 mL, 20.46 mmol) was added at 0° C. and in another RB flask. 1,4-dinitro-1H-pyrazole (2.696 g, 17.05 mmol) is dissolved in 90 ml THE at −78° C., then the alkoxide solution is transferred directly to SM-2 RB flask and then stirred the reaction mixture for 1 hour at −78° C. Reaction progress was monitored by TLC and LC-MS. LC-MS showed desired product formation. Reaction mixture was quenched with Sat. NH₄Cl soln. (50 ml) and extracted with ethyl acetate (2×100 ml), combined the organic layers and dried over sodium sulphate filtered and concentrated under reduced vacuum pressure to give crude product. The crude material was absorbed onto a plug of silica gel and purified by sepabean chromatography through a Redi-Sep pre-packed silica gel column (12 g), eluting with a gradient of 28-32% EtOAc in pet ether, collected the pure fractions and concentrated under reduced vacuum pressure to give tert-butyl 3-fluoro-4-((4-nitro-1H-pyrazol-3-yl)oxy)pyrrolidine-1-carboxylate (1.500 g, 424%) as light yellow liquid. LC-MS m/z [M+H]+=261.52 (M-56)

tert-butyl 3-fluoro-4-((4-nitro-1-((2-(trimethylsilyl)ethoxy)methyl)-1H-pyrazol-3-yl)oxy)pyrrolidine-1-carboxylate: To stirred solution of tert-butyl 3-fluoro-4-((4-nitro-1H-pyrazol-3-yl)oxy)pyrrolidine-1-carboxylate (1.500 g, 4.742 mmol) in DMF (7.000 mL), Cesium carbonate (3.090 g, 9.485 mmol) is added at 0° C. and 2-(Chloromethoxyethyl)trimethyl silane (1.186 g, 1.26 mL, 7.114 mmol) was added Then stirred the reaction mixture for 1 hour at RT, Reaction progress was monitored by TLC and LC-MS. LC-MS showed desired product formation. Reaction mixture was quenched with cold water (50 ml) and extracted with ethyl acetate (2×100 ml), combined the organic layers and dried over sodium sulphate, filtered and concentrated under reduced vacuum pressure to give crude product. The crude material was absorbed onto a plug of silica gel and purified by sepabean chromatography through a Redi-Sep pre-packed silica gel column (40 g), eluting with a gradient of 25-28% EtOAc in pet ether, collected the pure fractions and concentrated under reduced vacuum pressure to give tert-butyl 3-fluoro-4-((4-nitro-1-((2-(trimethylsilyl)ethoxy)methyl)-1H-pyrazol-3-yl)oxy)pyrrolidine-1-carboxylate (1.500 g, 363%) as light yellow gummy solid. LC-MS m/z [M+H]⁺=447.24

3-((4-fluoropyrrolidin-3-yl)oxy)-4-nitro-1-((2-(trimethylsilyl)ethoxy)methyl)-1H-pyrazole: To stirred solution of 3-fluoro-4-((4-nitro-1-((2-(trimethylsilyl)ethoxy)methyl)-1H-pyrazol-3-yl)oxy)pyrrolidine-1-carboxylate (1.500 g, 3.359 mmol) in DCM (15.00 mL) and TFA (296 mg, 0.200 mL, 2.60 mmol) was added and then stirred the reaction mixture for 1 hour at RT, Reaction progress was monitored by TLC and LC-MS. LC-MS showed desired product formation. Reaction mixture was quenched with Cold sat. NaHCO₃ solution (100 ml) and extracted with 10% MEOH IN DCM (2×100 ml), combined the organic layers and dried over sodium sulphate filtered and concentrated under reduced vacuum pressure to give 3-((4-fluoropyrrolidin-3-yl)oxy)-4-nitro-1-((2-(trimethylsilyl)ethoxy)methyl)-1H-pyrazole (900.0 mg, 62%, 80% Purity) as light yellow gummy solid. LC-MS m/z [M+H]⁺=347.53

3-((4-fluoro-1-methylpyrrolidin-3-yl)oxy)-4-nitro-1-((2-(trimethylsilyl)ethoxy)methyl)-1H-pyrazole: To a stirred solution of 3-((4-fluoropyrrolidin-3-yl)oxy)-4-nitro-1-((2-(trimethylsilyl)ethoxy)methyl)-1H-pyrazole (900.00 mg, 2.5979 mmol) in Methanol (18.000 mL), Paraformaldehyde (780.2 mg, 25.979 mmol), Acetic acid (156.0 mg, 149.3 μL, 2.5979 mmol) is added at 0° C. and after 1 hour Sodium triacetoxyborohydride (1.6518 g, 7.7938 mmol) was added, then stirred the reaction mixture for 16 hours at RT, Reaction progress was monitored by LC-MS. LC-MS showed desired product formation. Reaction mixture was quenched with water (50 ml) and extracted with DCM (2×100 ml), combined the organic layers and dried over sodium sulphate filtered and concentrated under reduced vacuum pressure to give crude product. The crude material was absorbed onto a plug of silica gel and purified by sepabean chromatography through a Redi-Sep pre-packed silica gel column (12 g), eluting with a gradient of 45-55% EtOAc in pet ether, collected the pure fractions and concentrated under reduced vacuum pressure to give desired product 3-((4-fluoro-1-methylpyrrolidin-3-yl)oxy)-4-nitro-1-((2-(trimethylsilyl)ethoxy)methyl)-1H-pyrazole (270.0 mg, 39%, 87% Purity) LC-MS m/z (M+H)+: 361.24

3-((4-fluoro-1-methylpyrrolidin-3-yl)oxy)-1-((2-(trimethylsilyl)ethoxy)methyl)-1H-pyrazol-4-amine: To stirred solution of 3-((4-fluoro-1-methylpyrrolidin-3-yl)oxy)-4-nitro-1-((2-(trimethylsilyl)ethoxy)methyl)-1H-pyrazole (200.00 mg, 554.85 μmol) has taken in Methanol (2.000 mL) and Pd/C (118.09 mg, 110.97 μmol) was added and H₂ gas is passed through bladder at 5 psi pressure, then stirred the reaction mixture for 2 hours at RT Reaction progress was monitored by TLC and LC-MS and TLC. TLC and LC-MS showed desired product formation. Reaction mixture was filtered through celite pad and washed with 10% MEOH in DCM and filtrate is collected and concentrated under reduced vacuum pressure to give 3-((4-fluoro-1-methylpyrrolidin-3-yl)oxy)-1-((2-(trimethylsilyl)ethoxy)methyl)-1H-pyrazol-4-amine (170.0 mg, 52%) as light brown gummy solid. LC-MS m/z [M+H]+=331.24

As per the method described in Example 2, coupling to Intermediate 1 and deprotection of SEM followed by chiral SFC provided for Peak 1

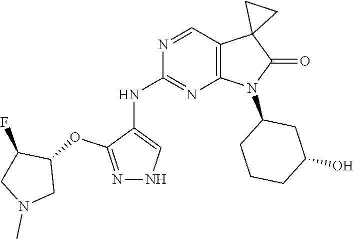 [SMILES: CN1C[C@@H](F)[C@H](Oc2n[nH]cc2Nc2ncc3c(n2)N([C@@H]2CCC[C@@H](O)C2)C(=O)C32CC2)C1]

2′-((3-(((3R,4R)-4-fluoro-1-methylpyrrolidin-3-yl)oxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

### Example 51

Peak 2 from chiral SFC in example 50 provided for

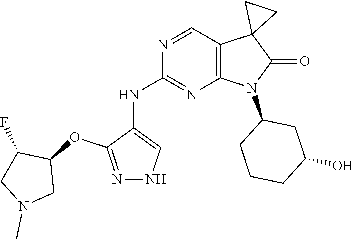 [SMILES: CN1C[C@H](Oc2n[nH]cc2Nc2ncc3c(n2)N([C@@H]2CCC[C@@H](O)C2)C(=O)C32CC2)[C@@H](F)C1]

2′-((3-(((3S,4S)-4-fluoro-1-methylpyrrolidin-3-yl)oxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

### Example 52

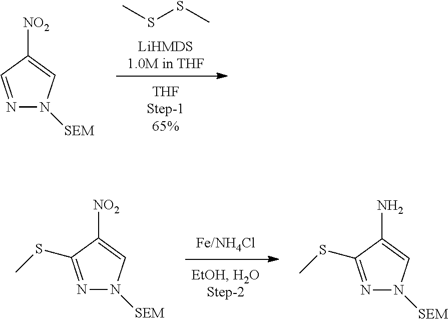

3-(methylthio)-4-nitro-1-((2-(trimethylsilyl)ethoxy)methyl)-1H-pyrazole: To the stirred solution of 4-nitro-1-((2-(trimethylsilyl)ethoxy)methyl)-1H-pyrazole (1 g, 4.11 mmol) in THE (20.00 mL) LiHMDS 1.0 M in THE (6.16 mL, 6.16 mmol) was added at −78° C. and 1,2-dimethyldisulfane (580.6 mg, 6.16 mmol) is added, Then stirred the reaction mixture for 2 hour at RT. Reaction progress was monitored by TLC and LC-MS. TLC and LC-MS shows desired product formation. Reaction mixture was quenched with NH₄Cl (25 ml) and extracted with ethyl acetate (2×50 ml), combined the organic layers and dried over sodium sulphate filtered and concentrated under reduced vacuum pressure to give 3-(methylthio)-4-nitro-1-((2-(trimethylsilyl)ethoxy)methyl)-1H-pyrazole (550.0 mg) as light yellow gummy solid. LC-MS m/z [M+H]+=289.42

3-(methylthio)-1-((2-(trimethylsilyl)ethoxy)methyl)-1H-pyrazol-4-amine: To a stirred solution 3-(methylthio)-4-nitro-1-((2-(trimethylsilyl)ethoxy)methyl)-1H-pyrazole (300.0 mg, 1.04 mmol) in EtOH (3.00 mL) and Water (3.00 mL) and Ammonium chloride (277 mg, 5.18 mmol) and Iron (289 mg, 5.18 mmol) was added. Then stirred the reaction mixture for 3 hours at 50° C. Reaction progress was monitored by LC-MS and TLC. LC-MS showed desired product formation. Reaction mixture was filtered through celite pad and washed with 10% MEOH in DCM and filtrate is collected and concentrated under reduced vacuum pressure to give crude product. The crude material was washed with DCM to remove ammonium chloride which remains as solid and desired compound is soluble in DCM is collected and concentrated under reduced vacuum pressure to 3-(methylthio)-1-((2-(trimethylsilyl)ethoxy)methyl)-1H-pyrazol-4-amine (270.0 mg) as Yellowish brown gummy solid. LC-MS m/z (M+H)+: 259.42

As per the method described in Example 2, coupling to Intermediate 1 and deprotection of SEM provided for

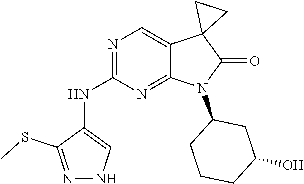 [SMILES: CSc1n[nH]cc1Nc1ncc2c(n1)N([C@@H]1CCC[C@@H](O)C1)C(=O)C21CC1]

7′-((1R,3R)-3-hydroxycyclohexyl)-2′-((3-(methylthio)-1H-pyrazol-4-yl)amino)spiro[cyclopropane-1,5′-pyrrolo[2,3-d]pyrimidin]-6′(7′H)-one

### Example 53

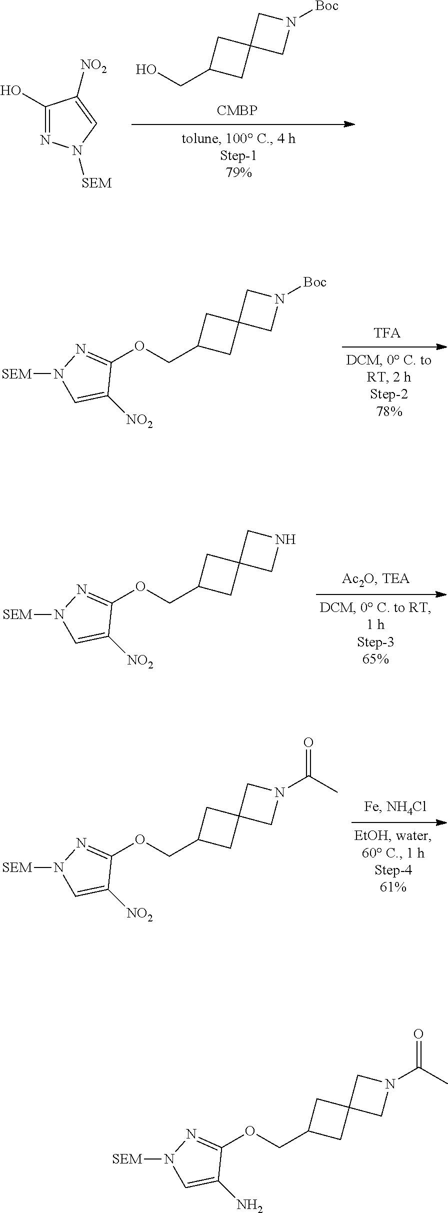 [SMILES: C[Si](C)(C)CCOCn1cc([N+](=O)[O-])c(O)n1 ; CC(C)(C)OC(=O)N1CC2(CC(CO)C2)C1 ; CC(C)(C)OC(=O)N1CC2(CC(COc3nn(COCC[Si](C)(C)C)cc3[N+](=O)[O-])C2)C1 ; C[Si](C)(C)CCOCn1cc([N+](=O)[O-])c(OCC2CC3(CNC3)C2)n1 ; CC(=O)N1CC2(CC(COc3nn(COCC[Si](C)(C)C)cc3[N+](=O)[O-])C2)C1 ; CC(=O)N1CC2(CC(COc3nn(COCC[Si](C)(C)C)cc3N)C2)C1]

tert-butyl 6-(((4-nitro-1-((2-(trimethylsilyl)ethoxy)methyl)-1H-pyrazol-3-yl)oxy)methyl)-2-azaspiro[3.3]heptane-2-carboxylate: To a stirred solution of 4-nitro-1-((2-(trimethylsilyl)ethoxy)methyl)-1H-pyrazol-3-ol (800.0 mg, 3.085 mmol) in toluene (1 mL) was added tert-butyl 6-(hydroxymethyl)-2-azaspiro[3.3]heptane-2-carboxylate (1.052 g, 4.627 mmol) followed by CMBP (1.65 mL, 1.5 Eq, 4.627 mmol) at RT and the resulting reaction mixture was stirred at 100° C. for 4 hour. The reaction progress was monitored by TLC and LCMS. On completion, the reaction mixture was diluted with water (10 mL) and extracted with ethyl acetate (2×20 mL). Combined organic layers was washed with brine (5 mL), dried over sodium sulphate, filtered and evaporated under reduced vacuum pressure to give crude product. The crude material was absorbed onto a plug of silica gel and purified by SepaBean chromatography through a RediSep pre-packed silica gel column (24 g), eluting with a gradient of 0-30% EtOAc in pet ether, collected the pure fractions and concentrated under reduced vacuum pressure to afford tert-butyl 6-(((4-nitro-1-((2-(trimethylsilyl)ethoxy)methyl)-1H-pyrazol-3-yl)oxy)methyl)-2-azaspiro[3.3]heptane-2-carboxylate (1.2 g, 79.07% yield) as a pale yellow oil. LC-MS m/z [M+H]⁺=469.54

6-(((4-nitro-1-((2-(trimethylsilyl)ethoxy)methyl)-1H-pyrazol-3-yl)oxy)methyl)-2-azaspiro[3.3]heptane: To a stirred solution of tert-butyl 6-(((4-nitro-1-((2-(trimethylsilyl)ethoxy)methyl)-1H-pyrazol-3-yl)oxy)methyl)-2-azaspiro[3.3]heptane-2-carboxylate (600 mg, 1.2803 mmol) in DCM (12 mL) was added TFA (1.2 mL) at 0° C. and the resulting reaction mixture was stirred at RT for 2 hours. The reaction progress was monitored by TLC and LCMS. On completion, the reaction mixture was quenched with sat. NaHCO₃ solution (10 mL) and extracted with DCM (2×20 mL). The combined organic layer was washed with brine (10 mL), dried over sodium sulphate, filtered and evaporated under reduced vacuum pressure to give 6-(((4-nitro-1-((2-(trimethylsilyl)ethoxy)methyl)-1H-pyrazol-3-yl)oxy)methyl)-2-azaspiro[3.3]heptane (450 mg, 78.04% yield) as a yellow oil. LC-MS m/z [M+H]⁺=369.45

1-(6-(((4-nitro-1-((2-(trimethylsilyl)ethoxy)methyl)-1H-pyrazol-3-yl)oxy)methyl)-2-azaspiro[3.3]heptan-2-yl)ethan-1-one: To a stirred solution of 6-(((4-nitro-1-((2-(trimethylsilyl)ethoxy)methyl)-1H-pyrazol-3-yl)oxy)methyl)-2-azaspiro[3.3]heptane (500 mg, 1.3568 mmol) in DCM (7 mL) was added TEA (0.6 mL, 4.07 mmol) followed by acetic anhydride (0.2 mL, 1.5 Eq, 2.0352 mmol) at 0° C. and the resulting reaction mixture was stirred at RT for 1 hour. The reaction progress was monitored by TLC and LCMS. On completion, the reaction mixture was diluted with water (10 mL) and extracted with DCM (2×20 mL). The combined the organic layer was washed with brine (5 mL), dried over sodium sulphate, filtered and concentrated under reduced vacuum pressure to afford 1-(6-(((4-nitro-1-((2-(trimethylsilyl)ethoxy)methyl)-1H-pyrazol-3-yl)oxy)methyl)-2-azaspiro[3.3]heptan-2-yl)ethan-1-one (450 mg, 65.44% yield) as a yellow oil. LC-MS m/z [M+H]⁺=411.47

1-(6-(((4-amino-1-((2-(trimethylsilyl)ethoxy)methyl)-1H-pyrazol-3-yl)oxy)methyl)-2-azaspiro[3.3]heptan-2-yl)ethan-1-one: To a stirred solution of 1-(6-(((4-nitro-1-((2-(trimethylsilyl)ethoxy)methyl)-1H-pyrazol-3-yl)oxy)methyl)-2-azaspiro[3.3]heptan-2-yl)ethan-1-one (450 mg, 1.0961 mmol) in ethanol (4.5 mL) and water (4.5 mL) was added ammonium chloride (295 mg, 5.4805 mmol) followed by iron (306 mg, 5.4805 mmol) at RT and the resulting reaction mixture was stirred at 60° C. for 1 hour. The reaction progress was monitored by TLC and LCMS. On completion, the reaction mixture was passed through celite pad and pad was washed with 10% MeOH in DCM (5 mL). Filtrate was diluted with water (5 mL), extracted with DCM (2×10 mL), washed with brine (5 mL), dried over sodium sulfate and evaporated under reduced vacuum pressure to afford 1-(6-(((4-amino-1-((2-(trimethylsilyl)ethoxy)methyl)-1H-pyrazol-3-yl)oxy)methyl)-2-azaspiro[3.3]heptan-2-yl)ethan-1-one (400 mg, 61.22% yield) as a brown oil. LC-MS m/z [M+H]⁺=381.54

As per the method described in Example 2, coupling to Intermediate 1 and deprotection of SEM provided for

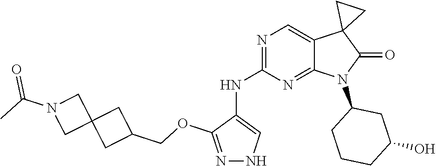 [SMILES: CC(=O)N1CC2(CC(COc3n[nH]cc3Nc3ncc4c(n3)N([C@@H]3CCC[C@@H](O)C3)C(=O)C43CC3)C2)C1]

2′-((3-((2-acetyl-2-azaspiro[3.3]heptan-6-yl)methoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

### Example 54

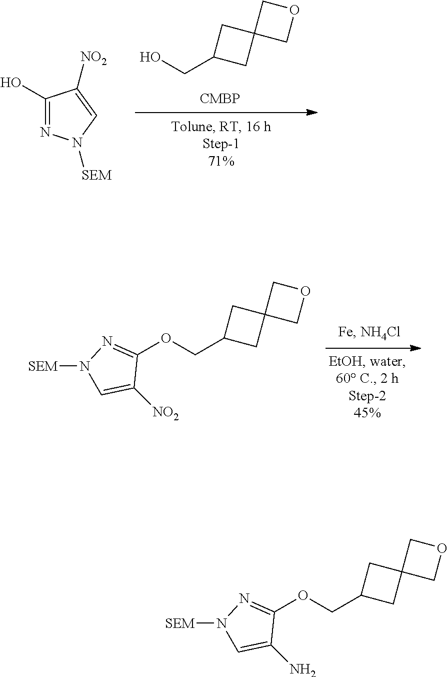 [SMILES: C[Si](C)(C)CCOCn1cc([N+](=O)[O-])c(O)n1 ; OCC1CC2(COC2)C1 ; C[Si](C)(C)CCOCn1cc([N+](=O)[O-])c(OCC2CC3(COC3)C2)n1 ; C[Si](C)(C)CCOCn1cc(N)c(OCC2CC3(COC3)C2)n1]

3-((2-oxaspiro[3.3]heptan-6-yl)methoxy)-4-nitro-1-((2-(trimethylsilyl)ethoxy)methyl)-1H-pyrazole: To a stirred solution of 4-nitro-1-((2-(trimethylsilyl)ethoxy)methyl)-1H-pyrazol-3-ol (400 mg, 1.5424 mmol) in toluene (4.0 mL) was added (2-oxaspiro[3.3]heptan-6-yl)methanol (336 mg, 2.6220 mmol) followed by CMBP (0.8 mL, 3.0848 mmol) and the resulting reaction mixture was stirred at RT for 16 hour. The reaction progress was monitored by TLC and LCMS. On completion, the reaction mixture was diluted with water (5 ml) and extracted with ethyl acetate (2×10 mL). The combined organic layer was washed with brine (5 mL), dried over sodium sulphate, filtered and concentrated under reduced vacuum pressure to give crude product. The crude material was absorbed onto a plug of silica gel and purified by Sepabean chromatography through a RediSep pre-packed silica gel column (24 g), eluting with a gradient of 0-50% EtOAc in pet ether, collected the pure fractions and concentrated under reduced vacuum pressure to afford Compound-3 (480 mg, 71.11% yield) as a pale yellow oil. LC-MS m/z [M+H]⁺=370.41

3-((2-oxaspiro[3.3]heptan-6-yl)methoxy)-1-((2-(trimethylsilyl)ethoxy)methyl)-1H-pyrazol-4-amine: To a stirred solution of 3-((2-oxaspiro[3.3]heptan-6-yl)methoxy)-4-nitro-1-((2-(trimethylsilyl)ethoxy)methyl)-1H-pyrazole (480 mg, 1.299 mmol) in ethanol (5 mL) and water (5 mL) was added ammonium chloride (345 mg, 6.495 mmol) followed by iron (363 mg, 6.495 mmol) at RT and the resulting reaction mixture was stirred at 60° C. for 2 hours. The reaction progress was monitored by TLC and LCMS. After completion of reaction, all volatiles were evaporated in vacuo, the residue obtained was dissolved in dichloromethane (10 mL), filtered and filtrate was evaporated in vacuum to afford 3-((2-oxaspiro[3.3]heptan-6-yl)methoxy)-1-((2-(trimethylsilyl)ethoxy)methyl)-1H-pyrazol-4-amine (400 mg, 44.70% yield. LC-MS m/z [M+H]⁺=340.43

As per the method described in Example 2, coupling to Intermediate 1 and deprotection of SEM provided for

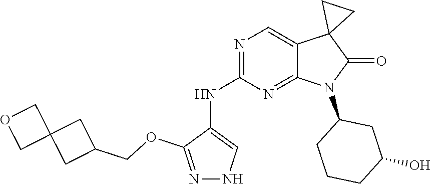 [SMILES: O=C1N([C@@H]2CCC[C@@H](O)C2)c2nc(Nc3c[nH]nc3OCC3CC4(COC4)C3)ncc2C12CC2]

2′-((3-((2-oxaspiro[3.3]heptan-6-yl)methoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

### Example 55

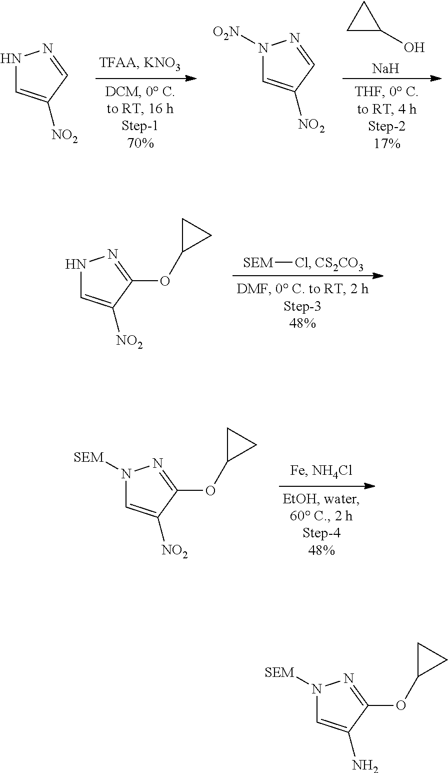

1,4-dinitro-1H-pyrazole: To a stirred solution of 4-nitro-1H-pyrazole (2.5 g, 22.11 mmol) in DCM (25 mL) was added potassium nitrate (6.7 g, 66.32 mmol) followed by TFAA (12.5 mL, 88.44 mmol) at 0° C. and the resulting reaction mixture was stirred at RT for 16 hours. The reaction progress was monitored by TLC and LCMS. On completion, the reaction mixture was cooled to 0° C., quenched with water (50 mL) and extracted with DCM (2×25 mL). The combined organic layer was washed with brine (15 mL), dried over Na₂SO₄ and evaporated under reduced pressure to afford 1,4-dinitro-1H-pyrazole (2.5 g, 70.10% yield). ¹H NMR (400 MHz, CdCl₃) δ=9.035 (d, 8.0 Hz), 8.182 (d, 8.0 Hz).

3-cyclopropoxy-4-nitro-1H-pyrazole: To a stirred solution of 1,4-dinitro-1H-pyrazole (0.62 mL, 9.75 mmol) in THE (2.3 mL) was added NaH (1.3 g, 31.63 mmol) at 0° C., stirred for 10 minutes. This suspension was added in the stirred solution of cyclopropanol (2.3 g, 14.55 mmol) in THE (2.3 mL) at −78° C. and the resulting reaction mixture was stirred at RT for 1 hour. The reaction progress was monitored by TLC and LCMS. The reaction mixture was diluted cold water (20 mL) and extracted with EtOAc (2×20 mL), dried over sodium sulfate, evaporated under reduced pressure to afford crude. The crude material was absorbed onto a plug of silica gel and purified by Sepabean chromatography through a RediSep pre-packed silica gel column (24 g), eluting with a gradient of 0-70% EtOAc in pet ether, collected the pure fractions and concentrated under reduced vacuum pressure to afford 3-cyclopropoxy-4-nitro-1H-pyrazole (0.55 g, 17.47% yield) as a yellow solid. LC-MS m/z [M+H]⁺=170.07.

3-cyclopropoxy-4-nitro-1-((2-(trimethylsilyl)ethoxy)methyl)-1H-pyrazole: To a stirred solution of 3-cyclopropoxy-4-nitro-1H-pyrazole (550 mg, 3.252 mmol) in N,N-dimethylformamide (5.5 mL) was added cesium carbonate (1.6 g, 4.878 mmol) followed by SEM-Cl (0.7 mL, 3.902 mmol) at 0° C. and the resulting reaction mixture was stirred at RT for 2 hour. The reaction progress was monitored by TLC and LCMS. After completion of reaction, the reaction mixture was diluted with cold water (5 mL) and extracted with EtOAc (2×20 mL). The combined organic layer was washed with brine (5 mL), dried over Na₂SO₄ and evaporated under reduced pressure to afford crude. The crude material was absorbed onto a plug of silica gel and purified by Sepabean chromatography through a RediSep pre-packed silica gel column (12 g), eluting with a gradient of 0-20% EtOAc in pet ether, collected pure fractions and concentrated under reduced vacuum pressure to afford 3-cyclopropoxy-4-nitro-1-((2-(trimethylsilyl)ethoxy)methyl)-1H-pyrazole (0.3 g, 48.14% yield) as a brown solid. LC-MS m/z [M+H]⁺=300.23.

3-cyclopropoxy-1-((2-(trimethylsilyl)ethoxy)methyl)-1H-pyrazol-4-amine: To a stirred solution of 3-cyclopropoxy-4-nitro-1-((2-(trimethylsilyl)ethoxy)methyl)-1H-pyrazole (350 mg, 1.169 mmol) in ethanol (3.5 mL) and water (3.5 mL) was added ammonium chloride (315 mg, 5.845 mmol) followed by iron (326 mg, 5.845 mmol) at RT and the resulting reaction mixture was stirred at 60° C. for 1 hour. The reaction progress was monitored by TLC and LCMS. After completion of reaction, all volatiles were evaporated in vacuo, the residue obtained was dissolved in dichloromethane (5 mL), filtered and filtrate was evaporated in vacuo to afford 3-cyclopropoxy-1-((2-(trimethylsilyl)ethoxy)methyl)-1H-pyrazol-4-amine (0.3 g, 48.14% yield) as a brown solid. LC-MS m/z [M+H]⁺=270.28

As per the method described in Example 2, coupling to Intermediate 1 and deprotection of SEM provided for

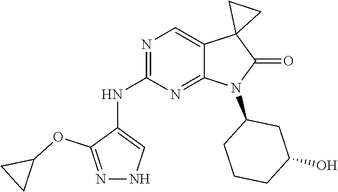 [SMILES: O=C1N([C@@H]2CCC[C@@H](O)C2)c2nc(Nc3c[nH]nc3OC3CC3)ncc2C12CC2]

2-((3-cyclopropoxy-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

### Example 56

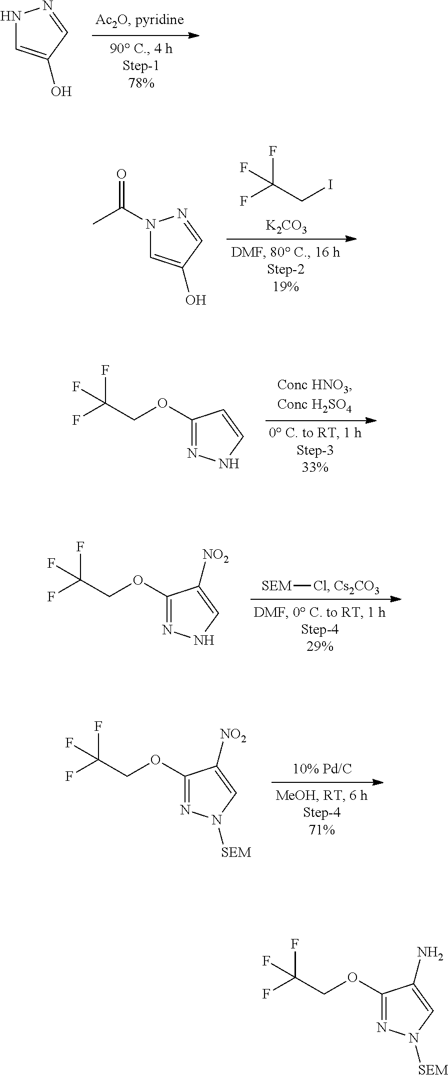

1-(4-hydroxy-1H-pyrazol-1-yl)ethan-1-one: To a stirred solution of 1H-pyrazol-4-ol (10.0 g, 118.94 mmol) in pyridine (35 mL) was added acetic anhydride (12 mL, 127.34 mmol) in pyridine (35 mL) at RT and the resulting reaction mixture was stirred at 90° C. for 4 hours. The reaction progress was monitored by TLC and LCMS. On completion, all volatiles were evaporated under reduced pressure to afford crude. The crude was washed with diethyl ether (100 mL) and filtered to obtain 1-(4-hydroxy-1H-pyrazol-1-yl)ethan-1-one (12 g, 78.21% yield) as pale yellow solid. LC-MS m/z [M+H]⁺=126.97

3-(2,2,2-trifluoroethoxy)-1H-pyrazole: To a stirred solution of 1-(4-hydroxy-1H-pyrazol-1-yl)ethan-1-one (3.0 g, 23.79 mmol) in DMF (30 mL) was added K₂CO₃ (6.6 g, 47.58 mmol) followed by trifluoromethyliodide (3.5 mL, 35.68 mmol) at RT and the resulting reaction mixture was stirred at 80° C. for 16 hours. The reaction progress was monitored by TLC and LCMS. On completion, the reaction mixture was diluted with cold water (50 mL), extracted with EtOAc (2×50 mL), washed with brine (30 mL), dried over sodium sulphate and evaporated under reduced vacuum pressure to afford 3-(2,2,2-trifluoroethoxy)-1H-pyrazole (1.5 g, 19.34% yield) as yellow solid. LC-MS m/z [M+H]⁺=167.15

4-nitro-3-(2,2,2-trifluoroethoxy)-1H-pyrazole: To a stirred solution of 3-(2,2,2-trifluoroethoxy)-1H-pyrazole (1.5 g, 9.03 mmol) in conc. H₂SO₄ (4.5 mL) was added conc. HNO₃ (1.5 mL) dropwise at 0° C. and the resulting reaction mixture was stirred at 60° C. for 1 hour. The reaction progress was monitored by TLC and LCMS. On completion, the reaction mixture was poured on crushed ice, pale yellow solid separated was filtered, washed with n-pentane (10 mL) to afford 4-nitro-3-(2,2,2-trifluoroethoxy)-1H-pyrazole (1.5 g, 33.05% yield) as pale yellow solid. LC-MS m/z [M+H]⁺=210.11

4-nitro-3-(2,2,2-trifluoroethoxy)-1-((2-(trimethylsilyl)ethoxy)methyl)-1H-pyrazole: To a stirred solution of 4-nitro-3-(2,2,2-trifluoroethoxy)-1H-pyrazole (1.5 g, 7.11 mmol) in DMF (15 mL) was added cesium carbonate (4.7 g, 10.66 mmol) followed by SEM-Cl (1.9 mL, 10.66 mmol) at 0° C. and the resulting reaction mixture was stirred at RT for 1 hour. The reaction progress was monitored by TLC and LCMS. On completion, the reaction mixture was diluted with cold water (50 mL), extracted with EtOAc (2×50 mL), washed with brine (20 mL), dried over sodium sulfate and evaporated under reduced vacuum pressure to afford crude. The crude compound was purified by SepaBean using silica gel (100-200 mesh), eluting with 0-10% ethyl acetate in pet-ether to give 4-nitro-3-(2,2,2-trifluoroethoxy)-1-((2-(trimethylsilyl)ethoxy)methyl)-1H-pyrazole (0.7 g, 29% yield) as a yellow oil. LC-MS m/z [M+H]⁺=342.55

3-(2,2,2-trifluoroethoxy)-1-((2-(trimethylsilyl)ethoxy)methyl)-1H-pyrazol-4-amine: To a stirred solution of 4-nitro-3-(2,2,2-trifluoroethoxy)-1-((2-(trimethylsilyl)ethoxy)methyl)-1H-pyrazole (300 mg, 0.878 mmol) in ethanol (2 mL) and water (3 mL) was added 10% Pd/C (90 mg, 30% w/w) and the resulting reaction mixture was stirred at RT under hydrogen balloon pressure for 6 hours. The reaction progress was monitored by TLC and LCMS. After completion of reaction, the reaction mixture was passed through celite pad and pad was washed with MeOH (10 mL). Filtrate was evaporated under reduced pressure to afford

3-(2,2,2-trifluoroethoxy)-1-((2-(trimethylsilyl)ethoxy)methyl)-1H-pyrazol-4-amine (0.3 g, 76.74% yield) as a brown solid. LC-MS m/z [M+H]⁺=312.07

As per the method described in Example 2, coupling to Intermediate 1 and deprotection of SEM provided for

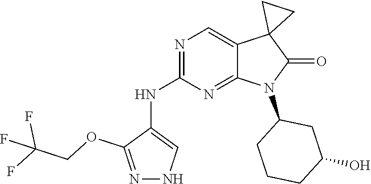 [SMILES: O=C1N([C@@H]2CCC[C@@H](O)C2)c2nc(Nc3c[nH]nc3OCC(F)(F)F)ncc2C12CC2]

7′-((1R,3R)-3-hydroxycyclohexyl)-2′-((3-(2,2,2-trifluoroethoxy)-1H-pyrazol-4-yl)amino)spiro[cyclopropane-1,5′-pyrrolo[2,3-d]pyrimidin]-6′(7′H)-one

### Example 57

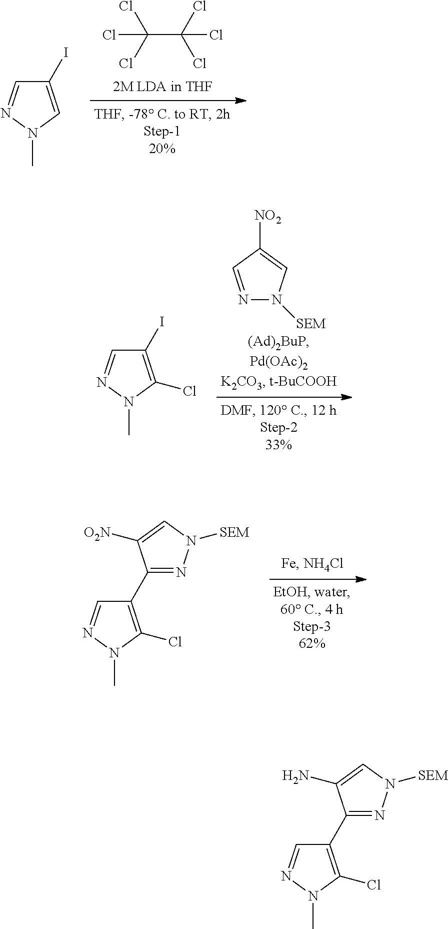

5-chloro-4-iodo-1-methyl-1H-pyrazole: To a stirred solution of 4-iodo-1-methyl-1H-pyrazole (1 g, 1 Eq, 48.08 mmol) in THE (10 mL) was added 2M LDA in THE (48 mL, 2 eq 96.2 mmol) at −78° C., stirred for 30 minutes and then was added hexachloroethane (1.4 g, 1 Eq, 57.7 mmol) and the resulting reaction mixture was stirred at RT for 2 hours. The reaction progress was monitored by TLC and LCMS. On completion, the reaction mixture was cooled to 0° C., quenched with saturated NH₄Cl solution (150 mL) and extracted with EtOAc (2×150 mL). The combined organic layer was washed with brine (100 mL), dried over Na₂SO₄ and evaporated under reduced pressure to afford crude. The crude material was absorbed onto a plug of silica gel and purified by Sepabean chromatography through a RediSep pre-packed silica gel column (120 g), eluting with a gradient of 0-30% EtOAc in pet ether, collected the pure fractions and concentrated under reduced vacuum pressure to afford 5-chloro-4-iodo-1-methyl-1H-pyrazole (3.5 g, 19.52% yield) as a yellow oil. LC-MS m/z [M+H]⁺=243.10

5′-chloro-1′-methyl-4-nitro-1-((2-(trimethylsilyl)ethoxy)methyl)-1H,1′H-3,4′-bipyrazole: To a stirred solution of 5-chloro-4-iodo-1-methyl-1H-pyrazole (1 g, 1 Eq, 4.12 mmol) in DMF (20 mL) was added 4-nitro-1-((2-(trimethylsilyl)ethoxy)methyl)-1H-pyrazole (1.5 g, 1.2 Eq, 6.19 mmol) followed by K₂CO₃ (1.43 g, 2.5 Eq, 10.31 mmol), (Ad)₂BuP (0.148 g, 0.1 Eq, 0.412 mmol), Pd(OAc)₂ (0.046 g, 0.05 Eq, 0.206 mmol) and t-BuCOOH (0.210 g, 0.05 Eq, 0.206 mmol) at RT and the resulting reaction mixture was stirred at 120° C. for 12 hours. The reaction progress was monitored by TLC and LCMS. The reaction mixture was diluted cold water (20 mL) and extracted with EtOAc (2×20 mL), dried over sodium sulfate, evaporated under reduced pressure to afford crude. The crude material was absorbed onto a plug of silica gel and purified by Sepabean chromatography through a RediSep pre-packed silica gel column (24 g), eluting with a gradient of 0-50% EtOAc in pet ether, collected the pure fractions and concentrated under reduced vacuum pressure to afford 5′-chloro-1′-methyl-4-nitro-1-((2-(trimethylsilyl)ethoxy)methyl)-1H,1′H-3,4′-bipyrazole (0.55 g, 33.33% yield) as a yellow solid. LC-MS m/z [M+H]⁺=358.47

5′-chloro-1′-methyl-1-((2-(trimethylsilyl)ethoxy)methyl)-1H,1′H-[3,4′-bipyrazol]-4-amine: To a stirred solution of 5′-chloro-1′-methyl-4-nitro-1-((2-(trimethylsilyl)ethoxy)methyl)-1H,1′H-3,4′-bipyrazole (500 mg, 1 Eq, 1.40 mmol) in ethanol (5 mL) and water (3.5 mL) was added ammonium chloride (375 mg, 5 Eq, 6.99 mmol) followed by iron (391 mg, 5 Eq, 6.99 mmol) at RT and the resulting reaction mixture was stirred at 60° C. for 4 hours. The reaction progress was monitored by TLC and LCMS. After completion of reaction, all volatiles were evaporated in vacuum, the residue obtained was dissolved in dichloromethane (15 mL), filtered and filtrate was evaporated in vacuum to afford 5′-chloro-1′-methyl-1-((2-(trimethylsilyl)ethoxy)methyl)-1H,1′H-[3,4′-bipyrazol]-4-amine (0.4 g, 61.69% yield) as a brown oil. LC-MS m/z [M+H]⁺=328.50

As per the method described in Example 2, coupling to Intermediate 1 and deprotection of SEM provided for

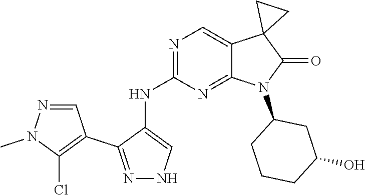 [SMILES: Cn1ncc(-c2n[nH]cc2Nc2ncc3c(n2)N([C@@H]2CCC[C@@H](O)C2)C(=O)C32CC2)c1Cl]

2′-((5′-chloro-1′-methyl-1H,1′H-[3,4′-bipyrazol]-4-yl)amino)-7-((1R,3R)-3-hydroxycyclohexyl)spiro[cyclopropane-1,5′-pyrrolo[2,3-d]pyrimidin]-6′(7′H)-one

### Example 58

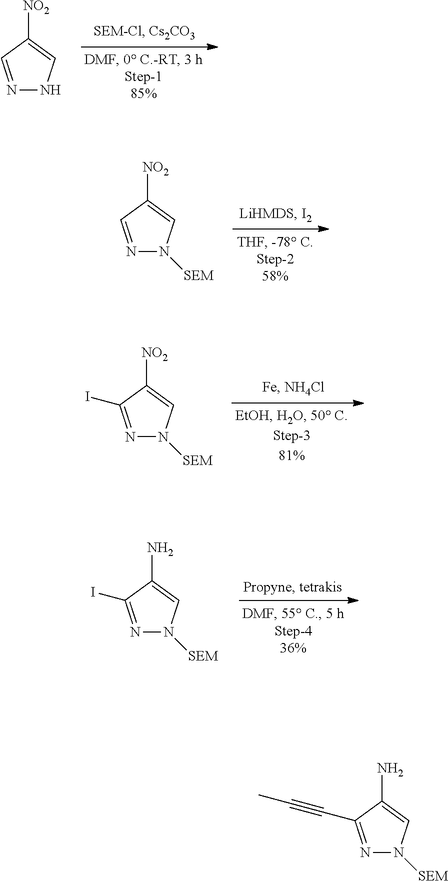 [SMILES: O=[N+]([O-])c1cn[nH]c1 ; C[Si](C)(C)CCOCn1cc([N+](=O)[O-])cn1 ; C[Si](C)(C)CCOCn1cc([N+](=O)[O-])c(I)n1 ; C[Si](C)(C)CCOCn1cc(N)c(I)n1 ; CC#Cc1nn(COCC[Si](C)(C)C)cc1N]

4-nitro-1-((2-(trimethylsilyl)ethoxy)methyl)-1H-pyrazole: To a stirred solution of 4-nitro-1H-pyrazole (5 g, 44.21 mmol) and Cs₂CO₃ (28.7 g, 88.49 mmol) in DMF (50 mL), was added SEM-Cl (11.5 mL, 66.37 mmol) at 0° C. and stirred at room temperature for 3 h. The progress of the reaction was monitored by TLC. After completion of the reaction, the reaction was quenched with ice-cold water and extracted with ethyl acetate. The organic layer was washed with ice-cold water, dried over Na₂SO₄ and concentrated under reduced pressure to give the crude product. The crude compound was purified by Sepa-Bean using silica gel (230-400 mesh), eluting with 0-10% ethyl acetate in pet-ether to give 4-nitro-1-((2-(trimethylsilyl)ethoxy)methyl)-1H-pyrazole as colorless liquid (8 g, 85% yield). ¹H NMR (400 MHz, CDCl₃) δ=8.31 (s, 1H), 8.08-8.14 (m, 1H), 5.45 (s, 2H), 3.59-3.69 (m, 2H), 0.90-1.0 (m, 2H), 0.00-0.05 (m, 9H).

3-iodo-4-nitro-1-((2-(trimethylsilyl)ethoxy)methyl)-1H-pyrazole: To a stirred solution of 4-nitro-1-((2-(trimethylsilyl)ethoxy)methyl)-1H-pyrazole (10 g, 46.94 mmol) in dry THE (100 mL), was added LiHMDS (56 mL) at −78° C. and stirred for 1 h at same temperature. After 1 h, Iodine (10 g, 78.85 mmol) was added at −78° C. and stirred for 1 h. Progress of the reaction was monitored by TLC. After completion of the reaction, the reaction mass was quenched with sat. NH₄Cl, extracted with ethyl acetate. The organic layer was dried over Na₂SO₄, concentrated under reduced pressure to give the crude product. The crude compound was purified by Sepa-Bean using silica gel (230-400 mesh), eluting with 0-30% ethyl acetate in pet-ether to give 3-iodo-4-nitro-1-((2-(trimethylsilyl)ethoxy)methyl)-1H-pyrazole as pale yellow liquid (10 g, 58% yield). ¹H NMR (400 MHz, CDCl₃) δ=8.26 (s, 1H), 5.61 (s, 2H), 3.62-3.69 (m, 2H), 0.90-0.99 (m, 2H), 0.00-0.05 (m, 9H).

3-iodo-1-((2-(trimethylsilyl)ethoxy)methyl)-1H-pyrazol-4-amine: To a stirred solution of 3-iodo-4-nitro-1-((2-(trimethylsilyl)ethoxy)methyl)-1H-pyrazole (3 g, 8.13 mmol) in ethanol (30 mL) and water (30 mL), were added NH₄Cl (4.30 g, 81.30 mmol) and iron powder (4.71 g, 81.30 mmol) at room temperature and then stirred at 50° C. for 3 h. Progress of the reaction was monitored by TLC. After, completion of the reaction, the reaction mass was filtered through plug of celite; the filtrate was concentrated, diluted with ethyl acetate and washed with water. The organic layer was dried over Na₂SO₄, concentrated under reduced pressure to give the crude product. The crude compound was purified by Sepa-Bean using silica gel (230-400 mesh), eluting with 0-30% ethyl acetate in pet-ether to give 3-iodo-1-((2-(trimethylsilyl)ethoxy)methyl)-1H-pyrazol-4-amine as pale brown liquid (2.5 g, 91% yield). ¹H NMR (400 MHz, CDCl₃) δ=7.15-7.28 (m, 1H), 5.31-5.40 (m, 2H), 3.49-3.59 (m, 2H), 2.80 (br-s, 2H), 0.88-0.92 (m, 2H), −0.03-0.02 (m, 9H).

3-(prop-1-yn-1-yl)-1-((2-(trimethylsilyl)ethoxy)methyl)-1H-pyrazol-4-amine: To a degassed stirring solution of 3-iodo-1-((2-(trimethylsilyl)ethoxy)methyl)-1H-pyrazol-4-amine (2 g, 5.89 mmol) in DMF (40 mL), were added CuI (0.22 g, 1.17 mmol), triethyl amine (16 mL, 18V) and tetrakis (0.6 g, 0.58 mmol) and degassed for 10 min. After, propyne (15.7 mL, 11.79 mmol, 3% in heptane) was added and heated at 55° C. for 5 h. Progress of the reaction was monitored by TLC & LCMS. After completion of the reaction, the reaction mass was filtered through celite plug, washed with ethyl acetate. The ethyl acetate layer was washed with ice-cold water. The organic layer was dried over Na₂SO₄, concentrated under reduced pressure to give the crude product. The crude product was purified by Sepa-Bean using silica gel (230-400 mesh), eluting with 0-40% ethyl acetate in pet-ether to give 3-(prop-1-yn-1-yl)-1-((2-(trimethylsilyl)ethoxy)methyl)-1H-pyrazol-4-amine as brown gummy liquid (0.5 g, 36% yield). LC-MS m/z [M+H]+=252.13, ¹H NMR (400 MHz, CDCl₃) δ=7.19 (s, 1H), 5.39 (s, 2H), 3.58-3.64 (m, 2H), 3.05-3.25 (br-s, 2H), 3.17 (s, 3H), 0.90-0.96 (m, 2H), −0.08-0.02 (m, 9H).

As per the method described in Example 2, coupling to Intermediate 1 and deprotection of SEM provided for

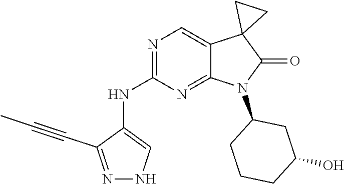 [SMILES: CC#Cc1n[nH]cc1Nc1ncc2c(n1)N([C@@H]1CCC[C@@H](O)C1)C(=O)C21CC1]

7′-((1R,3R)-3-hydroxycyclohexyl)-2′-((3-(prop-1-yn-1-yl)-1H-pyrazol-4-yl)amino)spiro[cyclopropane-1,5′-pyrrolo[2,3-d]pyrimidin]-6′(7′H)-one

### Example 59

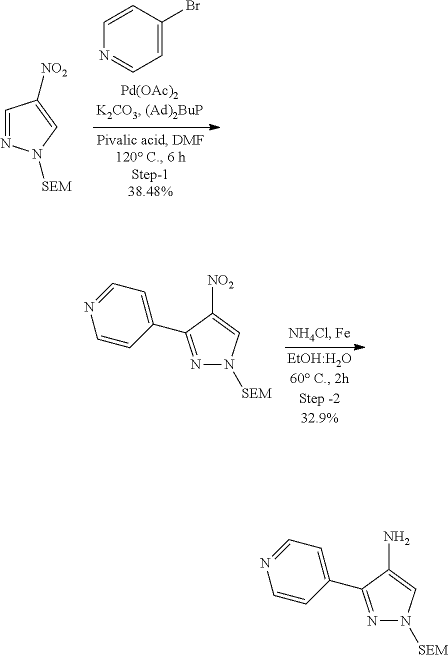 [SMILES: C[Si](C)(C)CCOCn1cc([N+](=O)[O-])cn1 ; Brc1ccncc1 ; C[Si](C)(C)CCOCn1cc([N+](=O)[O-])c(-c2ccncc2)n1 ; C[Si](C)(C)CCOCn1cc(N)c(-c2ccncc2)n1]

4-(4-nitro-1-((2-(trimethylsilyl)ethoxy)methyl)-1H-pyrazol-3-yl)pyridine: To a stirred solution of 4-nitro-1-((2-(trimethylsilyl)ethoxy)methyl)-1H-pyrazole (500 mg, 1.92 mmol) and 4-bromopyridine (592.9 mg, 2.89 mmol) in DMF (5 mL), was added K₂CO₃ (799.3 mg 5.78 mmol), Bis (1-adamantyl-butyl-phosphane (69.13 mg, 0.19 mmol) and Pivalic acid (98.45 mg, 0.96 mmol)) at 25° C. and stirred at 120° C. for 6 h. The progress of the reaction was monitored by TLC. After completion of the reaction, the reaction mass was diluted with water and extracted with ethyl acetate. The organic layer was washed with ice-cold water, dried over Na₂SO₄ and concentrated under reduced pressure to give the crude product. The crude compound was purified by Sepa-Bean using silica gel (230-400 mesh), eluting with 0-40% ethyl acetate in pet-ether to give 4-(4-nitro-1-((2-(trimethylsilyl)ethoxy)methyl)-1H-pyrazol-3-yl)pyridine as pale yellow liquid (300 mg, 38.48% yield). LC-MS m/z [M+H]⁺=321.43

3-(pyridin-4-yl)-1-((2-(trimethylsilyl)ethoxy)methyl)-1H-pyrazol-4-amine: To a stirred solution of 4-(4-nitro-1-((2-(trimethylsilyl)ethoxy)methyl)-1H-pyrazol-3-yl)pyridine (300 mg, 0.93 mmol) in ethanol (3 mL) and water (3 mL), were added NH₄Cl (500.8 mg, 9.36 mmol) and iron powder (522.9 mg, 9.363 mmol) at room temperature and then stirred at 60° C. for 2 h. Progress of the reaction was monitored by TLC. After, completion of the reaction, the reaction mass was filtered through plug of celite; the filtrate was concentrated, diluted with ethyl acetate and washed with water. The organic layer was dried over Na₂SO₄, concentrated under reduced pressure to give the crude product of 3-(pyridin-4-yl)-1-((2-(trimethylsilyl)ethoxy)methyl)-1H-pyrazol-4-amine (200 mg, 32.9% yield) as brown liquid LC-MS m/z [M+H]⁺=291.39.

As per the method described in Example 2, coupling to Intermediate 1 and deprotection of SEM provided for

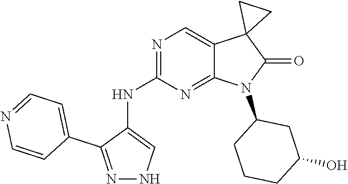 [SMILES: O=C1N([C@@H]2CCC[C@@H](O)C2)c2nc(Nc3c[nH]nc3-c3ccncc3)ncc2C12CC2]

7′-((1R,3R)-3-hydroxycyclohexyl)-2′-((3-(pyridin-4-yl)-1H-pyrazol-4-yl)amino)spiro[cyclopropane-1,5′-pyrrolo[2,3-d]pyrimidin]-6′(7′H)-one

### Example 60

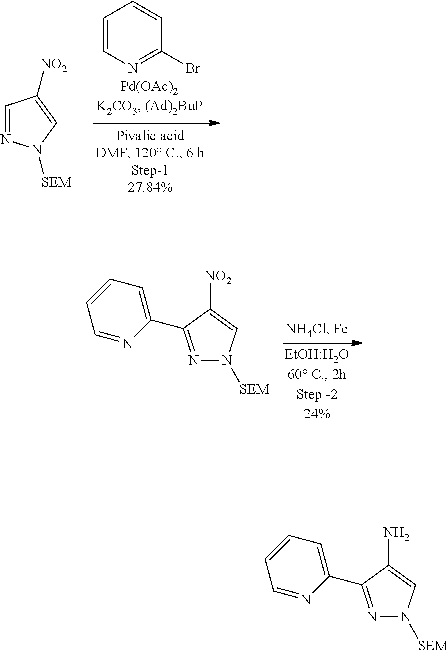 [SMILES: C[Si](C)(C)CCOCn1cc([N+](=O)[O-])cn1 ; Brc1ccccn1 ; C[Si](C)(C)CCOCn1cc([N+](=O)[O-])c(-c2ccccn2)n1 ; C[Si](C)(C)CCOCn1cc(N)c(-c2ccccn2)n1]

2-(4-nitro-1-((2-(trimethylsilyl)ethoxy)methyl)-1H-pyrazol-3-yl)pyridine: To a stirred solution of 4-nitro-1-((2-(trimethylsilyl)ethoxy)methyl)-1H-pyrazole (500 mg, 2.05 mmol) and 2-bromopyridine (487.0 mg, 3.08 mmol) in DMF (5 mL), was added K₂CO₃ (851.9 mg 6.16 mmol), Bis (1-adamantyl-butyl-phosphane (73.67 mg, 0.20 mmol) and Pivalic acid (104.9 mg 0.12 mmol) at 25° C. and stirred at 120° C. for 6 h. The progress of the reaction was monitored by TLC. After completion of the reaction, the reaction was diluted with water and extracted with ethyl acetate. The organic layer was washed with ice-cold water, dried over Na₂SO₄ and concentrated under reduced pressure to give the crude product. The crude compound was purified by Sepa-Bean using silica gel (230-400 mesh), eluting with 20-30% ethyl acetate in pet-ether to give 2-(4-nitro-1-((2-(trimethylsilyl)ethoxy)methyl)-1H-pyrazol-3-yl)pyridine (300 mg, 27.84% yield) as pale yellow liquid. LC-MS m/z [M+H]⁺=321.43

3-(pyridin-2-yl)-1-((2-(trimethylsilyl)ethoxy)methyl)-1H-pyrazol-4-amine: To a stirred solution of 2-(4-nitro-1-((2-(trimethylsilyl)ethoxy)methyl)-1H-pyrazol-3-yl)pyridine (300 mg, 0.93 mmol) in ethanol (3 ml) and water (3 ml), were added NH₄Cl (500.8 mg, 9.363 mmol) and iron powder (522.9 mg, 9.363 mmol) at room temperature and then stirred at 60° C. for 2 h. Progress of the reaction was monitored by TLC. After, completion of the reaction, the reaction mass was filtered through plug of celite; the filtrate was concentrated, diluted with ethyl acetate and washed with water. The organic layer was dried over Na₂SO₄, concentrated under reduced pressure to give the crude product of 3-(pyridin-2-yl)-1-((2-(trimethylsilyl)ethoxy)methyl)-1H-pyrazol-4-amine (200 mg, 24% yield) as brown liquid. LC-MS m/z [M+H]⁺=291.43.

As per the method described in Example 2, coupling to Intermediate 1 and deprotection of SEM provided for

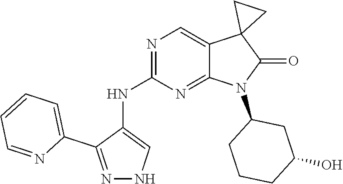 [SMILES: O=C1N([C@@H]2CCC[C@@H](O)C2)c2nc(Nc3c[nH]nc3-c3ccccn3)ncc2C12CC2]

7′-((1R,3R)-3-hydroxycyclohexyl)-2′-((3-(pyridin-2-yl)-1H-pyrazol-4-yl)amino)spiro[cyclopropane-1,5′-pyrrolo[2,3-d]pyrimidin]-6′(7′H)-one

### Example 61

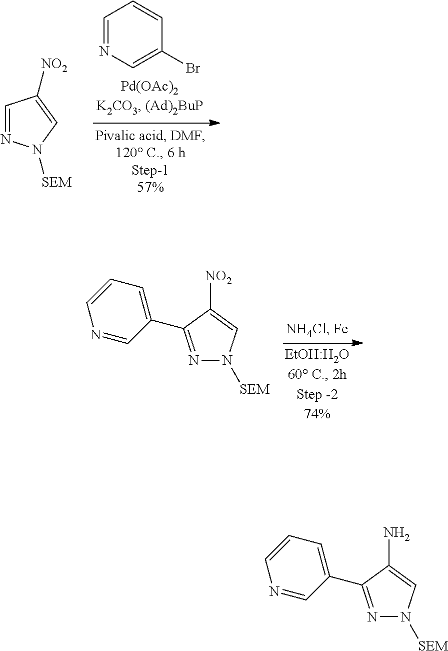 [SMILES: C[Si](C)(C)CCOCn1cc([N+](=O)[O-])cn1 ; Brc1cccnc1 ; C[Si](C)(C)CCOCn1cc([N+](=O)[O-])c(-c2cccnc2)n1 ; C[Si](C)(C)CCOCn1cc(N)c(-c2cccnc2)n1]

3-(4-nitro-1-((2-(trimethylsilyl)ethoxy)methyl)-1H-pyrazol-3-yl)pyridine: To a stirred solution of 4-nitro-1-((2-(trimethylsilyl)ethoxy)methyl)-1H-pyrazole (500 mg, 2.055 mmol) and 3-bromopyridine (487.0 mg, 3.082 mmol) in DMF (5 mL), was added K₂CO₃ (851.9 mg 6.164 mmol), Bis (1-adamantyl-butyl-phosphane (73.67 mg, 0.20 mmol) and Pivalic acid 104.9 mg 0.102 mmol)) at 25° C. and stirred at 120° C. for 6 h. The progress of the reaction was monitored by TLC. After completion of the reaction, the reaction was diluted with water and extracted with ethyl acetate. The organic layer was washed with ice-cold water, dried over Na₂SO₄ and concentrated under reduced pressure to give the crude product. The crude compound was purified by Sepa-Bean using silica gel (230-400 mesh), eluting with 20-30% ethyl acetate in pet-ether to give 3-(4-nitro-1-((2-(trimethylsilyl)ethoxy)methyl)-1H-pyrazol-3-yl)pyridine (400 mg, 57.77% yield) as pale yellow liquid. LC-MS m/z [M+H]+=321.34.

3-(pyridin-3-yl)-1-((2-(trimethylsilyl)ethoxy)methyl)-1H-pyrazol-4-amine: To a stirred solution of 3-(4-nitro-1-((2-(trimethylsilyl)ethoxy)methyl)-1H-pyrazol-3-yl)pyridine (400 mg, 1.248 mmol) in ethanol (4 ml) and water (4 ml), were added NH₄Cl (667.7 mg, 12.48 mmol) and iron powder (697.2 mg, 12.48 mmol) at room temperature and then stirred at 60° C. for 2 h. Progress of the reaction was monitored by TLC. After, completion of the reaction, the reaction mass was filtered through plug of celite; the filtrate was concentrated, diluted with ethyl acetate and washed with water. The organic layer was dried over Na₂SO₄, concentrated under reduced pressure to give the crude product of 3-(pyridin-3-yl)-1-((2-(trimethylsilyl)ethoxy)methyl)-1H-pyrazol-4-amine (300 mg, 74.78% yield) as brown liquid. LC-MS m/z [M+H]+=291.43.

As per the method described in Example 2, coupling to Intermediate 1 and deprotection of SEM provided for

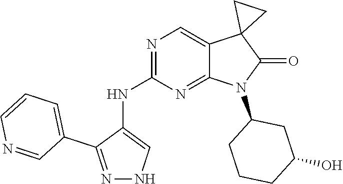 [SMILES: O=C1N([C@@H]2CCC[C@@H](O)C2)c2nc(Nc3c[nH]nc3-c3cccnc3)ncc2C12CC2]

7′-((1R,3R)-3-hydroxycyclohexyl)-2′-((3-(pyridin-3-yl)-1H-pyrazol-4-yl)amino)spiro[cyclopropane-1,5′-pyrrolo[2,3-d]pyrimidin]-6′(7′H)-one

### Example 62

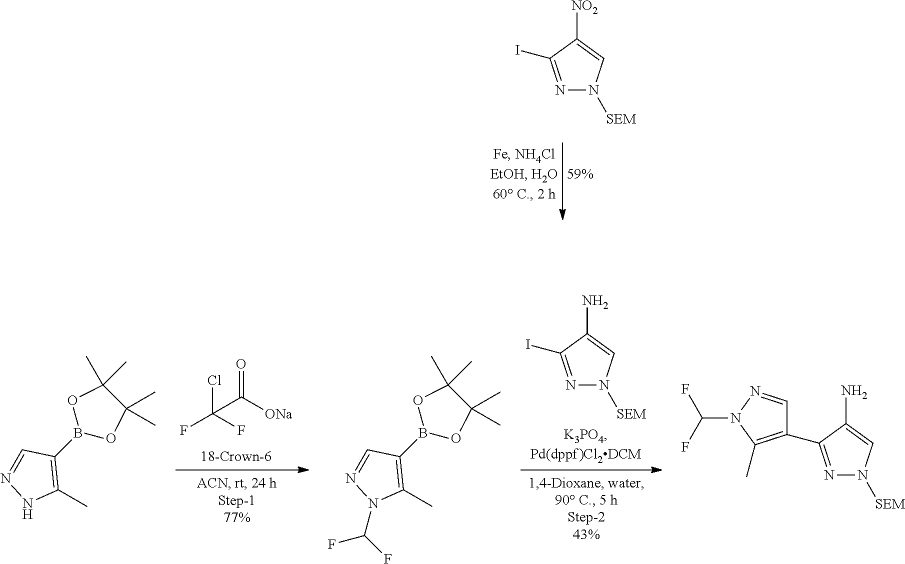

3-iodo-1-((2-(trimethylsilyl)ethoxy)methyl)-1H-pyrazol-4-amine: To a stirred solution of 3-iodo-4-nitro-1-((2-(trimethylsilyl)ethoxy)methyl)-1H-pyrazole (5 g, 13.54 mmol) in ethanol (50 ml) and water (50 ml), were added NH₄Cl (7.243 g, 135.4 mmol) and iron powder (7.563 g, 135.4 mmol) and stirred at 60° C. for 2 h. Progress of the reaction was monitored by TLC. After, completion of the reaction, the reaction mass was filtered through plug of celite; the filtrate was concentrated, diluted with ethyl acetate and washed with water. The organic layer was dried over Na₂SO₄, concentrated under reduced pressure to give the crude product of 3-iodo-1-((2-(trimethylsilyl)ethoxy)methyl)-1H-pyrazol-4-amine (3.0 g, 59% yield) as brown liquid. LC-MS m/z [M+H]+=340.30.

1-(difluoromethyl)-5-methyl-4-(4,4,5,5-tetramethyl-1,3,2-dioxaborolan-2-yl)-1H-pyrazole: To a stirred solution of 5-methyl-4-(4,4,5,5-tetramethyl-1,3,2-dioxaborolan-2-yl)-1H-pyrazole (10.0 g, 48.06 mmol) and sodium 2-chloro-2,2-difluoroacetate (8.9 g, 58.38 mmol) in ACN (100 mL), was added 18-Crown-6 (2.540 g 9.61 mmol), and stirred at room temperature for 24 h. Progress of the reaction was monitored by TLC/LCMS. After completion of the reaction, the reaction mixture was concentrated under reduced pressure to give the crude product. The crude compound was purified by Sepa-Bean using silica gel (230-400 mesh), eluting with 5-10% ethyl acetate in pet-ether to give 1-(difluoromethyl)-5-methyl-4-(4,4,5,5-tetramethyl-1,3,2-dioxaborolan-2-yl)-1H-pyrazole (7.0 g, 77% yield) as colour less liquid. LC-MS m/z [M+H]+=259.32,

1′-(difluoromethyl)-5′-methyl-1-((2-(trimethylsilyl)ethoxy)methyl)-1H,1′H-[3,4′-bipyrazol]-4-amine: To a degassed solution of 1-(difluoromethyl)-5-methyl-4-(4,4,5,5-tetramethyl-1,3,2-dioxaborolan-2-yl)-1H-pyrazole (1 g, 2.94 mmol), 3-iodo-1-((2-(trimethylsilyl)ethoxy)methyl)-1H-pyrazol-4-amine (912.9 mg, 3.53 mmol) and K₃PO₄ (1.567 g 7.36 mmol) in 1,4-Dioxane (9 mL) and Water (1 mL) was added Pd(dppf)Cl₂·DCM (240.7 mg, 0.29 mmol) stirred at 90° C. for 5 h. Progress of the reaction was monitored by TLC and LCMS. After completion of the reaction, the reaction mass was filtered, diluted with ethyl acetate (20 mL) and washed with water (2×30 mL). The organic layer was dried over Na₂SO₄, concentrated under reduced pressure to give the crude product. The crude compound was purified by Sepa-Bean using silica gel (230-400 mesh), eluting with 30-40% EtOAc in pet ether to give 1′-(difluoromethyl)-5′-methyl-1-((2-(trimethylsilyl)ethoxy)methyl)-1H,1′H-[3,4′-bipyrazol]-4-amine (500 mg, 43.03% yield) as brown colour gummy solid. LC-MS m/z [M+H]+=344.15.

As per the method described in Example 2, coupling to Intermediate 1 and deprotection of SEM provided for

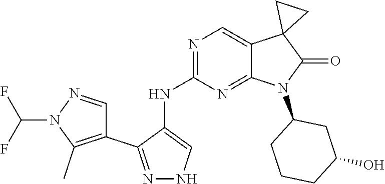 [SMILES: Cc1c(-c2n[nH]cc2Nc2ncc3c(n2)N([C@@H]2CCC[C@@H](O)C2)C(=O)C32CC2)cnn1C(F)F]

2-((1′-(difluoromethyl)-5-methyl-1H,1′H-[3,4′-bipyrazol]-4-yl)amino)-7′-((1R,3R)-3-hydroxycyclohexyl)spiro[cyclopropane-1,5′-pyrrolo[2,3-d]pyrimidin]-6′(7′H)-one

### Example 63

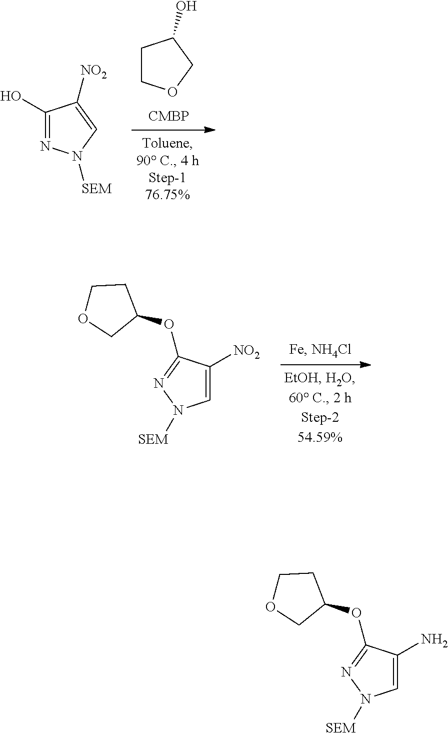 [SMILES: C[Si](C)(C)CCOCn1cc([N+](=O)[O-])c(O)n1 ; O[C@H]1CCOC1 ; C[Si](C)(C)CCOCn1cc([N+](=O)[O-])c(O[C@@H]2CCOC2)n1 ; C[Si](C)(C)CCOCn1cc(N)c(O[C@@H]2CCOC2)n1]

(R)-4-nitro-3-((tetrahydrofuran-3-yl)oxy)-1-((2-(trimethylsilyl)ethoxy)methyl)-1H-pyrazole: To a stirred solution of 4-nitro-1-((2-(trimethylsilyl)ethoxy)methyl)-1H-pyrazol-3-ol (1.0 g, 3.85 mmol) and (S)-tetrahydrofuran-3-ol (509.6 mg, 5.78 mmol) in toluene (30 mL), was added CMBP (1.861 g 7.71 mmol) and stirred at 90° C. for 4 h. The progress of the reaction was monitored by TLC. After completion of the reaction, the reaction mixture concentrated under reduced pressure to give the crude product. The crude compound was purified by Sepa-Bean using silica gel (230-400 mesh), eluting with 20-30% ethyl acetate in pet-ether to give (R)-4-nitro-3-((tetrahydrofuran-3-yl)oxy)-1-((2-(trimethylsilyl)ethoxy)methyl)-1H-pyrazole (1.0 g, 76.75% yield) as pale yellow liquid. LC-MS m/z [M+H]⁺=330.49.

(R)-3-((tetrahydrofuran-3-yl)oxy)-1-((2-(trimethylsilyl)ethoxy)methyl)-1H-pyrazol-4-amine: To a stirred solution of R)-4-nitro-3-((tetrahydrofuran-3-yl)oxy)-1-((2-(trimethylsilyl)ethoxy)methyl)-1H-pyrazole (1.0 g, 3.03 mmol) in ethanol (10 ml) and water (10 ml), were added NH₄Cl (1.62 g, 30.36 mmol) and iron powder (1.69 mg, 30.36 mmol) and stirred at 60° C. for 2 h. Progress of the reaction was monitored by TLC. After, completion of the reaction, the reaction mass was filtered through plug of celite; the filtrate was concentrated, diluted with ethyl acetate and washed with water. The organic layer was dried over Na₂SO₄, concentrated under reduced pressure to give the crude product of (R)-3-((tetrahydrofuran-3-yl)oxy)-1-((2-(trimethylsilyl)ethoxy)methyl)-1H-pyrazol-4-amine (900 mg, 54.59% yield) as brown liquid. LC-MS m/z [M+H]⁺=300.50

As per the method described in Example 2, coupling to Intermediate 1 and deprotection of SEM provided for

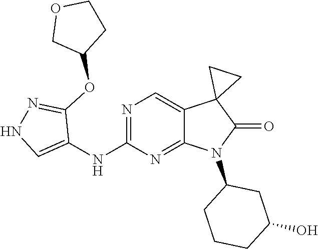

OH 7′-((1R,3R)-3-hydroxycyclohexyl)-2′-((3-(((R)-tetrahydrofuran-3-yl)oxy)-1H-pyrazol-4-yl)amino)spiro[cyclopropane-1,5′-pyrrolo[2,3-d]pyrimidin]-6′(7′H)-one

### Example 64

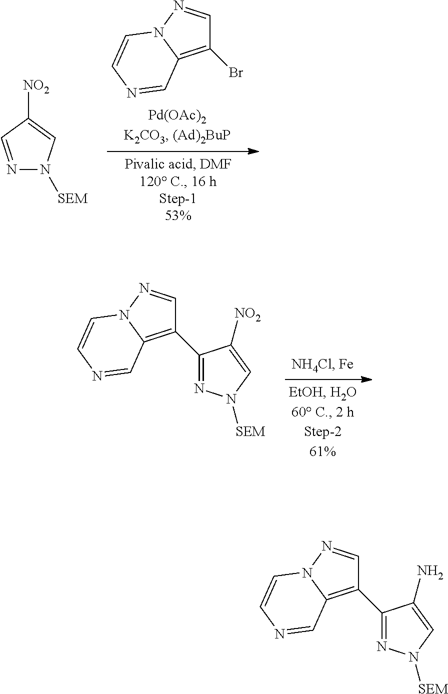 [SMILES: C[Si](C)(C)CCOCn1cc([N+](=O)[O-])cn1 ; Brc1cnn2ccncc12 ; C[Si](C)(C)CCOCn1cc([N+](=O)[O-])c(-c2cnn3ccncc23)n1 ; C[Si](C)(C)CCOCn1cc(N)c(-c2cnn3ccncc23)n1]

3-(4-nitro-1-((2-(trimethylsilyl)ethoxy)methyl)-1H-pyrazol-3-yl)pyrazolo[1,5-a]pyrazine: To a stirred solution of 4-nitro-1-((2-(trimethylsilyl)ethoxy)methyl)-1H-pyrazole (400 mg, 1.64 mmol) and 3-bromopyrazolo[1,5-a]pyrazine (488.3 mg, 2.46 mmol) in DMF (4 mL), was added K₂CO₃ (681.5 mg 4.93 mmol), Bis (1-adamantyl-butyl-phosphane (58.94 mg, 0.16 mmol) and Pivalic acid (83.94 mg 0.88 mmol) at 25° C. and stirred at 120° C. for 16 h. TLC monitored the progress of the reaction. After completion of the reaction, the reaction was diluted with water and extracted with ethyl acetate. The organic layer was washed with ice-cold water, dried over Na₂SO₄ and concentrated under reduced pressure to give the crude product. The crude compound was purified by Sepa-Bean using silica gel (230-400 mesh), eluting with 30-40% ethyl acetate in pet-ether to give 3-(4-nitro-1-((2-(trimethylsilyl)ethoxy)methyl)-1H-pyrazol-3-yl)pyrazolo[1,5-a]pyrazine (400 mg, 53.08% yield) as pale yellow liquid. LC-MS m/z [M+H]⁺=361.54

3-(pyrazolo[1,5-a]pyrazin-3-yl)-1-((2-(trimethylsilyl)ethoxy)methyl)-1H-pyrazol-4-amine: To a stirred solution of 3-(4-nitro-1-((2-(trimethylsilyl)ethoxy)methyl)-1H-pyrazol-3-yl)pyrazolo[1,5-a]pyrazine (400 mg, 1.11 mmol) in ethanol (4 ml) and water (4 ml), were added NH₄Cl (593.6 mg, 11.10 mmol) and iron powder (619.8 mg, 11.10 mmol) at room temperature and then stirred at 60° C. for 2 h. Progress of the reaction was monitored by TLC. After, completion of the reaction, the reaction mass was filtered through plug of celite; the filtrate was concentrated, diluted with ethyl acetate (20 mL) and washed with water (2×30 mL). The organic layer was dried over Na₂SO₄, concentrated under reduced pressure to give the crude product of 3-(pyrazolo[1,5-a]pyrazin-3-yl)-1-((2-(trimethylsilyl)ethoxy)methyl)-1H-pyrazol-4-amine (300 mg, 61.46% yield) as brown liquid. LC-MS m/z [M+H]⁺=331.51

As per the method described in Example 2, coupling to Intermediate 1 and deprotection of SEM provided for

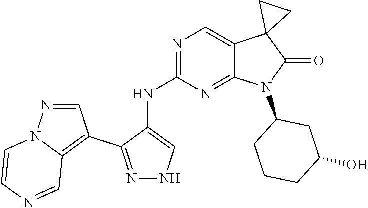 [SMILES: O=C1N([C@@H]2CCC[C@@H](O)C2)c2nc(Nc3c[nH]nc3-c3cnn4ccncc34)ncc2C12CC2]

7′-((1R,3R)-3-hydroxycyclohexyl)-2′-((3-(pyrazolo[1,5-a]pyrazin-3-yl)-1H-pyrazol-4-yl)amino)spiro[cyclopropane-1,5′-pyrrolo[2,3-d]pyrimidin]-6′(7′H)-one

### Example 65

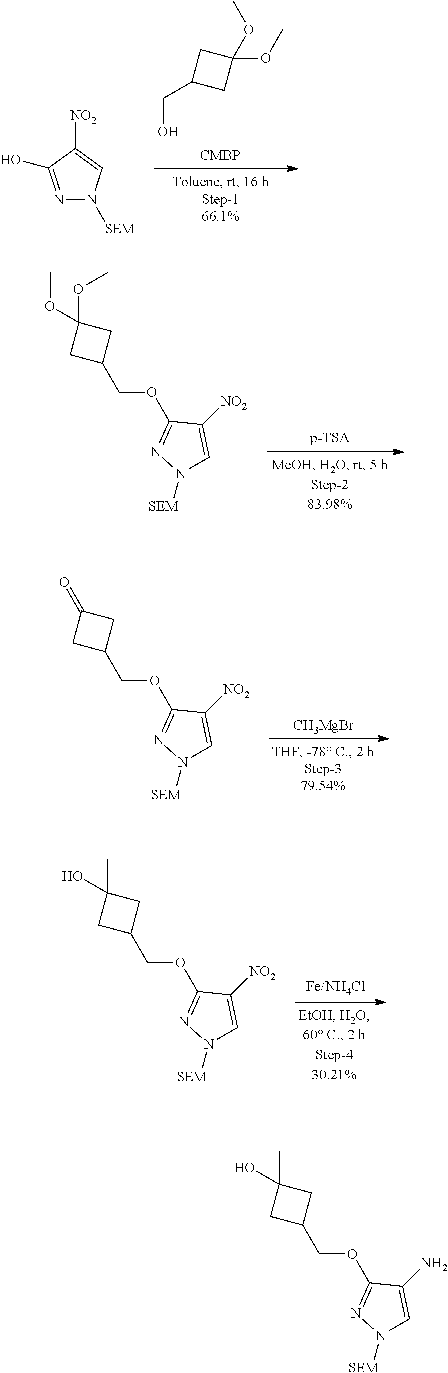

3-((3,3-dimethoxycyclobutyl)methoxy)-4-nitro-1-((2-(trimethylsilyl)ethoxy)methyl)-1H-pyrazole: To a stirred solution of 4-nitro-1-((2-(trimethylsilyl)ethoxy)methyl)-1H-pyrazol-3-ol (500 mg, 1.92 mmol) and (3,3-dimethoxycyclobutyl)methanol (563.7 mg, 3.85 mmol) in toluene (15 mL), was added Cyanomethylene)tributylphosphorane (698.0 mg 2.89 mmol) and stirred at room temperature for 16 h. The progress of the reaction was monitored by TLC. After completion of the reaction, the reaction mixture concentrated under reduced pressure to give the crude product. The crude compound was purified by Sepa-Bean using silica gel (230-400 mesh), eluting with 20-30% ethyl acetate in pet-ether to give 3-((3,3-dimethoxycyclobutyl)methoxy)-4-nitro-1-((2-(trimethylsilyl)ethoxy)methyl)-1H-pyrazole (510 mg, 66.11% yield) as brown gummy liquid. ¹H NMR (400 MHz, DMSO-d₆) δ=8.139 (s, 1H), 5.246 (s, 2H), 4.347 (d, J=7.2 Hz, 2H), 3.63 (t, J=8.0 Hz 2H), 3.1575 (d, J=6.8 Hz, 6H), 2.40-2.345 (m, 1H), 2.029-2.012 (m, 2H), 2.004-1.996 (m, 2H), 0.949-0.9281 (m, 2H), 0.101 (s, 9H).

3-(((4-nitro-1-((2-(trimethylsilyl)ethoxy)methyl)-1H-pyrazol-3-yl)oxy)methyl)cyclobutan-1-one: To a stirred solution of 3-((3,3-dimethoxycyclobutyl)methoxy)-4-nitro-1-((2-(trimethylsilyl)ethoxy)methyl)-1H-pyrazole (500 mg, 1.29 mmol) in methanol (2.5 mL) and water (1.5 mL) was added p-TSA (1.22 g 6.45 mmol), at 0° C. and stirred at room temperature for 5 h. The progress of the reaction was monitored by TLC. After completion of the reaction, the reaction was diluted with water and extracted with ethyl acetate. The organic layer was washed with ice-cold water, dried over Na₂SO₄ and concentrated under reduced pressure to give the crude product. The crude compound was purified by Sepa-Bean using silica gel (230-400 mesh), eluting with 10-20% ethyl acetate in pet-ether to give 3-(((4-nitro-1-((2-(trimethylsilyl)ethoxy)methyl)-1H-pyrazol-3-yl)oxy)methyl)cyclobutan-1-one (370 mg, 83.98% yield) as pale yellow liquid. ¹H NMR (400 MHz, DMSO-d₆) δ=8.161 (s, 1H), 5.258 (s, 2H), 4.475 (d, J=2.8 Hz, 2H), 3.656-3.614 (m, 2H), 3.288-3.207 (m, 2H), 3.071-3.001 (m, 3H), 0.912-1.012 (m, 2H) 0.101 (s, 9H).

1-methyl-3-(((4-nitro-1-((2-(trimethylsilyl)ethoxy)methyl)-1H-pyrazol-3-yl)oxy)methyl)cyclobutan-1-ol: To a stirred solution of 3-(((4-nitro-1-((2-(trimethylsilyl)ethoxy)methyl)-1H-pyrazol-3-yl)oxy)methyl)cyclobutan-1-one (360 mg, 1.05 mmol) in THE (5.4 ml) was added CH₃MgBr (593.6 mg, 11.10 mmol) at −78° C. and then stirred at same temperature 2 h. Progress of the reaction was monitored by TLC. After, completion of the reaction, the reaction mass was quenched with NH₄Cl solution & extracted with ethyl acetate. The organic layer was dried over Na₂SO₄, concentrated under reduced pressure to give the crude product of 1-methyl-3-(((4-nitro-1-((2-(trimethylsilyl)ethoxy)methyl)-1H-pyrazol-3-yl)oxy)methyl)cyclobutan-1-ol (300 mg, 79.59% yield) as brown liquid.

3-(((4-amino-1-((2-(trimethylsilyl)ethoxy)methyl)-1H-pyrazol-3-yl)oxy)methyl)-1-methylcyclobutan-1-ol: To a stirred solution of 1-methyl-3-(((4-nitro-1-((2-(trimethylsilyl)ethoxy)methyl)-1H-pyrazol-3-yl)oxy)methyl)cyclobutan-1-ol (300 mg, 0.83 mmol) in ethanol (3.5 ml) and water (3.5 ml), were added NH₄Cl (448.9 mg, 8.392 mmol) and iron powder (468.7 mg, 8.392 mmol) and stirred at 60° C. for 2 h. Progress of the reaction was monitored by TLC. After completion of the reaction, the reaction mass was filtered through plug of celite, the filtrate was concentrated, diluted with ethyl acetate and washed with water. The organic layer was dried over Na₂SO₄, concentrated under reduced pressure to give the crude product of 3-(((4-amino-1-((2-(trimethylsilyl)ethoxy)methyl)-1H-pyrazol-3-yl)oxy)methyl)-1-methylcyclobutan-1-ol (200 mg, 30.21% yield) as brown liquid. LC-MS m/z [M+H]+=328.19

As per the method described in Example 2, coupling to Intermediate 1 and deprotection of SEM provided for

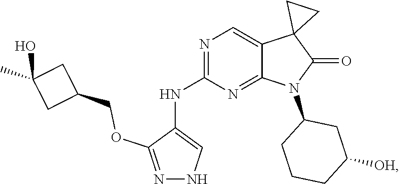 [SMILES: C[C@]1(O)C[C@@H](COc2n[nH]cc2Nc2ncc3c(n2)N([C@@H]2CCC[C@@H](O)C2)C(=O)C32CC2)C1]

2′-((3-(((1r,3R)-3-hydroxy-3-methylcyclobutyl)methoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

### Example 66

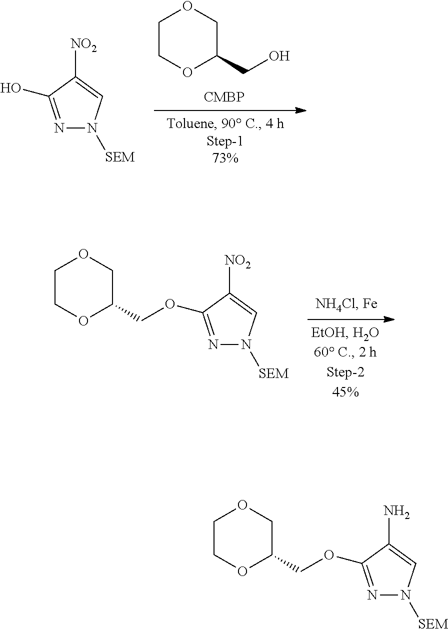 [SMILES: C[Si](C)(C)CCOCn1cc([N+](=O)[O-])c(O)n1 ; OC[C@@H]1COCCO1 ; C[Si](C)(C)CCOCn1cc([N+](=O)[O-])c(OC[C@H]2COCCO2)n1 ; C[Si](C)(C)CCOCn1cc(N)c(OC[C@H]2COCCO2)n1]

(R)-3-((1,4-dioxan-2-yl)methoxy)-4-nitro-1-((2-(trimethylsilyl)ethoxy)methyl)-1H-pyrazole: To a stirred solution of 4-nitro-1-((2-(trimethylsilyl)ethoxy)methyl)-1H-pyrazol-3-ol (500 mg, 1.92 mmol) and (R)-(1,4-dioxan-2-yl)methanol (341.6 mg, 2.89 mmol) in Toluene (15.0 mL), was added (Cyanomethylene)tributylphosphorane (930.7 mg 3.85 mmol) C and stirred at 90° C. for 4 h. The progress of the reaction was monitored by TLC. After completion of the reaction, the reaction was concentrated under reduced pressure to give the crude product. The crude compound was purified by Sepa-Bean using silica gel (230-400 mesh), eluting with 20-30% ethyl acetate in pet-ether to give (R)-3-((1,4-dioxan-2-yl)methoxy)-4-nitro-1-((2-(trimethylsilyl)ethoxy)methyl)-1H-pyrazole (600 mg, 73.69% yield) as pale yellow liquid. LC-MS m/z [M+H]⁺=360.23

(R)-3-((1,4-dioxan-2-yl)methoxy)-1-((2-(trimethylsilyl)ethoxy)methyl)-1H-pyrazol-4-amine: To a stirred solution of (R)-3-((1,4-dioxan-2-yl)methoxy)-4-nitro-1-((2-(trimethylsilyl)ethoxy)methyl)-1H-pyrazole (600 mg, 1.66 mmol) in ethanol (6 ml) and water (6 ml), were added NH₄Cl (892.9 mg, 16.69 mmol) and iron powder (932.3 mg, 16.69 mmol) and stirred at 60° C. for 2 h. Progress of the reaction was monitored by TLC. After completion of the reaction, the reaction mass was filtered through plug of celite; the filtrate was concentrated, diluted with ethyl acetate and washed with water. The organic layer was dried over Na₂SO₄, concentrated under reduced pressure to give the crude product of (R)-3-((1,4-dioxan-2-yl)methoxy)-1-((2-(trimethylsilyl)ethoxy)methyl)-1H-pyrazol-4-amine (500 mg, 45% yield) as brown liquid. LC-MS m/z [M+H]⁺=330.31.

As per the method described in Example 2, coupling to Intermediate 1 and deprotection of SEM provided for

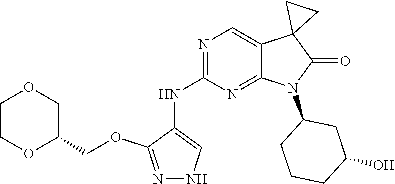 [SMILES: O=C1N([C@@H]2CCC[C@@H](O)C2)c2nc(Nc3c[nH]nc3OC[C@H]3COCCO3)ncc2C12CC2]

2′-((3-(((R)-1,4-dioxan-2-yl)methoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

### Example 67

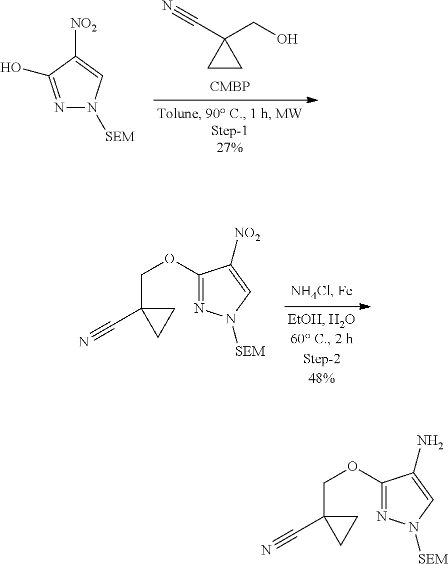 [SMILES: C[Si](C)(C)CCOCn1cc([N+](=O)[O-])c(O)n1 ; N#CC1(CO)CC1 ; C[Si](C)(C)CCOCn1cc([N+](=O)[O-])c(OCC2(C#N)CC2)n1 ; C[Si](C)(C)CCOCn1cc(N)c(OCC2(C#N)CC2)n1]

1-(((4-nitro-1-((2-(trimethylsilyl)ethoxy)methyl)-1H-pyrazol-3-yl)oxy)methyl)cyclopropane-1-carbonitrile: To a stirred solution of 4-nitro-1-((2-(trimethylsilyl)ethoxy)methyl)-1H-pyrazol-3-ol (500 mg, 1.92 mmol) and 1-(hydroxymethyl)cyclopropane-1-carbonitrile (280.9 mg, 2.89 mmol) in toluene (15 mL), was added (Cyanomethylene) tributylphosphorane (930.7 mg 3.85 mmol) and stirred at 60° C. for 1 h. The progress of the reaction was monitored by TLC. After completion of the reaction, the reaction concentrated under reduced pressure to give the crude product. The crude compound was purified by Sepa-Bean using silica gel (230-400 mesh), eluting with 20-30% ethyl acetate in pet-ether to give 1-(((4-nitro-1-((2-(trimethylsilyl)ethoxy)methyl)-1H-pyrazol-3-yl)oxy)methyl)cyclopropane-1-carbonitrile (300 mg, 27.74% yield) as pale yellow liquid. ¹H NMR (400 MHz, CDCl₃) δ=8.150 (s, 1H), 5.241 (s, 2H), 4.348 (s, 2H), 3.634-3.592 (m, 2H), 1.435-1.403 (m, 2H), 1.225-1.193 (m, 2H), 0.958-0.9517 (m, 2H).

1-(((4-amino-1-((2-(trimethylsilyl)ethoxy)methyl)-1H-pyrazol-3-yl)oxy)methyl)cyclopropane-1-carbonitrile: To a stirred solution of 1-(((4-nitro-1-((2-(trimethylsilyl)ethoxy)methyl)-1H-pyrazol-3-yl)oxy)methyl)cyclopropane-1-carbonitrile (300 mg, 0.88 mmol) in ethanol (3 ml) and water (3 ml), were added NH₄Cl (474.1 mg, 8.86 mmol) and iron powder (495.1 mg, 8.864 mmol) and stirred at 60° C. for 2 h.

Progress of the reaction was monitored by TLC. After completion of the reaction, the reaction mass was filtered through plug of celite; the filtrate was concentrated, diluted with ethyl acetate and washed with water. The organic layer was dried over Na₂SO₄, concentrated under reduced pressure to give the crude product of 1-(((4-amino-1-((2-(trimethylsilyl)ethoxy)methyl)-1H-pyrazol-3-yl)oxy)methyl)cyclopropane-1-carbonitrile (200 mg, 48% yield) as brown liquid. LC-MS m/z [M+H]+=309.45.

As per the method described in Example 2, coupling to Intermediate 1 and deprotection of SEM provided for

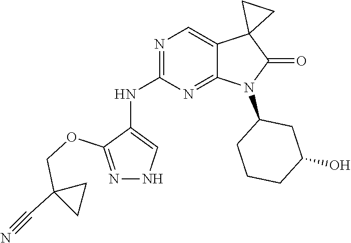 [SMILES: N#CC1(COc2n[nH]cc2Nc2ncc3c(n2)N([C@@H]2CCC[C@@H](O)C2)C(=O)C32CC2)CC1]

1-(((4-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)-1H-pyrazol-3-yl)oxy)methyl)cyclopropane-1-carbonitrile

### Example 68

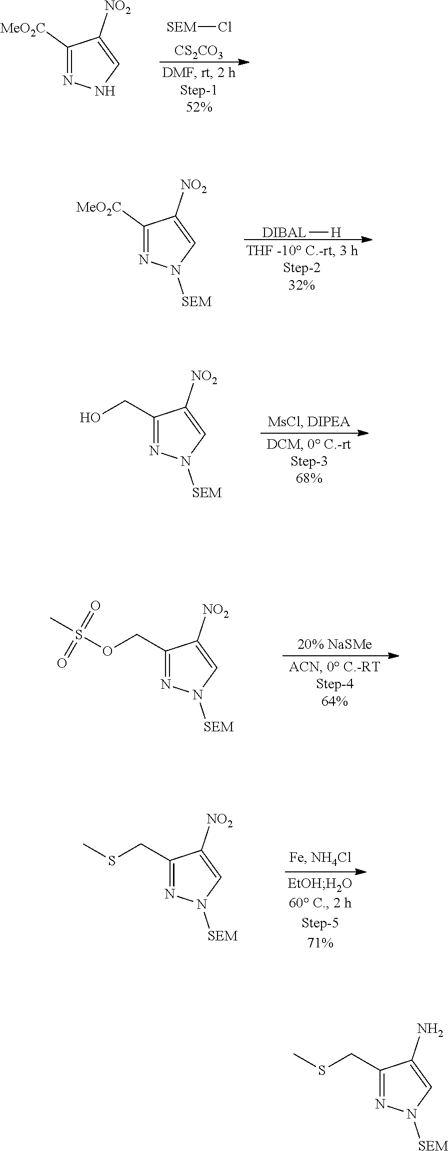

methyl 4-nitro-1-((2-(trimethylsilyl)ethoxy)methyl)-1H-pyrazole-3-carboxylate: To a stirred suspension of methyl 4-nitro-1H-pyrazole-3-carboxylate (15 g, 87.66 mmol) and Cs₂CO₃ (42.7 g, 105.19 mmol) in DMF (150 mL), was added SEM-Cl (22.38 ml, 105.19 mmol) at 0° C. and stirred at 25° C. for 2 h. The progress of the reaction was monitored by TLC. After completion of the reaction, the reaction mass was diluted with, water and extracted with EtOAc. The organic layer dried over Na₂SO₄ and concentrated under reduced pressure to get the crude product. The crude compound was purified by Sepa-Bean using silica gel (230-400 mesh), eluting with 20-30% ethyl acetate in pet-ether to give methyl 4-nitro-1-((2-(trimethylsilyl)ethoxy)methyl)-1H-pyrazole-3-carboxylate (13.56 g, 52% yield) as pale yellow liquid. ¹H NMR (400 MHz, CDCl3, Mixture of regioisomers) δ=8.348 (s, 1H), 8.076 (s, 1H), 5.629 (s, 2H), 5.507 (s, 2H), 4.0175 (d, J=3.8 Hz, 5H), 3.687-3.468 (m, 2H), 3.603-561 (m, 2H), 1.005-0.890 (m, 4H), 0.452-0.19 (m, 12H).

(4-nitro-1-((2-(trimethylsilyl)ethoxy)methyl)-1H-pyrazol-3-yl)methanol: To a stirred solution of methyl 4-nitro-1-((2-(trimethylsilyl)ethoxy)methyl)-1H-pyrazole-3-carboxylate (13.56 g, 44.99 mmol) in THE (135.6 ml), was added DIBAL-H (89.9 mL, 89.989 mmol) at −10° C. and then stirred at 25° C. for 3 h. Progress of the reaction was monitored by TLC. After completion of the reaction, the reaction mass was quenched with aq. NH₄Cl and extracted with ethyl acetate. The organic layer was washed with water, dried over Na₂SO₄, concentrated under reduced pressure to give the crude product. The crude compound was purified by Sepa-Bean using silica gel (230-400 mesh), eluting with 20-30% ethyl acetate in Pet-ether to give (4-nitro-1-((2-(trimethylsilyl)ethoxy)methyl)-1H-pyrazol-3-yl)methanol (4.2 g, 32.7% yield) as Pale red liquid. LC-MS m/z [M+H]+=274.19.

(4-nitro-1-((2-(trimethylsilyl)ethoxy)methyl)-1H-pyrazol-3-yl)methyl methanesulfonate: To a stirred solution of (4-nitro-1-((2-(trimethylsilyl)ethoxy)methyl)-1H-pyrazol-3-yl)methanol (4.2 g, 15.36 mmol) and DIPEA (3.47 mL, 19.96 mmol) in DCM (20 ml) was added Mesyl chloride (1.33 mL, 17.20 mmol) at 0° C. and then stirred at room temperature for 3 h. Progress of the reaction was monitored by TLC. After completion of the reaction, the reaction mass was diluted with ethyl acetate and washed with water. The organic layer was dried over Na₂SO₄, concentrated under reduced pressure to give (4-nitro-1-((2-(trimethylsilyl)ethoxy)methyl)-1H-pyrazol-3-yl)methyl methanesulfonate (3.6 g, 67.92% yield) as brown liquid. The crude product was used as such for next step without further purification. ¹H NMR (400 MHz, DMSO-d₆) δ=8.319 (s, 1H), 7.80-8.04 (m, 2H), 5.424 (s, 1H), 4.875 (s, 1H), 3.673-3.616 (m, 2H), 0.962-0.920 (m, 1H), 0.000 (s, 9H).

3-((methylthio)methyl)-4-nitro-1-((2-(trimethylsilyl)ethoxy)methyl)-1H-pyrazole: To a stirred solution of (4-nitro-1-((2-(trimethylsilyl)ethoxy)methyl)-1H-pyrazol-3-yl)methyl methanesulfonate (3.6 g, 10.25 mmol) in acetonitrile (36 ml) was added 20% NaSMe (7.2 mL) at 0° C. and then stirred at room temperature for 3 h. Progress of the reaction was monitored by TLC. After completion of the reaction, the reaction mass was concentrated, diluted with ethyl acetate (30 mL) and washed with water. The organic layer was dried over Na₂SO₄, concentrated under reduced pressure to give 3-((methylthio)methyl)-4-nitro-1-((2-(trimethylsilyl)ethoxy)methyl)-1H-pyrazole (2 g, 64.5% yield) as brown liquid. The crude product was used as such for next step, without further purification. LC-MS m/z [M+H]+=304.13.

3-((methylthio)methyl)-1-((2-(trimethylsilyl)ethoxy)methyl)-1H-pyrazol-4-amine: To a stirred solution of 3-((methylthio)methyl)-4-nitro-1-((2-(trimethylsilyl)ethoxy)methyl)-1H-pyrazole (800 mg, 2.63 mmol) in ethanol (8 ml) and water (8 ml), were added NH₄Cl (1.397 g, 26.36 mmol) and iron powder (1.44 g, 26.36 mmol) and stirred at 60° C. for 2 h. Progress of the reaction was monitored by TLC. After, completion of the reaction, the reaction mass was filtered through plug of celite; the filtrate was concentrated, diluted with ethyl acetate and washed with water. The organic layer was dried over Na₂SO₄, concentrated under reduced pressure to give 3-((methylthio)methyl)-1-((2-(trimethylsilyl)ethoxy)methyl)-1H-pyrazol-4-amine (500 mg, 71.42% yield) as pale red liquid. The crude product was used as such for next step without further purification. LC-MS m/z [M+H]+=274.31.

As per the method described in Example 2, coupling to Intermediate 1 and deprotection of SEM provided for

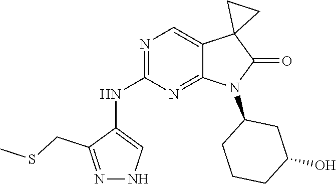 [SMILES: CSCc1n[nH]cc1Nc1ncc2c(n1)N([C@@H]1CCC[C@@H](O)C1)C(=O)C21CC1]

7′-((1R,3R)-3-hydroxycyclohexyl)-2′-((3-((methylthio)methyl)-1H-pyrazol-4-yl)amino)spiro[cyclopropane-1,5′-pyrrolo[2,3-d]pyrimidin]-6′(7′H)-one

### Example 69

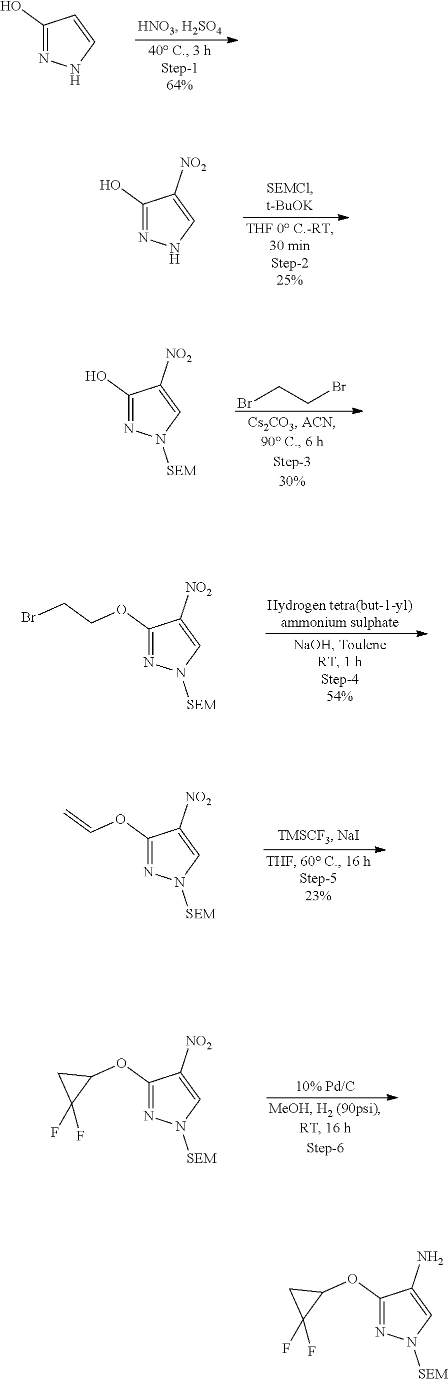

4-nitro-1H-pyrazol-3-ol: H₂SO₄ (300 mL, 5.629 mol) was cooled to −10° C. and added 1H-pyrazol-3-ol (100 g, 1.204 mol), followed by HNO₃ (100 mL, 1.95 mol). The mixture was stirred at 40° C. for 3 h. The progress of the reaction was monitored by TLC and LCMS. The reaction mixture was quenched with ice cold water (200 mL), precipitation was observed. Filtered the precipitated solid and dried under vacuum to get the crude compound. The crude was purified by manual column using 100-200 silica gel compound was eluted with 100% ethyl acetate to give 4-nitro-1H-pyrazol-3-ol as a Pale-yellow solid. (99 g, 64% yield). LC-MS m/z [M+H]⁺=130.16

Synthesis of 4-nitro-1-((2-(trimethylsilyl) ethoxy) methyl)-1H-pyrazol-3-ol: To a stirred solution of 4-nitro-1H-pyrazol-3-ol (50 g, 387.4 mmol) in THF (500 mL) at 0° C. was added t-BuOK (21.73 g, 193.7 mmol), followed by SEM-Cl (61.7 mL, 348.6 mmol). The reaction mixture was stirred at RT for 30 min. The progress of the reaction was monitored by TLC and LCMS. After completion of the reaction, the reaction mixture was quenched with water (100 mL) and extracted with ethyl acetate (4×200 mL). The combined organic layer was dried over sodium sulphate, filtered and evaporated under reduced pressure to give crude product. The crude was purified by column using 100-200 silica gel, compound was eluted with 30% ethyl acetate in pet-ether to give 4-nitro-1-((2-(trimethylsilyl) ethoxy) methyl)-1H-pyrazol-3-ol as a Yellow solid (30 g, 25% yield). ¹H NMR (400 MHz, CDCl₃) δ=8.229 (br-s, 1H), 8.109 (s, 1H), 5.29 (s, 2H), 3.64-3.68 (m, 2H), 0.92-0.96 (m, 2H), −0.00 (s, 9H).

3-(2-bromoethoxy)-4-nitro-1-((2-(trimethylsilyl) ethoxy) methyl)-1H-pyrazole: To a stirred solution of 4-nitro-1-((2-(trimethylsilyl) ethoxy) methyl)-1H-pyrazol-3-ol (10 g, 38.6 mmol) in ACN (150 mL) at 0° C. was added Cs₂CO₃ (31.4 g, 96.4 mmol), 1,2-dibromoethane (10.9 g, 57.8 mmol). The reaction mixture was stirred at 90° C. for 6 h. The progress of the reaction was monitored by TLC and LCMS. After completion of the reaction, reaction mixture was filtered through celite pad, filtrate was evaporated under reduced pressure to give crude compound, which was purified by Sepa-Bean using silica gel (230-400 mesh), eluting with 15-20% ethyl acetate in pet-ether to give 4-nitro-1-((2-(trimethylsilyl) ethoxy) methyl)-1H-pyrazol-3-ol as an Off-white solid (5.1 g, 30% yield). LC-MS m/z [M+H]⁺=366.34.

4-nitro-1-((2-(trimethylsilyl) ethoxy) methyl)-3-(vinyloxy)-1H-pyrazole: To a stirred solution of 4-nitro-1-((2-(trimethylsilyl) ethoxy) methyl)-1H-pyrazol-3-ol (4.1 g, 11.19 mmol) in Toluene (82 mL) at 0° C. was added 50% NaOH solution (22.5 mL), Hydrogen tetra(but-1-yl) ammonium sulphate (3.801 g, 11.19 mmol). The reaction mixture was stirred at RT for 1 h. The progress of the reaction was monitored by TLC and LCMS. After completion of the reaction, reaction mixture was quenched with water (50 mL) and extracted with ethyl acetate (2×50 mL). The organic layer was dried over sodium sulphate, filtered and concentrated under reduced pressure to give crude product, which was purified by Sepa-Bean using silica gel (230-400 mesh), compound was eluted with 25-30% ethyl acetate in pet-ether to give 4-nitro-1-((2-(trimethylsilyl) ethoxy) methyl)-3-(vinyloxy)-1H-pyrazole as an Off-white solid (2.0 g, 54% yield). ¹H NMR (400 MHz, CDCl₃) δ=8.185 (s, 1H), 7.12-7.25 (m, 1H), 5.27 (s, 2H), 5.07-5.11 (m, 1H), 4.63-4.66 (m, 1H), 3.61-3.65 (m, 2H), 0.92-0.96 (m, 2H), −0.00 (s, 9H).

3-(2,2-difluorocyclopropoxy)-4-nitro-1-((2-(trimethylsilyl) ethoxy) methyl)-1H-pyrazole: To a stirred solution of 4-nitro-1-((2-(trimethylsilyl) ethoxy) methyl)-3-(vinyloxy)-1H-pyrazole (550 mg, 1.92 mmol) in THE (5.5 mL) at 0° C. was added NaI (144 mg, 0.964 mmol), TMSCF₃ (5.69 mL 38.54 mmol). The reaction mixture was stirred at 60° C. for 16 h. The progress of the reaction was monitored by TLC and LCMS. After completion of the reaction, reaction mixture was quenched with water (10 mL) and extracted with ethyl acetate (2×10 mL). The organic layer was dried over sodium sulphate, filtered and evaporated under reduced pressure to give crude product. The crude was purified by column using 100-200 silica gel, compound was eluted with 25-30% ethyl acetate in pet-ether to give 3-(2,2-difluorocyclopropoxy)-4-nitro-1-((2-(trimethylsilyl) ethoxy) methyl)-1H-pyrazole as an Off-white solid (150 mg, 23% yield).

3-(2,2-difluorocyclopropoxy)-1-((2-(trimethylsilyl) ethoxy) methyl)-1H-pyrazol-4-amine: To a stirred solution of 3-(2,2-difluorocyclopropoxy)-4-nitro-1-((2-(trimethylsilyl) ethoxy) methyl)-1H-pyrazole (670 mg, 2.0 mmol) in MeOH (10 mL) was added 10% Pd/C (200 mg, 30%). The reaction mixture was stirred under H₂ (90 psi) at room temperature for 16 h. The progress of the reaction was monitored by TLC and LCMS. The reaction mixture was filtered through celite pad; filtrate was evaporated under reduced pressure to give 3-(2,2-difluorocyclopropoxy)-1-((2-(trimethylsilyl) ethoxy) methyl)-1H-pyrazol-4-amine as a Brown liquid (550 mg). LC-MS m/z [M+H]⁺=306.16.

As per the method described in Example 2, coupling to Intermediate 1 and deprotection of SEM followed by chiral SFC provided for Peak 1

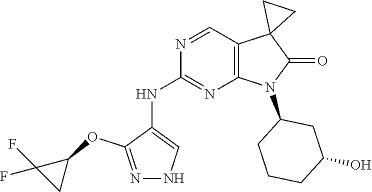 [SMILES: O=C1N([C@@H]2CCC[C@@H](O)C2)c2nc(Nc3c[nH]nc3O[C@H]3CC3(F)F)ncc2C12CC2]

2′-((3-((S-2,2 difluorocyclopropoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

### Example 70

Peak 2 from Chiral SFC in example 69 provided for

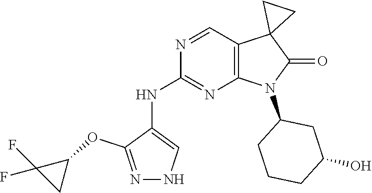 [SMILES: O=C1N([C@@H]2CCC[C@@H](O)C2)c2nc(Nc3c[nH]nc3O[C@@H]3CC3(F)F)ncc2C12CC2]

2′-((3-((R)-2,2-difluorocyclopropoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

### Example 71

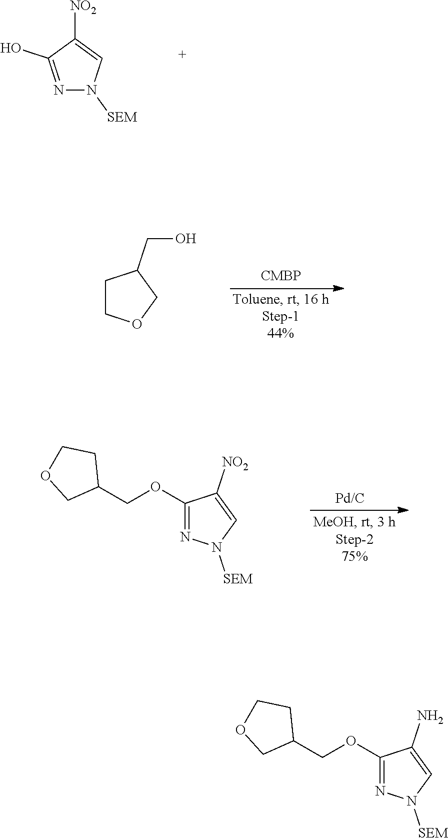 [SMILES: C[Si](C)(C)CCOCn1cc([N+](=O)[O-])c(O)n1 ; OCC1CCOC1 ; C[Si](C)(C)CCOCn1cc([N+](=O)[O-])c(OCC2CCOC2)n1 ; C[Si](C)(C)CCOCn1cc(N)c(OCC2CCOC2)n1]

4-nitro-3-((tetrahydrofuran-3-yl)methoxy)-1-((2-(trimethylsilyl)ethoxy)methyl)-1H-pyrazole: To a stirred solution of 4-nitro-1-((2-(trimethylsilyl)ethoxy)methyl)-1H-pyrazol-3-ol (1 g, 3.85 mmol) and (tetrahydrofuran-3-yl)methanol (0.59 g, 5.78 mmol) in dry toluene (20 mL), was added CMBP (1.86 g, 7.71 mmol) and stirred at room temperature for 16 h. Progress of the reaction was monitored by TLC and LCMS. After completion of the reaction, the reaction mass was diluted with ethyl acetate, washed with water and brine solution. The organic layer was dried over Na₂SO₄, concentrated under reduced pressure to give crude compound. The crude compound was purified by flash column chromatography (24 g, silica gel column), eluting with 0-30% ethyl acetate in pet-ether to give 4-nitro-3-((tetrahydrofuran-3-yl)methoxy)-1-((2-(trimethylsilyl)ethoxy)methyl)-1H-pyrazole (0.7 g, 44% yield) as pale yellow liquid. LC-MS m/z [M+H]⁺=344.43

3-((tetrahydrofuran-3-yl)methoxy)-1-((2-(trimethylsilyl)ethoxy)methyl)-1H-pyrazol-4-amine: To a stirred solution of 4-nitro-3-((tetrahydrofuran-3-yl)methoxy)-1-((2-(trimethylsilyl)ethoxy)methyl)-1H-pyrazole (0.65 g, 1.89 mmol) in methanol (10 mL), was added Pd/C (0.2 g, 1.89 mmol) and stirred under hydrogen balloon pressure at room temperature for 3 h. Progress of the reaction was monitored by TLC and LCMS. After completion of the reaction, the reaction mass was filtered, filtrate was concentrated under reduced pressure to give crude 3-((tetrahydrofuran-3-yl)methoxy)-1-((2-(trimethylsilyl)ethoxy)methyl)-1H-pyrazol-4-amine (0.45 g, 75% yield) as maroon liquid. ¹H NMR (400 MHz, DMSO-d₆) δ=7.082 (s, 1H), 5.087 (s, 2H), 4.100-3.973 (m, 2H), 3.805-3.756 (m, 2H), 3.706-3.669 (m, 1H), 3.589-3.568 (m, 2H), 3.576-3.499 (m, 2H), 2.689-2.554 (m, 1H), 2.005-1.954 (m, 1H), 1.677-1.685 (m, 1H), 0.855-0.815 (m, 2H), 0.000 (s, 9H).

As per the method described in Example 2, coupling to Intermediate 1 and deprotection of SEM followed by chiral SFC afforded Peak 1,

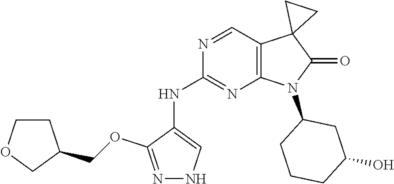 [SMILES: O=C1N([C@@H]2CCC[C@@H](O)C2)c2nc(Nc3c[nH]nc3OC[C@@H]3CCOC3)ncc2C12CC2]

7′-((1R,3R)-3-hydroxycyclohexyl)-2′-((3-(((R)-tetrahydrofuran-3-yl)methoxy)-1H-pyrazol-4-yl)amino)spiro[cyclopropane-1,5′-pyrrolo[2,3-d]pyrimidin]-6′(7′H)-one

### Example 72

Peak 2 from Chiral SFC of example 71 afforded

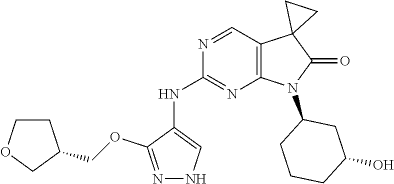 [SMILES: O=C1N([C@@H]2CCC[C@@H](O)C2)c2nc(Nc3c[nH]nc3OC[C@H]3CCOC3)ncc2C12CC2]

7′-((1R,3R)-3-hydroxycyclohexyl)-2′-((3-(((S)-tetrahydrofuran-3-yl)methoxy)-1H-pyrazol-4-yl)amino)spiro[cyclopropane-1,5′-pyrrolo[2,3-d]pyrimidin]-6′(7′H)-one

### Example 73

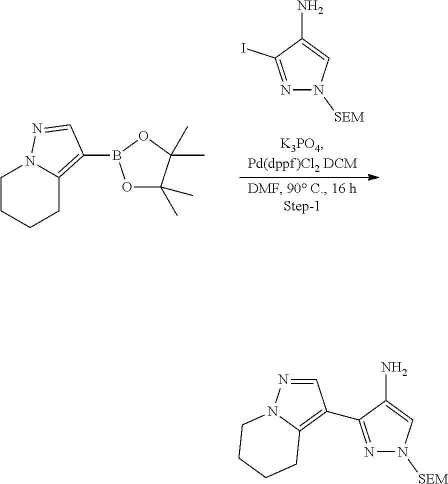 [SMILES: C[Si](C)(C)CCOCn1cc(N)c(I)n1 ; CC1(C)OB(c2cnn3c2CCCC3)OC1(C)C ; C[Si](C)(C)CCOCn1cc(N)c(-c2cnn3c2CCCC3)n1]

3-(4,5,6,7-tetrahydropyrazolo[1,5-a]pyridin-3-yl)-1-((2-(trimethylsilyl)ethoxy)methyl)-1H-pyrazol-4-amine: To a degassed solution of 3-(4,4,5,5-tetramethyl-1,3,2-dioxaborolan-2-yl)-4,5,6,7-tetrahydropyrazolo[1,5-a]pyridine (0.2 g, 0.802 mmol) in DMF (4 mL), were added 3-iodo-1-((2-(trimethylsilyl)ethoxy)methyl)-1H-pyrazol-4-amine (0.22 g, 0.66 mmol), K₃PO₄ (0.35 g, 1.67 mmol) and Pd(dppf)Cl₂·DCM (0.054 g, 0.06 mmol), stirred at 90° C. for 16 h. Progress of the reaction was monitored by TLC and LCMS. After completion of the reaction, the reaction mass was concentrated under reduced pressure to give crude compound. The crude compound was purified by flash column chromatography (12 g, silica gel column), eluting with 0-5% MeOH in DCM to give 3-(4,5,6,7-tetrahydropyrazolo[1,5-a]pyridin-3-yl)-1-((2-(trimethylsilyl)ethoxy)methyl)-1H-pyrazol-4-amine (0.2 g, 42% yield) as pale brown solid. LC-MS m/z [M+H]⁺=334.46

As per the method described in Example 2, coupling to Intermediate 1 and deprotection of SEM provided for

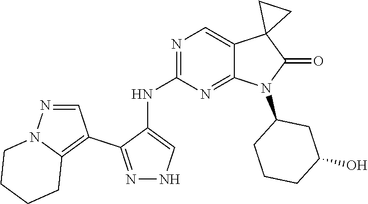 [SMILES: O=C1N([C@@H]2CCC[C@@H](O)C2)c2nc(Nc3c[nH]nc3-c3cnn4c3CCCC4)ncc2C12CC2]

7′-((1R,3R)-3-hydroxycyclohexyl)-2′-((3-(4,5,6,7-tetrahydropyrazolo[1,5-a]pyridin-3-yl)-1H-pyrazol-4-yl)amino)spiro[cyclopropane-1,5′-pyrrolo[2,3-d]pyrimidin]-6′(7′H)-one

### Example 74

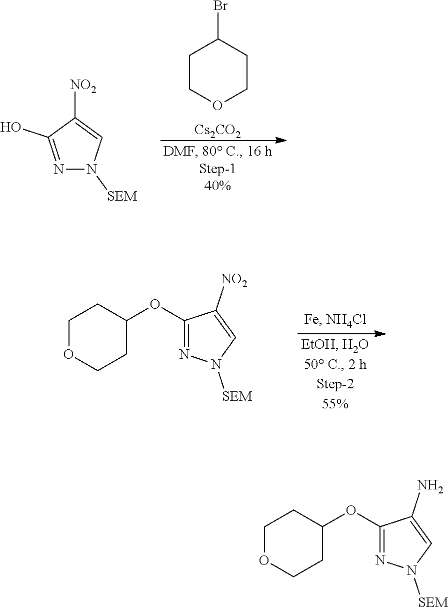

4-nitro-3-((tetrahydro-2H-pyran-4-yl)oxy)-1-((2-(trimethylsilyl)ethoxy)methyl)-1H-pyrazole: To a stirred solution of 4-nitro-1-((2-(trimethylsilyl)ethoxy)methyl)-1H-pyrazol-3-ol (1 g, 3.85 mmol) and 4-bromotetrahydro-2H-pyran (0.95 g, 5.78 mmol) in DMF (15 mL), was added Cs₂CO₃ (3 g, 2.38 mmol) and stirred at 80° C. for 16 hours. Progress of the reaction was monitored by TLC & LCMS, after completion of the reaction, the reaction mass was diluted with ice-cold water and extracted with ethyl acetate. The organic layer was dried on NaSO4, concentrated under reduced pressure to get crude. The crude product was purified by the flash column chromatography (24 g, silica column), eluted with 0-30% EtOAc in pet ether to give 4-nitro-3-((tetrahydro-2H-pyran-4-yl)oxy)-1-((2-(trimethylsilyl)ethoxy)methyl)-1H-pyrazole (0.6 g, 40.7%) as a pale-yellow solid. LC-MS m/z [M+H]+=344.18

3-((tetrahydro-2H-pyran-4-yl)oxy)-1-((2-(trimethylsilyl)ethoxy)methyl)-1H-pyrazol-4-amine: To a stirred solution of 4-nitro-3-((tetrahydro-2H-pyran-4-yl)oxy)-1-((2-(trimethylsilyl)ethoxy)methyl)-1H-pyrazole (0.6 g, 1.74 mmol) in ethanol (5 mL), water (5 mL), were added iron powder (0.48 g, 8.73 mmol) and NH₄Cl (0.46 g, 8.73 mmol) and stirred at 50° C. for 2 hours. The progress of the reaction was monitored by TLC. After completion of the reaction, the reaction mass was diluted with EtOAc and filtered on celite pad, washed with water and brine solution. The organic layer was dried over Na₂SO₄, concentrated under vacuum. The crude was purified by flash column chromatography (12 g silica column), eluting with 0-50% EtOAc in pet-ether to give 3-((tetrahydro-2H-pyran-4-yl)oxy)-1-((2-(trimethylsilyl)ethoxy)methyl)-1H-pyrazol-4-amine (0.3 g, 55%) as a brown liquid. LC-MS m/z [M+H]+=314.23

As per the method described in Example 2, coupling to Intermediate 1 and deprotection of SEM provided for

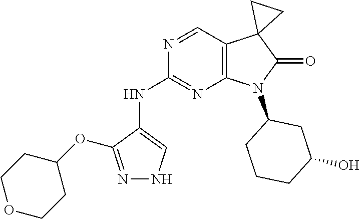 [SMILES: O=C1N([C@@H]2CCC[C@@H](O)C2)c2nc(Nc3c[nH]nc3OC3CCOCC3)ncc2C12CC2]

7′-((1R,3R)-3-hydroxycyclohexyl)-2′-((3-((tetrahydro-2H-pyran-4-yl)oxy)-1H-pyrazol-4-yl)amino)spiro[cyclopropane-1,5′-pyrrolo[2,3-d]pyrimidin]-6′(7′H)-one

### Example 75

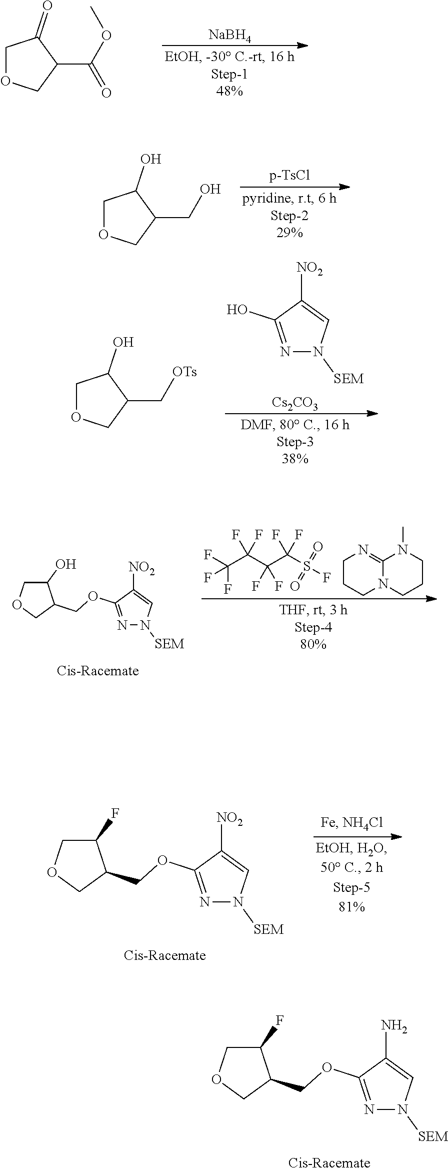

4-(hydroxymethyl)tetrahydrofuran-3-ol: To a stirred solution of NaBH₄ (6.56 g, 173.5 mmol) in ethanol (20 mL), was added methyl 4-oxotetrahydrofuran-3-carboxylate (5 g, 34.69 mmol) as a solution in ethanol (20 mL) at −30° C. The resulting reaction mass was stirred at −30° C. for 30 min and then allowed to stir at room temperature for 16 h. Progress of the reaction was monitored by TLC. After completion of the reaction, the reaction mixture was acidified with 1N aq HCl. All the solvents were evaporated under vacuum. Solid was triturated with 10% MeOH in DCM and decanted. The decanted portions were concentrated under vacuum to get crude product. The crude product was purified by the flash column chromatography (40 g, silica column), eluting with 0-5% MeOH in DCM to get 4-(hydroxymethyl)tetrahydrofuran-3-ol (2 g, 48% yield) as a pale-yellow semi-solid. 1H NMR (400 MHz, CDCl3) δ=4.53 (s, 1H), 4.32 (brs, 2H), 4.09-4.11 (m, 2H), 3.71-3.94 (m, 16H), 2.87 (m, 1H), 2.22-2.75 (m, 8H).

(4-hydroxytetrahydrofuran-3-yl)methyl 4-methylbenzenesulfonate: To a stirred solution of 4-(hydroxymethyl)tetrahydrofuran-3-ol (2.7 g, 22.86 mmol) in pyridine (27.00 mL) was added p-TsCl (4.793 g, 25.14 mmol) and stirred at room temperature for 6 h. Progress of the reaction was monitored by TLC. After completion of the reaction, the reaction was diluted with ethyl acetate washed with water. Combined organic layers were dried over, Na₂SO₄, concentrated under vacuum to get crude. The crude product was purified by the flash column chromatography (24 g, silica column), eluted with 0-5% MeOH in DCM. to get (4-hydroxytetrahydrofuran-3-yl)methyl 4-methylbenzenesulfonate (1.8 g, 29%) as a colourless semi-solid. LC-MS m/z [M+H]+=273.20

4-(((4-nitro-1-((2-(trimethylsilyl)ethoxy)methyl)-1H-pyrazol-3-yl)oxy)methyl)tetrahydrofuran-3-ol: To a stirred solution of 4-nitro-1-((2-(trimethylsilyl)ethoxy)methyl)-1H-pyrazol-3-ol (1.6 g, 6.208 mmol) and (4-hydroxytetrahydrofuran-3-yl)methyl 4-methylbenzenesulfonate (1.69 g, 6.208 mmol) in DMF (30.000 mL) was added Cs₂CO₃ (4 g, 12.42 mmol) and stirred at 80° C. for 16 h. Progress of the reaction was monitored by TLC and LCMS. After completion of the reaction, the reaction mass was quenched with ice-cold water, extracted with ethyl acetate. The organic layer was dried over Na₂SO₄, concentrated under reduced pressure to get crude. The crude product was purified by the flash column chromatography (24 g, silica column), eluted with 0-60% EtOAc in pet ethee to give 4-(((4-nitro-1-((2-(trimethylsilyl)ethoxy)methyl)-1H-pyrazol-3-yl)oxy)methyl)tetrahydrofuran-3-ol (800 mg, 38% yield) as off white solid. LC-MS m/z [M+H]+=360.46

3-(((4-fluorotetrahydrofuran-3-yl)methoxy)-4-nitro-1-((2-(trimethylsilyl)ethoxy)methyl)-1H-pyrazole: To a stirred solution of 4-(((4-nitro-1-((2-(trimethylsilyl)ethoxy)methyl)-1H-pyrazol-3-yl)oxy)methyl)tetrahydrofuran-3-ol (1 g, 2.782 mmol) in THE (10 mL), were added PBSF (1.26 g, 4.173 mmol) as a solution in THE (1 mL), 1-methyl-1,3,4,6,7,8-hexahydro-2H-pyrimido[1,2-a]pyrimidine (0.64 g 4.17 mmol) and stirred at room temperature for 3 h. Progress of the reaction was monitored by TLC and LCMS. After completion of the reaction, the reaction mass was concentrated under vacuum to get crude. The crude product was purified by the flash column chromatography (24 g, silica column), eluted with 0-30% EtOAc in pet ether to give 3-((-4-fluorotetrahydrofuran-3-yl)methoxy)-4-nitro-1-((2-(trimethylsilyl)ethoxy)methyl)-1H-pyrazole (800 mg, 80%) as a colourless solid. LC-MS m/z [M+H]+=362.48

3-((-4-fluorotetrahydrofuran-3-yl)methoxy)-1-((2-(trimethylsilyl)ethoxy)methyl)-1H-pyrazol-4-amine: To a stirred solution of 3-((-4-fluorotetrahydrofuran-3-yl)methoxy)-4-nitro-1-((2-(trimethylsilyl)ethoxy)methyl)-1H-pyrazole (0.8 g, 2.21 mmol) in EtOH (1 mL) and water (1 mL) were added Iron powder (0.61 g, 11.06 mmol), NH₄Cl (0.59 g, 11.06 mmol) and stirred at 50° C. for 2 h. Progress of the reaction was monitored by TLC. After completion of the reaction, the reaction mass was diluted with EtOAc and filtered on celite pad, washed with EtOAc. The filtrate was concentrated under vacuum to get the crude.

The crude product was purified by the flash column chromatography (12 g silica column) eluted with 0-50% EtOAc in pet ether to give cis-racemate 3-((-4-fluorotetrahydrofuran-3-yl)methoxy)-1-((2-(trimethylsilyl)ethoxy)methyl)-1H-pyrazol-4-amine (0.6 g, 81% yield) as a yellow semi-solid. LC-MS m/z [M+H]+=332.48

As per the method described in Example 2, coupling to Intermediate 1 and deprotection of SEM provided for cis-racemate

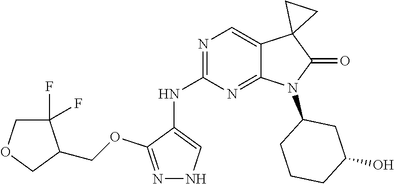 [SMILES: O=C1N([C@@H]2CCC[C@@H](O)C2)c2nc(Nc3c[nH]nc3OCC3COCC3(F)F)ncc2C12CC2]

2′-((3-((-4-fluorotetrahydrofuran-3-yl)methoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

### Example 76

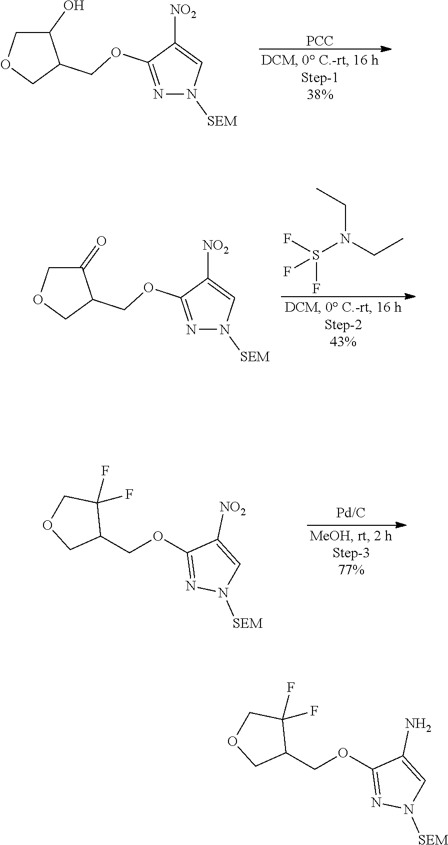

4-(((4-nitro-1-((2-(trimethylsilyl)ethoxy)methyl)-1H-pyrazol-3-yl)oxy)methyl)dihydrofuran-3(2H)-one: To a stirred solution of 4-(((4-nitro-1-((2-(trimethylsilyl)ethoxy)methyl)-1H-pyrazol-3-yl)oxy)methyl)tetrahydrofuran-3-ol (2 g, 5.56 mmol) in DCM (30 mL) was added PCC (1.79 g, 8.34 mmol) at 0° C. and then stirred at room temperature for 16 h. Progress of the reaction was monitored by TLC and LCMS. After completion of the reaction, the reaction mixture was diluted with water and extracted with DCM. The combined organic layers were dried over Na₂SO₄, concentrated under reduced pressure to get crude. The crude product was purified by the flash column chromatography (40 g, silica gel column), eluted with 0-30% EtOAc in pet ether to get the pure 4-(((4-nitro-1-((2-(trimethylsilyl)ethoxy)methyl)-1H-pyrazol-3-yl)oxy)methyl)dihydrofuran-3(2H)-one (1.2 g, 38.67% yield) as colourless semi-solid. LC-MS m/z [M+H]⁺=358.49

3-((4,4-difluorotetrahydrofuran-3-yl)methoxy)-4-nitro-1-((2-(trimethylsilyl)ethoxy)methyl)-1H-pyrazole: To a stirred solution of 4-(((4-nitro-1-((2-(trimethylsilyl)ethoxy)methyl)-1H-pyrazol-3-yl)oxy)methyl)dihydrofuran-3(2H)-one (1.1 g, 3.07 mmol) in DCM (20 mL) was added DAST (2.48 g, 15.39 mmol) at 0° C., and then stirred at room temperature for 16 h. Progress of the reaction was monitored by TLC and LCMS. After completion of the reaction, the reaction mass was diluted with Ice-cold aq. NaHCO₃, extracted with DCM. The organic layer was dried over Na₂SO₄, concentrated under reduced pressure to get crude product. The crude product was purified by the flash column chromatography (24 g, silica column), eluted with 0-40% EtOAc in pet ether to get (R)-3-((4,4-difluorotetrahydrofuran-3-yl)methoxy)-4-nitro-1-((2-(trimethylsilyl)ethoxy)methyl)-1H-pyrazole (0.5 g, 43% yield) as a colourless semi-solid. LC-MS m/z [M+H]+=380.46.

3-((4,4-difluorotetrahydrofuran-3-yl)methoxy)-1-((2-(trimethylsilyl)ethoxy)methyl)-1H-pyrazol-4-amine: To a stirred solution of (R)-3-((4,4-difluorotetrahydrofuran-3-yl)methoxy)-4-nitro-1-((2-(trimethylsilyl)ethoxy)methyl)-1H-pyrazole (0.4 g, 1.05 mmol) in methanol (8 mL) was added Pd—C (0.22 g, 1.26 mmol) and stirred at room temperature for 2 h under hydrogen bladder pressure. The progress of the reaction was monitored by TLC. After completion of reaction, the reaction mass was diluted with MeOH and filtered on celite pad, the filtrate was concentrated under reduced pressure to get the crude product. The crude product was purified by the flash column chromatography (12 g, silica column), eluted with 0-5% MeOH in DCM to get the (R)-3-((4,4-difluorotetrahydrofuran-3-yl)methoxy)-1-((2-(trimethylsilyl)ethoxy)methyl)-1H-pyrazol-4-amine (280 mg, 77% yield) as pale yellow semi-solid. LC-MS m/z [M+H]⁺=350.46

As per the method described in Example 2, coupling to Intermediate 1 and deprotection of SEM provided for

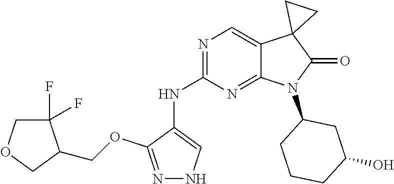 [SMILES: O=C1N([C@@H]2CCC[C@@H](O)C2)c2nc(Nc3c[nH]nc3OCC3COCC3(F)F)ncc2C12CC2]

2′-((3-((4,4-difluorotetrahydrofuran-3-yl)methoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

### Example 77

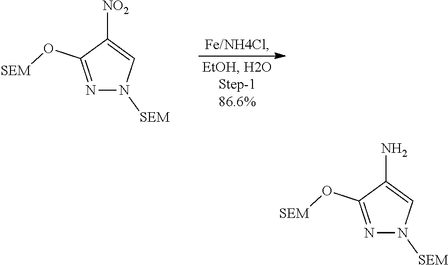 [SMILES: C[Si](C)(C)CCOCOc1nn(COCC[Si](C)(C)C)cc1[N+](=O)[O-] ; C[Si](C)(C)CCOCOc1nn(COCC[Si](C)(C)C)cc1N]

To a stirred solution of 4-nitro-3-((2-(trimethylsilyl)ethoxy)methoxy)-1-((2-(trimethylsilyl)ethoxy)methyl)-1H-pyrazole (1.0 g, 2.567 mmol) in ethanol (10 ml) and water (10 ml), were added NH₄Cl (1.373 g, 25.67 mmol) and iron powder (1.434 g, 25.67 mmol) at room temperature and then stirred at 60° C. for 2 h. Progress of the reaction was monitored by TLC. After, completion of the reaction, the reaction mass was filtered through plug of celite; the filtrate was concentrated, diluted with ethyl acetate (20 mL) and washed with water (2×30 mL). The organic layer was dried over Na₂SO₄, concentrated under reduced pressure to give the crude product of 3-((2-(trimethylsilyl) ethoxy) methoxy)-1-((2-(trimethylsilyl)ethoxy)methyl)-1H-pyrazol-4-amine as brown liquid (800.0 mg, 86.67% yield). ¹H NMR (400 MHz, CDCl₃) δ=7.05 (br-s, 1H), 5.32-5.42 (br-s, 2H), 5.12 (br-s, 2H), 3.47-3.82 (m, 5H), 2.82 (br-s, 1H), 0.86-0.98 (m, 4H), 0.0 (s, 18H).

As per the method described in Example 2, coupling to Intermediate 1 and deprotection of SEM provided for

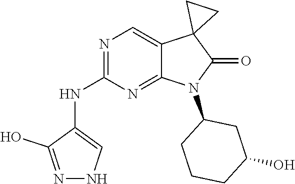 [SMILES: O=C1N([C@@H]2CCC[C@@H](O)C2)c2nc(Nc3c[nH]nc3O)ncc2C12CC2]

2′-((3-hydroxy-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one.

### Example 78

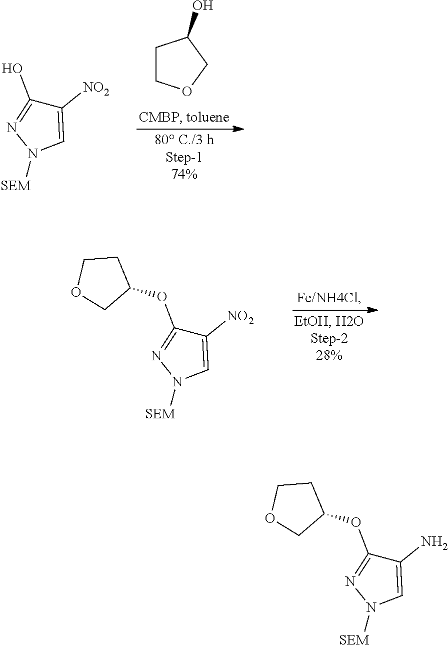 [SMILES: C[Si](C)(C)CCOCn1cc([N+](=O)[O-])c(O)n1 ; O[C@@H]1CCOC1 ; C[Si](C)(C)CCOCn1cc([N+](=O)[O-])c(O[C@H]2CCOC2)n1 ; C[Si](C)(C)CCOCn1cc(N)c(O[C@H]2CCOC2)n1]

(S)-4-nitro-3-((tetrahydrofuran-3-yl)oxy)-1-((2-(trimethylsilyl)ethoxy)methyl)-1H-pyrazole: To a stirred solution of 4-nitro-1-((2-(trimethylsilyl)ethoxy)methyl)-1H-pyrazol-3-ol (600.0 mg, 2.31 mmol) in toluene (6 ml) was added (R)-tetrahydrofuran-3-ol (305.8 mg, 3.47 mmol) and CMBP (7.243 g, 135.4 mmol) at room temperature and then stirred at 80° C. for 3 h. Progress of the reaction was monitored by TLC. After completion of the reaction, the reaction mass was diluted with, water (20 mL) and extracted with Ethyl acetate (2×30 mL). The organic layer was dried over Na₂SO₄, concentrated under reduced pressure to give the crude product. The crude compound was purified by Sepa-Bean using silica gel (230-400 mesh), eluting with 25-30% EtOAc in pet ether to give (S)-4-nitro-3-((tetrahydrofuran-3-yl)oxy)-1-((2-(trimethylsilyl)ethoxy)methyl)-1H-pyrazole as brown colour gummy solid (600 mg, 74% yield). LC-MS m/z [M+H]+=330.46.

(S)-3-((tetrahydrofuran-3-yl)oxy)-1-((2-(trimethylsilyl)ethoxy)methyl)-1H-pyrazol-4-amine: To a stirred solution of (S)-4-nitro-3-((tetrahydrofuran-3-yl)oxy)-1-((2-(trimethylsilyl)ethoxy)methyl)-1H-pyrazole (500.0 mg, 1.51 mmol) in ethanol (6 ml) and water (6 ml), were added NH₄Cl (811.9 mg, 15.1 mmol) and iron powder (847.7 mg, 15.1 mmol) at room temperature and then stirred at 60° C. for 2 h. Progress of the reaction was monitored by TLC. After, completion of the reaction, the reaction mass was filtered through plug of celite; the filtrate was concentrated, diluted with ethyl acetate (20 mL) and washed with water (2×30 mL). The organic layer was dried over Na₂SO₄, concentrated under reduced pressure to give the crude product of (S)-3-((tetrahydrofuran-3-yl)oxy)-1-((2-(trimethylsilyl)ethoxy)methyl)-1H-pyrazol-4-amine as brown liquid (350.0 mg, 28% yield). LC-MS m/z [M+H]+=300.50.

As per the method described in Example 2, coupling to Intermediate 1 and deprotection of SEM provided for

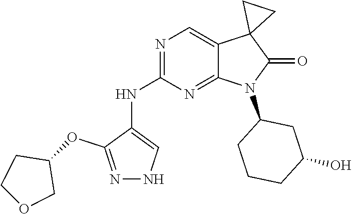 [SMILES: O=C1N([C@@H]2CCC[C@@H](O)C2)c2nc(Nc3c[nH]nc3O[C@H]3CCOC3)ncc2C12CC2]

7′-((1R,3R)-3-hydroxycyclohexyl)-2′-((3-(((S)-tetrahydrofuran-3-yl)oxy)-1H-pyrazol-4-yl)amino)spiro[cyclopropane-1,5′-pyrrolo[2,3-d]pyrimidin]-6′(7′H)-one

### Example 79

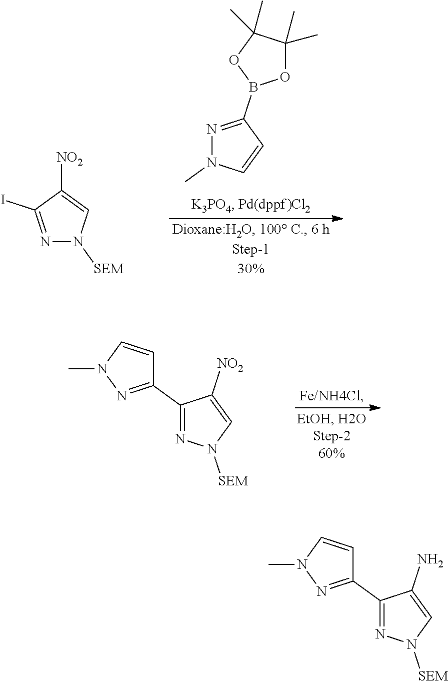

1′-methyl-4-nitro-1-((2-(trimethylsilyl)ethoxy)methyl)-1H,1′H-3,3′-bipyrazole: To a degassed solution of 3-iodo-4-nitro-1-((2-(trimethylsilyl)ethoxy)methyl)-1H-pyrazole (1.500 g, 4.063 mmol), 1-methyl-3-(4,4,5,5-tetramethyl-1,3,2-dioxaborolan-2-yl)-1H-pyrazole (1.268 g, 6.094 mmol) and K₃PO₄ (2.156 g, 10.16 mmol) in 1,4-Dioxane (15 mL) and Water (3 mL) was added Pd(dppf)Cl₂·DCM (331.8 mg, 406.3 μmol) stirred at 90° C. for 5 h. Progress of the reaction was monitored by TLC and LCMS. After completion of the reaction, the reaction mass was filtered, diluted with ethyl acetate (20 mL) and washed with water (2×30 mL). The organic layer was dried over Na₂SO₄, concentrated under reduced pressure to give the crude product. The crude compound was purified by Sepa-Bean using silica gel (230-400 mesh), eluting with 60-70% EtOAc in pet ether to give 1′-methyl-4-nitro-1-((2-(trimethylsilyl)ethoxy)methyl)-1H,1′H-3,3′-bipyrazole as brown colour gummy solid (450 mg, 30% yield). LC-MS m/z [M+H]+=324.2.

1′-methyl-1-((2-(trimethylsilyl)ethoxy)methyl)-1H,1′H-[3,3′-bipyrazol]-4-amine: To a stirred solution of 1′-methyl-4-nitro-1-((2-(trimethylsilyl)ethoxy)methyl)-1H,1′H-3,3′-bipyrazole (450.0 mg, 1.391 mmol) in ethanol (4.5 ml) and water (4.5 ml), were added NH₄Cl (744.2 mg, 13.91 mmol) and iron powder (777.1 mg, 13.91 mmol) at room temperature and then stirred at 50° C. for 3 h. Progress of the reaction was monitored by TLC. After, completion of the reaction, the reaction mass was filtered through plug of celite; the filtrate was concentrated, diluted with ethyl acetate (20 mL) and washed with water (2×30 mL). The organic layer was dried over Na₂SO₄, concentrated under reduced pressure to give the crude product of 1′-methyl-1-((2-(trimethylsilyl)ethoxy)methyl)-1H,1′H-[3,3′-bipyrazol]-4-amine as brown liquid (300.0 mg, 60% yield). LC-MS m/z [M+H]+=294.31.

As per the method described in Example 2, coupling to Intermediate 1 and deprotection of SEM provided for

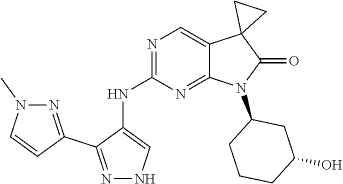 [SMILES: Cn1ccc(-c2n[nH]cc2Nc2ncc3c(n2)N([C@@H]2CCC[C@@H](O)C2)C(=O)C32CC2)n1]

7-((1R,3R)-3-hydroxycyclohexyl)-2′-((1′-methyl-1H,1′H-[3,3′-bipyrazol]-4-yl)amino)spiro[cyclopropane-1,5′-pyrrolo[2,3-d]pyrimidin]-6′(7′H)-one

### Example 80

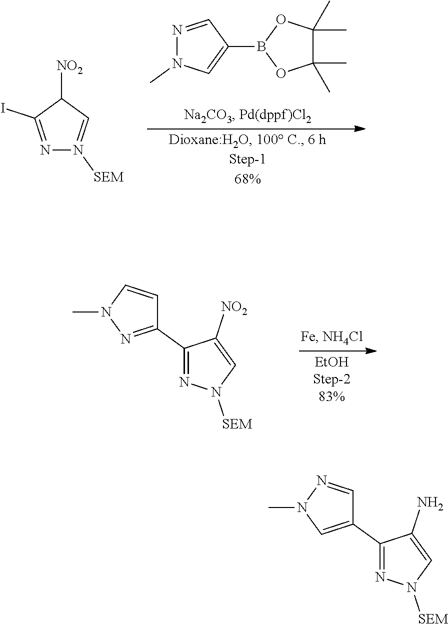

1′-methyl-1-((2-(trimethylsilyl)ethoxy)methyl)-1H,1′H-[3,4′-bipyrazol]-4-amine: To a degassed solution of 3-iodo-4-nitro-1-((2-(trimethylsilyl)ethoxy)methyl)-1H-pyrazole (1.0 g, 2.71 mmol), 1-methyl-4-(4,4,5,5-tetramethyl-1,3,2-dioxaborolan-2-yl)-1H-pyrazole (676 mg, 3.25 mmol) and Na₂CO₃ (574 mg, 5.42 mmol) in ACN (30 mL) and Water (5 mL) was added Pd(dppf)Cl₂·DCM (198.0 mg, 0.27 mmol) stirred at 90° C. for 16 h. Progress of the reaction was monitored by TLC and LCMS. After completion of the reaction, the reaction mass was filtered, diluted with ethyl acetate (20 mL) and washed with water (2×30 mL). The organic layer was dried over Na₂SO₄, concentrated under reduced pressure to give the crude product. The crude compound was purified by Sepa-Bean using silica gel (230-400 mesh), eluting with 40-50% EtOAc in pet ether to give 1′-methyl-1-((2-(trimethylsilyl)ethoxy)methyl)-1H,1′H-[3,4′-bipyrazol]-4-amine as brown colour gummy solid (600 mg, 68% yield). LC-MS m/z [M+H]+=323.4.

1′-methyl-4-nitro-1-((2-(trimethylsilyl)ethoxy)methyl)-1H,1′H-3,4′-bipyrazole: To a stirred solution of 1′-methyl-1-((2-(trimethylsilyl)ethoxy)methyl)-1H,1′H-[3,4′-bipyrazol]-4-amine (600.0 mg, 1.85 mmol) in ethanol (6.0 ml) and water (6.0 ml), were added NH₄Cl (947.3 mg, 18.57 mmol) and iron powder (984.5 mg, 18.57 mmol) at room temperature and then stirred at 50° C. for 3 h. Progress of the reaction was monitored by TLC. After, completion of the reaction, the reaction mass was filtered through plug of celite; the filtrate was concentrated, diluted with ethyl acetate (20 mL) and washed with water (2×30 mL). The organic layer was dried over Na₂SO₄, concentrated under reduced pressure to give the crude product of 1′-methyl-4-nitro-1-((2-(trimethylsilyl)ethoxy)methyl)-1H,1′H-3,4′-bipyrazole as brown liquid (450.0 mg, 83% yield). LC-MS m/z [M+H]+=293.4.

As per the method described in Example 2, coupling to Intermediate 1 and deprotection of SEM provided for

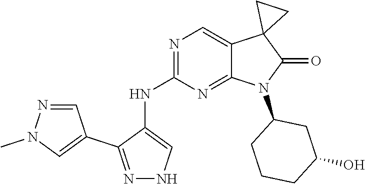 [SMILES: Cn1cc(-c2n[nH]cc2Nc2ncc3c(n2)N([C@@H]2CCC[C@@H](O)C2)C(=O)C32CC2)cn1]

7′-((1R,3R)-3-hydroxycyclohexyl)-2′-((1′-methyl-1H,1′H-[3,4′-bipyrazol]-4-yl)amino)spiro[cyclopropane-1,5′-pyrrolo[2,3-d]pyrimidin]-6′(7′H)-one

### Example 81

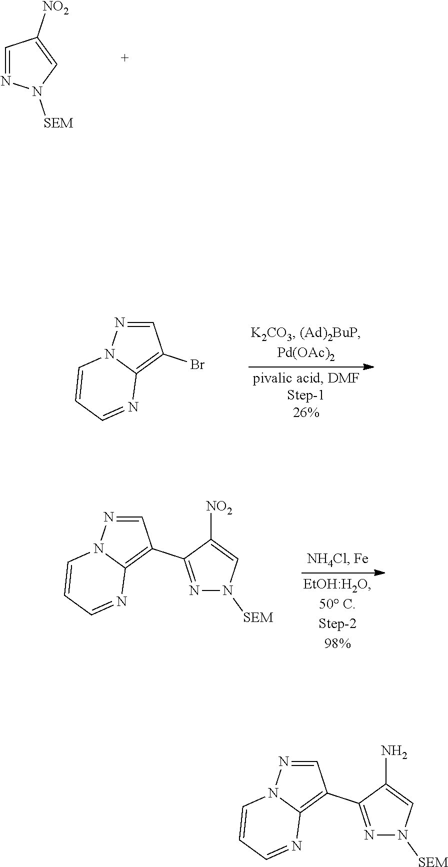 [SMILES: C[Si](C)(C)CCOCn1cc([N+](=O)[O-])cn1 ; Brc1cnn2cccnc12 ; C[Si](C)(C)CCOCn1cc([N+](=O)[O-])c(-c2cnn3cccnc23)n1 ; C[Si](C)(C)CCOCn1cc(N)c(-c2cnn3cccnc23)n1]

3-(4-nitro-1-((2-(trimethylsilyl)ethoxy)methyl)-1H-pyrazol-3-yl)pyrazolo[1,5-a]pyrimidine: To a stirred solution of 4-nitro-1-((2-(trimethylsilyl)ethoxy)methyl)-1H-pyrazole (1 g, 4.12 mmol) in DMF (20 mL) was added 3-bromopyrazolo[1,5-a]pyrimidine (1.5 g, 6.19 mmol) followed by K₂CO₃ (1.43 g, 10.31 mmol), (Ad)₂BuP (0.148 g, 0.412 mmol) and pivalic acid (0.210 g, 0.206 mmol) at RT, purged with nitrogen gas for 5 minutes and was added Pd(OAc)₂ (0.046 g, 0.206 mmol) and the resulting reaction mixture was stirred at 120° C. for 12 hours. The reaction progress was monitored by TLC and LCMS. The reaction mixture was diluted cold water and extracted with EtOAc, dried over sodium sulfate, evaporated under reduced pressure to afford crude. The crude material was absorbed onto a plug of silica gel and purified by Sepabean chromatography through a RediSep pre-packed silica gel column (24 g), eluting with a gradient of 0-50% EtOAc in pet ether, collected the pure fractions and concentrated under reduced vacuum pressure to afford 3-(4-nitro-1-((2-(trimethylsilyl)ethoxy)methyl)-1H-pyrazol-3-yl)pyrazolo[1,5-a]pyrimidine (0.4 g, 25.65% yield) as a yellow oil. LC-MS m/z [M+H]⁺=361.18

3-(pyrazolo[1,5-a]pyrimidin-3-yl)-1-((2-(trimethylsilyl)ethoxy)methyl)-1H-pyrazol-4-amine: To a stirred solution of 3-(4-nitro-1-((2-(trimethylsilyl)ethoxy)methyl)-1H-pyrazol-3-yl)pyrazolo[1,5-a]pyrimidine (300 mg, 0.832 mmol) in ethanol (3 mL) and water (3 mL) was added ammonium chloride (232 mg, 4.16 mmol) followed by iron (223 mg, 4.16 mmol) at RT and the resulting reaction mixture was stirred at 50° C. for 2 hours. The reaction progress was monitored by TLC and LCMS. After completion of reaction, all volatiles were evaporated in vacuo, the residue obtained was dissolved in dichloromethane, filtered and filtrate was evaporated in vacuo to afford 3-(pyrazolo[1,5-a]pyrimidin-3-yl)-1-((2-(trimethylsilyl)ethoxy)methyl)-1H-pyrazol-4-amine (0.35 g, 98% yield) as a brown oil. LC-MS m/z [M+H]⁺=331.45.

As per the method described in Example 2, coupling to Intermediate 1 and deprotection of SEM provided for

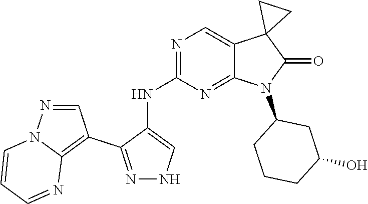 [SMILES: O=C1N([C@@H]2CCC[C@@H](O)C2)c2nc(Nc3c[nH]nc3-c3cnn4cccnc34)ncc2C12CC2]

7′-((1R,3R)-3-hydroxycyclohexyl)-2′-((3-(pyrazolo[1,5-a]pyrimidin-3-yl)-1H-pyrazol-4-yl)amino)spiro[cyclopropane-1,5′-pyrrolo[2,3-d]pyrimidin]-6′(7′H)-one

### Example 82

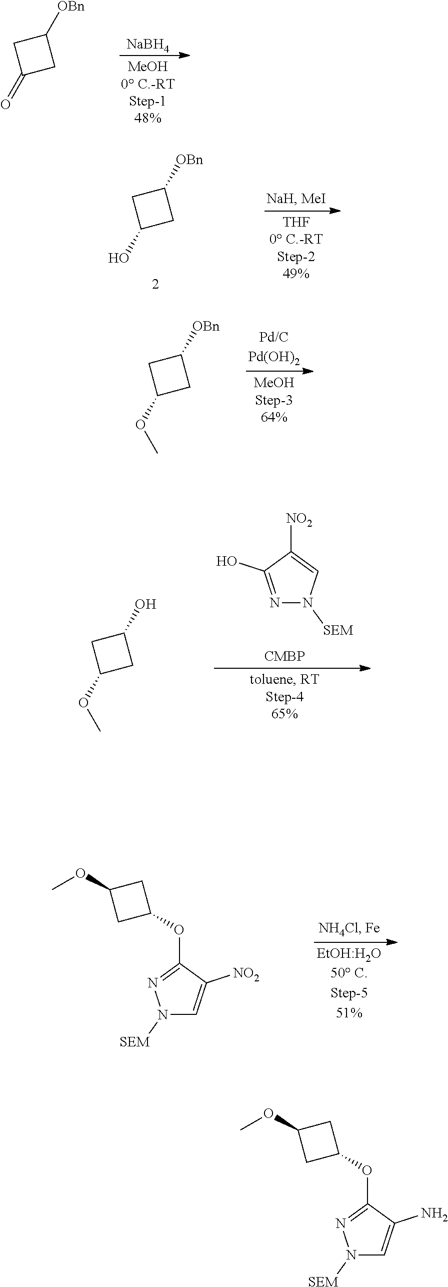

(1s,3s)-3-(benzyloxy)cyclobutan-1-ol: To a stirred solution of 3-(benzyloxy)cyclobutan-1-one (5 g, 28.37 mmol) in MeOH (50 mL) was added NaBH₄ (1.6 g, 42.56 mmol) at 0° C. and the resulting reaction mixture was stirred at RT for 2 hours. The reaction progress was monitored by TLC and LCMS. On completion, the reaction mixture was cooled to 0° C., quenched with sat. NH₄Cl solution, extracted with EtOAc, washed with brine, dried over sodium sulfate and evaporated under reduced vacuum pressure to afford (1s,3s)-3-(benzyloxy)cyclobutan-1-ol (4 g, 48% yield) as a pale yellow oil. ¹H NMR (CDCl₃): δ=7.37-7.25 (m, 5H), 4.41 (s, 2H), 3.91-3.89 (m, 1H), 3.66-3.59 (m, 1H), 2.75-2.67 (m, 2H), 1.97-1.89 (m, 2H).

(((1s,3s)-3-methoxycyclobutoxy)methyl)benzene: To a stirred solution of (1s,3s)-3-(benzyloxy)cyclobutan-1-ol (4 g, 22.44 mmol) in THE (40 mL) was added NaH (1.35 g, 60% w/w, 33.66 mmol) at 0° C., stirred for 10 minutes and was added MeI (2.1 mL, 33.66 mmol) and the resulting reaction mixture was stirred at RT for 2 hours. The reaction progress was monitored by TLC and LCMS. On completion, the reaction mixture was quenched with ice cold water (100 mL), extracted with EtOAc (2×100 mL), washed with brine (50 mL), dried over sodium sulfate and evaporated under reduced vacuum pressure to afford (((1s,3s)-3-methoxycyclobutoxy)methyl)benzene (3.5 g, 49% yield) as a pale yellow oil. ¹H NMR (CDCl₃): δ=7.36-7.25 (m, 5H), 4.42 (s, 2H), 3.71-3.64 (m, 1H), 3.53-3.46 (m, 1H), 3.23 (s, 3H), 2.66-2.59 (m, 2H), 1.97-1.89 (m, 2H).

(1s,3s)-3-methoxycyclobutan-1-ol: To a solution of (((1s,3s)-3-methoxycyclobutoxy)methyl)benzene (3.5 g, 1.93 mmol) in MeOH (35 mL) was added Pd/C (1.4 g, 40% w/w) followed by Pd(OH)₂ (0.35 g, 10% w/w) in parr shaker and the resulting reaction mixture was stirred at RT under hydrogenation pressure (60 psi) for 12 hours. The reaction progress was monitored by TLC. On completion, the reaction mixture was passed through celite pad and the pad was washed with MeOH (30 mL). Filtrate was dried over sodium sulphate, filtered and evaporated under reduced vacuum pressure to afford (1s,3s)-3-methoxycyclobutan-1-ol (1.7 g, 64% yield) as a pale yellow oil. ¹H NMR (CdCl₃): δ=4.98 (d, J=6.8 Hz, 1H), 3.68-3.66 (m, 1H), 3.36-3.32 (obs, 1H), 3.09 (s, 3H), 2.53-2.47 (m, 2H), 1.67-1.61 (m, 2H).

3-((1r,3r)-3-methoxycyclobutoxy)-4-nitro-1-((2-(trimethylsilyl)ethoxy)methyl)-1H-pyrazole: To a stirred solution of 4-nitro-1-((2-(trimethylsilyl)ethoxy)methyl)-1H-pyrazol-3-ol (500 mg, 1.93 mmol) in toluene (1 mL) was added (1s,3s)-3-methoxycyclobutan-1-ol (0.36 g, 3.47 mmol) followed by CMBP (1 mL, 3.86 mmol) and the resulting reaction mixture was stirred at RT for 12 hours. The reaction progress was monitored by TLC and LCMS. On completion, the reaction mixture was diluted with water (10 mL) and extracted with ethyl acetate. Combined organic layers was washed with brine, dried over sodium sulphate, filtered and evaporated under reduced vacuum pressure to give crude product. The crude material was absorbed onto a plug of silica gel and purified by SepaBean chromatography through a RediSep pre-packed silica gel column (24 g), eluting with a gradient of 0-30% EtOAc in pet ether, collected the pure fractions and concentrated under reduced vacuum pressure to afford 3-((1r,3r)-3-methoxycyclobutoxy)-4-nitro-1-((2-(trimethylsilyl)ethoxy)methyl)-1H-pyrazole (0.45 g, 65% yield) as a pale yellow oil. LC-MS m/z [M+H]⁺=344.21 [M+H]⁺

3-((1r,3r)-3-methoxycyclobutoxy)-1-((2-(trimethylsilyl)ethoxy)methyl)-1H-pyrazol-4-amine: To a stirred solution of 3-((1r,3r)-3-methoxycyclobutoxy)-4-nitro-1-((2-(trimethylsilyl)ethoxy)methyl)-1H-pyrazole (400 mg, 1.16 mmol) in ethanol (4 mL) and water (4 mL) was added ammonium chloride (311 mg, 5.82 mmol) followed by iron (325 mg, 5.82 mmol) and the resulting reaction mixture was stirred at 50° C. for 2 hours. The reaction progress was monitored by TLC and LCMS. After completion of reaction, all volatiles were evaporated in vacuo, the residue obtained was dissolved in dichloromethane (15 mL), filtered and filtrate was evaporated in vacuo to afford 3-((1r,3r)-3-methoxycyclobutoxy)-1-((2-(trimethylsilyl)ethoxy)methyl)-1H-pyrazol-4-amine (0.3 g, 51% yield) as a brown oil. LC-MS m/z [M+H]⁺=314.21

As per the method described in Example 2, coupling to Intermediate 1 and deprotection of SEM provided for

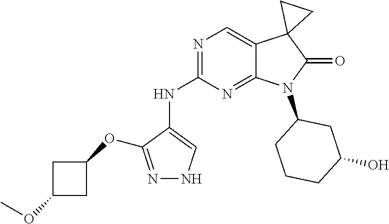 [SMILES: CO[C@H]1C[C@H](Oc2n[nH]cc2Nc2ncc3c(n2)N([C@@H]2CCC[C@@H](O)C2)C(=O)C32CC2)C1]

7′-((1R,3R)-3-hydroxycyclohexyl)-2′-((3-((1r,3R)-3-methoxycyclobutoxy)-1H-pyrazol-4-yl)amino)spiro[cyclopropane-1,5′-pyrrolo[2,3-d]pyrimidin]-6′(7′H)-one

### Example 83

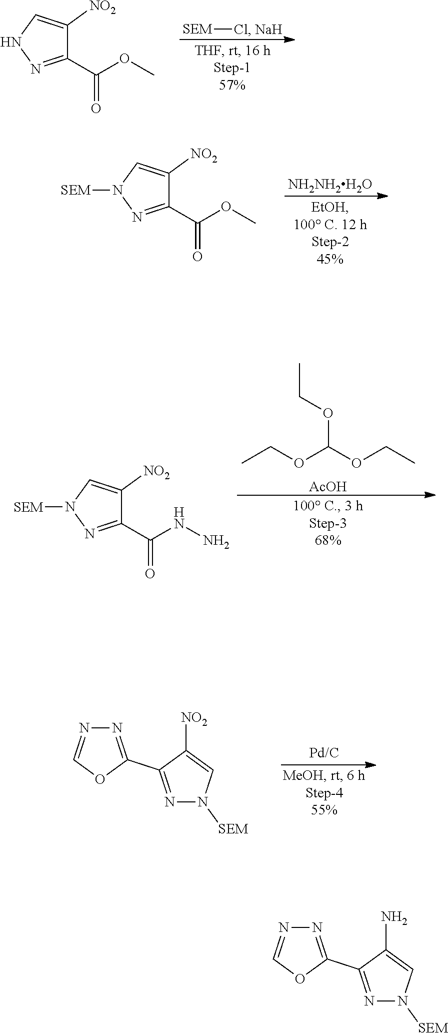

methyl 4-nitro-1-((2-(trimethylsilyl)ethoxy)methyl)-1H-pyrazole-3-carboxylate: To a stirred solution of methyl 4-nitro-1H-pyrazole-3-carboxylate (3 g, 17.53 mmol) and NaH (0.54 g, 23.99 mmol) in THE (30 mL) was added SEM-Cl (3.5 g, 21.03 mmol) at 0° C. and then stirred at room temperature for 16 h. Progress of the reaction was monitored by TLC and LCMS. After completion of the reaction, the reaction mixture was quenched with water and extracted with ethyl acetate. The combined organic layers were dried over Na₂SO₄, concentrated under reduced pressure. The crude product was purified by the flash column chromatography (40 g, silica gel column), eluted with 0-30% EtOAc in pet ether to get the pure methyl 4-nitro-1-((2-(trimethylsilyl)ethoxy)methyl)-1H-pyrazole-3-carboxylate (3 g, 57% yield) as pale yellow liquid. LC-MS m/z [M+H]⁺=302.17

4-nitro-1-((2-(trimethylsilyl)ethoxy)methyl)-1H-pyrazole-3-carbohydrazide: To a sealed tube containing stirred solution of methyl 4-nitro-1-((2-(trimethylsilyl)ethoxy)methyl)-1H-pyrazole-3-carboxylate (2 g, 6.63 mmol) in ethanol (30 mL) was added hydrazine hydrate (0.66 g, 13.27 mmol) and stirred at 100° C. for 12 h. Progress of the reaction was monitored by TLC and LCMS. After completion of the reaction, concentrated under reduced pressure to get crude product. The crude product was purified by the flash column chromatography (24 g, silica column), eluted with 0-100% EtOAc in pet ether to get 4-nitro-1-((2-(trimethylsilyl)ethoxy)methyl)-1H-pyrazole-3-carbohydrazide (0.9 g, 45% yield) as a colourless liquid. LC-MS m/z [M+H]+=302.17

2-(4-nitro-1-((2-(trimethylsilyl)ethoxy)methyl)-1H-pyrazol-3-yl)-1,3,4-oxadiazole: To a sealed tube containing a stirred solution of 4-nitro-1-((2-(trimethylsilyl)ethoxy)methyl)-1H-pyrazole-3-carbohydrazide (1 g, 3.38 mmol) in acetic acid (2.5 mL) was added triethoxymethane (5 mL) and stirred at 100° C. for 3 h. The progress of the reaction was monitored by TLC. After completion of reaction, the reaction mass was concentrated under reduced pressure to get the crude product. The crude product was purified by the flash column chromatography (24 g, silica column), eluted with 0-80% ethyl acetate in pet-ether to get the 2-(4-nitro-1-((2-(trimethylsilyl)ethoxy)methyl)-1H-pyrazol-3-yl)-1,3,4-oxadiazole (0.75 g, 68% yield) as colourless gummy liquid. LC-MS m/z [M+H]+=312.07.

3-(1,3,4-oxadiazol-2-yl)-1-((2-(trimethylsilyl)ethoxy)methyl)-1H-pyrazol-4-amine: To a stirred solution of 2-(4-nitro-1-((2-(trimethylsilyl)ethoxy)methyl)-1H-pyrazol-3-yl)-1,3,4-oxadiazole (0.8 g, 2.56 mmol) in methanol (8 mL) was added Pd—C (0.32 g) and stirred at room temperature for 6 h under hydrogen bladder pressure. The progress of the reaction was monitored by TLC. After completion of reaction, the reaction mass was diluted with MeOH and filtered on celite pad, the filtrate was concentrated under reduced pressure to get 3-(1,3,4-oxadiazol-2-yl)-1-((2-(trimethylsilyl)ethoxy)methyl)-1H-pyrazol-4-amine (0.4 g, 55% yield) as pale violet liquid. LC-MS m/z [M+H]+=282.13.

As per the method described in Example 2, coupling to Intermediate 1 and deprotection of SEM provided for

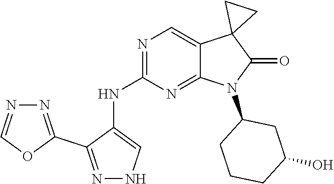 [SMILES: O=C1N([C@@H]2CCC[C@@H](O)C2)c2nc(Nc3c[nH]nc3-c3nnco3)ncc2C12CC2]

2′-((3-(1,3,4-oxadiazol-2-yl)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

### Example 84

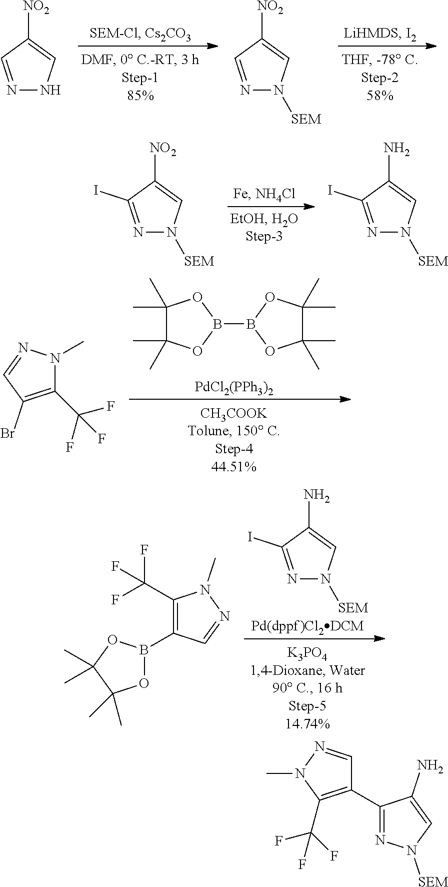

4-nitro-1-((2-(trimethylsilyl)ethoxy)methyl)-1H-pyrazole: To a stirred solution of 4-nitro-1H-pyrazole (5 g, 44.21 mmol) and Cs₂CO₃ (28.7 g, 88.49 mmol) in DMF (50 mL), was added SEM-Cl (11.5 mL, 66.37 mmol) at 0° C. and stirred at room temperature for 3 h. The progress of the reaction was monitored by TLC.

After completion of the reaction, the reaction was quenched with ice-cold water and extracted with ethyl acetate (2×50 mL). The organic layer was washed with ice-cold water (2×10 mL), dried over Na₂SO₄ and concentrated under reduced pressure to give the crude product. The crude compound was purified by Sepa-Bean using silica gel (230-400 mesh), eluting with 0-10% ethyl acetate in pet-ether to give 4-nitro-1-((2-(trimethylsilyl)ethoxy)methyl)-1H-pyrazole as colorless liquid (8 g, 85% yield). ¹H NMR (400 MHz, CDCl₃) δ=8.31 (s, 1H), 8.08-8.14 (m, 1H), 5.45 (s, 2H), 3.59-3.69 (m, 2H), 0.90-1.0 (m, 2H), 0.00-0.05 (m, 9H).

3-iodo-4-nitro-1-((2-(trimethylsilyl)ethoxy)methyl)-1H-pyrazole: To a stirred solution of 4-nitro-1-((2-(trimethylsilyl)ethoxy)methyl)-1H-pyrazole (10 g, 46.94 mmol) in dry THE (100 mL), was added LiHMDS (56 mL) at −78° C. and stirred for 1 h at the same temperature. After 1 h, Iodine (10 g, 78.85 mmol) was added at −78° C. and stirred for 1 h. Progress of the reaction was monitored by TLC. After completion of the reaction, the reaction mass was quenched with sat. NH₄Cl, extracted with ethyl acetate (2×100 mL). The organic layer was dried over Na₂SO₄, concentrated under reduced pressure to give the crude product. The crude compound was purified by Sepa-Bean using silica gel (230-400 mesh), eluting with 0-30% ethyl acetate in pet-ether to give 3-iodo-4-nitro-1-((2-(trimethylsilyl)ethoxy)methyl)-1H-pyrazole as pale yellow liquid (10 g, 58% yield). ¹H NMR (400 MHz, CDCl₃) δ=8.26 (s, 1H), 5.61 (s, 2H), 3.62-3.69 (m, 2H), 0.90-0.99 (m, 2H), 0.00-0.05 (m, 9H).

3-iodo-1-((2-(trimethylsilyl)ethoxy)methyl)-1H-pyrazol-4-amine: To a stirred solution of 3-iodo-4-nitro-1-((2-(trimethylsilyl)ethoxy)methyl)-1H-pyrazole (3 g, 8.13 mmol) in ethanol (30 mL) and water (30 mL), were added NH₄Cl (4.30 g, 81.30 mmol) and iron powder (4.71 g, 81.30 mmol) at room temperature and then stirred at 50° C. for 3 h. The Progress of the reaction was monitored by TLC. After, completion of the reaction, the reaction mass was filtered through plug of celite, the filtrate was concentrated, diluted with ethyl acetate (100 mL) and washed with water (2×50 mL). The organic layer was dried over Na₂SO₄, concentrated under reduced pressure to give the crude product. The crude compound was purified by Sepa-Bean using silica gel (230-400 mesh), eluting with 0-30% ethyl acetate in pet-ether to give 3-iodo-1-((2-(trimethylsilyl)ethoxy)methyl)-1H-pyrazol-4-amine as Pale brown liquid (2.5 g, 91% yield). ¹H NMR (400 MHz, CDCl₃) δ=7.15-7.28 (m, 1H), 5.31-5.40 (m, 2H), 3.49-3.59 (m, 2H), 2.80 (br-s, 2H), 0.88-0.92 (m, 2H), −0.03-0.02 (m, 9H).

1-methyl-4-(4,4,5,5-tetramethyl-1,3,2-dioxaborolan-2-yl)-5-(trifluoromethyl)-1H-pyrazole: To a stirred solution of 4-bromo-1-methyl-5-(trifluoromethyl)-1H-pyrazole (500 mg, 5.155 mmol) and BisPin (720.8 mg, 2.838 mmol) in Toluene (14.40 mL) was added PdCl2(PP3)2 (76.63 mg 0.109 mmol) at 25° C. and stirred for 15 minutes at 150° C. The progress of the reaction was monitored by TLC. After completion of the reaction, the reaction mass was cooled to 25° C. and filtered over celite then rinsed with ethylacetate. The filtrated was concentrated under reduced pressure to give the crude product 1-methyl-4-(4,4,5,5-tetramethyl-1,3,2-dioxaborolan-2-yl)-5-(trifluoromethyl)-1H-pyrazole (720.0 mg, 44.51% yield). LC-MS m/z [M+H]+=277.42

1′-methyl-5′-(trifluoromethyl)-1-((2-(trimethylsilyl)ethoxy)methyl)-1H,1′H-[3,4′-bipyrazol]-4-amine: To a stirred solution of 3-iodo-1-((2-(trimethylsilyl)ethoxy)methyl)-1H-pyrazol-4-amine (500 mg, 1.474 mmol) 1-methyl-4-(4,4,5,5-tetramethyl-1,3,2-dioxaborolan-2-yl)-5-(trifluoromethyl)-1H-pyrazole (488.3 mg, 1.474 mmol) and Potassium phosphate tribasic (782.1 mg, 3.685 mmol) in 1,4-Dioxane (4.5 mL) and Water (0.5 mL) was added Pd(dppf)Cl₂ DCM complex (120.4 mg, 0.147 mmol at 25° C. and then stirred at 90° C. for 16 h. Progress of the reaction was monitored by TLC. After, completion of the reaction, the reaction mass was diluted with water and extracted with ethyl acetate (2×15 mL). The organic layer was washed with ice-cold water (2×10 mL), dried over Na₂SO₄ and concentrated under reduced pressure to give the crude product. The crude compound was purified by Sepa-Bean using silica gel (230-400 mesh), eluting with 50-70% ethyl acetate in pet-ether to give 1′-methyl-5′-(trifluoromethyl)-1-((2-(trimethylsilyl)ethoxy)methyl)-1H,1′H-[3,4′-bipyrazol]-4-amine as white color solid (250 mg, 14.74% yield). LC-MS m/z [M+H]+=362.50

As per the method described in Example 2, coupling to Intermediate 1 and deprotection of SEM provided for

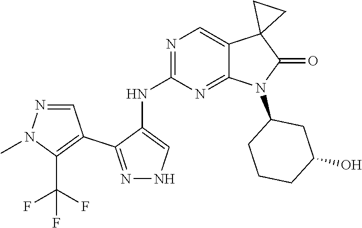 [SMILES: Cn1ncc(-c2n[nH]cc2Nc2ncc3c(n2)N([C@@H]2CCC[C@@H](O)C2)C(=O)C32CC2)c1C(F)(F)F]

7′-((1R,3R)-3-hydroxycyclohexyl)-2′-((1′-methyl-5′-(trifluoromethyl)-1H,1′H-[3,4′-bipyrazol]-4-yl)amino)spiro[cyclopropane-1,5′-pyrrolo[2,3-d]pyrimidin]-6′(7′H)-one

### Example 85

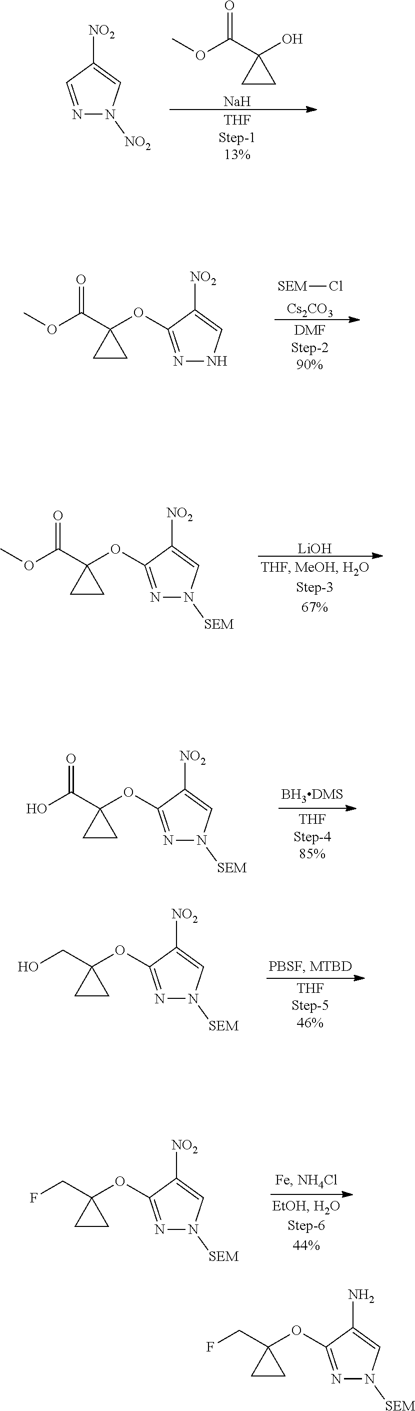

methyl 1-((4-nitro-1H-pyrazol-3-yl)oxy)cyclopropane-1-carboxylate: To a stirred solution of methyl 1-hydroxycyclopropane-1-carboxylate (3 g, 25.84 mmol) in THF (30 mL) was added sodium hydride (1.033 g, 25.84 mmol) and stirred for 30 minutes at 0° C., this reaction mixture was added to a stirred solution of 1,4-dinitro-1H-pyrazole (4.1 g, 25.84 mmol) in THF (120 mL) at −78° C. The resulting reaction mixture was stirred at same temperature foe 1 hour. The reaction progress was monitored by LCMS & TLC. LC-MS showed desired product formation. The reaction mixture was quenched with sat. NH₄Cl solution and extracted with ethyl acetate, combined the organic layers and dried over sodium sulphate filtered and concentrated under reduced vacuum pressure to give crude product. The crude product was purified by the SepaBean column chromatography and eluted with 20-35% EtOAc in pet ether. All pure fractions were collected and concentrated to get the methyl 1-((4-nitro-1H-pyrazol-3-yl)oxy)cyclopropane-1-carboxylate (1 g, 13% yield) as off white solid. LC-MS m/z [M+H]+=228.28 methyl 1-((4-nitro-1-((2-(trimethylsilyl)ethoxy)methyl)-1H-pyrazol-3-yl)oxy)cyclopropane-1-carboxylate: To a stirred solution of methyl 1-((4-nitro-1H-pyrazol-3-yl)oxy)cyclopropane-1-carboxylate (900 mg, 3.96 mmol) was taken in DMF (9 mL) was added cesium carbonate (2.6 g, 7.92 mmol) and SEM-Cl (991 mg, 1 mL, 5.94 mmol) dropwise at 0° C. The resulting reaction mixture was stirred for 2 hours at 27° C., The reaction progress was monitored by LC-MS & TLC. LC-MS showed desired product formation. The reaction mixture was quenched with water and extracted with ethyl acetate, combined the organic layers and dried over sodium sulphate filtered and concentrated under reduced vacuum pressure to give crude product. The crude product was purified by the SepaBean column chromatography and eluted with 10-20% EtOAc in pet ether. All pure fractions were collected and concentrated to get the pure compound of methyl 1-((4-nitro-1-((2-(trimethylsilyl)ethoxy)methyl)-1H-pyrazol-3-yl)oxy)cyclopropane-1-carboxylate (1.3 g, 90% yield) as a pale yellow oil. LC-MS m/z [M+H]+=300.12

methyl 1-((4-nitro-1-((2-(trimethylsilyl)ethoxy)methyl)-1H-pyrazol-3-yl)oxy)cyclopropane-1-carboxylate: To a stirred solution of methyl 1-((4-nitro-1-((2-(trimethylsilyl)ethoxy)methyl)-1H-pyrazol-3-yl)oxy)cyclopropane-1-carboxylate (1.1 g, 3.077 mmol) was taken in methanol (11 mL), THE (11 mL) and water (6 mL) was added LiOH (0.22 g, 9.232 mmol). Then stirred the reaction mixture for 3 hour at 27° C., Reaction progress was monitored by LC-MS. LC-MS showed desired product formation. The reaction mixture was concentrated under reduced vacuum pressure to give crude product and acidified with 1N HCl solution and extracted with ethyl acetate, combined the organic layers and dried over sodium sulphate filtered and concentrated under reduced vacuum pressure to give 1-((4-nitro-1-((2-(trimethylsilyl)ethoxy)methyl)-1H-pyrazol-3-yl)oxy)cyclopropane-1-carboxylic acid (910 mg, 67% yield) as pale yellow gummy liquid. LC-MS m/z [M+H]+=343.45

(1-((4-nitro-1-((2-(trimethylsilyl)ethoxy)methyl)-1H-pyrazol-3-yl)oxy)cyclopropyl)methanol: To a stirred solution of 1-((4-nitro-1-((2-(trimethylsilyl)ethoxy)methyl)-1H-pyrazol-3-yl)oxy)cyclopropane-1-carboxylic acid (900.0 mg, 2.621 mmol) was taken in THE (15 mL) was added BH3·DMS (2.6 mL, 2M, 5.242 mmol) dropwise at 0° C. The resulting reaction mixture was stirred for 3 hours at 65° C. Reaction progress was monitored by LCMS. The reaction mixture was quenched with methanol & concentrated under reduced vacuum pressure to give crude product. The crude product was purified by the Sepabean column chromatography and eluted with 40-70% EtOAc in pet ether. All pure fractions were collected and concentrated to get (1-((4-nitro-1-((2-(trimethylsilyl)ethoxy)methyl)-1H-pyrazol-3-yl)oxy)cyclopropyl)methanol (750 mg, 85% yield) as an off white solid. LC-MS m/z [M+H]+=330.18

3-(1-(fluoromethyl)cyclopropoxy)-4-nitro-1-((2-(trimethylsilyl)ethoxy)methyl)-1H-pyrazole: To a stirred solution of (1-((4-nitro-1-((2-(trimethylsilyl)ethoxy)methyl)-1H-pyrazol-3-yl)oxy)cyclopropyl)methanol (300 mg, 910.66 μmol) was taken in THE (9 mL) was added PBSF (0.24 mL, 1.366 mmol) in THE (3 mL) dropwise at 0° C. & MTBD (210 mg, 1.3660 mmol), Then stirred the reaction mixture for 3 hour at 0° C., Reaction progress was monitored by LC-MS. The reaction mixture was quenched with water and extracted with DCM, combined the organic layers and dried over sodium sulphate filtered and concentrated under reduced vacuum pressure to give crude product. The crude product was purified by the Sepabean column chromatography and eluted with 10-30% EtOAc in pet ether. All pure fractions were collected and concentrated to get 3-(1-(fluoromethyl)cyclopropoxy)-4-nitro-1-((2-(trimethylsilyl)ethoxy)methyl)-1H-pyrazole (140 mg, 46% yield) as a white solid. LC-MS m/z [M+H]+=332.48

3-(1-(fluoromethyl)cyclopropoxy)-1-((2-(trimethylsilyl)ethoxy)methyl)-1H-pyrazol-4-amine: To a stirred solution of 3-(1-(fluoromethyl)cyclopropoxy)-4-nitro-1-((2-(trimethylsilyl)ethoxy)methyl)-1H-pyrazole (140 mg, 422.42 μmol) was taken in ethanol (1.4 mL) and water (1.4 mL) was added iron (118 mg, 2.11 mmol) and ammonium chloride (113 mg, 2.1121 mmol). The resulting reaction mixture was stirred for 2 hours at 50° C. The reaction progress was monitored by LCMS & TLC. The reaction mixture was filtered through celite pad & washed with 10% MeoH in DCM, dried over sodium sulphate, filtered and concentrated under reduced vacuum pressure to give 3-(1-(fluoromethyl)cyclopropoxy)-1-((2-(trimethylsilyl)ethoxy)methyl)-1H-pyrazol-4-amine (120 mg, 44% yield) as pale orange oil. LC-MS m/z [M+H]+=302.23

As per the method described in Example 2, coupling to Intermediate 1 and deprotection of SEM provided for

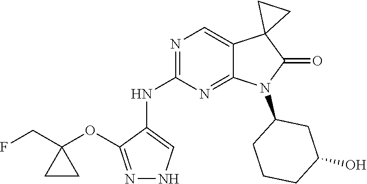 [SMILES: O=C1N([C@@H]2CCC[C@@H](O)C2)c2nc(Nc3c[nH]nc3OC3(CF)CC3)ncc2C12CC2]

2′-((3-(1-(fluoromethyl)cyclopropoxy)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one

| Structure | Data |
|---|---|
| 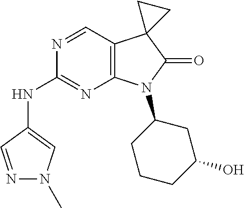 [SMILES: Cn1cc(Nc2ncc3c(n2)N([C@@H]2CCC[C@@H](O)C2)C(=O)C32CC2)cn1] | LC-MS m/z [M + H]+ = 355.30, ¹H NMR (400 MHz, DMSO- d₆) δ = 9.35 (s, 1H), 7.87 (s, 1H), 7.85 (s, 1H), 7.48 (s, 1H), 4.55-4.80 (m, 2H), 4.13 (s, 1H), 3.79 (s, 3H), 2.40-2.50 (m, 1H), 2.20-2.35 (m, 1H), 1.51-1.85 (m, 7H), 1.35-1.50 (m, 3H). |
| (Example 1) | |
| 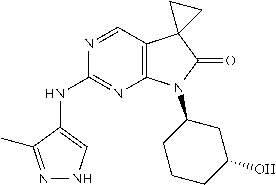 [SMILES: Cc1n[nH]cc1Nc1ncc2c(n1)N([C@@H]1CCC[C@@H](O)C1)C(=O)C21CC1] | LC-MS m/z [M + H]+ = 355.27, ¹H NMR (400 MHz, DMSO- d₆) δ = 12.17 (br-s, 1H), 8.57 (br-s, 1H), 7.81 (s, 1H), 7.68 (s, 1H), 4.60-4.75 (m, 1H), 4.54 (s, 1H), 4.08 (s, 1H), 3.17 (d, J = 5.2 Hz, 2H), 2.40-2.50 (m, 1H), 2.10-2.30 (m, 4H), 1.50-1.82 (m, 7H), 1.30-1.50 (m, 3H). |
| (Example 2) | |
| 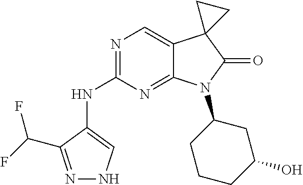 [SMILES: O=C1N([C@@H]2CCC[C@@H](O)C2)c2nc(Nc3c[nH]nc3C(F)F)ncc2C12CC2] | LC-MS m/z [M + H]+ = 391.34, ¹H NMR (400 MHz, DMSO- d₆) δ = 13.11 (s, 1H), 8.82 (s, 1H), 8.04 (s, 1H), 7.87 (s, 1H), 7.17 (t, J = 54 Hz, 1H), 4.62-4.75 (m, 1H), 4.56 (d, J = 2.8 Hz, 1H), 4.09 (s, 1H), 2.42-2.50 (m, 1H), 2.15-2.28 (m, 1H), 1.50-1.82 (m, 7H), 1.45-1.50 (m, 2H), 1.38 (t, J = 12.8 Hz, 1H). |
| (Example 3) | |
| 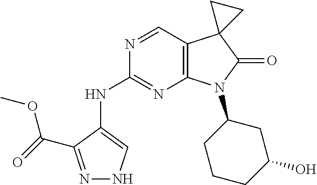 [SMILES: COC(=O)c1n[nH]cc1Nc1ncc2c(n1)N([C@@H]1CCC[C@@H](O)C1)C(=O)C21CC1] | LC-MS m/z [M + H]+ = 399.29, ¹H NMR (400 MHz, DMSO- d₆) δ = 13.54 (br-s, 1H), 8.57 (br-s, 1H), 8.27 (s, 1H), 7.98 (s, 1H), 4.65-4.78 (m, 1H), 4.65 (d, J = 2.4 Hz, 1H). 4.13 (s, 1H), 3.88 (s, 3H), 2.42-2.52 (m, 1H), 2.20-2.35 (m, 1H), 1.55-1.82 (m, 7H), 1.38-1.55 (m, 3H). |
| (Example 4) | |
| 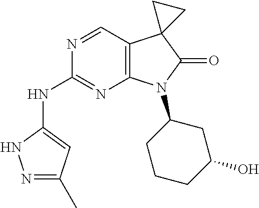 [SMILES: Cc1cc(Nc2ncc3c(n2)N([C@@H]2CCC[C@@H](O)C2)C(=O)C32CC2)[nH]n1] | LC-MS m/z [M + H]+ = 355.30, ¹H NMR (400 MHz, DMSO- d₆) δ = 11.79 (br-s, 1H), 9.45 (br-s, 1H), 7.86 (s, 1H), 6.34 (br-s, 1H), 4.63-4.73 (m, 1H), 4.57 (s, 1H), 4.11 (s, 1H), 2.50-2.60 (m, 1H), 2.25-2.38 (m, 1H), 2.19 (s, 3H), 1.51- 1.85 (m, 7H), 1.35-1.50 (m, 3H). |
| (Example 5) | |
| 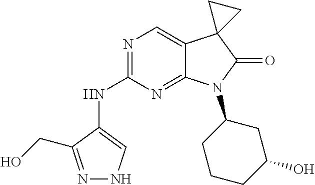 [SMILES: O=C1N([C@@H]2CCC[C@@H](O)C2)c2nc(Nc3c[nH]nc3CO)ncc2C12CC2] | LC-MS m/z [M + H]+ = 371.30, ¹H NMR (400 MHz, DMSO- d₆) δ = 12.34 (s, 1H), 8.51 (s, 1H), 7.85 (s, 2H), 5.12 (br-s, 1H), 4.45-4.75 (m, 4H), 4.10 (s, 1H), 2.52-2.62 (m, 1H), 2.15-2.30 (m, 1H), 1.51-1.85 (m, 7H), 1.32-1.50 (m, 3H). |
| (Example 6) | |
| 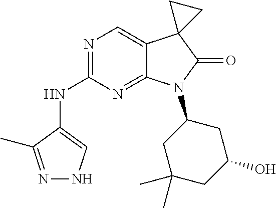 [SMILES: Cc1n[nH]cc1Nc1ncc2c(n1)N([C@H]1C[C@H](O)CC(C)(C)C1)C(=O)C21CC1] | LC-MS m/z [M + H]+ = 383.43, ¹H NMR (400 MHz, DMSO- d₆) δ = 12.15 (br-s, 1H), 8.55 (s, 1H), 7.82 (s, 1H), 7.62 (s, 1H), 4.82 (t, J = 12.4 Hz, 1H), 4.54 (s, 1H), 4.10 (s, 1H), 2.38-2.48 (m, 1H), 2.10-2.35 (m, 4H), 1.40-1.70 (m, 6H), 1.20-1.38 (m, 2H), 1.23 (s, 3H), 0.85 (s, 3H). |
| (Example 7) | |
| 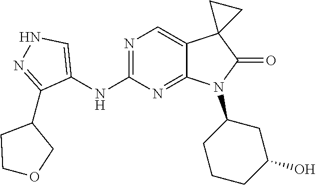 [SMILES: O=C1N([C@@H]2CCC[C@@H](O)C2)c2nc(Nc3c[nH]nc3C3CCOC3)ncc2C12CC2] | LC-MS m/z: [M + H]⁺ = 411.19 (99%) ¹H NMR (400 MHz, DMSO-d₆) δ = 12.459-12.353 (m, 1H), 8.600 (br s, 2H), 7.802 (m, 1H), 7.216-6.90 (m, 1H), 4.77-4.38 (m, 2H), 4.20-4.01 (m, 1H), 3.95 (t, J = 7.6 Hz, 1H), 3.85 (br d, J = 5.6 Hz, 1H), 3.78-3.68 (m,1H), 3.62 (br s, 1H), 3.56-3.46 (m, 1H), 2.45-2.30 (m, 1H), 2.25-2.10 (m, 3H), 2.09- 1.97 (m, 1H), 1.81-1.56 (m, 9H). |
| (Example 8) | |
| 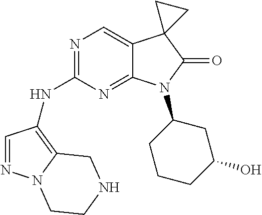 [SMILES: O=C1N([C@@H]2CCC[C@@H](O)C2)c2nc(Nc3cnn4c3CNCC4)ncc2C12CC2] | LC-MS m/z: [M + H]⁺ =396.22 (96.27%) ¹H NMR (400 MHz, DMSO-d₆) δ = 8.707 (s, 1H), 7.80 (s, 1H), 7.53 (s, 1H), 4.75-4.58 (m, 2H), 4.12-4.03 (m, 1H), 3.92 (t, J = 5.4 Hz, 2H), 3.85-3.76 (m, 2H), 3.13-3.01 (m, 2H), 2.46-2.30 (m, 1H), 2.29-2.13 (m, 1H), 1.763-1.516 (m, 10H). |
| (Example 9) | |
| 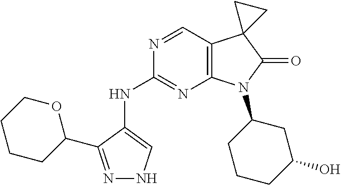 [SMILES: O=C1N([C@@H]2CCC[C@@H](O)C2)c2nc(Nc3c[nH]nc3C3CCCCO3)ncc2C12CC2] | LC-MS m/z: [M + H]⁺ = 425.22 (97%); ¹H NMR (400 MHz, DMSO-d₆) δ = 12.52-12.43 (m, 1H), 8.53-8.47 (m, 1H), 8.16-7.99 (m, 1H), 7.87-7.65 (m, 1H), 4.82-4.45 (m, 3H), 4.31-3.99 (m, 2H), 3.68-3.45 (m, 1H), 2.74-2.61 (m, 1H), 2.36-2.05 (m, 1H), 1.92-1.18 (m, 16H). |
| (Example 10) | |
| 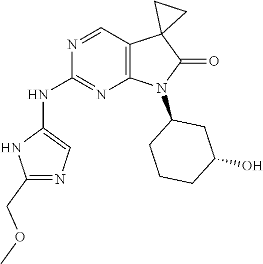 [SMILES: COCc1ncc(Nc2ncc3c(n2)N([C@@H]2CCC[C@@H](O)C2)C(=O)C32CC2)[nH]1] | LC-MS m/z [M + H]+ = 385.32 (99%) ¹H NMR (400 MHz, DMSO-d₆) δ = 11.97-11.78 (m, 1H), 9.37 (s, 1H), 7.88 (s, 1H), 7.19 (s, 1H), 4.73-4.67 (m, 1H), 4.57-4.54 (m, 1H), 4.32 (s, 2H), 4.18-4.00 (m, 1H), 3.27(m, 3H), 2.71-2.53 (m, 1H), 2.40-2.24 (m, 1H), 1.87-1.36 (m, 10H). |
| (Example 11) | |
| 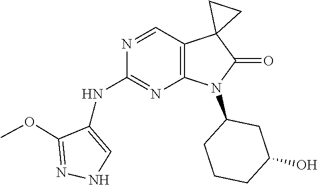 [SMILES: COc1n[nH]cc1Nc1ncc2c(n1)N([C@@H]1CCC[C@@H](O)C1)C(=O)C21CC1] | LC-MS m/z: [M + H]⁺ = 371.38 (98%) ¹H NMR (400 MHz, DMSO-d₆) δ = 11.71-11.51 (s, 1H), 8.19 (s, 1H), 7.78 (s, 1H), 7.647-7.643 (d, 1H), 4.649-4.619 (m, 1H), 4.52 (d, J = 2.8 Hz, 1H), 4.06 (br s, 1H), 3.78 (s, 3H), 2.35-2.10 (m, 1H), 1.83-1.19 (m, 10H). |
| (Example 12) | |
| 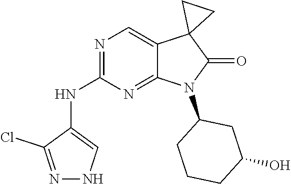 [SMILES: O=C1N([C@@H]2CCC[C@@H](O)C2)c2nc(Nc3c[nH]nc3Cl)ncc2C12CC2] | LC-MS m/z: [M + H]⁺ = 375.27 (98%) ¹H NMR (400 MHz, DMSO-d₆, 298 K) δ (ppm) = 13.02-12.52 (m, 1H), 8.62- 8.43 (m, 2H), 7.98-7.76 (m, 2H), 4.74-4.43 (m, 2H), 4.07 (br s, 1H), 2.49-2.52 (m, 1H), 2.35-1.99 (m, 1H), 1.84- 1.14 (m, 11H). |
| (Example 13) | |
| 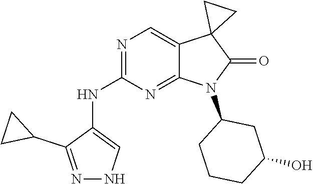 [SMILES: O=C1N([C@@H]2CCC[C@@H](O)C2)c2nc(Nc3c[nH]nc3C3CC3)ncc2C12CC2] | LC-MS m/z: [M + H]⁺ = 381.35 (98%) , ¹H NMR (400 MHz, DMSO-d6, 298 K) δ (ppm) = 12.22-11.99 (m, 1H), 8.71- 8.38 (m, 1H), 7.93-7.43 (m, 2H), 4.74-4.44 (m, 2H), 4.13- 3.91 (m, 1H), 2.50-1.84 (m, 3H), 1.84-1.16 (m, 12H), 0.86-0.60 (m, 4H) |
| (Example 14) | |
| 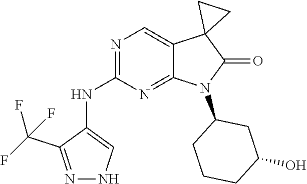 [SMILES: O=C1N([C@@H]2CCC[C@@H](O)C2)c2nc(Nc3c[nH]nc3C(F)(F)F)ncc2C12CC2] | LC-MS m/z: [M + H]⁺ = 409.30 (95%), ¹H NMR (400 MHz, DMSO-d6: 298 K) δ (ppm) = 13.479 (s, 1H), 8.547 (s, 1H), 8.024 (s, 1H), 7.816 (s, 1H), 4.631-4.525 (m, 2H), 4.052 (br s, 1H), 2.457-2.392 (m, 1H), 2.159-2.136 (m, 1H), 1.77-1.11 (m, 10H). |
| (Example 15) | |
| 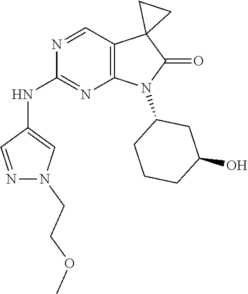 [SMILES: COCCn1cc(Nc2ncc3c(n2)N([C@H]2CCC[C@H](O)C2)C(=O)C32CC2)cn1] | LC-MS m/z [M + H]+ = 399.34, ¹H NMR (400 MHz, DMSO- d₆) δ = 9.36 (s, 1H), 7.88-7.90 (m, 2H), 7.52 (s, 1H), 4.71 (t, J = 12.4 Hz, 1H), 4.59 (s, 1H), 4.18 (t, J = 5.6 Hz, 2H), 4.12 (s, 1H), 3.65 (t, J = 5.6 Hz, 2H), 3.22 (s, 3H), 2.45- 2.50 (m, 1H), 2.20-2.35 (m, 1H), 1.52-1.85 (m, 7H), 1.38- 1.50 (m, 3H). |
| (Example 16) | |
| 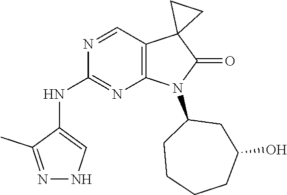 [SMILES: Cc1n[nH]cc1Nc1ncc2c(n1)N([C@@H]1CCCC[C@@H](O)C1)C(=O)C21CC1] | LC-MS m/z [M + H]⁺ = 369.40, ¹H NMR (400 MHz, DMSO-d₆) δ = 12.09 (br-s, 1H), 8.54 (s, 1H), 7.80 (s, 1H), 7.69 (s, 1H), 4.68-4.78 (m, 1H), 4.49 (s, 1H), 3.98 (s, 1H), 2.50-2.60 (m, 1H), 2.10-2.25 (m, 4H), 1.63--1.82 (m, 5H), 1.56-1.62 (m, 3H), 1.41-1.55 (m, 3H), 1.30-1.50 (m, 1H). |
| (Example 17) | |
| 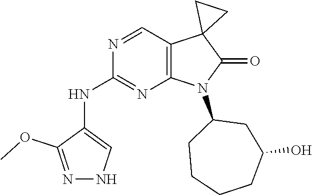 [SMILES: COc1n[nH]cc1Nc1ncc2c(n1)N([C@@H]1CCCC[C@@H](O)C1)C(=O)C21CC1] | LC-MS m/z [M + H]+ = 385.37, ¹H NMR (400 MHz, DMSO- d₆) δ = 11.63 (s, 1H), 8.16 (s, 1H), 7.78 (s, 1H), 7.67 (s, 1H), 4.64-4.73 (m, 1H), 4.47 (s, 1H), 3.97 (s, 1H), 3.79 (s, 3H), 2.50-2.60 (m, 1H), 2.12-2.28 (m, 1H), 1.52-1.80 (m, 8H), 1.52-1.30 (m, 4H). |
| (Example 18) | |
| 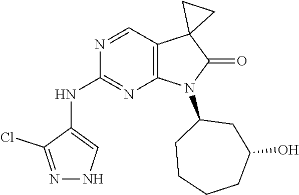 [SMILES: O=C1N([C@@H]2CCCC[C@@H](O)C2)c2nc(Nc3c[nH]nc3Cl)ncc2C12CC2] | LC-MS m/z [M + H]+ = 389.13, ¹H NMR (400 MHz, DMSO- d₆) δ = 12.86 (s, 1H), 8.53 (s, 1H), 7.92 (s, 1H), 7.82 (s, 1H), 4.68-4.75 (m, 1H), 4.48 (d, J = 3.2 Hz, 1H), 3.97 (s, 1H), 2.50-2.60 (m, 1H), 2.12-2.25 (m, 1H), 1.55-1.80 (m, 8H), 1.42-1.54 (m, 3H), 1.28-1.40 (m, 1H). |
| (Example 19) | |
| 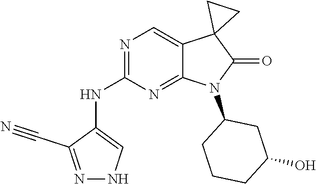 [SMILES: N#Cc1n[nH]cc1Nc1ncc2c(n1)N([C@@H]1CCC[C@@H](O)C1)C(=O)C21CC1] | LC-MS m/z [M + H]⁺ = 366.24, ¹H NMR (400 MHz, DMSO-d₆) δ = 13.71 (br-s, 1H), 9.34 (s, 1H), 8.05 (s, 1H), 7.89 (s, 1H), 4.62-4.72 (m, 1H), 4.55 (d, J = 2.4 Hz, 1H), 4.08 (s, 1H), 2.42-2.52 (m, 1H), 2.12-2.26 (m, 1H), 1.30-1.82 (m, 10H). |
| (Example 20) | |
| 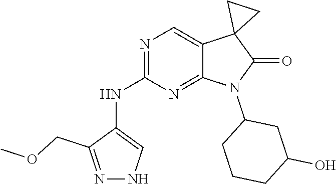 [SMILES: COCc1n[nH]cc1Nc1ncc2c(n1)N(C1CCCC(O)C1)C(=O)C21CC1] | LC-MS m/z [M + H]⁺ = 385.49, ¹H NMR (400 MHz, DMSO-d₆) δ = 12.55 (br-s, 1H), 8.58 (br-s, 1H), 7.82-7.94 (m, 2H), 4.63- 4.73(m, 1H), 4.56 (d, J = 2.4 Hz, 1H), 4.48 (s, 2H), 4.09 (s, 1H), 3.22 (s, 3H), 2.45-2.55 (m, 1H), 2.15-2.28 (m, 1H), 1.51-1.84 (m, 7H), 1.32-1.50 (m, 3H). |
| (Example 21) | |
| 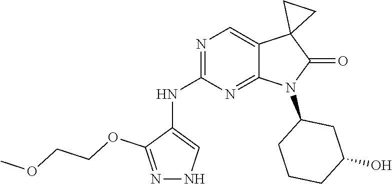 [SMILES: COCCOc1n[nH]cc1Nc1ncc2c(n1)N([C@@H]1CCC[C@@H](O)C1)C(=O)C21CC1] | LC-MS m/z: [M + H]⁺ = 415.20 (99%) ¹H NMR (400 MHz, DMSO-d₆) δ = 11.673 (br s, 1H), 8.117 (s, 1H), 7.795 (s, 1H), 7.665 (s, 1H), 4.658-4.598 (m, 1H), 4.530 (brs, 1H), 4.220- 4.196 (m, 2H), 4.066 (br s, 1H), 3.629-3.605 (m, 2H), 3.318 (s, 3H), 2.510 (s, 1H), 2.328-2.179 (m, 1H), 1.756-1.513 (m, 9H), 1.480-1.337 (m, 1H). |
| (Example 22) | |
| 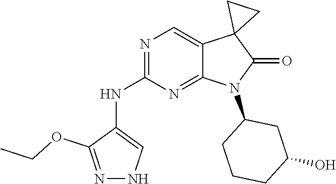 [SMILES: CCOc1n[nH]cc1Nc1ncc2c(n1)N([C@@H]1CCC[C@@H](O)C1)C(=O)C21CC1] | LC-MS m/z: [M + H]⁺ = 385.33 (97%) ¹H NMR (400 MHz, DMSO-d₆) δ = 11.615 (brs, 1H), 8.105 (s, 1H), 7.788 (s, 1H), 7.638 (s, 1H), 4.6.52-4.591 (m, 1H), 4.533 (brs, 1H), 4.174- 4.121 (m, 2H), 4.06 (br s, 1H), 2.497 (s, 1H), 2.235-2.209(m, 1H), 1.755-1.462 (m, 9H), 1.454-1.281 (m, 4H). |
| (Example 23) | |
| 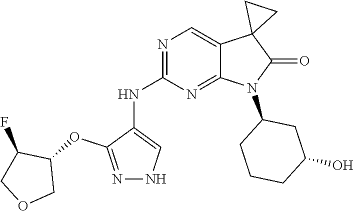 [SMILES: O=C1N([C@@H]2CCC[C@@H](O)C2)c2nc(Nc3c[nH]nc3O[C@@H]3COC[C@H]3F)ncc2C12CC2] | LC-MS m/z [M + H]⁺ = 445.28, Chiral HPLC: 98.61% (Rt = 7.70), ¹H NMR (400 MHz, DMSO-d₆) δ = 11.85 (s, 1H), 8.32 (br-s, 1H), 7.808 (s, 1H), 7.733-7.737 (m, 1H), 5.26-5.39 (m, 1H), 5.07-5.12 (m, 1H), 4.54-4.67 (m, 1H), 4.542-4.549 (d, J = 2.8 Hz, 1H), 4.10-4.14 (m, 1H), 4.07-4.10 (m, 1H), 3.81- 3.97 (m, 3H), 2.40-2.45 (m, 1H), 2.15-2.25 (m, 1H), 1.30- 1.75 (m, 10H). |
| (Example 24) | |
| 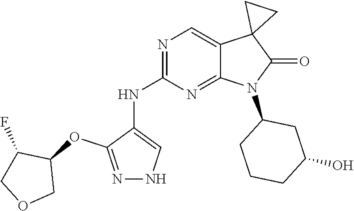 [SMILES: O=C1N([C@@H]2CCC[C@@H](O)C2)c2nc(Nc3c[nH]nc3O[C@H]3COC[C@@H]3F)ncc2C12CC2] | LC-MS m/z [M + H]⁺ = 445.28, Chiral HPLC: 96.58% (Rt = 9.32), ¹H NMR (400 MHz, DMSO-d₆) δ = 11.85 (s, 1H), 8.32 (br-s, 1H), 7.808 (s, 1H), 7.733-7.737 (m, 1H), 5.26-5.39 (m, 1H), 5.07-5.12 (m, 1H), 4.54-4.67 (m, 1H), 4.542-4.549 (d, J = 2.8 Hz, 1H), 4.10-4.14 (m, 1H), 4.07-4.10 (m, 1H), 3.81- 3.97 (m, 3H), 2.40-2.45 (m, 1H), 2.15-2.25 (m, 1H), 1.30- 1.75 (m, 10H). |
| (Example 25) | |
| 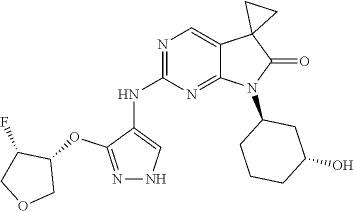 [SMILES: O=C1N([C@@H]2CCC[C@@H](O)C2)c2nc(Nc3c[nH]nc3O[C@@H]3COC[C@@H]3F)ncc2C12CC2] | LC-MS m/z [M + H]⁺ = 445.28, Chiral HPLC: 99.98% (Rt = 3.97), ¹H NMR (400 MHz, DMSO-d₆) δ = 11.803 (s, 1H), 8.191 (s, 1H), 7.802 (s, 1H), 7.707 (s, 1H), 5.29-5.43 (m, 1H), 5.02-5.08 (m, 1H), 4.58-4.70 (m, 1H), 4.533-4.539 (d, J = 2.4 Hz, 1H), 4.05-4.11 (m, 1H), 4.011-4.017 (m, 1H), 3.91-3.95 (m, 1H), 3.70-3.74 (m, 1H), 2.41-2.45 (m, 1H), 2.16-2.25 (m, 1H), 1.23-1.80 (m, 10H). |
| (Example 26) | |
| 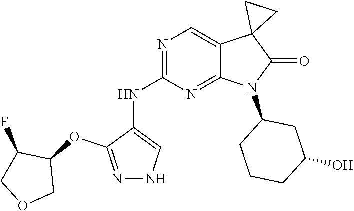 [SMILES: O=C1N([C@@H]2CCC[C@@H](O)C2)c2nc(Nc3c[nH]nc3O[C@H]3COC[C@H]3F)ncc2C12CC2] | LC-MS m/z [M + H]⁺ = 445.24, Chiral HPLC: 99.52% (Rt = 5.14), ¹HNMR (400 MHz, DMSO-d₆) δ = 11.803 (s, 1H), 8.191 (s, 1H), 7.802 (s, 1H), 7.707 (s, 1H), 5.29-5.43 (m, 1H), 5.02-5.08 (m, 1H), 4.58-4.70 (m, 1H), 4.533-4.539 (d, J = 2.4 Hz, 1H), 4.05-4.11 (m, 1H), 4.011-4.017 (m, 1H), 3.91-3.95 (m, 1H), 3.70-3.74 (m, 1H), 2.41-2.45 (m, 1H), 2.16-2.25 (m, 1H), 1.23-1.80 (m, 10H). |
| (Example 27) | |
| 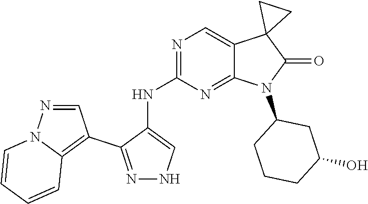 [SMILES: O=C1N([C@@H]2CCC[C@@H](O)C2)c2nc(Nc3c[nH]nc3-c3cnn4ccccc34)ncc2C12CC2] | LC-MS m/z [M + H]⁺ = 457.32, ¹H NMR (400 MHz, DMSO-d₆) δ = 12.71-12.88 (br-s, 1H, rotamers), 8.59-8.65 (m, 2H), 8.08-8.20 (m, 2H), 7.63-7.80 (m, 2H), 7.26-7.29 (m, 1H), 6.90-6.93 (m, 1H), 4.43-4.60 (m, 2H), 3.70-4.15 (m, 1H), 1.85-2.30 (m, 2H), 1.15-1.75 (m, 10H). |
| (Example 28) | |
| 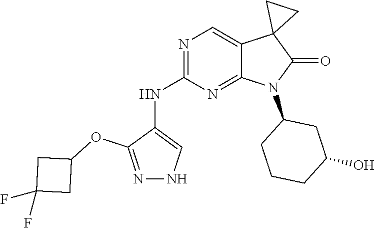 [SMILES: O=C1N([C@@H]2CCC[C@@H](O)C2)c2nc(Nc3c[nH]nc3OC3CC(F)(F)C3)ncc2C12CC2] | LC-MS m/z [M + H]⁺ = 447.26, ¹H NMR (400 MHz, DMSO-d₆) δ = ¹H NMR (400 MHz, DMSO-d₆) δ = 11.78 (s, 1H), 8.27 (br-s, 1H), 7.802 (s, 1H), 7.69-7.70 (m, 1H), 4.78-4.90 (m, 1H), 4.58-4.70 (m, 1H), 4.535-4.542 (d, J=2.8 Hz, 1H), 4.05- 4.11 (m, 1H), 3.00-3.107 (m, 2H), 2.65-2.80 (m, 2H), 2.40- 2.45 (m, 1H), 2.15-2.28 (m, 1H), 1.30-1.85 (m, 10H). |
| (Example 29) | |
| 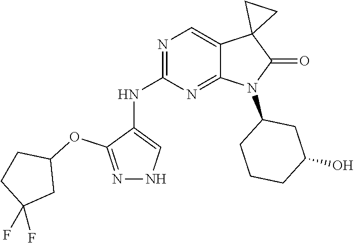 [SMILES: O=C1N([C@@H]2CCC[C@@H](O)C2)c2nc(Nc3c[nH]nc3OC3CCC(F)(F)C3)ncc2C12CC2] | LC-MS m/z [M + H]⁺ = 461.29, ¹H NMR (400 MHz, DMSO- d₆) δ = 11.72 (s, 1H), 8.19 (br-s, 1H), 7.805 (br-s, 1H), 7.702- 7.706 (m, 1H), 5.0-5.08 (m, 1H), 4.60-4.70 (m, 1H), 4.537- 4.544 (d, J = 2.8 Hz, 1H), 4.05-4.10 (m, 1H), 2.55-2.61 (m, 2H), 2.025-2.35 (m, 6H), 1.45-1.80 (m, 9H), 1.28-1.41 (m, 1H). |
| (Example 30) | |
| 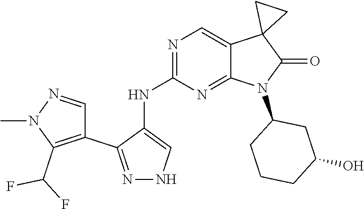 [SMILES: Cn1ncc(-c2n[nH]cc2Nc2ncc3c(n2)N([C@@H]2CCC[C@@H](O)C2)C(=O)C32CC2)c1C(F)F] | LC-MS m/z [M + H]⁺ = 471.26, ¹H NMR (400 MHz, DMSO-d₆) δ = 12.89 (s, 1H), 8.49 (br-s, 1H), 7.76-7.83 (m, 2H), 7.42- 7.69 (m, 2H), 4.50-4.57 (m, 2H), 3.95-4.05 (m, 1H), 3.96 (s, 3H), 2.31-2.35 (m, 1H), 1.98-2.15 (m, 1H), 1.41-1.75 (m, 9H), 1.15-1.35 (m, 1H). |
| (Example 31) | |
| 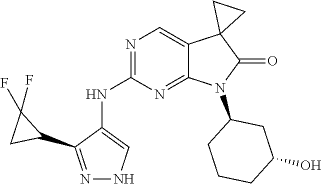 | LC-MS m/z [M + H]⁺ = 417.23, Chiral HPLC: 99.81% (Rt: 2.46 min), ¹H NMR (400 MHz, DMSO-d₆) δ = 11.47 (s, 1H), 8.87 (br-s, 1H), 7.60-7.89 (m, 2H), 4.65-4.70 (m, 1H), 4.552- 4.558 (d, J = 2.4 Hz, 1H), 4.05-4.15 (m, 1H), 3.03-3.108 (m, 1H), 2.451-2.456 (m, 1H), 2.15-2.25 (m, 1H), 1.85-2.01 (m, 2H), 1.51-1.85 (m, 7H), 1.45-1.50 (m, 2H), 1.31-1.41 (m, 1H). |
| (Example 32) | |
| 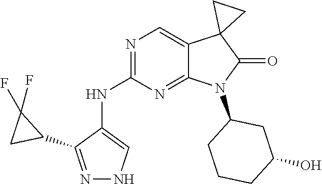 | LC-MS m/z [M + H]⁺ = 417.23, Chiral HPLC: 99.01% (Rt: 4.21 min), ¹H NMR (400 MHz, DMSO-d₆) δ = 11.47 (s, 1H), 8.87 (br-s, 1H), 7.60-7.89 (m, 2H), 4.65-4.70 (m, 1H), 4.552- 4.558 (d, J = 2.4 Hz, 1H), 4.05-4.15 (m, 1H), 3.03-3.108 (m, 1H), 2.451-2.456 (m, 1H), 2.15-2.25 (m, 1H), 1.85-2.01 (m, 2H), 1.51-1.85 (m, 7H), 1.45-1.50 (m, 2H), 1.31-1.41 (m, 1H). |
| (Example 33) | |
| 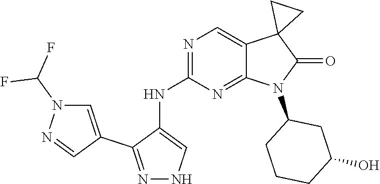 [SMILES: O=C1N([C@@H]2CCC[C@@H](O)C2)c2nc(Nc3c[nH]nc3-c3cnn(C(F)F)c3)ncc2C12CC2] | LC-MS m/z [M + H]⁺ = 457.23. ¹H NMR (400 MHz, DMSO- d₆) δ = 12.78-12.84 (br-s, 1H, rotamers), 8.66 (br-s, 1H), 8.36 (s, 1H), 7.69-8.0 (m, 4H), 4.49-4.58 (m, 2H), 3.99 (s, 1H), 2.32-2.33 (m, 1H), 2.03-2.05 (m, 1H), 1.45-1.70 (m, 9H), 1.15-1.31 (m, 1H). |
| (Example 34) | |
| 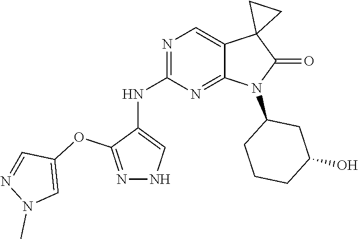 [SMILES: Cn1cc(Oc2n[nH]cc2Nc2ncc3c(n2)N([C@@H]2CCC[C@@H](O)C2)C(=O)C32CC2)cn1] | LC-MS m/z [M + H]⁺ = 437.25, ¹H NMR (400 MHz, DMSO- d₆) δ = 11.98 (s, 1H), 8.44 (s, 1H), 7.80 (s, 1H), 7.760-7.764 (m, 1H), 7.34 (s, 1H), 4.55-4.70 (m, 1H), 4.51-4.52 (d, J = 2.4 Hz, 1H), 4.029 (br-s, 1H), 3.75 (s, 3H), 2.41-2.50 (m, 1H), 2.15-2.25 (m, 1H), 1.45-1.75 (m, 9H), 1.28-1.40 (m, 1H). |
| (Example 35) | |
| 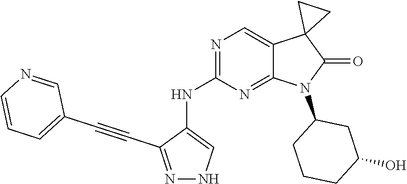 [SMILES: O=C1N([C@@H]2CCC[C@@H](O)C2)c2nc(Nc3c[nH]nc3C#Cc3cccnc3)ncc2C12CC2] | LC-MS m/z [M + H]⁺ = 441.90, ¹H NMR (400 MHz, DMSO- d₆) δ = 13.08-13.25 (br-s, 1H, rotamers), 9.00-9.17 (br-s, 1H, rotamers), 8.74 (br-s, 1H), 8.56-8.57 (m, 1H), 8.08 (br-s, 1H), 7.95-7.98 (m, 1H), 7.90 (s, 1H), 7.44-7.47 (m, 1H), 4.66-4.72 (m, 1H), 4.562-4.568 (d, J = 2.4 Hz, 1H), 4.05-4.15 (m, 1H), 2.49-2.501 (m, 1H), 2.21-2.25 (m, 1H), 1.23-1.78 (m, 10H). |
| (Example 36) | |
| 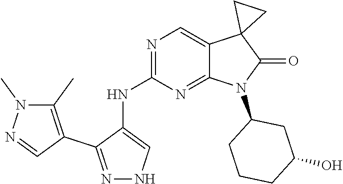 [SMILES: Cc1c(-c2n[nH]cc2Nc2ncc3c(n2)N([C@@H]2CCC[C@@H](O)C2)C(=O)C32CC2)cnn1C] | LC-MS m/z [M + H]⁺ = 435.50, ¹H NMR (400 MHz, DMSO- d₆) δ = 12.57 (br-s, 1H), 8.25 (br-s, 1H), 7.75 (br-s, 1H), 7.46 (s, 1H), 4.50-4.58 (m, 2H), 3.98-4.05 (m, 1H), 3.72 (s, 3H), 2.39 (s, 3H), 2.02-2.11 (m, 2H), 1.40-1.80 (m, 9H), 1.20-1.35 (m, 1H). |
| (Example 37) | |
| 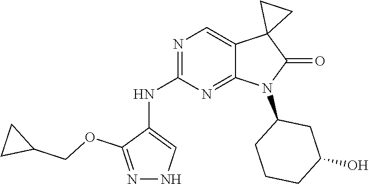 [SMILES: O=C1N([C@@H]2CCC[C@@H](O)C2)c2nc(Nc3c[nH]nc3OCC3CC3)ncc2C12CC2] | LC-MS m/z [M + H]⁺ = 440.26, ¹H NMR (400 MHz, DMSO-d₆) δ = 11.61 (s, 1H), 8.10 (s, 1H), 7.79 (s, 1H), 7.64 (s, 1H), 4.62-4.70 (m, 1H), 4.52-4.53 (d, J = 2.4 Hz, 1H), 4.058 (br-s, 1H), 3.91-3.93 (m, 2H), 2.41-2.50 (m, 1H), 2.15-2.25 (m, 1H), 1.25-1.75 (m, 10H), 1.28-1.40 (m, 1H), 1.15-1.25 (m, 2H), 0.49-0.53 (m, 2H), 0.25-0.30 (m, 2H). |
| (Example 38) | |
| 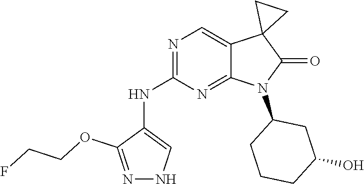 [SMILES: O=C1N([C@@H]2CCC[C@@H](O)C2)c2nc(Nc3c[nH]nc3OCCF)ncc2C12CC2] | LC-MS m/z [M + H]⁺ = 440.26, ¹H NMR (400 MHz, DMSO- d₆) δ = 11.72 (s, 1H), 8.17 (s, 1H), 7.67 (s, 1H), 4.63-4.77 (m, 3H), 4.52-4.526 (d, J = 2.4 Hz, 1H), 4.29-4.38 (m, 2H), 4.06 (br-s, 1H), 2.46-2.49 (m, 1H), 2.20-2.32 (m, 1H), 1.70-1.80 (m, 1H), 1.51-1.65 (m, 6H), 1.41-1.45 (m, 2H), 1.25-1.38 (m, 1H) |
| (Example 39) | |
| 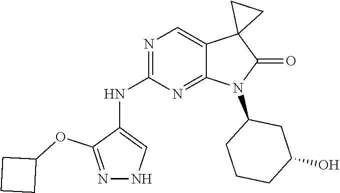 [SMILES: O=C1N([C@@H]2CCC[C@@H](O)C2)c2nc(Nc3c[nH]nc3OC3CCC3)ncc2C12CC2] | LC-MS m/z: [M + H]⁺ = 411.31 ¹H NMR (400 MHz, DMSO- d₆) δ = 11.613 (s, 1H),8.104 (br s, 1H) 7.792 (s, 1H), 7.760- 7.637 (s, 1H), 4.833-4.815 (m, 1H), 4.657-4.626 (t, J = 12.4 Hz 1H), 4.532-4.526 (d 1H), 4.1 (br s 1H), 2.497-2.328 (m 1H) 2.324-2.319 (m 1H), 2.312-2.305 (m 1H), 2.074-2.013 (m 2H), 1.757-1.462 (m 11H), 1.494-1.462 (m 1H) |
| (Example 40) | |
| 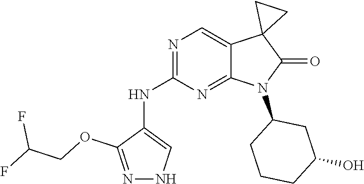 [SMILES: O=C1N([C@@H]2CCC[C@@H](O)C2)c2nc(Nc3c[nH]nc3OCC(F)F)ncc2C12CC2] | LC-MS m/z: [M + H]⁺ = 421.29 ¹H NMR (400 MHz, DMSO- d₆) δ = 11.853(s, 1H), 8.276 (br s 1H) 7.793 (s 1H), 7.696 (s, 1H), 6.491-6.482 (t J = 4.8 Hz 1H), 4.624-4.595 (t, J = 11.6 Hz 1H), 4.528 (s 1H), 4.410-4.432 (m 2H), 4.063 (s 1H), 2.497 (s 1H), 2.217-2.190 (m 1H), 1.753-1.507 (m 9H), 1.482-1.324 (m1H) |
| (Example 41) | |
| 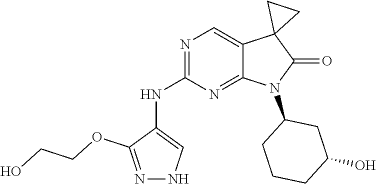 [SMILES: O=C1N([C@@H]2CCC[C@@H](O)C2)c2nc(Nc3c[nH]nc3OCCO)ncc2C12CC2] | LC-MS m/z: [M + H]⁺ = 401.21 ¹H NMR (400 MHz, DMSO-d₆) δ = 11.622 (s, 1H), 8.264 (br s 1H) 7.824 (s 1H), 7.727 (s, 1H), 4.878-4.864 (t J = 5.6 Hz 1H), 4.688- 4.658 (t, J = 12 Hz 1H), 4.554-4.547 (d J = 2.8 Hz 1H), 4.126-4.083 (m 3H), 3.686- 3.647 (m 2H), 2.467 (m 1H), 2.328-2.1810 (m 1H), 1.803- 1.737 (m 1H), 1.704-1.486 (m 6H), 1.470-1.462 (m 2H), 1.453-1443 (m 1H) |
| (Example 42) | |
| 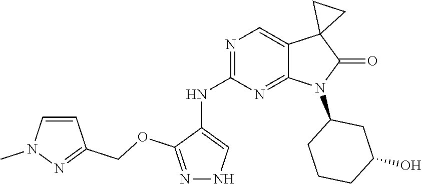 [SMILES: Cn1ccc(COc2n[nH]cc2Nc2ncc3c(n2)N([C@@H]2CCC[C@@H](O)C2)C(=O)C32CC2)n1] | LC-MS m/z: [M + H]⁺ = 451.41 ¹H NMR (400 MHz, DMSO- d₆) δ = 11.7 (s, 1H), 8.152 (s, 1H) 7.782 (s, 1H), 7.676-7.673 (d, J = 1.2 Hz 1H) 7.621-7.615 (d, J = 2.4Hz 1H) 6.524 (s, 1H), 6.286-6.273 (t J = 3.2 Hz 1H), 5.062 (s 2H), 4.620 (t, 1H), 4.531-4.524 (d J = 2.8 1H), 4.072 (s 1H), 3.808 (s 3H), 2.497-2.492 (m 1H), 2.333-2.323 (m1H), 1.753-1.429 (m 12H) |
| (Example 43) | |
| 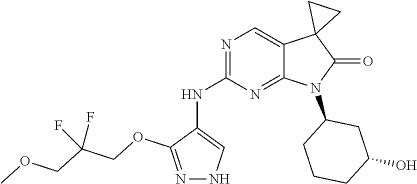 [SMILES: COCC(F)(F)COc1n[nH]cc1Nc1ncc2c(n1)N([C@@H]1CCC[C@@H](O)C1)C(=O)C21CC1] | LC-MS m/z: [M + H]⁺ = 464.24 ¹H NMR (400 MHz, DMSO- d₆) δ = 11.843 (s,1H), 8.431 (s, 1H) 7.816 (s, 1H), 7.741 (s, 1H), 4.682-4.651 (t, J = 12.4 1H), 4.545 (br s 1H), 4.431- 4.399 (t J = 13.6 Hz 2H), 3.240 (s 3H), 2.492 (m 1H), 2.337-2.319 (m1H), 1.761-1.460 (m 9H) |
| (Example 44) | |
| 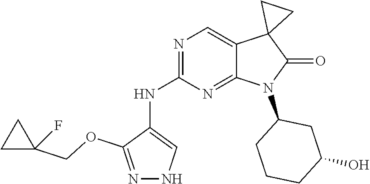 [SMILES: O=C1N([C@@H]2CCC[C@@H](O)C2)c2nc(Nc3c[nH]nc3OCC3(F)CC3)ncc2C12CC2] | LC-MS m/z: [M + H]⁺ = 429.24 ¹H NMR (400 MHz, DMSO- d₆) δ = 11.719 (s,1H), 8.155 (br s, 1H), 7.796 (s, 1H), 7.667 (s, 1H), 4.658-4.627 (t, J = 12.4 1H),4.529-4.522 (d J = 28 Hz 1H), 4.421-4.364 (d J = 22.8 Hz 2H), 4.064 (br s 1H), 2.455 (m 1H), 2.323-2.231 (m 1H) 1.780-1.464 (m 9H), 1.356- 1.336 (m 1H), 1.091-1.075 (m 2H), 0.8-0.6 (m 2H) |
| (Example 45) | |
| 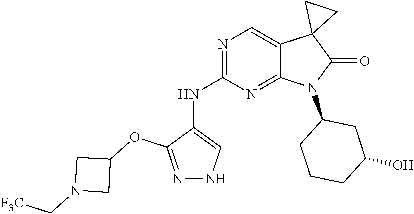 [SMILES: O=C1N([C@@H]2CCC[C@@H](O)C2)c2nc(Nc3c[nH]nc3OC3CN(CC(F)(F)F)C3)ncc2C12CC2] | LC-MS m/z: [M + H]⁺ = 494.24 ¹H NMR (400 MHz, DMSO- d₆) δ = 11.717 (s, 1H), 8.231 (s, 1H), 7.798 (s, 1H), 7.672 (s, 1H), 4.954-4.897 (m 1H), 4.657-4.533 (m 2H), 4.064 (s 1H), 3.787-3.766, (t J = 6.8 Hz 2H), 3.318-3.240 (m 4H), 2.492 (m 1H), 2.332-2.319 (m 1H) 1.758-1.506 (m 9H), 1.492-1.481 (m 1H), |
| (Example 46) | |
| 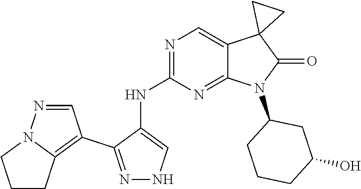 [SMILES: O=C1N([C@@H]2CCC[C@@H](O)C2)c2nc(Nc3c[nH]nc3-c3cnn4c3CCC4)ncc2C12CC2] | LC-MS m/z: [M + H]⁺ = 447.52 ¹H NMR (400 MHz, DMSO- d₆) δ = 12.62 (s, 1H), 8.356 (s, 1H), 7.760 (s, 1H), 7.607 (s, 1H), 7.515 (s 1H) 4.570-4.491 (m 2H), 4.084-4.020 (m 3H), 2.951-2.905 (t 2H), 2.530-2.510 (m 2H), 2.501-2.497 (m 1H), 2.328-2.108 (m 1H), 1.908-1.420 (m 9H), 1.355-1.236 (m 1H) |
| (Example 47) | |
| 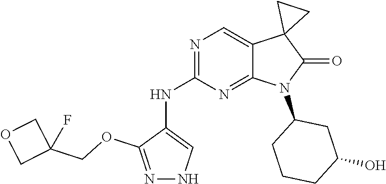 [SMILES: O=C1N([C@@H]2CCC[C@@H](O)C2)c2nc(Nc3c[nH]nc3OCC3(F)COC3)ncc2C12CC2] | LC-MS m/z: [M + H]⁺ = 445.42 ¹H NMR (400 MHz, DMSO-d₆) δ = 11.796 (s,1H), 8.303 (s, 1H), 7.805 (s, 1H), 7.713 (s, 1H), 4.722-4.487 (m 7H), 4.072 (br s 1H), 2.493-2.451 (m 1H) 2.251-2.161 (m 1H), 1.790-1.337 (m 12 H) |
| (Example 48) | |
| 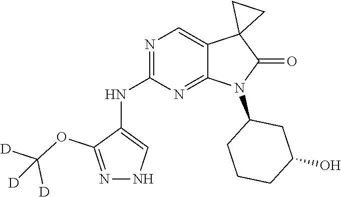 [SMILES: [2H]C([2H])([2H])Oc1n[nH]cc1Nc1ncc2c(n1)N([C@@H]1CCC[C@@H](O)C1)C(=O)C21CC1] | LC-MS m/z: [M + H]⁺ = 494.24 ¹H NMR (400 MHz, DMSO- d₆) δ = 11.632 (s, 1H), 8.185 (s, 1H), 7.782 (s, 1H), 7.645- 7.641 (d, J = 1.6 Hz 1H), 4.649-4.618 (t J = 12.4 1H), 4.526- 4.520 (d J = 2.4 Hz 1H), 4.064 (br s 1H), 2.505-2.457 (m 1H), 2.263-2.177 (m 1H), 1.786-1.459 (m 9H), 1.361-1.236 (m 1H) |
| (Example 49) | |
| 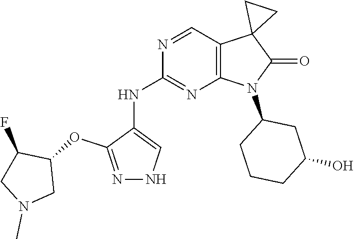 [SMILES: CN1C[C@@H](F)[C@H](Oc2n[nH]cc2Nc2ncc3c(n2)N([C@@H]2CCC[C@@H](O)C2)C(=O)C32CC2)C1] | LC-MS m/z (M + H)⁺: 458.24 ¹H NMR (400 MHz, DMSO-d₆) δ = 11.79 (s, 1H), 8.24 (s, 1H), 7.80 (s, 1H), 7.70 (s, 1H), 5.00-5.18 (m, 1H), 4.99-4.92 (m, 1H), 4.66-4.60 (m, 1H), 4.54 (brs, 1H), 4.06 (s, 1H), 3.18-3.16 (m, 1H), 2.82-2.65 (m, 2H), 2.49-2.42 (obs, 1H), 2.40-2.37 (m, 1H), 2.31-2.12 (m, 4H), 1.61-1.49 (m, 9H), 1.47-1.45 (m, 2H), |
| (Example 50) | |
| 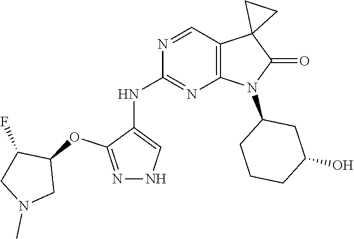 [SMILES: CN1C[C@H](Oc2n[nH]cc2Nc2ncc3c(n2)N([C@@H]2CCC[C@@H](O)C2)C(=O)C32CC2)[C@@H](F)C1] | LC-MS m/z (M + H)⁺: 458.24 ¹H NMR (400 MHz, DMSO-d₆) δ = 11.80 (s, 1H), 8.26 (s, 1H), 7.80 (s, 1H), 7.71 (s, 1H), 5.01-5.22 (m, 1H), 4.97-4.96 (m, 1H), 4.63-4.60 (m, 1H), 4.54-4.53 (brs, 1H), 4.06 (s, 1H), 3.31-3.16 (m, 1H), 2.86- 2.67 (m, 2H), 2.50-2.42 (obs, 1H), 2.40-2.37 (m, 1H), 2.41- 2.12 (m, 4H), 1.81-1.49 (m, 9H), 1.47-1.20 (m, 2H), |
| (Example 51) | |
| 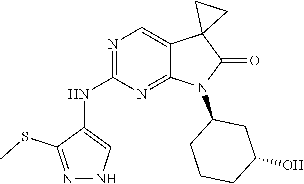 [SMILES: CSc1n[nH]cc1Nc1ncc2c(n1)N([C@@H]1CCC[C@@H](O)C1)C(=O)C21CC1] | LC-MS m/z (M + H)⁺: 387.24 ¹H NMR (400 MHz, DMSO-d₆) δ = 12.94-12.75 (m, 1H), 8.35-8.19 (m, 1H), 7.85-7.72 (m, 2H), 4.68-4.65 (m, 1H), 4.54 (brs, 1H), 4.06 (s, 1H), 2.49-2.45 (obs, 1H), 2.34 (s, 3H), 2.25-2.19 (m, 1H), 1.80-1.45 (m, 9H). |
| (Example 52) | |
| 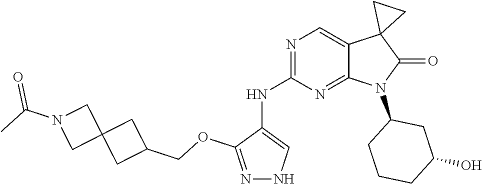 [SMILES: CC(=O)N1CC2(CC(COc3n[nH]cc3Nc3ncc4c(n3)N([C@@H]3CCC[C@@H](O)C3)C(=O)C43CC3)C2)C1] | LCMS: 508.37 [M + H]⁺ (90% purity). ¹H NMR (400 MHz, DMSO-d₆) δ = 11.64 (brs, 1H), 8.11 (s, 1H), 7.79 (s, 1H), 7.70-7.63 (m, 1H), 4.53 (brs, 1H), 4.07-3.94 (m, 5H), 3.77- 3.68 (m, 2H), 2.56-2.42 (obs, 2H), 2.25-2.20 (m, 3H), 2.01- 1.97 (m, 2H), 1.69-1.32 (m, 13H). |
| (Example 53) | |
| 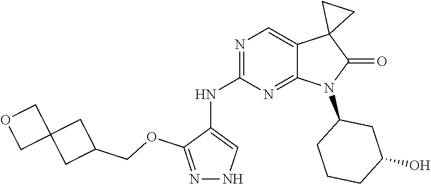 [SMILES: O=C1N([C@@H]2CCC[C@@H](O)C2)c2nc(Nc3c[nH]nc3OCC3CC4(COC4)C3)ncc2C12CC2] | LCMS: 467.38 [M + H]⁺ (97%) ¹H NMR (400 MHz, DMSO-d₆) δ = 11.63 (brs, 1H), 8.08 (s, 1H), 7.79 (s, 1H), 7.62 (s, 1H), 4.65-4.53 (m, 1H), 4.51 (brs, 1H), 4.42 (s, 2H), 4.05-3.98 (m, 3H), 2.44-2.42 (obs, 2H), 2.29-2.19 (m, 3H), 2.00-1.95 (m, 2H), 1.75-1.43 (m, 9H), 1.33-1.30 (m, 2H). |
| (Example 54) | |
| 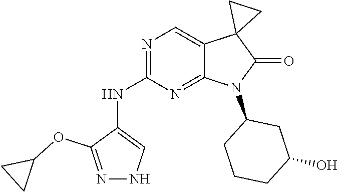 [SMILES: O=C1N([C@@H]2CCC[C@@H](O)C2)c2nc(Nc3c[nH]nc3OC3CC3)ncc2C12CC2] | LC-MS: 397.33 [M + H]⁺ (99%) ¹H NMR (400 MHz, DMSO- d₆) δ = 11.71 (brs, 1H), 8.14 (brs, 1H), 7.78 (s, 1H), 7.67 (s, 1H), 4.65-4.54 (m, 1H), 4.53 (brs, 1H), 4.06-4.01 (m, 2H), 2.49-2.46 (obs, 1H), 2.35-2.21 (m, 1H), 1.85-1.42 (m, 9H), 1.40-1.31 (m, 1H), 0.68-0.58 (m, 4H). |
| (Example 55) | |
| 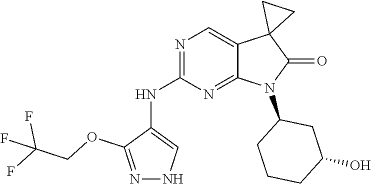 [SMILES: O=C1N([C@@H]2CCC[C@@H](O)C2)c2nc(Nc3c[nH]nc3OCC(F)(F)F)ncc2C12CC2] | LC-MS: 439.32 [M + H]⁺ (99% Purity). ¹H NMR (400 MHz, DMSO-d₆) δ = 11.94 (brs, 1H), 8.32 (s, 1H), 7.79 (s, 1H), 7.70 (s, 1H), 4.79-4.72 (m, 2H), 4.65-4.52 (m, 2H), 4.06 (brs, 1H), 2.46-2.42 (obs, 1H), 2.24-2.15 (m, 1H), 1.78-1.46 (m, 9H), 1.34-1.31 (m, 1H). |
| (Example 56) | |
| 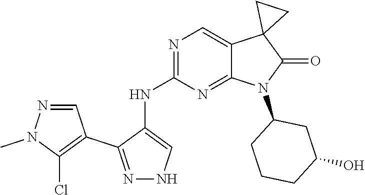 [SMILES: Cn1ncc(-c2n[nH]cc2Nc2ncc3c(n2)N([C@@H]2CCC[C@@H](O)C2)C(=O)C32CC2)c1Cl] | LC-MS: 455.24 [M + H]⁺ (98%) ¹H NMR (400 MHz, DMSO- d₆) δ = 12.75 (brs, 1H), 8.37 (s, 1H), 7.85-7.64 (m, 3H), 4.65- 4.50 (m, 2H), 4.03 (s, 1H), 3.80 (s, 3H), 4.456-4.451 (obs, 1H), 2.11-2.10 (m, 1H), 1.72-1.45 (m, 9H), 1.31-1.23 (m, 1H). |
| (Example 57) | |
| 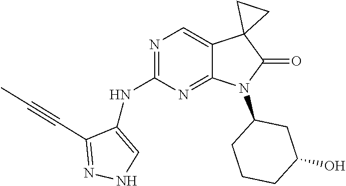 [SMILES: CC#Cc1n[nH]cc1Nc1ncc2c(n1)N([C@@H]1CCC[C@@H](O)C1)C(=O)C21CC1] | LC-MS m/z [M + H]+ = 379.25, ¹H NMR (400 MHz, DMSO- d₆) δ = 12.77 (br-s, 1H), 8.20-8.65 (br-s, 1H), 7.75-8.00 (m, 2H), 4.62-4.72 (m, 1H), 4.56 (d, J = 1.4 Hz, 1H), 4.09 (s, 1H), 2.45-2.52 (m, 1H), 2.18-2.30 (m, 1H), 2.07 (s, 3H), 1.45-1.82 (m, 9H), 1.32-1.44 (m, 1H). |
| (Example 58) | |
| 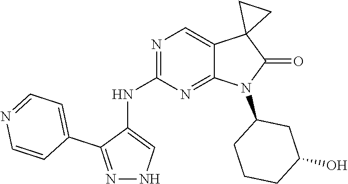 [SMILES: O=C1N([C@@H]2CCC[C@@H](O)C2)c2nc(Nc3c[nH]nc3-c3ccncc3)ncc2C12CC2] | LC-MS m/z [M + H]+ = 418.28, ¹H NMR (400 MHz, DMSO-d₆) δ = 13.07-13.42 (m, 1H), 8.45-8.85 (m, 3H), 7.58-7.90 (m, 4H), 4.43-4.62 (m, 2H), 3.95 (s, 1H), 1.88-2.32 (m, 2H), 1.5-1.22 (m, 4H), 1.12-1.49 (m, 6H). |
| (Example 59) | |
| 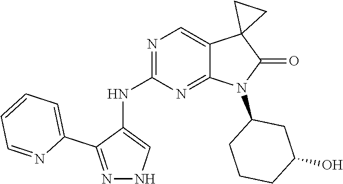 [SMILES: O=C1N([C@@H]2CCC[C@@H](O)C2)c2nc(Nc3c[nH]nc3-c3ccccn3)ncc2C12CC2] | LC-MS m/z [M + H]⁺ = 418.22, ¹H NMR (400 MHz, DMSO- d₆) δ = 13.06 (br-s, 1H), 10.37 (br-s, 1H), 8.67 (d, J = 4.8 Hz, 1H), 8.32 (s, 1H), 8.04 (d, J = 7.6 Hz, 1H), 7.96 (s, 1H), 7.88-7.95 (m, 1H), 7.32-7.37 (m, 1H) 4.70-4.82 (m, 1H), 4.65 (d, J = 2.4 Hz, 1H), 4.16 (s, 1H), 2.50-2.60 (m, 1H), 2.25- 2.40 (m, 1H), 1.58-1.92 (m, 7H), 1.40-1.60 (m, 3H). |
| (Example 60) | |
| 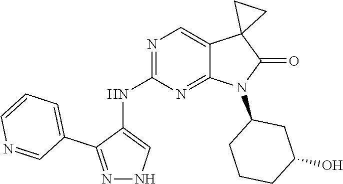 [SMILES: O=C1N([C@@H]2CCC[C@@H](O)C2)c2nc(Nc3c[nH]nc3-c3cccnc3)ncc2C12CC2] | LC-MS m/z [M + H]+ = 418.24, ¹H NMR (400 MHz, DMSO- d₆) δ = 12.87 (br-s, 1H), 8.92 (d, J = 1.2 Hz, 1H), 8.65 (s, 1H), 8.45 (dd, J = 1.6, 4.8 Hz, 1H), 8.05-8.10 (m, 1H), 7.77 (s, 1H), 7.40 (dd, J = 4.8, 7.6 Hz, 1H), 4.40-4.62 (m, 2H), 3.96 (br-s, 1H), 2.15-2.35 (m ,1H), 1.88-2.10 (m, 1H), 1.38- 1.75 (m, 9H), 1.10-1.30 (m, 1H). |
| (Example 61) | |
| 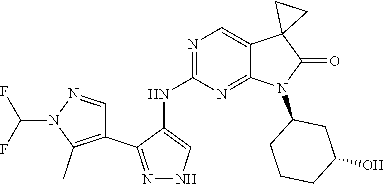 [SMILES: Cc1c(-c2n[nH]cc2Nc2ncc3c(n2)N([C@@H]2CCC[C@@H](O)C2)C(=O)C32CC2)cnn1C(F)F] | LC-MS m/z [M + H]+ = 471.30, 1H NMR (400 MHz, DMSO- d₆) δ = 12.80 (s, 1H), 8.50 (s, 1H), 7.64-7.98 (m, 4H), 4.48- 4.65 (m, 2H), 3.98-4.10 (m, 1H), 2.57 (s, 2H), 2.30-2.40 (m, 1H), 1.98-2.20 (m, 1H), 1.40-1.78 (m, 9H), 1.18-1.38 (m, 1H). |
| (Example 62) | |
| 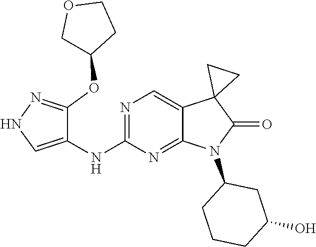 [SMILES: O=C1N([C@@H]2CCC[C@@H](O)C2)c2nc(Nc3c[nH]nc3O[C@@H]3CCOC3)ncc2C12CC2] | LC-MS m/z [M + H]⁺ = 427.31, ¹H NMR (400 MHz, DMSO-d₆) δ = 11.69 (s, 1H), 8.16 (s, 1H), 7.80 (s, 1H), 7.68 (d, J = 1.6 Hz, 1H), 5.09-5.15 (m, 1H), 4.63 (t, J = 11.6 Hz, 1H), 4.53 (d, J = 2.4 Hz, 1H), 4.06 (s, 1H), 3.77-3.85 (m, 3H), 3.68- 3.74 (m, 1H), 2.42-2.52 (m, 1H), 2.01-2.28 (m, 3H), 1.40- 1.82 (m, 9H), 1.20-1.40 (m, 1H). |
| (Example 63) | |
| 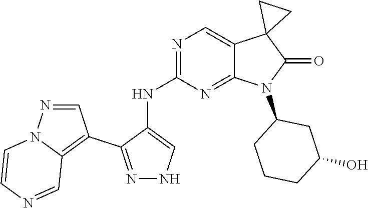 [SMILES: O=C1N([C@@H]2CCC[C@@H](O)C2)c2nc(Nc3c[nH]nc3-c3cnn4ccncc34)ncc2C12CC2] | LC-MS m/z [M + H]⁺ = 458.31, ¹H NMR (400 MHz, DMSO- d₆) δ = 12.92-13.11 (m, 1H), 9.27-9.56 (m, 1H), 8.70-8.82 (m, 2H), 8.19-8.36 (m, 1H), 7.63-7.96 (m, 3H), 4.38-4.62 (m, 2H), 3.70-4.02 (m, 1H), 1.78-2.30 (m, 2H), 1.04-1.72 (m, 10H). |
| (Example 64) | |
| 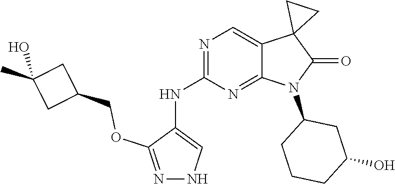 [SMILES: C[C@]1(O)C[C@H](COc2n[nH]cc2Nc2ncc3c(n2)N([C@@H]2CCC[C@@H](O)C2)C(=O)C32CC2)C1] | LC-MS m/z [M + H]+ = 455.33, ¹H NMR (400 MHz, DMSO-d₆) δ = 11.61 (s, 1H), 8.10 (s, 1H), 7.80 (s, 1H), 7.66 (d, J = 1.2 Hz, 1H), 4.85 (s, 1H), 4.63 (t, J = 12.4 Hz, 1H), 4.54 (d, J = 2.8 Hz, 1H), 4.05 (d, J = 6.4 Hz, 1H), 2.42-2.52 (m, 1H), 2.12-2.38 (m, 2H), 1.95-2.05 (m, 2H), 1.42-1.82 (m, 11H), 1.28-1.40 (m, 1H), 1.21 (s, 3H). |
| (Example 65) | |
| 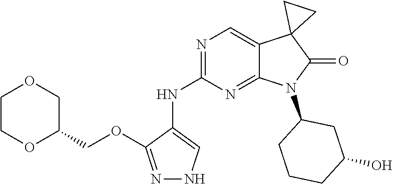 [SMILES: O=C1N([C@@H]2CCC[C@@H](O)C2)c2nc(Nc3c[nH]nc3OC[C@H]3COCCO3)ncc2C12CC2] | LC-MS m/z [M + H]⁺ = 457.31, ¹H NMR (400 MHz, DMSO-d₆) δ = 11.69 (s, 1H), 8.24 (s, 1H), 7.80 (s, 1H), 7.69 (d, J = 1.6Hz, 1H), 4.58-4.70 (m, 1H), 4.53 (d, J = 2.8 Hz, 1H), 4.01-4.12 (m, 3H), 3.72-3.90 (m, 3H), 3.54-3.68 (m, 2H), 3.42-3.52 (m, 1H), 3.30-3.40 (m, 1H), 2.42-2.52 (m, 1H), 2.15-2.30 (m, 1H), 1.42-1.82 (m, 9H), 1.28-1.41 (m, 1H). |
| (Example 66) | |
| 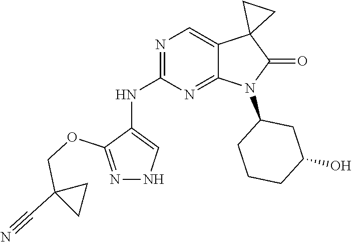 [SMILES: N#CC1(COc2n[nH]cc2Nc2ncc3c(n2)N([C@@H]2CCC[C@@H](O)C2)C(=O)C32CC2)CC1] | LC-MS m/z [M + H]+ = 436.35, ¹H NMR (400 MHz, DMSO- d₆) δ = 11.77 (s, 1H), 8.22 (s, 1H), 7.80 (s, 1H), 7.68 (s, 1H), 4.63 (t, J = 11.6 Hz, 1H), 4.53 (d, J = 2.4 Hz, 1H), 4.13 (s, 2H), 4.07 (s, 1H), 2.45-2.52 (m, 1H), 2.15-2.28 (m, 1H), 1.40-1.80 (m, 9H), 1.28-1.40 (m, 3H), 1.14-1.27 (m, 2H). |
| (Example 67) | |
| 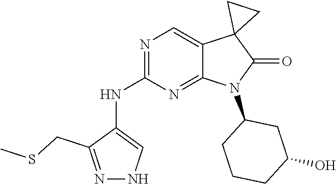 [SMILES: CSCc1n[nH]cc1Nc1ncc2c(n1)N([C@@H]1CCC[C@@H](O)C1)C(=O)C21CC1] | LC-MS m/z [M + H]+ = 401.30, ¹H NMR (400 MHz, DMSO- d₆) δ = 12.40 (br-s, 1H), 8.69 (s, 1H), 7.80-8.04 (m, 2H), 4.69 (t, J = 12.4 Hz, 1H), 4.57 (s, 1H), 4.10 (s, 1H), 3.81 (s, 2H), 2.45-2.52 (m, 1H), 2.16-2.30 (m, 1H), 1.97 (s, 3H), 1.32-1.85 (m, 10H). |
| (Example 68) | |
| 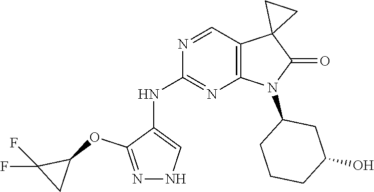 [SMILES: O=C1N([C@@H]2CCC[C@@H](O)C2)c2nc(Nc3c[nH]nc3O[C@H]3CC3(F)F)ncc2C12CC2] | LC-MS m/z [M + H]⁺ = 433.24, ¹H NMR (400 MHz, DMSO-d₆) δ = 11.91 (br-s, 1H), 8.29 (s, 1H), 7.79 (s, 1H), 7.71 (s, 1H), 4.42-4.72 (m, 3H), 4.07 (s, 1H), 2.40-2.52 (m, 1H), 2.12-2.30 (m, 1H), 1.90-2.00 (m, 1H), 1.28-1.80 (m, 11H). |
| (Example 69) | |
| 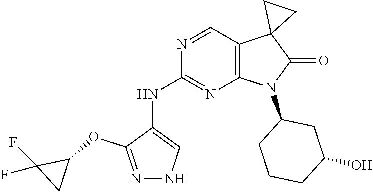 [SMILES: O=C1N([C@@H]2CCC[C@@H](O)C2)c2nc(Nc3c[nH]nc3O[C@@H]3CC3(F)F)ncc2C12CC2] | LC-MS m/z [M + H]⁺ = 433.37, ¹H NMR (400 MHz, DMSO-d₆) δ = 11.90 (br-s, 1H), 8.29 (s, 1H), 7.79 (s, 1H), 7.71 (s, 1H), 4.42-4.70 (m, 3H), 4.07 (s, 1H), 2.40-2.52 (m, 1H), 2.15-2.29 (m, 1H), 1.90-2.01 (m, 1H), 1.28-1.82 (m, 11H). |
| (Example 70) | |
| 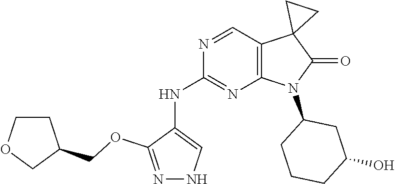 [SMILES: O=C1N([C@@H]2CCC[C@@H](O)C2)c2nc(Nc3c[nH]nc3OC[C@@H]3CCOC3)ncc2C12CC2] | LC-MS m/z [M + H]⁺ = 441.26, ¹H NMR (400 MHz, DMSO- d₆) δ = 11.66 (s, 1H), 8.19 (s, 1H), 7.79 (s, 1H), 7.67 (d, J = 1.2 Hz, 1H), 4.58-4.70 (m, 1H), 4.52-4.56 (m, 1H), 4.03-4.10 (m, 2H), 3.97-4.02 (m, 1H), 3.68-3.75 (m, 2H), 3.59-3.66 (m, 1H), 3.50-3.56 (m, 1H), 2.58-2.68 (m, 1H), 2.42-2.50 (m, 1H), 2.15-2.26 (m, 1H), 1.90-2.01 (m, 1H), 1.40-1.80 (m, 10H), 1.28-1.39 (m, 1H). |
| (Example 71) | |
| 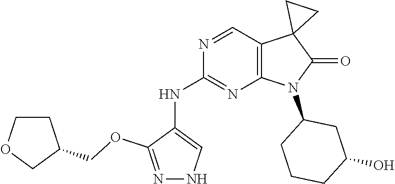 [SMILES: O=C1N([C@@H]2CCC[C@@H](O)C2)c2nc(Nc3c[nH]nc3OC[C@H]3CCOC3)ncc2C12CC2] | LC-MS m/z [M + H]⁺ = 441.26, ¹H NMR (400 MHz, DMSO-d₆) δ = 11.66 (s, 1H), 8.19 (s, 1H), 7.79 (s, 1H), 7.67 (s, 1H), 4.58-4.70 (m, 1H), 4.53 (d, J = 2.4 Hz, 1H), 4.03-4.10 (m, 2H), 3.96-4.02 (m, 1H), 3.69- 3.75 (m, 2H), 3.59-3.66 (m, 1H), 3.50-3.55 (m, 1H), 2.59- 2.69 (m, 1H), 2.42-2.50 (m, 1H), 2.15-2.28 (m, 1H), 1.90- 2.01 (m, 1H), 1.41-1.80 (m, 10H), 1.28-1.40 (m, 1H). |
| (Example 72) | |
| 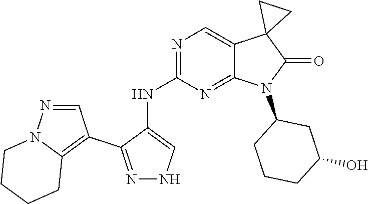 [SMILES: O=C1N([C@@H]2CCC[C@@H](O)C2)c2nc(Nc3c[nH]nc3-c3cnn4c3CCCC4)ncc2C12CC2] | LC-MS m/z [M + H]⁺ = 461.36, ¹H NMR (400 MHz, DMSO-d₆) δ = 8.31 (s, 1H), 7.76 (s, 1H), 7.67 (s, 1H), 7.52 (s, 1H), 4.40-4.70 (m, 2H), 3.98-4.10 (m, 3H), 2.82-2.90 (m, 2H), 2.30-2.45 (m, 1H), 2.02-2.30 (m, 1H), 1.90-1.99 (m, 2H), 1.40-1.80 (m, 11H), 1.18-1.32 (m, 1H). |
| (Example 73) | |
| 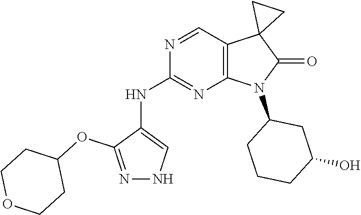 [SMILES: O=C1N([C@@H]2CCC[C@@H](O)C2)c2nc(Nc3c[nH]nc3OC3CCOCC3)ncc2C12CC2] | LC-MS m/z [M + H]⁺ = 441.31, ¹H NMR (400 MHz, DMSO-d₆) δ = 11.67 (s, 1H), 8.15 (s, 1H), 7.80 (s, 1H), 7.69 (s, 1H), 4.58-4.72 (m, 2H), 4.54 (s, 1H), 4.07 (s, 1H), 3.82-3.90 (m, 2H), 3.36-3.48 (m, 2H), 2.45-2.52 (m, 1H), 2.15-2.28 (m, 1H), 1.92-2.02 (m, 2H), 1.40-1.82 (m, 11H), 1.28-1.41 (m, 1H). |
| (Example 74) | |
| 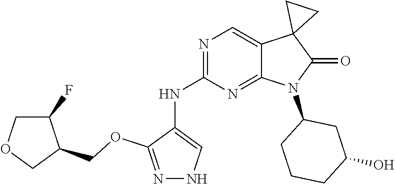 [SMILES: O=C1N([C@@H]2CCC[C@@H](O)C2)c2nc(Nc3c[nH]nc3OC[C@H]3COC[C@H]3F)ncc2C12CC2] | LC-MS m/z [M + H]⁺ = 459.30, ¹H NMR (400 MHz, DMSO-d₆) δ = 11.69 (s, 1H), 8.25 (s, 1H), 7.81 (s, 1H), 7.70 (s, 1H), 5.38-5.62 (m, 1H), 4.59-4.70 (m, 1H), 4.54 (s, 1H), 4.30-4.38 (m, 1H), 4.12-4.20 (m, 1H), 4.18 (s, 1H), 3.80-3.99 (m, 3H), 3.52-3.60 (m, 1H), 2.70-2.90 (m, 1H), 2.42-2.50 (m, 1H), 2.15-2.29 (m, 1H), 1.40-1.80 (m, 9H), 1.28-1.40 (m, 1H). |
| (Example 75) | |
| 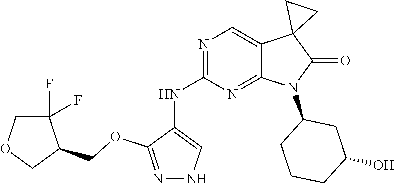 [SMILES: O=C1N([C@@H]2CCC[C@@H](O)C2)c2nc(Nc3c[nH]nc3OC[C@H]3COCC3(F)F)ncc2C12CC2] | LC-MS m/z [M + H]⁺ = 477.35, ¹H NMR (400 MHz, DMSO-d₆) δ = 11.75 (s, 1H), 8.23 (s, 1H), 7.80 (s, 1H), 7.70 (d, J = 1.2 Hz, 1H), 4.59-4.70 (m, 1H), 4.54 (d, J = 2.78 Hz, 1H), 4.30-4.38 (m, 1H), 4.12-4.20 (m, 2H), 4.07 (s, 1H), 3.92-4.02 (m, 1H), 3.80-3.91 (m, 2H), 2.96-3.10 (m, 1H), 2.42-2.50 (m, 1H), 2.15-2.28 (m, 1H), 1.28-1.82 (m, 10H). |
| (Example 766) | |
| 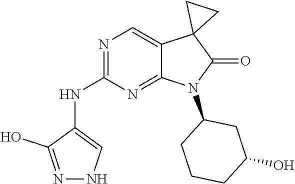 [SMILES: O=C1N([C@@H]2CCC[C@@H](O)C2)c2nc(Nc3c[nH]nc3O)ncc2C12CC2] | LC-MS m/z [M + H]+ = 357.48, ¹H NMR (400 MHz, DMSO- d6) δ = 11.5 (br-s, 1H), 9.32 (br-s, 1H), 7.80 (br-s, 1H), 7.51 (br-s, 1H), 4.57-4.70 (m, 1H), 4.09 (s, 1H), 2.45-2.52 (m, 1H), 2.18-2.30 (m, 1H), 1.64-1.76 (m, 6H), 1.51-1.56 (m, 3H), 1.34-1.41 (m, 1H). |
| (Example 77) | |
| 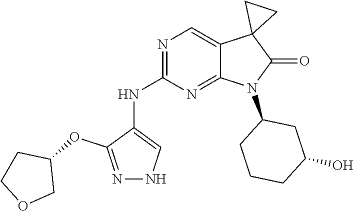 [SMILES: O=C1N([C@@H]2CCC[C@@H](O)C2)c2nc(Nc3c[nH]nc3O[C@H]3CCOC3)ncc2C12CC2] | LC-MS m/z [M + H]+ = 427.25, ¹H NMR (400 MHz, DMSO- d6) δ = 11.69 (br-s, 1H), 8.15 (br-s, 1H), 7.79 (br-s, 1H), 7.68 (d, J = 1.6 Hz, 1H), 5.12 (d, J = 5.6 Hz, 1H), 4.52-4.68 (m, 1H), 4.02-4.1 (br-s, 1H), 3.78-3.85 (m, 3H), 3.68-3.75 (m, 1H), 2.42-2.5 (m, 1H), 2.12-2.25 (m, 2H), 1.98-2.05 (m, 1H), 1.42-1.78 (m, 9H), 1.25-1.4(m, 1H). |
| (Example 78) | |
| 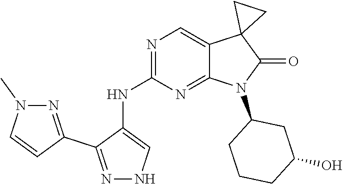 [SMILES: Cn1ccc(-c2n[nH]cc2Nc2ncc3c(n2)N([C@@H]2CCC[C@@H](O)C2)C(=O)C32CC2)n1] | LC-MS m/z [M + H]+ = 421.60, ¹H NMR (400 MHz, DMSO- d6) δ = 12.69-13.08 (d, J = 156 Hz, 1H), 8.85-9.24 (d, J = 156 Hz, 1H), 8.13-8.20 (d, J = 28 Hz, 1H), 7.91-7.94 (d, J = 12 Hz, 1H), 7.79-7.81 (d, J = 8 Hz, 1H), 6.52-6.63 (d, J = 44 Hz, 1H), 4.60-4.74 (m, 2H), 4.15 (br-s, 1H), 3.95 (s, 3H), 2.54-2.57 (m, 1H), 2.28-2.37 (m, 1H), 1.66-1.93 (m, 10H). |
| (Example 79) | |
| 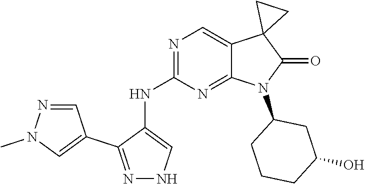 [SMILES: Cn1cc(-c2n[nH]cc2Nc2ncc3c(n2)N([C@@H]2CCC[C@@H](O)C2)C(=O)C32CC2)cn1] | LC-MS m/z [M + H]+ = 421.60, ¹H NMR (400 MHz, DMSO- d6) δ = 12.54-12.77 (d, J = 92 Hz, 1H), 8.39-8.43 (m, 1H), 7.48-7.87 (m ,4H), 4.49-4.58 (m, 2H), 4.00 (br-s, 1H), 3.83 (s, 3H), 2.32-2.40 (m, 1H), 2.07-2.08 (m, 1H), 1.26-1.70 (m, 9H), 0.90-0.94 (m, 1H). |
| (Example 80) | |
| 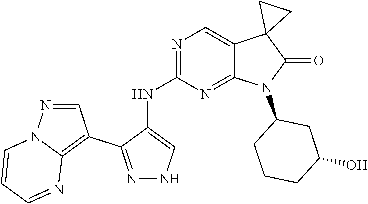 [SMILES: O=C1N([C@@H]2CCC[C@@H](O)C2)c2nc(Nc3c[nH]nc3-c3cnn4cccnc34)ncc2C12CC2] | LC-MS m/z [M + H]⁺ = 458.26 [M + H]⁺, ¹H NMR (400 MHz, DMSO-d₆) δ = 12.94-12.76 (m, 1H), 10.71-10.16 (m, 1H), 9.22 (d, 6.4 Hz, 1H), 8.59 (s, 2H), 8.27-8.13 (m, 1H), 7.91 (s, 1H), 7.15 (s, 1H), 4.72-4.62 (m, 2H), 4.16 (s, 1H), 2.50- 2.49 (m, 1H), 2.33-2.32 (m, 1H), 1.83-1.48 (m, 10H). |
| (Example 81) | |
| 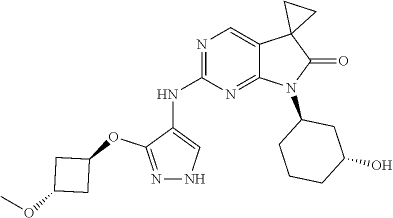 [SMILES: CO[C@H]1C[C@H](Oc2n[nH]cc2Nc2ncc3c(n2)N([C@@H]2CCC[C@@H](O)C2)C(=O)C32CC2)C1] | LC-MS m/z [M + H]⁺ = 441.32, ¹H NMR (400 MHz, DMSO-d₆) δ = 11.64 (brs, 1H), 8.15 (brs, 1H), 7.79 (s, 1H), 7.65 (s, 1H), 4.94 (q, 1H, 2.0 Hz), 4.65-4.52 (m, 2H), 4.06-3.99 (m, 2H), 3.13 (s, 3H), 2.49 (obs, 1H), 2.28 (t, 5.6 Hz, 4H), 2.19-.17 (m, 1H), 1.79-1.32 (m, 10H). |
| (Example 82) | |
| 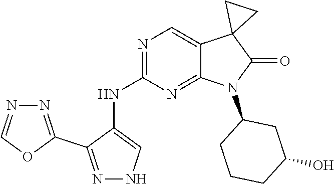 [SMILES: O=C1N([C@@H]2CCC[C@@H](O)C2)c2nc(Nc3c[nH]nc3-c3nnco3)ncc2C12CC2] | LC-MS m/z [M + H]⁺ = 409.32, ¹H NMR (400 MHz, DMSO- d₆) δ = 13.58 (s, 1H), 9.32 (s, 1H), 8.72 (s, 1H), 8.30 (s, 1H), 7.97 (s, 1H), 4.68-4.78 (m, 1H), 4.63 (s, 1H), 4.13 (s, 1H), 2.42-2.52 (m, 1H), 2.18-2.32 (m, 1H), 1.53-1.90 (m, 7H), 1.50-1.53 (m, 2H), 1.39-1.50 (m, 1H). |
| (Example 83) | |
| 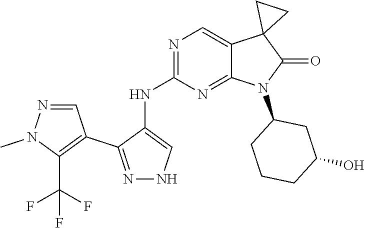 [SMILES: Cn1ncc(-c2n[nH]cc2Nc2ncc3c(n2)N([C@@H]2CCC[C@@H](O)C2)C(=O)C32CC2)c1C(F)(F)F] | LC-MS m/z [M + H]+ = 489.30, ¹H NMR (400 MHz, DMSO- d₆) δ = 12.81 (br-s, 1H), 8.39-8.45 (m, 1H), 7.65-7.92 (m, 3H), 4.58-4.65 (m, 1H), 4.52 (d, J = 2.8 Hz, 1H), 3.99-4.01 (m, 4H), 2.40-2.49 (m, 1H), 2.11-2.20 (m, 1H), 1.27-1.77 (m, 10H). |
| (Example 84) | |
| 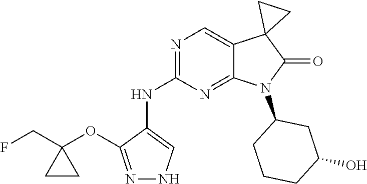 [SMILES: O=C1N([C@@H]2CCC[C@@H](O)C2)c2nc(Nc3c[nH]nc3OC3(CF)CC3)ncc2C12CC2] | LC-MS m/z [M + H]⁺ = 429.33 ¹H NMR (400 MHz, DMSO- d₆): δ = 11.82 (brs, 1H), 8.13 (s, 1H), 7.80 (s, 1H), 7.71 (s, 1H), 4.80-4.54 (m, 4H), 4.07 (s, 1H), 2.49 (bs, 1H), 2.25- 2.17 (m, 1H), 1.76-1.50 (m, 9H), 1.46-1.34 (m, 1H), 1.05- 1.00 (m, 2H), 0.88-0.85 (m, 2H). |
| (Example 85) | |

### CDKi Biochemical Assays

Biochemical assays measured the inhibitory effects of compounds in this disclosure on the enzymatic activity of CDK enzyme in complex with Cyclin protein partner by phosphorylation of Ser-780 (S780) on retinoblastoma protein peptide (RB1) in the presence of 1 mM adenosime-5-triphosphate (ATP) and varying concentration of test compound in 20 mM 2-[4-(2-hydroxyethyl)paperazin-1-yl] ethanesulfonic acid (HEPES), pH 7.5, 10 mM MgCl₂, 1 mM dithiothreitol (DTT), 0.01% bovine serum albumin (BSA), 0.005% Tween 20. Total Reaction volume of 10 μL proceeded for 60 minutes at room temperature (25° C.) and were quenched with 5 μL of 200 mM 2′,2″,2′″-(Ethane-1,2-diyldinitrilo)tetraacetic acid (EDTA) pH 8.0 before addition of 5 μL detection solution containing 100 nM fluorophore conjugate streptavidin allophycocyanin (SA-APC), 2 nM Europium labelled Anti-p-RB(S780)-K (Perkin Elmer, 64CUSKAY), 50 mM HEPES, pH 7.5, 400 mM potassium fluoride (KF), 0.1% BSA, and 0.01% Tween-20. Phosphorylation of S780 on RB1 peptide (His-MBP-RB1[773-924]-SEQ ID NO. 9) was detected by TR-FRET after 3 hour incubation with detection solution. Percent phosphorylation activity was plotted against log concentration of compound to generate an apparent ICSO. The following CDK enzyme in complex with different cyclin proteins and protein peptide substrate were used in these assays:
CDK1/CyclinB1, Avi-tag, 10 pM used in the assay
CDK2/CyclinE1, Avi-tag, 100 pM used in the assay
CDK4/cyclinD1, Avi-tag, 20 pM used in the assay
CDK6/cyclinD3, Avi-tag, 10 pM used in the assay
His-MBP-RB1[773-924], Avi-tag, 200 nM used in the assay

The avi-tag is a C-terminal fusion on the CDK1/2/4/6 sequence in each of these two-protein complexes. The known avi-tag sequence, GLNDIFEAQKIEWHE (SEQ ID NO. 10), is a substrate for E. coli biotin ligase BirA, which covalently attaches a biotin molecule to the e-amino group of the lysine in that sequence. The avi-tag is attached with an 8 amino-acid linker sequence GGSGGGGS (SEQ ID NO. 11), resulting in the full C-terminal fusion

| (SEQ ID NO. 12) | |
|---|---|
| | GGSGGGGSGLNDIFEAQKIEWHE |

The sequences of the recombinant proteins used here are provided below.

Recombinant proteins were generated as N-terminal fusions of 9× His tag—E. coli Maltose Binding Protein (MBP), with or without a C-terminal Avi-tag.¹ CDK2 and His-MBP-RB1[773-924]-c-avi were expressed in E. coli while the remainder were expressed in Sf21 insect cells. Genes were synthesized commercially (GeneArt and Twist Bioscience) using codon frequencies appropriate to the respective organisms and proprietary codon optimization strategies, and inserted into pET 24 (E. coli) or pDEST8 (insect) vectors already containing the 9× His-MBP tag using standard methods of PCR and Gibson Assembly.² E. coli expression was performed by auto-induction overnight at 18 or 21 C. For insect cell expression, bacmids and viruses were created,³ then used to synchronously infect Sf21 insect cells with harvest at 40-48 hrs., per standard protocols.⁴

For CDK2/Cyclin E and CDK1/Cyclin B, the CDK and Cyclin proteins were expressed separately, lysed by sonication and clarified by centrifugation. The fusion proteins were each separately purified by immobilized metal ion affinity chromatography (IMAC) (5 mL HisTrap, Cytiva) and Superdex 200 26/60 (Cytiva) size-exclusion and complexed in vitro (1-3 hrs incubation at room temperature) prior to tag cleavage by recombinant his-tagged Tobacco Etch Virus protease or his-tagged Human rhinovirus 3C protease, as appropriate. The cleaved complex is further purified by Superdex 75 26/60 (Cytiva) size-exclusion followed by reverse-IMAC, in which the complex is in the column flow-through and residual His-MBP and his-tagged proteases are retained. The final complex is concentrated as desired by centrifugal concentration (Amicon Ultra 10,000 Da MWCO 15 mL concentrators, Millipore).

For E. coli expressed CDK2 and His-MBP-RB1[773-924]-c-avi, the proteins were biotinylated in vitro after the first IMAC step.¹

For CDK4/Cyclin D1 and CDK6/Cyclin D3, the complexes were expressed by simultaneous co-infection of insect cells by three separate viruses including one expressing untagged E. coli BirA. The expression media is supplemented with 50 uM d-biotin. The resulting protein complexes purified similarly. After lysis and clarification, the complex is purified by IMAC, Superdex 200 26/60 size exclusion, tag cleavage by human rhinovirus 3C protease, Superdex 75 26/60 size exclusion and reverse IMAC. BirA is typically not retained on the first IMAC, and excess CDK or Cyclin monomer is separated on the Superdex 200 size exclusion. Cleaved His-MBP and remaining monomer are removed by the Superdex 75 size exclusion and the reverse IMAC step. Final concentration by centrifugal concentrators as described above.

For the is-MBP-RB1[773-924]-c-avi, the protein was lysed by sonication and clarified by centrifugation. After IMAC capture and in vitro biotinylation, the fusion protein is purified by Q HP HiTrap (Cytiva) with a gradient elution after binding in low salt, followed by SP HP HiTrap (Cytiva) with a gradient elution. The material is finally purified on a Superdex 75 26/60 as a polishing step. Although there is a TEV protease cleavage site in this construct, it is not removed for this purpose. Final concentration by centrifugal concentrators as described above.

9x His-MBP-3C-CDK2-c-avi
(SEQ ID NO. 1)
MHHHHHHHHHMKIEEGKLVIWINGDKGYNGLAEVGKKFEKDTGIKVTVEHPDKLEEKFPQVAA
TGDGPDIIFWAHDRFGGYAQSGLLAEITPDKAFQDKLYPFTWDAVRYNGKLIAYPIAVEALSL
IYNKDLLPNPPKTWEEIPALDKELKAKGKSALMFNLQEPYFTWPLIAADGGYAFKYENGKYDI
KDVGVDNAGAKAGLTFLVDLIKNKHMNADTDYSIAEAAFNKGETAMTINGPWAWSNIDTSKVN
YGVTVLPTFKGQPSKPFVGVLSAGINAASPNKELAKEFLENYLLTDEGLEAVNKDKPLGAVAL
KSYEEELAKDPRIAATMENAQKGEIMPNIPQMSAFWYAVRTAVINAASGRQTVDEALKDAQTN
SSSGLEVLFQGPMENFQKVEKIGEGTYGVVYKARNKLTGEVVALKKIRLDTETEGVPSTAIRE
ISLLKELNHPNIVKLLDVIHTENKLYLVFEFLHQDLKKFMDASALTGIPLPLIKSYLFQLLQG
LAFCHSHRVLHRDLKPQNLLINTEGAIKLADFGLARAFGVPVRTYTHEVVTLWYRAPEILLGC
KYYSTAVDIWSLGCIFAEMVTRRALFPGDSEIDQLFRIFRTLGTPDEVVWPGVTSMPDYKPSF
PKWARQDFSKVVPPLDEDGRSLLSQMLHYDPNKRISAKAALAHPFFQDVTKPVPHLRLGGSGG
GGSGLNDIFEAQKIEWHE*
9x His-MBP-TEV-Cyclin E1
(SEQ ID NO. 2)
MHHHHHHHHHMKIEEGKLVIVINGDKGYNGLAEVGKKFEKDTGIKVTVEHPDKLEEKFPQVAA
TGDGPDIIFWAHDRFGGYAQSGLLAEITPDKAFQDKLYPFTWDAVRYNGKLIAYPIAVEALSL
IYNKDLLPNPPKTWEEIPALDKELKAKGKSALMFNLQEPYFTWPLIAADGGYAFKYENGKYDI
KDVGVDNAGAKAGLTFLVDLIKNKHMNADTDYSIAEAAFNKGETAMTINGPWAWSNIDTSKVN
YGVTVLPTFKGQPSKPFVGVLSAGINAASPNKELAKEFLENYLLTDEGLEAVNKDKPLGAVAL
KSYEEELAKDPRIAATMENAQKGEIMPNIPQMSAFWYAVRTAVINAASGRQTVDEALKDAQTN
SSSENLYFQGGSGMPRERRERDAKERDTMKEDGGAEFSARSRKRKANVTVFLQDPDEEMAKID
RTARDQCGSQPWDNNAVCADPCSLIPTPDKEDDDRVYPNSTCKPRIIAPSRGSPLPVLSWANR
EEVWKIMLNKEKTYLRDQHFLEQHPLLQPKMRAILLDWLMEVCEVYKLHRETFYLAQDFFDRY
MATQENVVKTLLQLIGISSLFIAAKLEEIYPPKLHQFAYVTDGACSGDEILTMELMIMKALKW
RLSPLTIVSWLNVYMQVAYLNDLHEVLLPQYPQQIFIQIAELLDLCVLDVDCLEFPYGILAAS
ALYHFSSSELMQKVSGYQWCDIENCVKWMVPFAMVIRETGSSKLKHFRGVADEDAHNIQTHRD
SLDLLDKARAKKAMLSEQNRASPLPSGLLTPPQSGKKQSSGPEMA*
9x His-MBP-TEV-CDK1-c-avi
(SEQ ID NO. 3)
MHHHHHHHHHMKIEEGKLVIWINGDKGYNGLAEVGKKFEKDTGIKVTVEHPDKLEEKFPQVAA
TGDGPDIIFWAHDRFGGYAQSGLLAEITPDKAFQDKLYPFTWDAVRYNGKLIAYPIAVEALSL
IYNKDLLPNPPKTWEEIPALDKELKAKGKSALMFNLQEPYFTWPLIAADGGYAFKYENGKYDI
KDVGVDNAGAKAGLTFLVDLIKNKHMNADTDYSIAEAAFNKGETAMTINGPWAWSNIDTSKVN
YGVTVLPTFKGQPSKPFVGVLSAGINAASPNKELAKEFLENYLLTDEGLEAVNKDKPLGAVAL
KSYEEELAKDPRIAATMENAQKGEIMPNIPQMSAFWYAVRTAVINAASGRQTVDEALKDAQTN
SSSENLYFQGGMEDYTKIEKIGEGTYGVVYKGRHKTTGQVVAMKKIRLESEEEGVPSTAIREI
SLLKELRHPNIVSLQDVLMQDSRLYLIFEFLSMDLKKYLDSIPPGQYMDSSLVKSYLYQILQG
IVFCHSRRVLHRDLKPQNLLIDDKGTIKLADFGLARAFGIPIRVYTHEVVTLWYRSPEVLLGS
ARYSTPVDIWSIGTIFAELATKKPLFHGDSEIDQLFRIFRALGTPNNEVWPEVESLQDYKNTF
PKWKPGSLASHVKNLDENGLDLLSKMLIYDPAKRISGKMALNHPYFNDLDNQIKKMGGSGGGG
SGLNDIFEAQKIEWHE*
9x His-MBP-Cyclin B1
(SEQ ID NO. 4)
MHHHHHHHHHMKIEEGKLVIWINGDKGYNGLAEVGKKFEKDTGIKVTVEHPDKLEEKFPQVAA
TGDGPDIIFWAHDRFGGYAQSGLLAEITPDKAFQDKLYPFTWDAVRYNGKLIAYPIAVEALSL
IYNKDLLPNPPKTWEEIPALDKELKAKGKSALMFNLQEPYFTWPLIAADGGYAFKYENGKYDI
KDVGVDNAGAKAGLTFLVDLIKNKHMNADTDYSIAEAAFNKGETAMTINGPWAWSNIDTSKVN
YGVTVLPTFKGQPSKPFVGVLSAGINAASPNKELAKEFLENYLLTDEGLEAVNKDKPLGAVAL
KSYEEELAKDPRIAATMENAQKGEIMPNIPQMSAFWYAVRTAVINAASGRQTVDEALKDAQTN
SSSENLYFQGGMALRVTRNSKINAENKAKINMAGAKRVPTAPAATSKPGLRPRTALGDIGNKV
SEQLQAKMPMKKEAKPSATGKVIDKKLPKPLEKVPMLVPVPVSEPVPEPEPEPEPEPVKEEKL
SPEPILVDTASPSPMETSGCAPAEEDLCQAFSDVILAVNDVDAEDGADPNLCSEYVKDIYAYL
RQLEEEQAVRPKYLLGREVTGNMRAILIDWLVQVQMKFRLLQETMYMTVSIIDRFMQNNCVPK
KMLQLVGVTAMFIASKYEEMYPPEIGDFAFVTDNTYTKHQIRQMEMKILRALNFGLGRPLPLH
FLRRASKIGEVDVEQHTLAKYLMELTMLDYDMVHFPPSQIAAGAFCLALKILDNGEWTPTLQH
YLSYTEESLLPVMQHLAKNVVMVNQGLTKHMTVKNKYATSKHAKISTLPQLNSALVQDLAKAV
AKV*
9x His-MBP-CDK4-c-avi
(SEQ ID NO. 5)
MHHHHHHHHHMKIEEGKLVIVINGDKGYNGLAEVGKKFEKDTGIKVTVEHPDKLEEKFPQVAA
TGDGPDIIFWAHDRFGGYAQSGLLAEITPDKAFQDKLYPFTWDAVRYNGKLIAYPIAVEALSL
IYNKDLLPNPPKTWEEIPALDKELKAKGKSALMFNLQEPYFTWPLIAADGGYAFKYENGKYDI
KDVGVDNAGAKAGLTFLVDLIKNKHMNADTDYSIAEAAFNKGETAMTINGPWAWSNIDTSKVN
YGVTVLPTFKGQPSKPFVGVLSAGINAASPNKELAKEFLENYLLTDEGLEAVNKDKPLGAVAL
KSYEEELAKDPRIAATMENAQKGEIMPNIPQMSAFWYAVRTAVINAASGRQTVDEALKDAQTN
SSSENLYFQGGSGMATSRYEPVAEIGVGAYGTVYKARDPHSGHFVALKSVRVPNGGGGGGGLP
ISTVREVALLRRLEAFEHPNVVRLMDVCATSRTDREIKVTLVFEHVDQDLRTYLDKAPPPGLP
AETIKDLMRQFLRGLDFLHANCIVHRDLKPENILVTSGGTVKLADFGLARIYSYQMALTPVVV
TLWYRAPEVLLQSTYATPVDMWSVGCIFAEMFRRKPLFCGNSEADQLGKIFDLIGLPPEDDWP
RDVSLPRGAFPPRGPRPVQSVVPEMEESGAQLLLEMLTFNPHKRISAFRALQHSYLHKDEGNP
EGGSGGGGSGLNDIFEAQKIEWHE*
9x His-MBP-Cyclin D1
(SEQ ID NO. 6)
MHHHHHHHHHMKIEEGKLVIWINGDKGYNGLAEVGKKFEKDTGIKVTVEHPDKLEEKFPQVAA
TGDGPDIIFWAHDRFGGYAQSGLLAEITPDKAFQDKLYPFTWDAVRYNGKLIAYPIAVEALSL
IYNKDLLPNPPKTWEEIPALDKELKAKGKSALMFNLQEPYFTWPLIAADGGYAFKYENGKYDI
KDVGVDNAGAKAGLTFLVDLIKNKHMNADTDYSIAEAAFNKGETAMTINGPWAWSNIDTSKVN
YGVTVLPTFKGQPSKPFVGVLSAGINAASPNKELAKEFLENYLLTDEGLEAVNKDKPLGAVAL
KSYEEELAKDPRIAATMENAQKGEIMPNIPQMSAFWYAVRTAVINAASGRQTVDEALKDAQTN
SSSENLYFQGGSGMEHQLLCCEVETIRRAYPDANLLNDRVLRAMLKAEETCAPSVSYFKCVQK
EVLPSMRKIVATWMLEVCEEQKCEEEVFPLAMNYLDRFLSLEPVKKSRLQLLGATCMFVASKM
KETIPLTAEKLCIYTDNSIRPEELLQMELLLVNKLKWNLAAMTPHDFIEHFLSKMPEAEENKQ
IIRKHAQTFVALCATDVKFISNPPSMVAAGSVVAAVQGLNLRSPNNFLSYYRLTRFLSRVIKC
DPDCLRACQEQIEALLESSLRQAQQNMDPKAAEEEEEEEEEVDLACTPTDVRDVDI*
9x His-MBP-CDK6-c-avi
(SEQ ID NO. 7)
MHHHHHHHHHMKIEEGKLVIWINGDKGYNGLAEVGKKFEKDTGIKVTVEHPDKLEEKFPQVAA
TGDGPDIIFWAHDRFGGYAQSGLLAEITPDKAFQDKLYPFTWDAVRYNGKLIAYPIAVEALSL
IYNKDLLPNPPKTWEEIPALDKELKAKGKSALMFNLQEPYFTWPLIAADGGYAFKYENGKYDI
KDVGVDNAGAKAGLTFLVDLIKNKHMNADTDYSIAEAAFNKGETAMTINGPWAWSNIDTSKVN
YGVTVLPTFKGQPSKPFVGVLSAGINAASPNKELAKEFLENYLLTDEGLEAVNKDKPLGAVAL
KSYEEELAKDPRIAATMENAQKGEIMPNIPQMSAFWYAVRTAVINAASGRQTVDEALKDAQTN
SSSMMGSSSSGLEVLFQGPMEKDGLCRADQQYECVAEIGEGAYGKVFKARDLKNGGRFVALKR
VRVQTGEEGMPLSTIREVAVLRHLETFEHPNVVRLFDVCTVSRTDRETKLTLVFEHVDQDLTT
YLDKVPEPGVPTETIKDMMFQLLRGLDFLHSHRVVHRDLKPQNILVTSSGQIKLADFGLARIY
SFQMALTSVVVTLWYRAPEVLLQSSYATPVDLWSVGCIFAEMFRRKPLFRGSSDVDQLGKILD
VIGLPGEEDWPRDVALPRQAFHSKSAQPIEKFVTDIDELGKDLLLKCLTFNPAKRISAYSALS
HPYFQDLERCKENLDSHLPPSQNTSELNTAGGSGGGGSGLNDIFEAQKIEWHE*
9x His-MBP-Cyclin D3
(SEQ ID NO. 8)
MHHHHHHHHHMKIEEGKLVIWINGDKGYNGLAEVGKKFEKDTGIKVTVEHPDKLEEKFPQVAA
TGDGPDIIFWAHDRFGGYAQSGLLAEITPDKAFQDKLYPFTWDAVRYNGKLIAYPIAVEALSL
IYNKDLLPNPPKTWEEIPALDKELKAKGKSALMFNLQEPYFTWPLIAADGGYAFKYENGKYDI
KDVGVDNAGAKAGLTFLVDLIKNKHMNADTDYSIAEAAFNKGETAMTINGPWAWSNIDTSKVN
YGVTVLPTFKGQPSKPFVGVLSAGINAASPNKELAKEFLENYLLTDEGLEAVNKDKPLGAVAL
KSYEEELAKDPRIAATMENAQKGEIMPNIPQMSAFWYAVRTAVINAASGRQTVDEALKDAQTN
SSSLEVLQGPGSMELLCCEGTRHAPRAGPDPRLLGDQRVLQSLLRLEERYVPRASYFQCVQRE
IKPHMRKMLAYWMLEVCEEQRCEEEVFPLAMNYLDRYLSCVPTRKAQLQLLGAVCMLLASKLR
ETTPLTIEKLCIYTDHAVSPRQLRDWEVLVLGKLKWDLAAVIAHDFLAFILHRLSLPRDRQAL
VKKHAQTFLALCATDYTFAMYPPSMIATGSIGAAVQGLGACSMSGDELTELLAGITGTEVDCL
RACQEQIEAALRESLREASQTSSSPAPKAPRGSSSQGPSQTSTPTDVTAIHL*
9x His-MBP- RB1[773-924]-c-avi
(SEQ ID NO. 9)
MHHHHHHHHHMKIEEGKLVIVINGDKGYNGLAEVGKKFEKDTGIKVTVEHPDKLEEKFPQVAA
TGDGPDIIFWAHDRFGGYAQSGLLAEITPDKAFQDKLYPFTWDAVRYNGKLIAYPIAVEALSL
IYNKDLLPNPPKTWEEIPALDKELKAKGKSALMFNLQEPYFTWPLIAADGGYAFKYENGKYDI
KDVGVDNAGAKAGLTFLVDLIKNKHMNADTDYSIAEAAFNKGETAMTINGPWAWSNIDTSKVN
YGVTVLPTFKGQPSKPFVGVLSAGINAASPNKELAKEFLENYLLTDEGLEAVNKDKPLGAVAL
KSYEEELAKDPRIAATMENAQKGEIMPNIPQMSAFWYAVRTAVINAASGRQTVDEALKDAQTN
SSSENLYFQGGSTRPPTLSPIPHIPRSPYKFPSSPLRIPGGNIYISPLKSPYKISEGLPTPTK
MTPRSRILVSIGESFGTSEKFQKINQMVCNSDRVLKRSAEGSNPPKPLKKLRFDIEGSDEADG
SKHLPGESKFQQKLAEMTSTRTRMQKQKMNDSMDTSNGGSGGGGSGLNDIFEAQKIEWHE*

1. Li, Y. & Sousa, R. Expression and purification of E. coli BirA biotin ligase for in vitro biotinylation. Protein Expr. Purif. 82, 162-7 (2012).
2. Gibson, D. G. et al. Enzymatic assembly of DNA molecules up to several hundred kilobases. Nat. Methods 6, 343-345 (2009).
3. Invitrogen. Bac-to-Bac® Baculovirus Expression System. An Efficient Site-Specific Transposition System to Generate Baculovirus for High-Level Expression of Recombinant Proteins. (2010).
4. Invitrogen life technologies. Guide to Baculovirus Expression Vector Systems (BEVS) and Insect Cell Culture Techniques. (2002).

IC₅₀ values determined in the biochemical assays are listed in the following table:

| | | CDK1 | CDK4 | CDK6 |
|---|---|---|---|---|
| | CDK2 | IC50 | IC50 | IC50 |
| Compound | IC50 (μM) | (μM) | (μM) | (μM) |
| 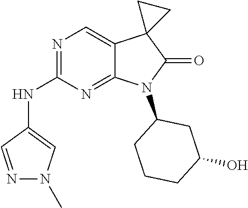 [SMILES: Cn1cc(Nc2ncc3c(n2)N([C@@H]2CCC[C@@H](O)C2)C(=O)C32CC2)cn1] | 0.038 | 1.54 | 0.40 | 0.60 |
| (Example 1) | | | | |
| 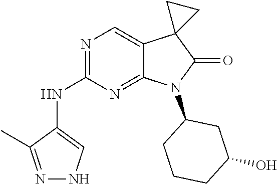 [SMILES: Cc1n[nH]cc1Nc1ncc2c(n1)N([C@@H]1CCC[C@@H](O)C1)C(=O)C21CC1] | 0.018 | 1.21 | 4.52 | 6.68 |
| (Example 2) | | | | |
| 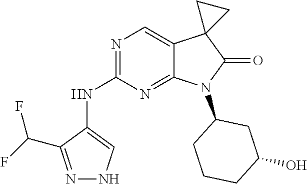 [SMILES: O=C1N([C@@H]2CCC[C@@H](O)C2)c2nc(Nc3c[nH]nc3C(F)F)ncc2C12CC2] | 0.007 | 0.82 | 2.74 | 3.76 |
| (Example 3) | | | | |
| 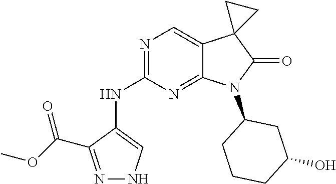 [SMILES: COC(=O)c1n[nH]cc1Nc1ncc2c(n1)N([C@@H]1CCC[C@@H](O)C1)C(=O)C21CC1] | 0.096 | ≥10.00 | ≥10.00 | ≥10.00 |
| (Example 4) | | | | |
| 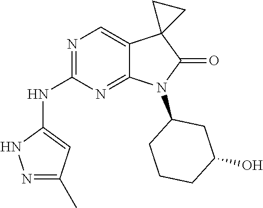 [SMILES: Cc1cc(Nc2ncc3c(n2)N([C@@H]2CCC[C@@H](O)C2)C(=O)C32CC2)[nH]n1] | 1.12 | ≥10.00 | 6.69 | 5.73 |
| (Example 5) | | | | |
| 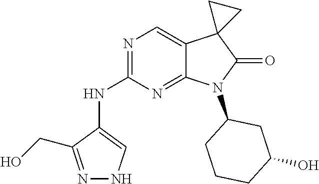 [SMILES: O=C1N([C@@H]2CCC[C@@H](O)C2)c2nc(Nc3c[nH]nc3CO)ncc2C12CC2] | 0.032 | 2.94 | 0.84 | 2.18 |
| (Example 6) | | | | |
| 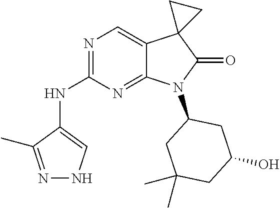 [SMILES: Cc1n[nH]cc1Nc1ncc2c(n1)N([C@H]1C[C@H](O)CC(C)(C)C1)C(=O)C21CC1] | 0.136 | 5.99 | 3.86 | 2.21 |
| (Example 7) | | | | |
| 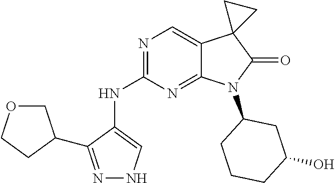 [SMILES: O=C1N([C@@H]2CCC[C@@H](O)C2)c2nc(Nc3c[nH]nc3C3CCOC3)ncc2C12CC2] | 0.234 | ≥10.00 | ≥10.00 | ≥10.00 |
| (Example 8) | | | | |
| 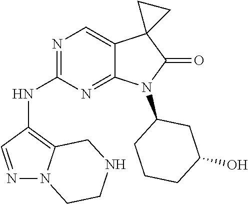 [SMILES: O=C1N([C@@H]2CCC[C@@H](O)C2)c2nc(Nc3cnn4c3CNCC4)ncc2C12CC2] | 0.268 | ≥10.00 | ≥10.00 | ≥10.00 |
| (Example 9) | | | | |
| 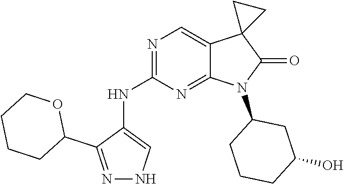 [SMILES: O=C1N([C@@H]2CCC[C@@H](O)C2)c2nc(Nc3c[nH]nc3C3CCCCO3)ncc2C12CC2] | 1.22 | ≥10.00 | ≥10.00 | ≥10.00 |
| (Example 10) | | | | |
| 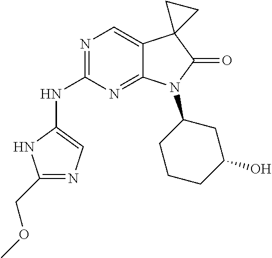 [SMILES: COCc1ncc(Nc2ncc3c(n2)N([C@@H]2CCC[C@@H](O)C2)C(=O)C32CC2)[nH]1] | 1.354 | ≥10.00 | 5.73 | 9.35 |
| (Example 11) | | | | |
| 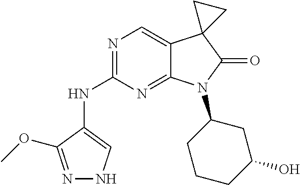 [SMILES: COc1n[nH]cc1Nc1ncc2c(n1)N([C@@H]1CCC[C@@H](O)C1)C(=O)C21CC1] | 0.020 | 2.24 | 7.37 | 7.90 |
| (Example 12) | | | | |
| 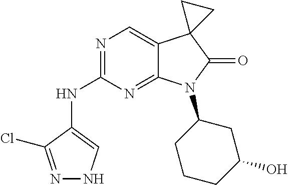 [SMILES: O=C1N([C@@H]2CCC[C@@H](O)C2)c2nc(Nc3c[nH]nc3Cl)ncc2C12CC2] | 0.012 | 0.92 | 5.28 | 7.10 |
| (Example 13) | | | | |
| 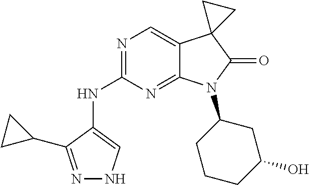 [SMILES: O=C1N([C@@H]2CCC[C@@H](O)C2)c2nc(Nc3c[nH]nc3C3CC3)ncc2C12CC2] | 0.030 | 1.55 | ≥10.00 | ≥10.00 |
| (Example 14) | | | | |
| 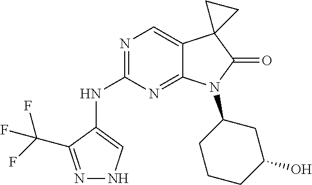 [SMILES: O=C1N([C@@H]2CCC[C@@H](O)C2)c2nc(Nc3c[nH]nc3C(F)(F)F)ncc2C12CC2] | 0.039 | 3.72 | ≥10.00 | ≥10.00 |
| (Example 15) | | | | |
| 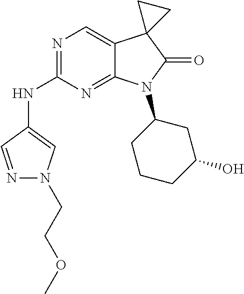 [SMILES: COCCn1cc(Nc2ncc3c(n2)N([C@@H]2CCC[C@@H](O)C2)C(=O)C32CC2)cn1] | 0.049 | 2.36 | 1.23 | 1.80 |
| (Example 16) | | | | |
| 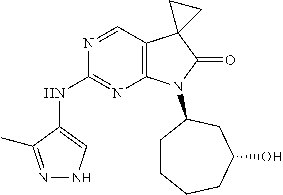 [SMILES: Cc1n[nH]cc1Nc1ncc2c(n1)N([C@@H]1CCCC[C@@H](O)C1)C(=O)C21CC1] | 0.041 | 4.49 | ≥10.00 | ≥10.00 |
| (Example 17) | | | | |
| 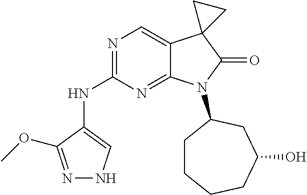 [SMILES: COc1n[nH]cc1Nc1ncc2c(n1)N([C@@H]1CCCC[C@@H](O)C1)C(=O)C21CC1] | 0.114 | ≥10.00 | ≥10.00 | Not tested |
| (Example 18) | | | | |
| 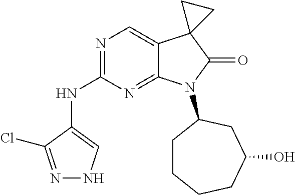 [SMILES: O=C1N([C@@H]2CCCC[C@@H](O)C2)c2nc(Nc3c[nH]nc3Cl)ncc2C12CC2] | 0.044 | 4.12 | 7.58 | 9.16 |
| (Example 19) | | | | |
| 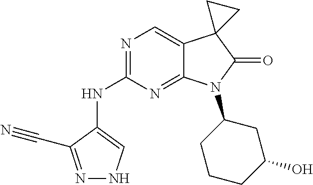 [SMILES: N#Cc1n[nH]cc1Nc1ncc2c(n1)N([C@@H]1CCC[C@@H](O)C1)C(=O)C21CC1] | 0.008 | 0.66 | 2.16 | 2.66 |
| (Example 20) | | | | |
| 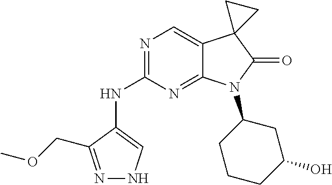 [SMILES: COCc1n[nH]cc1Nc1ncc2c(n1)N([C@@H]1CCC[C@@H](O)C1)C(=O)C21CC1] | 0.017 | 2.50 | 2.26 | 4.46 |
| (Example 21) | | | | |
| 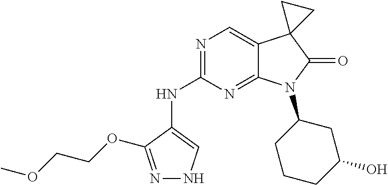 [SMILES: COCCOc1n[nH]cc1Nc1ncc2c(n1)N([C@@H]1CCC[C@@H](O)C1)C(=O)C21CC1] | 0.034 | 2.96 | 5.26 | 8.12 |
| (Example 22) | | | | |
| 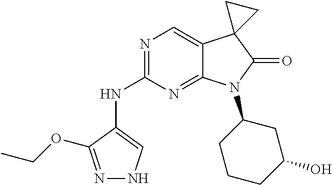 [SMILES: CCOc1n[nH]cc1Nc1ncc2c(n1)N([C@@H]1CCC[C@@H](O)C1)C(=O)C21CC1] | 0.015 | 2.43 | 9.45 | ≥10.00 |
| (Example 23) | | | | |
| 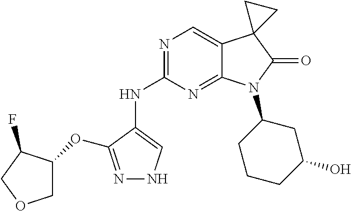 [SMILES: O=C1N([C@@H]2CCC[C@@H](O)C2)c2nc(Nc3c[nH]nc3O[C@@H]3COC[C@H]3F)ncc2C12CC2] | 0.012 | 2.02 | 3.91 | ≥10 |
| (Example 24) | | | | |
| 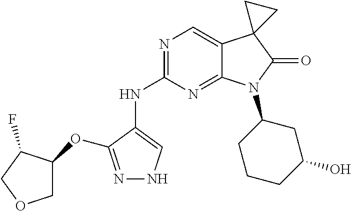 [SMILES: O=C1N([C@@H]2CCC[C@@H](O)C2)c2nc(Nc3c[nH]nc3O[C@H]3COC[C@@H]3F)ncc2C12CC2] | 0.024 | 2.44 | ≥10 | ≥10 |
| (Example 25) | | | | |
| 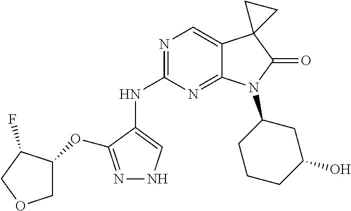 [SMILES: O=C1N([C@@H]2CCC[C@@H](O)C2)c2nc(Nc3c[nH]nc3O[C@@H]3COC[C@@H]3F)ncc2C12CC2] | 0.059 | ≥10 | 5.35 | 8.50 |
| (Example 26) | | | | |
| 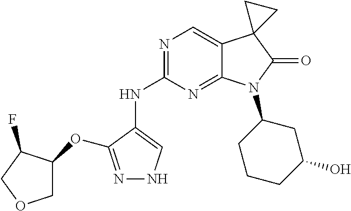 [SMILES: O=C1N([C@@H]2CCC[C@@H](O)C2)c2nc(Nc3c[nH]nc3O[C@H]3COC[C@H]3F)ncc2C12CC2] | 0.011 | 1.92 | 1.28 | 3.18 |
| (Example 27) | | | | |
| 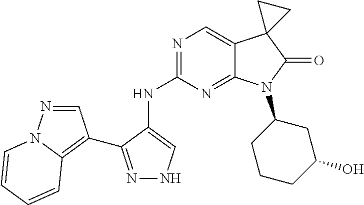 [SMILES: O=C1N([C@@H]2CCC[C@@H](O)C2)c2nc(Nc3c[nH]nc3-c3cnn4ccccc34)ncc2C12CC2] | 0.011 | 1.18 | 2.12 | 2.54 |
| (Example 28) | | | | |
| 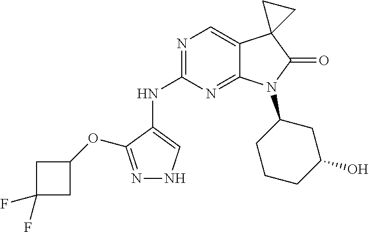 [SMILES: O=C1N([C@@H]2CCC[C@@H](O)C2)c2nc(Nc3c[nH]nc3OC3CC(F)(F)C3)ncc2C12CC2] | 0.026 | 3.84 | 7.40 | ≥10 |
| (Example 29) | | | | |
| 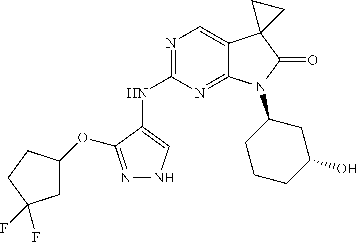 [SMILES: O=C1N([C@@H]2CCC[C@@H](O)C2)c2nc(Nc3c[nH]nc3OC3CCC(F)(F)C3)ncc2C12CC2] | 0.022 | 4.27 | 6.95 | 9.80 |
| Example 30 | | | | |
| 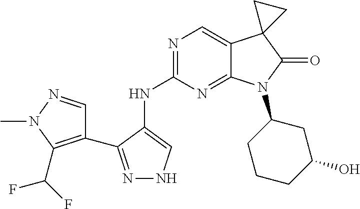 [SMILES: Cn1ncc(-c2n[nH]cc2Nc2ncc3c(n2)N([C@@H]2CCC[C@@H](O)C2)C(=O)C32CC2)c1C(F)F] | 0.021 | 6.57 | 6.30 | 9.70 |
| (Example 31) | | | | |
| 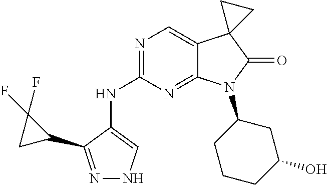 | 0.006 | 0.34 | 2.76 | 4.80 |
| (Example 32) | | | | |
| 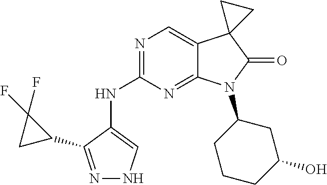 | 0.049 | 3.76 | ≥10 | ≥10 |
| (Example 33) | | | | |
| 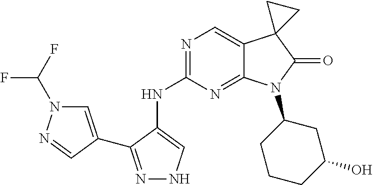 [SMILES: O=C1N([C@@H]2CCC[C@@H](O)C2)c2nc(Nc3c[nH]nc3-c3cnn(C(F)F)c3)ncc2C12CC2] | 0.010 | 2.31 | 3.31 | 3.31 |
| (Example 34) | | | | |
| 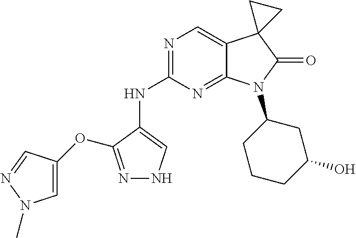 [SMILES: Cn1cc(Oc2n[nH]cc2Nc2ncc3c(n2)N([C@@H]2CCC[C@@H](O)C2)C(=O)C32CC2)cn1] | 0.054 | 3.16 | ≥10 | ≥10 |
| (Example 35) | | | | |
| 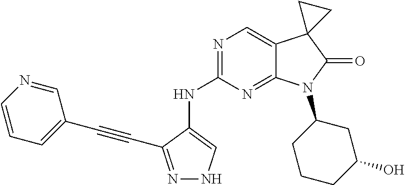 [SMILES: O=C1N([C@@H]2CCC[C@@H](O)C2)c2nc(Nc3c[nH]nc3C#Cc3cccnc3)ncc2C12CC2] | 0.039 | 4.99 | ≥10 | ≥10 |
| (Example 36) | | | | |
| 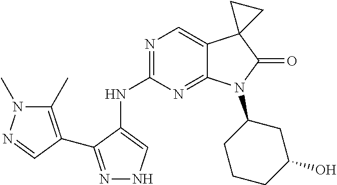 [SMILES: Cc1c(-c2n[nH]cc2Nc2ncc3c(n2)N([C@@H]2CCC[C@@H](O)C2)C(=O)C32CC2)cnn1C] | 0.031 | ≥10 | ≥10 | ≥10 |
| (Example 37) | | | | |
| 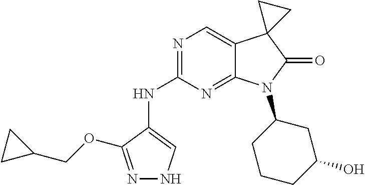 [SMILES: O=C1N([C@@H]2CCC[C@@H](O)C2)c2nc(Nc3c[nH]nc3OCC3CC3)ncc2C12CC2] | 0.008 | 1.04 | 6.10 | 9.20 |
| (Example 38) | | | | |
| 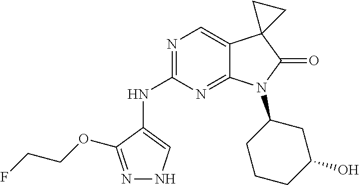 [SMILES: O=C1N([C@@H]2CCC[C@@H](O)C2)c2nc(Nc3c[nH]nc3OCCF)ncc2C12CC2] | 0.007 | 1.20 | 1.43 | 2.95 |
| (Example 39) | | | | |
| 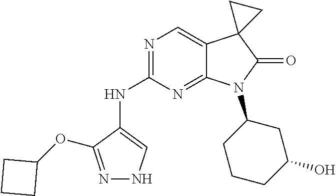 [SMILES: O=C1N([C@@H]2CCC[C@@H](O)C2)c2nc(Nc3c[nH]nc3OC3CCC3)ncc2C12CC2] | 0.026 | 2.89 | ≥10 | ≥10 |
| (Example 40) | | | | |
| 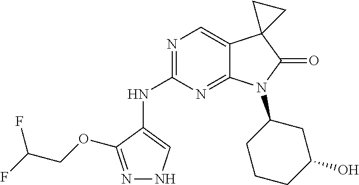 [SMILES: O=C1N([C@@H]2CCC[C@@H](O)C2)c2nc(Nc3c[nH]nc3OCC(F)F)ncc2C12CC2] | 0.005 | 0.73 | 2.68 | 5.09 |
| (Example 41) | | | | |
| 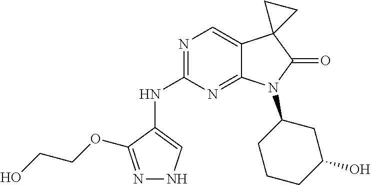 [SMILES: O=C1N([C@@H]2CCC[C@@H](O)C2)c2nc(Nc3c[nH]nc3OCCO)ncc2C12CC2] | 0.024 | 3.74 | 3.64 | 3.01 |
| (Example 42) | | | | |
| 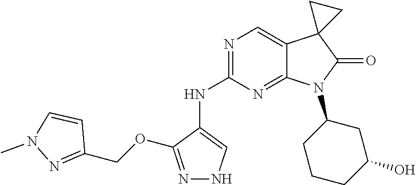 [SMILES: Cn1ccc(COc2n[nH]cc2Nc2ncc3c(n2)N([C@@H]2CCC[C@@H](O)C2)C(=O)C32CC2)n1] | 0.019 | 1.76 | 3.09 | 8.84 |
| (Example 43) | | | | |
| 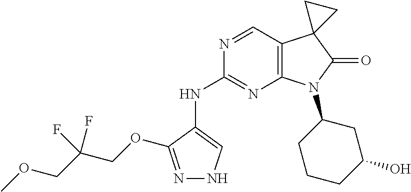 [SMILES: COCC(F)(F)COc1n[nH]cc1Nc1ncc2c(n1)N([C@@H]1CCC[C@@H](O)C1)C(=O)C21CC1] | 0.015 | 1.81 | 4.41 | 7.13 |
| (Example 44) | | | | |
| 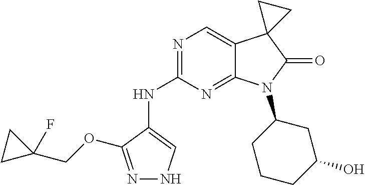 [SMILES: O=C1N([C@@H]2CCC[C@@H](O)C2)c2nc(Nc3c[nH]nc3OCC3(F)CC3)ncc2C12CC2] | 0.011 | 0.77 | 1.23 | 3.50 |
| (Example 45) | | | | |
| 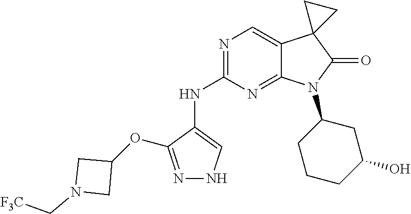 [SMILES: O=C1N([C@@H]2CCC[C@@H](O)C2)c2nc(Nc3c[nH]nc3OC3CN(CC(F)(F)F)C3)ncc2C12CC2] | 0.109 | ≥10 | ≥10 | ≥10 |
| (Example 46) | | | | |
| 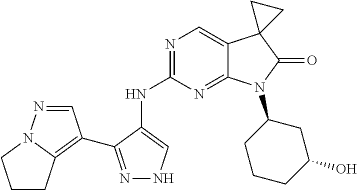 [SMILES: O=C1N([C@@H]2CCC[C@@H](O)C2)c2nc(Nc3c[nH]nc3-c3cnn4c3CCC4)ncc2C12CC2] | 0.027 | ≥10 | 6.38 | 7.55 |
| (Example 47) | | | | |
| 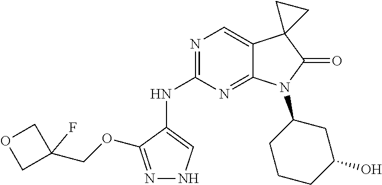 [SMILES: O=C1N([C@@H]2CCC[C@@H](O)C2)c2nc(Nc3c[nH]nc3OCC3(F)COC3)ncc2C12CC2] | 0.022 | 3.25 | 1.54 | ≥10 |
| (Example 48) | | | | |
| 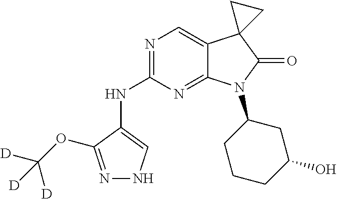 [SMILES: [2H]C([2H])([2H])Oc1n[nH]cc1Nc1ncc2c(n1)N([C@@H]1CCC[C@@H](O)C1)C(=O)C21CC1] | 0.015 | 2.64 | 9.77 | ≥10 |
| (Example 49) | | | | |
| 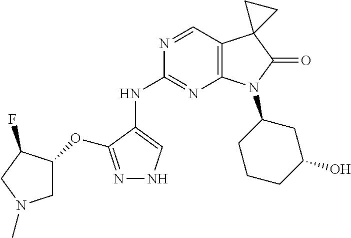 [SMILES: CN1C[C@@H](F)[C@H](Oc2n[nH]cc2Nc2ncc3c(n2)N([C@@H]2CCC[C@@H](O)C2)C(=O)C32CC2)C1] | 0.125 | ≥10 | ≥10 | ≥10 |
| (Example 50) | | | | |
| 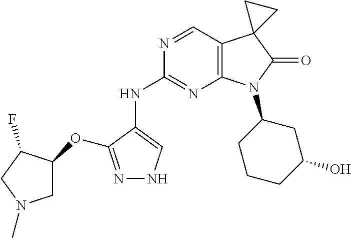 [SMILES: CN1C[C@H](Oc2n[nH]cc2Nc2ncc3c(n2)N([C@@H]2CCC[C@@H](O)C2)C(=O)C32CC2)[C@@H](F)C1] | 0.119 | ≥10 | ≥10 | ≥10 |
| (Example 51) | | | | |
| 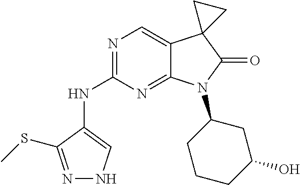 [SMILES: CSc1n[nH]cc1Nc1ncc2c(n1)N([C@@H]1CCC[C@@H](O)C1)C(=O)C21CC1] | 0.031 | 5.32 | not tested | ≥10 |
| (Example 52) | | | | |
| 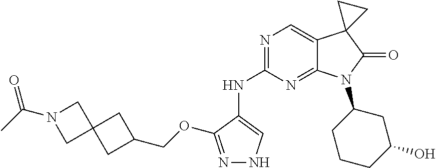 [SMILES: CC(=O)N1CC2(CC(COc3n[nH]cc3Nc3ncc4c(n3)N([C@@H]3CCC[C@@H](O)C3)C(=O)C43CC3)C2)C1] | 0.186 | ≥10 | ≥10 | ≥10 |
| (Example 53) | | | | |
| 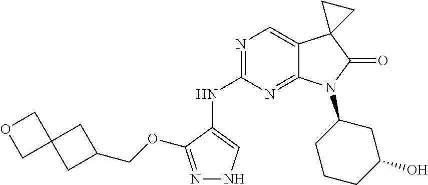 [SMILES: O=C1N([C@@H]2CCC[C@@H](O)C2)c2nc(Nc3c[nH]nc3OCC3CC4(COC4)C3)ncc2C12CC2] | 0.134 | ≥10 | ≥10 | ≥10 |
| (Example 54) | | | | |
| 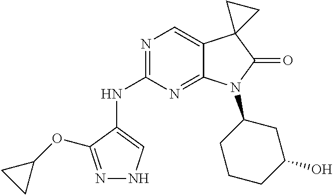 [SMILES: O=C1N([C@@H]2CCC[C@@H](O)C2)c2nc(Nc3c[nH]nc3OC3CC3)ncc2C12CC2] | 0.011 | 1.43 | 8.50 | ≥10 |
| (Example 55) | | | | |
| 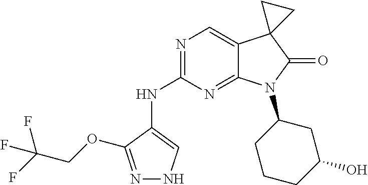 [SMILES: O=C1N([C@@H]2CCC[C@@H](O)C2)c2nc(Nc3c[nH]nc3OCC(F)(F)F)ncc2C12CC2] | 0.010 | 0.80 | 3.13 | 6.28 |
| (Example 56) | | | | |
| 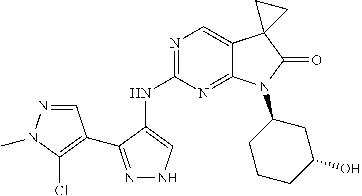 [SMILES: Cn1ncc(-c2n[nH]cc2Nc2ncc3c(n2)N([C@@H]2CCC[C@@H](O)C2)C(=O)C32CC2)c1Cl] | 0.015 | 2.04 | 7.90 | ≥10 |
| (Example 57) | | | | |
| 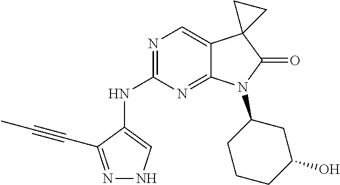 [SMILES: CC#Cc1n[nH]cc1Nc1ncc2c(n1)N([C@@H]1CCC[C@@H](O)C1)C(=O)C21CC1] | 0.016 | 3.54 | 9.50 | ≥10 |
| (Example 58) | | | | |
| 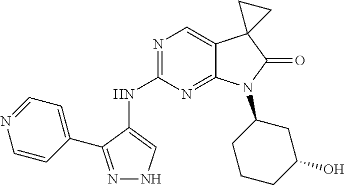 [SMILES: O=C1N([C@@H]2CCC[C@@H](O)C2)c2nc(Nc3c[nH]nc3-c3ccncc3)ncc2C12CC2] | 0.251 | ≥10 | ≥10 | ≥10 |
| (Example 59) | | | | |
| 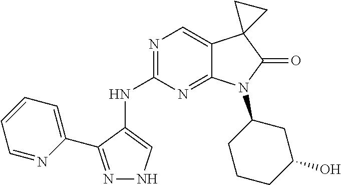 [SMILES: O=C1N([C@@H]2CCC[C@@H](O)C2)c2nc(Nc3c[nH]nc3-c3ccccn3)ncc2C12CC2] | 0.162 | ≥10 | ≥10 | ≥10 |
| (Example 60) | | | | |
| 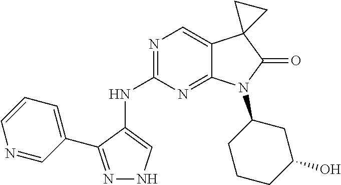 [SMILES: O=C1N([C@@H]2CCC[C@@H](O)C2)c2nc(Nc3c[nH]nc3-c3cccnc3)ncc2C12CC2] | 0.046 | ≥10 | 8.65 | 9.19 |
| (Example 61) | | | | |
| 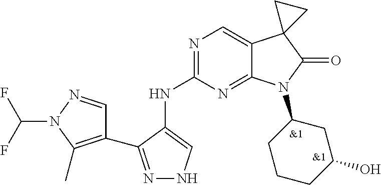 [SMILES: Cc1c(-c2n[nH]cc2Nc2ncc3c(n2)N([C@@H]2CCC[C@@H](O)C2)C(=O)C32CC2)cnn1C(F)F] | 0.017 | 4.23 | 2.30 | 1.83 |
| (Example 62) | | | | |
| 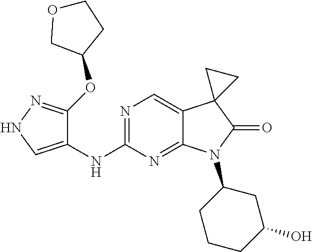 [SMILES: O=C1N([C@@H]2CCC[C@@H](O)C2)c2nc(Nc3c[nH]nc3O[C@@H]3CCOC3)ncc2C12CC2] | 0.043 | 3.69 | ≥10 | ≥10 |
| (Example 63) | | | | |
| 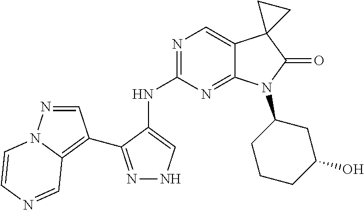 [SMILES: O=C1N([C@@H]2CCC[C@@H](O)C2)c2nc(Nc3c[nH]nc3-c3cnn4ccncc34)ncc2C12CC2] | 0.012 | 2.19 | 4.70 | 6.49 |
| (Example 64) | | | | |
| 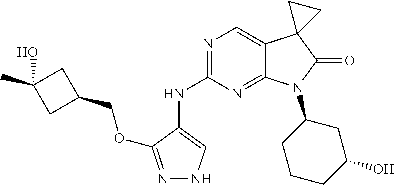 [SMILES: C[C@]1(O)C[C@H](COc2n[nH]cc2Nc2ncc3c(n2)N([C@@H]2CCC[C@@H](O)C2)C(=O)C32CC2)C1] | 0.053 | 4.49 | 6.90 | ≥10 |
| (Example 65) | | | | |
| 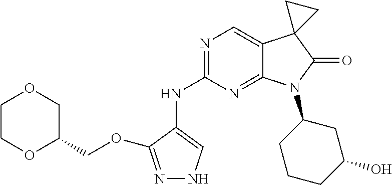 [SMILES: O=C1N([C@@H]2CCC[C@@H](O)C2)c2nc(Nc3c[nH]nc3OC[C@H]3COCCO3)ncc2C12CC2] | 0.051 | 4.22 | ≥10 | ≥10 |
| (Example 66) | | | | |
| 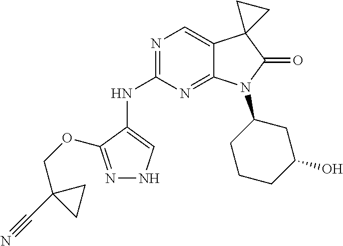 [SMILES: N#CC1(COc2n[nH]cc2Nc2ncc3c(n2)N([C@@H]2CCC[C@@H](O)C2)C(=O)C32CC2)CC1] | 0.023 | 4.13 | 6.91 | ≥10 |
| (Example 67) | | | | |
| 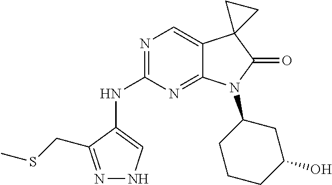 [SMILES: CSCc1n[nH]cc1Nc1ncc2c(n1)N([C@@H]1CCC[C@@H](O)C1)C(=O)C21CC1] | 0.007 | 0.97 | 4.15 | 7.60 |
| (Example 68) | | | | |
| 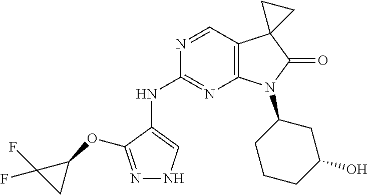 [SMILES: O=C1N([C@@H]2CCC[C@@H](O)C2)c2nc(Nc3c[nH]nc3O[C@H]3CC3(F)F)ncc2C12CC2] | 0.006 | 0.80 | 2.61 | 6.03 |
| (Example 69) | | | | |
| 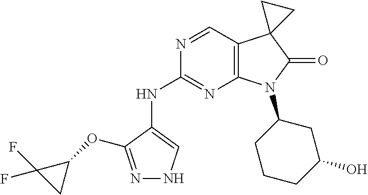 [SMILES: O=C1N([C@@H]2CCC[C@@H](O)C2)c2nc(Nc3c[nH]nc3O[C@@H]3CC3(F)F)ncc2C12CC2] | 0.006 | 0.82 | 7.69 | ≥10 |
| (Example 70) | | | | |
| 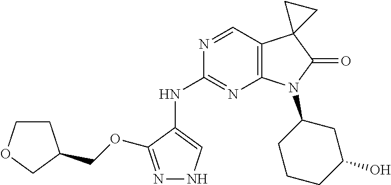 [SMILES: O=C1N([C@@H]2CCC[C@@H](O)C2)c2nc(Nc3c[nH]nc3OC[C@@H]3CCOC3)ncc2C12CC2] | 0.044 | 7.59 | ≥10 | ≥10 |
| (Example 71) | | | | |
| 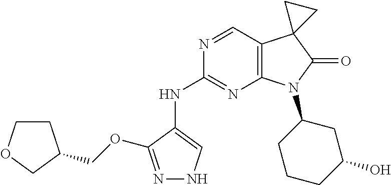 [SMILES: O=C1N([C@@H]2CCC[C@@H](O)C2)c2nc(Nc3c[nH]nc3OC[C@H]3CCOC3)ncc2C12CC2] | 0.061 | 7.50 | ≥10 | ≥10 |
| (Example 72) | | | | |
| 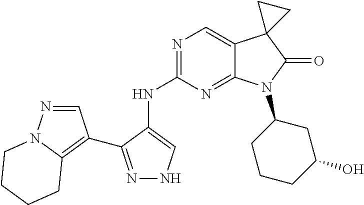 [SMILES: O=C1N([C@@H]2CCC[C@@H](O)C2)c2nc(Nc3c[nH]nc3-c3cnn4c3CCCC4)ncc2C12CC2] | 0.049 | ≥10 | ≥10 | ≥10 |
| (Example 73) | | | | |
| 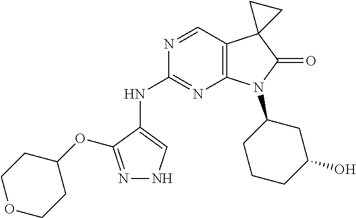 [SMILES: O=C1N([C@@H]2CCC[C@@H](O)C2)c2nc(Nc3c[nH]nc3OC3CCOCC3)ncc2C12CC2] | 0.255 | ≥10 | ≥10 | ≥10 |
| (Example 74) | | | | |
| 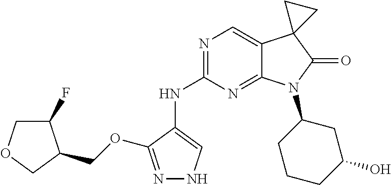 [SMILES: O=C1N([C@@H]2CCC[C@@H](O)C2)c2nc(Nc3c[nH]nc3OC[C@H]3COC[C@H]3F)ncc2C12CC2] | 0.072 | 9.16 | ≥10 | ≥10 |
| (Example 75) | | | | |
| 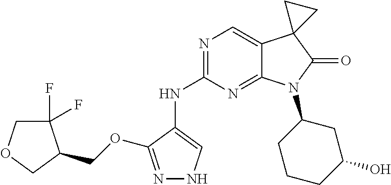 [SMILES: O=C1N([C@@H]2CCC[C@@H](O)C2)c2nc(Nc3c[nH]nc3OC[C@H]3COCC3(F)F)ncc2C12CC2] | 0.032 | 3.29 | 7.27 | ≥10 |
| (Example 76) | | | | |
| 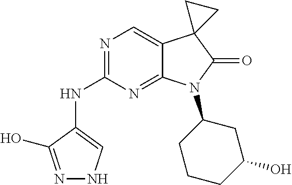 [SMILES: O=C1N([C@@H]2CCC[C@@H](O)C2)c2nc(Nc3c[nH]nc3O)ncc2C12CC2] | 0.121 | 5.82 | 1.48 | 5.26 |
| (Example 77) | | | | |
| 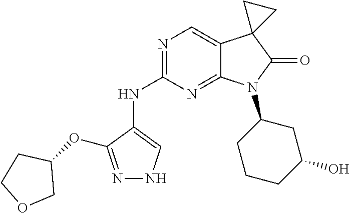 [SMILES: O=C1N([C@@H]2CCC[C@@H](O)C2)c2nc(Nc3c[nH]nc3O[C@H]3CCOC3)ncc2C12CC2] | 0.017 | 2.54 | 2.60 | 3.54 |
| (Example 78) | | | | |
| 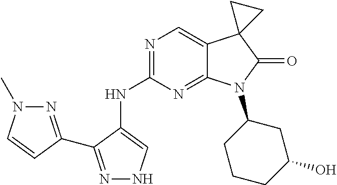 [SMILES: Cn1ccc(-c2n[nH]cc2Nc2ncc3c(n2)N([C@@H]2CCC[C@@H](O)C2)C(=O)C32CC2)n1] | 0.050 | ≥10 | ≥10 | ≥10 |
| (Example 79) | | | | |
| 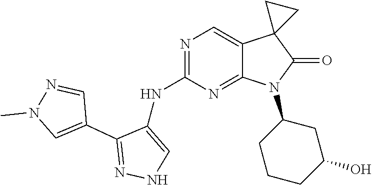 [SMILES: Cn1cc(-c2n[nH]cc2Nc2ncc3c(n2)N([C@@H]2CCC[C@@H](O)C2)C(=O)C32CC2)cn1] | 0.029 | 6.01 | 9.30 | ≥10 |
| (Example 80) | | | | |
| 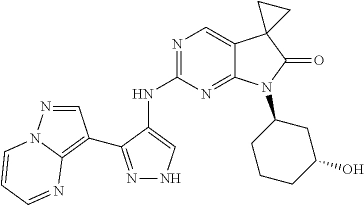 [SMILES: O=C1N([C@@H]2CCC[C@@H](O)C2)c2nc(Nc3c[nH]nc3-c3cnn4cccnc34)ncc2C12CC2] | 0.006 | 0.41 | ≥10 | ≥10 |
| (Example 81) | | | | |
| 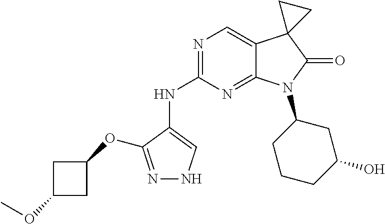 [SMILES: CO[C@H]1C[C@H](Oc2n[nH]cc2Nc2ncc3c(n2)N([C@@H]2CCC[C@@H](O)C2)C(=O)C32CC2)C1] | 0.026 | 2.53 | ≥10 | ≥10 |
| (Example 82) | | | | |
| 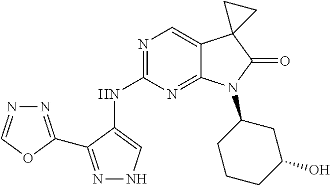 [SMILES: O=C1N([C@@H]2CCC[C@@H](O)C2)c2nc(Nc3c[nH]nc3-c3nnco3)ncc2C12CC2] | 0.031 | 8.62 | 2.18 | 2.67 |
| (Example 83) | | | | |
| 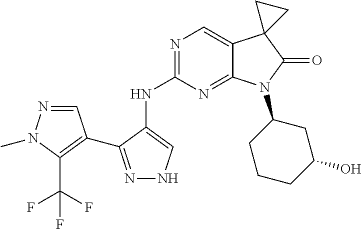 [SMILES: Cn1ncc(-c2n[nH]cc2Nc2ncc3c(n2)N([C@@H]2CCC[C@@H](O)C2)C(=O)C32CC2)c1C(F)(F)F] | 0.088 | ≥10 | ≥10 | ≥10 |
| (Example 84) | | | | |
| 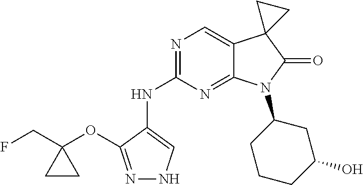 [SMILES: O=C1N([C@@H]2CCC[C@@H](O)C2)c2nc(Nc3c[nH]nc3OC3(CF)CC3)ncc2C12CC2] | 0.027 | 1.71 | 4.40 | ≥10 |
| (Example 85) | | | | |

### In Vivo Pharmacodynamic Assessment of CDK2 Inhibitors in Cyclin E Amplified OVCAR3 Xenograft Model

Cyclin-dependent kinase 2 (CDK2) is a member of the protein kinase family. It plays an important role in regulating various events of the eukaryotic cell division cycle. CDK2 is involved in the inactivation of the retinoblastoma protein (RB) by phosphorylation which then allows for the G1 to S phase transition of the cell cycle. Overexpression of cyclin E, the obligate binding partner of CDK2, can cause abnormal regulation of the cell cycle, which is directly associated with hyperproliferation of cancer cells. Therefore, CDK2 is regarded as a potentially impactful therapeutic target for cancer treatment. The purpose of these studies was to evaluate the PK/PD relationship of CDK2 inhibitors in Cyclin E amplified cancer cell lines in vivo in immunodeficient mice. Plasma and tumor tissues were collected for PK analysis and PD assays (read out of RB phosphorylation inhibition) after 3 days of dosing of the CDK2 inhibitor.

### Tumor Cell Inoculation and Treatments

The Cyclin E amplified ovarian cancer cell line OVCAR3 was tested to be free of Mycoplasma and murine viruses using the IDEXX Impact 8 panel. Cells were cultured in RPMI 1640 media supplemented with 20% FBS for fewer than 5 passages after initial thawing and prior to cell implantation into immunodeficient mice. Cells were harvested and resuspended in sterile PBS containing 50% Matrigel. Ten million viable cells were injected at a volume of 0.2 ml per mouse into the flank of immunocompromised NSG mice (1 implant per animal). When tumors reached a mean volume of 200-300 mm³, mice were randomized and treatments were initiated. Compounds were formulated in 10% HPBCD (hydroxypropyl beta cyclodextrin) and administered orally, twice a day, in a volume of 10 ml/kg. The dosing volume was adjusted based on individual mouse body weight on the day of dosing. Pharmacodynamic markers were evaluated on day 3.

### PK Analysis of Plasma

For determination of compound plasma concentrations, serial blood collections were performed at designated timepoints after dosing. Blood was collected in EDTA-lined tubes and placed on wet ice. Within 30 minutes of collection, blood was centrifuged, and the plasma supernatants were aliquoted and stored at −20 C until analyzed. The frozen plasma samples and tumor tissue were processed UPLC and LC-MS/MS analysis by the addition of organic solvent and spiked with the bioanalytical internal standards. CDK2 inhibitor concentrations were measured by LC-MS/MS analysis on API 4500, API 5500+, or Sciex 7500 instruments. UPLC separation was done using a kinetex evo C₁₈ column (50×4.6 mm, 5.0 μm) or a Waters Xbridge BEH C18 column (50×2.1 mm, 2.5 μm). The various analytes were detected using the mass transitions shown in the table below.

| | | Parent | Daughter | Collision |
|---|---|---|---|---|
| Compound | Type | [m/z] | [m/z] | energy |
| Compound 1 | Analyte | 371.3 | 273.2 | 25 |
| Compound 2 | Analyte | 386.2 | 259.1 | 42 |
| Compound 3 | Analyte | 454.2 | 336.0 | 44 |
| Telmisartan | Internal | 515.4 | 276.1 | 60 |
| | standard | | | |
| Glyburide | Internal | 494.2 | 169.2 | 35 |
| | standard | | | |

For the purposes of this Example (and for corresponding FIG. 3):
Compound 1=

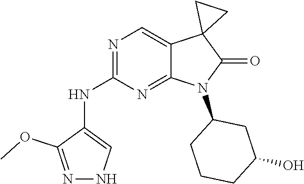 [SMILES: COc1n[nH]cc1Nc1ncc2c(n1)N([C@@H]1CCC[C@@H](O)C1)C(=O)C21CC1]

Compound 2=

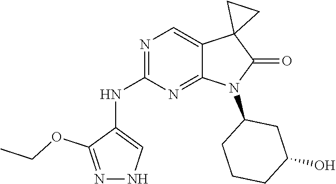 [SMILES: CCOc1n[nH]cc1Nc1ncc2c(n1)N([C@@H]1CCC[C@@H](O)C1)C(=O)C21CC1]

Compound 3=

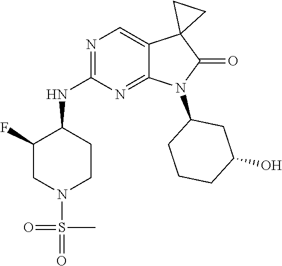 [SMILES: CS(=O)(=O)N1CC[C@H](Nc2ncc3c(n2)N([C@@H]2CCC[C@@H](O)C2)C(=O)C32CC2)[C@H](F)C1]

### PD Analysis of Tumor Samples

For terminal PD biomarker analysis, mice were euthanized following 3 days of treatment (1 hour post 5^{th} dose). Tumors were immediately excised and then sectioned into fragments. Tissue fragments were snap frozen in liquid nitrogen and stored at −80 C until processing for PD biomarker analysis. Tumors were lysed with the Precellys instrument using protein lysis buffer supplemented with a phosphatase and protease inhibitor cocktail. RB phosphorylation levels of the resulting tumor protein lysates were then determined by MSD. Ten microgram protein was loaded per well and total Rb and phosphorylated RB (Ser 807/811) capture and detection antibodies were used to evaluate the phosphorylated RB signal. Values were calculated and normalized to the untreated controls. Three mice per treatment arm were used.

### Results

The CDK2 inhibitors Compound 1 and Compound 2, dosed at 30 mg/kg twice daily (BID), result in ˜80% inhibition of RB phosphorylation at 1 hour after the last dose. In contrast, Compound 3, dosed at 60 mg/kg twice daily (BID), only results in ˜50% inhibition of RB phosphorylation at that same timepoint. For each compound, plasma concentration versus time profiles was assessed on day 1 and at a single time point (1 hour) following the administration of the third dose (49 h post the initial dosing). An estimate for the tumor concentration of the various analytes was also obtained at the 49 h time-point following the homogenization of the tissue.

### DISCUSSION

This PK/PD study in the Cyclin E amplified OVCAR3 xenograft model demonstrates that Compound 1 and Compound 2 (dosed at 30 mg/kg, BID) result in deeper inhibition of RB phosphorylation than Compound 3 dose twice as high (60 mg/kg, BID). The lower exposure observed with Compound 3 may be the result of reduced oral bioavailability at the administered dose (60 mpk) combined with higher systemic clearance compared to Compound 1 and Compound 2. In conclusion, Compound 1 and Compound 2 can be considered more potent CDK2 inhibitors than Compound 3.

1. A compound according to formula (I), 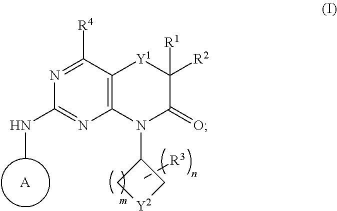
wherein:
Y¹ is a bond or CH₂;
Y² is a bond, O, NR⁵ or CR⁶R⁷;
R¹ and R² are each independently selected from the group consisting of H, halo, C₁-C₆alkyl and C₁-C₆haloalkyl, or R¹ and R² join together to form C₃-C₄cycloalkyl or C₃-C₄cyclohaloalkyl;
each R³ is independently selected from the group consisting of hydroxyl, halo, C₁-C₆alkyl and C₁-C₆haloalkyl;
R⁴ is selected from the group consisting of H, halo, C₁-C₆alkyl and C₁-C₆haloalkyl;
R⁵ is selected from the group consisting of H, C₁-C₆alkyl, C(═O)—C₁-C₆alkyl or C(═O)—O—C₁-C₆alkyl;
R⁶ and R⁷ join together to form, together with the carbon atom to which they are mutually attached, a C₃-C₆cycloalkyl or a 3-6 membered heterocyclyl comprising 1-3 heteroatoms independently selected from the group consisting of O, N and S, wherein said C₃-C₆cycloalkyl or 3-6 membered heterocyclyl is substituted with 0-3 substituents R⁸;
each R⁸ is independently selected from the group consisting of C₁-C₆alkyl, C(═O)C₁-C₆alkyl, halo, C₁-C₆haloalkyl, S—C₁-C₆alkyl, SO—C₁-C₆alkyl, SO₂—C₁-C₆alkyl, cyano, hydroxyl, or wherein two R⁸ substituents on the same ring atom join together to form ═O;
n is 0 to 3;
m is 1 to 5; 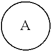
is a 5 membered heteroaryl comprising 1 to 3 heteroatoms independently selected from N, O and S, said 5 membered heteroaryl being substituted with 0 to 3 substituents R^{A};
each R^{A} is independently *L¹-X¹, wherein * indicates the point of attachment to 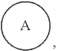
each L¹ is independently selected from bond, O, S, SO, SO₂, C≡C, C(═O), *C(═O)—O**, C₁-C₆alkylene, C₁-C₆haloalkylene, *O—C₁-C₆alkylene**, *O—C₁-C₆haloalkylene**, *O—C₁-C₆hydroxyalkylene**, C₁-C₆alkylene-O—C₁-C₆alkylene, *O—C₃-C₆cycloalkylene**, *O-3-6 membered heterocyclylene**, C₁-C₆hydroxyalkylene, C₃-C₆cycloalkylene, 3-6 membered heterocyclylene, O—C₁-C₆alkylene-O, *O—C₁-C₆alkylene-O—C₃-C₆cycloalkylene** and *O—C₁-C₆alkylene-O-3-6 membered heterocyclylene**, wherein * indicates the point of attachment to 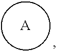
and ** indicates the point of attachment to X¹;
and each X¹ is independently selected from H, halo, cyano, hydroxyl, C₁-C₆alkyl, C(═O)—C₁-C₆alkyl, C₁-C₆haloalkyl, C₃-C₆cycloalkyl substituted by 0-3 R⁸ groups, O—C₁-C₆alkyl, S—C₁-C₆alkyl, S(O)—C₁-C₆alkyl, S(O)₂—C₁-C₆alkyl, N(C₁-C₆alkyl)₂, C(═O)N(C₁-C₆alkyl)₂, C₁-C₆hydroxyalkyl, 3-6 membered heterocyclyl substituted by 0-3 R⁸ groups, 5-10 membered heteroaryl comprising 1-4 heteroatoms independently selected from O, N and S substituted by 0-3 R⁸ groups, 5-10 membered partially saturated heterocyclyl comprising 1 to 4 heteroatoms independently selected from the group consisting of O, N and S substituted by 0-3 R⁸ groups, 7-10 membered spiroheterocyclyl comprising 1 or 2 heteroatoms independently selected from O, N and S substituted by 0-3 R⁸ groups and C₇-C₁₀spirocycloalkyl substituted by 0-3 R⁸ groups;
or
two R^{A} substituents located on adjacent ring atoms join together to form with said adjacent ring atoms a 4 to 6 membered heterocyclyl comprising 1 to 3 heteroatoms independently selected from N, O and S, with the proviso that at least one heteroatom is N;
or a pharmaceutically acceptable salt and/or tautomer thereof.

2. The compound or pharmaceutically acceptable salt and/or tautomer thereof according to claim 1, wherein Y¹ is a bond.

3. The compound or pharmaceutically acceptable salt and/or tautomer thereof according to claim 1, wherein Y² is a bond.

4. The compound or pharmaceutically acceptable salt and/or tautomer thereof according to claim 1, wherein m is 4.

5. The compound or pharmaceutically acceptable salt and/or tautomer thereof according to claim 1, wherein n is 1 to 3.

6. The compound or pharmaceutically acceptable salt and/or tautomer thereof according to claim 5, wherein at least one R³ is OH.

7. The compound or pharmaceutically acceptable salt and/or tautomer thereof according to claim 1, wherein the compound of formula (I) is a compound of formula (Ia): 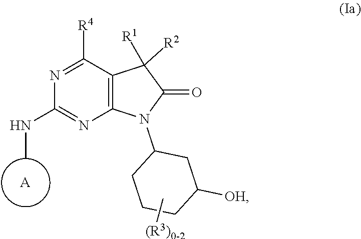
wherein R¹, R², R³, R⁴ and 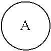
are as defined in claim 1.

8. The compound or pharmaceutically acceptable salt and/or tautomer thereof according to claim 1, wherein the compound of formula (I) is a compound of formula (Ib): 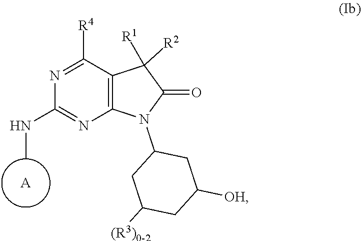
wherein R¹, R², R³, R⁴ and 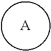
are as defined in claim 1.

9. The compound or pharmaceutically acceptable salt and/or tautomer thereof according claim 1, wherein the compound of formula (I) is a compound of formula (Ic): 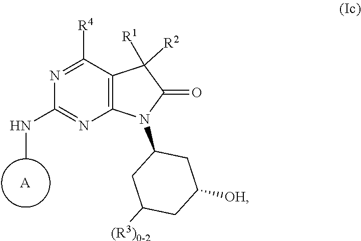
wherein R¹, R², R³, R⁴ and 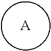
are as defined in claim 1.

10. The compound or pharmaceutically acceptable salt and/or tautomer thereof according to claim 1, wherein the compound of formula (I) is a compound of formula (Id): 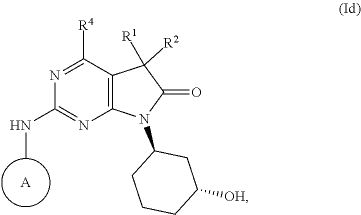
wherein R¹, R², R⁴ and 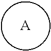
are as defined in claim 1.

11. The compound or pharmaceutically acceptable salt and/or tautomer thereof according to claim 1, wherein R¹ and R² join together to form C₃-C₄cycloalkyl or C₃-C₄cyclohaloalkyl.

12. The compound or pharmaceutically acceptable salt and/or tautomer thereof according to claim 11, wherein R¹ and R² join together to form C₃-C₄cycloalkyl.

13. The compound or pharmaceutically acceptable salt and/or tautomer thereof according to claim 12, wherein R¹ and R² join together to form C₃cycloalkyl.

14. The compound or pharmaceutically acceptable salt and/or tautomer thereof according to claim 1, wherein R⁴ is H.

15. The compound or pharmaceutically acceptable salt and/or tautomer thereof according to claim 1, wherein 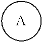
is a 5 membered heteroaryl comprising 2 heteroatoms independently selected from N, O and S, said 5 membered heteroaryl being substituted with 0 to 3 substituents R^{A}, wherein R^{A} is as defined in claim 1.

16. The compound or pharmaceutically acceptable salt and/or tautomer thereof according to claim 15, wherein 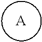
is a 5 membered heteroaryl comprising 2 heteroatoms independently selected from N and O, said 5 membered heteroaryl being substituted with 0 to 3 substituents R^{A}, wherein R^{A} is as defined in claim 1.

17. The compound or pharmaceutically acceptable salt and/or tautomer thereof according to claim 1, wherein 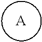
is a 5 membered heteroaryl comprising 1 to 3 heteroatoms which are each N, said 5 membered heteroaryl being substituted with 0 to 3 substituents R^{A}, wherein R^{A} is as defined in claim 1.

18. The compound or pharmaceutically acceptable salt and/or tautomer thereof according to claim 1, wherein 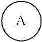
is a 5 membered heteroaryl comprising 2 heteroatoms which are each N, said 5 membered heteroaryl being substituted with 0 to 3 substituents R^{A}, wherein R^{A} is as defined in claim 1.

19. The compound or pharmaceutically acceptable salt and/or tautomer thereof according to claim 1, wherein 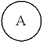
is a 5 membered heteroaryl comprising 2 heteroatoms which are each N, said 5 membered heteroaryl being substituted with 0 to 2 substituents R^{A}, wherein R^{A} is as defined in claim 1.

20. The compound or pharmaceutically acceptable salt and/or tautomer thereof according to claim 1, wherein 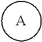
is a 5 membered heteroaryl comprising 2 heteroatoms which are each N, said 5 membered heteroaryl being substituted with 0 or 1 substituent R^{A}, wherein R^{A} is *L¹-X¹, and L¹ and X¹ are as defined in claim 1.

21. The compound or pharmaceutically acceptable salt and/or tautomer thereof according to claim 1, wherein 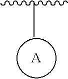
is selected from the group consisting of:

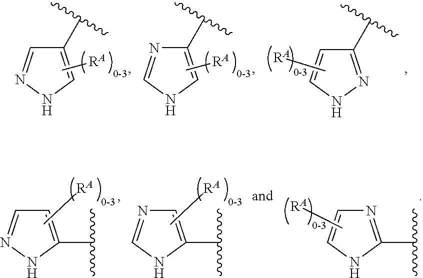

22. The compound or pharmaceutically acceptable salt and/or tautomer thereof according to claim 1, wherein 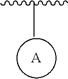
is selected from the group consisting of

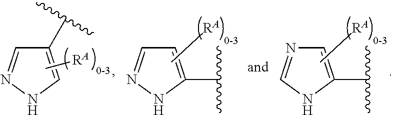

23. The compound or pharmaceutically acceptable salt and/or tautomer thereof according to claim 1, wherein 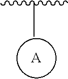
is selected from the group consisting of: 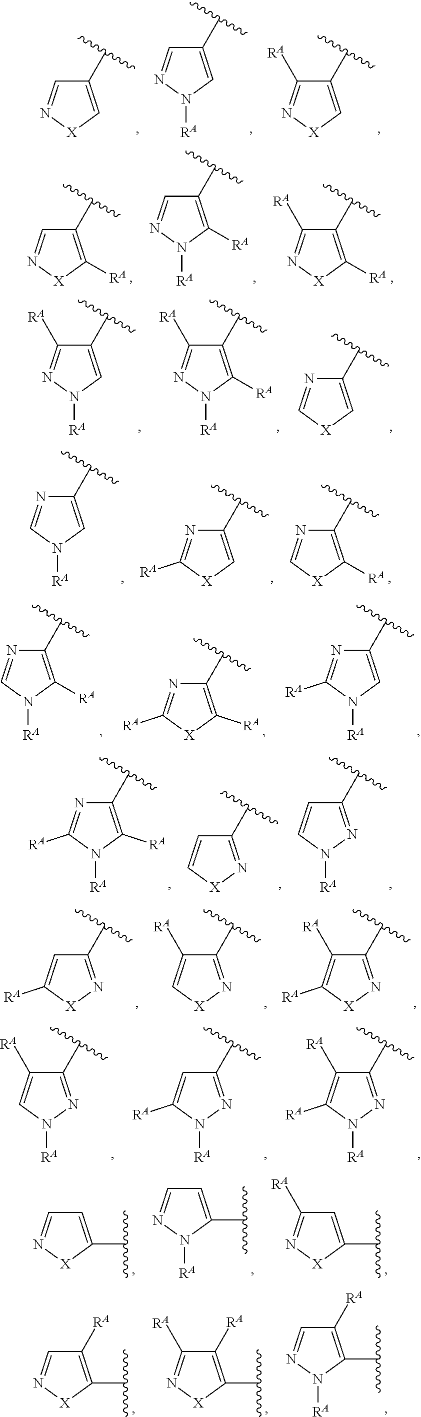 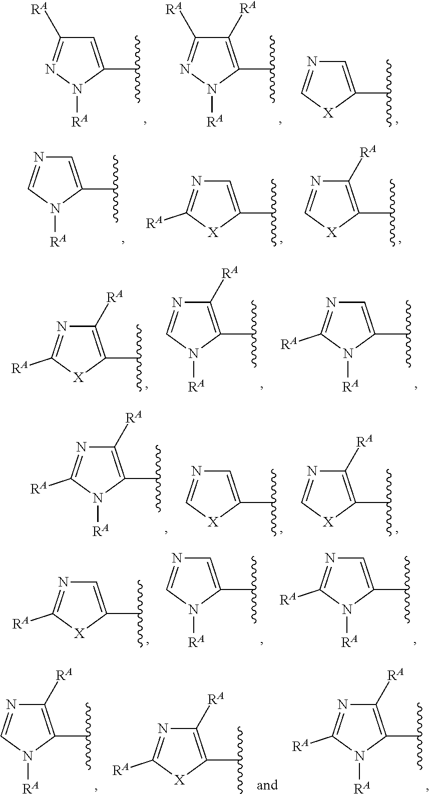
and wherein X is selected from O, NH and S.

24. The compound or pharmaceutically acceptable salt and/or tautomer thereof according to claim 1, wherein 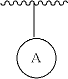
is selected from the group consisting of:

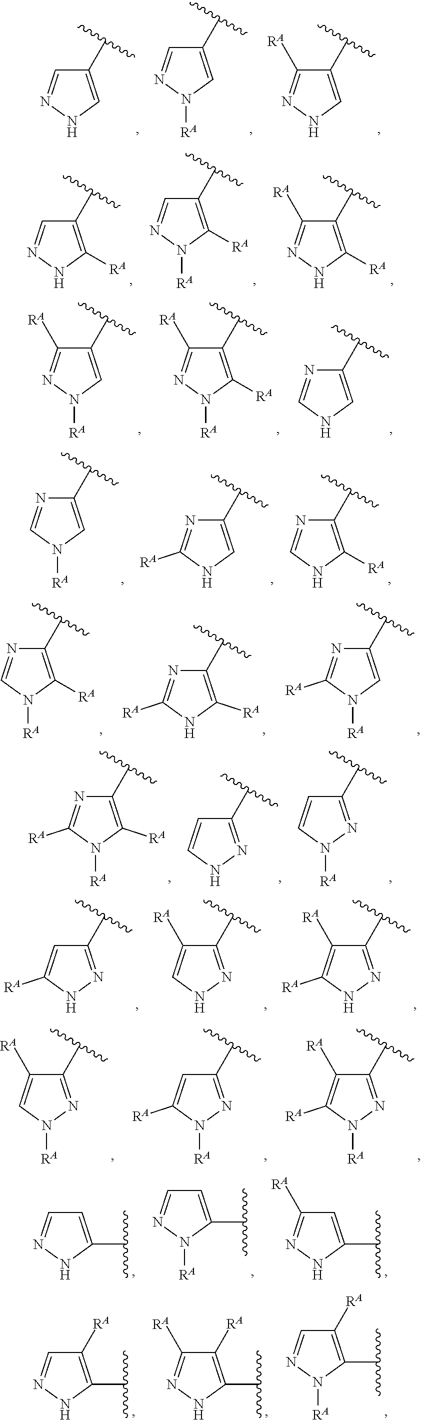

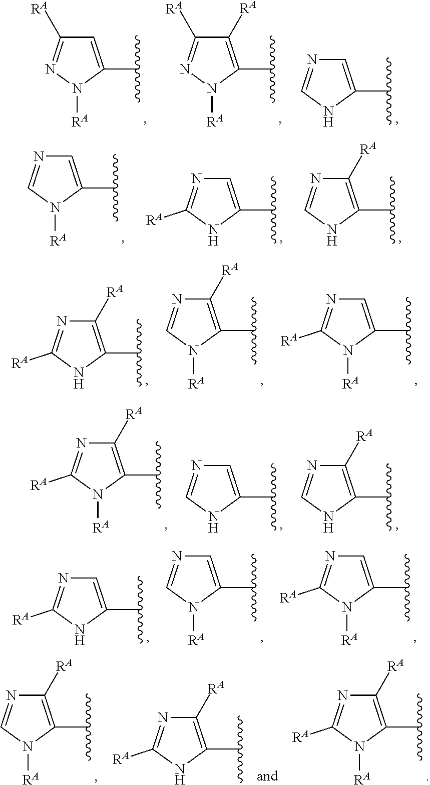

25. The compound or pharmaceutically acceptable salt and/or tautomer thereof according to claim 1, wherein 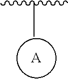
is selected from the group consisting of: 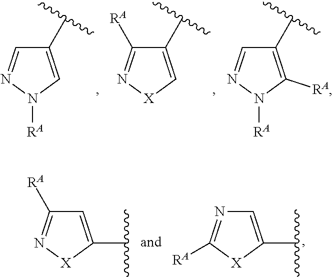
and wherein X is selected from O, NH and S.

26. The compound or pharmaceutically acceptable salt and/or tautomer thereof according to claim 1, wherein 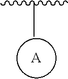
is selected from the group consisting of:

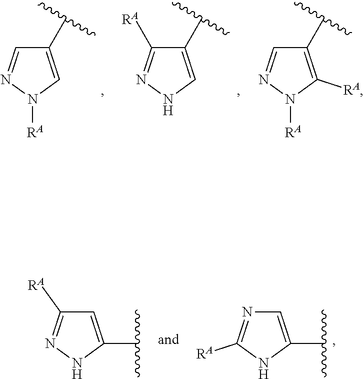

27. The compound or pharmaceutically acceptable salt and/or tautomer thereof according to claim 1, wherein:
i) 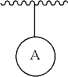
is selected from the group consisting of: 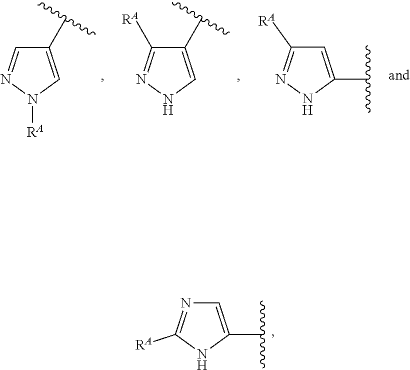
and R^{A} is selected from
a) halo,
b) cyano,
c) C₁-C₆alkyl,
d) C₁-C₆haloalkyl,
e) C₁-C₆hydroxyalkyl,
f) O—C₁-C₆alkyl,
g) C(═O)—O—C₁-C₆alkyl,
h) C₁-C₆alkylene-O—C₁-C₆alkyl,
i) O—C₁-C₆alkylene-O—C₁-C₆alkyl,
j) C₃-C₆cycloalkyl substituted by 0-3 R⁸ groups,
k) 5-10 membered heteroaryl comprising 1-4 heteroatoms independently selected from N, O and S substituted by 0-3 R⁸ groups,
l) 5-10 membered partially saturated heterocyclyl comprising 1-4 heteroatoms independently selected from N, O and S substituted by 0-3 R⁸ groups, m) O—C₃-C₆cycloalkyl substituted by 0-3 R⁸ groups,
n) C≡C-5-10 membered heteroaryl comprising 1-4 heteroatoms independently selected from N, O and S substituted by 0-3 R⁸ groups,
o) 5-5-10 membered heteroaryl comprising 1-4 heteroatoms independently selected from N, O and S substituted by 0-3 R⁸ groups,
p) C₁-C₆alkylene-5-10 membered heteroaryl comprising 1-4 heteroatoms independently selected from N, O and S substituted by 0-3 R⁸ groups,
q) 0-C₁-C₆haloalkyl,
r) O—C₁-C₆alkylene-N(C₁-C₆alkyl)₂,
s) 0-C₁-C₆hydroxyalkylene-O—C₁-C₆alkyl,
t) 0-C₁-C₆alkylene-C₃-C₆cycloalkyl substituted by 0-3 R⁸ groups,
u) O—C₁-C₆alkylene-3-6 membered heterocyclyl comprising 1 or 2 heteroatoms independently selected from O, N and S substituted by 0-3 R⁸ groups,
v) O—C₁-C₆alkylene-C(═O)—N(C₁-C₆alkyl)₂,
w) C₁-C₆alkylene-5-10 membered partially saturated heterocyclyl comprising 1-4 heteroatoms independently selected from N, O and S substituted by 0-3 R⁸ groups,
x) O—C₁-C₆alkylene-7-10 membered spiroheterocyclyl comprising 1 or 2 heteroatoms independently selected from O, N and S substituted by 0-3 R⁸ groups,
y) O—C₁-C₆alkylene-S(O)₂—C₁-C₆alkyl,
z) O—C₁-C₆hydroxyalkyl,
aa) O—C₁-C₆alkylene-5-10 membered heteroaryl comprising 1-4 heteroatoms independently selected from N, O and S substituted by 0-3 R⁸ groups,
bb) O-5-10 membered heteroaryl comprising 1-4 heteroatoms independently selected from N, O and S substituted by 0-3 R⁸ groups,
cc) O-3-6 membered heterocyclyl comprising 1 or 2 heteroatoms independently selected from O, N and S substituted by 0-3 R⁸ groups,
dd) C≡C—C₃-C₆cycloalkyl substituted by 0-3 R⁸ groups,
ee) S—C₁-C₆haloalkyl,
ff) O—C₁-C₆alkylene-O-3-6 membered heterocyclyl comprising 1 or 2 heteroatoms independently selected from O, N and S substituted by 0-3 R⁸ groups
gg) and 3 to 6 membered heterocyclyl comprising 1 or 2 heteroatoms selected from N, O and S substituted by 0-3 R⁸ groups; or
ii) 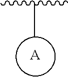
is 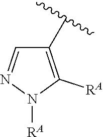
and the RA substituents join together to form, with the ring atoms to which they are attached, a 4 to 6 membered heterocyclyl comprising 1 to 3 heteroatoms independently selected from N, O and S.

28. The compound or pharmaceutically acceptable salt and/or tautomer thereof according to claim 27, wherein each R⁸ is independently selected from the group consisting of halo, C₁-C₆alkyl, hydroxyl, cyano, S(O₂)—C₁-C₆alkyl, C(═O)—C₁-C₆alkyl, O—C₁-C₆alkyl and C₁-C₆haloalkyl, or wherein two R⁸ substituents on the same ring atom join together to form ═O.

29. The compound or pharmaceutically acceptable salt and/or tautomer thereof according to claim 27, wherein:
i) 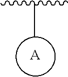
is selected from the group consisting of: 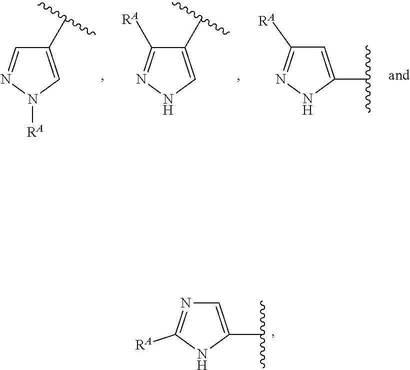
and R^{A} is selected from halo, cyano, C₁-C₆alkyl, C₁-C₆haloalkyl, C₁-C₆hydroxyalkyl, O—C₁-C₆alkyl, C(═O)—O—C₁-C₆alkyl, C₁-C₆alkylene-O—C₁-C₆alkyl, O—C₁-C₆alkylene-O—C₁-C₆alkyl, C₃-C₆cycloalkyl and 3 to 6 membered heterocyclyl comprising 1 or 2 heteroatoms selected from N, O and S; or
ii) 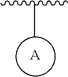
is 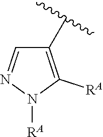
and the R^{A} substituents join together to form, with the ring atoms to which they are attached, a 4 to 6 membered heterocyclyl comprising 1 to 3 heteroatoms independently selected from N, O and S.

30. The compound or pharmaceutically acceptable salt and/or tautomer thereof according to claim 1, wherein 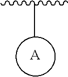
is 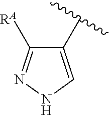
and R^{A} is CH₃, OCH₃ or OCH₂CH₃.

31. The compound or pharmaceutically acceptable salt and/or tautomer thereof according to claim 1, wherein each L¹ is independently selected from bond, O, C(═O), *C(═O)—O**, C₁-C₆alkylene, C₁-C₆haloalkylene, *O—C₁-C₆alkylene**, C₁-C₆alkylene-O—C₁-C₆alkylene, C₁-C₆hydroxyalkylene, C₃-C₆cycloalkylene, 3-6 membered heterocyclylene and O—C₁-C₆alkylene-O, wherein * indicates the point of attachment to 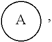
and ** indicates the point of attachment to X¹;
and each X¹ is independently selected from H, halo, cyano, hydroxyl, C₁-C₆alkyl, C₁-C₆haloalkyl, C₃-C₆cycloalkyl, O—C₁-C₆alkyl, C₁-C₆hydroxyalkyl and 3-6 membered heterocyclyl.

32. The compound or pharmaceutically acceptable salt and/or tautomer thereof according to claim 1, wherein R^{A} is selected from the list consisting of C₁-C₆alkyl, C₁-C₆alkylene-O—C₁-C₆alkyl, C₁-C₆haloalkyl, C(═O)—O—C₁-C₆alkyl, C₁-C₆hydroxyalkyl, 3-6 membered heteroatom comprising 1 heteroatom that is O, halo, 0-C₁-C₆alkyl, C₃-C₆cycloalkyl, cyano and O—C₁-C₆alkylene-O—C₁-C₆alkyl.

33. A compound selected from any one of
7′-((1R,3R)-3-hydroxycyclohexyl)-2′-((1-methyl-1H-pyrazol-4-yl)amino)spiro[cyclopropane-1,5′-pyrrolo[2,3-d]pyrimidin]-6′(7′H)-one;
7′-((1R,3R)-3-hydroxycyclohexyl)-2′-((3-methyl-1H-pyrazol-4-yl)amino)spiro[cyclopropane-1,5′-pyrrolo[2,3-d]pyrimidin]-6′(7′H)-one;
2′-((3-(difluoromethyl)-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
methyl 4-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)-1H-pyrazole-3-carboxylate;
7′-((1R,3R)-3-hydroxycyclohexyl)-2′-((3-methyl-1H-pyrazol-5-yl)amino)spiro[cyclopropane-1,5′-pyrrolo[2,3-d]pyrimidin]-6′(7′H)-one;
7′-((1R,3R)-3-hydroxycyclohexyl)-2′-((3-(hydroxymethyl)-1H-pyrazol-4-yl)amino)spiro[cyclopropane-1,5′-pyrrolo[2,3-d]pyrimidin]-6′(7′H)-one;
7′-((1R,5R)-5-hydroxy-3,3-dimethylcyclohexyl)-2′-((3-methyl-1H-pyrazol-4-yl)amino)spiro[cyclopropane-1,5′-pyrrolo[2,3-d]pyrimidin]-6′(7′H)-one;
7′-((1R,3R)-3-hydroxycyclohexyl)-2′-((3-(tetrahydrofuran-3-yl)-1H-pyrazol-4-yl)amino)spiro[cyclopropane-1,5′-pyrrolo[2,3-d]pyrimidin]-6′(7′H)-one;
7′-((1R,3R)-3-hydroxycyclohexyl)-2′-((4,5,6,7-tetrahydropyrazolo[1,5-a]pyrazin-3-yl)amino)spiro[cyclopropane-1,5′-pyrrolo[2,3-d]pyrimidin]-6′(7′H)-one;
7′-((1R,3R)-3-hydroxycyclohexyl)-2′-((3-(tetrahydro-2H-pyran-2-yl)-1H-pyrazol-4-yl)amino)spiro[cyclopropane-1,5′-pyrrolo[2,3-d]pyrimidin]-6′(7′H)-one;
7′-((1R,3R)-3-hydroxycyclohexyl)-2′-((2-(methoxymethyl)-1H-imidazol-5-yl)amino)spiro[cyclopropane-1,5′-pyrrolo[2,3-d]pyrimidin]-6′(7′H)-one;
7′-((1R,3R)-3-hydroxycyclohexyl)-2′-((3-methoxy-1H-pyrazol-4-yl)amino)spiro[cyclopropane-1,5′-pyrrolo[2,3-d]pyrimidin]-6′(7′H)-one;
2′-((3-chloro-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
2′-((3-cyclopropyl-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
7′-((1R,3R)-3-hydroxycyclohexyl)-2′-((3-(trifluoromethyl)-1H-pyrazol-4-yl)amino)spiro[cyclopropane-1,5′-pyrrolo[2,3-d]pyrimidin]-6′(7′H)-one;
7′-((1R,3R)-3-hydroxycyclohexyl)-2′-((1-(2-methoxyethyl)-1H-pyrazol-4-yl)amino)spiro[cyclopropane-1,5′-pyrrolo[2,3-d]pyrimidin]-6′(7′H)-one;
7′-(3-hydroxycycloheptyl)-2′-((3-methyl-1H-pyrazol-4-yl)amino)spiro[cyclopropane-1,5′-pyrrolo[2,3-d]pyrimidin]-6′(7′H)-one;
7′-((1R,3R)-3-hydroxycycloheptyl)-2′-((3-methyl-1H-pyrazol-4-yl)amino)spiro[cyclopropane-1,5′-pyrrolo[2,3-d]pyrimidin]-6′(7′H)-one;
2′-((3-chloro-1H-pyrazol-4-yl)amino)-7′-((1R,3R)-3-hydroxycycloheptyl)spiro[cyclopropane-1,5′-pyrrolo[2,3-d]pyrimidin]-6′(7′H)-one;
4-((7′-((1R,3R)-3-hydroxycyclohexyl)-6′-oxo-6′,7′-dihydrospiro[cyclopropane-1,5′-pyrrolo[2,3-d]pyrimidin]-2′-yl)amino)-1H-pyrazole-3-carbonitrile;
7′-((1R,3R)-3-hydroxycyclohexyl)-2′-((3-(methoxymethyl)-1H-pyrazol-4-yl)amino)spiro[cyclopropane-1,5′-pyrrolo[2,3-d]pyrimidin]-6′(7′H)-one;
7′-((1R,3R)-3-hydroxycyclohexyl)-2′-((3-(2-methoxyethoxy)-1H-pyrazol-4-yl)amino)spiro[cyclopropane-1,5′-pyrrolo[2,3-d]pyrimidin]-6′(7′H)-one;
2′-((3-ethoxy-1H-pyrazol-4-yl)amino)-7′-((1R,3R)-3-hydroxycyclohexyl)spiro[cyclopropane-1,5′-pyrrolo[2,3-d]pyrimidin]-6′(7′H)-one;
or a pharmaceutically acceptable salt and/or tautomer thereof.

34. The compound according to claim 1, wherein the compound is 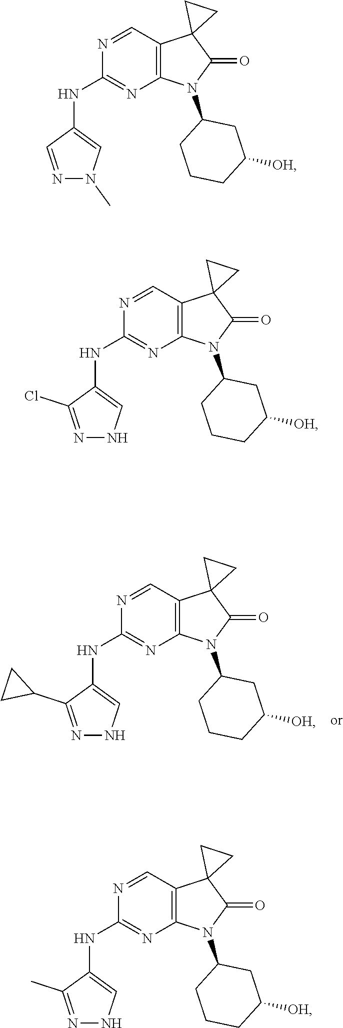
or a pharmaceutically acceptable salt and/or tautomer thereof.

35. The compound according to claim 1, wherein the compound is 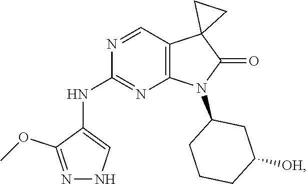 [SMILES: COc1n[nH]cc1Nc1ncc2c(n1)N([C@@H]1CCC[C@@H](O)C1)C(=O)C21CC1]
or a pharmaceutically acceptable salt and/or tautomer thereof.

36-38. (canceled)

39. The compound according to claim 1, wherein the compound is 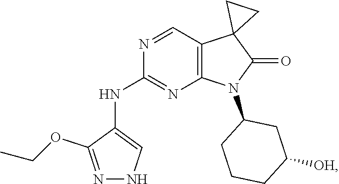 [SMILES: CCOc1n[nH]cc1Nc1ncc2c(n1)N([C@@H]1CCC[C@@H](O)C1)C(=O)C21CC1]
or a pharmaceutically acceptable salt and/or tautomer thereof.

40. A pharmaceutical composition comprising the compound or pharmaceutically acceptable salt and/or tautomer thereof according to claim 1 and one or more pharmaceutically acceptable carriers.

41. (canceled)

42. (canceled)

43. A method of treating cancer comprising administering to a subject in need thereof a therapeutically effective amount of the compound or pharmaceutically acceptable salt and/or tautomer thereof according to claim 1.

44-47. (canceled)

48. The method according to claim 43, wherein the cancer is selected from ovarian cancer, gastric cancer, uterine cancer, breast cancer, lung cancer and endometrial cancer.

49. The method according to claim 43, wherein the cancer is a cyclin E amplified cancer.
